(19) United States
(12) Patent Application Publication
(10) Pub. No.: US 2025/0282749 A1
(43) Pub. Date: Sep. 11, 2025

(54) COMPOUNDS AND METHODS OF USE

(71) Applicant: Tango Therapeutics, Inc., Boston, MA (US)

(72) Inventors: Kevin M. COTTRELL, Boston, MA (US); John P. MAXWELL, Boston, MA (US)

(21) Appl. No.: 18/833,468

(22) PCT Filed: Jan. 26, 2023
(86) PCT No.: PCT/US2023/011661

§ 371 Date: Jul. 26, 2024

Related U.S. Application Data
(60) Provisional application No. 63/303,451, filed on Jan. 26, 2022.

(51) Int. Cl. C07D 401/12; A61K 31/496; A61K 31/4985; A61K 31/5377; C07D 401/14; C07D 405/14; C07D 409/14; C07D 417/14; C07D 471/04; C07D 487/04

(52) U.S. Cl. CPC C07D 401/12; A61K 31/496; A61K 31/4985; A61K 31/5377; C07D 401/14; C07D 405/14; C07D 409/14; C07D 417/14; C07D 471/04; C07D 487/04

(57) ABSTRACT

Provided are compounds of Formula (I): (I), and pharmaceutically acceptable salts thereof, and pharmaceutical compositions, processes of preparing and methods of treating thereof; wherein Ring A, Ring B, X, R¹, R² and n are as defined herein.

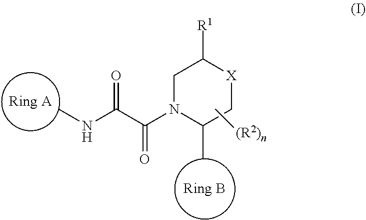

### CROSS-REFERENCE TO RELATED APPLICATION

This application claims priority to U.S. Provisional Application No. 63/303,451, filed on Jan. 26, 2022, which is incorporated by reference herein in its entirety and for all purposes.

### FIELD

Provided herein are compounds, and compositions and methods thereof. In some embodiments, provided are compounds for inhibiting protein arginine methyltransferase 5 (PRMT5). In some embodiments, provided are methods for treatment of diseases or disorders, such as cancer.

### BACKGROUND

Protein arginine methyltransferase 5 (PRMT5) is a type II arginine methyltransferase that regulates essential cellular functions, including the regulation of cell cycle progression, apoptosis and the DNA-damage response (Koh, C. et al., Curr Mol Bio Rep 2015; Wu et al., Nat Rev Drug Discovery 2021). MTAP is a critical enzyme in the methionine salvage pathway, a six-step process that recycles methionine from the product of polyamine synthesis, methylthioadenosine (MTA). Loss of MTAP causes the accumulation of its substrate, MTA, which has been described to function as a SAM-competitive PRMT5 inhibitor (Kruykov et al., 2016; Marjon et al., 2016 and Markarov et al., 2016). Data from genome-wide genetic perturbation screens using shRNA suggests a selective requirement for PRMT5 activity particularly inMTAP-deleted cancer cell lines (Kruykov et al., 2016; Marjon et al., 2016 and Markarov et al., 2016). It is proposed that the accumulation of MTA caused by MTAP-deletion in these cell lines partially inhibits PRMT5, rendering those cells selectively sensitive to additional PRMT5 inhibition.

A PRMT5 inhibitor that leverages the accumulation of MTA by binding in an MTA-uncompetitive, non-competitive or mixed mode manner or in a MTA-cooperative binding manner may demonstrate selectivity for MTAP-deleted tumor cells. Some PRMT5 inhibitors are currently being explored for therapeutic uses (e.g., for treating cancer), however there are currently no such PRMT5 therapies approved by the United States Food and Drug Administration that demonstrate selectivity for MTAP-deleted cancer cell lines.

Accordingly, there is a need for PRMT5 inhibitors for treating diseases, such as cancers.

### SUMMARY

In one embodiment, provided herein are compounds of Formula (I):

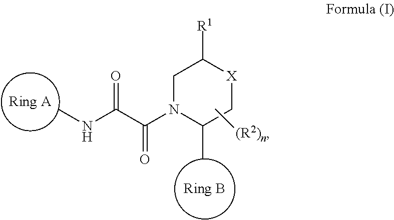

or pharmaceutically acceptable salts thereof, wherein;
X is selected from the group consisting of the group consisting of —O— and —NR⁷—;
Ring A is selected from the group consisting of an optionally substituted fused bicyclic 8-10 membered heteroaryl ring system containing at least one nitrogen atom, wherein the 8-10 membered refers to the total number of atoms in the fused system and optionally substituted pyridin-3-yl;
Ring B is selected from the group consisting of C₆-C₁₀ aryl and 5-10 membered heteroaryl, each optionally substituted at any available position;
each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1} and —S(═O)₂N(Ra);
each R² is independently selected from the group consisting of H, -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring);
each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂, wherein each alkyl, cycloalkyl, heterocyclyl, aryl, heteroaryl, arylalkyl and heteroarylalkyl is optionally substituted at any available position;
each R^{a1}, R^{a2} and R^{a7} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR^{b}, —NRbC(═O)R^{cc}, —NRbC(═O)OR^{b}, —C(═O)N(R^{b})₂, —OC(═O)N(R^{b})₂, —S(═O)R^{cc}, —S(═O)₂R^{b}, —SRb, —S(═O)(═NR)R, —NRbS(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl); and
n is 0, 1, 2 or 3.

In one embodiment, provided is a pharmaceutical composition comprising a compound of Formula (I), or a pharmaceutically acceptable salt thereof, as defined in any of the embodiments described herein and a pharmaceutically acceptable carrier. In some embodiments, the pharmaceutical composition further comprises a second therapeutic agent.

In one embodiment, provided is a method of treating an MTAP-deficient and/or an MTA-accumulating disease in a subject in need thereof by administering to the subject an effective amount (e.g., a therapeutically effective amount) of compound of Formula (I), or a pharmaceutically acceptable salt thereof, as defined in any of the embodiments described herein or a pharmaceutically acceptable composition thereof. In some embodiments, the compound or composition is administered in combination with a second therapeutic agent.

In one embodiment, provided is a method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering an effective amount (e.g., a therapeutically effective amount) of a compound of Formula (I) as defined in any of the embodiments described herein or a pharmaceutical composition thereof to the subject identified in step b).

In an embodiment, provided is a use of a compound of Formula (I), or a pharmaceutically acceptable salt thereof, as defined in any of the embodiments described herein, or of a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a compound of Formula (I), or a pharmaceutically acceptable salt thereof, as defined in any of the embodiments described herein, or a pharmaceutically acceptable composition as described herein for treating an MTAP-deficient and/or an MTA-accumulating disease in a subject in need thereof. In an embodiment, the compound or composition is configured to be administered in combination with a second therapeutic agent.

In an embodiment, provided is a use of a compound of compound of Formula (I), or a pharmaceutically acceptable salt thereof, as defined in any of the embodiments described herein, or of a pharmaceutically acceptable composition as described herein in the manufacturing of a medicament for treating an MTAP-deficient and/or an MTA-accumulating disease in a subject in need thereof. In an embodiment, the medicament is configured to be administered in combination with a second therapeutic agent.

### DETAILED DESCRIPTION

The disclosure herein sets forth exemplary methods, parameters and the like. It should be recognized, however, that such description is not intended as a limitation on the scope of the present disclosure but is instead provided as a description of exemplary embodiments.

As generally described herein, provided are compounds (e.g., compounds of Formula (I) or compounds of Table 1, or pharmaceutically acceptable salts thereof) that are MTA-uncompetitive PRMT5 inhibitors useful for treating proliferating disorders (e.g., cancers) associated with MTAP deficiencies and/or MTA accumulation.

In some embodiments, provided are compounds (e.g., compounds of Formula (I) or compounds of Table 1, or pharmaceutically acceptable salts thereof) that are MTA-uncompetitive, non-competitive or mixed mode PRMT5 inhibitor or an MTA cooperative binding agent useful for treating proliferating disorders (e.g., cancers) associated with MTAP deficiencies and/or MTA accumulation.

### Definitions

As used in the present disclosure, the following words and phrases are generally intended to have the meanings as set forth below unless expressly indicated otherwise or the context in which they are used indicates otherwise.

### Mtap

“MTAP” as used herein refers to methylthioadenosine phosphorylase, an enzyme in the methionine salvage pathway, also known as S-methyl-5′-thioadenosine phosphorylase; also known as BDMF; DMSFH; DMSMFH; LGMBF; MSAP; and c86fus. External IDs: OMIM: 156540 MGI: 1914152 HomoloGene:1838 chEMBL: 4941 GeneCards: MTAP Gene; Entrez 4507; RefSeq (mRNA): NM—002451; location: Chr 9: 21.8-21.93 Mb. By “wild-type” MTAP is meant that encoded by NM—002451 or having the same amino acid sequence (NP—002442). (Schmid et al. Oncogene 2000, 19, pp 5747-54).

As used herein, the term “MTAP-deficient”, “MTAP-deficiency”, “MTAP-null” and the like refer to cells (including, but not limited to, cancer cells, cell lines, tissues, tissue types, tumors, etc.) that have a significant reduction in post-translational modification, production, expression, level, stability and/or activity of MTAP relative to that in a control, e.g., reference or normal or non-cancerous cells. The reduction can be at least about 20%, 30%, 40%, 50%, 60%, 70%, 80% or 90%. In some embodiments, the reduction is at least 20%. In some embodiments, the reduction is at least 50%. The terms “MTAP-deficient and/or MTA accumulating”, “MTAP-deficient and/or MTA-accumulating”, MTAP deficient and/or MTA upregulated” and the like, regarding a cell or cells, etc., indicate that the cell or cells, etc., either are deficient in MTAP and/or overproduce or accumulate MTA. MTAP-deficient cells include those wherein the MTAP gene has been mutated, deleted, or transcriptionally silenced. As a non-limiting example, MTAP-deficient cells can have a homozygous deletion. MTAP knockdown is not lethal. In some embodiments, the MTAP-deficient cells are also CDKN2A-deficient. The MTAP deficiency can be detected using any reagent or technique known in the art, for example: immunohistochemistry utilizing an antibody to MTAP, and/or genomic sequencing, and/or nucleic acid hybridization and/or amplification utilizing at least one probe or primer comprising a sequence of at least 12 contiguous nucleotides (nt) of the sequence of MTAP, wherein the primer is no longer than about 30 nt.

An “MTAP-deficiency-related” or “MTAP-deficiency” or “MTAP deficient” disease (for example, a proliferating disease, e.g., a cancer) or a disease (for example, a proliferating disease, e.g., a cancer) “associated with MTAP deficiency” or a disease (for example, a proliferating disease, e.g., a cancer) “characterized by MTAP deficiency” and the like refer to an ailment (for example, a proliferating disease, e.g., a cancer) wherein a significant number of cells are MTAP-deficient. For example, in a MTAP-deficiency-related disease, one or more disease cells can have a significantly reduced post-translational modification, production, expression, level, stability and/or activity of MTAP. Examples of MTAP-deficiency-related diseases include, but are not limited to, cancers, including but not limited to: glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not. Similarly, some disease cells may be MTA-accumulating while others are not. Thus, the present disclosure encompasses methods of treatment involving diseases of these tissues, or any other tissues, wherein the proliferation of MTAP-deficient and/or MTA-accumulating cells can be inhibited by administration of a PRMT5 inhibitor. Some cancer cells which are MTAP-deficient are also deficient in CDKN2 Å; the post-translational modification, production, expression, level, stability and/or activity of the CDKN2A gene or its product are decreased in these cells. The genes for MTAP and CDKN2A are in close proximity on chromosome 9p21; MTAP is located approximately 100 kb telomeric to CDKN2A. Many cancer cell types harbor CDKN2A/MTAP loss (loss of both genes). Thus, in some embodiments, a MTAP-deficient cell is also deficient in CDKN2A.

### MTA and MTA Accumulation

By “MTA” is meant the PRMT5 inhibitor also known as methyl-thioadenosine, S-methyl-5′-thioadenosine, [5′deoxy-5′-(methylthio)-fl-D-ribofuranosyl]adenine, 5′-methyl-thioadenosine, 5′-deoxy, 5′-methyl thioadenosine, and the like. MTA selectively inhibits PRMT5 methyltransferase activity. MTA is the sole known catabolic substrate for MTAP. The terms “MTA accumulating”, “MTA overproducing”, “MTA upregulated” and the like refer to cells (including, but not limited to, cancer cells, cell lines, tissues, tissue types, tumors, etc.) that have a significantly increased production, level and/or stability of MTA. MTA-accumulating cells include those wherein the cells comprise at least about 20%, 30%, 40%, 50%, 60%, 70%, 80%, 90%, 100%, or greater than 100%, higher production, level and/or stability of MTA than that in normal or non-cancerous cells. In some embodiments, MTA-accumulating cells include those wherein the cells comprise at least 20% higher production, level and/or stability of MTA than that in normal or non-cancerous cells. In some embodiments, MTA-accumulating cells include those wherein the cells comprise at least 50% higher production, level and/or stability of MTA than that in normal or non-cancerous cells. Determination of MTA accumulation in test samples (e.g., cells such as cancer cells being tested for MTA accumulation) and reference samples, and other cells, tissues, samples, etc., can be performed using any method known in the art. Such methods for detecting MTA include, as a non-limiting example, liquid chromatography-electrospray ionization-tandem mass spectrometry (LC-ESI-MS/MS), as described in Stevens et al. J. Chromatogr. A. 2010, 1217, pp 3282-3288; and Kirovski et al. Am. J. Pathol. 2011, 178, pp 1145-1152; and references cited therein. Loss of MTAP is associated with accumulation of MTA (Williams-Ashman et al. Biochem. Pharm. 1982, 31, pp 277-288; and Limm et al. Eur. J Cancer. 2013, 49, Issue 6.

An “MTA-accumulation-related”, “MTA-accumulation”, “MTA-accumulating”, “MTA overproducing”, “MTA upregulated” disease (for example, a proliferating disease, e.g., a cancer) or a disease (for example, a proliferating disease, e.g., a cancer) “associated with MTA accumulation” or a disease (for example, a proliferating disease, e.g., a cancer) “characterized by MTA accumulation” and the like refer to an ailment (for example, a proliferating disease, e.g., a cancer) wherein a significant number of cells are MTA accumulating. Examples of MTA-accumulating diseases include, but are not limited to, cancers, including but not limited to: glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma (See FIG. 1). In a patient afflicted with a MTAP-deficiency-related disease, it is possible that some disease cells (e.g., cancer cells) can be MTAP-deficient while others are not.

In a patient having or having been diagnosed with an MTA-accumulating disease, some cells may be MTA-accumulating while others are not.

An increase in therapeutic window between normal cells and MTAP-deleted/MTA accumulating cells could be achieved by using an inhibitor that binds PRMT5 uncompetitively with MTA. As used herein, “uncompetitive binding” and “uncompetitive inhibition” and “cooperative binding” and “cooperative inhibition” (e.g., MTA-uncompetitive binding, MTA-uncompetitive inhibition, MTA-cooperative binding, MTA-cooperative inhibition) refers to binding of an inhibitor to a protein (e.g., PRMT5) that is increased in the presence of a co-factor (e.g., MTA) over the binding of the same inhibitor in the absence of the co-factor. The PRMT5 inhibitors known in the art are generally either SAM (S-adenosylmethionine) uncompetitive or SAM competitive. As the concentration of SAM in wild-type and MTAP-null cells is similar, these inhibitors are expected to bind with similar potency to both cell types. By contrast, an MTA-cooperative (and either SAM competitive or showing enhanced cooperativity with MTA relative to SAM) inhibitor would bind with apparent greater potency in the presence of high concentrations of MTA and would therefore result in preferential inhibition of PRMT5 in MTA-accumulating cells relative to normal cells.

As described further herein, a cancer cell, a cancer type, or a subject with cancer, is “PRMT5 inhibitor sensitive,” sensitive to treatment with PRMT5 inhibitors,” sensitive to PRMT5 therapeutic inhibition,” or described in similar terms if it is amenable to treatment with a PRMT5 inhibitor, e.g., due to its MTAP deficiency and/or MTA accumulation character.

### PRMT5

“PRMT5” as used herein is the gene or protein Protein Arginine Methyltransferase 5, also known as HRMT1L5; IBP72; JBP1; SKB1; or SKB1Hs External IDs: OMIM: 604045, MGI: 1351645, HomoloGene: 4454, ChEMBL: 1795116, GeneCards: PRMT5 Gene; EC number 2.1.1.125. Ensembl ENSG00000100462; UniProt 014744; Entrez Gene ID: 10419; RefSeq (mRNA): NM—001039619. The mouse homolog is NM—013768.

Methyltransferases such as PRMT5 catalyze the transfer of one to three methyl groups from the co-factor S-adenosylmethionine (also known as SAM or AdoMet) to lysine or arginine residues of histone proteins. Arginine methylation is carried out by 9 different protein arginine methyltransferases (PRMT) in humans. Three types of methylarginine species exist: (1) Monomethylarginine (MMA); (2) Asymmetric dimethyl arginine (ADMA), which is produced by Type I methyl transferases (PRMT1, PRMT2, PRMT3, CARM1, PRMT6 and PRMT8); and (3) Symmetrical dimethylarginine (SDMA), which is produced by Type II methyl transferases (PRMT5 and PRMT7). PRMT1 and PRMT5 are the major asymmetric and symmetric arginine methyltransferases, respectively. PRMT5 promotes symmetric dimethylation on histones at H3R8 and H4R3 (H4R3me2). Symmetric methylation of H4R3 is associated with transcriptional repression and can act as a binding site for DNMT3A. Loss of PRMT5 results in reduced DNMT3A binding and gene activation. Tumor suppressor gene ST7 and chemokines RNATES, IP10, CXCL11 are targeted and silenced by PRMT5. WO 2011/079236.

Additional substrates include E2F1, p53, EGFR and CRAF. PRMT5 is part of a multi-protein complex comprising the co-regulatory factor WDR77 (also known as MEP50, a CDK4 substrate) during GUS transition. Phosphorylation increases PRMT5/WDR77 activity. WDR77 is the non-catalytic component of the complex and mediates interactions with binding partners and substrates. PRMT5 can also interact with pICIn or RioK1 adaptor proteins in a mutually exclusive fashion to modulate complex composition and substrate specificity.

PRMT5 has either a positive or negative effect on its substrates by arginine methylation when interacting with a number of complexes and is involved in a variety of cellular processes, including RNA processing, signal transduction, transcriptional regulation, and germ cell development. PRMT5 is a major pro-survival factor regulating eIF4E expression and p53 translation. PRMT5 triggers p53-dependent apoptosis and sensitized various cancer cells to Tumor necrosis factor (TNF)-related apoptosis-inducing ligand (TRAIL) without affecting TRAIL resistance in non-transformed cells.

The term “PRMT5 inhibitor” refers to any compound capable of inhibiting the production, level, activity, expression or presence of PRMT5. These include, as non-limiting examples, any compound inhibiting the transcription of the gene, the maturation of RNA, the translation of mRNA, the posttranslational modification of the protein, the enzymatic activity of the protein, the interaction of same with a substrate, etc. The term also refers to any agent that inhibits the cellular function of the PRMT5 protein, either by ATP-competitive inhibition of the active site, allosteric modulation of the protein structure, disruption of protein-protein interactions, or by inhibiting the transcription, translation, post-translational modification, or stability of PRMT5 protein.

In some embodiments, a PRMT5 inhibitor competes with another compound, protein or other molecule which interacts with PRMT5 and is necessary for PRMT5 function. As a non-limiting example, a PRMT5 inhibitor can compete with the co-factor S-adenosylmethionine (also known as SAM or AdoMet).

In some embodiments, the PRMT5 inhibitor is uncompetitive with MTA. In some embodiments, the PRMT5 inhibitor is uncompetitive with MTA and competitive with SAM. In some embodiments, the PRMT5 inhibitor is uncompetitive with MTA and uncompetitive with SAM but binds with a higher degree of potency for the MTA complex relative to the SAM complex.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L.

Eliel, Ed., Univ. of Notre Dame Press, Notre Dame, IN 1972). Additionally encompassed are compounds described herein as individual isomers substantially free of other isomers, and alternatively, as mixtures of various isomers.

The “enantiomeric excess” (“e.e.”) or “% enantiomeric excess” (“% e.e.”) of a composition as used herein refers to an excess of one enantiomer relative to the other enantiomer present in the composition. For example, a composition can contain 90% of one enantiomer, e.g., the S enantiomer, and 10% of the other enantiomer, i.e., the R enantiomer.

$$e . e . = ( 90 - 10 ) / 100 = 80 \% .$$

Thus, a composition containing 90% of one enantiomer and 10% of the other enantiomer is said to have an enantiomeric excess of 80%.

The “diastereomeric excess” (“d.e.”) or “% diastereomeric excess” (“% d.e.”) of a composition as used herein refers to an excess of one diastereomer relative to one or more different diastereomers present in the composition. For example, a composition can contain 90% of one diastereomer, and 10% of one or more different diastereomers.

$$d . e . = ( 90 - 10 ) / 100 = 80 \% .$$

Thus, a composition containing 90% of one diastereomers and 10% of one or more different diastereomers is said to have a diastereomeric excess of 80%.

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

In a formula, [symbol] is a single bond where the stereochemistry of the moieties immediately attached thereto is not specified.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

The term “unsaturated bond” refers to a double or triple bond.

The term “unsaturated” or “partially unsaturated” refers to a moiety that includes at least one double or triple bond.

The term “saturated” refers to a moiety that does not contain a double or triple bond, i.e., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

The term “azido” refers to the radical —N₃. “Aliphatic” refers to an alkyl, alkenyl, alkynyl, or carbocyclyl group, as defined herein. “Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group (e.g., a 3-10 membered heterocyclyl containing 1, 2 or 3 heteroatoms selected from N, O, S and oxidized forms thereof). In some embodiments, a heterocyclylalkyl is a C₁₋₂ alkyl-heterocyclyl (e.g., —CH₂-heterocyclyl, —CH₂CH₂-heterocyclyl, —CH(CH₃)-heterocyclyl). In some embodiments, a heterocyclylalkyl is a —CH₂-heterocyclyl. Typical heterocyclylalkyl groups include, but are not limited to, tetrahydrofuranylmethyl, tetrahydropyranylmethyl, pyrrolidinylmethyl, piperidinylmethyl, piperazinylmethyl, morpholinylmethyl, pyrrolidinylethyl, piperidinylethyl, piperazinylethyl, morpholinylethyl, and the like. “Aralkyl” or “arylalkyl” is a subset of alkyl and aryl, as defined herein, and refers to an alkyl group substituted by an aryl group (e.g., a C₆-C₁₀ aryl group). In some embodiments, arylalkyl is a C₁₋₂ alkyl-aryl (e.g., —CH₂-aryl, —CH₂CH₂-aryl, —CH(CH₃)-aryl). In some embodiments, arylalkyl is a —CH₂-aryl (e.g., —CH₂-phenyl, —CH₂-naphthyl). “Alkyl” refers to a radical of a straight-chain or branched saturated hydrocarbon group having from 1 to 20 carbon atoms (“C₁₋₂₀ alkyl” or “C₁-C₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”).

In some embodiments, an alkyl group has 1 to 6 carbon atoms (“C₁₋₆ alkyl”, also referred to herein as “lower alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C), and n-hexyl (C). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁₋₁₀ alkyl (e.g., —CH₃). In certain embodiments, the alkyl group is substituted C₁₋₁₀ alkyl.

Common alkyl abbreviations include Me (—CH₃), Et (—CH₂CH₃), ⁱPr (—CH(CH₃)₂), ^{t}Pr (—CH₂CH₂CH₃), ^{t}Bu (—CH₂CH₂CH₂CH₃), or ^{t}Bu (—CH₂CH(CH₃)₂).

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more alkyl (methyl) groups, include but are not limited to, substituted methylene (—CH(CH₃)—, (—C(CH₃)₂—), substituted ethylene (—CH(CH₃)CH₂—, —CH₂CH(CH₃)—, —C(CH₃)₂CH₂—, —CH₂C(CH₃)₂—), substituted propylene (—CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—), and the like. When a range or number of carbons is provided for a particular alkylene group, it is understood that the range or number refers to the range or number of carbons in the linear carbon divalent chain. Alkylene groups may be substituted or unsubstituted with one or more substituents as described herein.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₈), pentadienyl (C₈), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”).

In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆ alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroC₁₋₂ alkyl”).

In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl. Exemplary heteroalkyl groups include: —CH₂OH, —CH₂OCH₃, —CH₂NH₂, —CH₂NH(CH₃), —CH₂N(CH₃)₂, —CH₂CH₂OH, —CH₂CH₂OCH₃, —CH₂CH₂NH₂, —CH₂CH₂NH(CH₃), —CH₂CH₂N(CH₃)₂.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group is substituted with one or more of groups selected from halo, C₁-C₈ alkyl, C₁-C₈ haloalkyl, cyano, hydroxy, C₁-C₈ alkoxy, and amino.

Examples of representative substituted aryls include the following

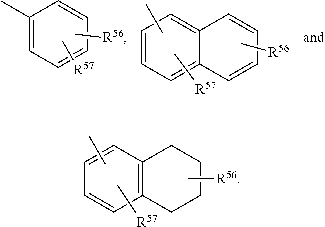

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁸SO₂R⁵⁹, COOalkyl, COOaryl, CONR⁵⁸R⁵⁹, CONR⁵⁸OR⁵⁹, NR⁸R9, SO₂NR⁵⁸R⁵⁹, S-alkyl, SOalkyl, SO₂alkyl, Saryl, SOaryl, SO₂aryl; or R⁵⁶ and R⁵⁷ may be joined to form a cyclic ring (saturated or unsaturated) from 5 to 8 atoms, optionally containing one or more heteroatoms selected from the group consisting of N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.
“Fused aryl” refers to an aryl having two of its ring carbons in common with a second aryl or heteroaryl ring or with a carbocyclyl or heterocyclyl ring.

“Heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, In such instances, unless otherwise specified, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl. In some embodiments, a heteroaryl group is a bicyclic 8-12 membered aromatic ring system having ring carbon atoms and 1-6 ring heteroatoms provided in the aromatic ring system, wherein each heteroatom is independently selected from nitrogen, oxygen, and sulfur (“8-12 membered bicyclic heteroaryl”). In some embodiments, a heteroaryl group is an 8-10 membered bicyclic aromatic ring system having ring carbon atoms and 1-6 ring heteroatoms provided in the aromatic ring system, wherein each heteroatom is independently selected from nitrogen, oxygen, and sulfur (“8-10 membered bicyclic heteroaryl”). In some embodiments, a heteroaryl group is a 9-10 membered bicyclic aromatic ring system having ring carbon atoms and 1-6 ring heteroatoms provided in the aromatic ring system, wherein each heteroatom is independently selected from nitrogen, oxygen, and sulfur (“9-10 membered bicyclic heteroaryl”). Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

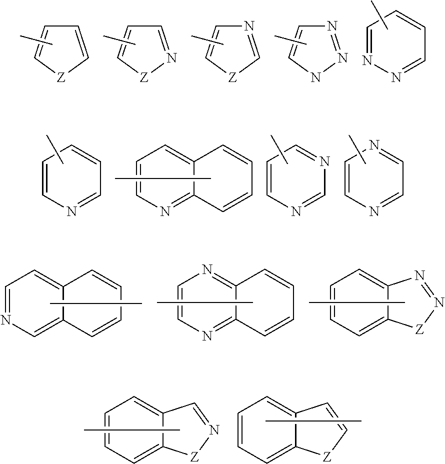

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-membered heteroaryl.

In the structures described herein, a substituent attached to a polycyclic (e.g., bicyclic or tricyclic) cycloalkyl, heterocyclyl, aryl or heteroaryl with a bond that spans two or more rings is understood to mean that the substituent can be attached at any position in each of the rings.

“Heteroaralkyl” or “heteroarylalkyl” is a subset of “alkyl” and refers to an alkyl group substituted by a heteroaryl group (e.g., a 5-10 membered heteroaryl containing 1, 2 or 3 heteroatoms selected from O, N and S and oxidized forms thereof), wherein the point of attachment is on the alkyl moiety. In some embodiments, a heteroarylalkyl is a C₁₋₂ alkyl-heteroaryl (e.g., —CH₂-heteroaryl, —CH₂CH₂-heteroaryl, —CH(CH₃)-heteroaryl). In some embodiments, a heteroarylalkyl is a —CH₂-heteroaryl. Typical heteroarylalkyl groups include, but are not limited to, pyridinylmethyl, pyrimidinylmethyl, furanylmethyl, thiophenylmethyl, pyrolylmethyl, pyrazolylmethyl, imidazolylmethyl, thiazolylmethyl, oxazolylmethyl, thiazolylmethyl, pyridinylethyl, pyrimidinylethyl, furanylethyl, thiophenylethyl, pyrolylethyl, pyrazolylethyl, imidazolylethyl, thiazolylethyl, oxazolylethyl, thiazolylethyl and the like.

The term “carbocyclyl” or “carbocyclic” refers to a radical of a non-aromatic monocyclic, bicyclic, or tricyclic or polycyclic hydrocarbon ring system having from 3 to 14 ring carbon atoms (“C₃₋₁₄ carbocyclyl”) and zero heteroatoms in the non-aromatic ring system. Carbocyclyl groups include fully saturated ring systems (e.g., cycloalkyls), and partially saturated ring systems. In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₈), cyclopentenyl (C₈), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like.

As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

The term “cycloalkyl” as employed herein includes saturated cyclic, bicyclic, tricyclic, or polycyclic hydrocarbon groups having 3 to 14 carbons containing the indicated number of rings and carbon atoms (for example a C₃-C₁₄ monocyclic, C₄-C₁₄ bicyclic, C₅-C₁₄tricyclic, or C₆-C₁₄ polycyclic cycloalkyl). In some embodiments “cycloalkyl” is a monocyclic cycloalkyl. In some embodiments, a monocyclic cycloalkyl has 3-14 ring carbon atoms. (“C₃₋₁₄ monocyclic cycloalkyl”). In some embodiments, a monocyclic cycloalkyl group has 3 to 10 ring carbon atoms (“C₃₋₁₀ monocyclic cycloalkyl”). In some embodiments, a monocyclic cycloalkyl group has 3 to 8 ring carbon atoms (“C₃₋₈ monocyclic cycloalkyl”). In some embodiments, a monocyclic cycloalkyl group has 3 to 6 ring carbon atoms (“C₃₋₆ monocyclic cycloalkyl”). In some embodiments, a monocyclic cycloalkyl group has 4 to 6 ring carbon atoms (“C₄₋₆ monocyclic cycloalkyl”). In some embodiments, a monocyclic cycloalkyl group has 5 to 6 ring carbon atoms (“C₅₋₆ monocyclic cycloalkyl”). In some embodiments, a monocyclic cycloalkyl group has 5 to 10 ring carbon atoms (“C₅₋₁₀ monocyclic cycloalkyl”). Examples of monocyclic C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈).

In some embodiments “cycloalkyl” is a bicyclic cycloalkyl. In some embodiments, a bicyclic cycloalkyl has 4-14 ring carbon atoms. (“C₄₋₁₄ bicyclic cycloalkyl”). In some embodiments, a bicyclic cycloalkyl group has 4 to 12 ring carbon atoms (“C₄₋₁₂ bicyclic cycloalkyl”). In some embodiments, a bicyclic cycloalkyl group has 4 to 10 ring carbon atoms (“C₄₋₁₀ bicyclic cycloalkyl”). In some embodiments, a bicyclic cycloalkyl group has 5 to 10 ring carbon atoms (“C₅₋₁₀ bicyclic cycloalkyl”). In some embodiments, a bicyclic cycloalkyl group has 6 to 10 ring carbon atoms (“C₆₋₁₀ bicyclic cycloalkyl”). In some embodiments, a bicyclic cycloalkyl group has 8 to 10 ring carbon atoms (“C₅₋₁₀ bicyclic cycloalkyl”). In some embodiments, a bicyclic cycloalkyl group has 7 to 9 ring carbon atoms (“C₇₋₉ bicyclic cycloalkyl”). Examples of bicyclic cycloalkyls include bicyclo[1.1.0]butane (C₄), bicyclo[1.1.1]pentane (C₈), spiro[2.2]pentane (C₈), bicyclo[2.1.0]pentane (C₈), bicyclo[2.1.1]hexane (C₆), bicyclo[3.1.0]hexane (C₆), spiro[2.3]hexane (C₆), bicyclo[2.2.1]heptane (norbornane) (C₇), bicyclo[3.2.0]heptane (C₇), bicyclo[3.1.1]heptane (C₇), bicyclo[3.1.1]heptane (C₇), bicyclo[4.1.0]heptane (C₇), spiro[2.4]heptane (C₇), Spiro [3.3]heptane (C₇), bicyclo[2.2.2]octane (C₈), bicyclo[4.1.1]octane (C₈)octahydropentalene (C₈), bicyclo[3.2.1]octane (C₈), bicyclo[4.2.0]octane (C₈), spiro[2.5]octane (C₈), Spiro[3.4]octane (C₈), bicyclo[3.3.1]nonane (C₉), octahydro-1H-indene (C₉), bicyclo[4.2.1]nonane (C₉), spiro[3.5]nonane (C₉), spiro[4.4]nonane (C₉), bicyclo[3.3.2]decane (C₁₀), bicyclo[4.3.1]decane (C₁₀), spiro[4.5]decane (C₁₀), bicyclo[3.3.3]undecane (C₁₁), decahydronaphthalene (C₁₀), bicyclo[4.3.2]undecane (C₁₁), spiro[5.5]undecane (C₁₁) and bicyclo[4.3.3]dodecane (C₁₂).

In some embodiments “cycloalkyl” is a tricyclic cycloalkyl. In some embodiments, a tricyclic cycloalkyl has 6-14 ring carbon atoms. (“C₆₋₁₄ tricyclic cycloalkyl”). In some embodiments, a tricyclic cycloalkyl group has 8 to 12 ring carbon atoms (“C₈₋₁₂ tricyclic cycloalkyl”). In some embodiments, a tricyclic cycloalkyl group has 10 to 12 ring carbon atoms (“C₁₀₋₁₂ tricyclic cycloalkyl. Examples of tricyclic cycloalkyls include adamantine (C₁₂).

Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In some embodiments, the heterocyclyl is a 3- to 10-membered non-aromatic ring system having ring carbon atoms and 1, 2 or 3 heteroatoms independently selected from nitrogen, oxygen and sulfur, including oxidized forms thereof. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, aziridinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. “Nitrogen-containing heterocyclyl” group means a 4- to 7-membered non-aromatic cyclic group containing at least one nitrogen atom, for example, but without limitation, morpholine, piperidine (e.g., 2-piperidinyl, 3-piperidinyl and 4-piperidinyl), pyrrolidine (e.g., 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g., heteroaryl, cycloalkenyl, e.g., cycloheteroalkenyl, and the like having from 1 to 5, and particularly from 1 to 3 heteroatoms.

“Acyl” refers to a radical —C(═O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O)CH₂Ph), —C(═O)—C₁-C₈ alkyl, —C(═O)—(CH₂)ₜ(C₆-C₁₀ aryl), —C(═O)—(CH₂)ₜ(5-10 membered heteroaryl), —C(═O)—(CH₂)ₜ(C₃-C₁₀ cycloalkyl), and —C(═O)—(CH₂)ₜ(4-10 membered heterocyclyl), wherein t is an integer from 0 to 4. In certain embodiments, R²¹ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

The term aminoalkyl refers to a substituted alkyl group wherein one or more of the hydrogen atoms are independently replaced by an —NH₂ group.

The term hydroxyalkyl refers to a substituted alkyl group wherein one or more of the hydrogen atoms are independently replaced by an —OH group.

The terms “alkylamino” and “dialkylamino” refer to —NH(alkyl) and N(alkyl)₂ radicals respectively. In some embodiments the alkylamino is a —NH(C₁-C₄ alkyl). In some embodiments the alkylamino is methylamino, ethylamino, propylamino, isopropylamino, n-butylamino, iso-butylamino, sec-butylamino or tert-butylamino. In some embodiments the dialkylamino is —N(C₁-C₆ alkyl)₂. In some embodiments the dialkylamino is a dimethylamino, a methylethylamino, a diethylamino, a methylpropylamino, a methylisopropylamino, a methylbutylamino, a methylisobutylamino or a methyltertbutylamino.

The term “aryloxy” refers to an —O-aryl radical. In some embodiments the aryloxy group is phenoxy.

The term “haloalkoxy” refers to alkoxy structures that are substituted with one or more halo groups or with combinations thereof. For example, the term “fluoroalkoxy” includes haloalkoxy groups, in which the halo is fluorine. In some embodiments haloalkoxy groups are difluoromethoxy and trifluoromethoxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂N(CH₃)₂.

“Amino” refers to the radical —NH₂.

“Oxo group” refers to —C(═O)—.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

In certain embodiments, the substituent present on the nitrogen atom is a nitrogen protecting group (also referred to herein as an “amino protecting group”). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NRC)N(R^{cc})₂, —SO₂N(R^{cc})₂, —SO₂R^{cc}, —SO₂OR^{cc}, —SOR^{aa}, C(═S)N(R^{cc})₂, —C(═O)SR^{cc}, —C(═S)SR^{cc}, —C₁₋₁₀ alkyl (e.g., aralkyl, heteroaralkyl), —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference. each instance of R^{aa} is, independently, selected from —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(RC)₂, —SO₂N(R^{cc})₂, —SO₂R^{cc}, —SO₂OR^{cc}, —SOR^{aa}, —C(═S)N(R^{cc})₂, —C(═O)SRc, —C(═S)SR^{cc}, —P(═O)(R^{aa})₂, —P(═O)(OR^{cc})₂, —P(═O)(N(Re°)₂)₂, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{b} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion. each instance of R^{cc} is, independently, selected from hydrogen, —C₁₋₁₀ alkyl, —C₁₋₁₀ perhaloalkyl, —C₂₋₁₀ alkenyl, —C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{cc})₃+X-, —N(OR^{ee})R^{ff}, —SH, —SR^{ee}, —SSR^{ee}, —C(═O)R^{ee}, —CO₂H, —CO₂R^{cc}, —OC(═O)R^{ee}, —OCO₂R^{ff}, —C(═O)N(R^{ff})₂, —OC(═O)N(R^{f})₂, —NR^{ff}C(═O)R^{ee}, —NR^{ff}CO₂R^{ee}, —NRfC(═O)N(R^{ff})₂, —C(═NR^{ff})OR^{ee}, —OC(═NR^{ff})R^{ee}, —OC(═NR)OR^{ee}, —C(═NR)N(R^{ee})₂, —OC(═NR^{ee})N(RN)₂, —N^{ff}C(═NR)N(R)₂, —NR^{ff}SO₂R^{ee}, —SO₂N(R^{e})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{E})₂, —C(═O)SR^{cc} e, —C(═S)SR^{ee}, —SC(═S)SR^{ee}, —P(═O)(OR^{ee})₂—P(═O)(R^{cc})₂, —OP(═O)(R^{cc})₂, —OP(═O)(OR^{ee})₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups, or two geminal R^{d}d substituents can be joined to form ═O or ═S; wherein X⁻ is a counterion;
each instance of R^{cc} is, independently, selected from —C₁₋₆ alkyl, —C₁₋₆ perhaloalkyl, —C₂₋₆ alkenyl, —C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg}groups;
each instance of R^{f} is, independently, selected from hydrogen, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆ lkynyl, C₃₋₁₀ carbocyclyl, 3-10 membered heterocyclyl, C₆₋₁₀ aryl and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃X—, —NH(C₁₋₆ alkyl)₂+X-, —NH₂(C₁₋₆ alkyl) ⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC-6 alkyl, —Si(C₁₋₆ alkyl)₃, —OSi(C₁₋₆ alkyl)₃—C(═S)N(C₁₋₆ alkyl)₂, —C(═S)NH(C₁₋₆ alkyl), —C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, —C₁₋₆ alkyl, —C₁₋₆ perhaloalkyl, —C₂₋₆ alkenyl, —C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X—is a counterion.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitrophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Nitrogen protecting groups such as carbamate groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluorenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R)₃⁺X⁻, —P(OR^{cc})₂, —P(OR^{cc})₃X, —P(═O)(R^{aa})₂, —P(═O)(OR^{cc})₂, and —P(═O)(N(R^{bb})₂)₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, SO₂R^{aa}, —Si(R^{aa})₃, —P(RC)₂, —P(R^{cc})₃X⁻, —P(OR^{cc})₂, —P(OR^{cc})₃X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “leaving group” is given its ordinary meaning in the art of synthetic organic chemistry and refers to an atom or a group capable of being displaced by a nucleophile. Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In certain embodiments, the leaving group is halogen, alkanesulfonyloxy, arenesulfonyloxy, diazonium, alkyl diazenes, aryl diazenes, alkyl triazenes, aryl triazenes, nitro, alkyl nitrate, aryl nitrate, alkyl phosphate, aryl phosphate, alkyl carbonyl oxy, aryl carbonyl oxy, alkoxcarbonyl oxy, aryoxcarbonyl oxy ammonia, alkyl amines, aryl amines, hydroxyl group, alkyloxy group, or aryloxy. In some cases, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

“Carboxy” refers to the radical —C(═O)OH.

“Cyano” refers to the radical —CN.

“Halo” or “halogen” refers to fluoro (F), chloro (C₁), bromo (Br), and iodo (I). In certain embodiments, the halo group is either fluoro or chloro.

“Haloalkyl” refers to an alkyl radical in which the alkyl group is substituted with one or more halogens. Typical haloalkyl groups include, but are not limited to, trifluoromethyl (—CF₃), difluoromethyl (—CHF₂), fluoromethyl (—CH₂F), chloromethyl (—CH₂Cl), dichloromethyl (—CHCl₂), tribromomethyl (—CH₂Br), and the like.

“Hydroxy” refers to the radical —OH.

“Nitro” refers to the radical —NO₂.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. Any and all such combinations are contemplated in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —N₀₂, —N₃, —SO₂H, —SO₃H, —OH, —OR^{aa}, —ON(R^{bb})₂, —N(R^{bb})₂, —N(R^{bb})₃+X⁻, —N(OR^{cc})R^{bb}, SH, —SR^{aa}, —SSR^{cc}, —C(═O)R^{aa}, —CO₂H, —CHO, —C(OR^{cc})₂, —CO₂R^{aa}, —OC(═O)R^{aa}, —0.0 R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —S(═O)(═NRb)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NRbP(═O)(NRb)₂, —P(R^{cc})₂—P(R^{cc})₃, —OP(R^{cc})₂—OP(R^{cc})₃, —B(R^{aa})₂, —B(OR^{cc})₂, -BRaa(OR^{cc}), C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R,d groups; or two geminal hydrogens on a carbon atom are replaced with the group ═O, ═S, ═NN(R^{bb})₂, ═NNR^{ee}bC(═O)R^{aa}, ═NNR^{ee}bC(═O)OR^{aa}, ═NNRbS(═O)₂R^{aa}, ═NR^{bb}, or ═NORc; each instance of R^{aa} is, independently, selected from C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd}groups;
each instance of R^{cc} is, independently, selected from hydrogen, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{cc}, —ON(R^{E})₂, —N(R^{E})₂, —N(R)₃⁺X⁻, —N(OR^{ee})R^{ee}, —SH, —SR^{ee}, —SSR^{ee}—C(═O)R^{cc}, —CO₂H, —CO₂R^{ee}, —OC(═O)R^{ee}, —OCO₂R^{ee}, —C(═O)N(Re)₂, —OC(═O)N(R^{E})₂, —NR^{ee}C(═O)R^{cc} e, —NR^{ee}CO₂R^{ee}, —NR^{ee}C(═O)N(RE)₂, —C(═NR^{ee})OR^{cc} e, —OC(═NR^{ee})R^{ee}, —OC(═NR^{ff})OR^{cc} e, —C(═NRN)N(RK)₂, —OC(═NRN)N(RE)₂, —NR^{ee}C(═NR^{ff})N(R^{ff})₂, —NR^{ee}SO₂R^{ee}, —SO₂N(R^{K})₂, —SO₂R^{ee}, —SO₂OR^{ee}, —OSO₂R^{ee}, —S(═O)R^{ee}, —Si(Re)₃, —OSi(R^{ee})₃, —C(═S)N(RE)₂, —C(═O)SR^{ee}, —C(═S)SR^{ee}, —SC(═S)SR^{ee}, —P(═O)₂R^{ee}, —P(═O)(R^{ee}) —OP(=O)(R^{ee})₂, —OP(═O)(OR^{ee})₂, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of Rn is, independently, selected from hydrogen, C₁₋₆ alkyl, C₁₋₆ haloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg}groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)+X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —C₀₂(C₁₋₆ alkyl), —OC(═O)(C₁₋₆ alkyl), —OC₀₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-membered heterocyclyl, 5-10 membered heteroaryl; or two geminal R⁹⁹ substituents can be joined to form ═O or ═S; wherein X is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —S₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above. These and other exemplary substituents are described in more detail in the Detailed Description, Examples, and Claims. The invention is not intended to be limited in any manner by the above exemplary listing of substituents.

### Other Definitions

As used herein, the term “salt” refers to any and all salts and encompasses pharmaceutically acceptable salts.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomologus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

In general, the “effective amount” of a compound refers to an amount sufficient to elicit the desired biological response e.g., to treat a disease or disorder described herein. As will be appreciated by those of ordinary skill in this art, the effective amount of a compound of the disclosure may vary depending on such factors as the desired biological endpoint, the pharmacokinetics of the compound, the disease being treated, the mode of administration, and the age, health, and condition of the subject. An effective amount encompasses therapeutic and prophylactic treatment (i.e., encompasses a “therapeutically effective amount” and a “prophylactically effective amount”).

As used herein, and unless otherwise specified, a “therapeutically effective amount” of a compound is an amount sufficient to provide a therapeutic benefit in the therapeutic treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the therapeutic treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

### Compounds

Provided herein are compounds of Formula (I) or pharmaceutically acceptable salts thereof. Unless the context requires otherwise, reference throughout this specification to “a compound of Formula (I)” or “compounds of Formula (I)” refers to all embodiments of Formula (I), including, for example, compounds of Formula (I), Formula (Ia), Formula (Ib), Formula (Ic), Formula (Id), Formula (II), Formula (IIa), Formula (IIb), Formula (IIc), Formula (IId), Formula (III), Formula (IIIa), Formula (IIIb), Formula (IIIc), Formula (IIId), Formula (I_1), Formula (I_1a), Formula (I_2), Formula (I_2a), Formula (I_3), Formula (I_3a), Formula (I_4), Formula (I_4a), Formula (I_5), Formula (I_5a), Formula (I_6), Formula (I_6a), Formula (I_7), Formula (I_7a), Formula (I_8), Formula (I_8a), Formula (19), Formula (I_9a), Formula (III), Formula (I_1a), Formula (I_1_2), Formula (II_2a), Formula (I_13), Formula (I_13a), Formula (I_1_4), Formula (I_1_4a), Formula (I_15), Formula (II_5a), Formula (I_16), Formula (I_16a), Formula (I_17), Formula (I_17a), Formula (I_18), Formula (I_18a), Formula (I_19), Formula (I_19a), Formula (III_1), Formula (III_1a), Formula (III_2), Formula (III_2a), Formula (I_113), Formula (III_3a), Formula (III_4), Formula (III_4a), Formula (I_115), Formula (III_5a), Formula (I_116), Formula (III_6a), Formula (I_117), Formula (III_7a), Formula (I_118), Formula (III_8a), Formula (I_119), Formula (III_9a), Formula (IV), Formula (IVa), Formula (IV_1), Formula (IV_1a), Formula (IV_2), Formula (IV_2a), Formula (IV_3), Formula (IV_3a), Formula (IV_4), Formula (IV_4a), Formula (IV_5), Formula (IV_5a), Formula (V_1), Formula (V_1a), Formula (V_2), Formula (V_2a), Formula (VI), Formula (VIa), Formula (VI_1), Formula (VI_1a), (i.e., Formula (I)-Formula (VI_1a) as well as the compounds of Table 1.

In one embodiment, provided herein are compounds of Formula (I):

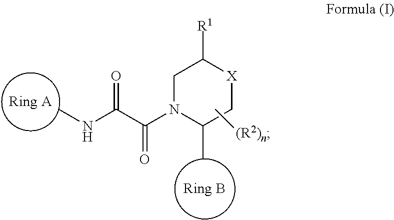

or pharmaceutically acceptable salts thereof, wherein:
X is selected from the group consisting of the group consisting of —O— and —NR⁷—;
Ring A is selected from the group consisting of an optionally substituted fused bicyclic 8-10 membered heteroaryl ring system containing at least one nitrogen atom, wherein the 8-10 membered refers to the total number of atoms in the fused system and optionally substituted pyridin-3-yl;
Ring B is selected from the group consisting of C₆-C₁₀ aryl and 5-10 membered heteroaryl, each optionally substituted at any available position;
each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1}and —S(═O)₂N(Ra);
each R² is independently selected from the group consisting of H, -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring);
each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂, wherein each alkyl, cycloalkyl, heterocyclyl, aryl, heteroaryl, arylalkyl and heteroarylalkyl is optionally substituted at any available position;
each R^{a1}, R^{a2} and R^{a7} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(=)OR, —NR^{b}C(═O)R^{cc}, —NR^{b}C(═O)OR, —C(═O)N(R^{b})₂, —OC(═O)N(R^{b})₂, —S(═O)R^{cc}, —S(═O)₂R^{b}, —SR^{b}, —S(=O)(═NR^{b})R, —NR^{b}S(═O)₂R and —S(═O)₂N(R^{b})₂, wherein each R is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl); and
n is 0, 1, 2 or 3.

In one embodiment, provided is a compound of Formula (I):

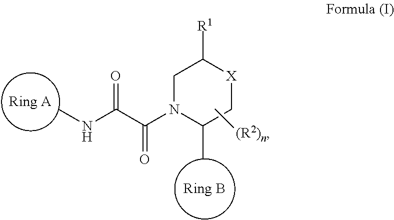

or a pharmaceutically acceptable salt thereof,
wherein:
X is selected from the group consisting of the group consisting of —O— and —NR⁷—;
Ring A is selected from the group consisting of an optionally substituted fused bicyclic 8-10 membered heteroaryl ring system containing at least one nitrogen atom, wherein the 8-10 membered refers to the total number of atoms in the fused system and pyridin-3-yl, wherein the 8-10 membered heteroaryl and pyridin-3-yl are substituted at any available positions with 0, 1, 2, 3 or 4 instances of R⁴;
Ring B is selected from the group consisting of C₆-C₁₀ aryl and 5-10 membered heteroaryl, each substituted at any available position with 0, 1, 2 or 3 instances of R³;
each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1}and —S(=O)₂N(R^{a1})₂;
each R² is independently selected from the group consisting of H, -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring);
each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂, wherein each alkyl, cycloalkyl, heterocyclyl, aryl, heteroaryl, arylalkyl and heteroarylalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof.
each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR³C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein each alkyl, cycloalkyl, heteroalkyl, heterocyclyl, aryl, heteroaryl, cycloalkylalkyl, heterocyclylalkyl, arylalkyl and heteroarylalkyl of R³ is substituted at any available position with 0, 1, 2 or 3 instances of -Me, —OH, —C(═O)CH₃, —C(═O)NHCH₃, —NH₂, —NHC(═O)CH₃ or a combination thereof,
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, NR^{a4}S(═O)₂R^{a4} and —S(═O)₂N(R^{a4})₂;
each R^{a1}, R^{a2}, R^{a3}, R^{a4} and R^{a7} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is substituted at any available position with 0, 1, 2 or 3 instances of R⁵, wherein each R is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R, —C(═O)OR^{cc}, —NR^{b}C(═O)R^{b}, —NR^{b}C(═O)R, —C(═O)N(R^{b})₂, —OC(═O)N(R^{b})₂, —S(═O)R^{b}, —S(═O)₂R, —SR^{b}, —S(═O)(═NR^{b})R, —NR^{b}S(═O)₂R^{b} and —S(═O)₂N(R^{b})₂, wherein each R is independently selected from the group consisting of H, —C₁-C₆ alkyl (e.g., -Me, -Et, —Pr, —ⁱPr, -^{tt}Bu, -^{t}Bu, -sec-Bu, -iso-Bu)), and C₃-C₉ cycloalkyl (e.g., cyclopropyl, cyclobutyl, cyclopentyl, cyclohexyl); and
n is 0, 1, 2 or 3.

In some embodiments, R¹ is absent (e.g., if a spiro ring formed by two R² groups is attached to the atom that would otherwise bear R¹).

In certain embodiments, R¹ is not absent or H and Ring B and R¹ are in a trans relative configuration. In other embodiments, R¹ is not absent or H and Ring B and R¹ are in a cis relative configuration.

In some embodiments, the moiety represented as

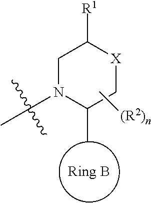

in Formula (I) is selected from the group consisting of:

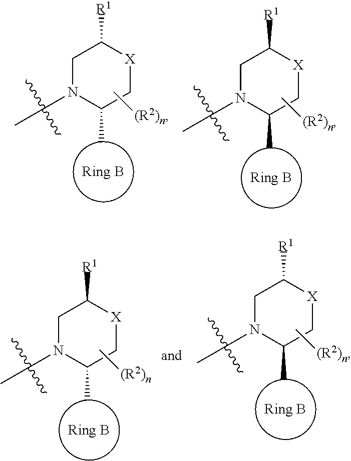

wherein X, Ring B, R¹, R² and n are as defined in any of the embodiments described herein.

In certain embodiments, the moiety represented as

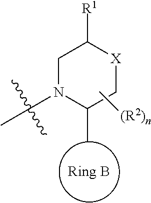

is selected from the group consisting of:

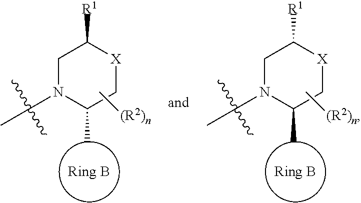

wherein X, Ring B, R¹, R² and n are as defined in any of the embodiments described herein.

In other embodiments, the moiety represented as

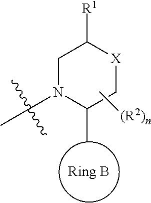

is selected from the group consisting of:

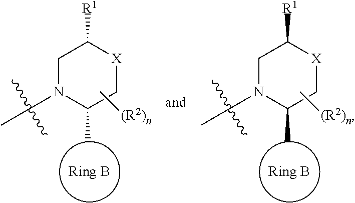

wherein X, Ring B, R¹, R² and n are as defined in any of the embodiments described herein.

In some embodiments, the compound of Formula (I) is of Formula (Ia)

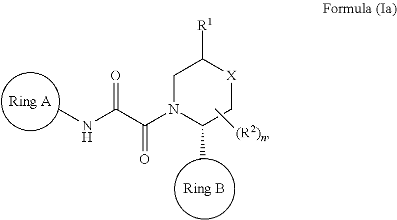

wherein X, Ring A, Ring B, R¹, R² and n are as defined in any of the embodiments described herein.

In some embodiments, the compound of Formula (I) is of Formula (Ib)

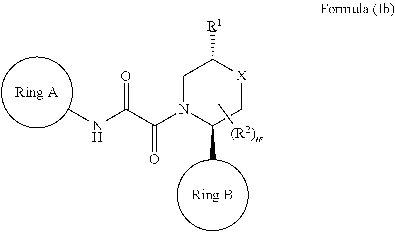

wherein X, Ring A, Ring B, R¹, R² and n are as defined in any of the embodiments described herein.

In some embodiments, the compound of Formula (I) is of Formula (Ic)

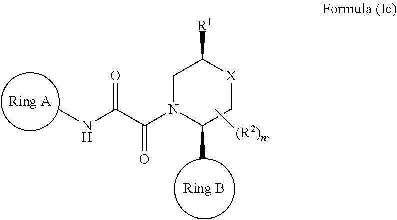

wherein X, Ring A, Ring B, R¹, R² and n are as defined in any of the embodiments described herein.

In some embodiments, the compound of Formula (I) is of Formula (Id)

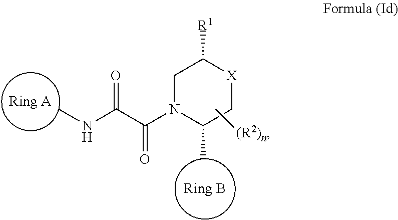

wherein X, Ring A, Ring B, R^{cc}, R² and n are as defined in any of the embodiments described herein.

As generally defined herein, X is selected from the group consisting of —O— and —NR⁷—.

In some embodiments, X is —O—.

In some embodiments, the compound of Formula (I) is of Formula (II):

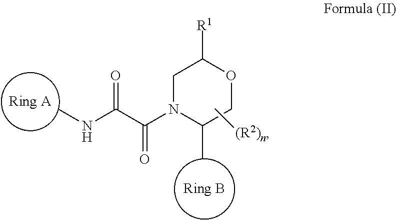

wherein Ring A, Ring B, R¹, R² and n are as defined in any of the embodiments described herein.

In some embodiments, the compound of Formula (I) is of Formula (IIa):

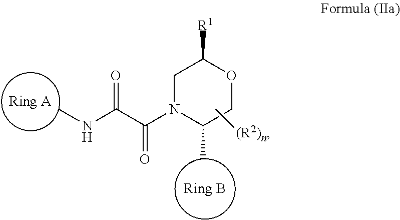

wherein Ring A, Ring B, R¹, R² and n are as defined in any of the embodiments described herein.

In some embodiments, the compound of Formula (I) is of Formula (IIb):

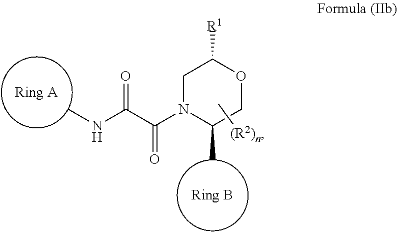

wherein Ring A, Ring B, R¹, R² and n are as defined in any of the embodiments described herein.

In some embodiments, the compound of Formula (I) is of Formula (IIc):

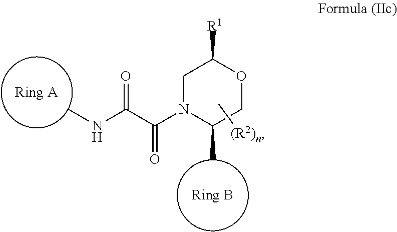

wherein Ring A, Ring B, R¹, R² and n are as defined in any of the embodiments described herein.

In some embodiments, the compound of Formula (I) is of Formula (IId):

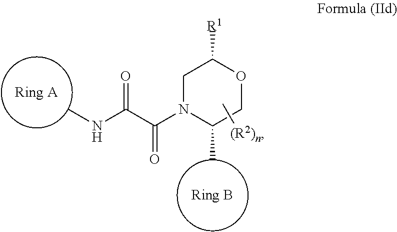

wherein Ring A, Ring B, R¹, R² and n are as defined in any of the embodiments described herein.

In some embodiments, the compound is of Formula (IIa) or Formula (IIb).

In some embodiments, the compound is of Formula (IIc) or Formula (IId).

In some embodiments, X is —NR⁷—, wherein R⁷ is as defined in any of the embodiments described herein.

In some embodiments, the compound of Formula (I) is of Formula (III):

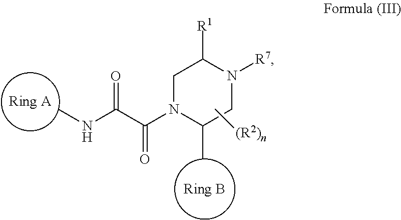

wherein Ring A, Ring B, R¹, R², R⁷ and n are as defined in any of the embodiments described herein.

In some embodiments, the compound of Formula (I) is of Formula (IIIa):

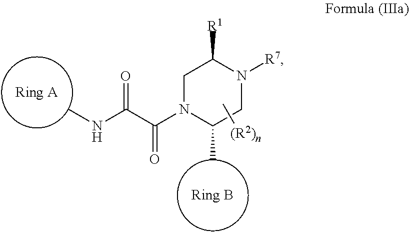

wherein Ring A, Ring B, R¹, R², R⁷ and n are as defined in any of the embodiments described herein.

In some embodiments, the compound of Formula (I) is of Formula (IIIb):

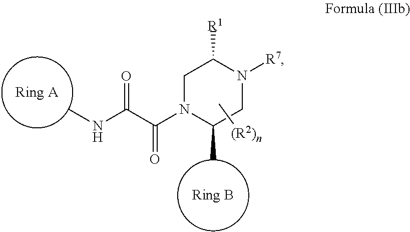

wherein Ring A, Ring B, R¹, R², R⁷ and n are as defined in any of the embodiments described herein.

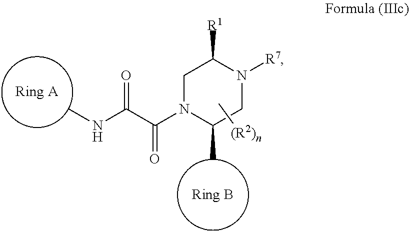

In some embodiments, the compound of Formula (I) is of Formula (IIId):

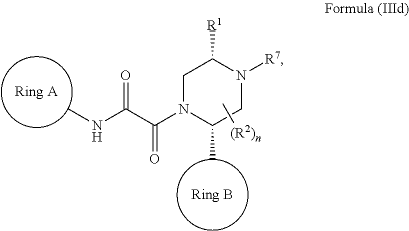

wherein Ring A, Ring B, R¹, R², R⁷ and n are as defined in any of the embodiments described herein.

In some embodiments, the compound is of Formula (IIIa) or Formula (IIIb).

In some embodiments, the compound is of Formula (IIIc) or Formula (IIId).

As generally defined herein, Ring A is an optionally substituted fused bicyclic 8-10 membered heteroaryl ring system containing at least one nitrogen atom, wherein the 8-10 membered refers to the total number of atoms in the fused system or pyridin-3-yl.

In some embodiments, Ring A is a fused bicyclic 8-10 membered heteroaryl ring containing at least one nitrogen atom, wherein the 8-10 membered refers to the total number of atoms in the fused system or pyridin-3-yl, wherein Ring A is substituted at any available positions with 0, 1, 2, 3 or 4 instances of R⁴, wherein R⁴ is as defined herein.

In some embodiments, Ring A is an optionally substituted fused bicyclic 8-10 membered heteroaryl ring system containing at least one nitrogen atom and 0, 1, 2 or 3 additional heteroatoms selected from the group consisting of N, O and S or oxidized forms thereof, wherein the 8-10 membered refers to the total number of atoms in the fused system.

In some embodiments, Ring A is substituted at any available positions with 0, 1, 2 or 3 instances of R⁴, wherein R⁴ is as defined herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a carbocyclyl or heterocyclyl ring, wherein the total number of atoms in the fused system is between 8 and 10 and the system contains a total of 2, 3 or 4 heteroatoms selected from the group consisting of N, O and S or oxidized forms thereof. The attachment point is on the heteroaryl ring.

In some embodiments, Ring A is substituted at any available positions with 0, 1, 2 or 3 instances of R⁴, wherein R⁴ is as defined herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a carbocyclyl ring, wherein the total number of atoms in the fused system is between 8 and 10.

In some embodiments, Ring A is substituted at any available positions with 0, 1, 2 or 3 instances of R⁴, wherein R⁴ is as defined herein.

In some embodiments, Ring A contains a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), fused to a heterocyclyl ring, wherein the total number of atoms in the fused system is between 8 and 10 and the system contains a total of 2, 3 or 4 heteroatoms selected from the group consisting of N, O and S or oxidized forms thereof. In some embodiments, Ring A is substituted at any available positions with 0, 1, 2 or 3 instances of R⁴, wherein R⁴ is as defined herein.

In some embodiments, Ring A contains a phenyl ring fused with a 5-6 membered monocyclic heteroaryl ring containing at least one nitrogen atom and 0, 1 or 2 additional heteroatoms selected from the group consisting of N, O or S or oxidized forms thereof (e.g., pyridyl, pyrimidinyl, pyrazinyl, pyridazinyl, pyrrolyl, imidazolyl, thiazolyl, oxazolyl, oxadiazolyl, oxathiazolyl), wherein the attachment point is on either the phenyl or the heteroaryl ring. In some embodiments, Ring A is substituted at any available positions with 0, 1, 2 or 3 instances of R⁴, wherein R⁴ is as defined herein.

In some embodiments, Ring A is optionally substituted pyridin-3-yl. In some embodiments, Ring A is pyridin-3-yl, substituted at any available positions with 0, 1, 2, 3 or 4 instances of R⁴, wherein R⁴ is as defined herein

In some embodiments, Ring A is selected from the group consisting of

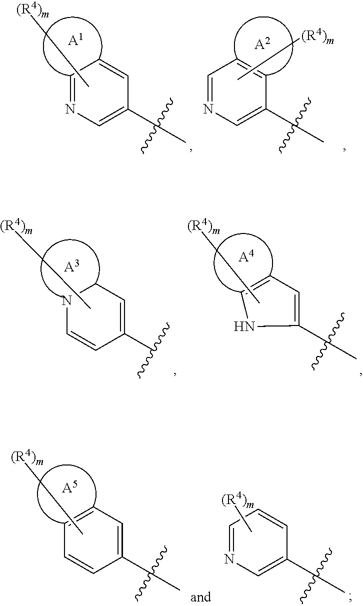

wherein
each of rings A¹, A² and A⁴ is independently 4-6 membered carbocyclyl, 4-6 membered heterocyclyl, 5-6 membered heteroaryl or phenyl;
each ring A³ is independently a 4-6 membered heterocyclyl or 5-6 membered heteroaryl, wherein the heterocyclyl and heteroaryl contain at least one nitrogen atom each ring A⁵ is independently a 5-6 membered heteroaryl, wherein the heteroaryl contains at least one nitrogen atom;
each R⁴ and m are as defined in any of the embodiments described herein.

In one embodiment, rings A¹, A², and A⁴ are each independently a 4-6 membered carbocyclyl, a 4-6 membered heterocyclyl containing 1, 2 or 3 heteroatoms selected from the group consisting of O, N, S or oxidized forms thereof, a 5-6 membered heteroaryl containing 1, 2 or 3 heteroatoms selected from the group consisting of O, N, S or oxidized forms thereof or a phenyl.

In one embodiment, each ring A³ is independently a 4-6 membered heterocyclyl or 5-6 membered heteroaryl, wherein the heterocyclyl and heteroaryl contain at least one nitrogen atom and 0, 1 or 2 additional heteroatoms selected from the group consisting of N, O or S or oxidized forms thereof.

In one embodiment, each ring A⁵ is independently a 5-6 membered heteroaryl, wherein the heteroaryl contains at least one nitrogen atom and 0, 1 or 2 additional heteroatoms selected from the group consisting of N, O or S or oxidized forms thereof.

In one embodiment, Ring A is

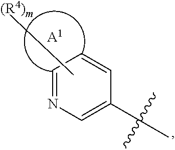

wherein A¹, R⁴ and m are as defined herein.

In one embodiment, ring A is

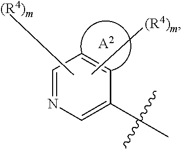

wherein A², R⁴ and m are as defined herein.

In one embodiment, ring A is

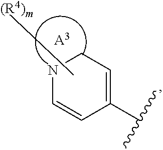

wherein A³, R⁴ and m are as defined herein.

In one embodiment, ring A is

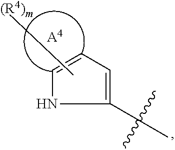

wherein A⁴, R⁴ and m are as defined herein.

In one embodiment, ring A is

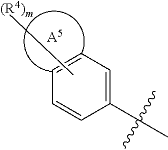

wherein A⁵, R⁴ and m are as defined herein.

In some embodiments, Ring A is selected from the group consisting of:

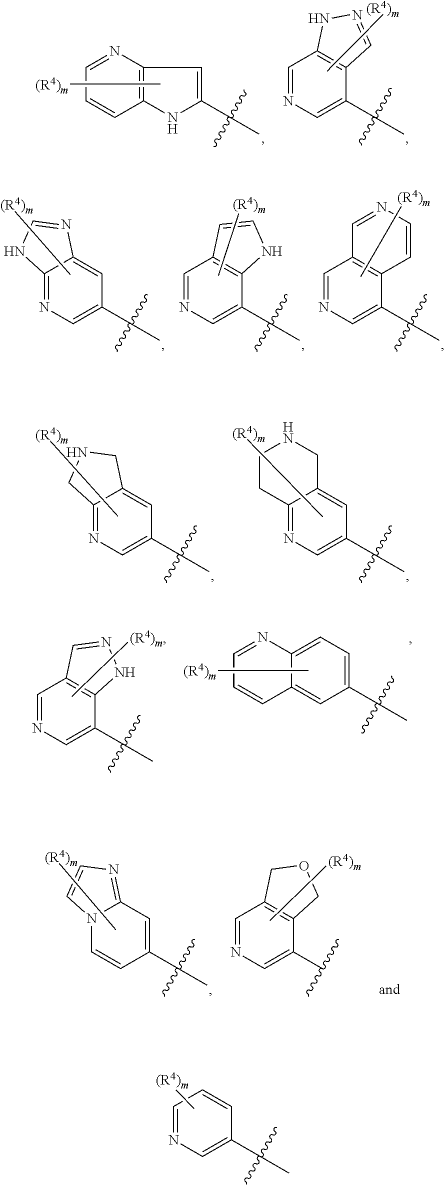

wherein R⁴ and m are as defined herein.

In some embodiments, Ring A is selected from the group consisting of:

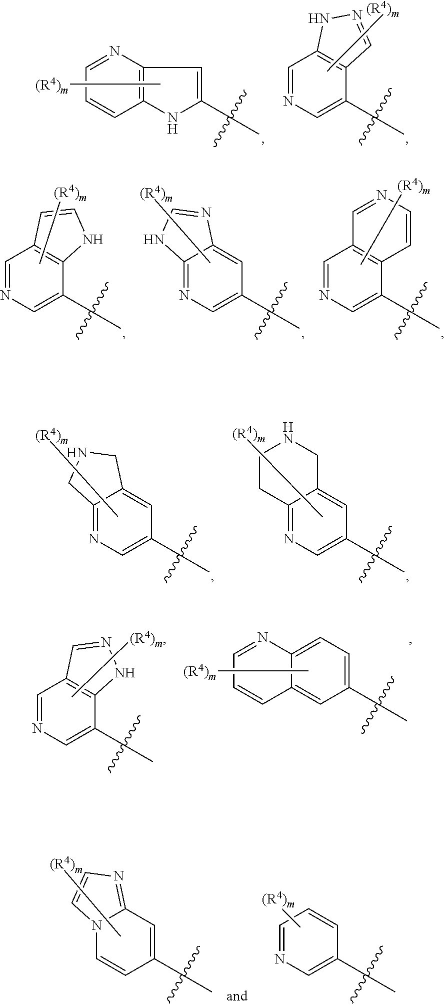

wherein R⁴ and m are as defined herein.

In some embodiments, Ring A is selected from the group consisting of:

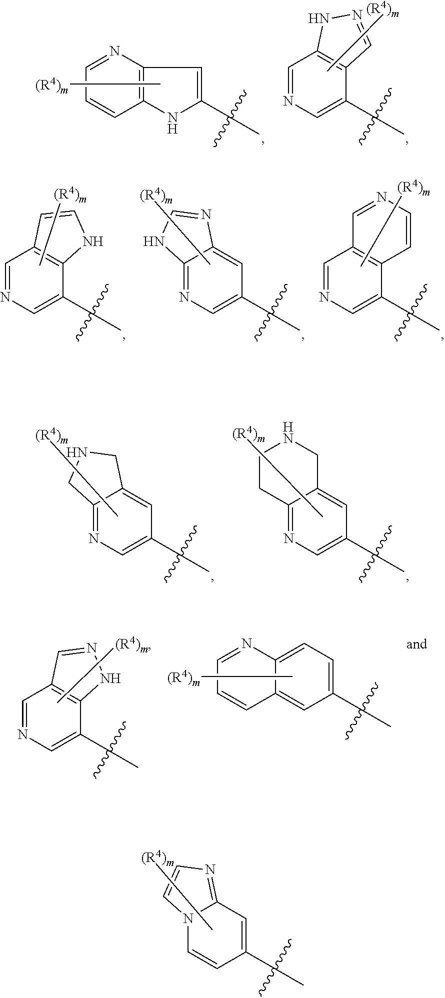

wherein R⁴ and m are as defined herein.

In some embodiments some embodiments, Ring A is selected from the group consisting of:

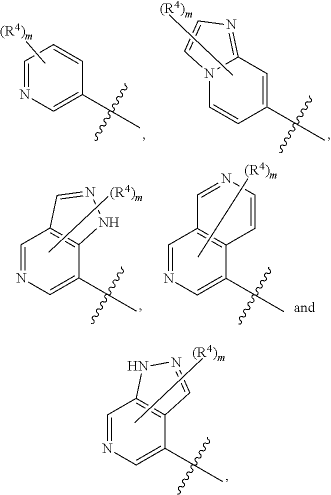

wherein R⁴ and m are as defined herein.

In some embodiments, Ring A is selected from the group consisting of:

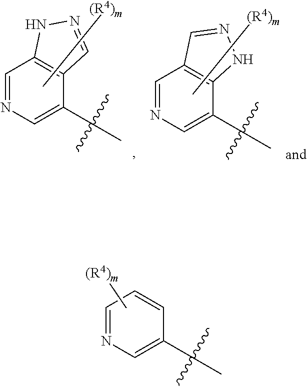

wherein R⁴ and m are as defined herein.

In some embodiments some embodiments, Ring A is selected from the group consisting of:

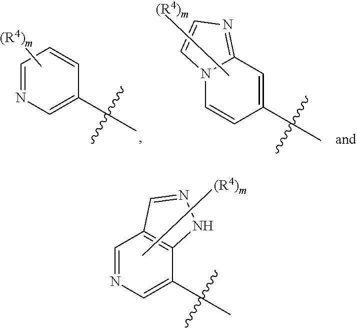

wherein R⁴ and m are as defined herein.

In some embodiments some embodiments, Ring A is selected from the group consisting of:

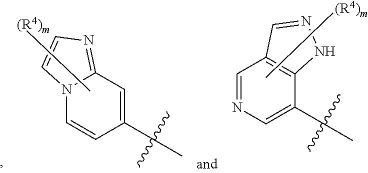

wherein R⁴ and m are as defined herein.

In some embodiments some embodiments, Ring A is selected from the group consisting of:

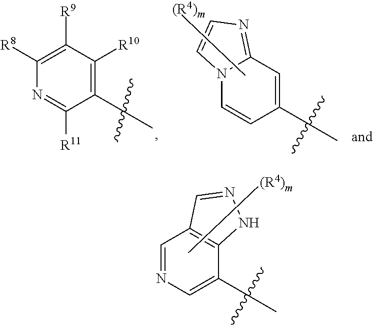

wherein R⁴ R⁸, R⁹, R¹⁰, R¹¹ and m are as defined herein. In some embodiments m is 0, 1, 2 or 3. In some embodiments m is 0, 1 or 2. In some embodiments, m is 0 or 1.

In some embodiments some embodiments, Ring A is selected from the group consisting of:

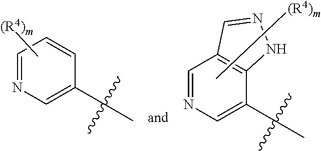

wherein R⁴ and m are as defined herein.

In some embodiments some embodiments, Ring A is selected from the group consisting of:

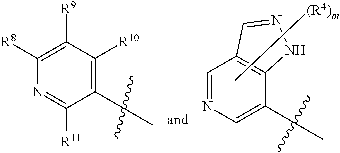

wherein R⁴ R⁸, R⁹, R¹⁰, R¹¹ and m are as defined herein. In some embodiments m is 0, 1, 2 or 3. In some embodiments m is 0, 1 or 2. In some embodiments, m is 0 or 1.

In some embodiments, ring A is:

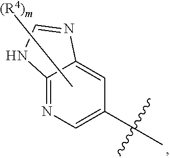

wherein R⁴ and m are as defined herein.

In some embodiments, ring A is:

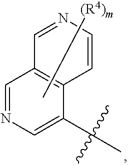

wherein R⁴ and m are as defined herein.

In some embodiments, ring A is:

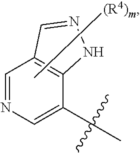

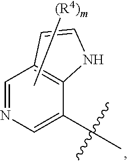

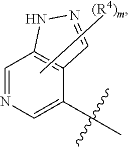

wherein R⁴ and m are as defined herein.

In some embodiments, ring A is:

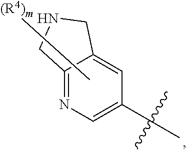

wherein R⁴ and m are as defined herein.

In some embodiments, ring A is:

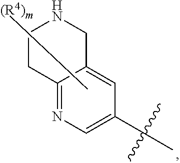

wherein R⁴ and m are as defined herein.

In some embodiments, ring A is:

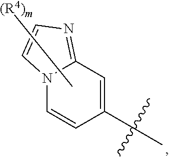

wherein R⁴ and m are as defined herein.

In some embodiments, ring A is:

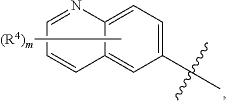

wherein R⁴ and m are as defined herein.

In some embodiments, ring A is:

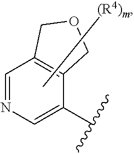

wherein R⁴ and m are as defined herein.

In some embodiments, ring A is:

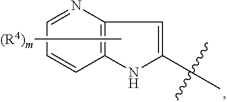

wherein R⁴ and m are as defined herein.

In one embodiment, ring A is

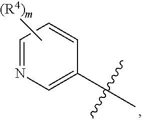

wherein R⁴ and m are as defined herein.

In some embodiments some embodiments, Ring A is:

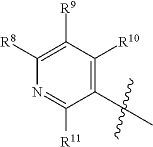

wherein R⁸, R⁹, R¹⁰ and are as defined herein.

As generally defined herein, m is 0, 1, 2, 3 or 4.

In some embodiments, m is 0, 1, 2 or 3. In some embodiments, m is 0, 1 or 2.

In some embodiments, m is 0 or 1. In some embodiments, m is 1 or 2.

In some embodiments, m is 0.

In some embodiments, m is 1.

In some embodiments, m is 2.

In some embodiments, m is 3.

In some embodiments, m is 4.

In some embodiments, Ring A is selected from the group consisting of:

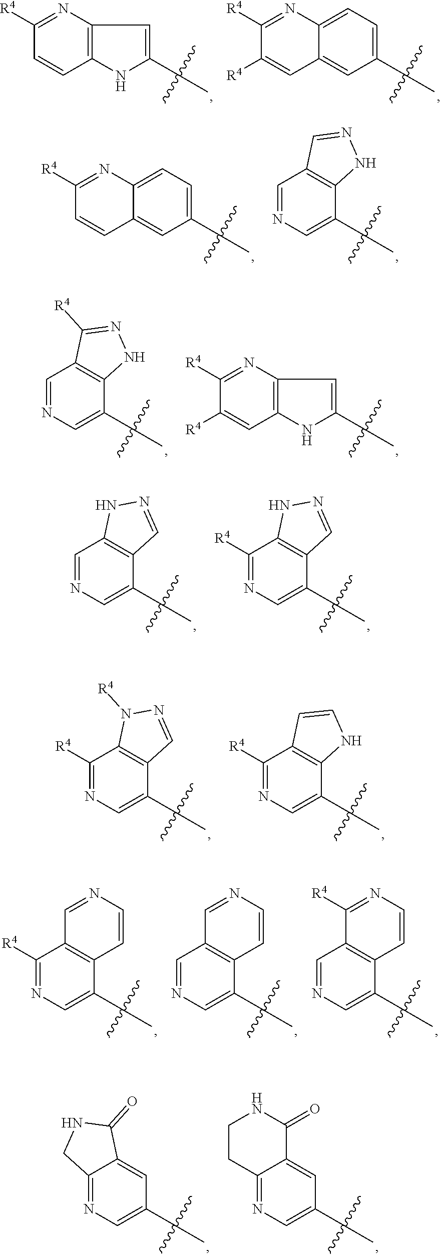

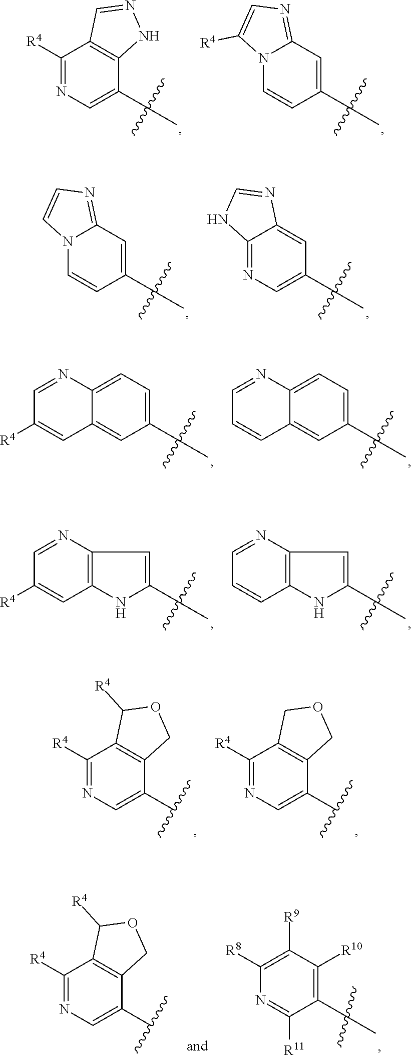

wherein R⁴, R⁸, R⁹, R¹⁰ and R¹¹ are as defined in any of the embodiments described.

In some embodiments, Ring A is selected from the group consisting of:

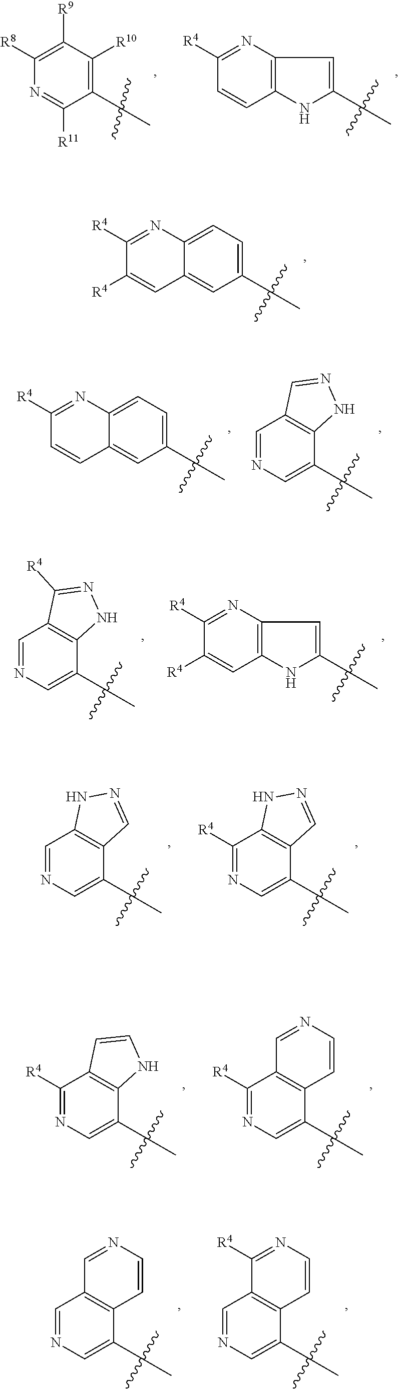

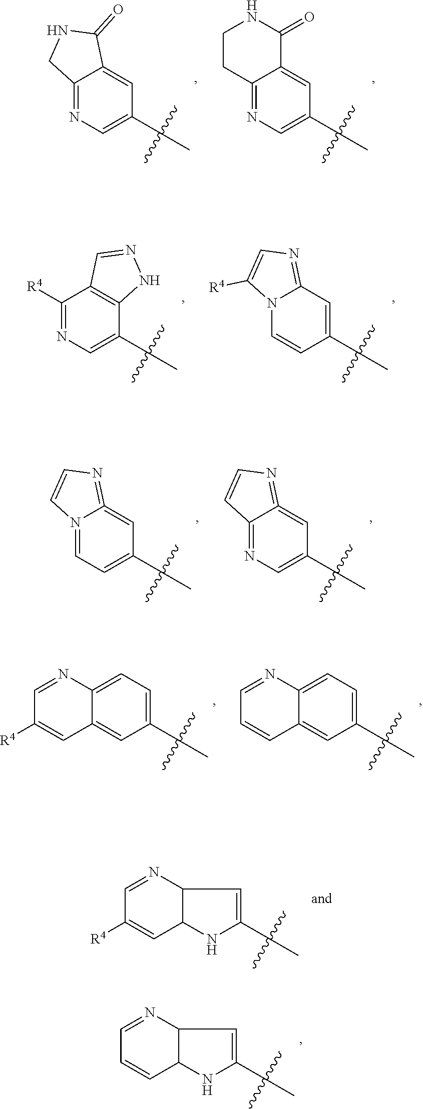

wherein R⁴, R⁸, R⁹, R¹⁰ and R¹¹ are as defined in any of the embodiments described herein.

In some embodiments, Ring A is selected from the group consisting of:

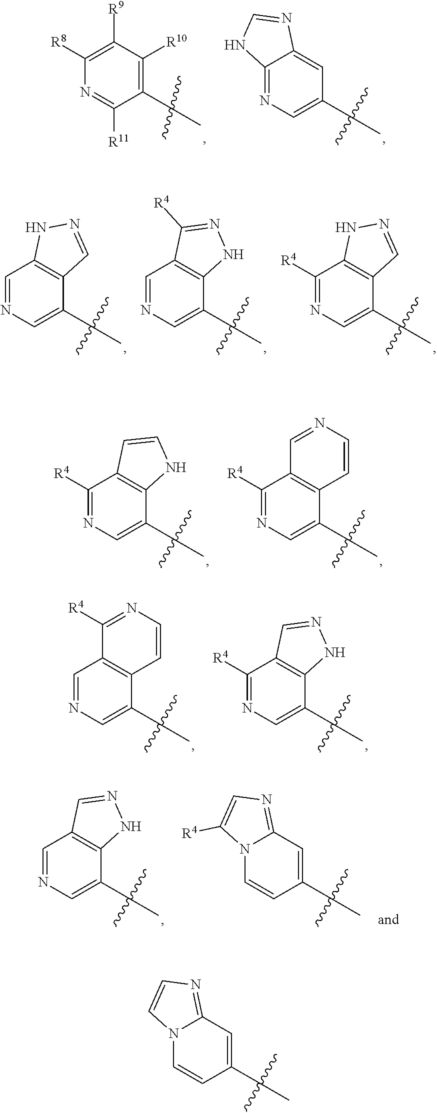

wherein R⁴ R⁸ R⁹, R¹⁰ and R¹¹ are as defined in any of the embodiments described herein.

In some embodiments, Ring A is selected from the group consisting of:

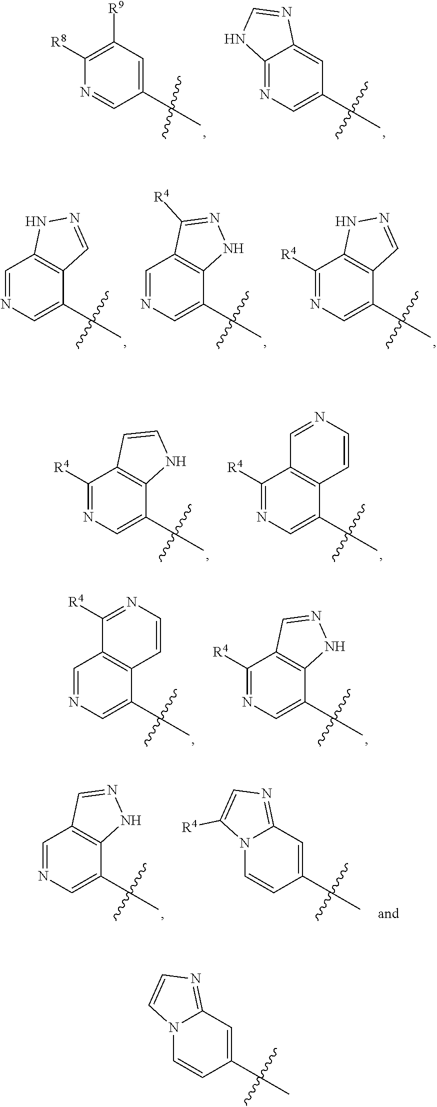

wherein R⁴, R⁸ and R⁹ are as defined in any of the embodiments described herein.

In some embodiments, Ring A is selected from the group consisting of:

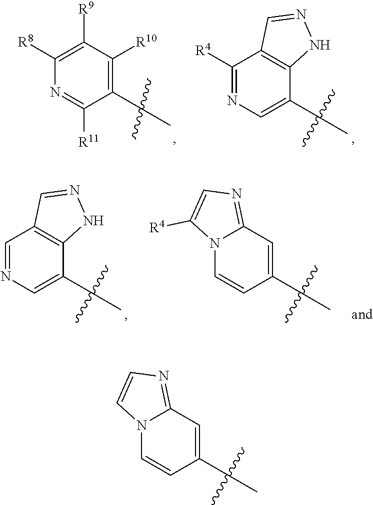

wherein R⁴, R⁸, R⁹, R¹⁰ and R¹¹ are as defined in any of the embodiments described herein.

In some embodiments, Ring A is selected from the group consisting of:

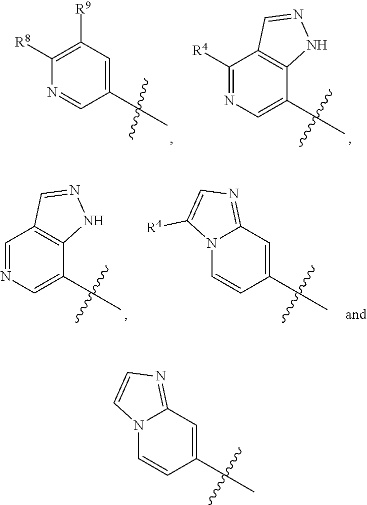

wherein R⁴, R⁸ and R⁹ are as defined in any of the embodiments described herein.

In some embodiments, Ring A is selected from the group consisting of:

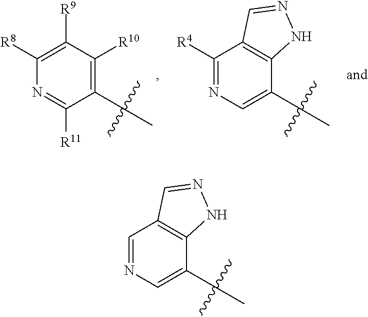

wherein R⁴, R⁸, R⁹, R⁵⁰ and R¹¹ are as defined in any of the embodiments described herein.

In some embodiments, Ring A is selected from the group consisting of:

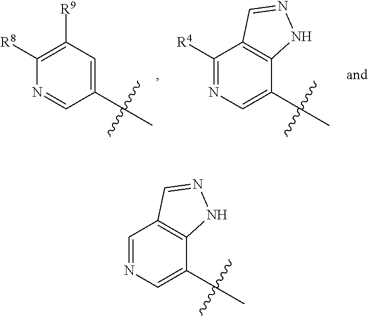

wherein R⁴, R⁸ and R⁹ are as defined in any of the embodiments described herein.

In some embodiments, Ring A is selected from the group consisting of:

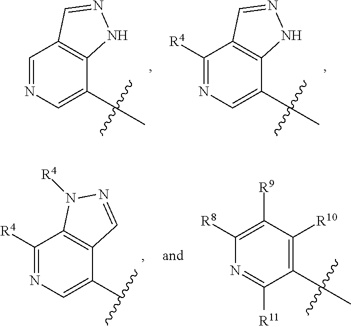

wherein R⁴, R⁸, R⁹, R¹° and R¹¹ are as defined in any of the embodiments described herein.

In some embodiments, Ring A is selected from the group consisting of:

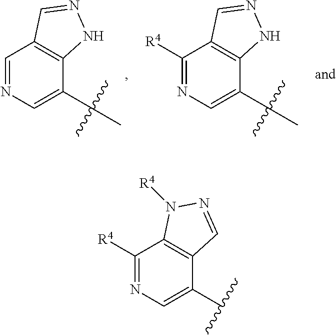

wherein R⁴ is as defined in any of the embodiments described herein.

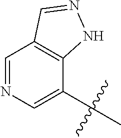 [SMILES: *c1cncc2cn[nH]c12]

In some embodiments, Ring A is

In some embodiments, Ring A is

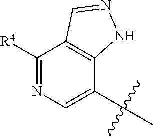

wherein R⁴ is as defined in any of the embodiments described herein.

In some embodiments, Ring A is

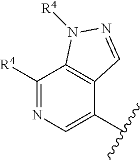

wherein R⁴ is as defined in any of the embodiments described herein.

In some embodiments, Ring A is

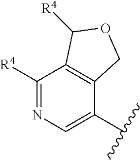

wherein R⁴ is as defined in any of the embodiments described herein.

In some embodiments, Ring A is

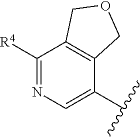

wherein R⁴ is as defined in any of the embodiments described herein.

In some embodiments, Ring A is selected from the group consisting of:

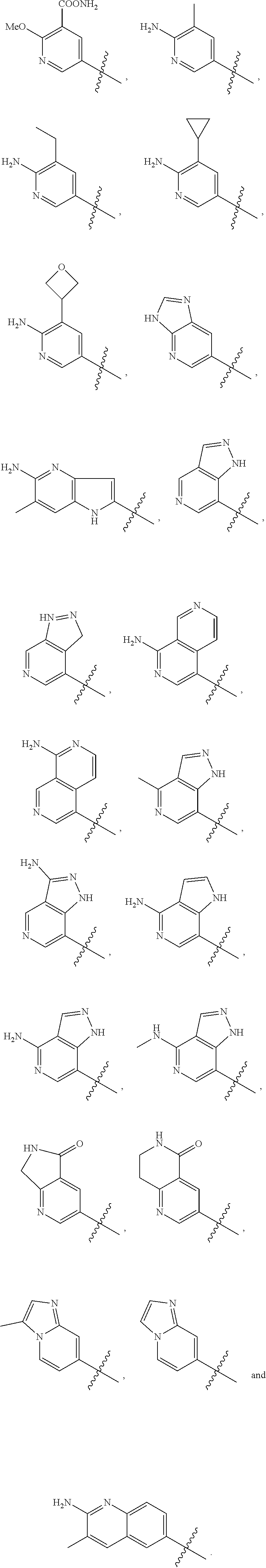

In some embodiments, Ring A is selected from the group consisting of:

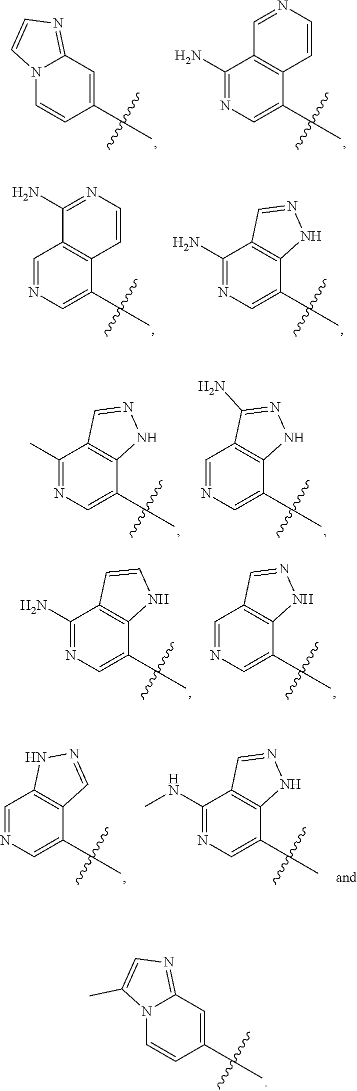

In some embodiments, Ring A is selected from the group consisting of:

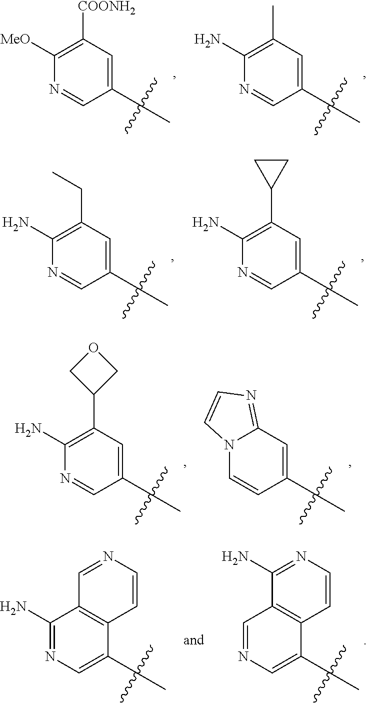

In some embodiments, Ring A is selected from the group consisting of:

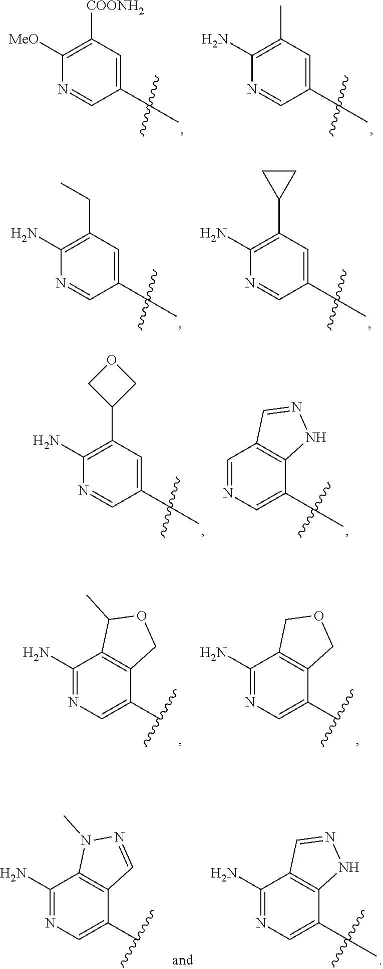

In some embodiments, Ring A is selected from the group consisting of:

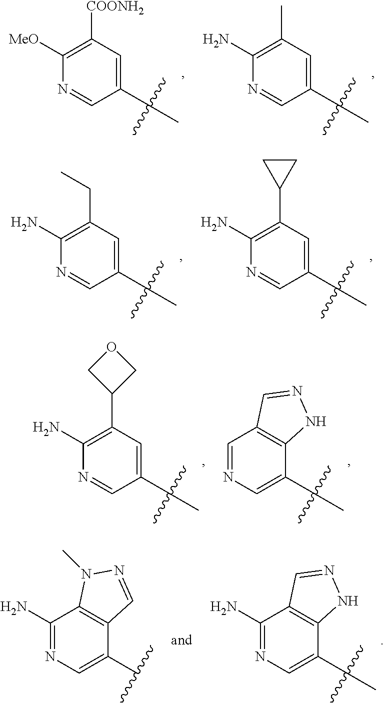

In some embodiments, Ring A is selected from the group consisting of:

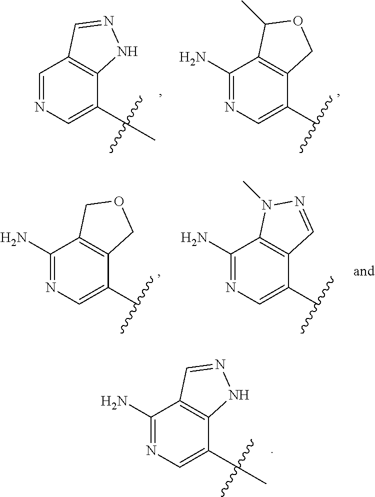 [SMILES: *c1cncc2cn[nH]c12 ; *c1cnc(N)c2c1COC2C ; *c1cnc(N)c2c1COC2 ; *c1cnc(N)c2c1cnn2C ; *c1cnc(N)c2cn[nH]c12]

In some embodiments, Ring A is selected from the group consisting of:

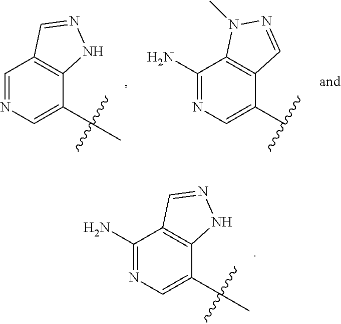 [SMILES: *c1cncc2cn[nH]c12 ; *c1cnc(N)c2c1cnn2C ; *c1cnc(N)c2cn[nH]c12]

In some embodiments, Ring A is selected from the group consisting of:

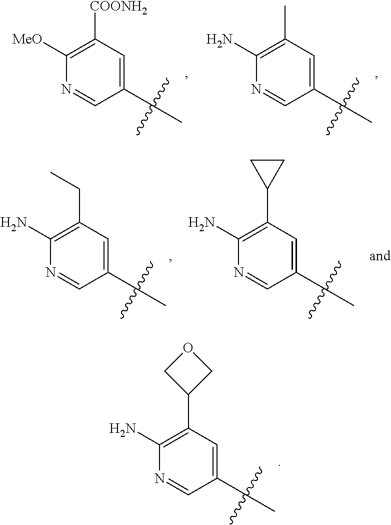

In certain embodiments, Ring A is selected from the group consisting of:

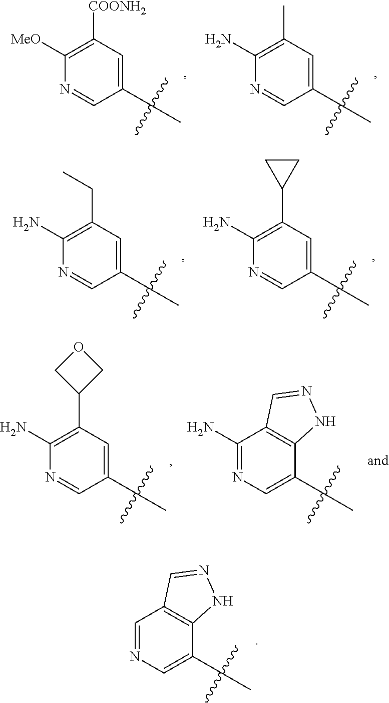

In some embodiments, Ring A is:

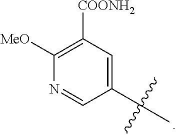

In some embodiments, Ring A is:

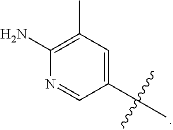 [SMILES: *c1cnc(N)c(C)c1]

In some embodiments, Ring A is:

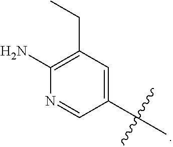 [SMILES: *c1cnc(N)c(CC)c1]

In some embodiments, Ring A is:

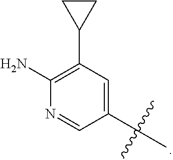 [SMILES: *c1cnc(N)c(C2CC2)c1]

In some embodiments, Ring A is:

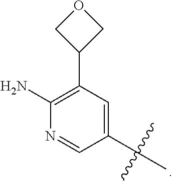 [SMILES: *c1cnc(N)c(C2COC2)c1]

In some embodiments, Ring A is:

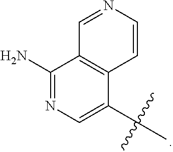 [SMILES: *c1cnc(N)c2cnccc12]

In some embodiments, Ring A is:

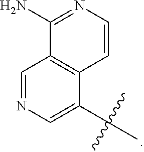 [SMILES: *c1cncc2c(N)nccc12]

In some embodiments, Ring A is:

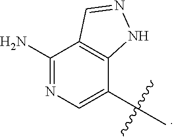 [SMILES: *c1cnc(N)c2cn[nH]c12]

In some embodiments, Ring A is:

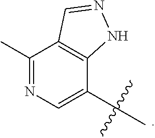 [SMILES: *c1cnc(C)c2cn[nH]c12]

In some embodiments, Ring A is:

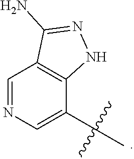 [SMILES: *c1cncc2c(N)n[nH]c12]

In some embodiments, Ring A is:

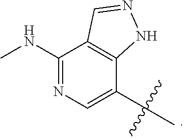 [SMILES: *c1cnc(NC)c2cn[nH]c12]

In some embodiments, Ring A is

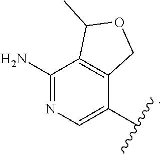 [SMILES: *c1cnc(N)c2c1COC2C]

In some embodiments, Ring A is

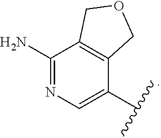 [SMILES: *c1cnc(N)c2c1COC2]

In some embodiments, Ring A is

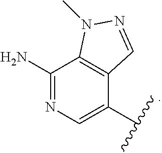 [SMILES: *c1cnc(N)c2c1cnn2C]

In some embodiments, Ring A is:

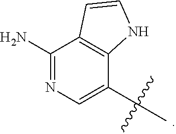 [SMILES: *c1cnc(N)c2cc[nH]c12]

In some embodiments, Ring A is:

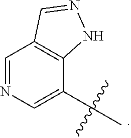 [SMILES: *c1cncc2cn[nH]c12]

In some embodiments, Ring A is:

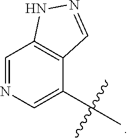 [SMILES: *c1cncc2[nH]ncc12]

In some embodiments, Ring A is:

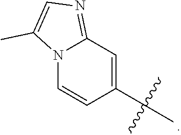 [SMILES: *c1ccn2c(C)cnc2c1]

In some embodiments, Ring A is:

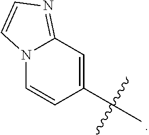 [SMILES: *c1ccn2ccnc2c1]

In some embodiments the compounds of Formula (I) are of Formula (I_1):

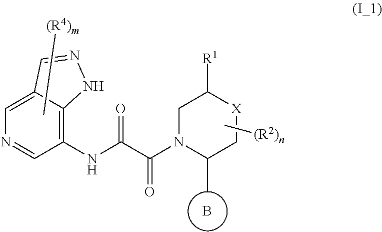

wherein X, Ring B, R¹, R², R⁴, m and n are as defined herein.

In some embodiments, the compounds are of Formula (I_1a):

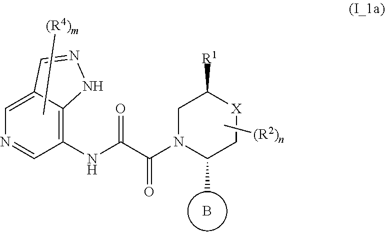

wherein X, Ring B, R¹, R², R⁴, m and n are as defined herein.

In some embodiments the compounds of Formula (I) are of Formula (I_2):

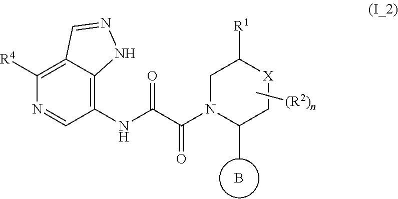

wherein X, Ring B, R¹, R², R⁴ and n are as defined herein.

In some embodiments, the compounds are of Formula (I_2a):

wherein X, Ring B, R¹, R², R⁴ and n are as defined herein.

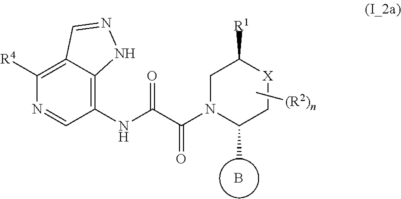

In some embodiments the compounds of Formula (I) are of Formula (I_3):

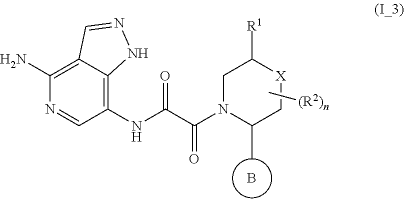

wherein X, Ring B, R¹, R² and n are as defined herein.

In some embodiments, the compounds are of Formula (I_3a):

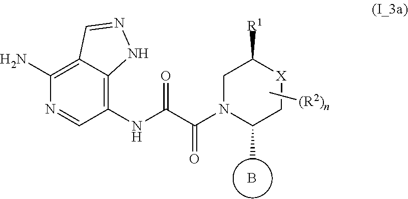

wherein X, Ring B, R¹, R² and n are as defined herein.

In some embodiments the compounds of Formula (I) are of Formula (I_4):

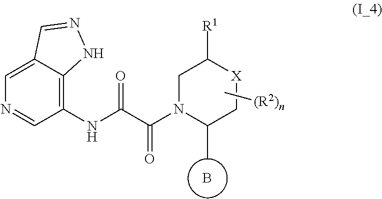

wherein X, Ring B, R¹, R² and n are as defined herein.

In some embodiments, the compounds are of Formula (I_4a):

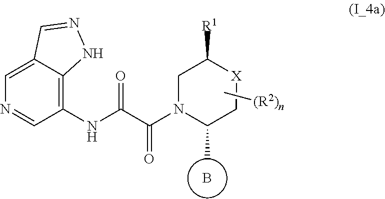

wherein X, Ring B, R¹, R² and n are as defined herein.

In some embodiments the compounds of Formula (I) are of Formula (I_5):

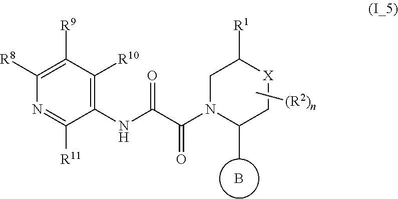

wherein X, Ring B, R¹, R², R⁸, R R¹, R¹¹ and n are as defined herein.

In some embodiments, the compounds are of Formula (I_5a):

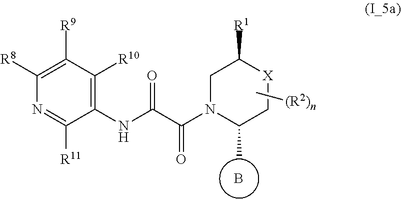

wherein X, Ring B, R¹, R², R⁸, R⁹, R¹⁰, R¹¹ and n are as defined herein.

In some embodiments the compounds of Formula (I) are of Formula (I_6):

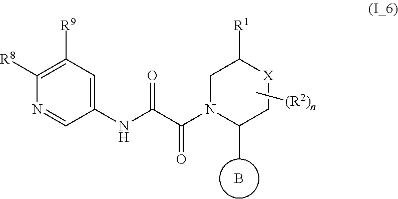

wherein X, Ring B, R¹, R², R⁸, R⁹ and n are as defined herein.

In some embodiments, the compounds are of Formula (I_6a):

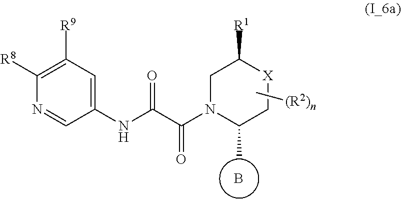

wherein X, Ring B, R¹, R², R⁸, R⁹ and n are as defined herein.

In some embodiments the compounds of Formula (I) are of Formula (I_7):

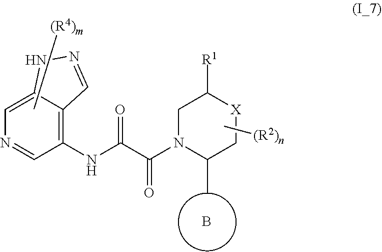

wherein X, Ring B, R¹, R², R⁴, m and n are as defined herein.

In some embodiments, the compounds are of Formula (I_7a):

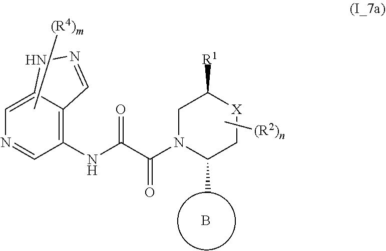

wherein X, Ring B, R¹, R², R⁴, m and n are as defined herein.

In some embodiments the compounds of Formula (I) are of Formula (I_8):

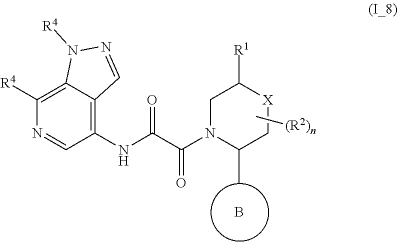

wherein X, Ring B, R¹, R², R⁴ and n are as defined herein.

In some embodiments, the compounds are of Formula (I_8a):

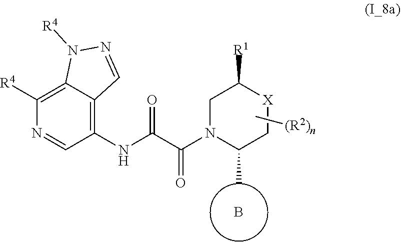

wherein X, Ring B, R¹, R², R⁴ and n are as defined herein.

In some embodiments the compounds of Formula (I) are of Formula (I_9):

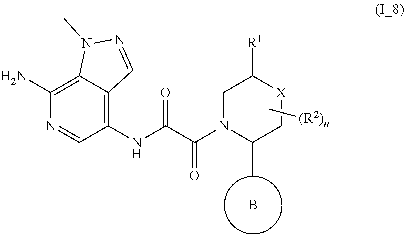

wherein X, Ring B, R¹, R² and n are as defined herein.

In some embodiments, the compounds are of Formula (I_9a):

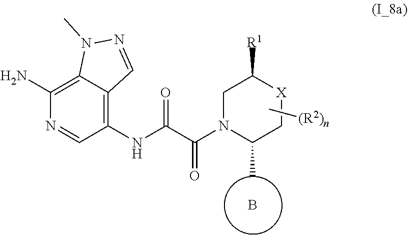

wherein X, Ring B, R¹, R² and n are as defined herein.

In some embodiments the compounds of Formula (I) are of Formula (II_1):

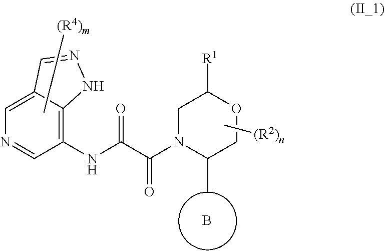

wherein Ring B, R¹, R², R⁴, m and n are as defined herein.

In some embodiments, the compounds are of Formula (I_1a):

wherein Ring B, R¹, R², R⁴, m and n are as defined herein.

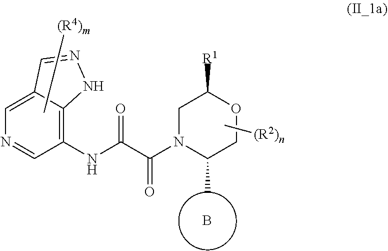

In some embodiments the compounds of Formula (I) are of Formula (II_2):

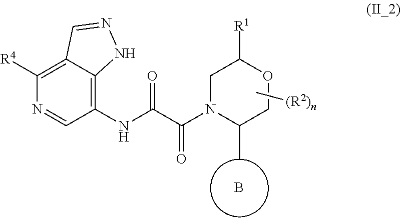

wherein Ring B, R¹, R², R⁴ and n are as defined herein.

In some embodiments, the compounds are of Formula (I_1_2a):

wherein Ring B, R¹, R², R⁴ and n are as defined herein.

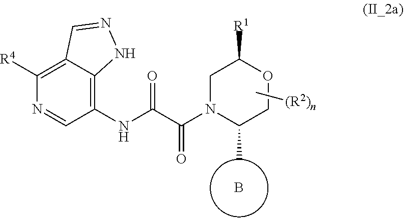

In some embodiments the compounds of Formula (I) are of Formula (II_3):

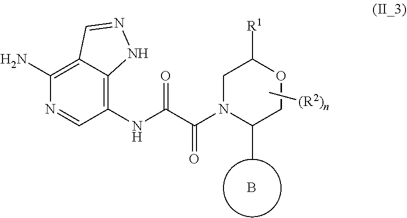

wherein Ring B, R¹, R² and n are as defined herein.

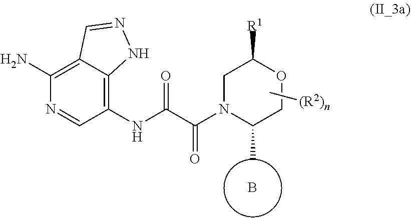

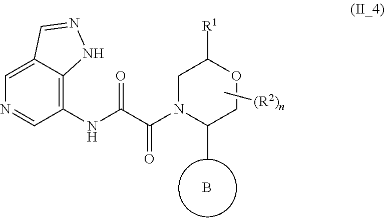

In some embodiments, the compounds are of Formula (I_1_4a):

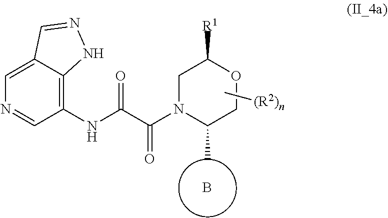

wherein Ring B, R¹, R² and n are as defined herein.

In some embodiments the compounds of Formula (I) are of Formula (II_5):

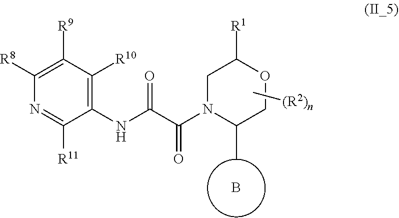

wherein Ring B, R¹, R², R⁸, R⁹, R¹⁰, R¹¹ and n
are as defined herein.

In some embodiments, the compounds are of Formula (I_15a):

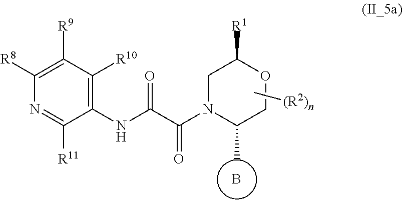

wherein Ring B, R¹, R², R⁸, R⁹, R¹⁰, R¹¹ and n are as defined herein.

In some embodiments the compounds of Formula (I) are of Formula (II_6):

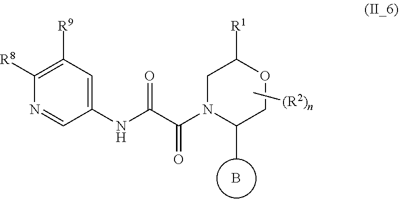

wherein Ring B, R¹, R², R⁸, R⁹ and n are as defined herein.

In some embodiments, the compounds are of Formula (I_16a):

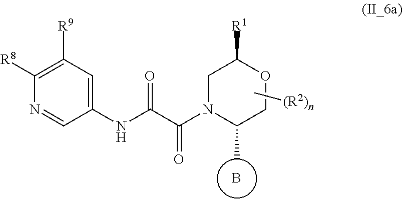

wherein Ring B, R¹, R², R⁸, R⁹ and n are as defined herein.

In some embodiments the compounds of Formula (I) are of Formula (II_7):

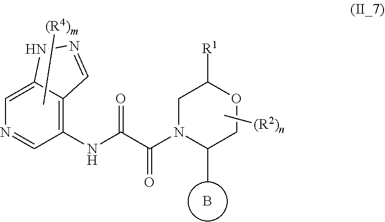

wherein Ring B, R¹, R², R⁴, m and n are as defined herein.

In some embodiments, the compounds are of Formula (I_17a):

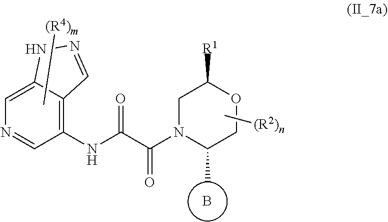

wherein Ring B, R¹, R², R⁴, m and n are as defined herein.

In some embodiments the compounds of Formula (I) are of Formula (II_8):

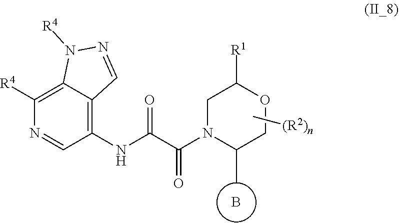

wherein Ring B, R¹, R², R⁴ and n are as defined herein.

In some embodiments, the compounds are of Formula (I_18a):

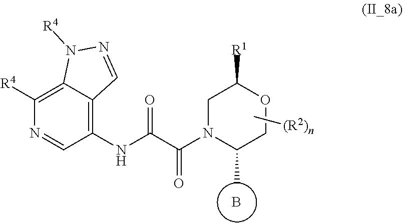

wherein Ring B, R¹, R², R⁴ and n are as defined herein.

In some embodiments the compounds of Formula (I) are of Formula (II_9):

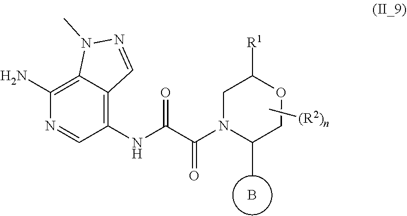

wherein Ring B, R¹, R² and n are as defined herein.

In some embodiments, the compounds are of Formula (I_19a):

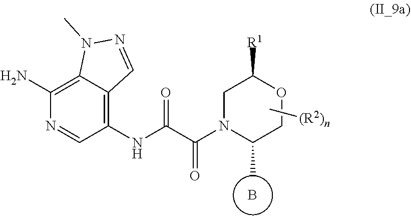

wherein Ring B, Rand R² and n are as defined herein.

In some embodiments the compounds of Formula (I) are of Formula (III_1):

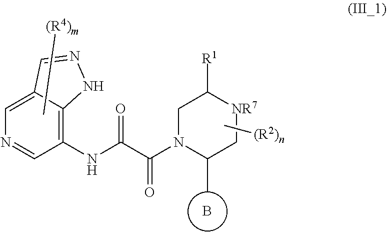

wherein R⁷, Ring B, R¹, R², R⁴, m and n are as defined herein.

In some embodiments, the compounds are of Formula (III_1a):

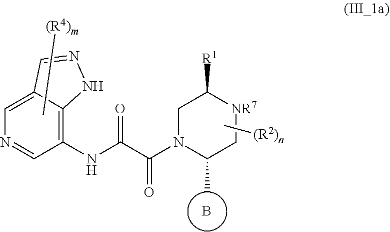

wherein R⁷, Ring B, R¹, R², R⁴, m and n are as defined herein.

In some embodiments the compounds of Formula (I) are of Formula (III_2):

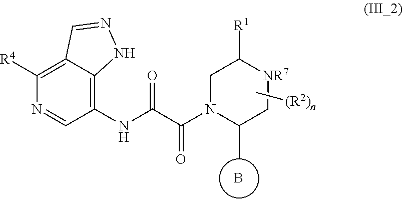

wherein R⁷, Ring B, R¹, R², R⁴ and n are as defined herein.

In some embodiments, the compounds are of Formula (III_2a):

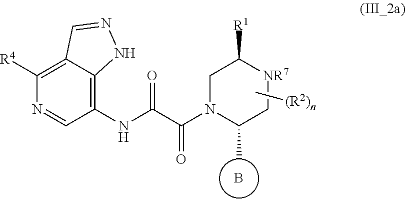

wherein R⁷, Ring B, R¹, R², R⁴ and n are as defined herein.

In some embodiments the compounds of Formula (I) are of Formula (III_3):

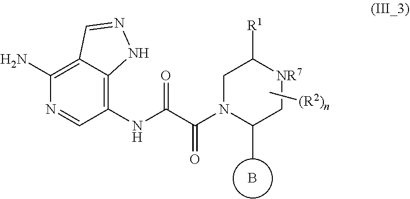

wherein R⁷, Ring B, R¹, R² and n are as defined herein.

In some embodiments, the compounds are of Formula (III_3a):

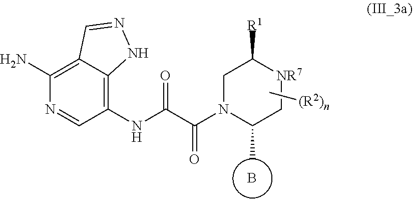

wherein R⁷, Ring B, R¹, R² and n are as defined herein.

In some embodiments the compounds of Formula (I) are of Formula (III_4):

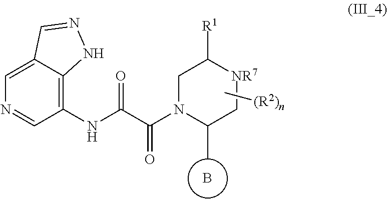

wherein R⁷, Ring B, R¹, R² and n are as defined herein.

In some embodiments, the compounds are of Formula (III_4a):

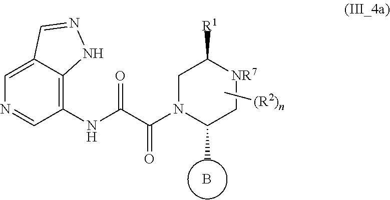

wherein R⁷, Ring B, R¹, R² and n are as defined herein.

In some embodiments the compounds of Formula (I) are of Formula (III_5):

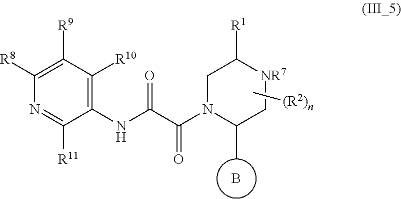

wherein R⁷, Ring B, R¹, R², R⁸, R R¹, R¹¹ and n are as defined herein.

In some embodiments, the compounds are of Formula (III_5a):

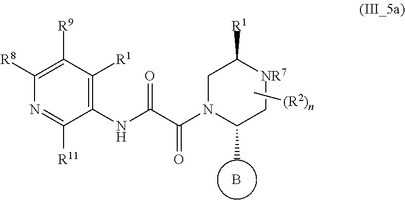

wherein R⁷, Ring B, R¹, R², R⁸, R⁹, R¹⁰, R¹¹ and n are as defined herein.

In some embodiments the compounds of Formula (I) are of Formula (III_6):

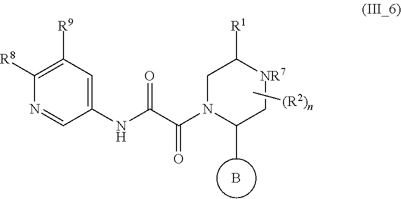

wherein R⁷, Ring B, R¹, R², R⁸, R⁹ and n are as defined herein.

In some embodiments, the compounds are of Formula (III_6a):

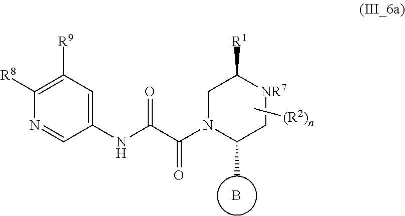

wherein R⁷, Ring B, R¹, R², R⁸, R⁹ and n are as defined herein.

In some embodiments the compounds of Formula (I) are of Formula (III_7):

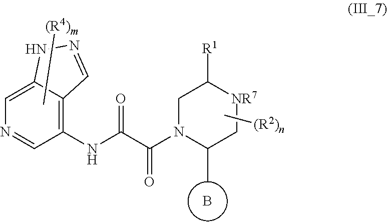

wherein R⁷, Ring B, R¹, R², R⁴, m and n are as defined herein.

In some embodiments, the compounds are of Formula (III_7a):

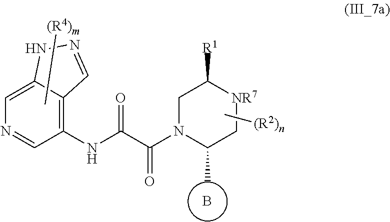

wherein R⁷, Ring B, R¹, R², R⁴, m and n are as defined herein.

In some embodiments the compounds of Formula (I) are of Formula (III_8):

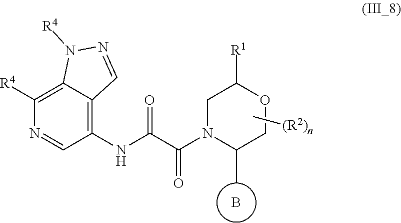

wherein R⁷, Ring B, R¹, R², R⁴ and n are as defined herein.

In some embodiments, the compounds are of Formula (III_8a):

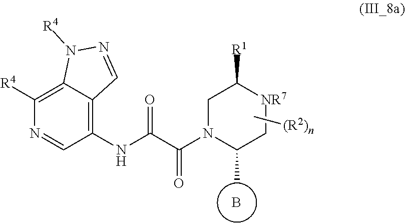

wherein R⁷, Ring B, R¹, R², R⁴ and n are as defined herein.

In some embodiments the compounds of Formula (I) are of Formula (III_9):

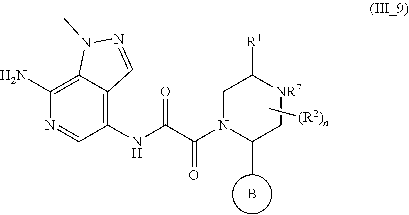

wherein R⁷, Ring B, R¹, R² and n are as defined herein.

In some embodiments, the compounds are of Formula (III_9a):

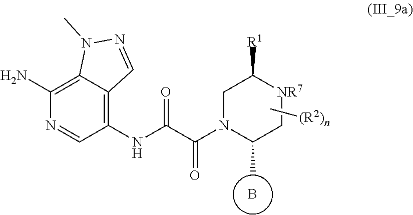

wherein R⁷, Ring B, R¹, R² and n are as defined herein.

As generally described herein, each R⁴ is independently selected from the group consisting of -D, halo, ═O, —CN, —C₁-C₆ alkyl, —C₁-C₆ heteroalkyl, —C₁-C₆ haloalkyl, —C₃—C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(0R^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂, wherein R⁴ is as defined herein.

In some embodiments, each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂, wherein R^{a4} is as defined herein.

In some embodiments, each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a4}, —N(R^{a4})₂, —C(═O)R^{a4}, —C(=)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂, wherein R^{a4} is as defined herein.

In some embodiments, each R⁴ is independently selected from the group consisting of -D, ═O, —C₁-C₆ alkyl, —C₁-C₆ haloalkyl, —C₃-C₉ cycloalkyl, —OR⁴ and —N(R^{a4})₂.

In some embodiments, each R⁴ is independently selected from the group consisting of -D, ═O, —C₁-C₆ alkyl, —C₁-C₆ haloalkyl, —C₃-C₉ cycloalkyl, —OR^{a4} and —N(R^{a4})₂.

In some embodiments, each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂, wherein R^{a4} is as defined herein.

In certain embodiments, each R⁴ is independently selected from the group consisting of ═O, —C₁-C₆ alkyl, —C₁-C₆ haloalkyl, —C₃-C₉ cycloalkyl, 3-10 membered heterocyclyl, —C(═O)N(R^{a4})₂, —OR^{a4} and —N(R^{a4})₂, wherein R^{a4} is as defined herein.

In some embodiments, each R⁴ is independently selected from the group consisting of -D, ═O, —C₁-C₆ alkyl and —N(R^{a4})₂.

In certain embodiments, each R⁴ is independently selected from the group consisting of ═O, —C₁-C₆ alkyl, —C₁-C₆ haloalkyl, —C₃-C₉ cycloalkyl, —OR^{a4} and —N(R^{a4})₂, wherein R^{a4} is as defined herein.

In some embodiments, each R⁴ is independently selected from the group consisting of ═O, —C₁-C₆ alkyl, 3-10 membered heterocyclyl, —OR^{a4}, —C(═O)N(R^{a4})₂ and —N(R^{a4})₂, wherein R^{a4} is as defined herein. In some embodiments, each R^{a4} is independently selected from the group consisting of H and -Me.

In some embodiments, each R⁴ is independently selected from the group consisting of ═O, —C₁-C₆ alkyl and —N(R^{a4})₂, wherein R^{a4} is as defined herein.

In some embodiments, each R⁴ is independently selected from the group consisting of ═O, -Me, -Et, —ⁱPr, -Bu, cyclopropyl, oxetanyl (e.g., oxetan-3-yl), —OCH₃, —C(═O)NH₂, —NH₂, —NHCH₃ and —NH(CH₃)₂.

In some embodiments, each R⁴ is independently selected from the group consisting of ═O, -Me, -Et, —ⁱPr, -^{t}Bu, —NH₂, —NHCH₃ and —NH(CH₃)₂.

In some embodiments, R⁴ is selected from the group consisting of cyclopropyl, oxetanyl (e.g., oxetan-3-yl), —C(═O)NH₂, —NHCH₃, —NH₂, —OCH₃, -Et or -Me.

In some embodiments, R⁴ is selected from the group consisting of —NHCH₃, —NH₂ or -Me.

In some embodiments, R⁴ is selected from the group consisting of —NH₂ or -Me.

In some embodiments, R⁴ is ═O.

In some embodiments, R⁴ is D.

In certain embodiments, R⁴ is halo (e.g., fluoro, chloro, bromo, iodo). In some embodiments, R⁴ is —Cl. In some embodiments, R⁴ is —F. In some embodiments, R⁴ is —Br. In some embodiments, R⁴ is —I.

In some embodiments, R⁴ is —CN.

In certain embodiments, R⁴ is —C₁-C₆ alkyl. In some embodiments, R⁴ is -Me. In some embodiments, R⁴ is -Et. In some embodiments R⁴ is —Pr or -iPr.

In some embodiments, R⁴ is —C₁-C₆ heteroalkyl. In some embodiments, R⁴ is methoxymethyl (—CH₂OCH₃). In some embodiments, R⁴ is hydroxymethyl (—CH₂OH). In some embodiments, R⁴ is aminomethyl (e.g., —CH₂NH₂, —CH₂NHCH₃, —CH₂N(CH₃)₂.

In some embodiments, R⁴ is —C₁-C₆ haloalkyl. In some embodiments, R⁴ is trifluoromethyl (—CF₃). In other embodiments, R⁴ is difluoromethyl (—CHF₂).

In some embodiments, R⁴ is C₃-C₉ cycloalkyl (e.g., cyclopropyl, cyclobutyl, cyclopentyl, cyclohexyl). In some embodiments, R⁴ is cyclopropyl. In some embodiments R⁴ is cyclobutyl. In some embodiments, R⁴ is cyclopentyl. In some embodiments, R⁴ is cyclohexyl.

In some embodiments, R⁴ is 3-10 membered heterocyclyl. In some embodiments, R⁴ is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁴ is oxetanyl (e.g., oxetan-3-yl). In some embodiments, R⁴ is tetrahydropyranyl. In some embodiments, R⁴ is tetrahydrofuranyl. In some embodiments, R⁴ is azetidinyl. In some embodiments, R⁴ is pyrrolidinyl. In some embodiments, R⁴ is piperidinyl. In some embodiments, R⁴ is piperazinyl. In some embodiments, R⁴ is morpholinyl. In some embodiments, R⁴ is azepanyl.

In some embodiments R⁴ is cycloalkylalkyl (e.g., cyclopropylmethyl, cyclobutylmethyl, cyclopentylmethyl, cyclohexylmethyl, cycloheptylmethyl).

In some embodiments, R⁴ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁴ is arylalkyl. In some embodiments, R⁴ is benzyl.

In some embodiments, R⁴ is heteroarylalkyl (e.g., pyridinylmethyl, thiazolylmethyl, triazolylmethyl, pyrazolylmethyl).

In some embodiments, R⁴ is —OR^{a4} wherein R^{a4} is as defined in any of the embodiments described herein (e.g., hydroxy (—OH), methoxy, ethoxy, propoxy, isopropoxy, cyclopropyloxy, cyclobutyloxy). In some embodiments, R⁴ is hydroxy. In some embodiments, R⁴ is methoxy. In some embodiments, R⁴ is ethoxy. In some embodiments, R⁴ is propoxy. In some embodiments, R⁴ is isopropoxy. In some embodiments, R⁴ is —C₁-C₆ haloalkoxy. In some embodiments, R⁴ is trifluoromethoxy (—OCF₃), In other embodiments, R⁴ is difluoromethoxy (—OCHF₂).

In some embodiments, R⁴ is —N(R^{a4})₂ wherein R^{a4} is as defined in any of the embodiments described herein (e.g., —NH₂, —NHR^{a4}, —N(CH₃)R^{a4}). In some embodiments, R⁴ is —NH₂. In some embodiments, R⁴ is —NHR^{a4} (e.g., —NHCH₃, —NHCH₂CH₃, —NHPr, —NHⁱPr, -NHcyclopropyl, -NHcyclobutyl). In some embodiments, R⁴ is —N(CH₃)R^{a4} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R⁴ is —C(═O)R^{a4} or —C(═O)OR^{a4} wherein R^{a4} is as defined in any of the embodiments described herein. In some embodiments, R⁴ is —C(═O)R^{a4} wherein R^{a4} is as defined in any of the embodiments described herein. In some embodiments, R⁴ is —C(═O)alkyl. In some embodiments, R⁴ is —C(═O)CH₃, —C(═O)cyclopropyl, —C(═O)cyclobutyl, —C(═O)^{t}Bu, —C(═O)ⁱPr, —C(═O)CH₂CH₂CH₃ or —C(═O)OCH₃. In some embodiments, R⁴ is acetyl (—C(═O)CH₃). In some embodiments, R⁴ is —C(═O)OR^{a4}. In some embodiments, R⁴ is —COOH. In some embodiments, R⁴ is COOCH₃.

In some embodiments, R⁴ is —NR^{a4}C(═O)R^{a4} wherein R^{a4} is as defined in any of the embodiments described herein. In certain embodiments, R⁴ is —NHC(═O)R^{a4} (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)^{t}Bu, —NHC(═O)Cyclopropyl, —NHC(═O)Cyclobutyl). In some embodiments, R⁴ is —N(CH₃)C(═O)R^{a4} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁴ is —NR^{a4}C(═O)OR^{a4} wherein R^{a4} is as defined in any of the embodiments described herein. In certain embodiments, R⁴ is —NHC(═O)OR^{a4} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R⁴ is —N(CH₃)C(═O)OR^{a4} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁴ is —C(═O)N(R^{a4})₂ wherein R^{a4} is as defined in any of the embodiments described herein (e.g., —C(═O)NH₂, —C(═O)NHR^{a4}, —C(═O)N(CH₃)R^{a4}). In some embodiments, R⁴ is —C(═O)NH₂. In certain embodiments, R⁴ is —C(═O)NHR^{a4} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁴ is —C(═O)N(CH₃)R^{a4} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁴ is —C(═O)N(OR^{a4})(R^{a4}). In certain embodiments, R⁴ is —C(═O)NH(OR^{a4}) (e.g., —C(═O)NHOH, —C(═O)NHOCH3). In some embodiments, R⁴ is —C(═O)NHOH.

In some embodiments, R⁴ is —OC(═O)N(R^{a4})₂ wherein R^{a4} is as defined in any of the embodiments described herein. In certain embodiments, R⁴ is —OC(═O)NHR^{a4} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁴ is —OC(═O)N(CH₃)R^{a4} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁴ is —S(═O)R^{a4} wherein R^{a4} is as defined in any of the embodiments described herein. In certain embodiments, R⁴ is —S(═O)alkyl (e.g., —S(═O)CH₃, —S(═O)CH₂CH₃, —S(═O)CH₂CH₂CH₃, —S(═O)ⁱPr). In certain embodiments, R⁴ is —S(═O)cycloalkyl (e.g., —S(═O)cyclopropyl, —S(═O)cyclobutyl, —S(═O)cyclopentyl, —S(═O)cyclohexyl).

In some embodiments, R⁴ is —S(═O)₂R^{a4} wherein R^{a4} is as defined in any of the embodiments described herein. In certain embodiments, R⁴ is —S(═O)₂alkyl (e.g., —S(═O)₂CH₃, —S(═O)₂CH₂CH₃, —S(═O)₂Pr, —S(═O)₂′Pr). In certain embodiments, R⁴ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁴ is S(═O)₂aryl (e.g., —S(═O)₂phenyl).

In some embodiments, R⁴ is —SR^{a4} wherein R^{a4} is as defined in any of the embodiments described herein. In certain embodiments, R⁴ is -Salkyl (e.g., —SCH₃, —SCH₂CH₃, —SPr, —SⁱPr). In certain embodiments, R⁴ is -Scycloalkyl (e.g., -Scyclopropyl, -Scyclobutyl, -Scyclopentyl, -Scyclohexyl). In certain embodiments, R⁴ is -Saryl (e.g., -Sphenyl).

In some embodiments, R⁴ is —S(═O)(═NR^{a4})R^{a4} wherein R^{a4} is as defined in any of the embodiments described herein. In certain embodiments, R⁴ is —S(═O)(═NH)R^{a4} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁴ is —S(═O)(═NCH₃)R^{a4} (e.g., —S(═O)(═NCH₃)CH, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁴ is —NR^{a4}S(═O)₂R^{a4} wherein R^{a4} is as defined in any of the embodiments described herein. In certain embodiments, R⁴ is —NHS(═O)₂alkyl (e.g., —NHS(═O)₂CH₃, —NHS(═O)₂CH₂CH₃, —NHS(═O)₂Pr, —NHS(═O)₂′Pr). In certain embodiments, R⁴ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁴ is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂′Pr). In certain embodiments, R⁴ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁴ is —S(═O)₂N(R^{a4})₂ wherein R^{a4} is as defined in any of the embodiments described herein. (e.g., —S(═O)₂NH₂, —S(═O)₂NHR^{a4}, —S(═O)₂N(CH₃)R^{a4}). In some embodiments, R⁴ is —S(═O)₂NH₂. In some embodiments, R⁴ is —S(═O)₂NHR^{a4} (e.g., —S(═O)₂NHCH₃, —S(═O)₂NHCH₂CH₃, —S(═O)₂NHPr, —S(═O)₂NH′Pr, —S(═O)₂NHcyclopropyl, —S(═O)₂NHcyclobutyl). In some embodiments, R⁴ is —S(═O)₂N(CH₃)R^{a4} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R⁸ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, -ORa⁸, —N(R^{a8})₂, —C(═O)Ra′, —C(═O)ORa⁸, -NRa⁸C(═O)Ra⁸, —NR^{a8}C(═O)OR^{a8}, —C(═O)N(R^{a8})₂, —OC(═O)N(R^{a8})₂, —S(═O)Ra⁸, —S(═O)₂Ra⁸, -SRa⁸, —S(═O)(═NR^{a8})R^{a8}, —NR^{a8}S(═O)₂R^{as} and —S(═O)₂N(R^{a8})₂, wherein R^{a8} is as defined herein.

In some embodiments, R⁸ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a8}, —N(R^{ag})₂, —C(═O)R^{ag}, —C(═O)OR^{a8}, —NR^{a8}C(═O)R^{a8}, —NR^{a8}C(═O)OR^{a8}, —C(═O)N(R^{a8})₂ and —OC(═O)N(R^{a8})₂ wherein R^{a8} is as defined herein.

In certain embodiments, R⁸ is selected from the group consisting of H, halo, —CN, —C₁-C₆ alkyl, —C₁-C₆ haloalkyl, —OR^{as} and —N(R^{a8})₂ wherein R^{a8} is as defined herein.

In some embodiments, R⁸ is selected from the group consisting of H, —C₁-C₆ alkyl, —C₁-C₆ haloalkyl, —OR^{as} and —N(R^{a8})₂ wherein R^{a8} is as defined herein.

In some embodiments, R⁸ is selected from the group consisting of OR^{as} and —N(R^{a8})₂ wherein R^{a8} is as defined herein.

In some embodiments, each R^{a8} is independently selected from the group consisting of H, —C₁-C₆ alkyl (e.g., -Me, -Et, —Pr, —ⁱPr, -nBu, -^{t}Bu, -sec-Bu, -iso-Bu) and —C₁-C₆ haloalkyl (e.g., —CHF₂, —CF₃).

In some embodiments, R⁸ is selected from the group consisting of H, —C₁-C₆ alkyl (e.g., -Me, -Et, —Pr, -iPr, -nBu, -tBu, -sec-Bu, -iso-Bu), —C₁-C₆ alkyl (e.g., —CF₃, —CHF₂), —OH, —O—(C₁-C₆ alkyl) (e.g., —OCH₃, -OEt), —O—(C₁-C₆ haloalkyl) (e.g., —OCF₃, —OCHF₂), —NH₂, —NH—(C₁-C₆ alkyl) (e.g., —NHCH₃) and —N—(C₁-C₆ alkyl)₂ (e.g, —N(CH₃)₂).

In certain embodiments, R⁸ is selected from the group consisting of H, -Me, -Et, —CHF₂, —OCH₃, -OEt, —OCHF₂, —OCF₃, —OH and —NH₂. In some embodiments, R⁸ is selected from the group consisting of H, -Et, —OCH₃, -OEt, —OCHF₂, —OCF₃ and —OH.

In certain embodiments, R⁸ is selected from the group consisting of H, -Me, —CHF₂, —OCH₃ and —NH₂

In other embodiments, R⁸ is selected from the group consisting of H, -Me, —CHF₂ and —NH₂. In some embodiments, R⁸ is selected from the group consisting of -Me and —NH₂.

In some embodiments, R⁸ is selected from the group consisting of —NH₂ and —OCH₃.

In some embodiments, R⁸ is H. In some embodiments R⁸ is -D.

In certain embodiments, R⁸ is halo (e.g., fluoro, chloro, bromo, iodo). In some embodiments, R⁸ is —Cl. In some embodiments, R⁸ is —F. In some embodiments, R⁸ is —Br. In some embodiments, R⁸ is —I.

In some embodiments, R⁸ is —CN.

In certain embodiments, R⁸ is —C₁-C₆ alkyl. In some embodiments, R⁸ is -Me. In some embodiments, R⁸ is -Et. In some embodimentsR⁸ is —Pr or -iPr.

In some embodiments, R⁸ is —C₁-C₆ heteroalkyl. In some embodiments, R⁸ is methoxymethyl (—CH₂OCH₃). In some embodiments, R⁸ is hydroxymethyl (—CH₂OH). In some embodiments, R⁸ is aminomethyl (e.g., —CH₂NH₂, —CH₂NHCH₃, —CH₂N(CH₃)₂. In some embodiments, R⁸ is —C₁-C₆ haloalkyl. In some embodiments, R⁸ is trifluoromethyl (—CF₃). In other embodiments, R⁸ is difluoromethyl (—CHF₂).

In some embodiments, R⁸ is —C₃-C₉ cycloalkyl (e.g., cyclopropyl, cyclobutyl, cyclopentyl, cyclohexyl). In some embodiments, R is cyclopropyl. In some embodiments R⁸ is cyclobutyl. In some embodiments, R⁸ is cyclopentyl. In some embodiments, R⁸ is cyclohexyl.

In some embodiments, R⁸ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R is oxetanyl. In some embodiments, R⁸ is tetrahydropyranyl. In some embodiments, R⁸ is tetrahydrofuranyl. In some embodiments, R⁸ is azetidinyl. In some embodiments, R⁸ is pyrrolidinyl. In some embodiments, R⁸ is piperidinyl. In some embodiments, R⁸ is piperazinyl. In some embodiments, R⁸ is morpholinyl. In some embodiments, R⁸ is azepanyl.

In some embodiments R⁸ is cycloalkylalkyl (e.g., cyclopropylmethyl, cyclobutylmethyl, cyclopentylmethyl, cyclohexylmethyl, cycloheptylmethyl). In some embodiments, R⁸ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁸ is arylalkyl. In some embodiments, R⁸ is benzyl. In some embodiments, R⁸ is heteroarylalkyl (e.g., pyridinylmethyl, thiazolylmethyl, triazolylmethyl, pyrazolylmethyl).

In some embodiments, R⁸ is —OR^{as} wherein R^{a8} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R⁸ is hydroxy. In some embodiments, R⁸ is methoxy. In some embodiments, R⁸ is ethoxy. In some embodiments, R⁸ is propoxy. In some embodiments, R⁸ is isopropoxy. In some embodiments R⁸ is difluoromethoxy. (—OCHF₂). In some embodiments, R⁸ is trifluoromethoxy (—OCF₃).

In some embodiments, R⁸ is —N(R^{a8})₂ wherein R^{a8} is as defined in any of the embodiments described herein (e.g., —NH₂, —NHR^{a11} s, —N(CH₃)R^{a8}). In some embodiments, R⁸ is —NH₂. In some embodiments, R⁸ is —NHR^{a8} (e.g., —NHCH₃, -NHEt, —NHPr, —NHⁱPr, -NHcyclopropyl, -NHcyclobutyl). In some embodiments, R⁸ is —N(CH₃)R^{a8} (e.g., —N(CH₃)₂, —N(CH₃)Et, —N(CH₃)Pr, —N(CH₃)ⁱPr, —N(CH₃)cyclopropyl, —N(CH₃)cyclobutyl).

In some embodiments, R⁸ is —C(═O)R^{a8} or —C(═O)OR^{a8}. In some embodiments, R⁸ is —C(═O)R^{a8} wherein R^{a8} is as defined in any of the embodiments described herein. In some embodiments, R⁸ is —C(═O)alkyl. In some embodiments, R⁸ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R⁸ is acetyl (—C(═O)Me). In some embodiments, R⁸ is —C(═O)ORa. In some embodiments, R⁸ is —COOH. In some embodiments, R⁸ is COOCH₃.

In some embodiments, R⁸ is —NR^{a8}C(═O)R^{a8} wherein R^{a8} is as defined in any of the embodiments described herein. In certain embodiments, R⁸ is —NHC(═O)R^{a8} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁸ is —N(CH₃)C(═O)R^{a8} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁸ is —NR^{a8}C(═O)OR^{as} wherein R^{a8} is as defined in any of the embodiments described herein. In certain embodiments, R⁸ is —NHC(═O)OR^{as}(eg —NHC(═O)OCH₃, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, 5 R⁸ is —N(CH₃)C(═O)OR^{as}(e.g., —N(CH₃)C(═O)OCH₃, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁸ is —C(═O)N(R^{a8})₂ wherein R^{a8} is as defined in any of the embodiments described herein (e.g., —C(═O)NH₂, —C(═O)NHR^{a11} s, —C(═O)N(CH₃)R^{a8}). In some embodiments, R⁸ is —C(═O)NH₂. In certain embodiments, R⁸ is —C(═O)NHR^{as}(e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁸ is —C(═O)N(CH₃)R^{a8} (e.g., —C(═O)N(CH₃)₂, —C(═O)N(CH₃)Et, —C(═O)N(CH₃)Pr, —C(═O)N(CH₃)ⁱPr, —C(═O)N(CH₃)Bu, —C(═O)N(CH₃)Bu, —C(═O)N(CH₃)Cyclopropyl, —C(═O)N(CH₃)Cyclobutyl).

In some embodiments, R⁸ is —OC(═O)N(R^{a8})₂ wherein R^{a8} is as defined in any of the embodiments described herein. In certain embodiments, R⁸ is —OC(═O)NHR^{a}(e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁸ is —OC(═O)N(CH₃)R^{a8} (e.g., —OC(═O)N(CH₃)₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)Bu, —OC(═O)N(CH₃)Cyclopropyl, —OC(═O)N(CH₃)Cyclobutyl).

In some embodiments, R⁸ is —S(═O)R^{as} wherein R^{a8} is as defined in any of the embodiments described herein. In certain embodiments, R⁸ is —S(═O)alkyl (e.g., —S(═O)Me, —S(═O)Et, —S(═O)Pr, —S(═O)ⁱPr). In certain embodiments, R⁸ is —S(═O)cycloalkyl (e.g., —S(═O)cyclopropyl, —S(═O)cyclobutyl, —S(═O)cyclopentyl, —S(═O)cyclohexyl).

In some embodiments, R⁸ is —S(═O)₂R^{as} wherein R^{a8} is as defined in any of the embodiments described herein. In certain embodiments, R⁸ is —S(═O)₂alkyl (e.g., —S(═O)₂Me, —S(═O)₂Et, —S(═O)₂Pr, —S(═O)₂′Pr). In certain embodiments, R⁸ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁸ is S(═O)₂aryl (e.g., —S(═O)₂phenyl).

In some embodiments, R⁸ is —SR^{as} wherein R^{a8} is as defined in any of the embodiments described herein. In certain embodiments, R⁸ is -Salkyl (e.g., —SMe, -SEt, —SPr, —SⁱPr). In certain embodiments, R⁸ is -Scycloalkyl (e.g., -Scyclopropyl, -Scyclobutyl, -Scyclopentyl, -Scyclohexyl). In certain embodiments, R⁸ is -Saryl (e.g., -Sphenyl).

In some embodiments, R⁸ is —S(═O)(═NR^{a8})R^{a8} wherein R^{a8} is as defined in any of the embodiments described herein. In certain embodiments, R⁸ is —S(═O)(═NH)R^{a8} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁸ is —S(═O)(═NCH₃)R^{a8} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁸ is —NR^{a8}S(═O)₂R^{as} wherein R^{a8} is as defined in any of the embodiments described herein. In certain embodiments, R⁸ is —NHS(═O)₂alkyl (e.g., —NHS(═O)₂Me, —NHS(═O)₂Et, —NHS(═O)₂Pr, —NHS(═O)₂′Pr). In certain embodiments, R⁸ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁸ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R⁸ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁸ is —S(═O)₂N(R^{a8})₂ wherein R^{a8} is as defined in any of the embodiments described herein. (e.g., —S(═O)₂NH₂, —S(═O)₂NHR^{a11} s, —S(═O)₂N(CH₃)R^{a8}). In some embodiments, R⁸ is —S(═O)₂NH₂. In some embodiments, R⁸ is —S(═O)₂NHR^{a}(e.g., —S(═O)₂NHCH₃, —S(═O)₂NHEt, —S(═O)₂NHPr, —S(═O)₂NH′Pr, —S(═O)₂NHcyclopropyl, —S(═O)₂NHcyclobutyl). In some embodiments, R⁸ is —S(═O)₂N(CH₃)R^{a8} (e.g., —S(═O)₂N(CH₃)₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R⁹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a9}, —N(R^{a9})₂, —C(═O)R^{a9}, —C(═O)OR^{a9}, —NR^{a9}C(═O)R^{a9}, —NR^{a9}C(═O)OR^{a9}, —C(═O)N(R^{a9})₂, —OC(═O)N(R^{a9})₂, —S(═O)R^{a9}, —S(═O)₂R^{a9}, —SR^{a9}, —S(═O)(═NR^{a9})R^{a9}, —NR^{a9}S(═O)₂R^{a9} and —S(═O)₂N(R^{a9})₂, wherein R^{a9} is as defined herein.

In some embodiments, R⁹ is selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-10 membered heterocyclyl, —OR^{a9}, —N(R^{a9})₂, —C(═O)R^{a9}, —C(=)OR^{a9}, —NR^{a9}C(═O)R^{a9}, —NR^{a9}C(═O)OR^{a9}, —C(═O)N(R^{a9})₂, —OC(═O)N(R^{a9})₂, wherein R^{a9} is as defined herein.

In certain embodiments, R⁹ is selected from the group consisting of halo, —CN, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a9}, —N(R^{a9})₂, —C(═O)R^{a9} and —C(═O)N(R^{a9})₂ wherein R^{a9} is as defined herein.

In some embodiments, R⁹ is selected from the group consisting of of halo, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a9}, —C(═O)R^{a9} and —C(═O)N(R^{a9})₂, wherein R^{a9} is as defined herein.

In some embodiments, R⁹ is selected from the group consisting of —C₁-C₆ alkyl, 3-membered heterocyclyl (e.g., oxetanyl), —C₃-C₉ cycloalkyl (e.g., cyclopropyl) and —C(═O)N(R^{a9})₂, wherein each R^{a9} is as defined in any of the embodiments described herein. In some embodiments, each R^{a9} is independently selected from the group consisting of H and —C₁-C₆ alkyl (e.g., -Me, -Et, —Pr, -iPr, -nBu, -tBu, -sec-Bu, -iso-Bu).

In some embodiments, R⁹ is selected from the group consisting of —C₁, -Me, -Et, —ⁱPr, —CF₃, —CHF₂, —OCHF₂, —OCF₃, cyclopropyl, —OCH₃, oxetan-3-yl, tetrahydrofuran-3-yl, —C(═O)NHOH, —C(═O)H and —C(═O)NH₂.

In certain embodiments, R⁹ is selected from the group consisting of —C₁-C₆ alkyl (e.g., -Me, -Et, —Pr, -iPr, -nBu, -tBu, -sec-Bu, -iso-Bu), 3-10 membered heterocyclyl (e.g., oxetan-3-yl), —C₃-C₉ cycloalkyl (e.g., cyclopropyl) and —C(═O)NH₂.

In some embodiments, R⁹ is selected from the group consisting of —C₁, -Me, -Et, —ⁱPr, —CF₃, —CHF₂, —OCHF₂, —OCF₃, and cyclopropyl. In some embodiments, R⁹ is selected from the group consisting of cyclopropyl, -Me and -Et.

In some embodiments, R⁹ is selected from the group consisting of -Me, -Et, oxetan-3-yl, cyclopropyl and —C(═O)NH₂.

In some embodiments, R⁹ is selected from the group consisting of -Me, -Et, oxetan-3-yl and cyclopropyl.

In some embodiments, R⁹ is H. In some embodiments, R⁹ is D.

In certain embodiments, R⁹ is halo (e.g., fluoro, chloro, bromo, iodo). In some embodiments, R⁹ is —Cl. In some embodiments, R⁹ is —F. In some embodiments, R⁹ is —Br. In some embodiments, R⁹ is —I.

In some embodiments, R⁹ is —CN.

In certain embodiments, R⁹ is —C₁-C₆ alkyl. In some embodiments, R⁹ is -Me. In some embodiments, R⁹ is -Et. In some embodiments R⁹ is —Pr or -iPr.

In some embodiments, R⁹ is —C₁-C₆ heteroalkyl. In some embodiments, R⁹ is methoxymethyl (—CH₂OCH₃). In some embodiments, R⁹ is hydroxymethyl (—CH₂OH). In some embodiments, R⁹ is aminomethyl (e.g., —CH₂NH₂, —CH₂NHCH₃, —CH₂N(CH₃)₂.

In some embodiments, R⁹ is —C₁-C₆ haloalkyl. In some embodiments, R⁹ is trifluoromethyl (—CF₃). In other embodiments, R⁹ is difluoromethyl (—CHF₂).

In some embodiments, R⁹ is —C₃-C₉ cycloalkyl (e.g., cyclopropyl, cyclobutyl, cyclopentyl, cyclohexyl). In some embodiments, R⁹ is cyclopropyl. In some embodiments R⁹ is cyclobutyl. In some embodiments, R⁹ is cyclopentyl. In some embodiments, R⁹ is cyclohexyl.

In some embodiments, R⁹ is 3-6 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R⁹ is oxetanyl (e.g., oxetan-3-yl). In some embodiments, R⁹ is tetrahydropyranyl. In some embodiments, R⁹ is tetrahydrofuranyl. In some embodiments, R⁹ is azetidinyl. In some embodiments, R⁹ is pyrrolidinyl. In some embodiments, R⁹ is piperidinyl. In some embodiments, R⁹ is piperazinyl. In some embodiments, R⁹ is morpholinyl. In some embodiments, R⁹ is azepanyl.

In some embodiments R⁹ is cycloalkylalkyl (e.g., cyclopropylmethyl, cyclobutylmethyl, cyclopentylmethyl, cyclohexylmethyl, cycloheptylmethyl). In some embodiments, R⁹ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R⁹ is arylalkyl. In some embodiments, R⁹ is benzyl.

In some embodiments, R⁹ is heteroarylalkyl (e.g., pyridinylmethyl, thiazolylmethyl, triazolylmethyl, pyrazolylmethyl).

In some embodiments, R⁹ is —OR^{a9} wherein R^{a9} is as defined in any of the embodiments described herein (e.g., hydroxy (—OH), methoxy, ethoxy, propoxy, isopropoxy, cyclopropyloxy, cyclobutyloxy). In some embodiments, R⁹ is hydroxy. In some embodiments, R⁹ is methoxy. In some embodiments, R⁹ is ethoxy. In some embodiments, R⁹ is propoxy. In some embodiments, R⁹ is isopropoxy. In some embodiments, R⁹ is —C₁-C₆ haloalkoxy. In some embodiments, R⁹ is trifluoromethoxy (—OCF₃), In other embodiments, R⁹ is difluoromethoxy (—OCHF₂).

In some embodiments, R⁹ is —N(R^{a9})₂ wherein R^{a9} is as defined in any of the embodiments described herein (e.g., —NH₂, —NHR^{a9}, —N(CH₃)R^{a9}). In some embodiments, R⁹ is —NH₂. In some embodiments, R⁹ is —NHR^{a9} (e.g., —NHCH₃, -NHEt, —NHPr, —NHⁱPr, -NHcyclopropyl, -NHcyclobutyl). In some embodiments, R⁹ is —N(CH₃)R^{a9} (e.g., —N(CH₃)₂, —N(CH₃)Et, —N(CH₃)Pr, —N(CH₃)ⁱPr, —N(CH₃)cyclopropyl, —N(CH₃)cyclobutyl).

In some embodiments, R⁹ is —C(═O)R^{a9} or —C(═O)OR^{a9} wherein R^{a9} is as defined in any of the embodiments described herein. In some embodiments, R⁹ is —C(═O)R^{a9} wherein R^{a9} is as defined in any of the embodiments described herein. In some embodiments, R⁹ is —C(═O)alkyl. In some embodiments, R⁹ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R⁹ is acetyl (—C(═O)Me). In some embodiments, R⁹ is —C(═O)OR^{a9}. In some embodiments, R⁹ is —COOH. In some embodiments, R⁹ is COOCH₃.

In some embodiments, R⁹ is —NR^{a9}C(═O)R^{a9} wherein R^{a9} is as defined in any of the embodiments described herein. In certain embodiments, R⁹ is —NHC(═O)R^{a9} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R⁹ is —N(CH₃)C(═O)R^{a9} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R⁹ is —NR^{a9}C(═O)OR^{a9} wherein R^{a9} is as defined in any of the embodiments described herein. In certain embodiments, R⁹ is —NHC(═O)OR^{a9} (e.g., —NHC(═O)OCH₃, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R⁹ is —N(CH₃)C(═O)OR^{a9} (e.g., —N(CH₃)C(═O)OCH₃, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R⁹ is —C(═O)N(R^{a9})₂ wherein R^{a9} is as defined in any of the embodiments described herein (e.g., —C(═O)NH₂, —C(═O)NHR^{a9}, —C(═O)N(CH₃)R^{a9}). In some embodiments, R⁹ is —C(═O)NH₂. In certain embodiments, R⁹ is —C(═O)NHR^{a9} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁹ is —C(═O)N(CH₃)R^{a9} (e.g., —C(═O)N(CH₃)₂, —C(═O)N(CH₃)Et, —C(═O)N(CH₃)Pr, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R⁹ is —C(═O)N(OR^{a9})(R^{a9}) wherein R^{a9} is as defined in any of the embodiments described herein. In certain embodiments, R⁹ is —C(═O)NH(OR^{a9}) (e.g., —C(═O)NHOH, —C(═O)NHOCH3). In some embodiments, R⁹ is —C(═O)NHOH.

In some embodiments, R⁹ is —OC(═O)N(R^{a9})₂ wherein R^{a9} is as defined in any of the embodiments described herein. In certain embodiments, R⁹ is —OC(═O)NHR^{a9} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R⁹ is —OC(═O)N(CH₃)R^{a9} (e.g., —OC(═O)N(CH₃)₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R⁹ is —S(═O)R^{a9} wherein R^{a9} is as defined in any of the embodiments described herein. In certain embodiments, R⁹ is —S(═O)alkyl (e.g., —S(═O)Me, —S(═O)Et, —S(═O)Pr, —S(═O)ⁱPr). In certain embodiments, R⁹ is —S(═O)cycloalkyl (e.g., —S(═O)cyclopropyl, —S(═O)cyclobutyl, —S(═O)cyclopentyl, —S(═O)cyclohexyl).

In some embodiments, R⁹ is —S(═O)₂R^{a9} wherein R^{a9} is as defined in any of the embodiments described herein. In certain embodiments, R⁹ is —S(═O)₂alkyl (e.g., —S(═O)₂Me, —S(═O)₂Et, —S(═O)₂Pr, —S(═O)₂′Pr). In certain embodiments, R⁹ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁹ is S(═O)₂aryl (e.g., —S(═O)₂phenyl).

In some embodiments, R⁹ is —SR^{a9} wherein R^{a9} is as defined in any of the embodiments described herein. In certain embodiments, R⁹ is -Salkyl (e.g., —SMe, -SEt, —SPr, —SⁱPr). In certain embodiments, R⁹ is -Scycloalkyl (e.g., -Scyclopropyl, -Scyclobutyl, -Scyclopentyl, -Scyclohexyl). In certain embodiments, R⁹ is -Saryl (e.g., -Sphenyl).

In some embodiments, R⁹ is —S(═O)(═NR^{a9})R^{a9} wherein R^{a9} is as defined in any of the embodiments described herein. In certain embodiments, R⁹ is —S(═O)(═NH)R^{a9} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R⁹ is —S(═O)(═NCH₃)R^{a9} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R⁹ is —NR^{a9}S(═O)₂R^{a9} wherein R^{a9} is as defined in any of the embodiments described herein. In certain embodiments, R⁹ is —NHS(═O)₂alkyl (e.g., —NHS(═O)₂Me, —NHS(═O)₂Et, —NHS(═O)₂Pr, —NHS(═O)₂′Pr). In certain embodiments, R⁹ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R⁹ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R⁹ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R⁹ is —S(═O)₂N(R^{a9})₂ wherein R^{a9} is as defined in any of the embodiments described herein. (e.g., —S(═O)₂NH₂, —S(═O)₂NHR^{a9}, —S(═O)₂N(CH₃)R^{a9}). In some embodiments, R⁹ is —S(═O)₂NH₂. In some embodiments, R⁹ is —S(═O)₂NHR^{a9} (e.g., —S(═O)₂NHCH₃, —S(═O)₂NHEt, —S(═O)₂NHPr, —S(═O)₂NH′Pr, —S(═O)₂NHcyclopropyl, —S(═O)₂NHcyclobutyl). In some embodiments, R⁹ is —S(═O)₂N(CH₃)R^{a9} (e.g., —S(═O)₂N(CH₃)₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

Some embodiments of feature certain combinations ofR⁸ and R⁹. In one embodiment, R⁸ is selected from the group consisting of H, —OCH₃, -OEt, —OCF₃, —OCHF₂, —CHF₂, -Me, -Et, —OH and —NH₂ and R⁹ is selected from the group consisting of —C₁, -Me, -Et, —ⁱPr, —CF₃, —CHF₂, —OCHF₂, cyclopropyl and —C(═O)NH₂. In one embodiment, R⁸ is selected from the group consisting of H, —CHF₂, -Me and —NH₂ and R⁹ is selected from the group consisting of —C₁, -Me, -Et, —CF₃, —CHF₂, —OCHF₂, oxetan-3-yl and cyclopropyl. In a further embodiment, R⁸ is selected from the group consisting of —NH₂ and -Me and R⁹ is selected from the group consisting of -Me, -Et, oxetan-3-yl and cyclopropyl.

In one embodiment, R⁸ is —NH₂ and R⁹ is selected from the group consisting of -Me, -Et, oxetan-3-yl and cyclopropyl.

In another embodiment, R⁸ is selected from the group consisting of H, —OCH₃, -OEt, —OCF₃, —OCHF₂, -Et and —OH and R⁹ is —C(═O)NH₂. In some embodiments, R⁸ is selected from the group consisting of H and —OCH₃ and R⁹ is —C(═O)NH₂. In some embodiments, R⁸ is —OCH₃ and R⁹ is —C(═O)NH₂.

As generally described herein, each R¹⁰ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a10}, —N(R^{a10})₂, —C(═O)R^{a10}, —C(═O)OR^{a10}, —NR^{a10}C(═O)R^{a10}, —NR^{a10}C(═O)OR^{a10}, —C(═O)N(R^{a10})₂, —OC(═O)N(R^{a10})₂, —S(═O)R^{a10}, —S(═O)₂R^{a10}, —SR^{a10}, —S(═O)(═NR^{a10})R^{a10}, —NR^{a10}S(═O)₂R^{a10} and —S(═O)₂N(R^{a10})₂, wherein R^{a10} is as defined in any of the embodiments described herein.

In some embodiments, R¹⁰ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a10}, —N(R^{a10})₂—C(═O)R^{a10}, —C(═O)OR^{a10}, —NR^{a1}OC(═O)R^{a10}, NR^{a10}C(═O)OR^{a10}, —C(═O)N(R^{a10})₂ and —OC(═O)N(R^{a10}), wherein R^{a10} is as defined in any of the embodiments described herein.

In certain embodiments, R¹⁰ is selected from the group consisting of H, halo, —CN, —C₁-C₆ alkyl, —C₁-C₆ haloalkyl and —N(R^{a10})₂ wherein R^{a10} is as defined in any of the embodiments described herein. In some embodiments, R^{a1} is selected from the group consisting of H and —C₁-C₆ alkyl (e.g., -Me, -Et, —Pr, -iPr, -nBu, -tBu, -sec-Bu, -iso-Bu). In some embodiments, R¹⁰ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl) (e.g., —OCH₃), —NH₂, —NH—(C₁-C₆ alkyl) (e.g., —NHCH₃) and —N—(C₁-C₆ alkyl)₂ (e.g, —N(CH₃)₂). In some embodiments, R¹⁰ is selected from the group consisting of H, -Me and —NH₂. In certain embodiments, R¹⁰ is selected from the group consisting of H and -Me.

In some embodiments, R¹⁰ is H. In some embodiments R¹⁰ is -D.

In certain embodiments, R¹⁰ is halo (e.g., fluoro, chloro, bromo, iodo). In some embodiments, R¹⁰ is —Cl. In some embodiments, R¹⁰ is —F. In some embodiments, R¹⁰ is —Br. In some embodiments, R¹⁰ is —I.

In some embodiments, R¹⁰ is —CN.

In certain embodiments, R¹⁰ is —C₁-C₆ alkyl. In some embodiments, R¹⁰ is -Me. In some embodiments, R¹⁰ is -Et. In some embodiments R¹⁰ is —Pr or -iPr.

In some embodiments, R¹⁰ is —C₁-C₆ heteroalkyl. In some embodiments, R¹⁰ is methoxymethyl (—CH₂OCH₃). In some embodiments, R¹⁰ is hydroxymethyl (—CH₂OH). In some embodiments, R¹⁰ is aminomethyl (e.g., —CH₂NH₂, —CH₂NHCH₃, —CH₂N(CH₃)₂.

In some embodiments, R¹⁰ is —C₁-C₆ haloalkyl. In some embodiments, R¹⁰ is trifluoromethyl (—CF₃). In other embodiments, R¹⁰ is difluoromethyl (—CHF₂).

In some embodiments, R¹⁰ is —C₃-C₉ cycloalkyl (e.g., cyclopropyl, cyclobutyl, cyclopentyl, cyclohexyl). In some embodiments, R¹⁰ is cyclopropyl. In some embodiments R¹⁰ is cyclobutyl. In some embodiments, R¹⁰ is cyclopentyl. In some embodiments, R¹⁰ is cyclohexyl.

In some embodiments, R¹⁰ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R¹⁰ is oxetanyl. In some embodiments, R¹⁰ is tetrahydropyranyl. In some embodiments, R¹⁰ is tetrahydrofuranyl. In some embodiments, R¹⁰ is azetidinyl. In some embodiments, R¹⁰ is pyrrolidinyl. In some embodiments, R¹⁰ is piperidinyl. In some embodiments, R¹⁰ is piperazinyl. In some embodiments, R¹⁰ is morpholinyl. In some embodiments, R¹⁰ is azepanyl.

In some embodiments R¹⁰ is cycloalkylalkyl (e.g., cyclopropylmethyl, cyclobutylmethyl, cyclopentylmethyl, cyclohexylmethyl, cycloheptylmethyl). In some embodiments, R¹⁰ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R¹⁰ is arylalkyl. In some embodiments, R¹⁰ is benzyl.

In some embodiments, R¹⁰ is heteroarylalkyl (e.g., pyridinylmethyl, thiazolylmethyl, triazolylmethyl, pyrazolylmethyl).

In some embodiments, R¹⁰ is —OR^{a10} wherein R^{a10} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R¹⁰ is hydroxy. In some embodiments, R¹⁰ is methoxy. In some embodiments, R¹⁰ is ethoxy. In some embodiments, R¹⁰ is propoxy. In some embodiments, R¹⁰ is isopropoxy. In some embodiments R¹⁰ is difluoromethoxy. (—OCHF₂). In some embodiments, R¹⁰ is trifluoromethoxy (—OCF₃).

In some embodiments, R¹⁰ is —N(R^{a10})₂ wherein R^{a10} is as defined in any of the embodiments described herein (e.g., —NH₂, —NHR^{a10}, —N(CH₃)R^{a10}). In some embodiments, R¹⁰ is —NH₂. In some embodiments, R¹⁰ is -NHR^{a1} (e.g., —NHCH₃, -NHEt, —NHPr, —NHⁱPr, -NHcyclopropyl, -NHcyclobutyl). In some embodiments, R¹⁰ is —N(CH₃)R^{a1} (eg, —N(CH₃)₂, —N(CH₃)Et, —N(CH₃)Pr, —N(CH₃)ⁱPr, —N(CH₃)cyclopropyl, —N(CH₃)cyclobutyl).

In some embodiments, R¹⁰ is —C(═O)R^{a0} or —C(═O)OR^{a1} wherein R^{a10} is as defined in any of the embodiments described herein. In some embodiments, R¹⁰ is —C(═O)Rao wherein R^{a10} is as defined in any of the embodiments described herein. In some embodiments, R¹⁰ is —C(═O)alkyl. In some embodiments, R¹⁰ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R¹⁰ is acetyl (—C(═O)Me). In some embodiments, R¹⁰ is —C(═O)OR^{a10}In some embodiments, R¹⁰ is —COOH. In some embodiments, R¹⁰ is COOCH₃.

In some embodiments, R¹⁰ is —NR^{a10}C(═O)R^{a10} wherein R^{a10} is as defined in any of the embodiments described herein. In certain embodiments, R¹⁰ is —NHC(═O)R^{a10} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R¹⁰ is —N(CH₃)C(═O)R^{a10} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R¹⁰ is —NR^{a10}C(═O)OR^{a10} wherein R^{a10} is as defined in any of the embodiments described herein. In certain embodiments, R¹⁰ is —NHC(═O)OR^{a10} (e.g., —NHC(═O)OCH₃, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R¹⁰ is —N(CH₃)C(═O)OR^{a10} (e.g., —N(CH₃)C(═O)OCH₃, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R¹⁰ is —C(═O)N(R^{a10})₂wherein R^{a10} is as defined in any of the embodiments described herein (e.g., —C(═O)NH₂, —C(═O)NHR^{a10}, —C(═O)N(CH₃)R^{a10}) In some embodiments, R¹⁰ is —C(═O)NH₂. In certain embodiments, R¹⁰ is —C(═O)NHR^{a10} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R¹° is —C(═O)N(CH₃)R^{a10} (e.g., —C(═O)N(CH₃)₂, —C(═O)N(CH₃)Et, —C(═O)N(CH₃)Pr, —C(═O)N(CH₃)ⁱPr, —C(═O)N(CH₃)Bu, —C(═O)N(CH₃)Bu, —C(═O)N(CH₃)Cyclopropyl, —C(═O)N(CH₃)Cyclobutyl).

In some embodiments, R¹⁰ is —OC(═O)N(R^{a10})₂ wherein R^{a10} is as defined in any of the embodiments described herein. In certain embodiments, R¹⁰ is —OC(═O)NHR^{a10} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R¹⁰ is —OC(═O)N(CH₃)R^{a1} (e.g., —OC(═O)N(CH₃)₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)Bu, —OC(═O)N(CH₃)Cyclopropyl, —OC(═O)N(CH₃)Cyclobutyl).

In some embodiments, R¹⁰ is —S(═O)R^{a10} wherein R^{a10} is as defined in any of the embodiments described herein. In certain embodiments, R¹⁰ is —S(═O)alkyl (e.g., —S(═O)Me, —S(═O)Et, —S(═O)Pr, —S(═O)ⁱPr). In certain embodiments, R¹⁰ is —S(═O)cycloalkyl (e.g., —S(═O)cyclopropyl, —S(═O)cyclobutyl, —S(═O)cyclopentyl, —S(═O)cyclohexyl).

In some embodiments, R¹⁰ is —S(═O)₂R^{a10} wherein R^{a10} is as defined in any of the embodiments described herein. In certain embodiments, R¹⁰ is —S(═O)₂alkyl (e.g., —S(═O)₂Me, —S(═O)₂Et, —S(═O)₂Pr, —S(═O)₂′Pr). In certain embodiments, R¹⁰ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R¹⁰ is S(═O)₂aryl (e.g., —S(═O)₂phenyl).

In some embodiments, R¹⁰ is —SR^{a10} wherein R^{a10} is as defined in any of the embodiments described herein. In certain embodiments, R¹⁰ is -Salkyl (e.g., —SMe, -SEt, —SPr, —SⁱPr). In certain embodiments, R¹⁰ is -Scycloalkyl (e.g., -Scyclopropyl, -Scyclobutyl, -Scyclopentyl, -Scyclohexyl). In certain embodiments, R¹⁰ is -Saryl (e.g., -Sphenyl).

In some embodiments, R¹⁰ is —S(═O)(═NR^{a10})R^{a10} wherein R^{a10} is as defined in any of the embodiments described herein. In certain embodiments, R¹⁰ is —S(═O)(═NH)R^{a10} (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R¹⁰ is —S(═O)(═NCH₃)R^{a10} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R¹⁰ is —NR^{a10}S(═O)₂R^{a10} wherein R^{a10} is as defined in any of the embodiments described herein. In certain embodiments, R¹⁰ is —NHS(═O)₂alkyl (e.g., —NHS(═O)₂Me, —NHS(═O)₂Et, —NHS(═O)₂Pr, —NHS(═O)₂′Pr). In certain embodiments, R¹⁰ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R¹⁰ is —N(CH₃)S(═O)₂alkyl (e.g., —N(CH₃)S(═O)₂Me, —N(CH₃)S(═O)₂Et, —N(CH₃)S(═O)₂Pr, —N(CH₃)S(═O)₂′Pr). In certain embodiments, R¹⁰ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R¹⁰ is —S(═O)₂N(R^{a10})₂ wherein R^{a10} is as defined in any of the embodiments described herein. (e.g., —S(═O)₂NH₂, —S(═O)₂NHR^{a10}, —S(═O)₂N(CH₃)R^{a10}) In some embodiments, R¹⁰ is —S(═O)₂NH₂. In some embodiments, R¹⁰ is —S(═O)₂NHR^{a10} (e.g., —S(═O)₂NHCH₃, —S(═O)₂NHEt, —S(═O)₂NHPr, —S(═O)₂NH′Pr, —S(═O)₂NHcyclopropyl, —S(═O)₂NHcyclobutyl). In some embodiments, R¹⁰ is —S(═O)₂N(CH₃)R^{a10} (e.g., —S(═O)₂N(CH₃)₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally described herein, each R¹¹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a11}, —N(Ra)₂, —C(═O)R^{a11}, —C(═O)OR^{a11}, —NRa¹¹C(═O)R^{a11}, —NR^{a11}C(═O)OR^{a1}, —C(═O)N(R^{a1})₂, —OC(═O)N(Ra)₂, —S(═O)R^{a11}, —S(═O)₂R^{a11}, —SR^{a1}, —S(═O)(═NR^{a11})R^{a1}, —NR^{a11}S(═O)₂R^{a11} and —S(═O)₂N(Ra)₂, wherein each R^{a11} is as defined in any of the embodiments described herein.

In certain embodiments, R¹¹ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a11}, —N(Ra)₂, —C(═O)R^{a11}, —C(═O)OR^{a11}, —NR^{a11}C(═O)R^{a1}, NR^{a11}C(═O)OR^{a11}, —C(═O)N(Ra)₂ and —OC(═O)N(Ra)₂, wherein each R^{a11} is as defined in any of the embodiments described herein.

In some embodiments, R¹¹ is selected from the group consisting of H, halo, —CN, —C₁-C₆ alkyl, —C₁-C₆ haloalkyl and —N(Ra)₂, wherein each R^{a11} is as defined in any of the embodiments described herein. In some embodiments, each R^{a11} is independently selected from the group consisting of H and —C₁-C₆ alkyl (e.g., -Me, -Et, —Pr, -iPr, -nBu, -tBu, -sec-Bu, -iso-Bu). In some embodiments, R¹¹ is selected from the group consisting of —C₁-C₆ alkyl (e.g., -Me, -Et, —Pr, -iPr, -nBu, -tBu, -sec-Bu, -iso-Bu), —OH, —O—(C₁-C₆ alkyl) (e.g., —OCH₃), —NH₂, —NH—(C₁-C₆ alkyl) (e.g., —NHCH₃) and —N—(C₁-C₆ alkyl)₂ (e.g, —N(CH₃)₂).

In some embodiments, R¹¹ is H. In some embodiments R¹¹ is -D.

In certain embodiments, R¹¹ is halo (e.g., fluoro, chloro, bromo, iodo). In some embodiments, R¹¹ is —Cl. In some embodiments, R¹¹ is —F. In some embodiments, R¹¹ is —Br.

In some embodiments, R¹¹ is —I.

In some embodiments, R¹¹ is —CN.

In certain embodiments, R¹¹ is —C₁-C₆ alkyl. In some embodiments, R⁵ⁱ is -Me. In some embodiments, R¹¹ is -Et. In some embodiments R¹¹ is —Pr or -iPr.

In some embodiments, R¹¹ is —C₁-C₆ heteroalkyl. In some embodiments, R⁵ⁱ is methoxymethyl (—CH₂OCH₃). In some embodiments, R¹¹ is hydroxymethyl (—CH₂OH). In some embodiments, R¹¹ is aminomethyl (e.g., —CH₂NH₂, —CH₂NHCH₃, —CH₂N(CH₃)₂.

In some embodiments, R¹¹ is —C₁-C₆ haloalkyl. In some embodiments, R¹¹ is trifluoromethyl (—CF₃). In other embodiments, R⁵¹ is difluoromethyl (—CHF₂). In some embodiments, R¹¹ is —C₃-C₉ cycloalkyl (e.g., cyclopropyl, cyclobutyl, cyclopentyl, cyclohexyl). In some embodiments, R¹¹ is cyclopropyl. In some embodiments R¹¹ is cyclobutyl. In some embodiments, R¹¹ is cyclopentyl. In some embodiments, R¹¹ is cyclohexyl.

In some embodiments, R¹¹ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R¹¹ is oxetanyl. In some embodiments, R¹¹ is tetrahydropyranyl. In some embodiments, R¹ is tetrahydrofuranyl. In some embodiments, R¹ is azetidinyl. In some embodiments, R¹¹ is pyrrolidinyl. In some embodiments, R¹¹ is piperidinyl. In some embodiments, R¹¹ is piperazinyl. In some embodiments, R¹¹ is morpholinyl. In some embodiments, R¹¹ is azepanyl.

In some embodiments R¹¹ is cycloalkylalkyl (e.g., cyclopropylmethyl, cyclobutylmethyl, cyclopentylmethyl, cyclohexylmethyl, cycloheptylmethyl). In some embodiments, R¹¹ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R¹ is arylalkyl. In some embodiments, R¹¹ is benzyl. In some embodiments, R¹¹ is heteroarylalkyl (e.g., pyridinylmethyl, thiazolylmethyl, triazolylmethyl, pyrazolylmethyl).

In some embodiments, R¹ is —OR^{a11} wherein R^{a11} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R¹¹ is hydroxy. In some embodiments, R¹¹ is methoxy. In some embodiments, R¹¹ is ethoxy. In some embodiments, R¹ is propoxy. In some embodiments, R¹¹ is isopropoxy. In some embodiments R¹¹ is difluoromethoxy. (—OCHF₂). In some embodiments, R¹¹ is trifluoromethoxy (—OCF₃).

In some embodiments, R¹¹ is —N(Ra)₂ wherein R^{a11} is as defined in any of the embodiments described herein (e.g., —NH₂, —NHR^{a11}, —N(CH₃)Ra). In some embodiments, R¹¹ is —NH₂. In some embodiments, R¹¹ is —NHR^{a11} (e.g., —NHCH₃, -NHEt, —NHPr, —NHⁱPr, -NHcyclopropyl, -NHcyclobutyl). In some embodiments, R¹¹ is —N(CH₃)R^{a11} (eg, —N(CH₃)₂, —N(CH₃)Et, —N(CH₃)Pr, —N(CH₃)ⁱPr, —N(CH₃)cyclopropyl, —N(CH₃)cyclobutyl).

In some embodiments, R⁵ⁱ is —C(═O)R^{a11} or —C(═O)OR^{a11} wherein R^{a11} is as defined in any of the embodiments described herein. In some embodiments, R¹¹ is —C(═O)Ran wherein R^{a11} is as defined in any of the embodiments described herein. In some embodiments, R¹¹ is —C(═O)alkyl. In some embodiments, R¹¹ is —C(═O)CH₃, —C(═O)cyclopropyl, —C(═O)cyclobutyl, —C(═O)^{t}Bu, —C(═O)ⁱPr, —C(═O)Pr or —C(═O)OCH₃. In some embodiments, R¹¹ is acetyl (—C(═O)Me). In some embodiments, R¹¹ is —C(═O)OR^{a11} In some embodiments, R¹¹ is —COOH. In some embodiments, R¹¹ is COOCH₃.

In some embodiments, R¹¹ is —NR^{a11}C(═O)R^{a11} wherein R^{a11} is as defined in any of the embodiments described herein. In certain embodiments, R¹¹ is —NHC(═O)R^{a11} (e.g., —NHC(═O)Me, —NHC(═O)Et, —NHC(═O)Pr, —NHC(═O)ⁱPr, —NHC(═O)Bu, —NHC(═O)^{t}Bu, —NHC(═O)Cyclopropyl, —NHC(═O)Cyclobutyl). In some embodiments, R¹¹ is —N(CH₃)C(═O)R^{a11} (e.g., —N(CH₃)C(═O)Me, —N(CH₃)C(═O)Et, —N(CH₃)C(═O)Pr, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R¹¹ is —NR^{a11}C(═O)OR^{a11} wherein R^{a11} is as defined in any of the embodiments described herein. In certain embodiments, R¹¹ is —NHC(═O)OR^{a11} (e.g., —NHC(═O)OCH₃, —NHC(═O)OEt, —NHC(═O)OPr, —NHC(═O)OⁱPr, —NHC(═O)OBu, —NHC(═O)O^{t}Bu, —NHC(═O)OCyclopropyl, —NHC(═O)OCyclobutyl). In some embodiments, R¹¹ is —N(CH₃)C(═O)OR^{a11} (e.g., —N(CH₃)C(═O)OCH₃, —N(CH₃)C(═O)OEt, —N(CH₃)C(═O)OPr, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R¹¹ is —C(═O)N(R^{a11})₂ wherein R^{a11} is as defined in any of the embodiments described herein (e.g., —C(═O)NH₂, —C(═O)NHR^{a11}, —C(═O)N(CH₃)R^{a11}) In some embodiments, R¹¹ is —C(═O)NH₂. In certain embodiments, R¹¹ is —C(═O)NHR^{a11} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R¹¹ is —C(═O)N(CH₃)R^{a11} (e.g., —C(═O)N(CH₃)₂, —C(═O)N(CH₃)Et, —C(═O)N(CH₃)Pr, —C(═O)N(CH₃)ⁱPr, —C(═O)N(CH₃)Bu, —C(═O)N(CH₃) #Bu, —C(═O)N(CH₃)Cyclopropyl, —C(═O)N(CH₃)Cyclobutyl).

In some embodiments, R¹¹ is —OC(═O)N(R^{a11})₂ wherein R^{a11} is as defined in any of the embodiments described herein. In certain embodiments, R¹¹ is —OC(═O)NHR^{a11} (e.g., —OC(═O)NHCH₃, —OC(═O)NHEt, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R¹¹ is —OC(═O)N(CH₃)R^{a11} (e.g., —OC(═O)N(CH₃)₂, —OC(═O)N(CH₃)Et, —OC(═O)N(CH₃)Pr, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R¹¹ is —S(═O)R^{a11} wherein R^{a11} is as defined in any of the embodiments described herein. In certain embodiments, R¹¹ is —S(═O)alkyl (e.g., —S(═O)Me, —S(═O)Et, —S(═O)Pr, —S(═O)ⁱPr). In certain embodiments, R¹¹ is —S(═O)cycloalkyl (e.g., —S(═O)cyclopropyl, —S(═O)cyclobutyl, —S(═O)cyclopentyl, —S(═O)cyclohexyl).

In some embodiments, R¹¹ is —S(═O)₂R^{a11} wherein R^{a11} is as defined in any of the embodiments described herein. In certain embodiments, R¹¹ is —S(═O)₂alkyl (e.g., —S(═O)₂Me, —S(═O)₂Et, —S(═O)₂Pr, —S(═O)₂′Pr). In certain embodiments, R⁵ⁱ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R¹¹ is S(═O)₂aryl (e.g., —S(═O)₂phenyl).

In some embodiments, R¹¹ is —SR^{a11} wherein R^{a11} is as defined in any of the embodiments described herein. In certain embodiments, R¹¹ is -Salkyl (e.g., —SMe, -SEt, —SPr, —SⁱPr). In certain embodiments, R¹¹ is -Scycloalkyl (e.g., -Scyclopropyl, -Scyclobutyl, -Scyclopentyl, -Scyclohexyl). In certain embodiments, R¹¹ is -Saryl (e.g., -Sphenyl).

In some embodiments, R¹¹ is —S(═O)(═NR^{a11})R^{a11} wherein R^{a11} is as defined in any of the embodiments described herein. In certain embodiments, R¹¹ is —S(═O)(═NH)Ra (e.g., —S(═O)(═NH)Me, —S(═O)(═NH)Et, —S(═O)(═NH)Pr, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R¹¹ is —S(═O)(═NCH₃)R^{a11} (e.g., —S(═O)(═NCH₃)Me, —S(═O)(═NCH₃)Et, —S(═O)(═NCH₃)Pr, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R¹¹ is —NR^{a11}S(═O)₂R^{a11} wherein R^{a11} is as defined in any of the embodiments described herein. In certain embodiments, R¹¹ is —NHS(═O)₂alkyl (e.g., —NHS(═O)₂Me, —NHS(═O)₂Et, —NHS(═O)₂Pr, —NHS(═O)₂′Pr). In certain embodiments, R¹¹ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl).

In certain embodiments, R¹¹ is —N(CH₃)S(═O)₂alkyl wherein R^{a11} is as defined in any of the embodiments described herein (e.g., —N(CH₃)S(═O)₂Me, —N(CH₃)S(═O)₂Et, —N(CH₃)S(═O)₂Pr, —N(CH₃)S(═O)₂′Pr). In certain embodiments, R¹¹ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R¹¹ is —S(═O)₂N(R^{a11})₂ wherein R^{a11} is as defined in any of the embodiments described herein. (e.g., —S(═O)₂NH₂, —S(═O)₂NHR^{a11}, —S(═O)₂N(CH₃)Ra) In some embodiments, R¹¹ is —S(═O)₂NH₂. In some embodiments, R¹¹ is —S(═O)₂NHR^{a11} (e.g., —S(═O)₂NHCH₃, —S(═O)₂NHEt, —S(═O)₂NHPr, —S(═O)₂NH′Pr, —S(═O)₂NHcyclopropyl, —S(═O)₂NHcyclobutyl). In some embodiments, R¹¹ is —S(═O)₂N(CH₃)Ra (e.g., —S(═O)₂N(CH₃)₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

In some embodiments, Ring A is selected from the group consisting of:

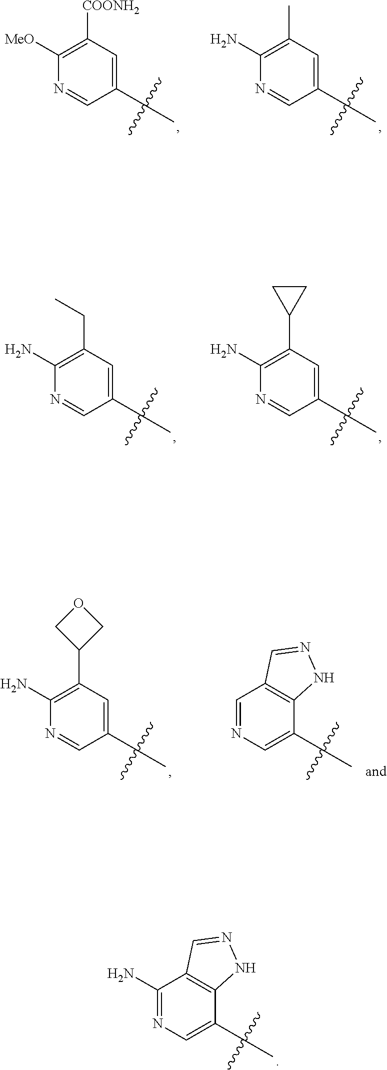

As generally described herein, Ring B is selected from the group consisting of C₆-C₁₀ aryl and 5-10 membered heteroaryl, each optionally substituted at any available position.

In some embodiments, each aryl and heteroaryl of Ring B is substituted at any available position with 0, 1, 2 or 3 instances of R³, wherein each R³ is as defined in any of the embodiments described herein.

In some embodiments, Ring B is independently selected from the group consisting of a-C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof), 5-6 membered monocyclic heteroaryl (e.g., containing 1-4 heteroatoms independently selected from the group consisting of N, O and S) and an 8-10 membered bicyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R³ wherein R³ is as defined in any of the embodiments described herein).

In some embodiments, Ring B is selected from the group consisting of C₆-C₁₀ mono or bicyclic aryl (e.g., phenyl, naphthyl, 1,2,3,4-tetrahydronaphthalenyl, 2,3-dihydro-1H-indenyl, 1,2,3,4 tetrahydroquinolinyl, 1,2 dihydroquinolinyl, 1,2-dihydroisoquinolinyl, 1,2,3,4 tetrahydroisoquinolinyl, chromanyl, indolinyl, isoindolinyl, 3,4-dihydro-2H-benzo[b][1,4]oxazinyl, 2,3-dihydrobenzofuranyl, benzo[d][1,3]dioxolyl, 2,3-dihydro-1H-benzo[d]imidazolyl), 5-6 membered monocyclic heteroaryl (e.g., thiophenyl, thiazolyl, pyrazolyl, imidazolyl, oxazolyl, pyridinyl, pyrimidinyl), 8-10 membered bicyclic heteroaryl (e.g., benzo[d]isothiazolyl, indolyl, benzofuranyl, 1H-indazolyl, 2-H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, isoquinolinyl, benzo[d]imidazolyl, benzo[d]thiazolyl, benzo[d]oxazolyl, [1,2,4]triazolo[4,3-a]pyridinyl, imidazo[1,2-a]pyridinyl, imidazo[1,5-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl) wherein each aryl and heteroaryl is optionally substituted (e.g., substituted with 0, 1, 2 or 3 instances of R³ wherein R³ is as defined in any of the embodiments described herein).

In some embodiments, Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl), 1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each optionally substituted (e.g., substituted with 0, 1, 2 or 3 instances of R³ wherein R³ is as defined in any of the embodiments described herein).

In some embodiments, Ring B is selected from the group consisting of thiophenyl, phenyl and benzo[d]thiazolyl, each optionally substituted (e.g., substituted with 0, 1, 2 or 3 instances of R³ wherein R³ is as defined in any of the embodiments described herein).

In some embodiments, Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted (e.g., substituted with 0, 1, 2 or 3 instances of R³ wherein R³ is as defined in any of the embodiments described herein).

In some embodiments, Ring B is selected from the group consisting of thiophen-2-yl, thiophen-3-yl, phenyl, benzo[d]thiazol-5-yl, each optionally substituted (e.g., substituted with 0, 1, 2 or 3 instances of R³ wherein R³ is as defined in any of the embodiments described herein).

In some embodiments, Ring B is independently selected from the group consisting of a-C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof) and an 8-10 membered bicyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R³ wherein R³ is as defined in any of the embodiments described herein).

In some embodiments, Ring B is independently selected from the group consisting phenyl and an 8-10 membered bicyclic heteroaryl (e.g., containing 1-4 heteroatoms independently selected from the group consisting of N, O and S) wherein the phenyl and the heteroaryl is optionally substituted (e.g., substituted with 0, 1, 2 or 3 instances of R³ wherein R³ is as defined in any of the embodiments described herein).

In some embodiments, Ring B is selected from the group consisting of phenyl and benzo[d]thiazolyl, each optionally substituted (e.g., substituted with 0, 1, 2 or 3 instances of R³ wherein R³ is as defined in any of the embodiments described herein). In some embodiments, Ring B is selected from the group consisting of phenyl and benzo[d]thiazol-5-yl, each optionally substituted (e.g., substituted with 0, 1, 2 or 3 instances of R³ wherein R³ is as defined in any of the embodiments described herein).

In some embodiments, Ring B is independently selected from the group consisting of a-C₆-C₁₀ mono or bicyclic aryl (e.g., phenyl, fully aromatic 9-10 membered bicyclic aryl, bicyclic aryl containing a phenyl ring fused with a C₅-C₆ carbocycle, bicyclic aryl containing a phenyl ring fused with a 5-6 membered heterocycle containing 1-3 heteroatoms independently selected from the group consisting of N, O and S or oxidized forms thereof) and a 5-6 membered monocyclic heteroaryl (e.g., containing 1-4 heteroatoms independently selected from the group consisting of N, O and S) wherein each aryl and heteroaryl is optionally substituted (e.g., substituted with 0, 1, 2 or 3 instances of R³ wherein R³ is as defined in any of the embodiments described herein).

In some embodiments, Ring B is independently selected from the group consisting phenyl and a 5-6 membered monocyclic heteroaryl (e.g., containing 1-4 heteroatoms independently selected from the group consisting of N, O and S) wherein the phenyl and the heteroaryl is optionally substituted (e.g., substituted with 0, 1, 2 or 3 instances of R³ wherein R³ is as defined in any of the embodiments described herein).

In some embodiments, Ring B is selected from the group consisting of thiophenyl and phenyl, each optionally substituted (e.g., substituted with 0, 1, 2 or 3 instances of R³ wherein R³ is as defined in any of the embodiments described herein). In some embodiments, Ring B is selected from the group consisting of thiophen-2-yl, thiophen-3-yl and phenyl, each optionally substituted (e.g., substituted with 0, 1, 2 or 3 instances of R³ wherein R³ is as defined in any of the embodiments described herein).

In some embodiments, Ring B is unsubstituted. In some embodiments, Ring B is substituted with 1 instance of R³. In some embodiments, Ring B is substituted with 2 instances of R³. In some embodiments, Ring B is substituted with 3 instances of R³.

In some embodiments, Ring B is selected from the group consisting of:

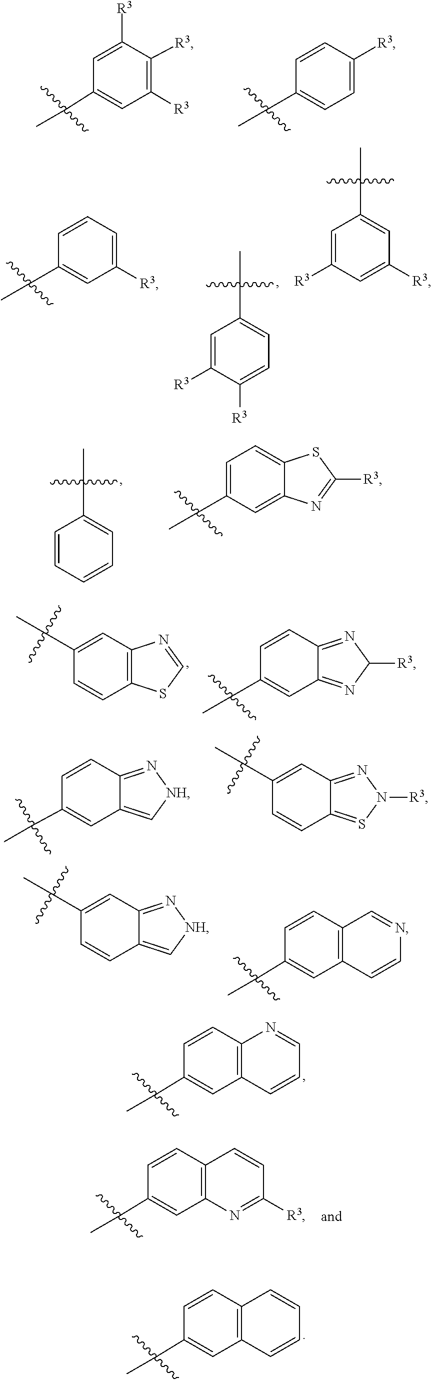

In some embodiments, Ring B is selected from the group consisting of

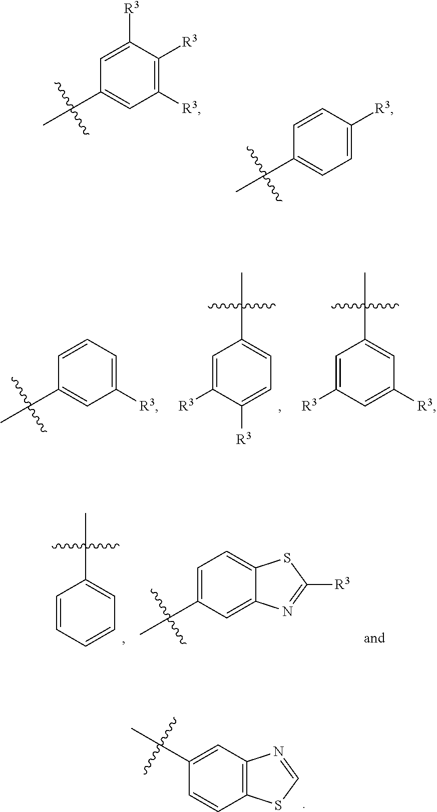

In some embodiments, Ring B is an optionally substituted 6-10 membered mono or bicyclic aryl. In some embodiments, Ring B is substituted with 0, 1, 2 or 3 instances of R³, wherein R³ is as defined in any of the embodiments described herein.

In some embodiments, Ring B is optionally substituted phenyl. In some embodiments, Ring B is phenyl substituted with 0, 1, 2 or 3 instances of R³, wherein each R³ is independently as defined in any of the embodiments described herein. In some embodiments, the phenyl is unsubstituted. In some embodiments, the phenyl is substituted with one instance of R. In some embodiments, the phenyl is substituted with 1 instance of R³ at the position para- to the attachment point to the piperidine. In some embodiments, the phenyl is substituted with 1 instance of R³ at the position meta- to the attachment point to the piperidine. In some embodiments, the phenyl is substituted with 2 instances of R³. In some embodiments, the phenyl is substituted with 3 instances of R³.

In some embodiments, Ring B is selected from the group consisting of

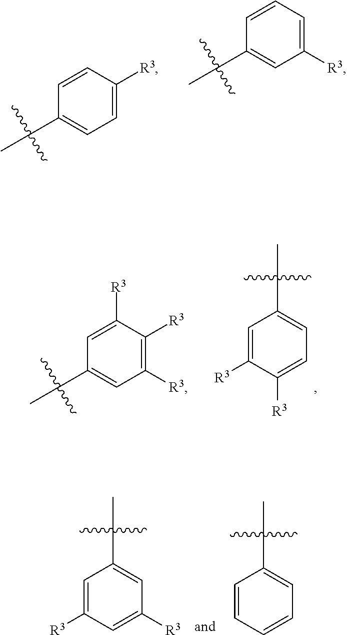

wherein each R³ is as defined herein.

In some embodiments, Ring B is

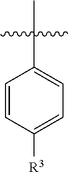

wherein each R³ is as defined herein.

In some embodiments, Ring B is

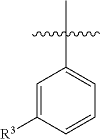

wherein each R³ is as defined herein.

In some embodiments, Ring B is

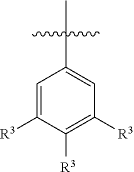

wherein each R³ is as defined herein.

In some embodiments, Ring B is

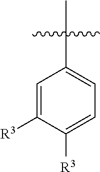

wherein each R³ is as defined herein.

In some embodiments, Ring B is

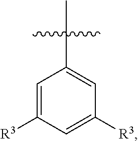

wherein each R³ is as defined herein.

In some embodiments, Ring B is

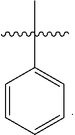 [SMILES: *c1ccccc1]

In some embodiments, the compounds of Formula (I) are of Formula (IV):

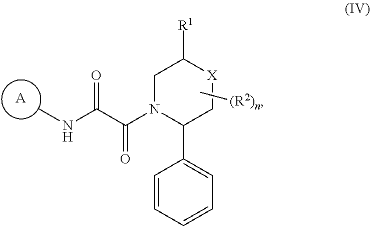

wherein X, Ring A, R¹, R² and n are as defined herein and the phenyl is substituted with 0, 1, 2 or 3 instances of R³ as defined herein. In some embodiments, the compounds of Formula (I) are of Formula (IVa):

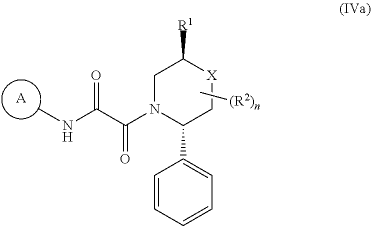

wherein X, Ring A, R¹, R² and n are as defined herein and the phenyl is substituted with 0, 1, 2 or 3 instances of R³ as defined herein. In some embodiments, the phenyl is unsubstituted. In some embodiments, the phenyl is substituted with one instance of R³. In some embodiments, the phenyl is substituted with 1 instance of R³ at the position para- to the attachment point to the piperidine. In some embodiments, the phenyl is substituted with 1 instance of R³ at the position meta- to the attachment point to the piperidine. In some embodiments, the phenyl is substituted with 2 instances of R³. In some embodiments, the phenyl is substituted with 3 instances of R.

In yet some embodiments, compounds of Formula (I) are of Formula (IV_1),

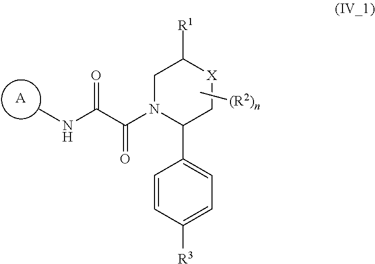

wherein X, Ring A, R¹, R² R³ and n are as defined herein.

In yet some embodiments, compounds of Formula (I) are of Formula (IV_1a),

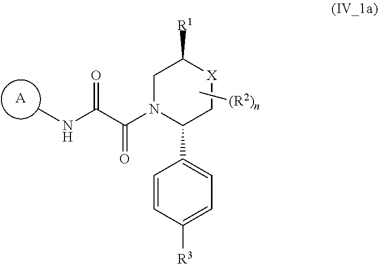

wherein X, Ring A, R¹, R² R³ and n are as defined herein.

In other embodiments, compounds of Formula (I) are of Formula (IV_2a),

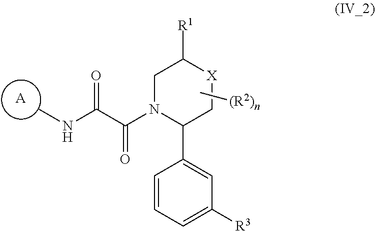

wherein X, Ring A, R¹, R² R³ and n are as defined herein.

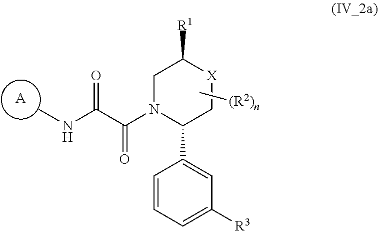

In certain embodiments, compounds of Formula (I) are of Formula (IV_3),

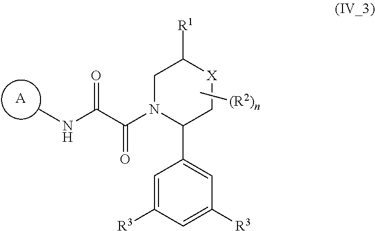

wherein X, Ring A, R¹, R² R³ and n are as defined herein.

In certain embodiments, compounds of Formula (I) are of Formula (IV_3a),

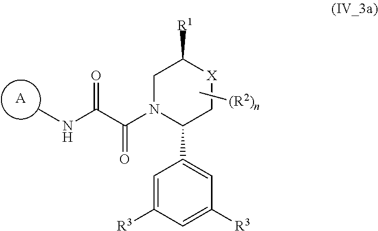

wherein X, Ring A, R¹, R² R³ and n are as defined herein.

In certain embodiments, compounds of Formula (I) are of Formula (IV_4a),

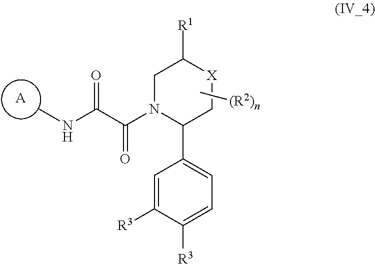

wherein X, Ring A, R¹, R² R³ and n are as defined herein.

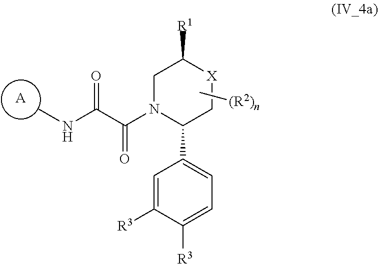

In certain embodiments, compounds of Formula (I) are of Formula (IV_5),

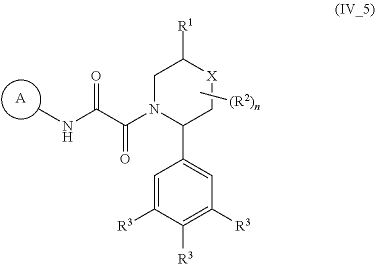

wherein X, Ring A, R¹, R² R³ and n are as defined herein.

In certain embodiments, compounds of Formula (I) are of Formula (IV_5a),

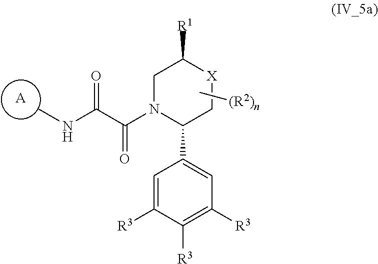

wherein X, Ring A, R¹, R² R³ and n are as defined herein.

In some embodiments, Ring B is phenyl substituted with halo (e.g., fluoro, chloro, bromo), —C₁-C₆ alkyl (e.g., -Me), —C₁-C₆ haloalkyl (e.g., —CF₃), —C₁-C₆ heteroalkoxy (e.g., —OCH₂CH₂N(CH₃)₂), or 3-10 member heterocyclyl (e.g., piperazinyl (e.g., N-Me piperazinyl)). In some embodiments, Ring B is phenyl substituted with —F, —Cl, -Me, —CF₃, —OCH₂CH₂N(CH₃)₂) or N-Me piperazinyl. In some embodiments, Ring B is phenyl substituted with halo (e.g., —F, —Cl, —Br). In some embodiments, Ring B is phenyl substituted with -Me.

In some embodiments, Ring B is phenyl substituted with —CF₃.

In some embodiments, Ring B is an optionally substituted 9-10 membered bicyclic aryl (e.g., naphthalenyl). In some embodiments, Ring B is naphthalenyl (e.g., naphthalen-1-yl, naphthalen-2-yl). In some embodiments, Ring B is naphthalen-2-yl. In some embodiments, Ring B is an optionally substituted bicyclic aryl containing a phenyl ring fused with a C₅-C₆ carbocycle (e.g., tetrahydronaphthyl, dihydroindenyl). In some embodiments, Ring B is 1,2,3,4-tetrahydronaphthalenyl. In some embodiments, Ring B is 2,3-dihydro-1H-indenyl. In some embodiments, Ring B is an optionally substituted bicyclic aryl containing a phenyl ring fused with a 5-6 membered heterocycle containing 1-3 heteroatoms independently selected from the group consisting of N, O and S or oxidized forms thereof (e.g., tetrahydronaphthalenyl, dihydroindenyl, 1,2,3,4 tetrahydroquinolinyl, 1,2 dihydroquinolinyl, 1,2-dihydroisoquinolinyl, tetrahydroisoquinolinyl, chromanyl, indolinyl, isoindolinyl, dihydrobenzoxazinyl, dihydrobenzofuranyl, benzodioxolyl, dihydrobenzimidazolyl).

In some embodiments, the bicyclic aryl is unsubstituted. In some embodiments, Ring B is unsubstituted naphthalenyl (e.g., naphthalen-1-yl, naphthalen-2-yl). In some embodiments, Ring B is unsubstituted naphthalen-2-yl. In some embodiments, the bicyclic aryl is substituted with 0, 1, 2 or 3 instances of R³, wherein each R³ is as defined in any of the embodiments described herein. In some embodiments, the bicyclic aryl is substituted with 1 instance of R³. In some embodiments, the bicyclic aryl is substituted with one instance of R³ wherein R³ is selected from the group consisting of halo (e.g., —F, —Cl, —Br), -Me, ═O.

In some embodiments, Ring B is an optionally substituted 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms).

In some embodiments, the 5-6 membered monocyclic heteroaryl is unsubstituted. In some embodiments, the 5-6 membered monocyclic heteroaryl is substituted with 0, 1, 2 or 3 instances of R³, wherein each R³ is as defined in any of the embodiments described herein. In some embodiments, the 5-6 membered monocyclic heteroaryl is substituted with 1 instance of R³. In some embodiments, the 5-6 membered monocyclic heteroaryl is substituted with 2 instances of R³. In some embodiments, the 5-6 membered monocyclic heteroaryl is substituted with 2 instances of R³. In some embodiments, the 5-6 membered monocyclic heteroaryl is substituted with 3 instances of R³.

In some embodiments, Ring B is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, Ring B is pyrazolyl (e.g., pyrazol-3-yl, pyrazol-4-yl, pyrazol-5-yl). In some embodiments, Ring B is pyrrolyl (e.g., pyrrol-1-yl, pyrrol-2-yl, pyrrol-3-yl). In some embodiments, Ring B is thiophenyl (e.g., thiophen-2-yl, thiophen-3-yl). In some embodiments, Ring B is furyl (e.g., fur-2-yl, fur-3-yl). In some embodiments, Ring B is thiazolyl (e.g., thiazol-2-yl, thiazol-4-yl, thiazol-5-yl). In some embodiments, Ring B is isothiazolyl (e.g., isothiazol-3-yl, isothiazol-4-yl, isothiazol-5-yl). In some embodiments, Ring B is oxazolyl (e.g., oxazol-2-yl, oxazol-4-yl, oxazol-5-yl). In some embodiments, Ring B is isoxazolyl (e.g., isoxazol-3-yl, isoxazol-4-yl, isoxazol-5-yl). In some embodiments, Ring B is imidazolyl (e.g., imidazol-2-yl, imidazol-4-yl). In some embodiments, Ring B is triazolyl. In some embodiments, Ring B is thiadiazolyl. In some embodiments, Ring B is oxadiazolyl. In certain embodiments, the 5-membered monocyclic heteroaryl is unsubstituted. In some embodiments, the 5-membered monocyclic heteroaryl is substituted with 1 instance of R. In some embodiments, the 5-membered monocyclic heteroaryl is substituted with 2 instances of R. In some embodiments, the 5-membered monocyclic heteroaryl is substituted with 3 instances of R.

In some embodiments, Ring B is a 6-membered monocyclic heteroaryl (e.g., pyridyl, pyrimidinyl, triazinyl, pyrazinyl, pyridazinyl). In some embodiments, the 6-membered monocyclic heteroaryl is unsubstituted. In some embodiments, the 6-membered monocyclic heteroaryl is substituted with 0, 1, 2 or 3 instances of R. In some embodiments, the 6-membered monocyclic heteroaryl is substituted with 1 instance of R³. In some embodiments, the 6-membered monocyclic heteroaryl is substituted with 2 instances of R³. In some embodiments, the 6-membered monocyclic heteroaryl is substituted with 3 instances of R. In some embodiments, Ring B is pyridinyl (e.g., pyridin-2-yl, pyridin-3-yl, pyridin-4-yl). In some embodiments, Ring B is pyridin-2-yl. In some embodiments, Ring B is pyridin-3-yl. In some embodiments, Ring B is pyridin-4-yl. In some embodiments, Ring B is pyrimidinyl (e.g, pyrimidin-2-yl, pyrimidin-4-yl, pyrimidin-5-yl).

In some embodiments, the compounds of Formula (I) are of Formula (V_1):

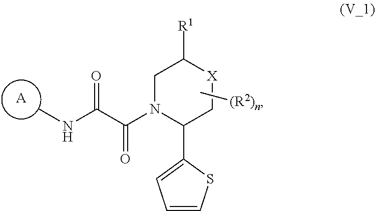

wherein X, Ring A, R¹, R² and n are as defined herein and the thiophenyl is substituted with 0, 1, 2 or 3 instances of R³ as defined herein. In some embodiments, the compounds of Formula (I) are of Formula (V_1a):

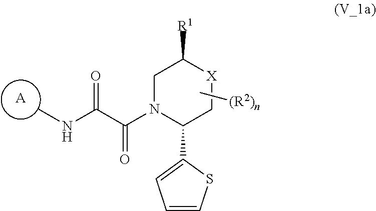

wherein X, Ring A, R¹, R² and n are as defined herein and the thiophenyl is substituted with 0, 1, 2 or 3 instances of R³ as defined herein. In some embodiments, the thiophenyl is unsubstituted. In some embodiments, the thiophenyl is substituted with one instance of W. In some embodiments, the thiophenyl is substituted with 2 instances of R. In some embodiments, the thiophenyl is substituted with 3 instances of R³.

In some embodiments, the compounds of Formula (I) are of Formula (V_2):

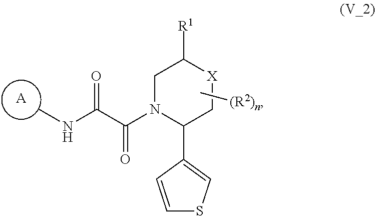

wherein X, Ring A, R¹, R² and n are as defined herein and the thiophenyl is substituted with 0, 1, 2 or 3 instances of R³ as defined herein. In some embodiments, the compounds of Formula (I) are of Formula (V_2a):

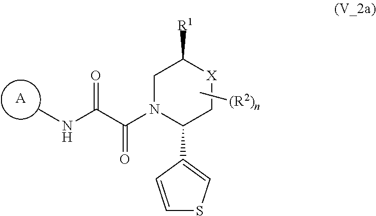

wherein X, Ring A, R¹, R² and n are as defined herein and the thiophenyl is substituted with 0, 1, 2 or 3 instances of R³ as defined herein. In some embodiments, the thiophenyl is unsubstituted. In some embodiments, the thiophenyl is substituted with one instance of R. In some embodiments, the thiophenyl is substituted with 2 instances of R³. In some embodiments, the thiophenyl is substituted with 3 instances of R.

In some embodiments, Ring B is an 8-10 membered bicyclic heteroaryl, wherein the bicyclic heteroaryl is optionally substituted (e.g., substituted with 0, 1, 2 or 3 instances of R³ wherein R³ is as defined herein). In certain embodiments, Ring B is an 8-10 membered bicyclic heteroaryl (e.g., a 5,5 bicyclic heteroaryl (e.g., 1H-thieno[2,3-c]pyrazolyl, 1H-thieno[3,2-c]pyrazolyl), a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl),1H-pyrazolo[3,4-b]pyridinyl, thiazolo[5,4-b]pyridinyl), or a 6, 6 bicyclic heteroaryl (e.g., quinolinyl, 1,5-naphthyridinyl, 1,2-dihydro-1,5-naphthyridinyl, 1,2,3,4-tetrahydro-1,8-naphthyridinyl, isoquinolinyl), wherein each bicyclic heteroaryl contains 1, 2, 3 or 4 heteroatoms independently selected from the group consisting of O, N and S, and wherein each bicyclic heteroaryl is optionally substituted (e.g., substituted with 0, 1, 2 or 3 instances of R³ wherein R³ is as defined herein). In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl),1H-pyrazolo[3,4-b]pyridinyl, thiazolo[5,4-b]pyridinyl) or a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl).

In some embodiments, Ring B is a 5,6 bicyclic heteroaryl (e.g., indolyl, benzofuranyl, 1H-indazolyl, 2H-indazolyl, benzo[b]thiophenyl, benzo[d]imidazolyl, benzo[d]thiazolyl, benzo[d]oxazolyl, [1,2,4]triazolo[4,3-a]pyridinyl, benzo[d]isothiazolyl, imidazo[1,2-a]pyridinyl, imidazo[1,2-a]pyridinyl, 1H-pyrazolo[4,3-b]pyridinyl),1H-pyrazolo[3,4-b]pyridinyl, thiazolo[5,4-b]pyridinyl).

In some embodiments, Ring B is a 6,6 bicyclic heteroaryl (e.g., quinolinyl, isoquinolinyl, 1,5-naphthyridinyl, 1,2-dihydro-1,5-naphthyridinyl, 1,2,3,4-tetrahydro-1,8-naphthyridinyl, isoquinolinyl)

In some embodiments, the bicyclic heteroaryl (e.g., the 5,5 bicyclic heteroaryl, 5,6 bicyclic heteroaryl, 6,6 bicyclic heteroaryl) contains 1, 2 or 3 heteroatoms selected from the group consisting of O, N and S. In some embodiments, the bicyclic heteroaryl contains 1 or 2 heteroatoms selected from the group consisting of O, N and S. In some embodiments, the bicyclic heteroaryl contains 1 heteroatom selected from the group consisting of O, N and S. In some embodiments, the bicyclic heteroaryl contains 2 heteroatoms selected from the group consisting of O, N and S. In some embodiments, the bicyclic heteroaryl contains 3 heteroatoms selected from the group consisting of O, N and S. In some embodiments, the bicyclic heteroaryl contains 4 heteroatoms selected from the group consisting of O, N and S.

In some embodiments Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl),1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted (e.g., substituted with 0, 1, 2 or 3 instances of R³).

In certain embodiments, Ring B is selected from the group consisting of 2H-indazolyl, quinolinyl, isoquinolinyl and benzo[d]thiazolyl, each optionally substituted (e.g., substituted with 0, 1, 2 or 3 instances of R³). In some embodiments, Ring B is optionally substituted 2H-indazolyl (e.g., substituted with 0, 1, 2 or 3 instances of R³). In some embodiments, Ring B is optionally substituted quinolinyl (e.g., substituted with 0, 1, 2 or 3 instances of R³). In some embodiments, Ring B is optionally substituted isoquinolinyl (e.g., substituted with 0, 1, 2 or 3 instances of R³). In some embodiments, Ring B is optionally substituted benzo[d]thiazolyl (e.g., substituted with 0, 1, 2 or 3 instances of R).

In some embodiments, Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted (e.g., substituted with 0, 1, 2 or 3 instances of R³).

In some embodiments, Ring B is independently selected from the group consisting of 2H-indazol-6-yl, 2H-indazol-5-yl, quinolin-6-yl, quinolin-7-yl, isoquinolin-6-yl and benzo[d]thiazol-5-yl, each optionally substituted (e.g., substituted with 0, 1, 2 or 3 instances of R³). In some embodiments, Ring B is optionally substituted 2H-indazol-6-yl (e.g., substituted with 0, 1, 2 or 3 instances of R³). In some embodiments, Ring B is optionally substituted 2H-indazol-5-yl (e.g., substituted with 0, 1, 2 or 3 instances of R³). In some embodiments, Ring B is optionally substituted quinolin-6-yl (e.g., substituted with 0, 1, 2 or 3 instances of R³). In some embodiments, Ring B is optionally substituted quinolin-7-yl (e.g., substituted with 0, 1, 2 or 3 instances of R³). In some embodiments, Ring B is optionally substituted isoquinolin-6-yl (e.g., substituted with 0, 1, 2 or 3 instances of R³). In some embodiments, Ring B is optionally substituted benzo[d]thiazol-5-yl (e.g., substituted with 0, 1, 2 or 3 instances of R³).

In some embodiments Ring B is an 8-10 membered bicyclic heteroaryl selected from the group consisting of

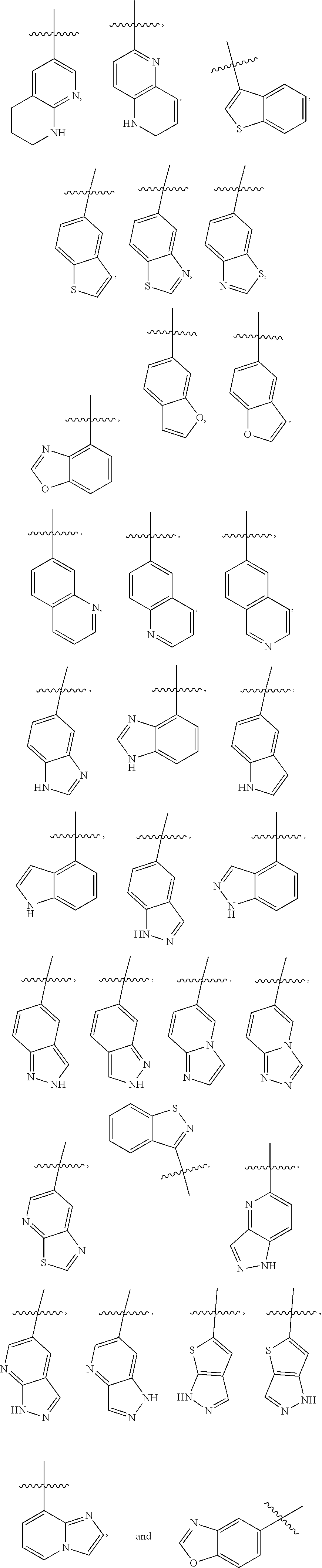

each optionally substituted (e.g., substituted with 0, 1, 2 or 3 instances of R³).

In certain embodiments, Ring B is selected from the group consisting of:

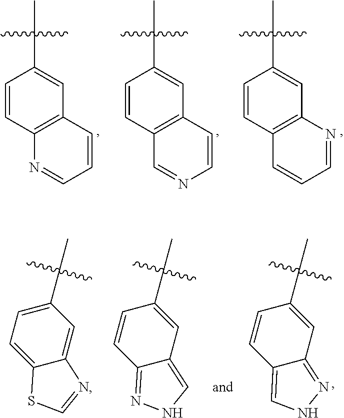

each optionally substituted (e.g., substituted with 0, 1, 2 or 3 instances of R³).

In some embodiments, the 8-10 membered bicyclic heteroaryl is unsubstituted. In some embodiments, the 8-10 membered bicyclic heteroaryl is substituted with 1 instance of R^{cc}. In some embodiments, the 8-10 membered bicyclic heteroaryl is substituted with 2 instances of R³. In some embodiments, 8-10 membered bicyclic heteroaryl is substituted with 3 instances of R³.

In certain embodiments, Ring B -is selected from the group consisting of:

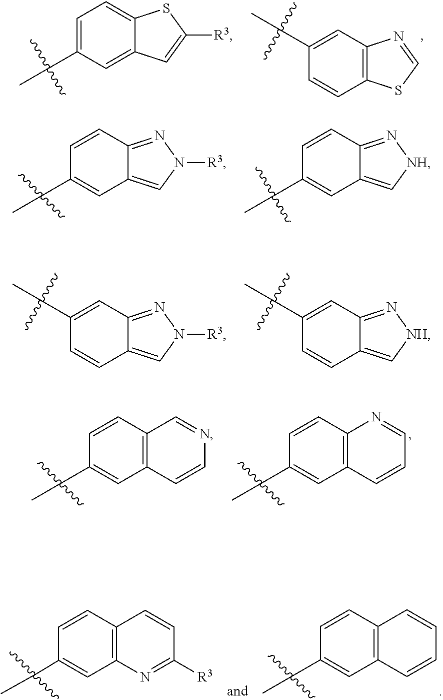

wherein R³ is as defined herein.

In certain embodiments, Ring B is selected from the group consisting of:

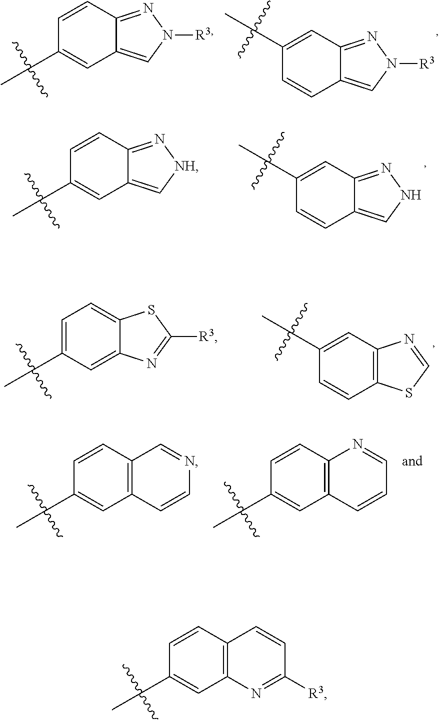

wherein R³ is as defined herein.

In some embodiments, Ring B is selected from the group consisting of:

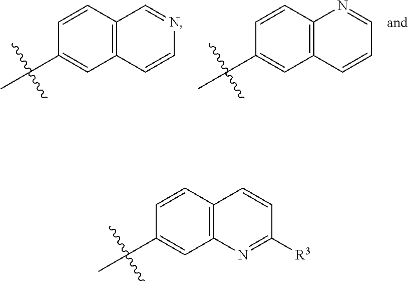

wherein R³ is as defined herein.

In some embodiments, Ring B is selected from the group consisting of:

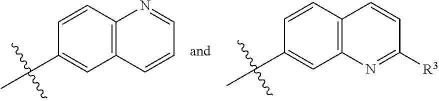

wherein R³ is as defined herein.

In certain embodiments, Ring B is selected from the group consisting of:

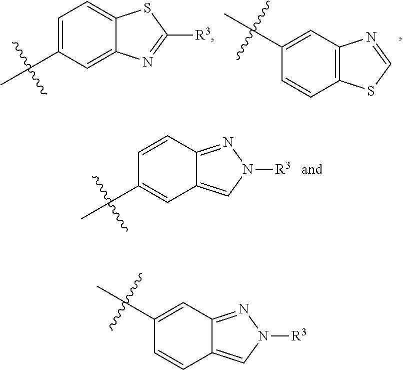

wherein R³ is as defined herein.

In some embodiments, Ring B is selected from the group consisting of:

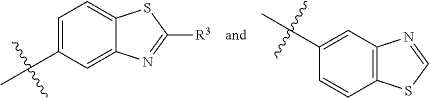

wherein R³ is as defined herein.

In certain embodiments, Ring B is

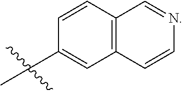 [SMILES: *c1ccc2cnccc2c1]

In certain embodiments, Ring B is

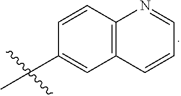 [SMILES: *c1ccc2ncccc2c1]

In certain embodiments, Ring B is

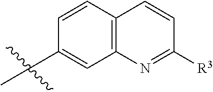

wherein R³ is as defined herein.

In certain embodiments, Ring B is

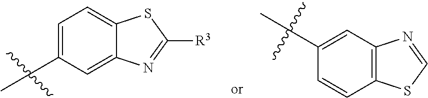

wherein R³ is as defined herein. In some embodiments, Ring B is

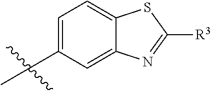

wherein R³ is as defined herein. In other embodiments, Ring B is

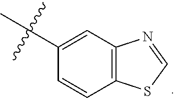 [SMILES: *c1ccc2scnc2c1]

In certain embodiments, Ring B is

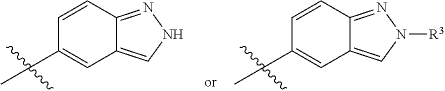

wherein R³ is as defined herein.

In certain embodiments, Ring B is

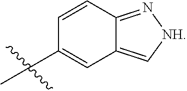 [SMILES: *c1ccc2n[nH]cc2c1]

In certain embodiments, Ring B is

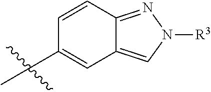

wherein R³ is as defined herein.

In certain embodiments, Ring B is

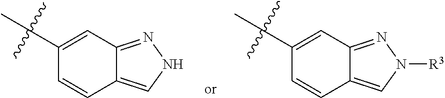

wherein R³ is as defined herein.

In certain embodiments, Ring B is

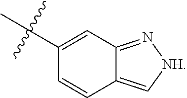 [SMILES: *c1ccc2c[nH]nc2c1]

In certain embodiments, Ring B is

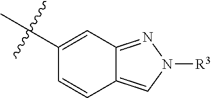

wherein R³ is as defined herein

In some embodiments, the compounds of Formula (I) are of Formula (VI):

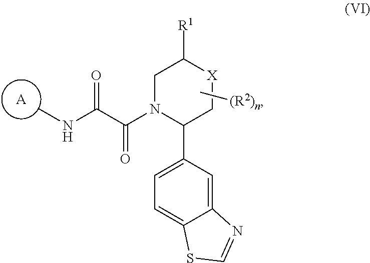

wherein X, Ring A, R¹, R² and n are as defined herein and the benzothiazole is substituted with 0, 1, 2 or 3 instances of R³ as defined herein. In some embodiments, the benzothiazole is unsubstituted. In some embodiments, the benzothiazole is substituted with one instance of R³. In some embodiments, the benzothiazole is substituted with 2 instances of R³. In some embodiments, the benzothiazole is substituted with 3 instances of R.

In some embodiments, the compounds of Formula (I) are of Formula (VIa):

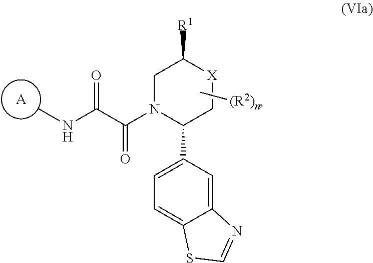

wherein X, Ring A, R¹, R² and n are as defined herein and the benzothiazole is substituted with 0, 1, 2 or 3 instances of R³ as defined herein.

In yet some embodiments, compounds of Formula (I) are of Formula (VI_1),

wherein X, Ring A, R¹, R² R³ and n are as defined herein.

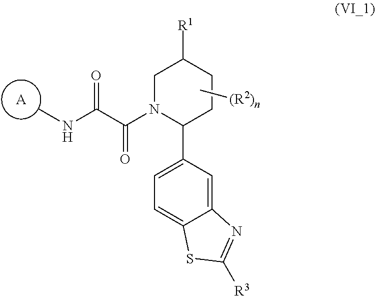

In some embodiments, compounds of Formula (I) are of Formula (VI_1a),

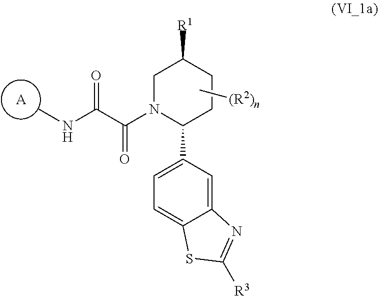

wherein X, Ring A, R¹, R² R³ and n are as defined herein.

As generally defined herein, each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1}and —S(═O)₂N(R^{a1})₂, wherein R^{a1} is as defined herein.

In some embodiments, each R¹ is independently selected from the group consisting of H, halo (e.g., —F, —Cl), —CN, —C₁-C₆ alkyl (e.g., -Me, -Et, —Pr, —ⁱPr, -sec-Bu, -^{t}Bu), 5-membered heteroaryl (e.g., pyrazolyl), —C₁-C₆ haloalkyl (e.g., —CF₃, —CHF₂, —CH₂CF₃), —C₁-C₆ hydroxyalkyl (e.g., —CH₂OH), —C₃-C₉ cycloalkyl (e.g., cyclopropyl, cyclobutyl, cyclopentyl, cyclohexyl), —OR^{a1} (e.g., —OH, —OCH₃, —OCHF₂), —N(R^{a1})₂ and —C(═O)N(R^{a1})₂ (e.g., —C(═O)NH₂, —C(═O)NHCH₃)), wherein each R^{a1} is as defined herein. In some embodiments, each R^{a1} is independently selected from the group consisting of H and —C₁-C₆ alkyl (e.g., -Me, -Et, —Pr, —ⁱPr, -^{tt}Bu, -^{t}Bu, -sec-Bu, -iso-Bu).

In certain embodiments, each R¹ is independently selected from the group consisting of H and methyl.

In some embodiments, R¹ is H. In some embodiments R¹ is -D.

In certain embodiments, R¹ is halo (e.g., fluoro, chloro, bromo, iodo). In some embodiments, R¹ is —Cl. In some embodiments, R¹ is —F. In some embodiments, R¹ is —Br. In some embodiments, R¹ is —I.

In some embodiments, R¹ is —CN.

In certain embodiments, R¹ is —C₁-C₆ alkyl. In some embodiments, R¹ is -Me. In some embodiments, R¹ is -Et. In some embodiments R¹ is —Pr or -iPr.

In some embodiments, R¹ is —C₁-C₆ hydroxyalkyl. In some embodiments, R¹ is hydroxymethyl (—CH₂OH).

In some embodiments, R¹ is —C₁-C₆ haloalkyl. In some embodiments, R¹ is trifluoromethyl (—CF₃). In other embodiments, R¹ is difluoromethyl (—CHF₂).

In some embodiments, R¹ is C₃-C₉ cycloalkyl (e.g., cyclopropyl, cyclobutyl, cyclopentyl, cyclohexyl). In some embodiments, R¹ is cyclopropyl. In some embodiments R¹ is cyclobutyl. In some embodiments, R¹ is cyclopentyl. In some embodiments, R¹ is cyclohexyl.

In some embodiments, R¹ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R¹ is oxetanyl. In some embodiments, R¹ is tetrahydropyranyl. In some embodiments, R¹ is tetrahydrofuranyl. In some embodiments, R¹ is azetidinyl. In some embodiments, R¹ is pyrrolidinyl. In some embodiments, R¹ is piperidinyl. In some embodiments, R¹ is piperazinyl. In some embodiments, R¹ is morpholinyl. In some embodiments, R¹ is azepanyl.

In some embodiments R¹ is cycloalkylalkyl (e.g., cyclopropylmethyl, cyclobutylmethyl, cyclopentylmethyl, cyclohexylmethyl, cycloheptylmethyl).

In some embodiments, R¹ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl).

In some embodiments, R¹ is arylalkyl. In some embodiments, R¹ is benzyl.

In some embodiments, R¹ is heteroarylalkyl (e.g., pyridinylmethyl, thiazolylmethyl, triazolylmethyl, pyrazolylmethyl).

In some embodiments, R¹ is —OR^{a1} wherein R^{a1} is as defined in any of the embodiments described herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy). In some embodiments, R¹ is hydroxy. In some embodiments, R¹ is methoxy. In some embodiments, R¹ is ethoxy. In some embodiments, R¹ is propoxy. In some embodiments, R¹ is isopropoxy. In some embodiments R¹ is difluoromethoxy. (—OCHF₂). In some embodiments, R¹ is trifluoromethoxy (—OCF₃).

In some embodiments, R¹ is —N(R^{a1})₂ wherein R^{a1} is as defined in any of the embodiments described herein (e.g., —NH₂, —NHR^{a1}, —N(CH₃)R^{a1}). In some embodiments, R¹ is —NH₂. In some embodiments, R¹ is —NHR^{a1} (e.g., —NHCH₃, —NHCH₂CH₃, —NHPr, —NHⁱPr, -NHcyclopropyl, -NHcyclobutyl). In some embodiments, R¹ is —N(CH₃)R^{a1} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R¹ is —C(═O)R^{a10}r —C(═O)OR^{a1} wherein R^{a1} is as defined in any of the embodiments described herein. In some embodiments, R¹ is —C(═O)R^{a1}wherein R^{a1} is as defined in any of the embodiments described herein. In some embodiments, R⁵ is —C(═O)alkyl. In some embodiments, R¹ is —C(═O)CH₃, —C(═O)cyclopropyl, —C(═O)cyclobutyl, —C(═O)^{t}Bu, —C(═O)ⁱPr, —C(═O)CH₂CH₂CH₃ or —C(═O)OCH₃. In some embodiments, R¹ is acetyl (—C(═O)CH₃). In some embodiments, R¹ is —C(═O)OR^{a1} wherein R^{a1} is as defined in any of the embodiments described herein. In some embodiments, R¹ is —COOH. In some embodiments, R¹ is COOCH₃.

In some embodiments, R¹ is —NR^{a1}C(═O)R^{a1}wherein R^{a1} is as defined in any of the embodiments described herein. In certain embodiments, R¹ is —NHC(═O)Ra (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)^{t}Bu, —NHC(═O)Cyclopropyl, —NHC(═O)Cyclobutyl). In some embodiments, R¹ is —N(CH₃)C(═O)R^{a1} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R¹ is —NR^{a1}C(═O)OR^{a1} wherein R^{a1} is as defined in any of the embodiments described herein. In certain embodiments, R¹ is —NHC(═O)OR^{a11} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R¹ is —N(CH₃)C(═O)OR^{a1} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R¹ is —C(═O)N(Ra)₂ wherein R^{a1} is as defined in any of the embodiments described herein (e.g., —C(═O)NH₂, —C(═O)NHR^{a1}, —C(═O)N(CH₃)R^{a1}). In some embodiments, R¹ is —C(═O)NH₂. In certain embodiments, R¹ is —C(═O)NHR^{a1} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R¹ is —C(═O)N(CH₃)R^{a1} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R¹ is —OC(═O)N(Ra)₂ wherein R^{a1} is as defined in any of the embodiments described herein. In certain embodiments, R¹ is —OC(═O)NHR^{a1} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R¹ is —OC(═O)N(CH₃)R^{a1} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R¹ is —S(═O)R^{a1}wherein R^{a1} is as defined in any of the embodiments described herein. In certain embodiments, R¹ is —S(═O)alkyl (e.g., —S(═O)CH₃, —S(═O)CH₂CH₃, —S(═O)CH₂CH₂CH₃, —S(═O)ⁱPr). In certain embodiments, R⁵ is —S(═O)cycloalkyl (e.g., —S(═O)cyclopropyl, —S(═O)cyclobutyl, —S(═O)cyclopentyl, —S(═O)cyclohexyl).

In some embodiments, R¹ is —S(═O)₂R^{a1}wherein R^{a1} is as defined in any of the embodiments described herein. In certain embodiments, R¹ is —S(═O)₂alkyl (e.g., —S(═O)₂CH₃, —S(═O)₂CH₂CH₃, —S(═O)₂Pr, —S(═O)₂′Pr). In certain embodiments, R¹ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R¹ is S(═O)₂aryl (e.g., —S(═O)₂phenyl).

In some embodiments, R¹ is -SR^{a1}wherein R^{a1} is as defined in any of the embodiments described herein. In certain embodiments, R¹ is -Salkyl (e.g., —SCH₃, —SCH₂CH₃, —SPr, —SⁱPr). In certain embodiments, R¹ is -Scycloalkyl (e.g., -Scyclopropyl, -Scyclobutyl, -Scyclopentyl, -Scyclohexyl). In certain embodiments, R¹ is -Saryl (e.g., -Sphenyl).

In some embodiments, R¹ is —S(═O)(═NR^{a1})R^{a1}wherein R^{a1} is as defined in any of the embodiments described herein. In certain embodiments, R¹ is —S(═O)(═NH)R^{a1} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R¹ is —S(═O)(═NCH₃)R^{a1} (e.g., —S(═O)(═NCH₃)CH, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R¹ is —NR^{a1}S(═O)₂R^{a1}wherein R^{a1} is as defined in any of the embodiments described herein. In certain embodiments, R¹ is —NHS(═O)₂alkyl (e.g., —NHS(═O)₂CH₃, —NHS(═O)₂CH₂CH₃, —NHS(═O)₂Pr, —NHS(═O)₂′Pr). In certain embodiments, R¹ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R¹ is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂′Pr). In certain embodiments, R¹ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R¹ is —S(═O)₂N(R^{a1})₂ wherein R^{a1} is as defined in any of the embodiments described herein. (e.g., —S(═O)₂NH₂, —S(═O)₂NHR^{a1}, —S(═O)₂N(CH₃)R^{a1}). In some embodiments, R¹ is —S(═O)₂NH₂. In some embodiments, R¹ is —S(═O)₂NHR^{a1} (e.g., —S(═O)₂NHCH₃, —S(═O)₂NHCH₂CH₃, —S(═O)₂NHPr, —S(═O)₂NH′Pr, —S(═O)₂NHcyclopropyl, —S(═O)₂NHcyclobutyl). In some embodiments, R¹ is —S(═O)₂N(CH₃)R^{a1} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R² is independently selected from the group consisting -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring), wherein R^{a2} is as defined herein.

In some embodiments of Formula (I), two R² groups are taken together with the atom to which they are attached to form a 3-10 membered spiro cycloalkyl (e.g., cyclopropyl, cyclobutyl, cyclopentyl, cyclohexyl, cycloheptyl) or spiro heterocyclyl ring (e.g., oxetanyl, tetrahydrofuranyl, tetrahydropyranyl, azetidinyl, pyrrolidinyl, piperidinyl, piperazinyl, morpholinyl, azepanyl, tetrahydrothiopyranyl, thiomorpholinyl).

In some embodiments of Formula (I), two R² groups are taken together with the adjacent atoms to which they are attached to form a 3-10 membered fused cycloalkyl (e.g., cyclopropyl, cyclobutyl, cyclopentyl, cyclohexyl, cycloheptyl) or fused heterocyclyl ring (e.g., oxetanyl, tetrahydrofuranyl, tetrahydropyranyl, azetidinyl, pyrrolidinyl, piperidinyl, piperazinyl, morpholinyl, azepanyl, tetrahydrothiopyranyl, thiomorpholinyl).

In some embodiments, the two R² groups taken together with the atoms to which they are attached form a bridged piperazine-containing or morpholine-containing heterocyclyl ring.

In other embodiments, the R² groups are not taken together to form cycloalkyl or heterocyclyl rings (i.e., each R² is independently selected from the group consisting of D, ═O, halo, —CN, —C₁-C₆ alkyl, —C₁-C₆ heteroalkyl, —C₁-C₆ haloalkyl, —C₃-C₉ cycloalkyl, 3-membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SW₂, —S(═O)(═NR^{a2})R^{a2}, —NR^{a2}S(═O)₂R^{a2} and —S(═O)₂N(R^{a2})₂, wherein R^{a2} is as defined herein.

In some embodiments, each R² is independently selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-6 membered heterocyclyl (e.g., oxetanyl, tetrahydrofuranyl), —OR^{a2}, —N(R^{a2})₂, —C(═O)R^{a2}, —C(═O)OR^{a2}, —NR^{a4}C(═O)R^{a2}, —NR^{a2}C(═O)OR^{a2}, —C(═O)N(R^{a2})₂, —OC(═O)N(R^{a2})₂, wherein each R^{a2} is as defined herein. In some embodiments, each R² is independently selected from the group consisting of halo, —CN, —C₁-C₆ alkyl, —C₁-C₆ haloalkyl, —C₃-C₉ cycloalkyl (e.g., cyclopropyl), 3-6 membered heterocyclyl (e.g., oxetanyl, tetrahydrofuranyl), —OR^{a2}, —N(R^{a2})₂,—C(═O)R^{a2}, —C(═O)N(OR^{a2})(R^{a2}), and —C(═O)N(R^{a2})₂, wherein each R^{a2} is as defined herein. In some embodiments, each R^{a2} is independently selected from the group consisting of H and —C₁-C₆ alkyl (e.g., -Me, -Et, —Pr, —ⁱPr, -^{tt}Bu, -^{t}Bu, -sec-Bu, -iso-Bu).

In some embodiments, each R² is independently selected from the group consisting of halo (e.g., —Cl), —C₁-C₆ alkyl (e.g., -Me, -Et, —Pr, —ⁱPr, -^{tt}Bu, -Bu, -sec-Bu, -iso-Bu), —C₁-C₆ haloalkyl (e.g., —CF₃, —CHF₂),—C₃-C₉ cycloalkyl (e.g., cyclopropyl), —C₁-C₆ haloalkoxy, (e.g., —OCF₃, —OCHF₂), —OCH₃, —C(═O)H, —C(═O)NHOH, and —C(═O)NH₂.

In some embodiments, each R² is independently selected from the group consisting of halo (e.g., —Cl), —C₁-C₆ alkyl (e.g., -Me, -Et, —Pr, —ⁱPr, -^{tt}Bu, -Bu, -sec-Bu, -iso-Bu) and —OCH₃.

In some embodiments, R² is -D.

In certain embodiments, R² is ═O.

In certain embodiments, R² is halo (e.g., fluoro, chloro, bromo, iodo). In some embodiments, R² is —Cl. In some embodiments, R² is —F. In some embodiments, R² is —Br. In some embodiments, R² is —I.

In some embodiments, R² is —CN.

In certain embodiments, R² is —C₁-C₆ alkyl. In some embodiments, R² is -Me. In some embodiments, R² is -Et. In some embodiments R² is —Pr or -iPr.

In some embodiments, R² is —C₁-C₆ heteroalkyl. In some embodiments, R² is methoxymethyl (—CH₂OCH₃). In some embodiments, R² is hydroxymethyl (—CH₂OH). In some embodiments, R² is aminomethyl (e.g., —CH₂NH₂, —CH₂NHCH₃, —CH₂N(CH₃)₂.

In some embodiments, R² is —C₁-C₆ haloalkyl. In some embodiments, R² is trifluoromethyl (—CF₃).

In some embodiments, R² is C₃-C₉ cycloalkyl (e.g., cyclopropyl, cyclobutyl, cyclopentyl, cyclohexyl). In some embodiments, R² is cyclopropyl. In some embodiments R² is cyclobutyl. In some embodiments, R² is cyclopentyl. In some embodiments, R² is cyclohexyl.

In some embodiments, R² is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl). In some embodiments, R² is 3-6 membered heterocyclyl (e.g., oxetanyl, tetrahydropyranyl, tetrahydrofuranyl, azetidinyl, pyrrolidinyl, piperidinyl, piperazinyl, morpholinyl). In some embodiments, R² is oxetanyl. In some embodiments, R² is tetrahydropyranyl. In some embodiments, R² is tetrahydrofuranyl. In some embodiments, R² is azetidinyl. In some embodiments, R² is pyrrolidinyl. In some embodiments, R² is piperidinyl. In some embodiments, R² is piperazinyl. In some embodiments, R² is morpholinyl. In some embodiments, R² is azepanyl.

In some embodiments R² is cycloalkylalkyl (e.g., cyclopropylmethyl, cyclobutylmethyl, cyclopentylmethyl, cyclohexylmethyl, cycloheptylmethyl). In some embodiments, R² is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl). In some embodiments, R² is arylalkyl (e.g., benzyl). In some embodiments, R² is heteroarylalkyl (e.g., pyridinylmethyl, thiazolylmethyl, triazolylmethyl, pyrazolylmethyl).

In some embodiments, R² is —OR^{a2} wherein each R^{a2} is as defined herein (e.g., —OH, methoxy, isopropoxy, difluoromethoxy (—OCHF₂), trifluoromethoxy (—OCF₃), ethoxy, propoxy, isopropoxy, cyclopropyloxy, cyclobutyloxy). In some embodiments R² is —OH. In some embodiments, R² is methoxy. In some embodiments, R² is ethoxy. In some embodiments, R² is propoxy. In some embodiments, R² is isopropoxy. In some embodiments R² is —C₁-C₆ haloalkoxy. In some embodiments R² is difluoromethoxy. (—OCHF₂). In some embodiments, R² is trifluoromethoxy (—OCF₃).

In some embodiments, R² is —N(R^{a2})₂ wherein each R^{a2} is as defined herein (e.g., —NH₂, —NHR^{a2}, —N(CH₃)R^{a2}). In some embodiments, R² is —NH₂. In some embodiments, R² is —NHR^{a2} (e.g., —NHCH₃, —NHCH₂CH₃, —NHPr, —NHⁱPr, -NHcyclopropyl, -NHcyclobutyl). In some embodiments, R² is —N(CH₃)R^{a2} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R² is —C(═O)R^{a2} wherein R^{a2} is as defined in any of the embodiments described herein. In some embodiments R² is —C(═O)R^{a2} wherein R^{a2} is C₁-C₆ alkyl, C₃-C₉ cycloalkyl or 3-10 membered heterocyclyl (e.g., —C(═O)CH₃, —C(═O)CH₂CH₃, —C(═O)^{t}Bu, —C(═O)ⁱPr, —C(═O)CH₂CH₂CH₃, —C(═O)^{t}Bu, —C(═O)cyclopropyl, —C(═O)cyclobutyl, —C(═O)oxetanyl, —C(═O)tetrahydropyranyl). In some embodiments, R² is —C(═O)CH₃, —C(═O)CH₂CH₃, —C(═O)^{t}Bu, —C(═O)ⁱPr, or —C(═O)CH₂CH₂CH₃. In some embodiments, R² is —C(═O)ⁱPr.

In some embodiments, R² is —C(═O)OR^{a2} wherein R^{a2} is as defined in any of the embodiments described herein. In some embodiments R² is —C(═O)OR^{a2} wherein R^{a2} is C₁-C₆ alkyl (e.g., —C(═O)OCH₃, —C(═O)OCH₂CH₃, —C(═O)O^{t}Bu, —C(═O)OⁱPr, —C(═O)OCH₂CH₂CH₃).

In some embodiments, R² is —NR^{a2}C(═O)R^{a2} wherein R^{a2} is as defined in any of the embodiments described herein. In certain embodiments, R² is —NHC(═O)R^{a2} (e.g., —NHC(═O)CH₃, —NHC(═O)CH₂CH₃, —NHC(═O)CH₂CH₂CH₃, —NHC(═O)ⁱPr, —NHC(═O)Bu, —NHC(═O)^{t}Bu, —NHC(═O)Cyclopropyl, —NHC(═O)Cyclobutyl). In certain embodiments, R² is NHC(═O)CH₃. In some embodiments, R² is —N(CH₃)C(═O)R^{a2} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R² is —NR^{a2}C(═O)OR^{a2} wherein R^{a2} is as defined in any of the embodiments described herein. In certain embodiments, R² is —NHC(═O)OR^{a2} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R² is —N(CH₃)C(═O)OR^{a2} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R² is —C(═O)N(R^{a2})₂ wherein R^{a2} is as defined in any of the embodiments described herein (e.g., —C(═O)NH₂, —C(═O)NHR^{a2}, —C(═O)N(CH₃)R^{a2}). In some embodiments, R² is —C(═O)NH₂. In certain embodiments, R² is —C(═O)NHR^{a2} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R² is —C(═O)N(CH₃)R^{a2} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R² is —OC(═O)N(R^{a2})₂ wherein R^{a2} is as defined in any of the embodiments described herein. In certain embodiments, R² is —OC(═O)NHR^{a2} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R² is —OC(═O)N(CH₃)R^{a2} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R² is —S(═O)R^{a2} wherein R^{a2} is as defined in any of the embodiments described herein. In certain embodiments, R² is —S(═O)alkyl (e.g., —S(═O)CH₃, —S(═O)CH₂CH₃, —S(═O)CH₂CH₂CH₃, —S(═O)ⁱPr). In certain embodiments, R² is —S(═O)cycloalkyl (e.g., —S(═O)cyclopropyl, —S(═O)cyclobutyl, —S(═O)cyclopentyl, —S(═O)cyclohexyl).

In some embodiments, R² is —S(═O)₂R^{a2} wherein R^{a2} is as defined in any of the embodiments described herein. In certain embodiments, R² is —S(═O)₂alkyl (e.g., —S(═O)₂CH₃, —S(═O)₂CH₂CH₃, —S(═O)₂Pr, —S(═O)₂′Pr). In certain embodiments, R² is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R² is S(═O)₂aryl (e.g., —S(═O)₂phenyl).

In some embodiments, R² is —SR^{a2} wherein R^{a2} is as defined in any of the embodiments described herein. In certain embodiments, R² is -Salkyl (e.g., —SCH₃, —SCH₂CH₃, —SPr, —SⁱPr). In certain embodiments, R² is -Scycloalkyl (e.g., -Scyclopropyl, -Scyclobutyl, -Scyclopentyl, -Scyclohexyl). In certain embodiments, R² is -Saryl (e.g., -Sphenyl).

In some embodiments, R² is —S(═O)(═NR^{a2})R^{a2} wherein R^{a2} is as defined in any of the embodiments described herein. In certain embodiments, R² is —S(═O)(═NH)R^{a2} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R² is —S(═O)(═NCH₃)R^{a2} (e.g., —S(═O)(═NCH₃)CH, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R² is —NR^{a2}S(═O)₂R^{a2} wherein R^{a2} is as defined in any of the embodiments described herein. In certain embodiments, R² is —NHS(═O)₂alkyl (e.g., —NHS(═O)₂CH₃, —NHS(═O)₂CH₂CH₃, —NHS(═O)₂Pr, —NHS(═O)₂′Pr). In certain embodiments, R² is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R² is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂′Pr). In certain embodiments, R² is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R² is —S(═O)₂N(R^{a2})₂ wherein R^{a2} is as defined in any of the embodiments described herein (e.g., —S(═O)₂NH₂, —S(═O)₂NHR^{a2}, —S(═O)₂N(CH₃)R^{a2}). In some embodiments, R² is —S(═O)₂NH₂. In some embodiments, R² is —S(═O)₂NHR^{a2} (e.g., —S(═O)₂NHCH₃, —S(═O)₂NHCH₂CH₃, —S(═O)₂NHPr, —S(═O)₂NH′Pr, —S(═O)₂NHcyclopropyl, —S(═O)₂NHcyclobutyl). In some embodiments, R² is —S(═O)₂N(CH₃)R^{a2} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein each alkyl, cycloalkyl, heterocyclyl, aryl, heteroaryl, cycloalkylalkyl, heterocyclylalkyl, arylalkyl and heteroarylalkyl of R³ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof) wherein R^{a3} is as defined herein.

In some embodiments, each alkyl, cycloalkyl, heterocyclyl, aryl, heteroaryl, cycloalkylalkyl, heterocyclylalkyl, arylalkyl and heteroarylalkyl of R³ is unsubstituted. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 1 instance of -Me, —OH, —C(═O)CH₃, —C(═O)NHCH₃, —NH₂ or —NHC(═O)CH₃. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 2 instances of -Me, —OH, —C(═O)CH₃, —C(═O)NHCH₃, —NH₂, —NHC(═O)CH₃ or a combination thereof. In some embodiments, each alkyl, cycloalkyl, heterocyclyl, aryl, heteroaryl, cycloalkylalkyl, heterocyclylalkyl, arylalkyl and heteroarylalkyl of R³ is independently substituted with 3 instances of -Me, —OH, —C(═O)CH₃, —C(═O)NHCH₃, —NH₂, —NHC(═O)CH₃ or a combination thereof.

In some embodiments, each R³ is independently selected from the group consisting of D, ═O, halo (e.g., —F, —Cl, —Br), —CN, —C₁-C₆ alkyl (e.g., -Me, -Et, —Pr, —ⁱPr, -sec-Bu, -^{t}Bu, —CH₂(CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂, —CH₂OH, —CH(OH)(CH₃),—C(OH)(CH₃)₂, —CH₂NH₂), —C₁-C₆ haloalkyl (e.g., —CHF₂, —CH₂CF₃, —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, piperidin-2-onyl, tetrahydropyridinyl (e.g., 1,2,3,6 tetrahydropyridinyl), piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-pyrrolidinyl, —CH(CH₃)CH₂-pyrrolidinyl, —(CH₂)₂-pyrrolidinyl, —(CH₂)₃-pyrrolidinyl, —CH₂-morpholinyl, —(CH₂)₂-morpholinyl), —OR^{a3} (e.g., —OH, —OCH₃, —OCH₂CH₃, —OCH₂CH₂N(CH₃)₂, —O-tetrahydrofuranyl, —O-tetrahydropyran-4-yl, —OCF₃, —OCHF₂,—OCH₂CH(CH₃)N(CH₃)₂, -Opiperidinyl, —O—(CH₂)₂-pyrrolidinyl, —O—CH₂-piperidinyl, —O—CH₂-oxetanyl, —O—CH₂-tetrahydrofuranyl, —O—CH₂-tetrahydropyranyl), —N(R^{a3})₂, (e.g., —NH₂, —NHR^{a3}, —NHCH₃, —N(CH₃)₂, —NHCH₂CF₃, —NH-oxetan-3-yl, —NH—(N-Me-2-oxo-pyrrolidin-3-yl) and —C(═O)N(R^{a3})₂, (e.g., —C(═O)NH₂, —C(═O)NHCH₃, —C(═O)CH₂CH₂N(CH₃)₂), wherein each alkyl, cycloalkyl, heterocyclyl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof) wherein R^{a3} is as defined herein.

In some embodiments, each R³ is independently selected from -D, ═O, halo (e.g., —F, —Cl, —Br), —CN, —C₁-C₆ alkyl (e.g., -Me, -Et, —Pr, —ⁱPr, -sec-Bu, -Bu), —C₁-C₆ heteroalkyl (e.g., —CH₂OH, —CH(OH)(CH₃),—C(OH)(CH₃)₂, —CH₂NH₂), —C₁-C₆ haloalkyl (e.g., —CHF₂, —CH₂CF₃, —CF₃), —C₃-C₉ cycloalkyl(e.g., cyclopropyl, cyclobutyl, cyclopentyl, cyclohexyl), 3-10 membered heterocyclyl (e.g., oxetanyl, pyrrolidinyl, piperidinyl, piperazinyl), 5-10 membered heteroaryl (e.g., pyrazolyl, thiazolyl, thiophenyl, pyridinyl), cycloalkylalkyl (e.g. —CH₂-cyclopropyl), heterocyclylalkyl (e.g., —CH₂-morpholinyl), heteroarylalkyl (e.g., —CH₂-triazolyl, —CH₂-imidazolyl, —CH₂-pyrazolyl), —OR^{a3} (e.g., —OH, -OCH₃, —O-tetrahydrofuranyl, —O-tetrahydropyran-4-yl, —OCF₃, —OCHF₂), —N(R^{a3})₂, (e.g., —NH₂, —NHR^{a3}, —NHCH₃, —N(CH₃)₂, —NHCH₂CF₃, —NH-oxetan-3-yl, —NH—(N-Me-2-oxo-pyrrolidin-3-yl), —NR^{a3}C(═O)R^{a3} (e.g., —NHC(═O)Me), —C(═O)N(R^{a3})₂, (e.g., —C(═O)NH₂, —C(═O)NHCH₃), —OC(═O)R^{a3} (e.g., —OC(═O)Me), —S(═O)R^{a3} (e.g., —SO₂Me), —NR^{a3}S(═O)₂R^{a3} (e.g., —NHSO₂Me) and —S(═O)₂N(R^{a3})₂ (e.g., —SO₂NH₂, —SO₂NHCH₃), wherein each alkyl, cycloalkyl, heterocyclyl, aryl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof), wherein R^{a3} is as defined herein.

In some embodiments, each R³ is independently selected from the group consisting of —CN, halo (e.g., —F, —Cl, —Br), —C₁-C₆ alkyl (e.g., -Me, -Et, —Pr, —ⁱPr, -sec-Bu, -—Bu, —CH₂(CH₃)(ⁱPr)), —C₁-C₆ heteroalkyl (e.g., —CH₂N(CH₃)₂, —CH(CH₃)CH₂N(CH₃)₂, —CH₂CH₂N(CH₃)₂, —CH₂C(CH₃)₂N(CH₃)₂, —CH₂CH₂CH₂N(CH₃)₂, —CH(CH₃)N(CH₃)₂, —CH₂CH(CH₃)N(CH₃)₂) —C₁-C₆ haloalkyl, (e.g., —CF₃), —C₃-C₉ cycloalkyl (e.g., cyclopropyl, cyclobutyl, cyclopentyl, cyclohexyl), 3-10 membered heterocyclyl (e.g., tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl), piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), cycloalkylalkyl (e.g., —CH₂-cyclopropyl), heterocyclylalkyl (e.g., —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OR^{a3} (e.g., —OH, —OCH₃, —OCH₂CH₃, —OCH₂CH₂N(CH₃)₂, —OCH₂CH(CH₃)N(CH₃)₂, —OCF₃, —OCHF₂, —O-piperidinyl, —OCH₂-pyrrolidinyl), —NR^{a3}C(═O)R^{a3} (e.g., —NHC(═O)CH₃), —NHC(═O)CH₂CH₂N(CH₃)₂), wherein each alkyl, cycloalkyl, heterocyclyl, cycloalkylalkyl, and heterocyclylalkyl is optionally substituted (e.g., with 0, 1, 2 or 3 instances of -Me, —OH, —C(═O)CH₃, —C(═O)NHCH₃, —NH₂, —NHC(═O)CH₃ or a combination thereof), wherein R^{a3} is as defined herein.

In some embodiments, R³ is independently selected from the group consisting of -Me, —CN, —F, —Cl, —Br, —CF₃, -Et, —Pr, —ⁱPr, -sec-Bu, -Bu, —CH₂(CH₃)(ⁱPr), —CH₂N(CH₃)₂, —CH(CH₃)CH₂N(CH₃)₂, —CH₂CH₂N(CH₃)₂, —CH₂CH₂CH₂N(CH₃)₂, —CH(CH₃)N(CH₃)₂, —CH₂CH(CH₃)N(CH₃)₂, —CH(CH₃)N(CH₃)₂, —CH₂C(CH₃)₂N(CH₃)₂, cyclopropyl, cyclobutyl, cyclopentyl, cyclohexyl, tetrahydrofuranyl, tetrahydropyranyl, oxetanyl, morpholinyl, pyrrolidinyl, piperidinyl, tetrahydropyridinyl (e.g., 1,2,3,6 tetrahydropyridinyl) piperidin-2-onyl, piperazinyl, piperazin-2-onyl, azetidinyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 1-azabicyclo[2.2.1]heptanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl, —CH₂-cyclopropyl, —CH₂-morpholinyl, —CH₂-piperidinyl, —CH₂-pyrrolidinyl, —CH(CH₃)CH₂-pyrrolidinyl, —(CH₂)₂-pyrrolidinyl, —(CH₂)₃-pyrrolidinyl), —OH, —OCH₃, —OCH₂CH₃, —OCH₂CH₂N(CH₃)₂, —OCF₃, —OCH₂CH(CH₃)N(CH₃)₂, —OCH₂(pyrrolidinyl), -Opiperidinyl, —OCHF₂, —C(═O)CH₃), and —C(═O)CH₂CH₂N(CH₃)₂), each optionally substituted (e.g., with 0, 1, 2 or 3 instances of -Me, —OH, —C(═O)CH₃, —C(═O)NHCH₃, —NH₂, —NHC(═O)CH₃ or a combination thereof).

In some embodiments, each R³ is independently selected from the group consisting of -D, ═O, —F, —Cl, —Br, —CN, -Me, -Et, —Pr, —ⁱPr, -sec-Bu, -Bu, —CHF₂, —CH₂CF₃, —CF₃, —CH₂OH, —CH(OH)(CH₃),—C(OH)(CH₃)₂, —CH₂NH₂, cyclopropyl, cyclobutyl, cyclopentyl, cyclohexyl, pyrrolidinyl, piperidinyl, piperazinyl, pyridinyl, pyrazolyl, thiazolyl, thiophenyl, —CH₂-cyclopropyl, —CH₂-morpholinyl, —CH₂₋₁,2,4-triazolyl, —CH₂-imidazolyl, —CH₂-pyrazolyl, —OH, —OCH₃, —OCF₃, —OCHF₂, —O-tetrahydrofuranyl, —O-tetrahydropyranyl, —O—(N-Me-2-oxo-pyrrolidinyl), —OCF₃, —OCHF₂,—NH₂, —NHCH₃, —NHCH₂CF₃, —NH-oxetanyl, —NH—(N-Me-2-oxo-pyrrolidinyl),—N(CH₃)₂, —NHC(═O)Me, —NHCH₂C(═O)N(Cl₃)₂, —NHCH(CH₃)C(═O)N(CH₃)₂, —C(═O)NH₂, —C(═O)NHCH₃, —OC(═O)Me, —SO₂Me, —NHSO₂Me,- SO₂NH₂ and —SO₂NHCH₃, wherein each cyclopropyl, cyclobutyl, cyclopentyl, cyclohexyl, pyrrolidinyl, piperidinyl, piperazinyl, pyridinyl, pyrazolyl, thiazolyl, thiophenyl, —CH₂-cyclopropyl, —CH₂-morpholin-4-yl, —CH₂₋₁,2,4-triazol-1-yl —CH₂-imidazol-1-yl and —CH₂-cyclopropyl, —CH₂-morpholinyl, —CH₂₋₁,2,4-triazolyl, —CH₂-imidazolyl and —CH₂-pyrazolyl can be independently substituted with 0, 1, 2 or 3 instances of -Me, —OH, —C(═O)CH₃, —C(═O)NHCH₃, —NH₂, —NHC(═O)CH₃ or a combination thereof.

In some embodiments, each R³ is independently selected from the group consisting of -D, ═O, —F, —Cl, —Br, —CN, -Me, -Et, —Pr, —ⁱPr, -sec-Bu, -Bu, —CHF₂, —CH₂CF₃, —CF₃, —CH₂OH, —CH(OH)(CH₃),—C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl,thiophen-2-yl, —CH₂-cyclopropyl, —CH₂-morpholin-4-yl, —CH₂₋₁,2,4-triazol-1-yl, —CH₂-imidazol-1-yl, —CH₂-pyrazol-1-yl, —OH, —OCH₃, —OCF₃, —OCHF₂, —O-tetrahydrofuran-3-yl, —O-tetrahydropyran-4-yl, —O—(N-Me-2-oxo-pyrrolidin-3-yl), —OCF₃, —OCHF₂,—NH₂, —NHCH₃, —NHCH₂CF₃, —NH-oxetan-3-yl, —NH—(N-Me-2-oxo-pyrrolidin-3-yl),—N(CH₃)₂, —NHC(═O)Me, —NHCH₂C(═O)N(CH₃)₂, —NHCH(CH₃)C(═O)N(CH₃)₂, —C(═O)NH₂, —C(═O)NHCH₃, —OC(═O)Me, —SO₂Me, —NHSO₂Me,- SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂₋₁,2,4-triazol-1-yl —CH₂-imidazol-1-yl and —CH₂-pyrazol-1-yl, can be independently substituted with 0, 1, 2 or 3 instances of -Me, —OH, —C(═O)CH₃, —C(═O)NHCH₃, —NH₂, —NHC(═O)CH₃ or a combination thereof.

In some embodiments, each R³ is independently selected from the group consisting of D, ═O, —F, —Cl, -Me, —ⁱPr, —CHF₂, —CF₃, cyclopropyl, piperidin-4-yl, piperazin-4-yl, pyrazol-1-yl, pyrazol-3-yl, pyrazol-4-yl, pyrazol-5-yl, —OH, —OCH₃, —OCF₃, —OCHF₂, wherein each cyclopropyl, piperazin-4-yl, pyrazol-1-yl, pyrazol-3-yl, pyrazol-4-yl and pyrazol-5-yl, can be independently substituted with 0, 1, 2 or 3 instances of -Me, —OH, —C(═O)CH₃, —C(═O)NHCH₃, —NH₂, —NHC(═O)CH₃ or a combination thereof.

In some embodiments, each R³ is independently selected from the group consisting of F, —Cl, -Me, —CF₃, N-Methylpiperazin-4-yl, N-methylpiperidin-4-yl, and —OCH₂CH₂N(CH₃)₂.

In some embodiments, R³ is H. In some embodiments R³ is -D.

In certain embodiments, R³ is —C₁-C₆ alkyl. In some embodiments, R³ is -Me. In some embodiments, R³ is -Et. In some embodiments R³ is —Pr or —ⁱPr. In some embodiments R³ is -^{t}Bu or -sec-Bu.

In certain embodiments, R³ is halo (e.g., fluoro, chloro, bromo, iodo). In some embodiments, R³ is —F or —Cl. In some embodiments, R³ is —Cl. In some embodiments, R³ is —F. In some embodiments, R³ is —Br. In some embodiments, R³ is —I.

In some embodiments, R³ is —CN.

In some embodiments, R³ is —C₁-C₆ heteroalkyl. In some embodiments, R³ is methoxymethyl (—CH₂OCH₃). In some embodiments, R³ is hydroxymethyl (—CH₂OH). In some embodiments, R³ is —CH(OH)CH₃, —C(OH)(CH₃)₂. In some embodiments, R³ is aminomethyl (e.g., —CH₂NH₂, —CH₂NHCH₃,—CH₂NHCH₂CH₃—CH₂N(CH₃)₂,—CH(CH₃)(N(CH₃)₂), —CH(CH₃)CH₂(N(CH₃)₂), —CH₂CH₂N(CH₃)₂, —CH₂CH₂CH₂N(CH₃)₂,—CH₂CH₂N(Me)(oxetan-3-yl), —CH(CH₃)N(CH₃)₂, —CH₂C(CH₃)₂N(CH₃)₂, —CH₂CH(CH₃)(N(CH₃)₂). In some embodiments, the heteroalkyl is further substituted with═O (e.g., —CH₂NHC(═O)CH₃). In some embodiments, R³ is —C₁-C₆ haloalkyl. In some embodiments, R³ is trifluoromethyl (—CF₃). In other embodiments, R³ is difluoromethyl (—CHF₂). In some embodiments R³ is trifluoroethyl (—CH₂CF₃)

In some embodiments, R³ is C₃-C₉ cycloalkyl (e.g., cyclopropyl, cyclobutyl, cyclopentyl, cyclohexyl). In some embodiments, R³ is cyclopropyl. In some embodiments, the cyclopropyl is substituted with 1 instance of -Me, —OH, —C(═O)CH₃, —C(═O)NHCH₃, —NH₂, or NHC(═O)CH₃. In some embodiments, R³ is cycloprop-1-yl substituted at the 1 position with 1 instance of -Me, —OH, —C(═O)CH₃, —C(═O)NHCH₃, —NH₂ or —NHC(═O)CH₃. In some embodiments R³ is cyclobutyl. In some embodiments, R³ is cyclopentyl.

In some embodiments, R³ is 3-10 membered heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl, decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2 or 3 instances of -Me, —OH, —C(═O)CH₃, —C(═O)NHCH₃, —NH₂, —NHC(═O)CH₃ or a combination thereof.

In some embodiments, R³ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl) or 5-10 membered bicyclic heterocyclyl (decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl) each substituted with 0, 1, 2 or 3 instances of -Me, —OH, —C(═O)CH₃, —C(═O)NHCH₃, —NH₂, —NHC(═O)CH₃ or a combination thereof.

In some embodiments, R³ is 3-8 membered monocyclic heterocyclyl (e.g., oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl, piperazin-2-onyl), each substituted with 0, 1, 2 or 3 instances of -Me, —OH, —C(═O)CH₃, —C(═O)NHCH₃, —NH₂, —NHC(═O)CH₃ or a combination thereof. In some embodiments, R³ is selected from the group consisting of oxetanyl, tetrahydropyranyl, tetrahydrofuranyl, azetidinyl, pyrrolidinyl, piperidinyl, tetrahydropyridinyl (e.g., 1,2,3,6 tetrahydropyridinyl), piperazinyl, morpholinyl, azepanyl, piperidin-2-onyl and piperazin-2-onyl, each substituted with 0, 1, 2 or 3 instances of -Me, —OH, —C(═O)CH₃, —C(═O)NHCH₃, —NH₂, —NHC(═O)CH₃ or a combination thereof. In some embodiments, R³ is oxetanyl. In some embodiments, R³ is tetrahydropyranyl. In some embodiments, R³ is tetrahydrofuranyl. In some embodiments, R³ is azetidinyl. In some embodiments, R³ is pyrrolidinyl. In some embodiments, R³ is piperidinyl. In some embodiments, R³ is tetrahydropyridinyl (e.g., 1,2,3,6 tetrahydropyridinyl). In some embodiments, R³ is piperazinyl. In some embodiments, R³ is morpholinyl. In some embodiments, R³ is azepanyl. In some embodiments, R³ is piperidin-2-onyl. In some embodiments, R³ is piperazin-2-onyl.

In some embodiments, R³ is selected from the group consisting of azetidin-3-yl, tetrahydrofuran-3-yl, tetrahydropyran-4-yl, oxetan-3-yl, morpholin-2-yl, pyrrolidin-1-yl, pyrrolidin-3-yl, piperidin-4-yl, 1,2,3,6 tetrahydropyridin-4-yl, piperidin-3-yl, piperidin-2-one-4-yl, piperazin-4-yl and piperazin-2-on-5-yl. In some embodiments, R³ is selected from the group consisting of azetidin-3-yl, tetrahydropyran-4-yl, oxetan-3-yl, morpholin-2-yl, pyrrolidin-3-yl, piperidin-4-yl, piperidin-3-yl, 1,2,3,6 tetrahydropyridin-4-yl, piperidin-2-one-4-yl and piperazin-4-yl.

In some embodiments, R³ is tetrahydrofuran-3-yl. In some embodiments, R³ is tetrahydropyran-4-yl. In some embodiments, R³ is oxetan-3-yl. In some embodiments, R³ is morpholin-2-yl. In some embodiments, R³ is pyrrolidin-1-yl. In some embodiments, R³ is pyrrolidin-3-yl. In some embodiments, R³ is piperidin-4-yl. In some embodiments, R³ is piperidin-3-yl. In some embodiments, R³ is 1,2,3,6 tetrahydropyridin-4-yl. In some embodiments, R³ is piperidin-2-one-4-yl. In some embodiments, R³ is piperazin-4-yl (e.g., 1-methyl-piperazin-4-yl). In some embodiments, R³ is piperazin-2-on-5-yl. In some embodiments, R³ is azetidin-3-yl.

In some embodiments, R³ is a 5-10 membered bicyclic heterocyclyl (e.g., decahydro-1,6-naphthyridinyl, 2-azaspiro[3.3]heptanyl, 5-oxa-2,8-diazaspiro[3.5]nonanyl, 8-azabicyclo[3.2.1]octanyl, 2-azabicyclo[2.2.2]octanyl, 3-azabicyclo[3.2.0]heptanyl, 3-azabicyclo[3.1.1]heptanyl, 3-azabicyclo[3.1.0]hexanyl, 2-azabicyclo[2.1.1]hexanyl, 1-azabicyclo[2.2.1]heptanyl, 3-azabicyclo[3.2.0]heptanyl, bicyclo[1.1.1]pentanyl, octahydrocyclopenta[c]pyrrolyl, decahydro-1,6-naphthyridinyl, octahydro-1H-pyrrolo[3,4-c]pyridinyl, decahydro-2,7-naphthyridinyl), each substituted with 0, 1, 2 or 3 instances of -Me, —OH, —C(═O)CH₃, —C(═O)NHCH₃, —NH₂, —NHC(═O)CH₃ or a combination thereof.

In some embodiments, the heterocyclyl is substituted with 0, 1, 2 or 3 instances of -D, ═O, -Me, —CD3, -Et, —C(═O)CH₃ cyclopropyl, oxetan-3-yl, —OH, —N(CH₃)₂, —CH₂N(CH₃)₂ or —C(═O)NHCH₃. In some embodiments, the heterocyclyl is substituted with 0 or 1 instances of -Me.

In some embodiments, R³ is monocyclic heterocyclylalkyl (e.g., oxetanylmethyl, aziridinylmethyl, tetrahydrofuranylmethyl, pyrrolidinylmethyl, pyrrolidinylethyl, pyrrolidinylpropyl, —CH(CH₃)CH₂-pyrrolidinyl, tetrahydropyranylmethyl, piperidinylmethyl, piperazinylmethyl, morpholinylmethyl, azepanylmethyl). In some embodiments, R³ is selected from the group consisting of —CH₂-oxetan-3-yl, —CH₂-piperidin-4-yl, —CH₂-pyrrolidin-1-yl, —(CH₂)₂-pyrrolidin-1-yl, —CH(CH₃)CH₂-pyrrolidin-1-yl. In some embodiments, R³ is selected from the group consisting of —CH₂-piperidin-4-yl, —CH₂-pyrrolidin-1-yl, —(CH₂)₂-pyrrolidin-1-yl, —CH(CH₃)CH₂-pyrrolidin-1-yl. In some embodiments, R³ is selected from the group consisting of —CH₂-piperidin-4-yl, —CH₂-pyrrolidin-1-yl, —(CH₂)₂-pyrrolidin-1-yl, —CH(CH₃)CH₂-pyrrolidin-1-yl.

In some embodiments, the monocyclic heterocyclylalkyl is unsubstituted. In some embodiments, the monocyclic heterocyclylalkylis optionally substituted (e.g., substituted with 0, 1, 2 or 3 instances of -Me, —OH, —C(═O)Me, —NHC(═O)Me or a combination thereof). In some embodiments, the monocyclic heterocyclylalkyl is substituted with 0, 1 or 2 instances of -Me. In some embodiments R³ is cycloalkylalkyl (e.g., cyclopropylmethyl, cyclobutylmethyl, cyclopentylmethyl, cyclohexylmethyl, cycloheptylmethyl). In some embodiments, R³ is cyclopropylmethyl.

In some embodiments, R³ is a 5-10 membered heteroaryl (e.g., a 5-6 membered monocyclic heteroaryl or an 8-10 membered bicyclic heteroaryl containing 1-3 heteroatoms selected from the group consisting of N, O and S). In some embodiments, R³ is a 5-6 membered monocyclic heteroaryl (e.g., a 5-membered monocyclic heteroaryl containing 1-3 heteroatoms selected from the group consisting of O, N and S, a 6-membered monocyclic heteroaryl containing 1-3 N heteroatoms). In some embodiments, R³ is a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R³ is a 6-membered monocyclic heteroaryl (e.g., pyridyl, pyrimidinyl, triazinyl, pyrazinyl, pyridazinyl). In some embodiments, the heteroaryl is substituted with 0, 1, 2 or 3 instances of -Me, —OH, —C(═O)CH₃, —C(═O)NHCH₃, —NH₂, —NHC(═O)CH₃ or a combination thereof.

In some embodiments, R³ is a 6-10 membered mono or bicyclic aryl. In some embodiments, R³ is phenyl. In some embodiments, the phenyl is substituted with 0, 1, 2 or 3 instances of -Me, —OH, —C(═O)Me, —NHC(═O)Me or a combination thereof.

In some embodiments, R³ is arylalkyl. In some embodiments, R³ is benzyl.

In some embodiments, R³ is heteroarylalkyl (e.g., pyridinylmethyl, thiazolylmethyl, triazolylmethyl, pyrazolylmethyl).

In some embodiments, R³ is —OR^{a3} wherein R^{a3} is as defined herein (e.g., hydroxy (—OH), methoxy, difluoromethoxy (—OCHF₂), trifluoromethoxy (—OCF₃), ethoxy, propoxy, isopropoxy, cyclopropyloxy, cyclobutyloxy, —O(CH₂)₂N(CH₃)₂, —O-tetrahydrofuran-3-yl, —O-tetrahydropyran-4-yl, —O(N-methyl piperidin-4-yl), —O—(N-Me-2-oxo-pyrrolidin-3-yl), —OCH₂CH(CH₃)N(CH₃)₂, —OCH₂(N-Methylpyrrolidin-2-yl), —O(N-methyl piperidin-4-yl), -0—(CH₂)₂-pyrrolidin-2-yl, —O—CH₂-piperidin-4-yl, —O—CH₂-oxetan-3-yl). In some embodiments, R³ is hydroxy. In some embodiments, R³ is methoxy. In some embodiments, R³ is difluoromethoxy (—OCHF₂). In some embodiments, R³ is trifluoromethoxy (—OCF₃). In some embodiments, R³ is ethoxy. In some embodiments, R³ is propoxy. In some embodiments, R³ is isopropoxy. In some embodiments, R³ is cyclopropyloxy. In some embodiments, R³ is cyclobutyloxy. In some embodiments, R³ is —O(CH₂)₂N(CH₃)₂.

In some embodiments, R³ is hydroxy. In some embodiments, R³ is methoxy. In some embodiments, R³ is ethoxy. In some embodiments, R³ is propoxy. In some embodiments, R³ is isopropoxy. In some embodiments R³ is difluoromethoxy. (—OCHF₂). In some embodiments, R³ is trifluoromethoxy (—OCF₃). In some embodiments, R³ is —O(CH₂)₂N(CH₃)₂.

In SOME embodiments, R³ is —N(R^{a3})₂ wherein R^{a3} is as defined herein (e.g., —NH₂, —NHR^{a3}, —N(CH₃)R^{a3}). In some embodiments, R³ is —NH₂. In some embodiments, R³ is —NHR^{a3} (e.g., —NHCH₃, —NHCH₂CH₃, —NHPr, —NHCH₂CF₃, —NHⁱPr, -NHcyclopropyl, -NHcyclobutyl). In some embodiments, R³ is NHCH₂CF₃. In some embodiments, R³ is —N(CH₃)R^{a3} (e.g., —N(CH₃)₂, —N(CH₃)CH₂CH₃, —N(CH₃)CH₂CH₂CH₃, —N(CH₃)ⁱPr, —N(CH₃)cyclopropyl, —N(CH₃)cyclobutyl).

In some embodiments, R³ is —C(═O)R^{a3} or —C(═O)OR^{a3} wherein R^{a3} is as defined herein. In some embodiments, R³ is —C(═O)R^{a3} wherein R^{a3} is as defined in any of the embodiments described herein. In some embodiments, R³ is —C(═O)alkyl. In some embodiments, R³ is —C(═O)CH₃, —C(═O)cyclopropyl, —C(═O)cyclobutyl, —C(═O)^{t}Bu, —C(═O)ⁱPr, —C(═O)CH₂CH₂CH₃, —C(═O)OCH₃ or —C(═O)CH₂CH₂N(CH₃)₂. In some embodiments, R³ is acetyl (—C(═O)CH₃). In some embodiments, R³ is —C(═O)CH₂CH₂N(CH₃)₂. In some embodiments, R³ is —C(═O)OR^{a3}. In some embodiments, R³ is —COOH. In some embodiments, R³ is COOCH₃.

In some embodiments, R³ is —NR^{a3}C(═O)R^{a3} wherein R^{a3} is as defined herein. In certain embodiments, R³ is —NHC(═O)R^{a3} (e.g., —NHC(═O)CH, —NHC(═O)CH₂CH₃, —NHC(═O)CH₂CH₂CH₃, —NHC(═O)ⁱPr, —NHC(═O)Bu, —NHC(═O)^{t}Bu, —NHC(═O)Cyclopropyl, —NHC(═O)Cyclobutyl, —C(═O)CH₂CH₂N(CH₃)₂). In some embodiments, R³ is —NHC(═O)CH₃. In some embodiments, R³ is —C(═O)CH₂CH₂N(CH₃)₂.

In some embodiments, R³ is —N(CH₃)C(═O)R^{a3} (e.g., —N(CH₃)C(═O)CH₃, —N(CH₃)C(═O)CH₂CH₃, —N(CH₃)C(═O)CH₂CH₂CH₃, —N(CH₃)C(═O)ⁱPr, —N(CH₃)C(═O)Bu, —N(CH₃)C(═O)^{t}Bu, —N(CH₃)C(═O)Cyclopropyl, —N(CH₃)C(═O)Cyclobutyl).

In some embodiments, R³ is —NR^{a3}C(═O)OR^{a3} wherein R^{a3} is as defined herein. In certain embodiments, R³ is —NHC(═O)OR^{a3} (e.g., —NHC(═O)OCH₃, —NHC(═O)OCH₂CH₃, —NHC(═O)OCH₂CH₂CH₃, —NHC(═O)OⁱPr, —NHC(═O)OBu, —NHC(═O)O^{t}Bu, —NHC(═O)OCyclopropyl, —NHC(═O)OCyclobutyl). In some embodiments, R³ is —N(CH₃)C(═O)OR^{a3} (e.g., —N(CH₃)C(═O)OCH₃, —N(CH₃)C(═O)OCH₂CH₃, —N(CH₃)C(═O)OCH₂CH₂CH₃, —N(CH₃)C(═O)OⁱPr, —N(CH₃)C(═O)OBu, —N(CH₃)C(═O)O^{t}Bu, —N(CH₃)C(═O)OCyclopropyl, —N(CH₃)C(═O)OCyclobutyl).

In some embodiments, R³ is —C(═O)N(R^{a3})₂wherein R^{a3} is as defined herein (e.g., —C(═O)NH₂, —C(═O)NHR^{a3}, —C(═O)N(CH₃)R^{a3}). In some embodiments, R³ is —C(═O)NH₂. In certain embodiments, R³ is —C(═O)NHR^{a3} (e.g., —C(═O)NHCH₃, —C(═O)NHCH₂CH₃, —C(═O)NHPr, —C(═O)NHⁱPr, —C(═O)NHBu, —C(═O)NH^{t}Bu, —C(═O)NHCyclopropyl, —C(═O)NHCyclobutyl). In some embodiments, R³ is —C(═O)NHCH₃. In certain embodiments, R³ is —C(═O)N(CH₃)R^{a3} (e.g., —C(═O)N(CH₃)₂, —C(═O)N(CH₃)CH₂CH₃, —C(═O)N(CH₃)CH₂CH₂CH₃, —C(═O)N(CH₃)ⁱPr, —C(═O)N(CH₃)Bu, —C(═O)N(CH₃)^{t}Bu, —C(═O)N(CH₃)Cyclopropyl, —C(═O)N(CH₃)Cyclobutyl).

In some embodiments, R³ is —OC(═O)N(R^{a3})₂ wherein R^{a3} is as defined herein. In certain embodiments, R³ is —OC(═O)NHR^{a3} (e.g., —OC(═O)NHCH₃, —OC(═O)NHCH₂CH₃, —OC(═O)NHPr, —OC(═O)NHⁱPr, —OC(═O)NHBu, —OC(═O)NH^{t}Bu, —OC(═O)NHCyclopropyl, —OC(═O)NHCyclobutyl). In certain embodiments, R³ is —OC(═O)N(CH₃)R^{a3} (e.g., —OC(═O)N(CH₃)₂, —OC(═O)N(CH₃)CH₂CH₃, —OC(═O)N(CH₃)CH₂CH₂CH₃, —OC(═O)N(CH₃)ⁱPr, —OC(═O)N(CH₃)Bu, —OC(═O)N(CH₃)^{t}Bu, —OC(═O)N(CH₃)Cyclopropyl, —OC(═O)N(CH₃)Cyclobutyl).

In some embodiments, R³ is —OC(═O)R^{a3} wherein R^{a3} is as defined herein. (e.g., —OC(═O)CH₃, —OC(═O)CH₂CH₃, —OC(═O)CH₂CH₂CH₃, —OC(═O)CH₂CH₂CH₃, —OC(═O)Bu, —OC(═O)Bu, —OC(═O)Cyclopropyl, —OC(═O)Cyclobutyl). In some embodiments, R³ is —OC(═O)CH₃.

In some embodiments, R³ is —S(═O)R^{a3} wherein R^{a3} is as defined herein. In certain embodiments, R³ is —S(═O)alkyl (e.g., —S(═O)CH₃, —S(═O)CH₂CH₃, —S(═O)CH₂CH₂CH₃, —S(═O)ⁱPr). In some embodiments R³ is —S(═O)CH₃. In certain embodiments, R³ is —S(═O)cycloalkyl (e.g., —S(═O)cyclopropyl, —S(═O)cyclobutyl, —S(═O)cyclopentyl, —S(═O)cyclohexyl).

In some embodiments, R³ is —S(═O)₂R^{a3} wherein R^{a3} is as defined herein. In certain embodiments, R³ is —S(═O)₂alkyl (e.g., —S(═O)₂CH₃, —S(═O)₂CH₂CH, —S(═O)₂Pr, —S(═O)₂′Pr). In some embodiments R³ is —S(═O)₂CH₃. In certain embodiments, R³ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R³ is S(═O)₂aryl (e.g., —S(═O)₂phenyl).

In some embodiments, R³ is —SR^{a3} wherein R^{a3} is as defined herein. In certain embodiments, R³ is -Salkyl (e.g., —SCH₃, —SCH₂CH₃, —SPr, —SⁱPr). In certain embodiments, R³ is -Scycloalkyl (e.g., -Scyclopropyl, -Scyclobutyl, -Scyclopentyl, -Scyclohexyl). In certain embodiments, R³ is -Saryl (e.g., -Sphenyl).

In some embodiments, R³ is —S(═O)(═NR^{a3})R^{a3} wherein R^{a3} is as defined herein. In certain embodiments, R³ is —S(═O)(═NH)R^{a3} (e.g., —S(═O)(═NH)CH₃, —S(═O)(═NH)CH₂CH₃, —S(═O)(═NH)CH₂CH₂CH₃, —S(═O)(═NH)ⁱPr, —S(═O)(═NH)Bu, —S(═O)(═NH)^{t}Bu, —S(═O)(═NH)Cyclopropyl, —S(═O)(═NH)Cyclobutyl). In some embodiments, R³ is —S(═O)(═NCH₃)R^{a3} (e.g., —S(═O)(═NCH₃)CH₃, —S(═O)(═NCH₃)CH₂CH₃, —S(═O)(═NCH₃)CH₂CH₂CH₃, —S(═O)(═NCH₃)ⁱPr, —S(═O)(═NCH₃)Bu, —S(═O)(═NCH₃)^{t}Bu, —S(═O)(═NCH₃)Cyclopropyl, —S(═O)(═NCH₃)Cyclobutyl).

In some embodiments, R³ is —NR^{a3}S(═O)₂R^{a3} wherein R^{a3} is as defined herein. In certain embodiments, R³ is —NHS(═O)₂alkyl (e.g., —NHS(═O)₂CH₃, —NHS(═O)₂CH₂CH₃, —NHS(═O)₂Pr, —NHS(═O)₂′Pr). In certain embodiments, R³ is —NHS(═O)₂cycloalkyl (e.g., —NHS(═O)₂cyclopropyl, —NHS(═O)₂cyclobutyl, —NHS(═O)₂cyclopentyl, —NHS(═O)₂cyclohexyl). In certain embodiments, R³ is —N(CH₃)S(═O)₂alkyl (e.g., —N(CH₃)S(═O)₂CH₃, —N(CH₃)S(═O)₂CH₂CH₃, —N(CH₃)S(═O)₂Pr, —N(CH₃)S(═O)₂′Pr). In certain embodiments, R³ is —N(CH₃)S(═O)₂cycloalkyl (e.g., —N(CH₃)S(═O)₂cyclopropyl, —N(CH₃)S(═O)₂cyclobutyl, —N(CH₃)S(═O)₂cyclopentyl, —N(CH₃)S(═O)₂cyclohexyl).

In some embodiments, R³ is —S(═O)₂N(R^{a3})₂ wherein R^{a3} is as defined herein. (e.g., —S(═O)₂NH₂, —S(═O)₂NHR^{a3}, —S(═O)₂N(CH₃)R^{a3}). In some embodiments, R³ is —S(═O)₂NH₂. In some embodiments, R³ is —S(═O)₂NHR^{a3} (e.g., —S(═O)₂NHCH₃, —S(═O)₂NHCH₂CH₃, —S(═O)₂NHPr, —S(═O)₂NH′Pr, —S(═O)₂NHcyclopropyl, —S(=O)₂NHcyclobutyl). In some embodiments, R³ is —S(═O)₂N(CH₃)R^{a3} (e.g., —S(═O)₂N(CH₃)₂, —S(═O)₂N(CH₃)CH₂CH₃, —S(═O)₂N(CH₃)CH₂CH₂CH₃, —S(═O)₂N(CH₃)ⁱPr, —S(═O)₂N(CH₃)cyclopropyl, —S(═O)₂N(CH₃)cyclobutyl).

As generally defined herein, each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂wherein each alkyl, cycloalkyl, heterocyclyl, aryl, heteroaryl, cycloalkylalkyl, heterocyclylalkyl, arylalkyl and heteroarylalkyl is optionally substituted at any available position, wherein R^{a7} is as defined in any of the embodiments described herein.

In some embodiments, each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂, wherein each alkyl, cycloalkyl, heterocyclyl, aryl, heteroaryl, cycloalkylalkyl, heterocyclylalkyl, arylalkyl and heteroarylalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof, wherein R^{a7} is as defined in any of the embodiments described herein.

In some embodiments, each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, —C₆-C₁₀ aryl, 5-10 membered heteroaryl, arylalkyl, heteroarylalkyl, —C(═O)R^{a7} and —C(═O)OR^{a7}, wherein each alkyl, cycloalkyl, heterocyclyl, aryl, heteroaryl, arylalkyl and heteroarylalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) and wherein R^{a7} is as defined in any of the embodiments described herein.

In certain embodiments, R⁷ is selected from the group consisting of C₁-C₆ alkyl (e.g., -Me, -Et, —Pr, —ⁱPr, -^{tt}Bu, -^{t}Bu, -sec-Bu, -iso-Bu, neopentyl), —C(═O)R^{a7} and —C(═O)OR^{a7}, wherein the alkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂), wherein R^{a7} is as defined in any of the embodiments described herein.

In some embodiments, each R⁷ is independently selected from the group consisting of —C₁-C₆ alkyl (e.g., -Me, -Et, —Pr, —ⁱPr, -^{tt}Bu, -^{t}Bu, -sec-Bu, -iso-Bu, neopentyl), —C₃-C₉ cycloalkyl (e.g., cyclopropyl, cyclobutyl, cyclopentyl, cyclohexyl), —C(═O)R^{a7} and —C(═O)OR^{a7}, wherein the alkyl and cycloalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂).

In some embodiments, each R⁷ is independently selected from the group consisting of -Me, -Et, —Pr, —ⁱPr, -^{tt}Bu, -^{t}Bu, -sec-Bu, -iso-Bu, neopentyl, cyclopropyl, cyclobutyl, —C(═O)R^{a7} and —C(═O)OR^{a7}, wherein the cyclopropyl and cyclobutyl is substituted at any available position with 0, 1 or 2 instances of -Me.

In some embodiments, each R^{a7} is independently selected from the group consisting of H, —C₁-C₆ alkyl (e.g., -Me, -Et, —Pr, —ⁱPr, -^{tt}Bu, -Bu, -sec-Bu, -iso-Bu), C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl), 3-7 membered heterocyclyl (e.g., azetidinyl, oxetanyl, piperidyl, pyrrolidinyl, tetrahydrofuranyl, tetrahydropyranyl), cycloalkylalkyl (e.g., —CH₂-cyclopropyl, —CH₂-cyclobutyl, —CH₂-cyclopentyl, —CH₂-cyclohexyl, —CH₂-cycloheptyl), heterocyclylalkyl (e.g., —CH₂-azetidinyl, —CH₂-pyrrolidinyl, —CH₂-piperidinyl, —CH₂-tetrahydrofuranyl, —CH₂-tetrahydropyranyl), aryl (e.g., phenyl), 5-6 membered heteroaryl (e.g., pyridinyl, pyrimidinyl, pyrazinyl, thiophenyl, furyl, thiazolyl, imidazolyl, pyrazolyl), arylalkyl (e.g., benzyl) and heteroarylalkyl ((e.g., —CH₂-pydidinyl, —CH₂-pyrimidinyl, —CH₂-pyrazinyl, —CH₂-thiophenyl, —CH₂-furyl, —CH₂-thiazolyl, —CH₂-imidazolyl, —CH₂-pyrazolyl), wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is as defined in any of the embodiments described herein.

In some embodiments, each R^{a7} is independently selected from the group consisting of H, —C₁-C₆ alkyl (e.g., -Me, -Et, —Pr, —ⁱPr, -^{tt}Bu, -^{t}Bu, -sec-Bu, -iso-Bu), C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl), cycloalkylalkyl (e.g., —CH₂-cyclopropyl, —CH₂-cyclobutyl, —CH₂-cyclopentyl, —CH₂-cyclohexyl, —CH₂-cycloheptyl), wherein each alkyl, cycloalkyl, and cycloalkylalkyl is substituted at any available position with 0, 1, 2 or 3 instances of R⁵, wherein each R⁵ is as defined in any of the embodiments described herein.

In some embodiments, each R^{a7} is independently selected from the group consisting of —C₁-C₆ alkyl (e.g., -Me, -Et, —Pr, —ⁱPr, -^{tt}Bu, -Bu, -sec-Bu, -iso-Bu) substituted with 0, 1, 2 or 3 instances of R⁵, wherein each R is as defined in any of the embodiments described herein. In certain embodiments, each R⁵ is independently selected from the group consisting of halo (e.g., —F, —Cl), —CN, —C₁-C₆ haloalkyl (e.g., —CF₃, —CH₂CF₃, —CF₂CH₃, —CHF₂, —CH₂F) 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), and —OH.

In some embodiments, each R^{a7} is independently C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl) substituted at any available position with 0, 1, 2 or 3 instances of R⁵, wherein each R⁵ is as defined in any of the embodiments described herein. In some embodiments, R^{a7} is cyclopropyl substituted with 0, 1 or 2 instances of R⁵, wherein R is as defined in any of the embodiments described herein.

In some embodiments, each R^{a7} is independently —CH₂-cyclopropyl substituted at any available position with 0, 1, 2 or 3 instances of R⁵, wherein each R⁵ is as defined in any of the embodiments described herein.

In some embodiments, each R^{a7} is independently selected from the group consisting of -Me, -Et, —Pr, —ⁱPr, -^{tt}Bu, -^{t}Bu, -sec-Bu, -iso-Bu, cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl, —CH₂-cyclopropyl, —CH₂-cyclobutyl, —CH₂-cyclopentyl, —CH₂-cyclohexyl, each substituted at any available position with 0, 1, 2 or 3 instances of R⁵, wherein each R⁵ is as defined in any of the embodiments described herein.

In some embodiments each R^{a7} is independently selected from the group consisting of -Me, -Et, —ⁱPr, -Bu, -iso-Bu, cyclopropyl, —CH₂-cyclopropyl, each substituted at any available position with 0, 1, 2 or 3 instances of R⁵, wherein each R is as defined in any of the embodiments described herein.

In some embodiments of R^{a7}, R⁵ is selected from is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —R, —N(R^{b})₂, —C(═O)R, —C(═O)OR, —NR^{b}C(═O)R^{b}, —NR^{b}C(═O)OR, —C(═O)N(R^{b})₂, —OC(═O)N(R^{b})₂, —S(═O)R, —S(═O)₂R, —SR^{b}, —S(═O)(═NR^{b})R, —NR^{b}S(═O)₂R^{cc} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl)).

In some embodiments of R^{a7}, each R⁵ is independently selected from the group consisting of ═O, halo (e.g., —F, —Cl), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃).

In some embodiments of R^{a7}, each R⁵ is independently selected from the group consisting of halo (e.g., —F, —Cl), —CN, —C₁-C₆ haloalkyl (e.g., —CF₃, —CH₂CF₃, —CF₂CH₃, —CHF₂, —CH₂F) 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), and —OH.

In some embodiments of R^{a7}, each R⁵ is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F) and —OH.

In some embodiments, each R⁷ is independently selected from the group consisting

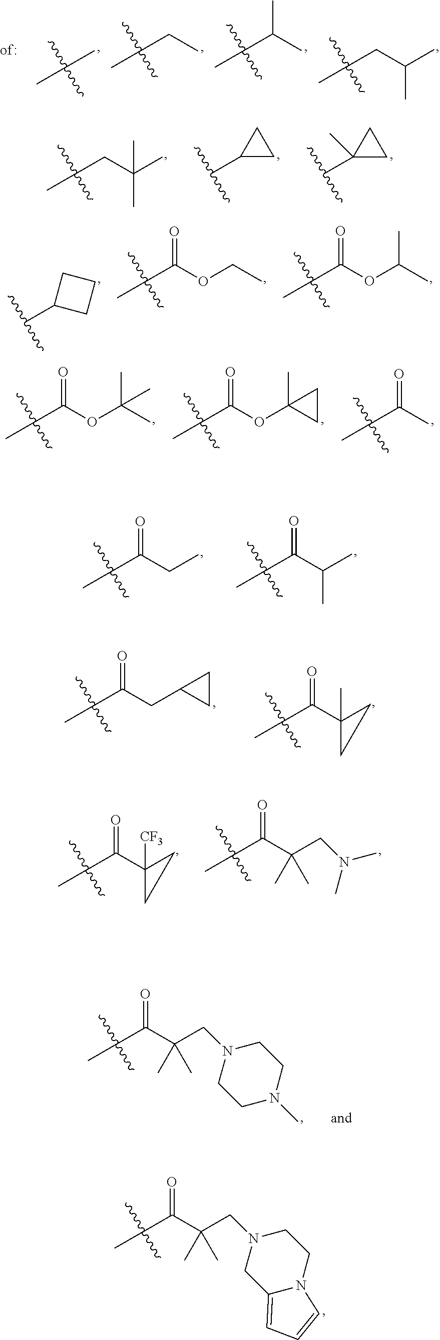

In some embodiments, each R⁷ is independently selected from the group consisting of:

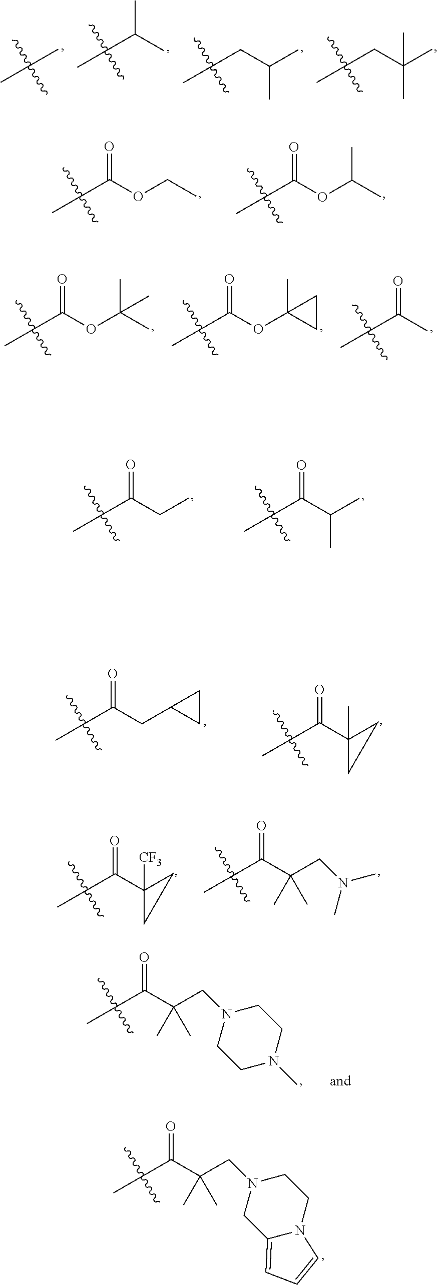

In some embodiments, each R^{a7} is independently selected from the group consisting of

In some embodiments, R⁷ is H. In some embodiments R⁷ is -D.

In certain embodiments, R⁷ is —C₁-C₆ alkyl (e.g., -Me, -Et, —Pr, —ⁱPr, -^{tt}Bu, -Bu, -sec-Bu, -iso-Bu, neopentyl), substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the alkyl is unsubstituted. In some embodiments, each R⁷ is independently selected from the group consisting of -Me, -Et, —ⁱPr, -iso-Bu, neopentyl. In some embodiments, R⁷ is selected from the group consisting of -Me, —ⁱPr, -iso-Bu and neopentyl. In some embodiments, R⁷ is -Me. In some embodiments, R¹ is -Et. In some embodiments R⁷ is -iPr. In some embodiments R⁷ is -iso-Bu. In some embodiments R⁷ is neopentyl.

In some embodiments, R⁷ is —C₁-C₆ heteroalkyl. In some embodiments, R⁷ is methoxymethyl (—CH₂OCH₃). In some embodiments, R⁷ is hydroxymethyl (—CH₂OH). In some embodiments, R⁷ is aminomethyl (e.g., —CH₂NH₂, —CH₂NHCH₃, —CH₂N(CH₃)₂.

In some embodiments, R⁷ is —C₁-C₆ haloalkyl. In some embodiments, R⁷ is trifluoromethyl (—CF₃). In other embodiments, R⁷ is difluoromethyl (—CHF₂).

In some embodiments, R⁷ is —C₃-C₉ cycloalkyl (e.g., cyclopropyl, cyclobutyl, cyclopentyl, cyclohexyl) substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the cycloalkyl is not substituted. In some embodiments, R⁷ is cyclopropyl. In some embodiments, R⁷ is

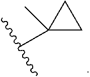 [SMILES: *C1(C)CC1]

In some embodiments R⁷ is cyclobutyl. In some embodiments, R⁷ is cyclopentyl. In some embodiments, R⁷ is cyclohexyl.

In some embodiments, R⁷ is 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl) substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the heterocyclyl is not substituted. In some embodiments, R⁷ is oxetanyl. In some embodiments, R⁷ is tetrahydropyranyl. In some embodiments, R⁷ is tetrahydrofuranyl. In some embodiments, R⁷ is azetidinyl. In some embodiments, R⁷ is pyrrolidinyl. In some embodiments, R⁷ is piperidinyl. In some embodiments, R⁷ is piperazinyl. In some embodiments, R⁷ is morpholinyl. In some embodiments, R⁷ is azepanyl.

In some embodiments R⁷ is cycloalkylalkyl (e.g., cyclopropylmethyl, cyclobutylmethyl, cyclopentylmethyl, cyclohexylmethyl, cycloheptylmethyl) substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the cycloalkyl alkyl is unsubstituted. In some embodiments, R⁷ is heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl) substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁—C₆ haloalkyl (e.g., —CF₃, —CHF₂) or a combination thereof. In some embodiments, the heterocyclylalkyl is not substituted.

In some embodiments, R⁷ is arylalkyl substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the arylalkyl is not substituted. In some embodiments, R⁷ is benzyl.

In some embodiments, R⁷ is heteroarylalkyl (e.g., pyridinylmethyl, thiazolylmethyl, triazolylmethyl, pyrazolylmethyl) substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof. In some embodiments, the heteroarylalkyl is not substituted.

In some embodiments, R⁷ is —C(═O)R^{a7} or —C(═O)OR^{a7} wherein R^{a7} is as defined in any of the embodiments described herein.

In some embodiments, R⁷ is —C(═O)R^{a7} wherein R^{a7} is as defined in any of the embodiments described herein. In some embodiments, R⁵ is —C(═O)alkyl, —C(═O)(C₃-C₉ cycloalkyl) or —C(═O)cycloalkylalkyl, wherein the alkyl, cycloalkyl and cycloalkylalkyl is substituted with 0, 1, 2 or 3 instances of R⁵ as defined in any of the embodiments described herein. In some embodiments, each R is independently selected from the group consisting of ═O, halo (e.g., —F, —Cl), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃).

In some embodiments, R⁷ is selected from the group consisting of —C(═O)CH₃, —C(═O)Et, —C(═O)^{t}Bu, —C(═O)ⁱPr, —C(═O)cyclopropyl, —C(═O)(1-methylcyclopropyl), —C(═O)(1-(trifluoromethyl)cyclopropyl), —C(═O)CH₂-cyclopropyl, —C(═O)(1-(dimethylamino)-2-methylpropan-2-yl), —C(═O)(2-methyl-1-(4-methylpiperazin-1-yl)propan-2-yl) and —C(═O)(1-(3,4-dihydropyrrolo[1,2-a]pyrazin-2(1H)-yl)-2-methylpropan-2-yl). In some embodiments, R⁷ is selected from the group consisting of —C(═O)CH₃, —C(═O)Et, —C(═O)^{t}Bu, —C(═O)ⁱPr, —C(═O)cyclopropyl, —C(═O)(1-methylcyclopropyl), —C(═O)(1-(trifluoromethyl)cyclopropyl) and —C(═O)CH₂-cyclopropyl. In some embodiments, R⁷ is —C(═O)CH₃. In some embodiments, R⁷ is —C(═O)Et. In some embodiments, R⁷ is —C(═O)^{t}Bu.

In some embodiments, R⁵ is —C(═O)ⁱPr. In some embodiments, R⁵ is —C(═O)cyclopropyl. In some embodiments, R⁷ is —C(═O)(1-methylcyclopropyl). In some embodiments, R⁷ is —C(═O)(1-(trifluoromethyl)cyclopropyl). In some embodiments, R⁵ is —C(═O)CH₂-cyclopropyl.

In some embodiments, R⁷ is —C(═O)OR^{a7} wherein R^{a7} is as defined in any of the embodiments described herein. In some embodiments, R⁷ is —C(═O)Oalkyl or —C(═O)O(C₃-C₉ cycloalkyl) wherein the alkyl and cycloalkyl are substituted with 0, 1, 2 or 3 instances of R as defined in any of the embodiments described herein. In some embodiments, each R⁵ is independently selected from the group consisting of ═O, halo (e.g., —F, —Cl), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃). In some embodiments, R⁷ is —C(═O)Oalkyl or —C(═O)O(C₃-C₉ cycloalkyl) wherein the cycloalkyl is substituted with 0 or 1 instances of —C₁-C₆ alkyl (e.g., -Me, -Et, iPr). In some embodiments, R⁷ is selected from —C(═O)OⁱPr, —C(═O)O^{t}Bu and —C(═O)O(1-methylcyclopropyl). In some embodiments, R⁷ is —C(═O)OⁱPr.

In some embodiments, R⁷ is —C(═O)O^{t}Bu. In some embodiments, R⁷ is —C(═O)O(1-methylcyclopropyl).

In some embodiments, R⁷ is —C(═O)N(R^{a7})₂ wherein R^{a7} is as defined in any of the embodiments described herein (e.g., —C(═O)NH₂, —C(═O)NHR^{a7}, —C(═O)N(CH₃)R^{a7}). In some embodiments, R⁷ is —C(═O)NH₂. In certain embodiments, R⁷ is —C(═O)NHR^{a7} (e.g., —C(═O)NHCH₃, —C(═O)NHEt, —C(═O)NHPr, —C(═O)NHⁱPr, —C(═O)NHBu, —C(═O)NH^{t}Bu, —C(═O)NHCyclopropyl, —C(═O)NHCyclobutyl). In certain embodiments, R⁷ is —C(═O)N(CH₃)R^{a7} (e.g., —C(═O)N(CH₃)₂, —C(═O)N(CH₃)Et, —C(═O)N(CH₃)Pr, —C(═O)N(CH₃)ⁱPr, —C(═O)N(CH₃)Bu, —C(═O)N(CH₃) #Bu, —C(═O)N(CH₃)Cyclopropyl, —C(═O)N(CH₃)Cyclobutyl).

In some embodiments, R⁷ is —S(═O)R^{a7} wherein R^{a7} is as defined in any of the embodiments described herein. In certain embodiments, R⁷ is —S(═O)alkyl (e.g., —S(═O)Me, —S(═O)Et, —S(═O)Pr, —S(═O)ⁱPr). In certain embodiments, R⁷ is —S(═O)cycloalkyl (e.g., —S(═O)cyclopropyl, —S(═O)cyclobutyl, —S(═O)cyclopentyl, —S(═O)cyclohexyl).

In some embodiments, R⁷ is —S(═O)₂R^{a7} wherein R^{a7} is as defined in any of the embodiments described herein. In certain embodiments, R⁷ is —S(═O)₂alkyl (e.g., —S(═O)₂Me, —S(═O)₂Et, —S(═O)₂Pr, —S(═O)₂′Pr). In certain embodiments, R⁷ is —S(═O)₂cycloalkyl (e.g., —S(═O)₂cyclopropyl, —S(═O)₂cyclobutyl, —S(═O)₂cyclopentyl, —S(═O)₂cyclohexyl). In some embodiments, R⁷ is S(═O)₂aryl (e.g., —S(═O)₂phenyl).

In some embodiments, R⁷ is —S(═O)₂N(R^{a7})₂ wherein R^{a7} is as defined in any of the embodiments described herein. (e.g., —S(═O)₂NH₂, —S(═O)₂NHR^{a7}, —S(═O)₂N(CH₃)R^{a7}). In some embodiments, R⁷ is —S(═O)₂NH₂. In some embodiments, R⁷ is —S(═O)₂NHR^{a7} (e.g., —S(═O)₂NHCH₃, —S(═O)₂NHEt, —S(═O)₂NHPr, —S(═O)₂NH′Pr, —S(═O)₂NHcyclopropyl, —S(═O)₂NHcyclobutyl). In some embodiments, R⁷ is —S(═O)₂N(CH₃)R^{a7} (e.g., —S(═O)₂N(CH₃)₂, —S(═O)₂N(CH₃)Et, —S(═O)₂N(CH₃)Pr, —S(═O)₂N(CH₃)ⁱPr, —S(═O)₂N(CH₃)cyclopropyl, —S(═O)₂N(CH₃)cyclobutyl).

As generally defined herein, each R^{a1}, R^{a2}, R^{a3}, R^{a4}, R^{a7}, R^{a8}, R^{a9}, R^{a10} and R^{a1} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R is as defined herein).

In some embodiments, each R^{a1}, R^{a2}, R^{a3}, R^{a4}, R^{a7}, R^{a8}, R^{a9}, R^{a10} and R^{a1} is independently unsubstituted. In some embodiments, each R^{a1}, R^{a2}, R^{a3}, R^{a4}, R^{a7}, R^{a8}, R^{a9}, R^{a10} and R^{a11} is independently substituted with 1 instance of R⁵. In some embodiments, each R^{a1}, R^{a2}, R^{a3}, R^{a4}, R^{a7}, R^{a8}, R^{a9}, R^{a10} and R^{a11} is independently substituted with 2 instances of R⁵.

In some embodiments, each R^{a1}, R^{a2}, R^{a3}, R^{a4}, R^{a7}, R^{a8}, R^{a9}, R^{a10} and R^{a1} is independently substituted with 3 instances of R⁵.

In some embodiments, each R^{a1}, R^{a2}, R^{a3}, R^{a4}, R^{a7}, R^{a8}, R^{a9}, R^{a10} and R^{a1} is independently selected from the group consisting of H, —C₁-C₆ alkyl, —C₁-C₆ haloalkyl, —C₁-C₆ heteroalkyl substituted with 0 or 1 instances of ═O, C₃-C₉ cycloalkyl substituted with 0 or 1 instances of ═O, -Me, —F, —Cl—, —CF₃, and 3-10 membered heterocyclyl substituted with 0 or 1 instances of ═O, -Me, —F, —Cl—, —CF₃ or a combination thereof.

In some embodiments, each R^{a1}, R^{a2}, R^{a3}, R^{a4}, R^{a7}, R^{a8}, R^{a9}, R^{a10} and R^{a11} is independently selected from the group consisting of H, —C₁-C₆ alkyl, (e.g., -Me, -Et, —Pr, —ⁱPr, -sec-Bu, -Bu), —C₁-C₆ haloalkyl (e.g., —CF₃, —CHF₂, —CH₂CF₃) and —C₁-C₆heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃)C(═O)N(CH₃)₂).

In some embodiments, each R^{a1}, R^{a2}, R^{a3}, R^{a4}, R^{a7}, R^{a8}, R^{a9}, R^{a10} and R^{a1} is independently selected from the group consisting of H, -Me, -Et, —Pr, —ⁱPr, -sec-Bu, -^{t}Bu, —CF₃, —CHF₂, —CH₂CF₃ and —CH₂CH₂N(CH₃)₂.

In some embodiments, each R^{a1}, R^{a2}, R^{a3}, R^{a4}, R^{a7}, R^{a8}, R^{a9}, R^{a10} and R^{a11} is independently selected from the group consisting of H, —C₁-C₆ alkyl (e.g., -Me, -Et, —Pr, —ⁱPr, -^{tt}Bu, -^{t}Bu, -sec-Bu, -iso-Bu) and —C₁-C₆ haloalkyl (e.g., —CHF₂, —CF₃). In some embodiments, each R^{a1}, R^{a2}, R^{a3}, R^{a4}, R^{a7}, R^{a8}, R^{a9}, R^{a1} and R^{a11} is independently selected from the group consisting of H and —C₁-C₆ alkyl (e.g., -Me, -Et, —Pr, —ⁱPr, -^{tt}Bu, -^{t}Bu, -sec-Bu, -iso-Bu).

In some embodiments, each R^{a1}, R^{a2}, R^{a3}, R^{a4}, R^{a7}, R^{a8}, R^{a9}, R^{a10} and R^{a11} is independently H.

In some embodiments, each R^{a1}, R^{a2}, R^{a3}, R^{a4}, R^{a7}, R^{a8}, R^{a9}, R^{a10} and R^{a11} is independently —C₁-C₆ alkyl (e.g., -Me, -Et, —Pr, —ⁱPr, -^{tt}Bu, -^{t}Bu, -sec-Bu, -iso-Bu). In some embodiments, each R^{a1}, R^{a2}, R^{a3}, R^{a4}, R^{a7}, R^{a8}, R^{a9}, R^{a10} and R^{a11} is independently -Me. In some embodiments, each R^{a1}, R^{a2}, R³, R^{a4}, R^{a7}, R^{a8}, R^{a9}, R^{a10} and R^{a11} is independently -Et.

In some embodiments, each R^{a1}, R^{a2}, R^{a3}, R^{a4}, R^{a7}, R^{a8}, R^{a9}, R^{a10} and R^{a11} is independently —Pr or —ⁱPr.

In some embodiments, each R^{a1}, R^{a2}, R^{a3}, R^{a4}, R^{a7}, R^{a8}, R^{a9}, R^{a10} and R^{a11} is independently —C₁-C₆ heteroalkyl. In some embodiments, each R^{a1}, R^{a2}, R^{a3}, R^{a4}, R^{a7}, R^{a8}, R^{a9} R^{a10} and R^{a11} is independently methoxymethyl (—CH₂OCH₃). In some embodiments, each R^{a1}, R^{a2}, R^{a3}, R^{a4}, R^{a7}, R^{a8}, R^{a9}, R^{a10} and R^{a11} is independently hydroxymethyl (—CH₂OH). In some embodiments, each R^{a1}, R^{a2}, R^{a3}, R^{a4}, R^{a7}, R^{a8}, R^{a9}, R^{a10} and R^{a11} is independently aminomethyl (e.g., —CH₂NH₂, —CH₂NHCH₃, —CH₂N(CH₃)₂, —CH₂CH₂N(CH₃)₂). In some embodiments, each R^{a1}, R^{a2}, R^{a3}, R^{a4}, R^{a7}, R^{a8}, R^{a9}, R^{a10} and R^{a11} is independently —CH₂CH₂N(CH₃)₂).

In some embodiments, each R^{a1}, R^{a2}, R^{a3}, R^{a4}, R^{a7}, R^{a8}, R^{a9}, R^{a10} and R^{a11} is independently —C₁-C₆ haloalkyl. In some embodiments, each R^{a1}, R^{a2}, R^{a3}, R^{a4}, R^{a7}, R^{a8}, R^{a9}, R^{a10} and R^{a11} is independently trifluoromethyl (—CF₃). In other embodiments, each R^{a1}, R^{a2}, R^{a3}, R^{a4}, R^{a7}, R^{a8}, R^{a9}, R^{a10} and R^{a11} is independently difluoromethyl (—CHF₂).

In some embodiments, each R^{a1}, R^{a2}, R^{a3}, R^{a4}, R^{a7}, R^{a8}, R^{a9}, R^{a10} and R^{a11} is independently C₃-C₉ cycloalkyl (e.g., cyclopropyl, cyclobutyl, cyclopentyl, cyclohexyl). In some embodiments, each R^{a1}, R^{a2}, R^{a3}, R^{a4}, R^{a7}, R^{a8}, R^{a9}, R^{a10} and R^{a11} is independently cyclopropyl. In some embodiments each R^{a1}, R^{a2}, R^{a3}, R^{a4}, R^{a7}, R^{a8}, R^{a9}, R^{a10} and R^{a11} is independently cyclobutyl. In some embodiments, each R^{a1}, R^{a2}, R^{a3}, R^{a4}, R^{a7}, R^{a8}, R^{a9}, R^{a10} and R^{a11} is independently cyclopentyl. In some embodiments, each R^{a1}, R^{a2}, R^{a3}, R^{a4}, R^{a7}, R^{a8} R^{a9}, R^{a10} and R^{a1} is independently cyclohexyl. In some embodiments, the cycloalkyl is substituted with 0, 1, 2 or 3 instances of R⁵, wherein each R⁵ is as defined herein.

In some embodiments, each R^{a1}, R^{a2}, R^{a3}, R^{a4}, R^{a7}, R^{a8}, R^{a9}, R^{a10} and R^{a1} is independently 3-10 membered heterocyclyl (e.g., oxetanyl, tetrahydropyranyl, tetrahydrofuranyl, azetidinyl, pyrrolidinyl, piperidinyl, piperazinyl, morpholinyl, azepanyl).

In some embodiments, each R^{a1}, R^{a2}, R^{a3}, R^{a4}, R^{a7}, R^{a8}, R^{a9}, R^{a10} and R^{a11} is independently is oxetanyl. In some embodiments, each R^{a1}, R^{a2}, R^{a3}, R^{a4}, R^{a7}, R^{a8}, R^{a9}, R^{a10} and R^{a1} is independently tetrahydropyranyl. In some embodiments, each R^{a1}, R^{a2}, R^{a3}, R^{a4}, R^{a7}, R^{a8}, R^{a9}, R^{a10} and R^{a11} is independently tetrahydrofuranyl. In some embodiments, each R^{a1}, R^{a2}, R^{a3}, R^{a4}, R^{a7}, R^{a8}, R^{a9}, R^{a10} and R^{a1} is independently azetidinyl. In some embodiments, each R^{a1}, R^{a2}, R^{a3}, R^{a4}, R^{a7}, R^{a8}, R^{a9}, R^{a10} and R^{a11} is independently pyrrolidinyl. In some embodiments, each R^{a1}, R^{a2}, R^{a3}, R^{a4}, R^{a7}, R^{a8}, R^{a9}, R^{a10} and R^{a11} is independently piperidinyl. In some embodiments, each R^{a1}, R^{a2}, R^{a3}, R^{a4}, R^{a7}, R^{a8}, R^{a9}, R^{a10} and R^{a11} is independently piperazinyl.

In some embodiments, each R^{a1}, R^{a2}, R^{a3}, R^{a4}, R^{a7}, R^{a8}, R^{a9}, R^{a10} and R^{a11} is independently morpholinyl. In some embodiments, each R^{a1}, R^{a2}, R^{a3}, R^{a4}, R^{a7}, R^{a8}, R^{a9}, R^{a10} and R^{a11} is independently azepanyl. In some embodiments, each heterocyclyl is substituted with 0, 1, 2 or 3 instances of R⁵, wherein each R⁵ is as defined herein.

In some embodiments, R^{a11}, R^{a2}, R^{a3}, R^{a4}, R^{a7}, R^{a8}, R^{a9}, R^{a10} and R^{a11} is independently a 5-10 membered heteroaryl (e.g., a 5-6 membered monocyclic heteroaryl or an 8-10 membered bicyclic heteroaryl containing 1-3 heteroatoms independently selected from the group consisting of N, O and S). In some embodiments, R^{a1}, R^{a2}, R^{a3}, R^{a4}, R^{a7}, R^{a8}, R^{a9}, R^{a10} and R^{a11} is independently a 5-6 membered monocyclic heteroaryl (e.g., a 5-membered monocyclic heteroaryl containing 1-3 heteroatoms independently selected from the group consisting of O, N and S, a 6-membered monocyclic heteroaryl containing 1-3 N heteroatoms). In some embodiments, R^{a1}, R^{a2}, R^{a3}, R^{a4}, R^{a7}, R^{a8}, R^{a9}, R^{a10} and R^{a1} is independently a 5-membered monocyclic heteroaryl (e.g., pyrazolyl, pyrrolyl, thiophenyl, furyl, thiazolyl, isothiazolyl, oxazolyl, isoxazolyl, imidazolyl, triazolyl, thiadiazolyl, oxadiazolyl). In some embodiments, R^{a11}, R^{a2}, R^{a3}, R^{a4}, R^{a7}, R^{a8}, R^{a9}, R^{a10} and R^{a1} is independently thiophenyl (e.g., thiophen-2-yl, thiophen-3-yl). In some embodiments, R^{a1}, R^{a2}, R^{a3}, R^{a4}, R^{a7}, R^{a8}, R^{a9}, R^{a10} and R^{a11} is independently pyrazolyl (e.g., pyrazol-1-yl, pyrazol-3-yl, pyrazol-5-yl). In some embodiments, R^{a11}, R^{a2}, R^{a3}, R^{a4}, R^{a7}, R^{a8}, R^{a9}, R^{a10} and R^{an} is independently thiazolyl (e.g., thiazol-2-yl, thiazol-4-yl, thiazol-5-yl). In some embodiments, R^{a1}, R^{a2}, R^{a3}, R^{a4}, R^{a7}, R^{a8}, R^{a9}, R^{a10} and R^{a11} is independently a 6-membered monocyclic heteroaryl (e.g., pyridyl, pyrimidinyl, triazinyl, pyrazinyl, pyridazinyl). In some embodiments, R^{a1}, R^{a2}, R^{a3}, R^{a4}, R^{a7}, R^{a8}, R^{a9}, R^{a1}and R^{a11} is independently pyridinyl (e.g., pyridin-2-yl, pyridin-3-yl, pyridin-4-yl). In some embodiments, R^{a1}, R^{a2}, R^{a3}, R^{a4}, R^{a7}, R^{a8}, R^{a9}, R^{a10} and R^{a11} is independently pyrimidinyl (e.g, pyrimidin-2-yl, pyrimidin-4-yl, pyrimidin-5-yl). In some embodiments, the heteroaryl is substituted with 0, 1, 2 or 3 instances of R⁵, wherein each R is as defined herein.

In some embodiments, R^{a1}, R^{a2}, R^{a3}, R^{a4}, R^{a7}, R^{a8}, R^{a9}, R^{a10} and R^{a11} is independently C₆-C₁₀ aryl. In some embodiments, R^{a1}, R^{a2}, R^{a3}, R^{a4}, R^{a7}, R^{a8}, R^{a9}, R^{a10} and R^{a11} is independently 6-10 membered mono or bicyclic aryl. In some embodiments, R^{a1}, R^{a2}, R^{a3}, R^{a4}, R^{a7}, R^{a8}, R^{a9}, R^{a10} and R^{a11} is independently phenyl. In some embodiments, the phenyl is substituted with 0, 1, 2 or 3 instances of R⁵ as defined herein. In some embodiments, the aryl is substituted with 0, 1, 2 or 3 instances of R⁵, wherein each R is as defined herein.

In some embodiments, each R^{a1}, R^{a2}, R^{a3}, R^{a4}, R^{a7}, R^{a8}, R^{a9}, R^{a10} and R^{a1} is independently cycloalkylalkyl (e.g., cyclopropylmethyl, cyclobutylmethyl, cyclopentylmethyl, cyclohexylmethyl, cycloheptylmethyl). In some embodiments, the cycloalkylalkyl is substituted with 0, 1, 2 or 3 instances of R⁵, wherein each R is as defined herein.

In some embodiments, each R^{a1}, R^{a2}, R^{a3}, R^{a4}, R^{a7}, R^{a8}, R^{a9}, R^{a10} and R^{a11} is independently heterocyclylalkyl (e.g., oxetanylmethyl, aziridinylmethyl, tetrahydrofuranylmethyl, pyrrolidinylmethyl, tetrahydropyranylmethyl, piperidinylmethyl, piperazinylmethyl, morpholinylmethyl, azepanylmethyl). In some embodiments, the heterocyclylalkyl is substituted with 0, 1, 2 or 3 instances of R⁵, wherein each R is as defined herein.

In some embodiments, each R^{a1}, R^{a2}, R^{a3}, R^{a4}, R^{a7}, R^{a8}, R^{a9}, R^{a10} and R^{a1} is independently arylalkyl. In some embodiments, each R^{a1}, R^{a2}, R^{a3}, R^{a4}, R^{a7}, R^{a8}, R^{a9}, R^{a10} and R^{an} is independently benzyl. In some embodiments, the arylalkyl is substituted with 0, 1, 2 or 3 instances of R⁵, wherein each R⁵ is as defined herein.

In some embodiments, each R^{a1}, R^{a2}, R^{a3}, R^{a4}, R^{a7}, R^{a8}, R^{a9}, R^{a10} and R^{a1} is independently heteroarylalkyl (e.g., pyridinylmethyl, thiazolylmethyl, triazolylmethyl, pyrazolylmethyl). In some embodiments, the heteroarylalkyl is substituted with 0, 1, 2 or 3 instances of R⁵, wherein each R⁵ is as defined herein.

As generally defined herein, each R⁵ is independently selected from the group consisting of ═O, halo, —CN, —C₁-C₆ alkyl, —C₁-C₆ heteroalkyl, —C₁-C₆ hydroxyalkyl, —C₁—C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR, —NR^{b}C(═O)R, —NR^{b}C(═O)OR, —C(═O)N(R^{b})₂, —OC(═O)N(R^{b})₂,—S(═O)R^{b}, —S(═O)₂R, —SR^{b}, —S(═O)(═NR^{b})R, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl). In some embodiments, each R^{b} is H or -Me.

In some embodiments, each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR, —NR^{b}C(═O)R^{b}, —NR^{b}C(═O)OR^{b}, —C(═O)N(R^{b})₂, —OC(═O)N(R^{b})₂, —S(═O)R^{b}, —S(═O)₂R, —SR^{b}, —S(═O)(═NR)R^{b}, —NR^{b}S(═O)₂R and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu)), and C₃-C₉ cycloalkyl (e.g., cyclopropyl, cyclobutyl, cyclopentyl, cyclohexyl). In some embodiments, each R^{b} is H or -Me.

In some embodiments, each R⁵ is independently selected from the group consisting of ═O, halo (e.g., —F, —Cl), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃).

In some embodiments, each R⁵ is independently selected from the group consisting ofhalo (e.g., —F, —Cl), —CN, —C₁-C₆ haloalkyl (e.g., —CF₃, —CH₂CF₃, —CF₂CH₃, —CHF₂, —CH₂F) 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), and —OH.

In some embodiments, each R⁵ is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F) and —OH

In some embodiments, each R⁵ is independently selected from the group consisting of F, —CN, -Me, -Et, —CH₂OH, —OH, —CF₃, —CH₂CF₃, —CF₂CH₃, —CHF₂, —CH₂F, —N(CH₃)₂, N-Me-piperazinyl and 1,2,3,4-tetrahydropyrrolo[1,2-a]pyrazin-2-yl.

In some embodiments, each R⁵ is independently selected from the group consisting of -Me, —CF₃, —N(CH₃)₂, N-Me-piperazinyl and 1,2,3,4-tetrahydropyrrolo[1,2-a]pyrazin-2-yl.

In some embodiments, each R⁵ is independently selected from the group consisting of F, —Cl—CN, -Me, -Et, iPr —CH₂OH, —CF₃, —CH₂CF₃, —CF₂CH₃, —CHF₂, —CH₂F, —OH, —OCH₃, —N(CH₃)₂, N-Me-piperazinyl and 1,2,3,4-tetrahydropyrrolo[1,2-a]pyrazin-2-yl.

In some embodiments, each R⁵ is independently selected from the group consisting of Me, —CF₃, —N(CH₃)₂, N-Me-piperazinyl and 1,2,3,4-tetrahydropyrrolo[1,2-a]pyrazin-2-yl.

In some embodiments, R is -Me. In some embodiments, R is —CF₃. In some embodiments, R⁵ is —N(CH₃)₂. In some embodiments, R⁵ is N-Me-piperazinyl. In some embodiments, R⁵ is 1,2,3,4-tetrahydropyrrolo[1,2-a]pyrazin-2-yl.

In one embodiment, provided is a compound selected from the group consisting of the compounds of Table 1, or pharmaceutically acceptable salts thereof.

Compounds described herein (e.g., a compound of Formula (I) to (1117a) or a compound of Table 1, or pharmaceutically acceptable salts thereof) are useful as inhibitors of PRMT5 (e.g., MTA uncompetitive PRMT5 inhibitors).

In one embodiment, provided is a compound selected from the compounds of Table 1, or pharmaceutically acceptable salts thereof.

Compounds described herein (e.g., a compound of Formula (I) or a compound of Table 1, or pharmaceutically acceptable salts thereof) are useful as inhibitors of PRMT5 (e.g., MTA uncompetitive PRMT5 inhibitors).

Table 1 indicates IC₅₀ and IC₉₀ values in an MTAP-isogenic cell line pair for exemplary compounds in an SDMA in-cell western assay (described in Example 119) (columns 4-6). HAP1 MTAP-intact is a cell line in which endogenous levels of MTAP are expressed, and HAP1 MTAP-deleted is an MTAP-null cell line. For Table 1, “a” and “aa” indicates an IC₅₀ of <5 nM, “b” and “bb” indicates an IC₅₀ equal to or greater than 5 nM but less than 50 nM, and “c” and “cc” indicates an IC₅₀ of greater than or equal to 50 nM in the HAP1 MTAP-intact (column 4) and the HAP1 MTAP-deleted (column 5) assays, respectively. Similarly, “aaa” indicates an IC₉₀ of <75 nM, “bbb” indicates an IC₉₀ equal to or greater than 75 nM but less than 125 nM, and “ccc” indicates an IC₉₀ of greater than or equal to 125 nM in the HAP1 MTAP-deleted (column 6) assay.

In column 7, “A” indicates an IC₅₀ ratio greater than or equal to 30 fold between the IC₅₀ in the HAP1 MTAP-intact cell line and the HAP1 MTAP-deleted cell line; “B” indicates an IC₅₀ ratio greater than or equal to 15 fold but lower than 30 fold between the IC₅₀ in the HAP1 MTAP-intact cell line and the HAP1 MTAP-deleted cell line; “C” indicates an IC₅₀ ratio of less than 15 fold between the IC₅₀ in the HAP1 MTAP-intact cell line and the HAP1 MTAP-deleted cell line. Compounds with a ratio in the SDMA in-cell western assay of equal to or greater than 3 fold are considered MTAP-selective.

Table 1 additionally indicates IC₅₀ values in a viability assay for the MTAP-deleted cell line (described in Example 120) (column 8), indicating the effect of treatment with compound on cell survival. In column 8, a value of A* indicates an IC₅₀ of less than 100 nM, a value of B* indicates an IC₅₀ equal to or greater than 100 nM but less than 1 μM, and a value of C* indicates an IC₅₀ greater than or equal to 1 μM.

Unless otherwise indicated, the absolute stereochemistry of all chiral atoms is as depicted. Compounds marked with (or) or (rel) are single enantiomers wherein the absolute stereochemistry was arbitrarily assigned (e.g., based on chiral SFC elution as described in the Examples section). Compounds marked with (and) or (rac) are mixtures of enantiomers wherein the relative stereochemistry is as shown. Compounds that have a stereogenic center where the configuration is not indicated in the structure as depicted and that are not marked in the “stereochemistry” column are mixtures of enantiomers. Compounds marked with (abs) are single enantiomers wherein the absolute stereochemistry is as indicated. In some instances, different indicators selected from (abs) (or) and (and) apply to different portions of the molecule. A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

TABLE 1
Exemplary compounds and biological data

| | | | Hap 1 | Hap1 | Hap1 | Hap1 | Hap1 |
|---|---|---|---|---|---|---|---|
| | | | MTAP | MTAP | MTAP | MTAP | MTAP |
| | | Stereo | intact | deleted | deleted | intact/ | deleted |
| STRUCTURE | Nr | chemistry | IC50 | IC50 | IC90 | deleted ratio | viability IC50 |
| 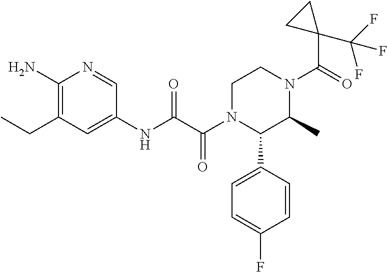 [SMILES: CCc1cc(NC(=O)C(=O)N2CCN(C(=O)C3(C(F)(F)F)CC3)[C@@H](C)[C@@H]2c2ccc(F)cc2)cnc1N] | 1 | (or) | c | bb | bbb | A | A* |
| 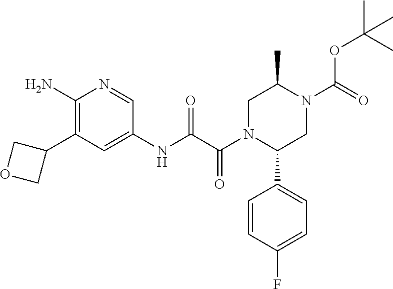 [SMILES: C[C@@H]1CN(C(=O)C(=O)Nc2cnc(N)c(C3COC3)c2)[C@@H](c2ccc(F)cc2)CN1C(=O)OC(C)(C)C] | 2 | (abs) | c | aa | aaa | C | A* |
| 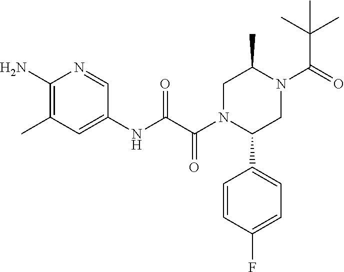 [SMILES: Cc1cc(NC(=O)C(=O)N2C[C@@H](C)N(C(=O)C(C)(C)C)C[C@@H]2c2ccc(F)cc2)cnc1N] | 3 | (abs) | b | aa | aaa | C | A* |
| 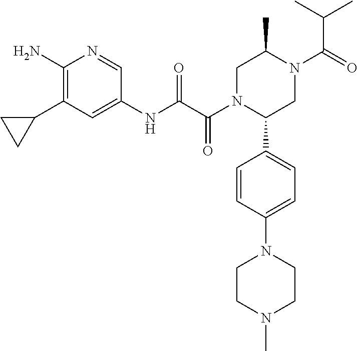 [SMILES: CC(C)C(=O)N1C[C@H](c2ccc(N3CCN(C)CC3)cc2)N(C(=O)C(=O)Nc2cnc(N)c(C3CC3)c2)C[C@H]1C] | 4 | (abs) | c | bb | ccc | C | B* |
| 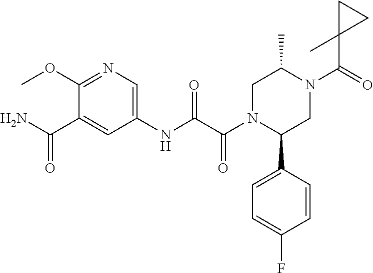 [SMILES: COc1ncc(NC(=O)C(=O)N2C[C@H](C)N(C(=O)C3(C)CC3)C[C@H]2c2ccc(F)cc2)cc1C(N)=O] | 5 | (or) | c | bb | aaa | A | B* |
| 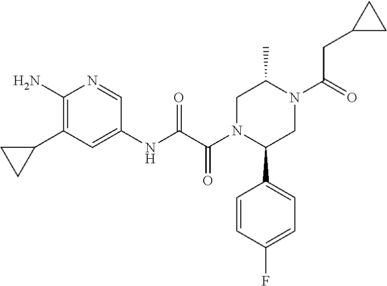 [SMILES: C[C@H]1CN(C(=O)C(=O)Nc2cnc(N)c(C3CC3)c2)[C@H](c2ccc(F)cc2)CN1C(=O)CC1CC1] | 6 | (or) | c | bb | bbb | B | A* |
| 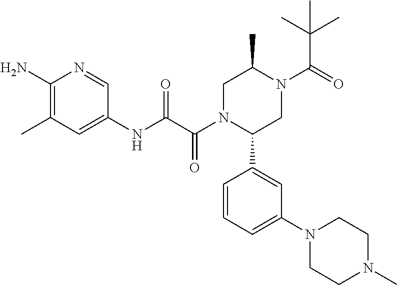 | 7 | (abs) | a | aa | aaa | C | A* |
| 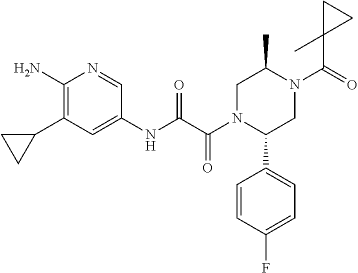 [SMILES: C[C@@H]1CN(C(=O)C(=O)Nc2cnc(N)c(C3CC3)c2)[C@@H](c2ccc(F)cc2)CN1C(=O)C1(C)CC1] | 8 | (abs) | b | aa | aaa | C | A* |
| 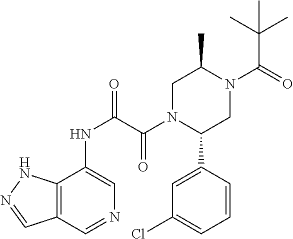 [SMILES: C[C@@H]1CN(C(=O)C(=O)Nc2cncc3cn[nH]c23)[C@@H](c2cccc(Cl)c2)CN1C(=O)C(C)(C)C] | 9 | (abs) | c | bb | ccc | B | B* |
| 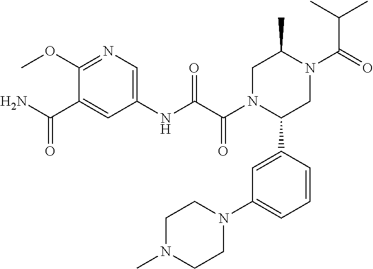 [SMILES: COc1ncc(NC(=O)C(=O)N2C[C@@H](C)N(C(=O)C(C)C)C[C@@H]2c2cccc(N3CCN(C)CC3)c2)cc1C(N)=O] | 10 | (abs) | c | bb | ccc | C | B* |
| 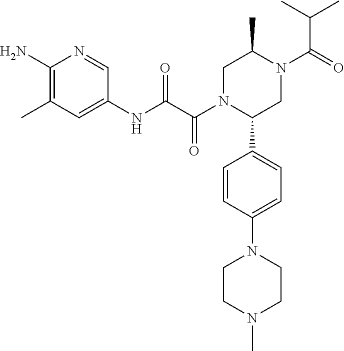 [SMILES: Cc1cc(NC(=O)C(=O)N2C[C@@H](C)N(C(=O)C(C)C)C[C@@H]2c2ccc(N3CCN(C)CC3)cc2)cnc1N] | 11 | (abs) | b | aa | aaa | C | A* |
| 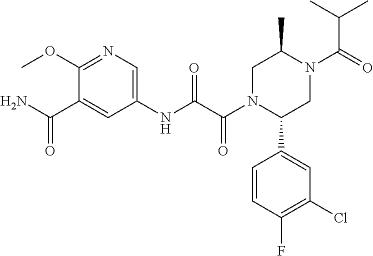 [SMILES: COc1ncc(NC(=O)C(=O)N2C[C@@H](C)N(C(=O)C(C)C)C[C@@H]2c2ccc(F)c(Cl)c2)cc1C(N)=O] | 12 | (abs) | c | bb | ccc | A | B* |
| 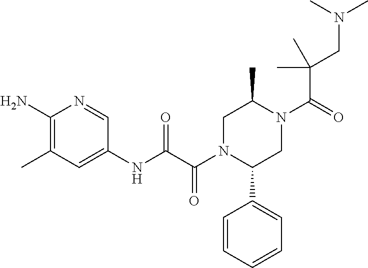 [SMILES: Cc1cc(NC(=O)C(=O)N2C[C@@H](C)N(C(=O)C(C)(C)CN(C)C)C[C@@H]2c2ccccc2)cnc1N] | 13 | (abs) | c | bb | aaa | C | A* |
| 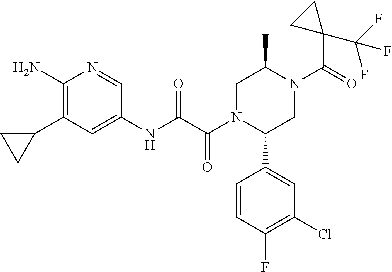 | 14 | (abs) | c | aa | aaa | C | A* |
| 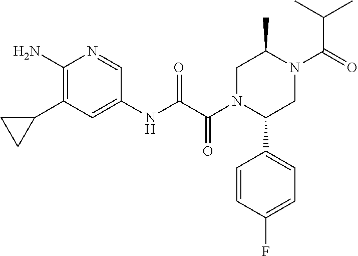 | 15 | (or) | | | | | C* |
| 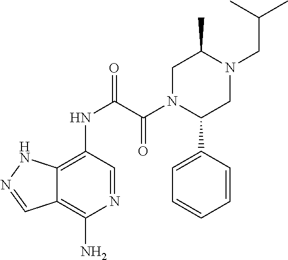 [SMILES: CC(C)CN1C[C@H](c2ccccc2)N(C(=O)C(=O)Nc2cnc(N)c3cn[nH]c23)C[C@H]1C] | 16 | (abs) | b | aa | aaa | A | A* |
| 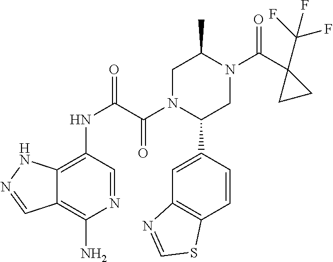 [SMILES: C[C@@H]1CN(C(=O)C(=O)Nc2cnc(N)c3cn[nH]c23)[C@@H](c2ccc3scnc3c2)CN1C(=O)C1(C(F)(F)F)CC1] | 17 | (abs) | | | | | |
| 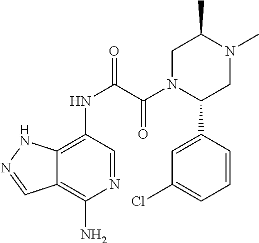 [SMILES: C[C@@H]1CN(C(=O)C(=O)Nc2cnc(N)c3cn[nH]c23)[C@@H](c2cccc(Cl)c2)CN1C] | 18 | (abs) | c | aa | aaa | A | A* |
| 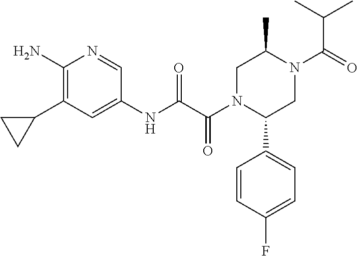 [SMILES: CC(C)C(=O)N1C[C@H](c2ccc(F)cc2)N(C(=O)C(=O)Nc2cnc(N)c(C3CC3)c2)C[C@H]1C] | 19 | (abs) | c | bb | aaa | C | A* |
| 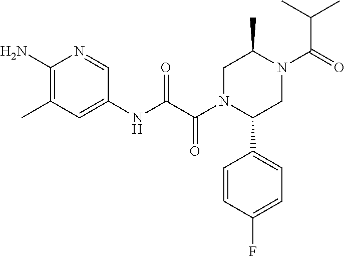 [SMILES: Cc1cc(NC(=O)C(=O)N2C[C@@H](C)N(C(=O)C(C)C)C[C@@H]2c2ccc(F)cc2)cnc1N] | 20 | (or) | c | aa | aaa | B | B* |
| 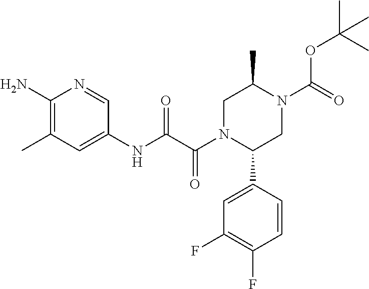 [SMILES: Cc1cc(NC(=O)C(=O)N2C[C@@H](C)N(C(=O)OC(C)(C)C)C[C@@H]2c2ccc(F)c(F)c2)cnc1N] | 21 | (abs) | c | bb | bbb | C | B* |
| 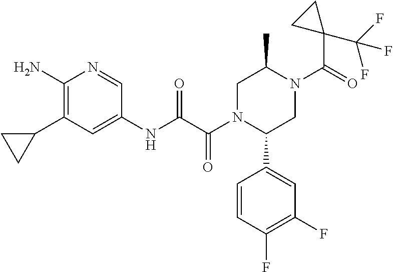 | 22 | (abs) | c | bb | bbb | B | A* |
| 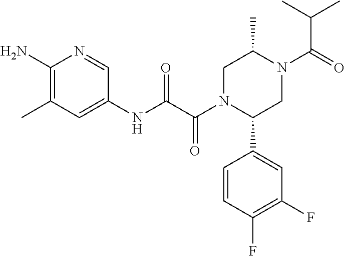 [SMILES: Cc1cc(NC(=O)C(=O)N2C[C@H](C)N(C(=O)C(C)C)C[C@@H]2c2ccc(F)c(F)c2)cnc1N] | 23 | (abs) | | | | | C* |
| 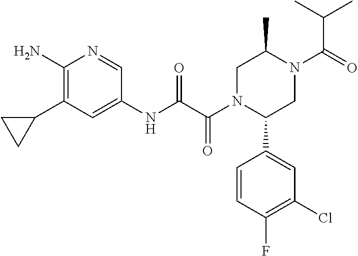 [SMILES: CC(C)C(=O)N1C[C@H](c2ccc(F)c(Cl)c2)N(C(=O)C(=O)Nc2cnc(N)c(C3CC3)c2)C[C@H]1C] | 24 | (abs) | c | aa | aaa | B | A* |
| 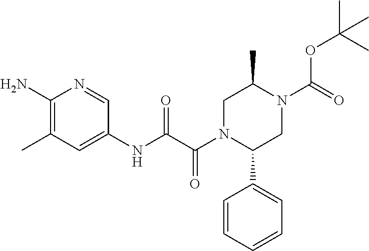 [SMILES: Cc1cc(NC(=O)C(=O)N2C[C@@H](C)N(C(=O)OC(C)(C)C)C[C@@H]2c2ccccc2)cnc1N] | 25 | (abs) | b | aa | aaa | C | A* |
| 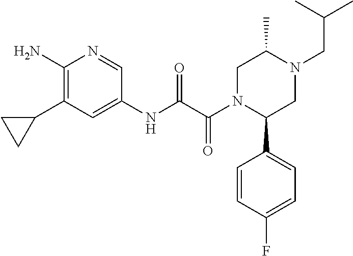 [SMILES: CC(C)CN1C[C@@H](c2ccc(F)cc2)N(C(=O)C(=O)Nc2cnc(N)c(C3CC3)c2)C[C@@H]1C] | 26 | (or) | | | | | C* |
| 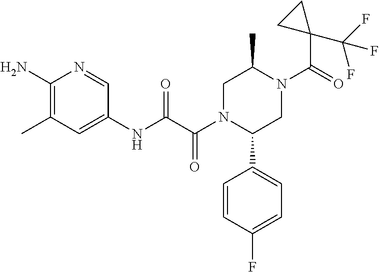 [SMILES: Cc1cc(NC(=O)C(=O)N2C[C@@H](C)N(C(=O)C3(C(F)(F)F)CC3)C[C@@H]2c2ccc(F)cc2)cnc1N] | 27 | (or) | c | bb | aaa | C | A* |
| 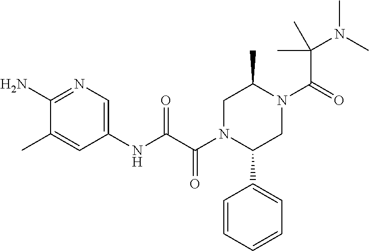 [SMILES: Cc1cc(NC(=O)C(=O)N2C[C@@H](C)N(C(=O)C(C)(C)N(C)C)C[C@@H]2c2ccccc2)cnc1N] | 28 | (abs) | c | bb | bbb | B | A* |
| 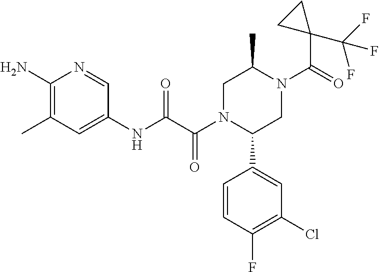 | 29 | (abs) | c | aa | aaa | B | A* |
| 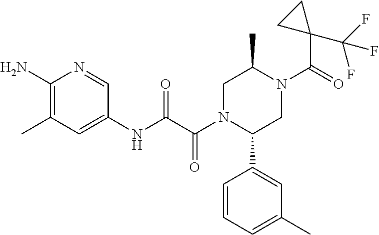 | 30 | (abs) | c | bb | bbb | C | A* |
| 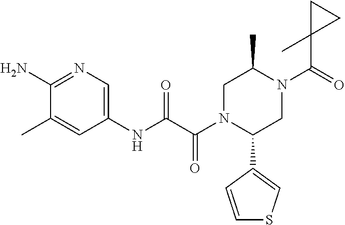 [SMILES: Cc1cc(NC(=O)C(=O)N2C[C@@H](C)N(C(=O)C3(C)CC3)C[C@@H]2c2ccsc2)cnc1N] | 31 | (abs) | c | aa | ccc | B | A* |
| 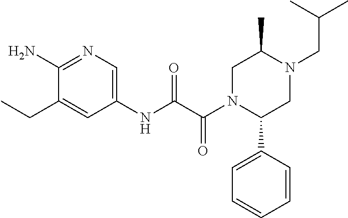 [SMILES: CCc1cc(NC(=O)C(=O)N2C[C@@H](C)N(CC(C)C)C[C@@H]2c2ccccc2)cnc1N] | 32 | (abs) | c | bb | ccc | A | A* |
| 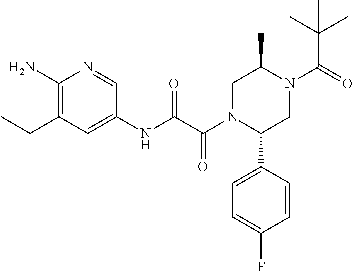 [SMILES: CCc1cc(NC(=O)C(=O)N2C[C@@H](C)N(C(=O)C(C)(C)C)C[C@@H]2c2ccc(F)cc2)cnc1N] | 33 | (abs) | b | aa | aaa | C | A* |
| 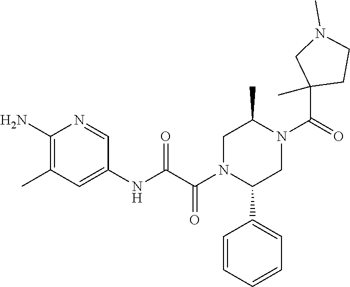 [SMILES: Cc1cc(NC(=O)C(=O)N2C[C@@H](C)N(C(=O)C3(C)CCN(C)C3)C[C@@H]2c2ccccc2)cnc1N] | 34 | (abs) | c | bb | ccc | B | B* |
| 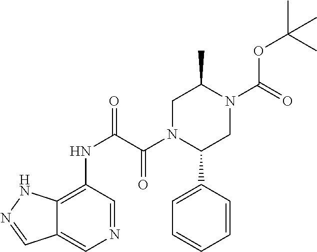 [SMILES: C[C@@H]1CN(C(=O)C(=O)Nc2cncc3cn[nH]c23)[C@@H](c2ccccc2)CN1C(=O)OC(C)(C)C] | 35 | (abs) | c | bb | ccc | A | A* |
| 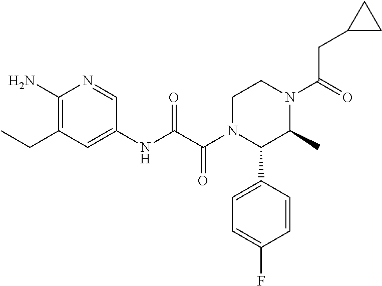 [SMILES: CCc1cc(NC(=O)C(=O)N2CCN(C(=O)CC3CC3)[C@@H](C)[C@@H]2c2ccc(F)cc2)cnc1N] | 36 | (or) | | | | | C* |
| 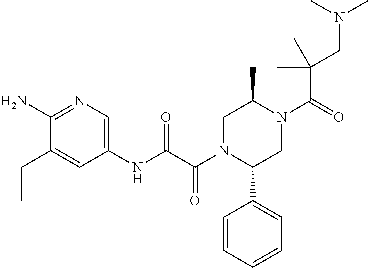 [SMILES: CCc1cc(NC(=O)C(=O)N2C[C@@H](C)N(C(=O)C(C)(C)CN(C)C)C[C@@H]2c2ccccc2)cnc1N] | 37 | (abs) | c | aa | aaa | A | A* |
| 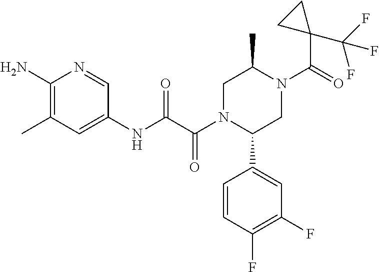 | 38 | (abs) | c | bb | bbb | C | A* |
| 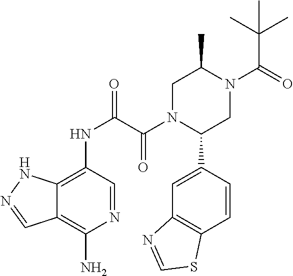 [SMILES: C[C@@H]1CN(C(=O)C(=O)Nc2cnc(N)c3cn[nH]c23)[C@@H](c2ccc3scnc3c2)CN1C(=O)C(C)(C)C] | 39 | (abs) | | | | | |
| 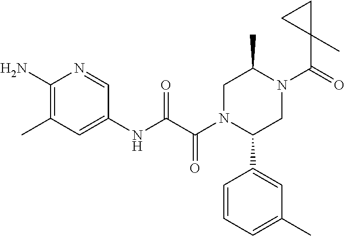 | 40 | (abs) | c | bb | ccc | C | A* |
| 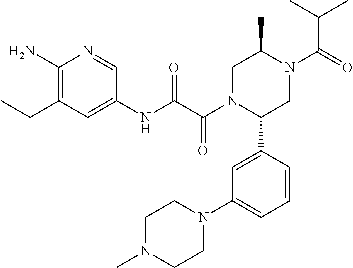 [SMILES: CCc1cc(NC(=O)C(=O)N2C[C@@H](C)N(C(=O)C(C)C)C[C@@H]2c2cccc(N3CCN(C)CC3)c2)cnc1N] | 41 | (abs) | b | aa | aaa | C | A* |
| 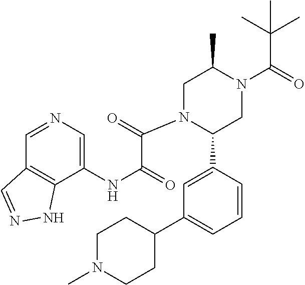 [SMILES: C[C@@H]1CN(C(=O)C(=O)Nc2cncc3cn[nH]c23)[C@@H](c2cccc(C3CCN(C)CC3)c2)CN1C(=O)C(C)(C)C] | 42 | (abs) | b | bb | bbb | C | A* |
| 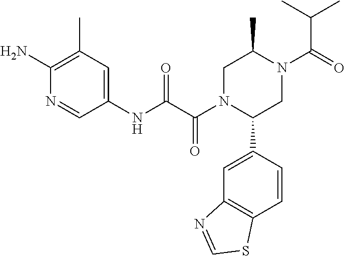 [SMILES: Cc1cc(NC(=O)C(=O)N2C[C@@H](C)N(C(=O)C(C)C)C[C@@H]2c2ccc3scnc3c2)cnc1N] | 43 | (or) | c | bb | ccc | C | B* |
| 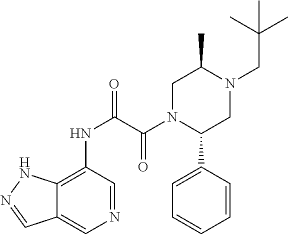 [SMILES: C[C@@H]1CN(C(=O)C(=O)Nc2cncc3cn[nH]c23)[C@@H](c2ccccc2)CN1CC(C)(C)C] | 44 | (abs) | c | aa | aaa | A | B* |
| 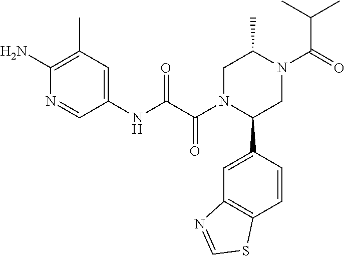 [SMILES: Cc1cc(NC(=O)C(=O)N2C[C@H](C)N(C(=O)C(C)C)C[C@H]2c2ccc3scnc3c2)cnc1N] | 45 | (or) | C | cc | ccc | C | C* |
| 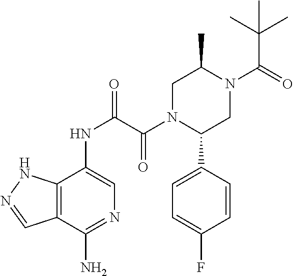 [SMILES: C[C@@H]1CN(C(=O)C(=O)Nc2cnc(N)c3cn[nH]c23)[C@@H](c2ccc(F)cc2)CN1C(=O)C(C)(C)C] | 46 | (abs) | b | aa | aaa | C | A* |
| 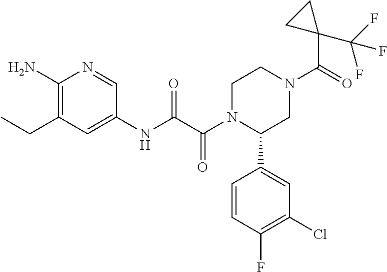 | 47 | (or) | c | cc | ccc | C | C* |
| 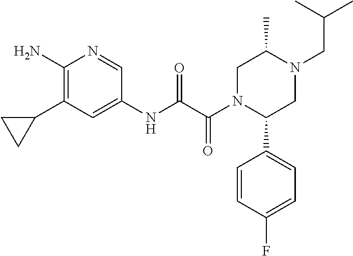 [SMILES: CC(C)CN1C[C@H](c2ccc(F)cc2)N(C(=O)C(=O)Nc2cnc(N)c(C3CC3)c2)C[C@@H]1C] | 48 | (or) | | | | | C* |
| 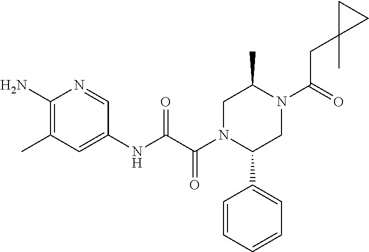 [SMILES: Cc1cc(NC(=O)C(=O)N2C[C@@H](C)N(C(=O)CC3(C)CC3)C[C@@H]2c2ccccc2)cnc1N] | 49 | (abs) | c | bb | bbb | C | B* |
| 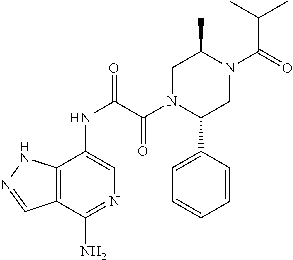 [SMILES: CC(C)C(=O)N1C[C@H](c2ccccc2)N(C(=O)C(=O)Nc2cnc(N)c3cn[nH]c23)C[C@H]1C] | 50 | (abs) | b | aa | aaa | C | A* |
| 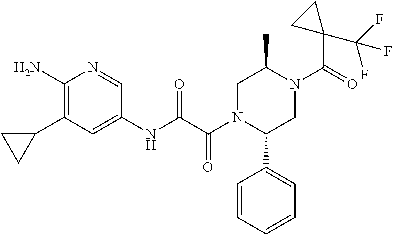 | 51 | (abs) | c | bb | aaa | C | A* |
| 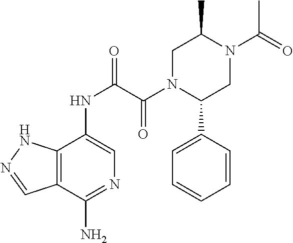 [SMILES: CC(=O)N1C[C@H](c2ccccc2)N(C(=O)C(=O)Nc2cnc(N)c3cn[nH]c23)C[C@H]1C] | 52 | (abs) | c | bb | aaa | B | A* |
| 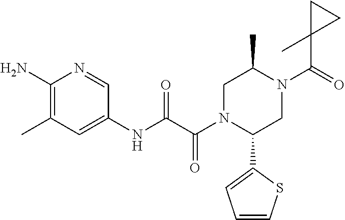 [SMILES: Cc1cc(NC(=O)C(=O)N2C[C@@H](C)N(C(=O)C3(C)CC3)C[C@@H]2c2cccs2)cnc1N] | 53 | (abs) | b | aa | aaa | C | A* |
| 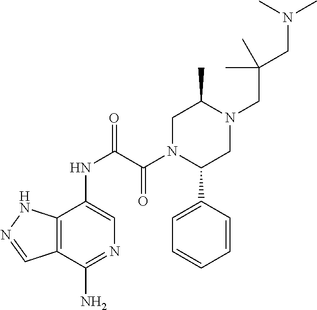 [SMILES: C[C@@H]1CN(C(=O)C(=O)Nc2cnc(N)c3cn[nH]c23)[C@@H](c2ccccc2)CN1CC(C)(C)CN(C)C] | 54 | (abs) | b | bb | aaa | C | A* |
| 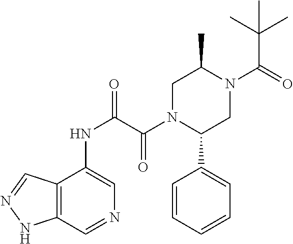 [SMILES: C[C@@H]1CN(C(=O)C(=O)Nc2cncc3[nH]ncc23)[C@@H](c2ccccc2)CN1C(=O)C(C)(C)C] | 55 | (abs) | c | bb | bbb | C | A* |
| 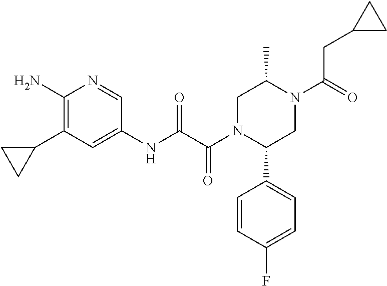 [SMILES: C[C@H]1CN(C(=O)C(=O)Nc2cnc(N)c(C3CC3)c2)[C@@H](c2ccc(F)cc2)CN1C(=O)CC1CC1] | 56 | (or) | | | | | C* |
| 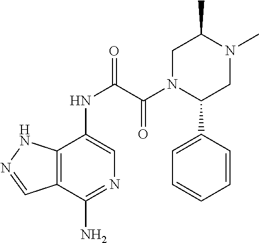 [SMILES: C[C@@H]1CN(C(=O)C(=O)Nc2cnc(N)c3cn[nH]c23)[C@@H](c2ccccc2)CN1C] | 57 | (abs) | c | aa | aaa | B | A* |
| 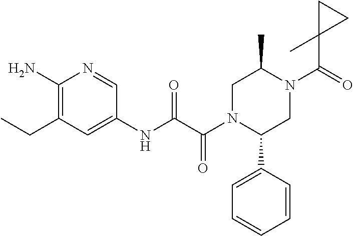 | 58 | (abs) | c | bb | aaa | C | A* |
| 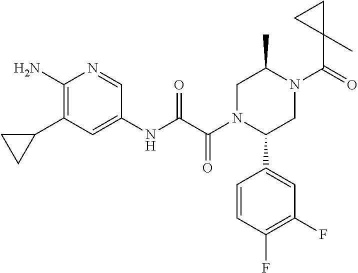 [SMILES: C[C@@H]1CN(C(=O)C(=O)Nc2cnc(N)c(C3CC3)c2)[C@@H](c2ccc(F)c(F)c2)CN1C(=O)C1(C)CC1] | 59 | (abs) | c | aa | aaa | B | A* |
| 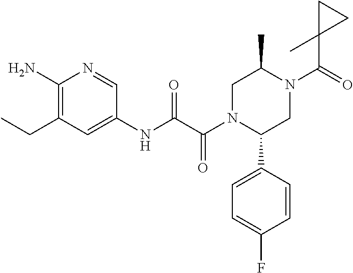 [SMILES: CCc1cc(NC(=O)C(=O)N2C[C@@H](C)N(C(=O)C3(C)CC3)C[C@@H]2c2ccc(F)cc2)cnc1N] | 60 | (abs) | b | aa | aaa | C | A* |
| 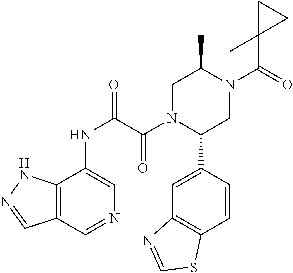 [SMILES: C[C@@H]1CN(C(=O)C(=O)Nc2cncc3cn[nH]c23)[C@@H](c2ccc3scnc3c2)CN1C(=O)C1(C)CC1] | 61 | (abs) | c | bb | bbb | C | B* |
| 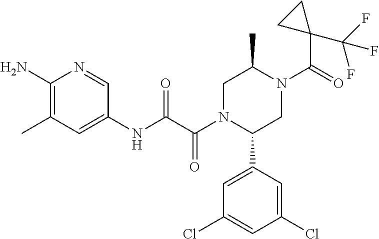 | 62 | (abs) | c | bb | bbb | A | A* |
| 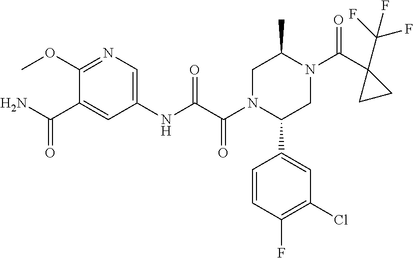 [SMILES: COc1ncc(NC(=O)C(=O)N2C[C@@H](C)N(C(=O)C3(C(F)(F)F)CC3)C[C@@H]2c2ccc(F)c(Cl)c2)cc1C(N)=O] | 63 | (abs) | c | bb | bbb | A | B* |
| 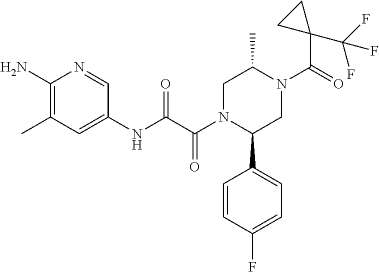 [SMILES: Cc1cc(NC(=O)C(=O)N2C[C@H](C)N(C(=O)C3(C(F)(F)F)CC3)C[C@H]2c2ccc(F)cc2)cnc1N] | 64 | (or) | c | cc | ccc | C | C* |
| 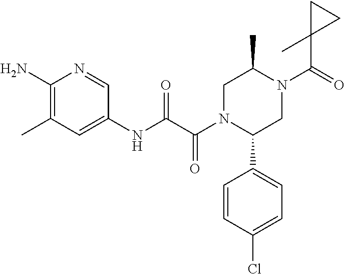 [SMILES: Cc1cc(NC(=O)C(=O)N2C[C@@H](C)N(C(=O)C3(C)CC3)C[C@@H]2c2ccc(Cl)cc2)cnc1N] | 65 | (abs) | b | aa | aaa | C | A* |
| 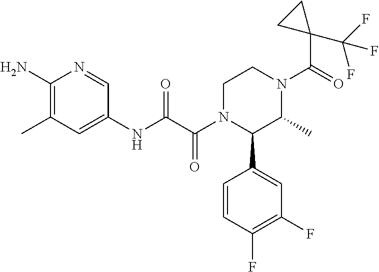 [SMILES: Cc1cc(NC(=O)C(=O)N2CCN(C(=O)C3(C(F)(F)F)CC3)[C@H](C)[C@H]2c2ccc(F)c(F)c2)cnc1N] | 66 | (or) | c | bb | ccc | A | B* |
| 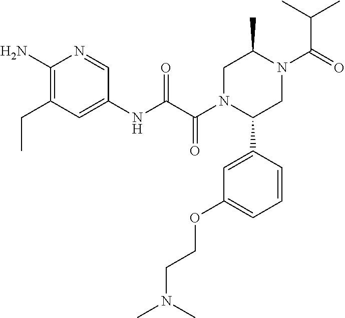 [SMILES: CCc1cc(NC(=O)C(=O)N2C[C@@H](C)N(C(=O)C(C)C)C[C@@H]2c2cccc(OCCN(C)C)c2)cnc1N] | 67 | (abs) | b | aa | aaa | C | A* |
| 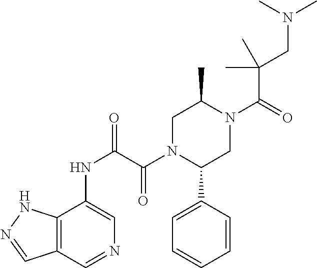 [SMILES: C[C@@H]1CN(C(=O)C(=O)Nc2cncc3cn[nH]c23)[C@@H](c2ccccc2)CN1C(=O)C(C)(C)CN(C)C] | 68 | (abs) | c | bb | bbb | A | B* |
| 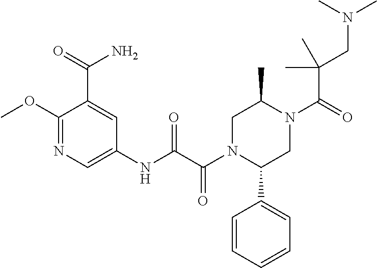 | 69 | (abs) | c | bb | ccc | A | B* |
| 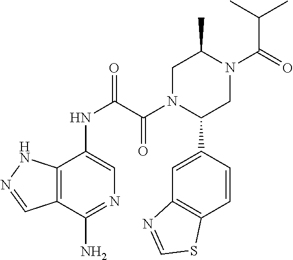 [SMILES: CC(C)C(=O)N1C[C@H](c2ccc3scnc3c2)N(C(=O)C(=O)Nc2cnc(N)c3cn[nH]c23)C[C@H]1C] | 70 | (abs) | | | | | |
| 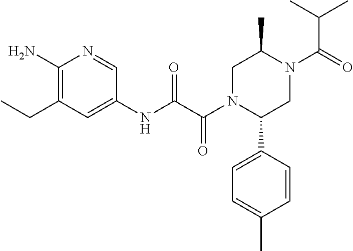 [SMILES: CCc1cc(NC(=O)C(=O)N2C[C@@H](C)N(C(=O)C(C)C)C[C@@H]2c2ccc(C)cc2)cnc1N] | 71 | (abs) | c | bb | bbb | B | A* |
| 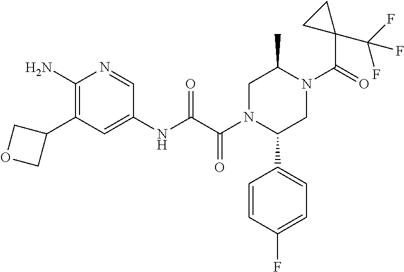 | 72 | (abs) | c | bb | bbb | A | A* |
| 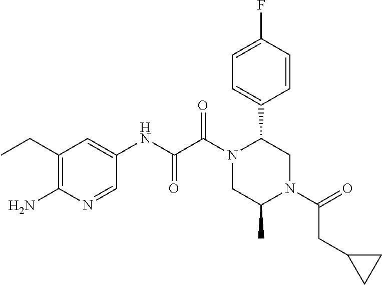 [SMILES: CCc1cc(NC(=O)C(=O)N2C[C@H](C)N(C(=O)CC3CC3)C[C@H]2c2ccc(F)cc2)cnc1N] | 73 | (or) | c | bb | bbb | B | A* |
| 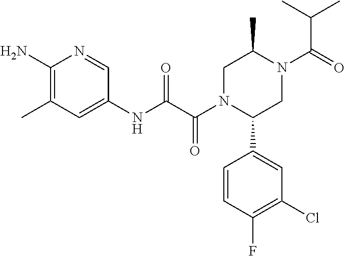 [SMILES: Cc1cc(NC(=O)C(=O)N2C[C@@H](C)N(C(=O)C(C)C)C[C@@H]2c2ccc(F)c(Cl)c2)cnc1N] | 74 | (abs) | c | bb | bbb | A | B* |
| 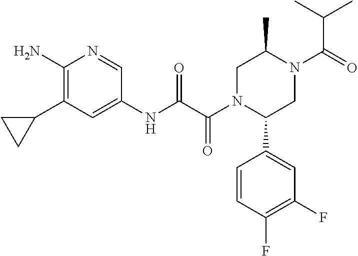 [SMILES: CC(C)C(=O)N1C[C@H](c2ccc(F)c(F)c2)N(C(=O)C(=O)Nc2cnc(N)c(C3CC3)c2)C[C@H]1C] | 75 | (abs) | c | bb | ccc | C | A* |
| 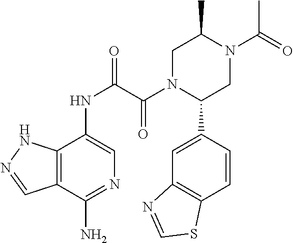 [SMILES: CC(=O)N1C[C@H](c2ccc3scnc3c2)N(C(=O)C(=O)Nc2cnc(N)c3cn[nH]c23)C[C@H]1C] | 76 | (abs) | | | | | |
| 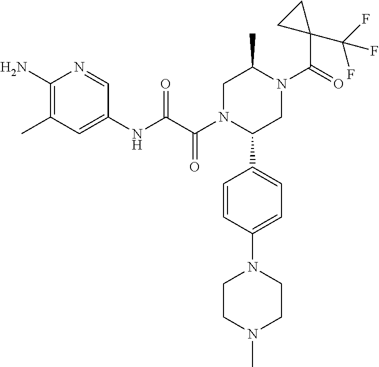 [SMILES: Cc1cc(NC(=O)C(=O)N2C[C@@H](C)N(C(=O)C3(C(F)(F)F)CC3)C[C@@H]2c2ccc(N3CCN(C)CC3)cc2)cnc1N] | 77 | (abs) | b | aa | aaa | B | A* |
| 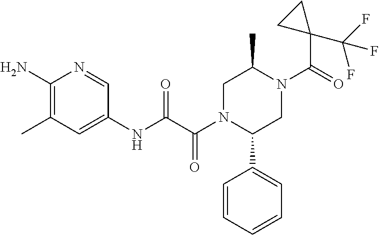 | 78 | (abs) | c | bb | ccc | B | A* |
| 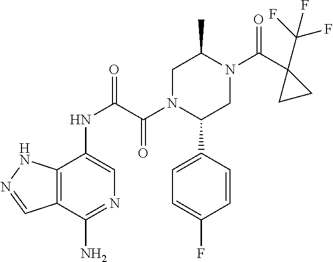 [SMILES: C[C@@H]1CN(C(=O)C(=O)Nc2cnc(N)c3cn[nH]c23)[C@@H](c2ccc(F)cc2)CN1C(=O)C1(C(F)(F)F)CC1] | 79 | (abs) | b | aa | aaa | C | A* |
| 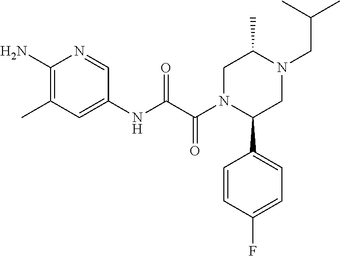 [SMILES: Cc1cc(NC(=O)C(=O)N2C[C@H](C)N(CC(C)C)C[C@H]2c2ccc(F)cc2)cnc1N] | 80 | (abs) | c | bb | ccc | C | B* |
| 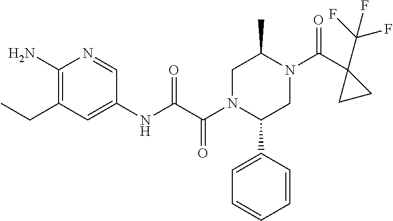 | 81 | (abs) | c | bb | bbb | B | A* |
| 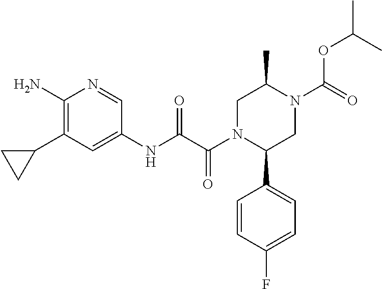 [SMILES: CC(C)OC(=O)N1C[C@@H](c2ccc(F)cc2)N(C(=O)C(=O)Nc2cnc(N)c(C3CC3)c2)C[C@H]1C] | 82 | (or) | | | | | C* |
| 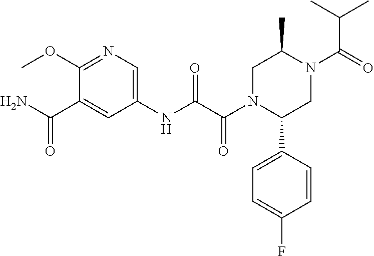 [SMILES: COc1ncc(NC(=O)C(=O)N2C[C@@H](C)N(C(=O)C(C)C)C[C@@H]2c2ccc(F)cc2)cc1C(N)=O] | 83 | (or) | c | bb | bbb | A | B* |
| 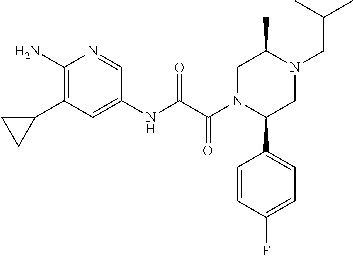 [SMILES: CC(C)CN1C[C@@H](c2ccc(F)cc2)N(C(=O)C(=O)Nc2cnc(N)c(C3CC3)c2)C[C@H]1C] | 84 | (or) | c | bb | aaa | A | A* |
| 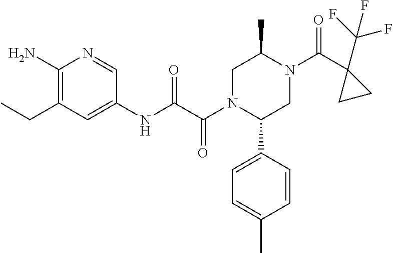 [SMILES: CCc1cc(NC(=O)C(=O)N2C[C@@H](C)N(C(=O)C3(C(F)(F)F)CC3)C[C@@H]2c2ccc(C)cc2)cnc1N] | 85 | (abs) | c | bb | bbb | B | A* |
| 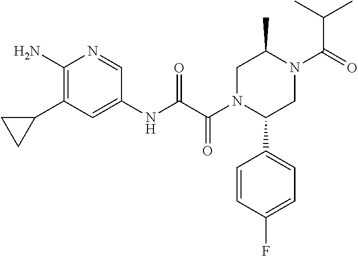 [SMILES: CC(C)C(=O)N1C[C@H](c2ccc(F)cc2)N(C(=O)C(=O)Nc2cnc(N)c(C3CC3)c2)C[C@H]1C] | 86 | (or) | c | bb | aaa | C | A* |
| 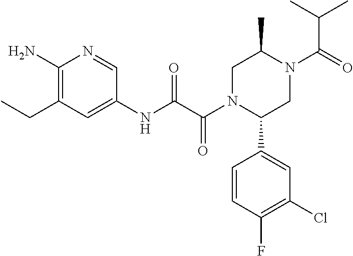 [SMILES: CCc1cc(NC(=O)C(=O)N2C[C@@H](C)N(C(=O)C(C)C)C[C@@H]2c2ccc(F)c(Cl)c2)cnc1N] | 87 | (abs) | c | aa | ccc | A | A* |
| 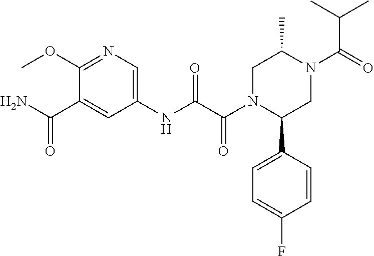 [SMILES: COc1ncc(NC(=O)C(=O)N2C[C@H](C)N(C(=O)C(C)C)C[C@H]2c2ccc(F)cc2)cc1C(N)=O] | 88 | (or) | c | cc | ccc | C | C* |
| 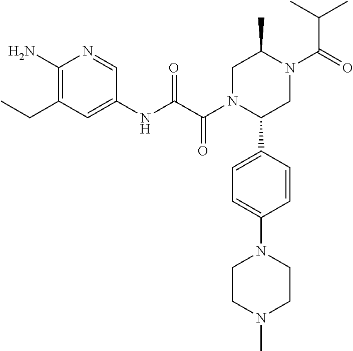 [SMILES: CCc1cc(NC(=O)C(=O)N2C[C@@H](C)N(C(=O)C(C)C)C[C@@H]2c2ccc(N3CCN(C)CC3)cc2)cnc1N] | 89 | (abs) | c | aa | aaa | A | A* |
| 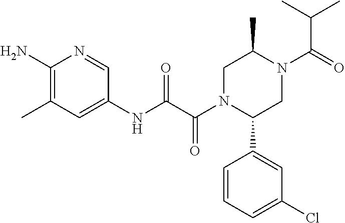 | 90 | (abs) | c | bb | ccc | B | A* |
| 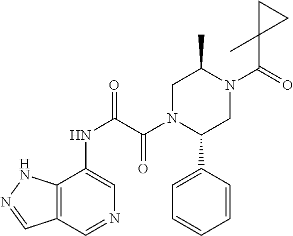 [SMILES: C[C@@H]1CN(C(=O)C(=O)Nc2cncc3cn[nH]c23)[C@@H](c2ccccc2)CN1C(=O)C1(C)CC1] | 91 | (abs) | c | bb | ccc | B | A* |
| 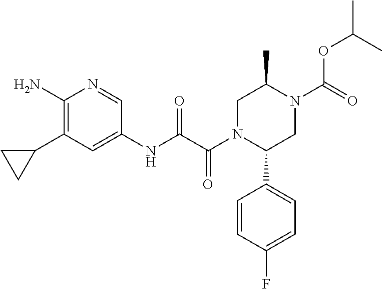 [SMILES: CC(C)OC(=O)N1C[C@H](c2ccc(F)cc2)N(C(=O)C(=O)Nc2cnc(N)c(C3CC3)c2)C[C@H]1C] | 92 | (or) | c | cc | ccc | C | C* |
| 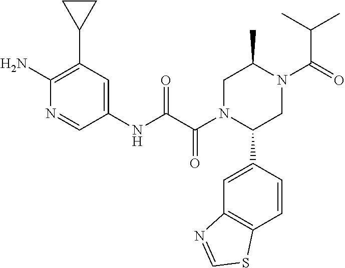 [SMILES: CC(C)C(=O)N1C[C@H](c2ccc3scnc3c2)N(C(=O)C(=O)Nc2cnc(N)c(C3CC3)c2)C[C@H]1C] | 93 | (abs) | b | bb | aaa | C | A* |
| 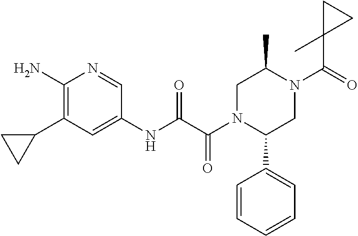 | 94 | (abs) | c | aa | bbb | B | A* |
| 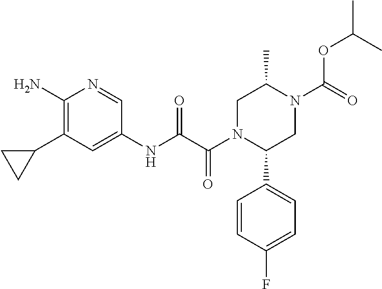 [SMILES: CC(C)OC(=O)N1C[C@H](c2ccc(F)cc2)N(C(=O)C(=O)Nc2cnc(N)c(C3CC3)c2)C[C@@H]1C] | 95 | (or) | | | | | C* |
| 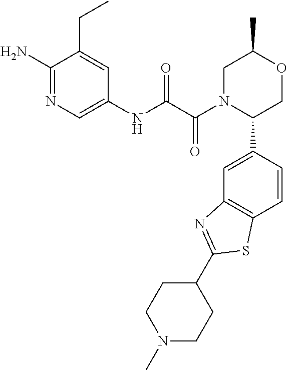 [SMILES: CCc1cc(NC(=O)C(=O)N2C[C@@H](C)OC[C@@H]2c2ccc3sc(C4CCN(C)CC4)nc3c2)cnc1N] | 96 | (abs) | c | bb | bbb | B | A* |
| 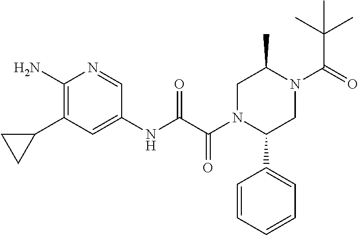 [SMILES: C[C@@H]1CN(C(=O)C(=O)Nc2cnc(N)c(C3CC3)c2)[C@@H](c2ccccc2)CN1C(=O)C(C)(C)C] | 97 | (abs) | b | aa | aaa | C | A* |
| 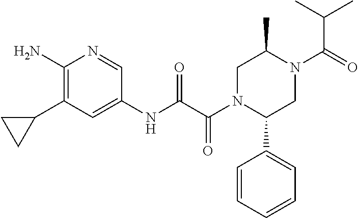 [SMILES: CC(C)C(=O)N1C[C@H](c2ccccc2)N(C(=O)C(=O)Nc2cnc(N)c(C3CC3)c2)C[C@H]1C] | 98 | (abs) | c | bb | bbb | C | A* |
| 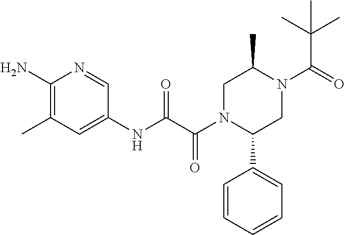 [SMILES: Cc1cc(NC(=O)C(=O)N2C[C@@H](C)N(C(=O)C(C)(C)C)C[C@@H]2c2ccccc2)cnc1N] | 99 | (abs) | b | aa | aaa | C | A* |
| 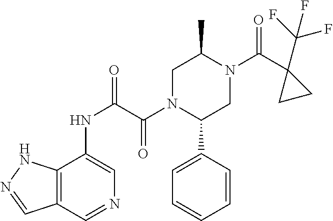 [SMILES: C[C@@H]1CN(C(=O)C(=O)Nc2cncc3cn[nH]c23)[C@@H](c2ccccc2)CN1C(=O)C1(C(F)(F)F)CC1] | 100 | (abs) | c | bb | ccc | C | A* |
| 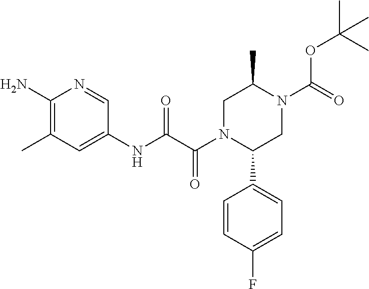 [SMILES: Cc1cc(NC(=O)C(=O)N2C[C@@H](C)N(C(=O)OC(C)(C)C)C[C@@H]2c2ccc(F)cc2)cnc1N] | 101 | (abs) | c | aa | aaa | B | A* |
| 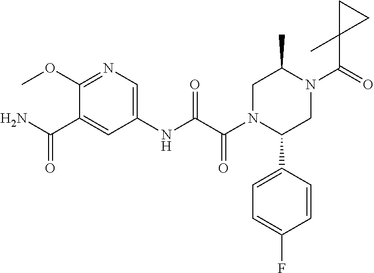 [SMILES: COc1ncc(NC(=O)C(=O)N2C[C@@H](C)N(C(=O)C3(C)CC3)C[C@@H]2c2ccc(F)cc2)cc1C(N)=O] | 102 | (or) | c | cc | ccc | C | C* |
| 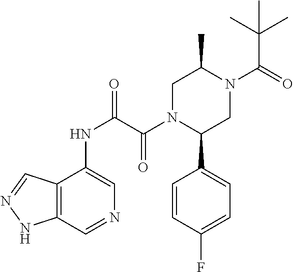 [SMILES: C[C@@H]1CN(C(=O)C(=O)Nc2cncc3[nH]ncc23)[C@H](c2ccc(F)cc2)CN1C(=O)C(C)(C)C] | 103 | (abs) | | | | | C* |
| 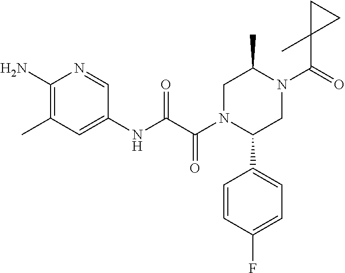 [SMILES: Cc1cc(NC(=O)C(=O)N2C[C@@H](C)N(C(=O)C3(C)CC3)C[C@@H]2c2ccc(F)cc2)cnc1N] | 104 | (abs) | b | aa | bbb | C | A* |
| 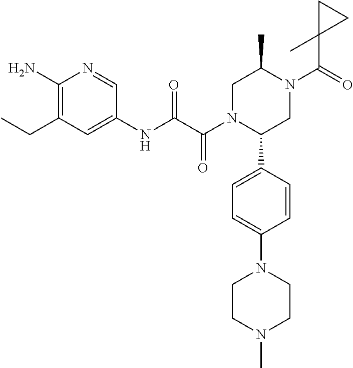 [SMILES: CCc1cc(NC(=O)C(=O)N2C[C@@H](C)N(C(=O)C3(C)CC3)C[C@@H]2c2ccc(N3CCN(C)CC3)cc2)cnc1N] | 105 | (abs) | c | aa | aaa | B | A* |
| 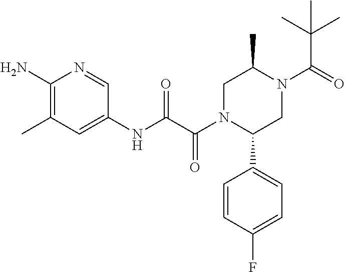 [SMILES: Cc1cc(NC(=O)C(=O)N2C[C@@H](C)N(C(=O)C(C)(C)C)C[C@@H]2c2ccc(F)cc2)cnc1N] | 106 | (abs) | c | bb | aaa | C | A* |
| 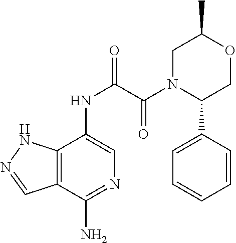 [SMILES: C[C@@H]1CN(C(=O)C(=O)Nc2cnc(N)c3cn[nH]c23)[C@@H](c2ccccc2)CO1] | 107 | (abs) | c | aa | aaa | A | B* |
| 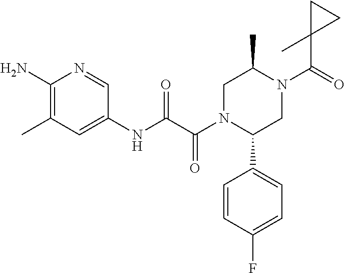 [SMILES: Cc1cc(NC(=O)C(=O)N2C[C@@H](C)N(C(=O)C3(C)CC3)C[C@@H]2c2ccc(F)cc2)cnc1N] | 108 | (abs) | c | bb | bbb | C | A* |
| 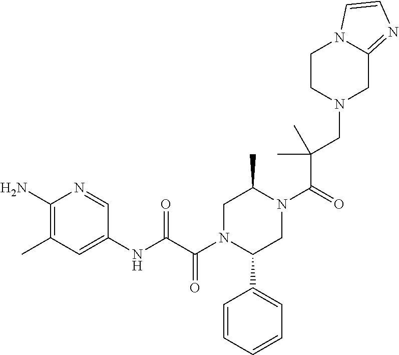 [SMILES: Cc1cc(NC(=O)C(=O)N2C[C@@H](C)N(C(=O)C(C)(C)CN3CCn4ccnc4C3)C[C@@H]2c2ccccc2)cnc1N] | 109 | (abs) | c | bb | ccc | C | B* |
| 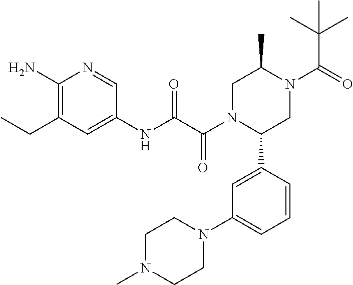 [SMILES: CCc1cc(NC(=O)C(=O)N2C[C@@H](C)N(C(=O)C(C)(C)C)C[C@@H]2c2cccc(N3CCN(C)CC3)c2)cnc1N] | 110 | (abs) | a | aa | aaa | C | A* |
| 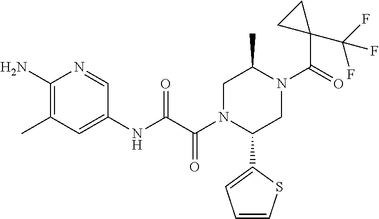 [SMILES: Cc1cc(NC(=O)C(=O)N2C[C@@H](C)N(C(=O)C3(C(F)(F)F)CC3)C[C@@H]2c2cccs2)cnc1N] | 111 | (abs) | b | aa | bbb | C | A* |
| 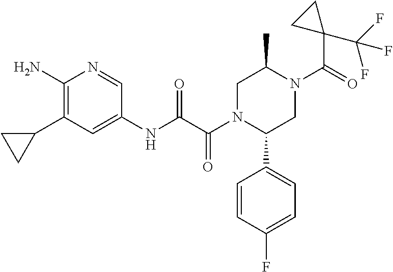 [SMILES: C[C@@H]1CN(C(=O)C(=O)Nc2cnc(N)c(C3CC3)c2)[C@@H](c2ccc(F)cc2)CN1C(=O)C1(C(F)(F)F)CC1] | 112 | (abs) | c | bb | bbb | C | A* |
| 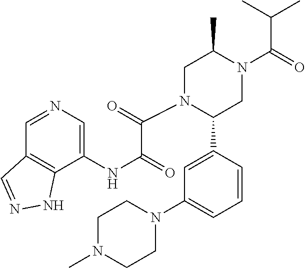 [SMILES: CC(C)C(=O)N1C[C@H](c2cccc(N3CCN(C)CC3)c2)N(C(=O)C(=O)Nc2cncc3cn[nH]c23)C[C@H]1C] | 113 | (abs) | c | bb | ccc | C | A* |
| 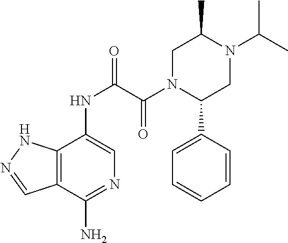 [SMILES: CC(C)N1C[C@H](c2ccccc2)N(C(=O)C(=O)Nc2cnc(N)c3cn[nH]c23)C[C@H]1C] | 114 | (abs) | | | | | |
| 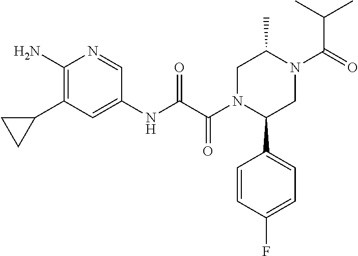 [SMILES: CC(C)C(=O)N1C[C@@H](c2ccc(F)cc2)N(C(=O)C(=O)Nc2cnc(N)c(C3CC3)c2)C[C@@H]1C] | 115 | (or) | c | cc | ccc | C | C* |
| 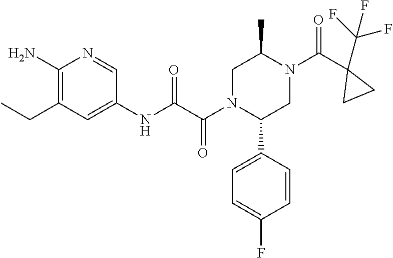 | 116 | (abs) | c | aa | aaa | A | A* |
| 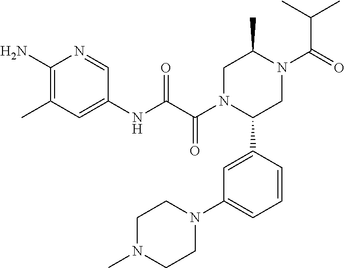 | 117 | (abs) | b | bb | aaa | C | A* |
| 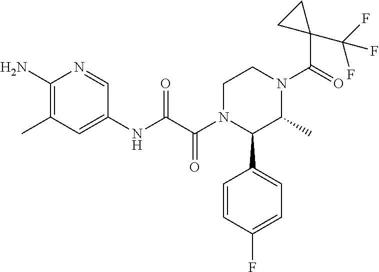 [SMILES: Cc1cc(NC(=O)C(=O)N2CCN(C(=O)C3(C(F)(F)F)CC3)[C@H](C)[C@H]2c2ccc(F)cc2)cnc1N] | 118 | (abs) | c | bb | ccc | B | B* |
| 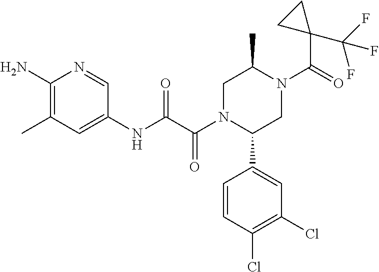 [SMILES: Cc1cc(NC(=O)C(=O)N2C[C@@H](C)N(C(=O)C3(C(F)(F)F)CC3)C[C@@H]2c2ccc(Cl)c(Cl)c2)cnc1N] | 119 | (abs) | c | bb | bbb | C | A* |
| 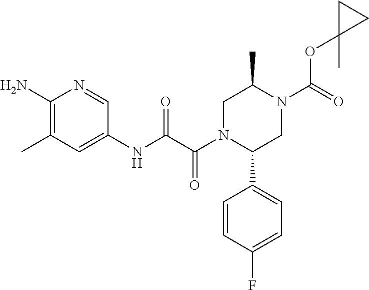 [SMILES: Cc1cc(NC(=O)C(=O)N2C[C@@H](C)N(C(=O)OC3(C)CC3)C[C@@H]2c2ccc(F)cc2)cnc1N] | 120 | (abs) | c | bb | bbb | B | B* |
| 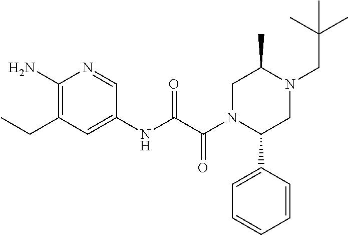 [SMILES: CCc1cc(NC(=O)C(=O)N2C[C@@H](C)N(CC(C)(C)C)C[C@@H]2c2ccccc2)cnc1N] | 121 | (abs) | c | bb | bbb | B | A* |
| 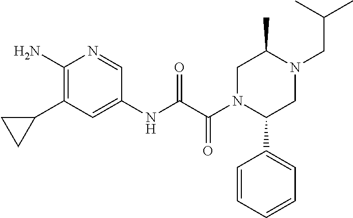 [SMILES: CC(C)CN1C[C@H](c2ccccc2)N(C(=O)C(=O)Nc2cnc(N)c(C3CC3)c2)C[C@H]1C] | 122 | (abs) | c | bb | ccc | B | A* |
| 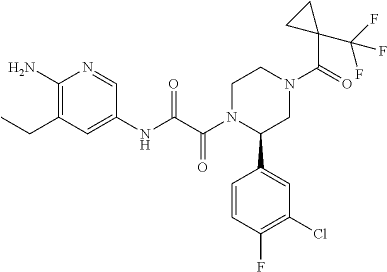 | 123 | (or) | c | bb | ccc | A | B* |
| 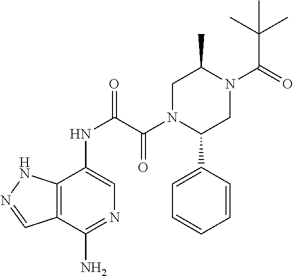 [SMILES: C[C@@H]1CN(C(=O)C(=O)Nc2cnc(N)c3cn[nH]c23)[C@@H](c2ccccc2)CN1C(=O)C(C)(C)C] | 124 | (abs) | a | aa | aaa | C | A* |
| 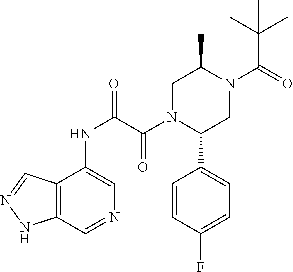 [SMILES: C[C@@H]1CN(C(=O)C(=O)Nc2cncc3[nH]ncc23)[C@@H](c2ccc(F)cc2)CN1C(=O)C(C)(C)C] | 125 | (abs) | c | bb | aaa | A | B* |
| 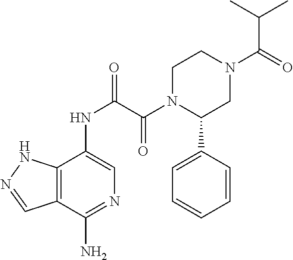 [SMILES: CC(C)C(=O)N1CCN(C(=O)C(=O)Nc2cnc(N)c3cn[nH]c23)[C@@H](c2ccccc2)C1] | 126 | (abs) | c | aa | aaa | A | B* |
| 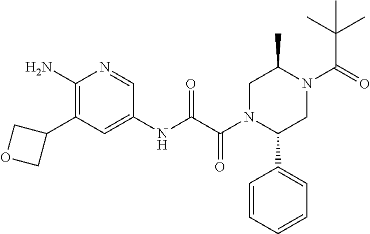 [SMILES: C[C@@H]1CN(C(=O)C(=O)Nc2cnc(N)c(C3COC3)c2)[C@@H](c2ccccc2)CN1C(=O)C(C)(C)C] | 127 | (abs) | b | bb | aaa | C | A* |
| 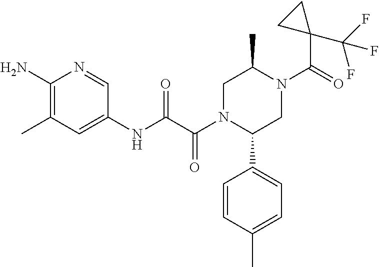 | 128 | (abs) | c | bb | bbb | B | A* |
| 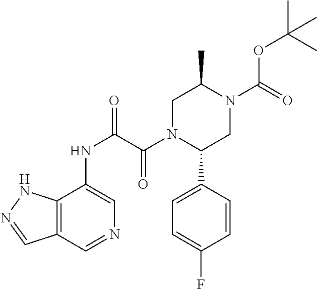 [SMILES: C[C@@H]1CN(C(=O)C(=O)Nc2cncc3cn[nH]c23)[C@@H](c2ccc(F)cc2)CN1C(=O)OC(C)(C)C] | 129 | (abs) | c | bb | ccc | A | B* |
| 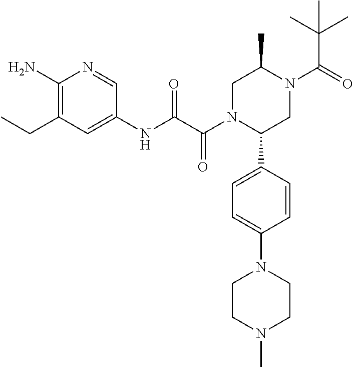 [SMILES: CCc1cc(NC(=O)C(=O)N2C[C@@H](C)N(C(=O)C(C)(C)C)C[C@@H]2c2ccc(N3CCN(C)CC3)cc2)cnc1N] | 130 | (abs) | b | aa | aaa | C | A* |
| 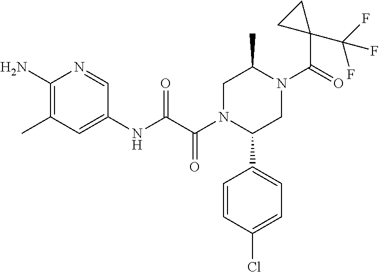 [SMILES: Cc1cc(NC(=O)C(=O)N2C[C@@H](C)N(C(=O)C3(C(F)(F)F)CC3)C[C@@H]2c2ccc(Cl)cc2)cnc1N] | 131 | (abs) | c | aa | bbb | C | B* |
| 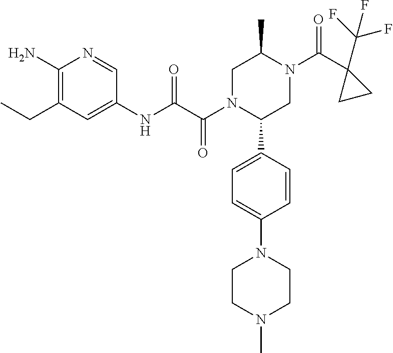 [SMILES: CCc1cc(NC(=O)C(=O)N2C[C@@H](C)N(C(=O)C3(C(F)(F)F)CC3)C[C@@H]2c2ccc(N3CCN(C)CC3)cc2)cnc1N] | 132 | (abs) | b | aa | aaa | C | A* |
| 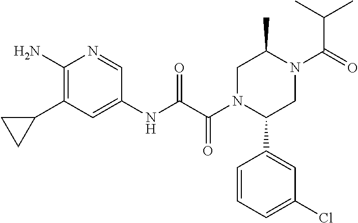 | 133 | (abs) | c | bb | aaa | B | A* |
| 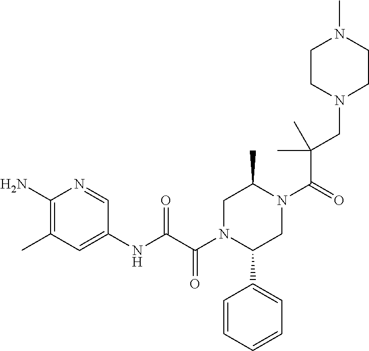 [SMILES: Cc1cc(NC(=O)C(=O)N2C[C@@H](C)N(C(=O)C(C)(C)CN3CCN(C)CC3)C[C@@H]2c2ccccc2)cnc1N] | 134 | (abs) | b | bb | ccc | C | A* |
| 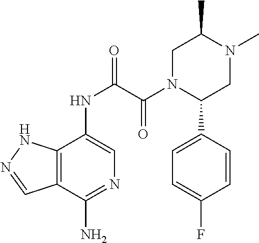 [SMILES: C[C@@H]1CN(C(=O)C(=O)Nc2cnc(N)c3cn[nH]c23)[C@@H](c2ccc(F)cc2)CN1C] | 135 | (abs) | c | aa | bbb | A | B* |
| 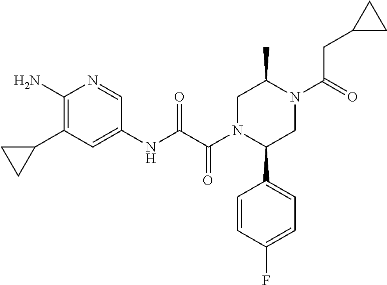 [SMILES: C[C@@H]1CN(C(=O)C(=O)Nc2cnc(N)c(C3CC3)c2)[C@H](c2ccc(F)cc2)CN1C(=O)CC1CC1] | 136 | (or) | | | | | C* |
| 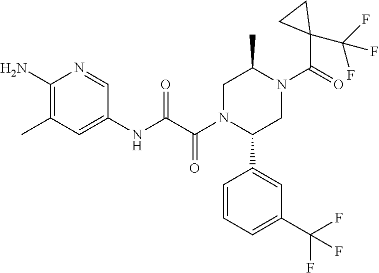 | 137 | (abs) | c | bb | ccc | B | A* |
| 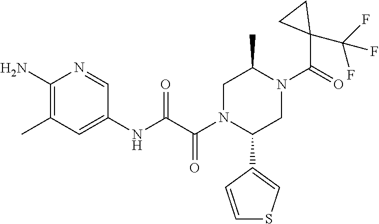 | 138 | (abs) | c | bb | ccc | A | A* |
| 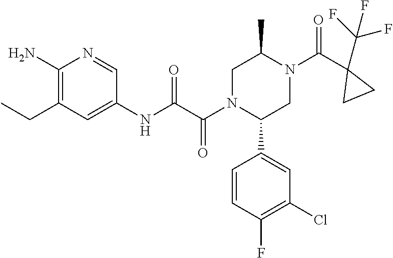 | 139 | (abs) | b | aa | aaa | B | A* |
| 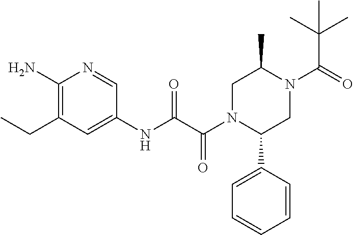 [SMILES: CCc1cc(NC(=O)C(=O)N2C[C@@H](C)N(C(=O)C(C)(C)C)C[C@@H]2c2ccccc2)cnc1N] | 140 | (abs) | b | aa | aaa | C | A* |
| 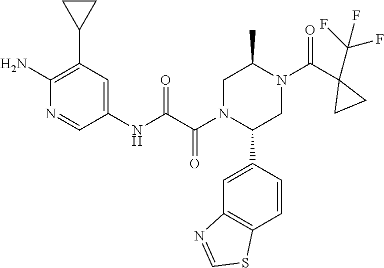 [SMILES: C[C@@H]1CN(C(=O)C(=O)Nc2cnc(N)c(C3CC3)c2)[C@@H](c2ccc3scnc3c2)CN1C(=O)C1(C(F)(F)F)CC1] | 141 | (abs) | b | aa | aaa | C | A* |
| 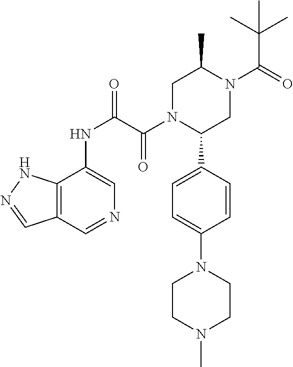 [SMILES: C[C@@H]1CN(C(=O)C(=O)Nc2cncc3cn[nH]c23)[C@@H](c2ccc(N3CCN(C)CC3)cc2)CN1C(=O)C(C)(C)C] | 142 | (abs) | c | bb | aaa | C | A* |
| 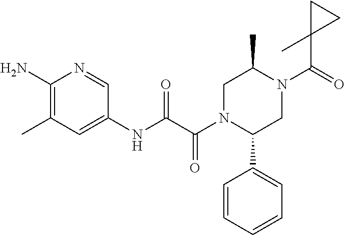 | 143 | (abs) | c | bb | ccc | C | A* |
| 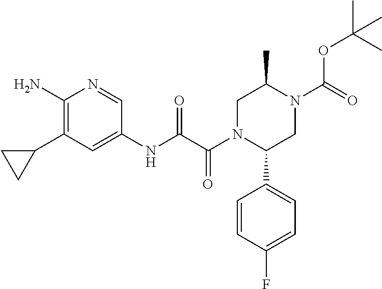 [SMILES: C[C@@H]1CN(C(=O)C(=O)Nc2cnc(N)c(C3CC3)c2)[C@@H](c2ccc(F)cc2)CN1C(=O)OC(C)(C)C] | 144 | (or) | c | aa | aaa | B | A* |
| 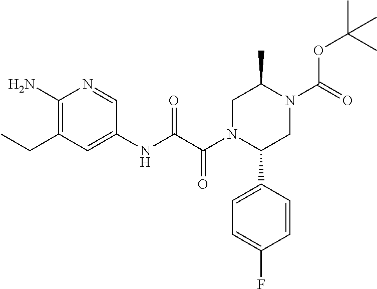 [SMILES: CCc1cc(NC(=O)C(=O)N2C[C@@H](C)N(C(=O)OC(C)(C)C)C[C@@H]2c2ccc(F)cc2)cnc1N] | 145 | (abs) | c | bb | aaa | B | A* |
| 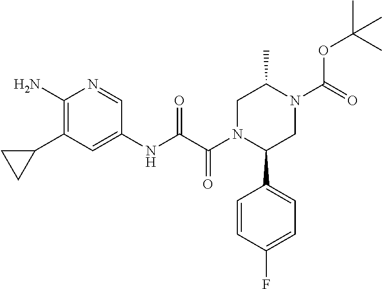 [SMILES: C[C@H]1CN(C(=O)C(=O)Nc2cnc(N)c(C3CC3)c2)[C@H](c2ccc(F)cc2)CN1C(=O)OC(C)(C)C] | 146 | (or) | c | cc | ccc | C | C* |
| 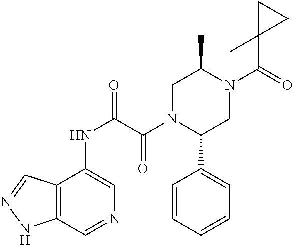 [SMILES: C[C@@H]1CN(C(=O)C(=O)Nc2cncc3[nH]ncc23)[C@@H](c2ccccc2)CN1C(=O)C1(C)CC1] | 147 | (abs) | c | aa | ccc | A | B* |
| 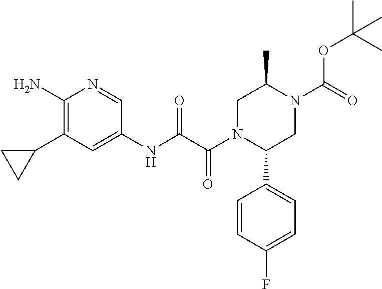 [SMILES: C[C@@H]1CN(C(=O)C(=O)Nc2cnc(N)c(C3CC3)c2)[C@@H](c2ccc(F)cc2)CN1C(=O)OC(C)(C)C] | 148 | (abs) | c | aa | ccc | A | A* |
| 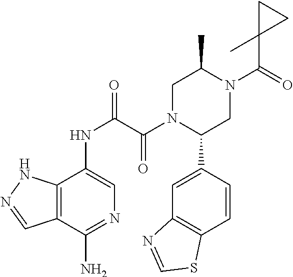 [SMILES: C[C@@H]1CN(C(=O)C(=O)Nc2cnc(N)c3cn[nH]c23)[C@@H](c2ccc3scnc3c2)CN1C(=O)C1(C)CC1] | 149 | (abs) | | | | | |
| 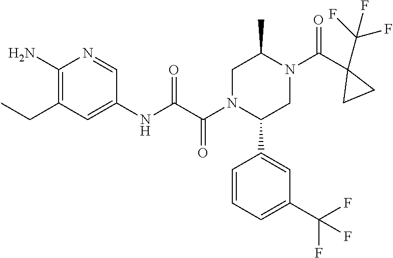 | 150 | (abs) | c | bb | ccc | A | A* |
| 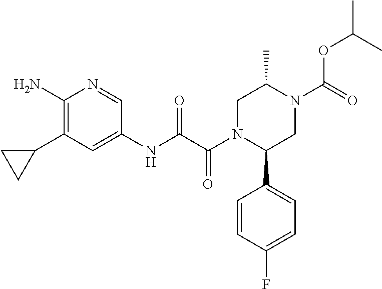 [SMILES: CC(C)OC(=O)N1C[C@@H](c2ccc(F)cc2)N(C(=O)C(=O)Nc2cnc(N)c(C3CC3)c2)C[C@@H]1C] | 151 | (or) | c | aa | aaa | B | A* |
| 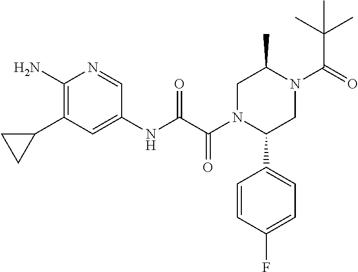 [SMILES: C[C@@H]1CN(C(=O)C(=O)Nc2cnc(N)c(C3CC3)c2)[C@@H](c2ccc(F)cc2)CN1C(=O)C(C)(C)C] | 152 | (abs) | b | aa | bbb | C | A* |
| 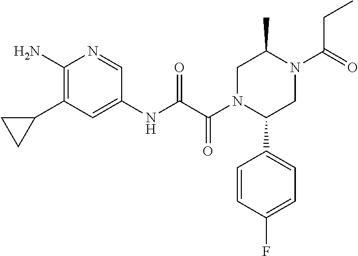 [SMILES: CCC(=O)N1C[C@H](c2ccc(F)cc2)N(C(=O)C(=O)Nc2cnc(N)c(C3CC3)c2)C[C@H]1C] | 153 | (abs) | c | bb | ccc | B | B* |
| 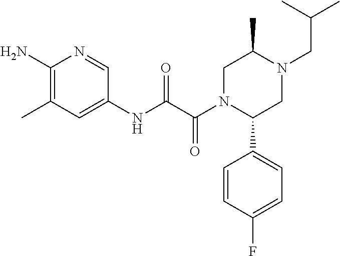 [SMILES: Cc1cc(NC(=O)C(=O)N2C[C@@H](C)N(CC(C)C)C[C@@H]2c2ccc(F)cc2)cnc1N] | 154 | (or) | c | cc | ccc | C | C* |
| 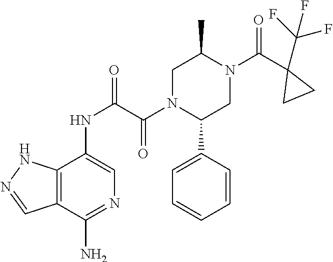 [SMILES: C[C@@H]1CN(C(=O)C(=O)Nc2cnc(N)c3cn[nH]c23)[C@@H](c2ccccc2)CN1C(=O)C1(C(F)(F)F)CC1] | 155 | (abs) | b | aa | aaa | B | A* |
| 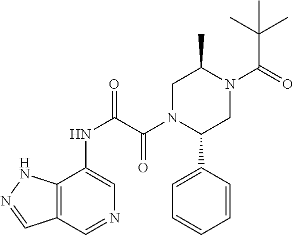 [SMILES: C[C@@H]1CN(C(=O)C(=O)Nc2cncc3cn[nH]c23)[C@@H](c2ccccc2)CN1C(=O)C(C)(C)C] | 156 | (abs) | b | aa | aaa | C | A* |
| 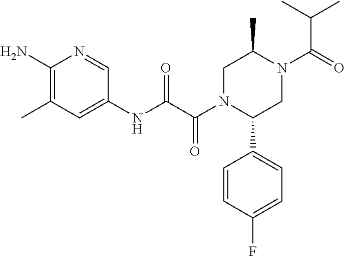 [SMILES: Cc1cc(NC(=O)C(=O)N2C[C@@H](C)N(C(=O)C(C)C)C[C@@H]2c2ccc(F)cc2)cnc1N] | 157 | (abs) | c | bb | ccc | C | B* |
| 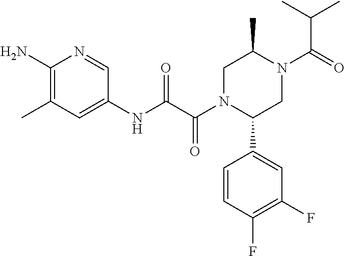 [SMILES: Cc1cc(NC(=O)C(=O)N2C[C@@H](C)N(C(=O)C(C)C)C[C@@H]2c2ccc(F)c(F)c2)cnc1N] | 158 | (abs) | c | bb | ccc | A | B* |
| 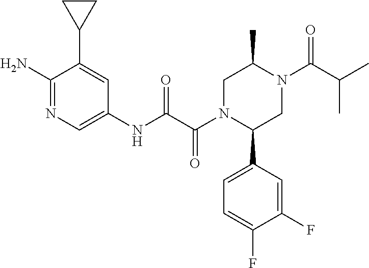 [SMILES: CC(C)C(=O)N1C[C@@H](c2ccc(F)c(F)c2)N(C(=O)C(=O)Nc2cnc(N)c(C3CC3)c2)C[C@H]1C] | 160 | (abs) | c | cc | ccc | C | C* |
| 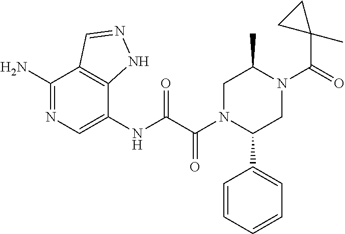 [SMILES: C[C@@H]1CN(C(=O)C(=O)Nc2cnc(N)c3cn[nH]c23)[C@@H](c2ccccc2)CN1C(=O)C1(C)CC1] | 161 | (abs) | b | aa | | C | A* |
| 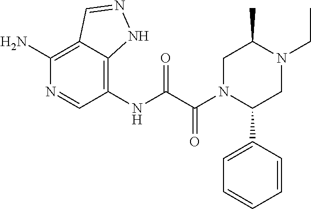 [SMILES: CCN1C[C@H](c2ccccc2)N(C(=O)C(=O)Nc2cnc(N)c3cn[nH]c23)C[C@H]1C] | 162 | (abs) | c | aa | | A | A* |
| 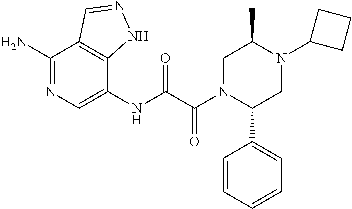 [SMILES: C[C@@H]1CN(C(=O)C(=O)Nc2cnc(N)c3cn[nH]c23)[C@@H](c2ccccc2)CN1C1CCC1] | 163 | (abs) | b | aa | | C | A* |
| 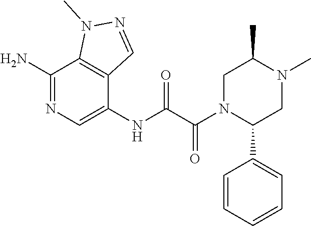 [SMILES: C[C@@H]1CN(C(=O)C(=O)Nc2cnc(N)c3c2cnn3C)[C@@H](c2ccccc2)CN1C] | 164 | (abs) | c | bb | bbb | A | A* |
| 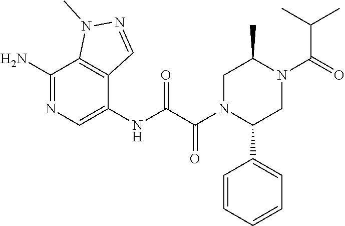 [SMILES: CC(C)C(=O)N1C[C@H](c2ccccc2)N(C(=O)C(=O)Nc2cnc(N)c3c2cnn3C)C[C@H]1C] | 165 | (abs) | b | bb | bbb | C | A* |
| 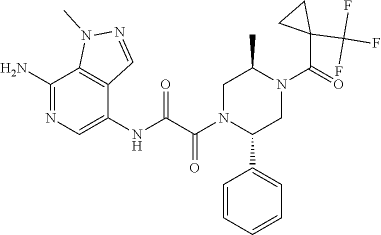 [SMILES: C[C@@H]1CN(C(=O)C(=O)Nc2cnc(N)c3c2cnn3C)[C@@H](c2ccccc2)CN1C(=O)C1(C(F)(F)F)CC1] | 166 | (abs) | b | aa | aaa | C | A* |
| 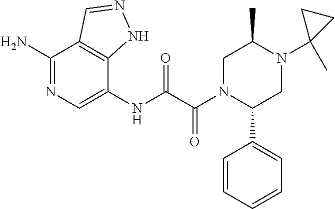 [SMILES: C[C@@H]1CN(C(=O)C(=O)Nc2cnc(N)c3cn[nH]c23)[C@@H](c2ccccc2)CN1C1(C)CC1] | 167 | (abs) | c | aa | | A | A* |
| 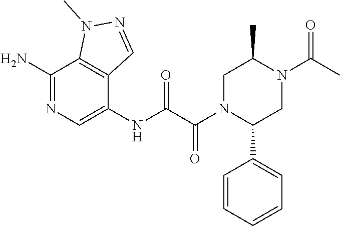 [SMILES: CC(=O)N1C[C@H](c2ccccc2)N(C(=O)C(=O)Nc2cnc(N)c3c2cnn3C)C[C@H]1C] | 168 | (abs) | | | | | A* |
| 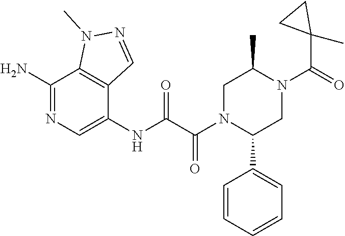 [SMILES: C[C@@H]1CN(C(=O)C(=O)Nc2cnc(N)c3c2cnn3C)[C@@H](c2ccccc2)CN1C(=O)C1(C)CC1] | 169 | (abs) | b | aa | aaa | C | A* |
| 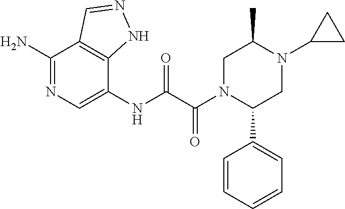 [SMILES: C[C@@H]1CN(C(=O)C(=O)Nc2cnc(N)c3cn[nH]c23)[C@@H](c2ccccc2)CN1C1CC1] | 170 | (abs) | b | aa | | C | A* |

### Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, 180; nitrogen may be, for example, ¹⁵N, and the like. In other embodiments, a particular isotope (e.g., ³H, ¹³C, ¹⁴C, ¹⁸O, or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In another embodiment, provided is a pharmaceutical composition comprising a pharmaceutically acceptable carrier and an effective amount of a compound described herein (e.g., a compound of Formula (I) or a compound of Table 1), or a pharmaceutically acceptable salt thereof.

The term “pharmaceutically acceptable carrier or adjuvant” refers to a carrier or adjuvant that may be administered to a patient, together with a compound provided herewith, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions provided herewith include, but are not limited to, ion exchangers, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose based substances, polyethylene glycol, sodium carboxymethylcellulose, polyacrylates, waxes, polyethylene polyoxypropylene block polymers, polyethylene glycol and wool fat. Cyclodextrins such as a-, 0-, and y-cyclodextrin, or chemically modified derivatives such as hydroxyalkylcyclodextrins, including 2 and 3 hydroxypropyl-o-cyclodextrins, or other solubilized derivatives may also be advantageously used to enhance delivery of compounds of the formulae described herein.

When employed as pharmaceuticals, the compounds provided herein are typically administered in the form of a pharmaceutical composition. Such compositions can be prepared in a manner well known in the pharmaceutical art and comprise at least one active compound.

In one embodiment, with respect to the pharmaceutical composition, the carrier is a parenteral carrier, oral or topical carrier.

Also provided is a compound described herein (e.g., a compound of Formula (I) or a compound of Table 1, or pharmaceutically acceptable salts thereof) (or pharmaceutical composition thereof) for use as a pharmaceutical or a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (I) or a compound of Table 1, or pharmaceutically acceptable salts thereof) (or pharmaceutical composition thereof) for use in the treatment of an MTAP-deficient and/or an MTA-accumulating disease in a subject in need thereof. In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Also provided is a compound described herein (e.g., a compound of Formula (I) or a compound of Table 1, or pharmaceutically acceptable salts thereof) (or pharmaceutical composition thereof) for use in the manufacturing of a medicament (e.g., a medicament for the treatment of an MTAP-deficient and/or an MTA-accumulating disease in a subject in need thereof). In one embodiment, the disease is a proliferating disease. In a further embodiment, the disease is an MTAP-deficient and/or MTA-accumulating cancer. In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Generally, the compounds provided herein are administered in an effective amount (e.g., a therapeutically effective amount). The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions provided herewith may be administered orally, parenterally, by inhalation spray, topically, rectally, nasally, buccally, vaginally or via an implanted reservoir, preferably by oral administration or administration by injection. The pharmaceutical compositions provided herewith may contain any conventional nontoxic pharmaceutically acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrastenal, intrathecal, intralesional and intracranial injection or infusion techniques.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like. The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as an ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The pharmaceutical compositions provided herewith may also be administered in the form of suppositories for rectal administration. These compositions can be prepared by mixing a compound provided herewith with a suitable non irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions provided herewith may be administered by nasal aerosol or inhalation. Such compositions are prepared according to techniques well known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The above-described components for orally administrable, injectable or topically administrable, rectally administrable and nasally administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds described herein can also be administered in sustained release forms or from sustained release drug delivery systems. A description of representative sustained release materials can be found in Remington's Pharmaceutical Sciences.

When the compositions provided herewith comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds provided herewith. Alternatively, those agents may be part of a single dosage form, mixed together with the compounds provided herewith in a single composition.

Also provided is the pharmaceutically acceptable acid addition salt of a compound described herein (e.g., compound of Formula (I) or a compound of Table 1).

The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions provided herewith will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination provided herewith may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long term basis upon any recurrence of disease symptoms.

### Methods of Treatment and Use

Treatment of MTAP-Deficient and/or MTA-Accumulating Proliferation Disorders

5-Methylthioadenosine phosphorylase (MTAP) catalyzes the reversible phosphorylation of S-methyl-5′-thioadenosine (MTA) to adenine and 5-methylthioribose-1-phosphate. MTAP-deletion is a common genetic event in human cancer. MTAP deletion frequency in a subset of human cancers is described in Cerami et al., Cancer Discov. (2012); 2(5):401-4; Gao et al., Sci Signal. (2013); 6(269):pl1; and Lee et al., Nat. Gen. (2014) 46(11):1227-32. For example, more than 50% of malignant peripheral nerve sheath tumor (MPNST) have deletions in MTAP (Lee et al., Nat. Gen. (2014)). Other cancers with high MTAP deletion frequencies are glioblastoma (GBM), mesothelioma, bladder cancer, pancreatic cancer, esophageal cancer, squamous lung cancer, melanoma, diffuse large B cell lymphoma (DLBCL), head and neck cancer, cholangiocarcinoma, lung adenoma, sarcoma, stomach cancer, glioma, adrenal carcinoma, thymoma, breast cancer, liver cancer, ovarian cancer, renal papillary cancer, uterine cancer, prostate cancer, and renal clear cell cancer. MTAP deletion in cells is one of the mechanisms that leads to MTAP-deficiency, increased intracellular MTA accumulation, and confers enhanced dependency on the protein arginine methyltransferase 5 (PRMT5) in cancer cells. Other mechanisms leading to MTAP deficiency include, inter alia, MTAP translocations and MTAP epigenetic silencing which could also lead to MTAP-null and/or MTAP deficient tumors. PRMT5 mediates the formation of symmetric dimethylarginine (SDMA); thus, the PRMT5 activity can be assessed by measuring the SDMA levels using the antibody against an SDMA or SDMA modified polypeptide.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of a compound of the present disclosure (e.g., a compound of Formula (I) or a compound of Table 1) or a pharmaceutically acceptable salt thereof.

In some embodiments, provided is a compound of the present disclosure (e.g., a compound of Formula (I) or a compound of Table 1), or a pharmaceutical composition comprising a compound of Formula (I) of the present disclosure for use in a method of treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a compound of the present disclosure (e.g., a compound of Formula (I) or a compound of Table 1), or a pharmaceutical composition comprising a compound of Formula (I) of the present disclosure for use in the manufacturing of a medicament for treating human or animal subjects having or having been diagnosed with an MTAP-deficiency-related and/or MTA-accumulating proliferative disorder (e.g., cancer). In some embodiments, the compound or composition is provided in a therapeutically effective amount.

In some embodiments, provided is a use of a compound of the present disclosure (e.g., a compound of Formula (I) or a compound of Table 1), or of a pharmaceutical composition comprising a compound of Formula (I) of the present disclosure in a method of treating human or animal subjects having or having been diagnosed with an MTAP-deficiency-related and/or MTA-accumulating proliferative disorder (e.g., cancer). In some embodiments, the use is of a therapeutically effective amount of the compound or composition.

In some embodiments, provided is use of a compound of the present disclosure (e.g., a compound of Formula (I) or a compound of Table 1), or of a pharmaceutical composition comprising a compound of Formula (I) of the present disclosure in the manufacturing of a medicament for treating human or animal subjects having or having been diagnosed with an MTAP-deficiency-related and/or MTA-accumulating proliferative disorder (e.g., cancer). In some embodiments, the use is of a therapeutically effective amount of the compound or composition.

In one embodiment, provided are methods for treating an MTAP-deficiency-related and/or MTA-accumulating proliferative disorder (e.g., cancer) in a subject in need thereof comprising administering to the subject an effective amount (e.g., a therapeutically effective amount) of a compound of the present disclosure (e.g., compound of Formula (I) or a compound of Table 1) or a pharmaceutically acceptable salt thereof.

In one embodiment, provided are methods of treating human or animal subjects having or having been diagnosed with an MTAP-deficiency-related and/or MTA-accumulating proliferative disorder (e.g., cancer) comprising administering to the subject in need thereof a therapeutically effective amount of pharmaceutical composition of the present disclosure (e.g., a composition comprising a compound of Formula (I) or a compound of Table 1, or pharmaceutically acceptable salts thereof and a pharmaceutically acceptable carrier). In one embodiment, the compound or composition is administered in combination with a second therapeutic agent.

In one embodiment, provided are methods of treating an MTAP-deficiency-related and/or MTA-accumulating proliferative disorder (e.g., cancer) in a subject in need thereof comprising administering to the subject a therapeutically effective amount of pharmaceutical composition of the present disclosure (e.g., a composition comprising a compound of Formula (I) or a compound of Table 1, or pharmaceutically acceptable salts thereof and a pharmaceutically acceptable carrier). In one embodiment, the compound or composition is administered in combination with a second therapeutic agent.

In some embodiments, the subject is human.

In certain embodiments, the disease is an MTAP-deficient and/or MTA-accumulating cancer.

In one embodiment, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, the cancer is an MTAP-deficient and/or MTA-accumulating glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (I) or a compound of Table 1, or pharmaceutically acceptable salts thereof) described herein can be used in a method of inhibiting proliferation of MTAP-deficient cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (I) or a compound of Table 1, or pharmaceutically acceptable salts thereof) in an amount that is effective to inhibit proliferation of the MTAP-deficient cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive or mixed mode PRMT5 inhibitor or an MTA cooperative binding agent, e.g., a compound of Formula (I) or a compound of Table 1, or pharmaceutically acceptable salts thereof) described herein can be used in a method of inhibiting proliferation of MTA-accumulating cells in a subject in need thereof, the method comprising the step of administering to the subject, a PRMT5 inhibitor (e.g., an MTA-uncompetitive, non-competitive or mixed mode PRMT5 inhibitor or an MTA cooperative binding agent, e.g., a compound of Formula (I) or a compound of Table 1, or pharmaceutically acceptable salts thereof) in an amount that is effective to inhibit proliferation of the MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

The PRMT5 inhibitors (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) described herein can be used in a method of inhibiting proliferation of MTAP deficient and/or MTA-accumulating cells in a subject in need thereof, the method comprising the step of administering to the subject, a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) in an amount that is effective to inhibit proliferation of the MTAP deficient and/or MTA-accumulating cells. In one embodiment, the subject in need thereof suffers from a cancer selected from the group consisting of glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

### Combination Therapies

In some embodiments, provided are methods of treatment of MTAP-deficient and/or MTA accumulating proliferative disorders (e.g., cancers) with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) in combination with one or more therapeutic agent.

In some embodiments, provided are methods of treatment of MTAP-deficient and/or MTA accumulating proliferative disorders (e.g., cancers) with a PRMT5 inhibitor described herein (e.g., a compound of Formula (I) or a compound of Table 1, or pharmaceutically acceptable salts thereof) in combination with a second therapeutic agent. In some embodiments, provided are methods of treatment of MTAP-deficient and/or MTA accumulating proliferative disorders (e.g., cancers) with PRMT5 inhibitor described herein (e.g., a compound of Formula (I) or a compound of Table 1, or pharmaceutically acceptable salts thereof) in combination with a second therapeutic agent and a third therapeutic agent. In some embodiments, provided are methods of treatment of MTAP-deficient and/or MTA accumulating proliferative disorders (e.g., cancers) with PRMT5 inhibitor described herein (e.g., a compound of Formula (I) or a compound of Table 1, or pharmaceutically acceptable salts thereof) in combination with a second therapeutic agent, a third therapeutic agent, and a fourth therapeutic agent.

The term “Combination” refers to either a fixed combination in one dosage unit form, or a combined administration where a PRMT5 inhibitor described herein (e.g., a compound of Formula (I) or a compound of Table 1, or pharmaceutically acceptable salts thereof) and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g., PRMT5 inhibitors described herein (e.g., a compound of Formula (I) or a compound of Table 1, or pharmaceutically acceptable salts thereof) and a combination partner, are both administered to a patient simultaneously in the form of a single entity or dosage. The term “non-fixed combination” means that the therapeutic agents, e.g., a PRMT5 inhibitor described herein (e.g., a compound of Formula (I) or a compound of Table 1, or pharmaceutically acceptable salts thereof) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more therapeutic agent.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., tablets, capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times.

In certain embodiments, PRMT5 inhibitors described herein are combined with other therapeutic agents, including, but not limited to, other anti-cancer agents, anti-allergic agents, anti-nausea agents (or anti-emetics), pain relievers, cytoprotective agents, and combinations thereof.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a general chemotherapeutic agents selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an EGFR-inhibitor (e.g., cetuximab, panitumimab, erlotinib, gefitinib and EGFRi NOS). In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a MAPK-pathway inhibitor (e.g., BRAFi, panRAFi, MEKi, ERKi). In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a PI3K-mTOR pathway inhibitor (e.g., alpha-specific PI3Ki, pan-class I PI3Ki and mTOR/PI3Ki, particularly everolimus and analogues thereof).

MTAP-deletion can co-occur with mutations in the KRAS gene (e.g., KRASG12C). In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof), and a KRAS inhibitor (e.g., a pan-KRAS or a specific G12C, G12D, G13C inhibitor, e.g., adagrasib, sotorasib, LY3537982, RMC-6236, RMC-6291, RMC-9805, RMC-8839).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof), and a Spliceosome inhibitor (e.g., SF3b1 inhibitors; e.g., E7107).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an HDAC inhibitor or DNA methyltransferase inhibitor. In some embodiments, the HDAC inhibitor is Trichostatin A. In some embodiments, the DNA methyltransferase inhibitor is 5-azacytidine.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a MAT2A inhibitor.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an inhibitor of a protein which interacts with or is required for PRMT5 function, including, but not limited to, pICIN, WDR77 or RIOK1.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an HDM2 inhibitor and/or 5-FU or other purine analogues (e.g., 6-thioguanine, 6-mercaptopurine).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a CDK4 inhibitor, including, but not limited to, LEE011 or a CDK 4/6 inhibitor (e.g., palbociclib (Ibrance®), ribociclib (Kisqali®), and abemaciclib (Verzenio®).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a targeted treatment contingent on the dependency of individual target tumors on relevant pathways as determined by suitable predictive markers, including but not limited to: inhibitors of HDM2i, PI3K/mTOR-I, MAPKi, RTKi (EGFRi, FGFRi, METi, IGFiRi, JAKi, and WNTi.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and immunotherapy.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an immunotherapeutic agent.

In some embodiments, the immunotherapeutic agent is an anti-CTLA-4 antibody (e.g., ipilimumab, tremelimumab).

In some embodiments, the immunotherapeutic agent is an anti-PD-1 or anti-PD-L1 agent (e.g., an antibody). In some embodiments, the immunotherapeutic agent is an anti-PD-1 agent (e.g., an anti-PD-1 antibody, e.g., nivolumab (i.e., MDX-1106, BMS-936558, ONO-4538); CT-011; AMP-224; pembrolizumab (MK-3475); pidilizumab; cemiplimab; dostarlimab; prolgolimab; spartalizumab; camrelizumab; sasanlimab, sintilimab; tislelizumab; toripalimab; retifanlimab; MEDI0680; budigalimab; geptanolimab). In some embodiments, the immunotherapeutic agent is an anti-PD-L1 agent (e.g., an anti-PD-L1 antibody, e.g., BMS936559 (i.e., MDX-1105); durvalumab (MED14736); avelumab (MSB0010718C); envafolimab; cosibelimab; sugemalimab, AUNP-12 or atezolizumab (MPDL-3280A) or an anti-PD-L1 small molecule (e.g., CA-170)).

In some embodiments, the immunotherapeutic agent is a checkpoint blocking antibody (e.g., anti-TIM3, anti-LAG3, anti-TIGIT including IMP321 and MGA271).

In some embodiments, the immunotherapeutic agent is a cell-based therapy. In some embodiments, the cell-based therapy is a CAR-T therapy.

In some embodiments, the immunotherapeutic agent is a co-stimulatory antibody (e.g., anti-4-1BB, anti-OX40, anti-GITR, anti-CD27, anti-CD40).

In some embodiments, the immunotherapeutic agent is a cancer vaccine such as a neoantigen. These vaccines can be developed using peptides or RNA (e.g., mRNA).

In some embodiments, the immunotherapeutic agent is an oncolytic virus.

In some embodiments, the immunotherapeutic agent is a STING pathway agonist. Exemplary STING agonists include MK-1454 and ADU-S100.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a disease-specific huMAB (e.g., an anti-HER3 huMAB).

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an ADC/ADCC contingent on the expression of relevant surface targets on target tumors of interest.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and one or more DNA damage pathway inhibitor. In some embodiments, a DNA damage pathway inhibitor is selected from the group consisting of bleomycin, an ATM inhibitor (e.g., AZD1390), a USP1 inhibitor, a WEEl inhibitor (e.g., AZD1775), and a Chk1 inhibitor (e.g., AZD7762). In some embodiments, a DNA damage pathway inhibitor is a DNA alkylating agent.

In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a PARP inhibitor. In some embodiments, a PARP inhibitor is selected from the group consisting of olaparib, rucaparib, niraparib, talazoparib, veliparib, pamiparib, CEP 9722, E7016, iniparib, and 3-aminobenzamide.

Some patients may experience allergic reactions to the PRMT5 inhibitors described herein and/or other anti-cancer agent(s) during or after administration; therefore, anti-allergic agents are often administered to minimize the risk of an allergic reaction. In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an anti-allergic agent (e.g., a corticosteroid, including, but not limited to, dexamethasone (e.g., Decadron®), beclomethasone (e.g., Beclovent®), hydrocortisone (also known as cortisone, hydrocortisone sodium succinate, hydrocortisone sodium phosphate, and sold under the tradenames Ala-Cort®, hydrocortisone phosphate, Solu-Cortef®, Hydrocort Acetate® and Lanacort®), prednisolone (sold under the tradenames Delta-Cortel®, Orapred®, Pediapred® and Prelone®), prednisone (sold under the tradenames Deltasone®, Liquid Red®, Meticorten® and Orasone®), methylprednisolone (also known as 6-methylprednisolone, methylprednisolone acetate, methylprednisolone sodium succinate, sold under the tradenames Duralone®, Medralone®, Medrol®, M—Prednisol® and Solu-Medrol®); an antihistamine, such as diphenhydramine (e.g., Benadryl®), hydroxyzine, and cyproheptadine; a bronchodilator, such as the beta-adrenergic receptor agonists, albuterol (e.g., Proventil®), and terbutaline (Brethine®)).

Some patients may experience nausea during and after administration of the PRMT5 inhibitors described herein and/or other anti-cancer agent(s); therefore, anti-emetics are used in preventing nausea (upper stomach) and vomiting. In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an anti-emetic (e.g., aprepitant (Emend®), ondansetron (Zofran®), granisetron HCl (Kytril®), lorazepam (Ativan®), dexamethasone (Decadron®), prochlorperazine (Compazine®), casopitant (Rezonic® and Zunrisa®), and combinations thereof).

Medication to alleviate the pain experienced during the treatment period is often prescribed to make the patient more comfortable. In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and an analgesic (e.g., an over-the-counter analgesic (e.g., Tylenol®), an opioid analgesic (e.g., hydrocodone/paracetamol or hydrocodone/acetaminophen (e.g., Vicodin®), morphine (e.g., Astramorph® or Avinza®), oxycodone (e.g., OxyContin® or Percocet®), oxymorphone hydrochloride (Opana®), fentanyl (e.g., Duragesic®))).

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy. In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and a cytoprotective agent (e.g., Amifostine (Ethyol®), glutamine, dimesna (Tavocept®), mesna (Mesnex®), dexrazoxane (Zinecard® or Totect®), xaliproden (Xaprila®), and leucovorin (also known as calcium leucovorin, citrovorum factor and folinic acid)).

The structure of the active compounds identified by code numbers, generic or trade names may be taken from the actual edition of the standard compendium “The Merck Index” or from databases, e.g., Patents International (e.g., IMS World Publications).

The above-mentioned compounds, which can be used in combination with a PRMT5 inhibitor as described herein, can be prepared and administered as described in the art, including, but not limited to, in the documents cited above.

In one embodiment, provided are pharmaceutical compositions comprising at least one compound of the present disclosure (e.g., a PRMT5 inhibitor, e.g., a compound of Formula (I) or a compound of Table 1) or a pharmaceutically acceptable salt thereof together with a pharmaceutically acceptable carrier suitable for administration to a human or animal subject, either alone or together with other anti-cancer agents.

In one embodiment, provided are methods of treating human or animal subjects having or having been diagnosed with an MTAP-deficient and/or MTA accumulating proliferative disorder (e.g., cancer) comprising administering to the subject in need thereof a pharmaceutical composition comprising a therapeutically effective amount of a compound of the present disclosure (e.g., a compound of Formula (I) or a compound of Table 1) or a pharmaceutically acceptable salt thereof in combination with one or more therapeutic agents as described herein.

In one embodiment, provided are methods of treating an MTAP-deficient and/or MTA accumulating proliferative disorder (e.g., cancer) in a subject in need thereof comprising administering to the subject a pharmaceutical composition comprising an effective amount (e.g., a therapeutically effective amount) of a compound of the present disclosure (e.g., a compound of Formula (I) or a compound of Table 1) or a pharmaceutically acceptable salt thereof in combination with one or more therapeutic agents as described herein.

In particular, compositions will either be formulated together as a combination therapeutic or administered separately.

In combination therapy, a PRMT5 inhibitor as described herein and other anti-cancer agent(s) may be administered either simultaneously, concurrently or sequentially with no specific time limits, wherein such administration provides therapeutically effective levels of the two compounds in the body of the patient.

In a preferred embodiment, the compound of the present disclosure (e.g., a compound of Formula (I) or a compound of Table 1, or pharmaceutically acceptable salts thereof) and the other anti-cancer agent(s) is generally administered sequentially in any order by infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The PRMT5 inhibitor as described herein and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In another embodiment, provided are kits that include one or more PRMT5 inhibitor(s) as described herein (e.g., a compound of Formula (I) or a compound of Table 1, or pharmaceutically acceptable salts thereof) and a second therapeutic agent as disclosed herein are provided. Representative kits include (a) a PRMT5 inhibitor as described herein or a pharmaceutically acceptable salt thereof (e.g., a compound of Formula (I) or a compound of Table 1, or pharmaceutically acceptable salts thereof), (b) at least one other therapeutic agent, e.g., as indicated above, whereby such kit may comprise a package insert or other labeling including directions for administration.

A PRMT5 inhibitor as described herein (e.g., a compound of Formula (I) or a compound of Table 1, or pharmaceutically acceptable salts thereof) may also be used in combination with known therapeutic processes, for example, the administration of hormones or especially radiation. A compound of the present disclosure may in particular be used as a radiosensitizer, especially for the treatment of tumors which exhibit poor sensitivity to radiotherapy. In some embodiments, provided is a method of treating a disease or disorder (e.g., cancer) comprising administering or coadministering, in any order, to a patient in need thereof a PRMT5 inhibitor described herein (e.g., a compound of Formula (I) or a compound of Table 1, or pharmaceutically acceptable salts thereof) and radiation.

### Patient Selection and Monitoring

In one embodiment, provided is a method of determining if a subject having or having been diagnosed with a cancer (e.g., a cancer patient) will respond to therapeutic treatment with a PRMT5 inhibitor (e.g., an MTA-uncompetitive PRMT5 inhibitor, e.g., a compound of Formula (I) or a compound of Table 1, or pharmaceutically acceptable salts thereof), comprising the steps of:
a) contacting a test sample obtained from said subject with a reagent capable of detecting human cancer cells that have MTAP deficiency and/or MTA accumulation; and
b) comparing the test sample with a reference (e.g., a reference sample taken from a non-cancerous or normal control subject),
wherein the presence of MTAP deficiency and/or MTA accumulation in said test sample indicates that the subject will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof).

In one embodiment, provided is a method of determining if a cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof), comprising the steps of:
a) contacting a test sample obtained from a subject having or having been diagnosed with said cancer with a reagent capable of detecting human cancer cells that have MTAP deficiency and/or MTA accumulation; and
b) comparing the test sample with a reference (e.g., a reference sample taken from a non-cancerous or normal control subject),
   wherein the presence of MTAP deficiency and/or MTA accumulation in said test sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof). In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma. In some embodiments, the method further comprises the step of determining the level of PRMT5 in the cancer cells. The level of expression of PRMT5 can be considered when determining the therapeutically effective dosage of a PRMT5 inhibitor.

In one embodiment, provided is a method of determining the sensitivity of a cancer cell to PRMT5 inhibition (e.g., inhibition with an MTA-uncompetitive PRMT5 inhibitor, e.g., a compound of Formula (I) or a compound of Table 1, or pharmaceutically acceptable salts thereof), comprising the steps of:
a) assaying the production, level, activity, expression or presence of MTAP), in said cancer cell;
b) comparing the production, level, activity, expression or presence of MTAP in the cancer cell with the production, level, activity, expression or presence of MTAP, respectively, in a non-cancerous or normal control cell, wherein a decreased level, activity or expression in the cancer cell indicates MTAP deficiency and wherein MTAP deficiency indicates that said cancer cell is sensitive to the PRMT5 inhibitor.
   In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment, provided is a method of determining the sensitivity of a cancer cell to a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof), comprising the steps of:
a) assaying for level, activity or expression of the MTAP gene or its gene product in both the cancer cell and a normal control cell, wherein a decreased level, activity or expression in the cancer cell indicates MTAP deficiency; b) assaying for PRMT5 expression in said cancer cell; c) comparing the PRMT5 expression with PRMT5 expression in the cancer cell and a normal control cell; wherein the similarity in PRMT5 expression, and the presence of said MTAP deficiency in said cancer cell, indicates said cell is sensitive to a PRMT5 inhibitor.

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In one embodiment the provided is a therapeutic method of treating a subject having or having been diagnosed with a cancer (e.g., a cancer associated with MTAP deficiency and/or MTA accumulation) comprising the steps of:
a) assessing the level of MTAP and/or MTA in a test sample obtained from said subject (e.g., by contacting the sample with a reagent capable of detecting human MTAP-deficient and/or MTA-accumulating cancer cells in a test sample obtained from said subject), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor; and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) to the subject identified in step b).

In one embodiment provided is a therapeutic method of treating a cancer (e.g., a cancer associated with MTAP deficiency and/or MTA accumulation) in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a therapeutic method of treating a subject having or having been diagnosed with a cancer associated with MTAP deficiency and/or MTA accumulation comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) to the subject identified in step b).

In one embodiment provided is a therapeutic method of treating cancer associated with MTAP deficiency and/or MTA accumulation in a subject in need thereof comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference sample (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent); and
c) administering a therapeutically effective amount of a composition comprising a PRMT5 inhibitor (e.g., an MTA-uncompetitive PRMT5 inhibitor e.g., a compound of Formula (I) or a compound of Table 1, or pharmaceutically acceptable salts thereof) to the subject identified in step b).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

In one embodiment provided is a method of determining if a subject having or having been diagnosed with a cancer associated with MTAP deficiency and/or MTA accumulation will respond to treatment with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) comprising the steps of:
a) assessing the level of MTAP and/or MTA in a test sample obtained from said subject (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said subject will respond to therapeutic treatment with a PRMT5 inhibitor (e.g., an MTA-uncompetitive, non-competitive, or mixed mode PRMT5 inhibitor or an MTA-cooperative binding agent).

In one embodiment provided is a method of determining if a cancer associated with MTAP deficiency and/or MTA accumulation will respond to treatment with a PRMT5 inhibitor (e.g., an MTA-uncompetitive PRMT5 inhibitor, e.g., a compound of Formula (I) or a compound of Table 1, or pharmaceutically acceptable salts thereof) comprising the steps of:
a) assessing the level of MTAP and/or MTA in a test sample obtained from a subject having or having been diagnosed with said cancer (e.g., by contacting the sample with a reagent capable of detecting human MTAP-deficient and/or MTA-accumulating cancer cells), wherein the MTA level can be assessed directly (e.g., by ELISA or LC-MS/MS) or indirectly (e.g., by SDMA-modified protein ELISA or IHC, or by RNA splicing);
b) comparing the test sample with a reference (e.g., a reference sample taken from a non-cancerous or normal control subject), wherein MTAP deficiency and/or MTA accumulation in said test sample indicates said cancer will respond to therapeutic treatment with a PRMT5 inhibitor (e.g., an MTA-uncompetitive, non-competitive, or mixed mode PRMT5 inhibitor or an MTA-cooperative binding agent).

In some embodiments, the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In some embodiments, the method further comprises the step of determining the level of PRMT5 in the cancer cells.

### Sample Preparation

Further provided are assays for the detection of MTAP deficiency and/or MTA accumulation. They can include detecting a mutation related to MTAP deficiency and/or MTA accumulation, e.g., in a body fluid such as blood (e.g., serum or plasma) bone marrow, cerebral spinal fluid, peritoneal/pleural fluid, lymph fluid, ascites, serous fluid, sputum, lacrimal fluid, stool, and urine, or in a tissue such as a tumor tissue. The tumor tissue can be fresh tissue or preserved tissue (e.g., formalin fixed tissue, e.g., paraffin-embedded tissue).

Body fluid samples can be obtained from a subject using any of the methods known in the art. Methods for extracting cellular DNA from body fluid samples are well known in the art. Typically, cells are lysed with detergents. After cell lysis, proteins are removed from DNA using various proteases. DNA is then extracted with phenol, precipitated in alcohol, and dissolved in an aqueous solution. Methods for extracting acellular DNA from body fluid samples are also known in the art. Commonly, a cellular DNA in a body fluid sample is separated from cells, precipitated in alcohol, and dissolved in an aqueous solution.

### Detection of PRMT5 Selectivity

Samples, once prepared, can be tested for MTAP deficiency and/or MTA accumulation, either or both of which indicates that the sample is sensitive to treatment with a PRMT5 inhibitor. Cells can be determined to be MTA accumulating by techniques known in the art; methods for detecting MTA include, as a non-limiting example, liquid chromatography-electrospray ionization-tandem mass spectrometry (LC-ESI-MS/MS), as described in Stevens et al. 2010. J. Chromatogr. A. 1217: 3282-3288; and Kirovski et al. 2011 Am. J. Pathol. 178: 1145-1152; and references cited therein. The detection of MTAP deficiency can be done by any number of ways, for example: DNA sequencing, PCR based methods, including RT-PCR, microarray analysis, Southern blotting, Northern blotting, Next Generation Sequencing, and dip stick analysis. In some embodiments, MTAP deficiency is evaluated by any technique known in the art, for example, immunohistochemistry utilizing an anti-MTAP antibody or derivative thereof, and/or genomic sequencing, or nucleic acid hybridization, or amplification utilizing at least one probe or primer comprising a sequence of at least 12 contiguous nucleotides (nt) of the sequence of MTAP wherein the primer is no longer than about 30 nt.

The polymerase chain reaction (PCR) can be used to amplify and identify MTAP deficiency from either genomic DNA or RNA extracted from tumor tissue. PCR is well known in the art and is described in detail in Saiki et al., Science 1988, 239:487.

Methods of detecting MTAP deficiency by hybridization are provided. The method comprises identifying MTAP deficiency in a sample by its inability to hybridize to MTAP nucleic acid. The nucleic acid probe is detectably labeled with a label such as a radioisotope, a fluorescent agent or a chromogenic agent. Rddioisotopes can include without limitation; 3H, 32P, 33P and 35S etc. Fluorescent agents can include without limitation: FITC, texas red, rhodamine, etc.

The probe used in detection that is capable of hybridizing to MTAP nucleic acid can be from about 8 nucleotides to about 100 nucleotides, from about 10 nucleotides to about 75 nucleotides, from about 15 nucleotides to about 50 nucleotides, or about 20 to about 30 nucleotides. The kit can also provide instructions for analysis of patient cancer samples, wherein the presence or absence of MTAP deficiency indicates if the subject is sensitive or insensitive to treatment with a PRMT5 inhibitor.

Single stranded conformational polymorphism (SSCP) can also be used to detect MTAP deficiency. This technique is well described in Orita et al., PNAS 1989, 86:2766-2770.

### Measurement of Gene Expression

Evaluation of MTAP deficiency and measurement of MTAP gene expression, and measurement of PRMT5 gene expression can be performed using any method or reagent known in the art.

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one embodiment, gene expression is detected and quantitated by hybridization to a probe that specifically hybridizes to the appropriate probe for that biomarker. The probes also can be attached to a solid support for use in high throughput screening assays using methods known in the art.

In one embodiment, the expression level of a gene is determined through exposure of a nucleic acid sample to the probe-modified chip. Extracted nucleic acid is labeled, for example, with a fluorescent tag, preferably during an amplification step.

Hybridization of the labeled sample is performed at an appropriate stringency level. The degree of probe-nucleic acid hybridization is quantitatively measured using a detection device.

Alternatively, any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one embodiment, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g., fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g., with a labeled RNA) by kinasing of the nucleic acid and subsequent attachment (ligation) of a nucleic acid linker joining the sample nucleic acid to a label (e.g., a fluorophore).

In one example, the gene expression can be measured through an in-situ hybridization protocol that can detect RNA molecules on a slide containing tissue sections or cells (e.g., through RNAscope®).

Detectable labels suitable for use in the methods disclosed herein include any composition detectable by spectroscopic, photochemical, biochemical, immunochemical, electrical, optical or chemical means. Useful labels include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label. The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Protein levels of MTAP can be determined by examining protein expression or the protein product. Determining the protein level involves measuring the amount of any immunospecific binding that occurs between an antibody that selectively recognizes and binds to the polypeptide of the biomarker in a sample obtained from a subject and comparing this to the amount of immunospecific binding of at least one biomarker in a control sample.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

### Adjacent Biomarkers

Near or adjacent to MTAP on chromosome 9 are several other biomarkers. CDKN2A is often, if not usually, deleted along with MTAP. Additional genes or pseudogenes in this region include: C9orf53, ERVFRD-3, TUBB8P1, KHSRPP1, MIR31, and MIR31HG.

In some embodiments of the methods, the cell that is MTAP-deficient is also deficient in CDKN2A. In some embodiments, the cell that is MTAP-deficient is also deficient in one or more of: CDKN2A, C9orf53, ERVFRD-3, TUBB8P1, KHSRPP1, MIR31, and MIR31HG.

Thus, in various methods involving a step of evaluating a cell for MTAP deficiency or determining if a cell is MTAP-deficient, this step can comprise the step of determining if the cell is deficient for one or more of these markers: CDKN2A, C9orf53, ERVFRD-3, TUBB8P1, KHSRPP1, MIR31, and MIR31HG.

Thus, in some embodiments, the disclosure encompasses: A method of determining if a subject having or having been diagnosed with a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from said subject for MTAP deficiency, and evaluating a reference sample from a non-cancerous or normal control subject for MTAP deficiency, wherein MTAP deficiency in the test sample relative to the reference sample indicates that the subject will respond to therapeutic treatment with a PRMT5 inhibitor (e.g., an MTA-uncompetitive PRMT5 inhibitor, e.g., a compound of Formula (I) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) to the subject; and d) determining the level of PRMT5 activity in the subject following step c), wherein a decrease in the level of PRMT5 activity is correlated with the inhibition of the proliferation of the cancer, and wherein steps c) and d) are performed after steps a) and b).

In some embodiments, the disclosure encompasses: A method of determining if a cancer will respond to therapeutic treatment with a PRMT5 inhibitor (e.g., an MTA-uncompetitive, non-competitive, or mixed mode PRMT5 inhibitor or an MTA-cooperative binding agent), comprising the steps of:
a) evaluating a test sample obtained from a subject having or having been diagnosed with said cancer for MTAP deficiency, and evaluating a reference sample from a non-cancerous or normal control subject for MTAP deficiency, wherein MTAP deficiency in the test sample relative to the reference sample indicates that the cancer will respond to therapeutic treatment with a PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof); wherein MTAP deficiency is evaluated by evaluating the deficiency of one or more of the following biomarkers: CDKN2A, C9orf53, ERVFRD-3, TUBB8P1, KHSRPP1, MIR31, and MIR31HG, and wherein the method can further comprise the following steps:
b) determining the level of MTAP in the subject, wherein steps a) and b) can be performed in any order;
c) administering a therapeutically effective amount of a PRMT5 inhibitor (e.g., an MTA-uncompetitive PRMT5 inhibitor, e.g., a compound of Formula (I) or a compound of Table 1, or pharmaceutically acceptable salts thereof) to the subject; and
d) determining the level of PRMT5 activity in the subject following step c), wherein a decrease in the level of PRMT5 activity is correlated with the inhibition of the proliferation of the cancer, and wherein steps c) and d) are performed after steps a) and b).

### Assaying for Biomarkers and PRMT5 Inhibitor Treatment

A number of patient stratification strategies could be employed to find patients likely to be sensitive to PRMT5 inhibition with an MTA-uncompetitive, non-competitive, or mixed mode PRMT5 inhibitor or an MTA-cooperative binding agent (e.g., a PRMT5 inhibitor of the present disclosure, e.g., a compound of Formula (I) or a compound of Table 1, or pharmaceutically acceptable salts thereof), including but not limited to, testing for MTAP deficiency and/or MTA accumulation.

Once a patient has been assayed for MTAP deficiency and/or MTA accumulation and predicted to be sensitive to treatment with a PRMT5 inhibitor, administration of any PRMT5 inhibitor (e.g., an MTA-uncompetitive, non-competitive, or mixed mode PRMT5 inhibitor or an MTA-cooperative binding agent, e.g., a compound of Formula (I) or a compound of Table 1, or pharmaceutically acceptable salts thereof) to a patient can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

### Kits

In some embodiments provided are kits related to methods of use described herein.

In one embodiment, provided is a kit for predicting the sensitivity of a subject having or having been diagnosed with an MTAP-deficiency-related cancer for treatment with a PRMT5 inhibitor is provided. The kit comprises: i) reagents capable of detecting human MTAP-deficient and/or MTA-accumulating cancer cells; and ii) instructions for how to use said kit.

### Examples

In order that the invention(s) described herein may be more fully understood, the following examples are set forth. The synthetic and biological examples described in this application are offered to illustrate the compounds, pharmaceutical compositions, and methods provided herein and are not to be construed in any way as limiting their scope. In the synthetic examples below, the descriptions of experimental procedures within a reaction sequence are listed in numerical order.

### ABBREVIATIONS

General
ADDP 1,1′-(azodicarbonyl)dipiperidine
anhy. anhydrous
aq.aqueous
satd. saturated
min(s) minute(s)
hr(s) hour(s)
mL milliliter
mmolmillimole(s)
mol mole(s)
MS mass spectrometry
NMRnuclear magnetic resonance
TLC thin layer chromatography
HPLC high-performance liquid chromatography
Me methyl
i—Pr iso-propyl
t-Bu tert-butyl
^{t}BuXPhos 2-di-tert-butylphosphino-2′,4′,6′-triisopropylbiphenyl
Ph phenyl
Et ethyl
Bz benzoyl
RuPhos 2-dicyclohexylphosphino-2′,6′-diisopropoxybiphenyl
Spectrum
Hzhertz
δ chemical shift
J coupling constant
s singlet
d doublet
t triplet
q quartet
m multiplet
br broad
qd quartet of doublets
dquin doublet of quintets
dd doublet of doublets
dt doublet of triplets

### Solvents and Reagents

DAST Diethylaminosulfurtrifluoride
CHCl₃ chloroform
DCM dichloromethane
DMF dimethylformamide
Et₂O diethyl ether
EtOH ethyl alcohol
EtOAc ethyl acetate
MeOH methyl alcohol
MeCN acetonitrile
PE petroleum ether
THF tetrahydrofuran
DMSO dimethyl sulfoxide
t-BuOK potassium tert-butoxide
9-BBN 9-borabicyclo[3.3.1]nonane
AcOH acetic acid
HCl hydrochloric acid
H₂SO₄ sulfuric acid
NH₄Cl ammonium chloride
KOH potassium hydroxide
NaOH sodium hydroxide
K₂CO₃ potassium carbonate
Na₂CO₃ sodium carbonate
TFA trifluoroacetic acid
Na₂SO₄ sodium sulfate
NaBH4 sodium borohydride
NaHCO₃sodium bicarbonate
LiHMDS lithium hexamethyldisilylamide
NaBH4 sodium borohydride
Et3N triethylamine
Py pyridine
PCC pyridinium chlorochromate
DMAP 4-(dimethylamino)pyridine
DIPEA N,N-diisopropylethylamine
BINAP 2,2′-bis(diphenylphosphanyl)-1,1′-binaphthyl
dppf 1,1′-bis(diphenylphosphino)ferrocene
PEP Phospho(enol)pyruvic acid
LDH Lactate Dehydrogenase
DTT DL-Dithiothreitol
BSA Bovine Serum Albumin
NADH P-Nicotinamide adenine dinucleotide, reduced
Pd(t-Bu₃P)₂ bis(tri-tert-butylphosphine)palladium(O)
AcCl acetyl chloride
i—PrMgCl Isopropylmagnesium chloride
TBSCl tert-Butyl(chloro)dimethylsilane
(i—PrO)₄Ti titanium tetraisopropoxide
BHT 2,6-di-t-butyl-4-methylphenoxide
BzCl benzoyl chloride
CsF cesium fluoride
DCC dicyclohexylcarbodiimide
DMP Dess-Martin periodinane
EtMgBr ethylmagnesium bromide
EtOAc ethyl acetate
TEA triethylamine
AlaOH alanine
TBAF tetra-n-butylammonium fluoride
TBS t-butyldimethylsilyl
TMS trimethylsilyl
TMSCF₃ (Trifluoromethyl)trimethylsilane
Ts p-toluenesulfonyl
Bu butyl
Ti(OⁱPr)₄ tetraisopropoxytitanium
LAH Lithium Aluminium Hydride
LDA lithium diisopropylamide
LiOH·H₂O lithium hydroxide hydrates
MAD methyl aluminum bis(2,6-di-t-butyl-4-methylphenoxide)
NBS N-bromosuccinimide
Na₂SO₄ sodium sulfate
Na₂S₂O₃ sodium thiosulfate
PE petroleum ether
MeCN acetonitrile
Boc t-butoxycarbonyl
MTBE methyl tert-butyl ether
DIAD diisopropyl azodicarboxylate

### General Experimental Notes:

In the following examples, the chemical reagents were purchased from commercial sources (such as Alfa, Acros, Sigma Aldrich, TCI and Shanghai Chemical Reagent Company), and used without further purification.

In some examples, purification of intermediates and final compounds was performed using HPLC (H₂O - MeOH; Agilent 1260 Infinity systems equipped with DAD and mass-detectors. Waters Sunfire C18 0BD Prep Column, 100A, 5 μm, 19 mm×100 mm with SunFire C18 Prep Guard Cartridge, 100A, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated under the N₂ flow upon heating to 80° C. On the basis of post-chromatography LCMS analysis fractions were united. Solid fractions were dissolved in 0.5 mL MeOH and transferred into pre-weighted marked vials. Obtained solutions were again evaporated under the N₂ flow upon heating to 80° C. After drying, products were subjected to lyophilization using acetonitrile-water mixtures and finally characterized by LCMS and ¹H NMR.

Nuclear magnetic resonance (NMR) spectra were recorded using Brucker AVANCE DRX 500, Bruker 400 spectrometer or Varian UNITYplus 400. Chemical shifts for protons were reported as parts per million in 8 scale using solvent residual peak (CHCl₃: 7.27 ppm) (methanol-d4: 3.31 ppm) (DMSO-d6: 2.50 ppm) or tetramethylsilane (0.00 ppm) as internal standards. Chemical shifts of ¹³C NMR spectra were reported in ppm from the central peak of CDCl3 (77.00 ppm) (methanol-d4: 49.15 ppm) (DMSO-d₆: 39.51 ppm) on the 8 scale. Data are represented as follows: chemical shift, multiplicity (s=singlet, d=doublet, t=triplet, q=quartet, qn=quintuplet, sx=sextet, sp=septuplet, m=multiplet, br=broad), coupling constant (J, Hz) and integration.

In certain examples, mass spectra were recorded on an Agilent 1100 Series LC/MSD system with DADELSD and Agilent LCMSD VL (G1956A), SL (G1956B) mass-spectrometer or an Agilent 1200 Series LC/MSD system with DADELSD and Agilent LCMSD SL (G6130A), SL (G6140A) mass-spectrometer.

All the LC/MS data were obtained using positive/negative mode switching.
Column Zorbax SB-C18 1.8 μm 4.6×15 mm Rapid Resolution cartridge (PN 821975-932) Mobile phase A—acetonitrile, 0.1% formic acid
B—water (0.1% formic acid)
Flow rate 3 ml/min
Gradient 0 min—100% B
0.01 min—100% B
1.5 min—0% B
1.8 min—0% B
1.81 min—100% B
Injection volume 1 μl
Ionization mode atmospheric pressure chemical ionization (APCI)
Scan range m/z 80-1000.

Other exemplary analytical LC/MS instruments and conditions are described below:

Instrument: Agilent LC1100-MS6100 series G1956B; Column: Xbridge Shield RP-18, 50*2.1 mm*5 μm; Mobile Phase A: H₂O with 0.05% NH₃—H₂O (v %); Mobile Phase B: MeCN; Flow rate: 1.0 mL/min; Wavelength: UV 220 nm, 254 nm; Column temperature: 30° C.; MS ionization: ESI.
0˜30CD: Gradient: B from 0%˜30% over 2 minutes and holding at 30% for 0.48 minutes;
0-60CD: Gradient: B from 0%˜60% over 2 minutes and holding at 60% for 0.48 minutes;
10-80CD: Gradient: B from 10%˜80% over 2 minutes and holding at 80% for 0.48 minutes;
30-90CD: Gradient: B from 30%˜90% over 2 minutes and holding at 90% for 0.48 minutes;
50-100CD: Gradient: B from 50%˜100% over 2 minutes and holding at 100% for 0.48 minutes.

Instrument: Agilent LC1100-MS6100 series G1956B; Column: Xtimate C18, 30*2.1 mm*3 m; Mobile Phase A: H₂O with 0.0375% TFA (v %); Mobile Phase B: MeCN with 0.01875% TFA (v %): Flow rate: 0.8 mL/min; Wavelength: UV 220 nm, 254 nm; Column temperature: 50° C.; MS ionization: ESI.
0˜30AB: Gradient: B from 0%˜30% over 3 minutes and holding at 30% for 0.5 minutes;
0-60AB: Gradient: B from 0%˜60% over 3 minutes and holding at 30% for 0.5 minutes;
10-80AB: Gradient: B from 10%˜80% over 3 minutes and holding at 30% for 0.5 minutes;
30-90AB: Gradient: B from 0%˜30% over 3 minutes and holding at 30% for 0.5 minutes;
50-100AB: Gradient: B from 50% 100% over 3 minutes and holding at 100% for 0.5 minutes.

Instrument: Shimadzu LC20-MS2010; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm, 215 nm; Column temperature: 50° C.; MS ionization: ESI.

Instrument: Shimadzu LC20-MS2020; Column: Agilent Pursit 5 C18 20*2.0 mm; Mobile Phase A: H₂O with 0.0375% of TFA (v %); Mobile Phase B: MeCN with 0.01875% of TFA (v %); Gradient: B from 5˜95% over 0.7 minutes and holding at 95% for 0.4 minutes; Flow Rate: 1.5 mL/min; Wavelength: UV 220 nm, 254 nm; Column temperature: 50° C.; MS ionization: ESI.

### Exemplary HPLC Instruments and Conditions

Instrument: Shimadzu LC20; Column: YMC-Pack ODS-A 150*4.6 mm; Mobile Phase A: H₂O with 0.06875% TFA (v %); Mobile Phase B: MeCN with 0.0625% TFA (v %); Flow rate: 1.5 mL/min; Wavelength: UV 220 nm, 215 nm, 254 nm; Column temperature: 40° C.
0˜30: Gradient: B from 0˜30% over 10 minutes and holding at 30% for 5 minutes;
0˜60: Gradient: B from 0˜60% over 10 minutes and holding at 60% for 5 minutes;
0-95: Gradient: B from 0˜95% over 10 minutes and holding at 95% for 5 minutes;
10-80: Gradient: B from 10˜80% over 10 minutes and holding at 80% for 5 minutes;
30-90: Gradient: B from 30˜90% over 10 minutes and holding at 90% for 5 minutes;
50-100: Gradient: B from 50˜100% over 10 minutes and holding at 100% for 5 minutes.

Instrument: Shimadzu LC20; Column: Xbridge Shield RP-18 50*2.1 mm, 5 μm; Mobile Phase A: H₂O with 0.01% NH₃—H₂O; Mobile Phase B: MeCN; Flow Rate: 1.2 mL/min; Wavelength: UV 220 nm, 215 nm, 254 nm; Column temperature: 40° C.
0˜30CD: Gradient: B from 0˜30% over 6 minutes and holding at 30% for 2 minutes;
0-60CD: Gradient: B from 0˜60% over 6 minutes and holding at 60% for 2 minutes;
10-80CD: Gradient: B from 10˜80% over 6 minutes and holding at 80% for 2 minutes;
30-90CD: Gradient: B from 30˜90% over 6 minutes and holding at 90% for 2 minutes;
50-100CD: Gradient: B from 10˜80% over 6 minutes and holding at 100% for 2 minutes.

Instrument: Shimadzu LC20; Column: Ultimate C18 50 * 3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
0˜30AB: Gradient: B from 0˜30% over 2.5 minutes and holding at 30% for 0.75 minutes;
0-60AB: Gradient: B from 0˜60% over 2.5 minutes and holding at 60% for 0.75 minutes; 5-95AB: Gradient: B from 5˜95% over 2.5 minutes and holding at 95% for 0.75 minutes.

Instrument: Shimadzu LC20; Column: Ultimate C18 50 * 3 mm, 3 μm; Mobile Phase A: H₂O with 0.06875% TFA (v %); Mobile Phase B: MeCN with 0.0625% TFA (v %); Flow Rate: 1.2 mL/min; Wavelength: UV 220 nm, 215 nm, 254 nm; Column temperature: 40° C.
10-80AB: Gradient: B from 10˜80% over 4 minutes and holding at 80% for 2 minutes.

### Exemplary TLC, Concentration and Normal Phase Chromatography.

Analytical thin layer chromatography (TLC) was performed with silica gel 60 F254 aluminum plates. Visualization was done under a UV lamp (254 nm) and by iodine or immersion in ethanolic phosphomolybdic acid (PMA) or potassium permanganate (KMnO4), followed by heating using a heat gun. Organic solutions were concentrated by rotary evaporation at 20-40° C. Purification of reaction products were generally done by flash column chromatography with 230-400 mesh silica gel or Agela flash silica column.

### Exemplary Chiral SFC Analytical Methods

Column: Chiralpak AD-3 150×4.6 mm I.D., 3 m; Mobile phase: A: supercritical CO₂; Mobile phase B: EtOH (0.05% DEA); Gradient: from 5% to 40% of B in 5 min and hold 40% for 2.5 min, then 5% of B for 2.5 min; Flow rate: 2.5 mL/min; Column temperature: 35° C.; ABPR: 1500 psi.

Column: Chiralpak AD-3 100×4.6 mm I.D., 3 m; Mobile phase: A: supercritical C₀₂ Mobile phase B: EtOH (0.1% ethanolamine); Gradient: from 5% to 40% of B in 4.5 min and hold 40% for 2.5 min, then 5% of B for 1 min; Flow rate: 2.8 mL/min; Column temperature: 40° C.

### Exemplary Preparative HPLC Separation Methods

Basic condition (NH₃—H₂O): Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %); Mobile phase B: MeCN; Gradient: B from 22% to 52% in 9.5 min, hold 100% B for 1 min; Flow Rate: 25 mL/min; Column Temperature: 30° C.; Wavelength: 220 nm, 254 nm.

Acid condition (HCOOH): Instrument: Gilson GX-281 Liquid Handler, Gilson 322 Pump, Gilson 156 UV Detector; Column: Agela Durashell C18 150*25 mm 5 μm; Mobile phase A: H₂O (0.0225% HCOOH); Mobile phase B: MeCN; Gradient: B from 7% to 37% in 9 min, hold 100% B for 0 min; Flow Rate: 25 mL/min; Column Temperature: 30° C.; Wavelength: 220 nm, 254 nm.

Acid condition (HCl): Gilson GX-281 Liquid Handler, Gilson 322 Pump, Gilson 156 UV Detector; Column: Xtimate C18 150*25 mm*5 m; Mobile phase A: H₂O with 0.05% HCl (v %); Mobile phase B: MeCN; Gradient: B from 0% to 30% in 6.5 min, hold 100% B for 2.5 min; Flow Rate: 25 mL/min; Column Temperature: 30° C.; Wavelength: 220 nm, 254 nm).

Neutral condition (NH₄HCO3): (Instrument: Gilson GX-281 Liquid Handler, Gilson 322 Pump, Gilson 156 UV Detector; Column: Waters Xbridge 150×25 mm×5 μm; Mobile phase A: H₂O with 10 mmol NH₄HCO3; Mobile phase B: MeCN; Gradient: B from 39% to 69% in 10 min, hold 100% B for 2.5 min; Flow Rate: 25 mL/min; Column Temperature: 30° C.; Wavelength: 220 nm, 254 nm).

### Exemplary Large-Scale Separation

Basic condition: Instrument: Shimadzu LC-8A Pumps, Shimadzu SCL-10A VP System Controller, Shimadzu SPD-20AV UV/VIS Detector; Column: Phenomenex Gemini C18 250*50 mm*10 m; Mobile phase A: water (0.04% NH₃-H₂O+10 mM NH₄HCO3); Mobile phase B: MeCN; Gradient: B from 65% to 95% in 26 min, hold 100% B for 3 min; Flow Rate: 110 mL/min; Column Temperature: 30° C.; Wavelength: 220 nm, 254 nm.

Acid condition (TFA): Instrument: Shimadzu LC-20AP Pumps, Shimadzu CBM-20A System Controller Shimadzu SPD-20AV UV/VIS Detector; Column: Phenomenex luna C18 250×50 mm×10 m; Mobile phase A: H₂O with 0.1% TFA (v %); Mobile phase B: MeCN; Gradient: B from 0% to 25% in 15 min, hold 100% B for 4 min; Flow Rate: 120 mL/min; Column Temperature: 30° C.; Wavelength: 220 nm, 254 nm.

### Exemplary Preparative Chiral SFC Method:

Exemplary chiral columns available for use in the separation/purification of the enantiomers/diastereomers provided herein include, but are not limited to, CHIRALPAK® AD-10, CHIRALCEL® OB, CHIRALCEL® OB—H, CHIRALCEL® OD, CHIRALCEL® OD-H, CHIRALCEL® OF, CHIRALCEL® OG, CHIRALCEL® OJ and CHIRALCEL® OK.

In certain examples, the chiral separation was performed under the following conditions: Instrument: Thar 80; Column: Daicel Chiralpak AD. 250×30 mm I.D. 10 μm; Mobile phase: supercritical C₀₂/MeOH (0.1 1% NH₃—H₂O, v %)=60/40; Flow Rate: 70 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative pyrazoles that have been listed herein. The compounds provided herein may be prepared from known or commercially available starting materials and reagents by one skilled in the art of organic synthesis.

Exemplary general method for preparative HPLC: Column: Waters RBridge prep 10 m C18, 19*250 mm. Mobile phase: acetonitrile, water (NH₄HCO3) (30 L water, 24 g NH₄HCO3, 30 mL NH₃.H₂O). Flow rate: 25 mL/min

Exemplary general method for analytical HPLC: Mobile phase: A: water (10 mM NH₄HCO3), B: acetonitrile Gradient: 5%-95% B in 1.6 or 2 min Flow rate: 1.8 or 2 mL/min; Column: XBridge C18, 4.6*50 mm, 3.5 m at 45° C.

### Intermediates

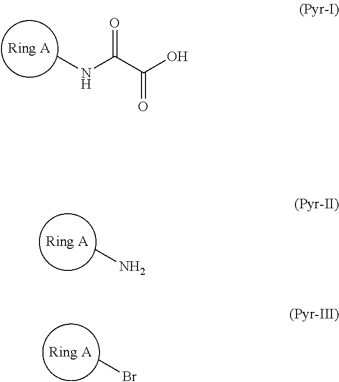

Intermediates Pyr-I, Pyr-II, and Pyr-III were synthesized according to conventional procedures from patent and non-patent literature.

### Intermediate 1. 2-oxo-2-[[1-(2-trimethylsilylethoxvmethyl)pyrazolo[4,3-c]pyridin-7-yl]aminolacetic acid

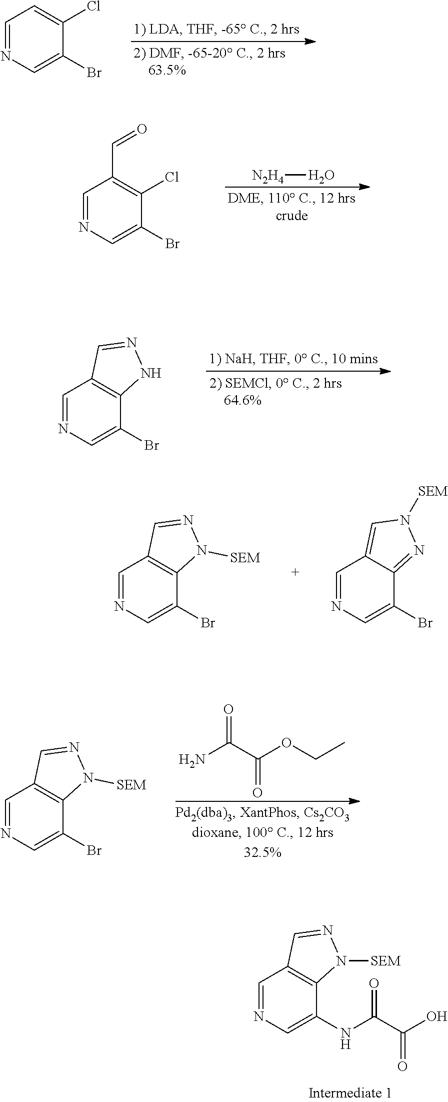

### Step 1: Synthesis of 5-bromo-4-chloro-pyridine-3-carbaldehyde

To a mixture of 2M LDA/THF (35 mL, 70.0 mmol) in THF (20 mL) was added 3-bromo-4-chloro-pyridine (11 g, 57.2 mmol) dropwise at −65° C. under N₂. The resulting mixture was stirred at −65° C. for 2 hours. N,N-dimethylformamide (5.4 mL, 69.7 mmol) was added at −65° C. and the reaction was stirred at 20° C. for 2 hours. The resulting mixture was quenched by addition of NH₄Cl aqueous solution (100 mL) and extracted with EtOAc (100 mL*3). The combined organic layer was washed with saturated NH₄Cl aqueous solution (100 mL), brine (100 mL), dried over anhydrous Na₂SO₄, filtered and concentrated under reduced pressure. The residue was purified by flash chromatography (ISCO®; 80 g AgelaFlash® Silica Flash Column, petroleum ether/EtOAc with EtOAc from 0˜30%, flow rate=30 mL/min, 254 nm) to afford 5-bromo-4-chloro-pyridine-3-carbaldehyde (8 g, 63.5% yield) as yellow solid. ¹H NMR (400 MHz, DMSO-d₆) δ ppm 10.22-10.40 (m, 1H), 9.02-9.18 (m, 1H), 8.83-8.95 (m, 1H); LCMS (ESI) [M+H]⁺ m/z: calcd 221.9, found 221.9.

### Step 2: Synthesis of 7-bromo-1H-pyrazolo[4,3-c]pyridine

A mixture of 5-bromo-4-chloro-pyridine-3-carbaldehyde (6.1 g, 27.7 mmol) in hydrazine (10 mL, 17.0 mol) and DME (40 mL) was stirred at 110° C. for 12 hours. The resulting mixture was filtered and concentrated under reduced pressure to give 7-bromo-1H-pyrazolo[4,3-c]pyridine (5.6 g, crude) as yellow solid. ¹H NMR (400 MHz, methanol-d4) δ ppm 9.05 (s, 1H), 8.42 (d, J=13.1 Hz, 2H).

### Step 3: Synthesis of 2-[(7-bromopyrazolo[4,3-c]pyridin-1-yl)methoxy]ethyl-trimethyl-silane and 2-[(7-bromopyrazolo[4,3-c]pyridin-2-yl)methoxy]ethyl-trimethyl-silane

To a mixture of 7-bromo-1H-pyrazolo[4,3-c]pyridine (5.6 g, 28.3 mmol) in DMF (60 mL) was added NaH (2 g, 50.0 mmol, 60 wt % in mineral oil) slowly at 0° C., the mixture was stirred at 0° C. for 10 minutes. Then 2-(chloromethoxy)ethyl-trimethyl-silane (5.6 mL, 31.6 mmol) was added dropwise. The resulting mixture was stirred at 0° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 40 g AgelaFlash® Silica Flash Column, petroleum ether/EtOAc with EtOAc from 0˜15%, flow rate=50 mL/min, 254 nm) to afford P1 and P2.

P1: 2-[(7-bromopyrazolo[4,3-c]pyridin-1-yl)methoxy]ethyl-trimethyl-silane (6 g, 64.6% yield) was obtained as yellow oil. ¹H NMR (400 MHz, methanol-d4) δ ppm 9.05 (s, 1H), 8.53 (s, 1H), 8.37 (s, 1H), 6.06 (s, 2H), 3.64 (t, J=7.9 Hz, 2H), 0.85 (t, J=7.9 Hz, 2H), −0.09 (s, 9H); LCMS (ESI) [M+H]⁺ m/z: calcd 330.0, found 329.9.
P2: 2-[(7-bromopyrazolo[4,3-c]pyridin-2-yl)methoxy]ethyl-trimethyl-silane (1 g, 10.8% yield) as yellow oil. LCMS (ESI) [M+H]⁺ m/z: calcd 330.0, found 329.9.

### Step 4: Synthesis of 2-oxo-2-[[1-(2-trimethylsilylethoxymethyl)pyrazolo[4,3-c]pyridin-7-yl]amino]acetic acid

To a mixture of 2-[(7-bromopyrazolo[4,3-c]pyridin-1-yl)methoxy]ethyl-trimethyl-silane (6 g, 18.3 mmol) and ethyl 2-amino-2-oxo-acetate (6.42 g, 54.8 mmol) in dioxane (70 mL) were added Cs₂CO₃ (9.09 g, 27.9 mmol), XantPhos (1.80 g, 3.61 mmol) and Pd₂(dba)₃ (1.68 g, 1.83 mmol). The resulting mixture was sealed and degassed under vacuum and purged with N₂ for three times, and then stirred at 100° C. for 12 hours under N₂ atmosphere. The resulting mixture was quenched by addition of water (50 mL) and extracted with EtOAc (100 mL*2). The combined water layer was concentrated under reduced pressure. The residue was purified by flash chromatography (Biotage®, Column: SepaFlash® Spherical C18, 60 g, 40-60 m, 120 Å; MeCN/water (0.05 v % NH₃—H₂O) with MeCN from 0-23%, 25 10 mL/min, 254 nm) to give 2-oxo-2-[[1-(2-trimethylsilylethoxymethyl)pyrazolo[4,3-c]pyridin-7-yl]amino]acetic acid (2 g, 32.5% yield) as yellow solid. ¹H NMR (400 MHz, methanol-d4) δ ppm 8.93 (d, J=13.1 Hz, 2H), 8.31 (s, 1H), 5.92 (s, 2H), 3.56-3.62 (m, 2H), 0.89-0.99 (m, 2H), −0.08 (s, 9H); LCMS (ESI) [M+H]⁺ m/z: calcd 337.1, found 337.0.

### Intermediate 2. 5-Iodo-3-(oxetan-3-yl)pyridin-2-amine

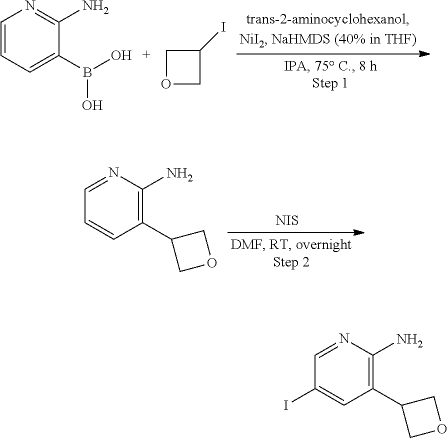 [SMILES: Nc1ncccc1B(O)O ; IC1COC1 ; Nc1ncccc1C1COC1 ; "NIS" ; Nc1ncc(I)cc1C1COC1]

### Step 1: The synthesis of 3-(oxetan-3-yl)pyridin-2-amine

### Procedure:

trans-2-Aminocyclohexanol (198.13 mg, 1.72 mmol) and Nickel (II) iodide (537.58 mg, 1.72 mmol) were added to a solution of (2-amino-3-pyridyl)boronic acid (5 g, 28.67 mmol, HCl) in isopropyl alcohol (50 mL). Then, Sodium bis(trimethylsilyl)amide (40% in THF) (39.43 g, 86.01 mmol, 44.11 mL, 40% purity) was added dropwise followed by 3-iodooxetane (7.91 g, 43.01 mmol, 3.70 mL). The reaction flask was purged with argon and the resulting mixture was stirred at 75° C. for 8 hr.

### Work-Up:

The volatiles were removed under reduced pressure and residue was purified by gradient column chromatography

### Purification:

Companion combiflash; 80 g SiO₂, CHCl₃-MeOH from 0˜100%, flow rate=60 mL/min, cv=

### Conclusion:

3-(Oxetan-3-yl)pyridin-2-amine (1 g, 6.66 mmol, 23.22% yield) was obtained as yellow oil.

¹H NMR (400 MHz, DMSO-d6) δ 4.12 (q, 1H), 4.62 (dd, 2H), 4.90 (dd, 2H), 5.69 (s, 2H), 6.62 (dd, 1H), 7.45 (s, 1H), 7.83 (s, 1H).

LCMS(ESI): [M+H]⁺ m/z: calcd 150.2; found 151.0; Rt=0.166 min.

### Step 2: The synthesis of 5-iodo-3-(oxetan-3-yl)pyridin-2-amine

### Procedure:

3-(Oxetan-3-yl)pyridin-2-amine (0.8 g, 5.33 mmol) was dissolved in DMFA (15 mL) followed by portion-wise addition of 1-iodopyrrolidine-2,5-dione (1.60 g, 7.11 mmol). The reaction mixture was stirred overnight and concentrated under reduced pressure.

### Work-Up:

The residue was dissolved in DCM and the organic layer was washed with Na₂S2O₃ aqueous solution and water multiple times. DCM layer was separated, dried over Na₂SO₄ and concentrated under reduced pressure.

### Conclusion:

Pure 5-iodo-3-(oxetan-3-yl)pyridin-2-amine (0.6 g, 2.17 mmol, 40.80% yield) was obtained as yellow solid.

¹H NMR (400 MHz, DMSO-d6) δ 4.15 (q, 1H), 4.56 (dd, 2H), 4.87 (dd, 2H), 5.87 (s, 1H), 7.66 (s, 1H), 7.98 (s, 1H).

LCMS(ESI): [M+H]⁺ m/z: calcd 277.0; found 277.0; Rt=0.563 min.

### Example 1. Compound 111 N-(6-amino-5-methyl-3-pyridyl)-2-[(2R,5R)-5-methyl-2-(2-thienyl)-4-[1-(trifluoromethyl)cyclopropanecarbonyl]piperazin-1-yl]-2-oxo-acetamide

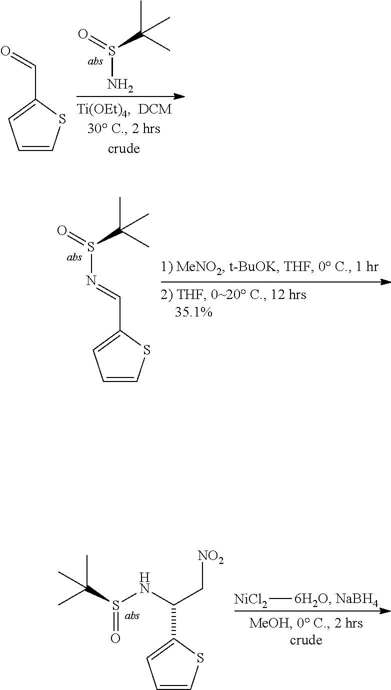

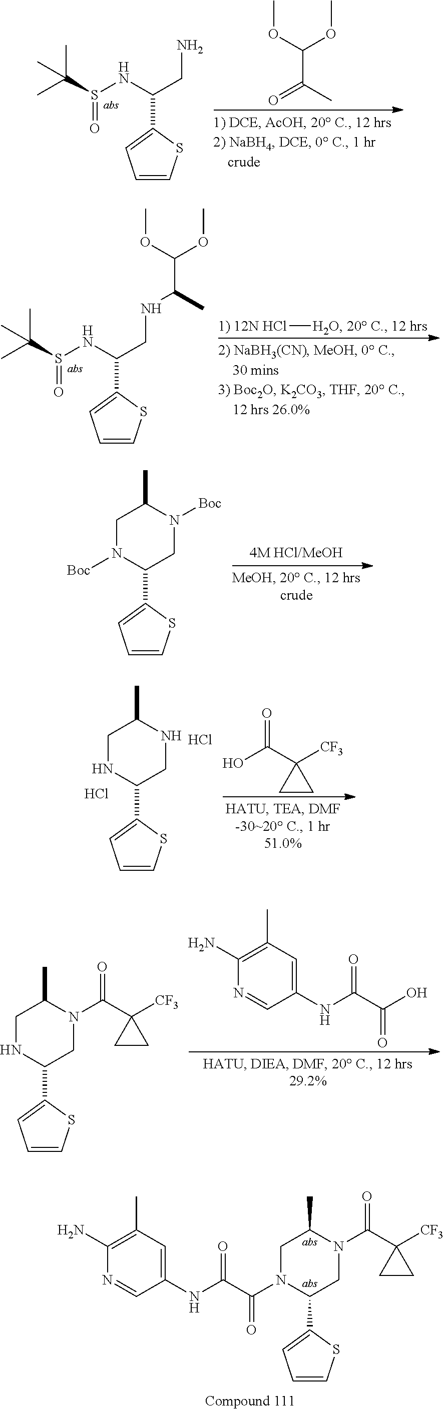

### Step 1: Synthesis of (NE)-2-methyl-N-(2-thienylmethylene)propane-2-sulfinamide

A mixture of thiophene-2-carbaldehyde (5 g, 44.6 mmol), 2-methylpropane-2-sulfinamide (6.48 g, 53.5 mmol) and Ti(OEt)₄ (18.0 mL, 86.0 mmol) in DCM (50 mL) was stirred at 30° C. for 2 hours. The resulting mixture was quenched by addition of water (200 mL) and extracted with DCM (200 mL*3). The combined organic layer was washed with brine (100 mL), dried over anhydrous Na₂SO₄, filtered and concentrated under reduced pressure to give (NE)-2-methyl-N-(2-thienylmethylene)propane-2-sulfinamide (8 g, crude) as yellow solid. ¹H NMR (400 MHz, methanol-d₄) δ ppm 8.66 (s, 1H), 7.80 (d, J=5.0 Hz, 1H), 7.67-7.75 (m, 1H), 7.22 (dd, J=4.8, 3.8 Hz, 1H), 1.24 (s, 9H); HPLC: 91.03% at 254 nm; 100% ee.

### Step 2: Synthesis of 2-methyl-N-[(1R)-2-nitro-1-(2-thienyl)ethyl]propane-2-sulfinamide

A mixture of 1M tBuOK/THF (37 mL, 37 mmol) in THF (20 mL) was cooled to 0° C. then the nitromethane (8 mL, 0.148 mol) was added to the mixture, then the solution was stirred at 0° C. for 1 hour. Then (NE)-2-methyl-N-(2-thienylmethylene)propane-2-sulfinamide (2 g, 9.29 mmol) was added at 0° C. and the mixture was stirred at 20° C. for 12 hours. The residue was purified by flash chromatography (ISCO®; 20 g AgelaFlash® Silica Flash Column, petroleum ether/EtOAc with EtOAc from 0˜50%, flow rate=30 mL/min, 254 nm) to afford 2-methyl-N-[(1R)-2-nitro-1-(2-thienyl)ethyl]propane-2-sulfinamide (0.9 g, 35.1% yield) as brown oil.

LCMS (ESI) [M+H]⁺ m/z: calcd 277.1, found 277.0.

### Step 3: Synthesis of N-[(1R)-2-amino-1-(2-thienyl)ethyl]-2-methyl-propane-2-sulfinamide

To a mixture of 2-methyl-N-[(1R)-2-nitro-1-(2-thienyl)ethyl]propane-2-sulfinamide (900 mg, 3.26 mmol), NiCl-6H₂O (850 mg, 3.58 mmol) in MeOH (10 mL) was added NaBH4 (620 mg, 16.4 mmol). The resulting mixture was stirred at 0° C. for 2 hours. The resulting mixture was filtered and concentrated under reduced pressure to give N-[(1R)-2-amino-1-(2-thienyl)ethyl]-2-methyl-propane-2-sulfinamide (600 mg, crude) as brown oil. LCMS (ESI) [M+H]⁺ m/z: calcd 247.1, found 247.1.

### Step 4: Synthesis of N-[2-[(2,2-dimethoxy-1-methyl-ethyl)amino]-1-(2-thienyl)ethyl]-2-methyl-propane-2-sulfinamide

A mixture of N-[(1R)-2-amino-1-(2-thienyl)ethyl]-2-methyl-propane-2-sulfinamide (600 mg, 2.44 mmol), DCE (10 mL) and AcOH (0.14 mL, 2.45 mmol) was stirred at 20° C. for 12 hours. NaBH4 (280 mg, 7.40 mmol) was added at 0° C. The resulting mixture was stirred at 0° C. for 1 hour. The resulting mixture was quenched by addition of water (30 mL) and extracted with DCM (100 mL*3). The combined organic layer was washed with brine (50 mL), dried over anhydrous Na₂SO₄, filtered and concentrated under reduced pressure to give N-[2-[(2,2-dimethoxy-1-methyl-ethyl)amino]-1-(2-thienyl)ethyl]-2-methyl-propane-2-sulfinamide (700 mg, crude) as brown solid. LCMS (ESI) [M+H]⁺ m/z: calcd 349.2, found 349.1.

### Step 5: Synthesis of ditert-butyl 2-methyl-5-(2-thienyl)piperazine-1,4-dicarboxylate

A mixture of N-[2-[(2,2-dimethoxy-1-methyl-ethyl)amino]-1-(2-thienyl)ethyl]-2-methyl-propane-2-sulfinamide (700 mg, 2.01 mmol) in 12N HCl/H₂O (5 mL, 60 mmol) was stirred at 20° C. for 12 hours. The resulting mixture was concentrated under reduced pressure to give a residue, which was mixed with MeOH (5 mL) and Na₂SO₄ (850 mg, 5.98 mmol), then NaBH3(CN) (130 mg, 2.07 mmol) was added at 0° C. slowly, and the mixture was stirred at 0° C. for 30 minutes. The resulting mixture was concentrated under reduced pressure and the residue was dissolved in THF (10 mL), followed by addition of (Boc)₂O (1.5 g, 6.87 mmol) and K2CO₃ (830 mg, 6.01 mmol). The solution was stirred at 20° C. for 12 hours. The resulting mixture was quenched by addition of water (50 mL) and extracted with EtOAc (100 mL*3). The combined organic layer was washed with saturated NH₄Cl aqueous solution (100 mL), brine (100 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜10%, flow rate=30 mL/min, 254 nm) to afford ditert-butyl 2-methyl-5-(2-thienyl)piperazine-1,4-dicarboxylate (200 mg, 26.0% yield) as colorless oil. LCMS (ESI) [M+H]f m/z: calcd 393.2, found 327.1 (Boc and t-Bu cleaved mass).

### Step 6: Synthesis of 2-methyl-5-(2-thienyl)piperazine (

A mixture of ditert-butyl 2-methyl-5-(2-thienyl)piperazine-1,4-dicarboxylate (195 mg, 0.510 mmol) and 4M HCl/MeOH (2 mL, 8 mmol) in MeOH (2 mL) was stirred at 20° C. for 12 hours. The resulting mixture was concentrated under reduced pressure to give 2-methyl-5-(2-thienyl)piperazine (130 mg, crude, 2HCl) as white solid. LCMS (ESI) [M+H]⁺ m/z: calcd 183.1, found 183.0.

### Step 7: Synthesis of [2-methyl-5-(2-thienyl)piperazin-1-yl]-[1-(trifluoromethyl)cyclopropyl]methanone

To a mixture of 2-methyl-5-(2-thienyl)piperazine (110 mg, 0.431 mmol, 2HCl) in DMF (3 mL) were added HATU (195 mg, 0.513 mmol) and TEA (0.3 mL, 2.15 mmol). Then 1-(trifluoromethyl)cyclopropanecarboxylic acid (55.0 mg, 0.357 mmol) was added at −30° C. for 1 hour. The resulting mixture was stirred at 20° C. for 1 hour. The mixture was concentrated under reduced pressure to give a crude product, which was purified by flash chromatography (Column: SepaFlash® Spherical C18, 40 g, 40-60 m, 120 Å; MeCN/water (0.05 v % NH₃—H₂O) with MeCN from 0˜50%, 50 mL/min, 254 nm) to afford [2-methyl-5-(2-thienyl)piperazin-1-yl]-[1-(trifluoromethyl)cyclopropyl]methanone (70 mg, 51.0% yield) as a white solid. LCMS (ESI) [M+H]⁺ m/z: calcd 319.1, found 319.2.

### Step 8: Synthesis of N-(6-amino-5-methyl-3-pyridyl)-2-[(2R,5R)-5-methyl-2-(2-thienyl)-4-[1-(trifluoromethyl)cyclopropanecarbonyl]piperazin-1-yl]-2-oxo-acetamide (Compound 111)

A mixture of [2-methyl-5-(2-thienyl)piperazin-1-yl]-[1-(trifluoromethyl)cyclopropyl]methanone (70.0 mg, 0.220 mmol), 2-[(6-amino-5-methyl-3-pyridyl)amino]-2-oxo-acetic acid (50 mg, 0.256 mmol), HATU (100 mg, 0.263 mmol) and DIPEA (0.18 mL, 1.03 mmol) in DMF (4 mL) was stirred at 20° C. for 12 hours. The mixture was concentrated under reduced pressure to give a crude product, which was purified by preparative HPLC (Instrument: Gilson GX-281 Liquid Handler, Gilson 322 Pump, Gilson 156 UV Detector; Column: Phenomenex Gemini-NX 80*40 mm*3 m; Mobile phase A: H₂O with 10 mM NH₄HCO3 (v %); Mobile phase B: MeCN; Gradient: B from 23% to 53% in 9.5 min, hold 100% B for 2 min; Flow Rate: 25 mL/min; Column Temperature: 30° C.; Wavelength: 220 nm, 254 nm) to afford product as a white solid. The product was further purified by preparative HPLC (Instrument: Gilson GX-281 Liquid Handler, Gilson 322 Pump, Gilson 156 UV Detector; Column: Welch Xtimate C18 150*25 mm*5 m; Mobile phase A: H₂O with 0.225% FA (v %); Mobile phase B: MeCN; Gradient: B from 15% to 55% in 7.8 min, hold 100% B for 1 min; Flow Rate: 25 mL/min; Column Temperature: 30° C.; Wavelength: 220 nm, 254 nm) to afford N-(6-amino-5-methyl-3-pyridyl)-2-[(2R,5R)-5-methyl-2-(2-thienyl)-4-[1-(trifluoromethyl)cyclopropanecarbonyl]piperazin-1-yl]-2-oxo-acetamide (31.8 mg, single known enantiomer with trans relative chemistry, white solid, 29.2% yield). ¹H NMR (400 MHz, methanol-d₄) δ ppm 8.15 (br s, 1H), 7.58-7.79 (m, 1H), 7.37 (br d, J=4.1 Hz, 1H), 6.91-7.13 (m, 2H), 6.05 (br s, 1H), 4.92 (br s, 1H), 4.66-4.75 (m, 1H), 4.05-4.33 (m, 1H), 3.42-3.61 (m, 1H), 2.95-3.27 (m, 1H), 2.17 (s, 3H), 1.10-1.49 (m, 7H); ¹⁹F NMR (376 MHz, methanol-d₄)₆ ppm -68.67; LCMS (ESI) [M+H]⁺ m/z: calcd 496.2, found 496.2; HPLC: 100% at 220 nm, 100% at 254 nm; 100% ee.

### Example 2. Compound 141 N-(6-amino-5-cyclopropyl-3-pyridyl)-2-[(2S,5R)-2-(1,3-benzothiazol-5-yl)-5-methyl-4-[1-(trifluoromethyl)cyclopropanecarbonyl]piperazin-1-yl]-2-oxo-acetamide

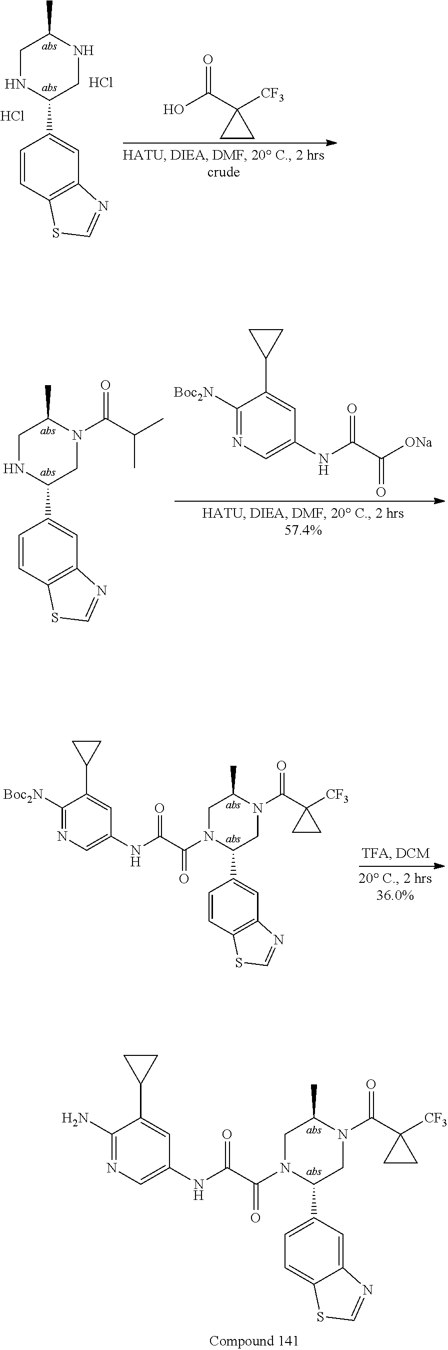

### Step 1: Synthesis of [(2R,5S)-5-(1,3-benzothiazol-5-yl)-2-methyl-piperazin-1-yl]-[1-(trifluoromethyl)cyclopropyl]methanone

To a mixture of 5-[(2S,5R)-5-methylpiperazin-2-yl]-1,3-benzothiazole (50.0 mg, 0.214 mmol), 1-(trifluoromethyl)cyclopropanecarboxylic acid (34 mg, 0.221 mmol), HATU (110 mg, 0.289 mmol) and DMF (3 mL) was added DIPEA (0.2 mL, 1.15 mmol) and the mixture was stirred at 20° C. for 2 hours. The mixture was purified by flash chromatography (Biotage®, Column: SepaFlash®Spherical C18, 25 g, 40-60 m, 120 Å; MeCN/water (0.5% NH₃—H₂O) with MeCN from 0-50%, 25 mL/min, 220 nm) to afford [(2R,5S)-5-(1,3-benzothiazol-5-yl)-2-methyl-piperazin-1-yl]-[1-(trifluoromethyl)cyclopropyl]methanone (40 mg, crude) as a yellow oil.

### Step 2: Synthesis of tert-butyl N-[5-[[2-[(2S,5R)-2-(1,3-benzothiazol-5-yl)-5-methyl-4-[1-(trifluoromethyl)cyclopropanecarbonyl]piperazin-1-yl]-2-oxo-acetyl]amino]-3-cyclopropyl-2-pyridyl]-N-tert-butoxycarbonyl-carbamate

To a mixture of [2-[[6-[bis(tert-butoxycarbonyl)amino]-5-cyclopropyl-3-pyridyl]amino]-2-oxo-acetyl]oxysodium (40 mg, 0.0902 mmol), [(2R,5S)-5-(1,3-benzothiazol-5-yl)-2-methyl-piperazin-1-yl]-[1-(trifluoromethyl)cyclopropyl]methanone (33.0 mg, 0.0893 mmol), HATU (40.0 mg, 0.105 mmol) and DMF (3 mL) was added DIPEA (0.1 mL, 0.612 mmol) and the mixture was stirred at 20° C. for 2 hours. The resulting mixture was quenched by addition of water (10 mL) and extracted with EtOAc (10 mL*3). The combined organic layer was washed with saturated NH₄Cl aqueous solution (10 mL*2), brine (10 mL), dried over anhydrous Na₂SO₄, filtered and concentrated under reduced pressure. The residue was purified by flash chromatography (ISCO®; 24 g AgelaFlash® Silica Flash Column, petroleum ether/EtOAc with EtOAc from 0˜70%, Flow Rate: 30 mL/min) to afford tert-butyl N-[5-[[2-[(2S,5R)-2-(1,3-benzothiazol-5-yl)-5-methyl-4-[1-(trifluoromethyl)cyclopropanecarbonyl]piperazin-1-yl]-2-oxo-acetyl]amino]-3-cyclopropyl-2-pyridyl]-N-tert-butoxycarbonyl-carbamate (40 mg, 57.4% yield) as yellow oil.

### Step 3: Synthesis of N-(6-amino-5-cyclopropyl-3-pyridyl)-2-[(2S,5R)-2-(1,3-benzothiazol-5-yl)-5-methyl-4-[1-(trifluoromethyl)cyclopropanecarbonyl]piperazin-1-yl]-2-oxo-acetamide (Compound 141)

A mixture of tert-butyl N-[5-[[2-[(2S,5R)-2-(1,3-benzothiazol-5-yl)-5-methyl-4-[1-(trifluoromethyl)cyclopropanecarbonyl]piperazin-1-yl]-2-oxo-acetyl]amino]-3-cyclopropyl-2-pyridyl]-N-tert-butoxycarbonyl-carbamate (30.0 mg, 0.0388 mmol), TFA (0.2 mL, 2.60 mmol) and DCM (2 mL) was stirred at 20° C. for 2 hours. The resulting mixture was concentrated under reduced pressure. The residue was purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %); Mobile phase B: MeCN; Gradient: B from 52% to 82% in 9.5 min, hold 100% B for 2.5 min; Flow Rate: 25 mL/min; Column Temperature: 30° C.; Wavelength: 220 nm, 254 nm) to afford N-(6-amino-5-cyclopropyl-3-pyridyl)-2-[(2S,5R)-2-(1,3-benzothiazol-5-yl)-5-methyl-4-[1-(trifluoromethyl)cyclopropanecarbonyl]piperazin-1-yl]-2-oxo-acetamide (8 mg, 36.0% yield) as white solid. ¹H NMR (400 MHz, methanol-d₄) δ ppm 9.07-9.30 (m, 1H), 7.81-8.11 (m, 3H), 7.19-7.55 (m, 2H), 5.07-6.01 (m, 1H), 3.92-4.29 (m, 3H), 3.36-3.57 (m, 1H), 1.59 (br s, 1H), 1.04-1.40 (m, 6H), 0.66-0.99 (m, 4H), 0.50 (br d, J=4.5 Hz, 1H), −0.01-0.32 (m, 1H); LCMS (ESI) [M+H]⁺ m/z: calcd 573.2, found 573.2; HPLC:100%®220 nm, 100%®254 nm; 100% ee.

### Example 3. Compound 53 N-(6-amino-5-methyl-3-pyridyl)-2-[(2R,5R)-5-methyl-4-(1-methylcyclopropanecarbonyl)-2-(2-thienyl)piperazin-1-yl]-2-oxo-acetamide

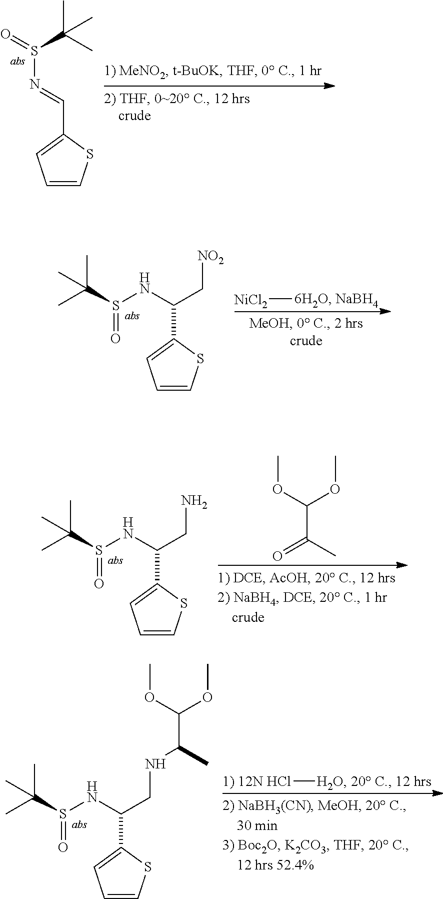

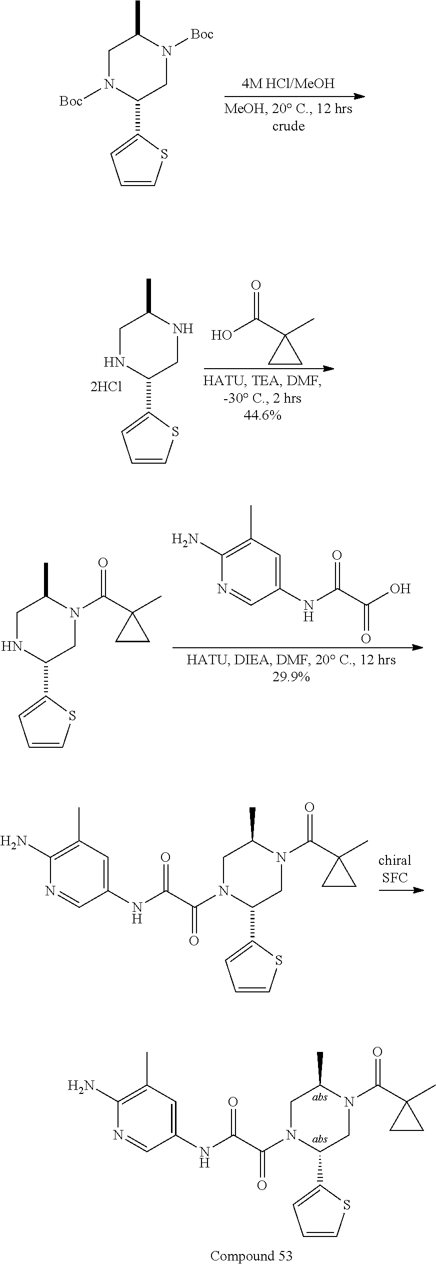

### Step 1: Synthesis of 2-methyl-N-[2-nitro-1-(2-thienyl)ethyl]propane-2-sulfinamide

A mixture of 1M t-BuOK/THF (90 mL, 90.0 mmol) and THF (100 mL) was cooled to 0° C. then the nitromethane (20 mL, 0.370 mol) was added and the solution was stirred at 0° C. for 1 hour. The a solution of (NE)-2-methyl-N-(2-thienylmethylene)propane-2-sulfinamide (5 g, 23.2 mmol) in THF (30 mL) was added to the mixture at 0° C. and the mixture was stirred at 20° C. for 12 hours. The resulting mixture was concentrated under reduced pressure to give the residue, which was purified by flash chromatography (ISCO®; 80 g AgelaFlash® Silica Flash Column, DCM/MeOH with MeOH from 0˜15%, flow rate=30 mL/min, 254 nm) to afford 2-methyl-N-[2-nitro-1-(2-thienyl)ethyl]propane-2-sulfinamide(4 g, crude) as yellow oil.

LCMS (ESI) [M+H]⁺ m/z: calcd 277.1, found 277.0.

### Step 2: Synthesis of N-[2-amino-1-(2-thienyl)ethyl]-2-methyl-propane-2-sulfinamide

To a mixture of 2-methyl-N-[2-nitro-1-(2-thienyl)ethyl]propane-2-sulfinamide (4 g, 14.5 mmol), NiCl2-6H₂O (3.8 g, 16.0 mmol) in MeOH (40 mL) was added NaBH4 (2.8 g, 74.0 mmol) at 0° C. The resulting mixture was stirred at 0° C. for 2 hours. The resulting mixture was filtered and concentrated under reduced pressure to give N-[2-amino-1-(2-thienyl)ethyl]-2-methyl-propane-2-sulfinamide (3.5 g, crude) as brown oil, which was used to the next step with further purification. LCMS (ESI) [M+H]⁺ m/z: calcd 247.1, found 247.1

### Step 3: Synthesis of N-[2-[(2,2-dimethoxy-1-methyl-ethyl)amino]-1-(2-thienyl)ethyl]-2-methyl-propane-2-sulfinamide

A mixture of N-[2-amino-1-(2-thienyl)ethyl]-2-methyl-propane-2-sulfinamide (3.50 g, 14.2 mmol), 1,1-dimethoxypropan-2-one (4.2 g, 35.6 mmol), DCE (30 mL) and AcOH (0.8 mL, 14.0 mmol) was stirred at 20° C. for 12 hours. NaBH4 (1.6 g, 42.3 mmol) was added at 0° C. The resulting mixture was stirred at 0° C. for 1 hour. The resulting mixture was quenched by addition of water (100 mL) and extracted with DCM (100 mL*3). The combined organic layer was washed with saturated NH₄Cl aqueous solution (100 mL), brine (100 mL), dried over anhydrous Na₂SO₄, filtered and concentrated under reduced pressure to give N-[2-[(2,2-dimethoxy-1-methyl-ethyl)amino]-1-(2-thienyl)ethyl]-2-methyl-propane-2-sulfinamide (4 g, crude) as brown oil. LCMS (ESI) [M+H]⁺ m/z: calcd 349.2, found 349.2.

### Step 4: Synthesis of ditert-butyl 2-methyl-5-(2-thienyl)piperazine-1,4-dicarboxylate

A mixture of N-[2-[(2,2-dimethoxy-1-methyl-ethyl)amino]-1-(2-thienyl)ethyl]-2-methyl-propane-2-sulfinamide (4.0 g, 11.5 mmol) in 12N HCl/H₂O (20 mL, 0.240 mol) was stirred at 20° C. for 12 hours. The resulting mixture was concentrated under reduced pressure to give a residue, which was mixed with MeOH (20 mL) and Na₂SO₄ (5 g, 35.2 mmol). Then NaBH3(CN) (740 mg, 11.8 mmol) was added at 0° C. slowly, and the mixture was stirred at 0° C. for 30 minutes. The resulting mixture was concentrated under reduced pressure. The residue was mixed with THF (20 mL), (Boc)₂O (7.5 g, 34.4 mmol) and K2CO₃ (4.8 g, 34.7 mmol). The solution was stirred at 20° C. for 12 hours. The resulting mixture was quenched by addition of water (50 mL) and extracted with EtOAc (100 mL*3). The combined organic layer was washed with saturated NH₄Cl aqueous solution (100 mL), brine (100 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜10%, flow rate=30 mL/min, 254 nm) to afford ditert-butyl 2-methyl-5-(2-thienyl)piperazine-1,4-dicarboxylate (2.3 g, 52.4% yield) as colorless oil. ¹H NMR (400 MHz, methanol-d₄) δ ppm 7.32 (br d, J=5.6 Hz, 1H), 6.86-7.10 (m, 2H), 5.15-5.64 (m, 1H), 4.13-4.42 (m, 2H), 3.37-3.80 (m, 2H), 2.94-3.25 (m, 1H), 1.36-1.56 (m, 21H); LCMS (ESI) [M+H]⁺ m/z: calcd 393.2, found 271.0 (Boc and t-Bu cleaved mass).

### Step 5: Synthesis of 2-methyl-5-(2-thienyl)piperazine

A mixture of ditert-butyl 2-methyl-5-(2-thienyl)piperazine-1,4-dicarboxylate (500 mg, 1.31 mmol) and 4M HCl/MeOH (4 mL, 16.0 mmol) in MeOH (4 mL) was stirred at 20° C. for 12 hours. The resulting mixture was concentrated under reduced pressure to give 2-methyl-5-(2-thienyl)piperazine (280 mg, crude, 2HCl) as white solid. ¹H NMR (400 MHz, methanol-d₄) δ ppm 7.68 (d, J=5.0 Hz, 1H), 7.50 (br d, J=3.3 Hz, 1H), 7.20 (dd, J=5.1, 3.7 Hz, 1H), 5.12 (br dd, J=12.3, 3.4 Hz, 1H), 3.95 (br d, J=3.3 Hz, 1H), 3.82-3.91 (m, 1H), 3.72-3.81 (m, 2H), 3.41-3.53 (m, 1H), 1.51 (d, J=6.6 Hz, 3H).

### Step 6: Synthesis of (1-methylcyclopropyl)-[2-methyl-5-(2-thienyl)piperazin-1-yl]methanone

To a mixture of 2-methyl-5-(2-thienyl)piperazine (260 mg, 1.02 mmol, 2HCl) in DMF (3 mL) was added HATU (390 mg, 1.03 mmol) and TEA (0.650 mL, 4.66 mmol). Then 1-methylcyclopropanecarboxylic acid (80 mg, 0.799 mmol) was added at −30° C. The resulting mixture was stirred at −30° C. for 2 hours. The mixture was concentrated under reduced pressure to give a crude product, which was purified by flash chromatography (Column: SepaFlash® Spherical C18, 40 g, 40-60 m, 120 Å; MeCN/water (0.05 v % NH₃—H₂O) with MeCN from 0-50%, 25 mL/min, 220 nm) to give (1-methylcyclopropyl)-[2-methyl-5-(2-thienyl)piperazin-1-yl]methanone (120 mg, 44.6% yield) as white solid. LCMS (ESI) [M+H]⁺ m/z: calcd 265.1, found 265.1.

### Step 7: Synthesis of N-(6-amino-5-methyl-3-pyridyl)-2-[(5R)-5-methyl-4-(1-methylcyclopropanecarbonyl)-2-(2-thienyl)piperazin-1-yl]-2-oxo-acetamide

A mixture of (1-methylcyclopropyl)-[(2R)-2-methyl-5-(2-thienyl)piperazin-1-yl]methanone (120 mg, 0.454 mmol), 2-[(6-amino-5-methyl-3-pyridyl)amino]-2-oxo-acetic acid (95 mg, 0.487 mmol), HATU (210 mg, 0.552 mmol) and DIPEA (0.25 mL, 1.44 mmol) in DMF (5 mL) was stirred at 20° C. for 12 hours. The mixture was concentrated under reduced pressure to give a crude product, which was purified by preparative HPLC (Instrument: Gilson GX-281 Liquid Handler, Gilson 322 Pump, Gilson 156 UV Detector; Column: Phenomenex Gemini-NX 80*40 mm*3 m; Mobile phase A: H₂O with 0.05% NH₃—H₂O (v %); Mobile phase B: MeCN; Gradient: B from 18% to 48% in 9.5 min, hold 100% B for 2 min; Flow Rate: 25 mL/min; Column Temperature: 30° C.; Wavelength: 220 nm, 254 nm) to afford N-(6-amino-5-methyl-3-pyridyl)-2-[(5R)-5-methyl-4-(1-methylcyclopropanecarbonyl)-2-(2-thienyl)piperazin-1-yl]-2-oxo-acetamide (60 mg, 29.9% yield) as a white solid. LCMS (ESI) [M+H]⁺ m/z: calcd 442.2, found 442.2; HPLC: 99.05% at 254 nm.

### Step 8: Synthesis of N-(6-amino-5-methyl-3-pyridyl)-2-[(2R,5R)-5-methyl-4-(1-methylcyclopropanecarbonyl)-2-(2-thienyl)piperazin-1-yl]-2-oxo-acetamide (Compound 53)

N-(6-amino-5-methyl-3-pyridyl)-2-[(5R)-5-methyl-4-(1-methylcyclopropanecarbonyl)-2-(2-thienyl)piperazin-1-yl]-2-oxo-acetamide (50 mg, 0.113 mmol) was purified by chiral SFC (Instrument: Berger, Multigr AM-II; Column: Daicel Chiralpak IC 250 mm×30 mm×10 m; Mobile phase: supercritical C₀₂/MeOH (0.1% NH₃—H₂O, v %)=55/45; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to Compound 53 (peak 2, retention time: 5.710 min).

Compound 53: N-(6-amino-5-methyl-3-pyridyl)-2-[(2R,5R)-5-methyl-4-(1-methylcyclopropanecarbonyl)-2-(2-thienyl)piperazin-1-yl]-2-oxo-acetamide (34 mg, single known enantiomer with trans relative chemistry, peak 2, retention time: 5.710 min, white solid). ¹H NMR (400 MHz, methanol-d₄) δ ppm 7.99-8.18 (m, 1H), 7.56-7.74 (m, 1H), 7.31-7.45 (m, 1H), 6.88-7.16 (m, 2H), 5.93-6.10 (m, 1H), 4.71-4.89 (m, 2H), 3.96-4.32 (m, 1H), 3.34-3.65 (m, 1H), 3.13 (br d, J=12.5 Hz, 1H), 2.14 (s, 3H), 1.40 (br dd, J=16.9, 5.9 Hz, 2H), 1.25 (br s, 4H), 0.78-1.01 (m, 2H), 0.63 (br d, J=18.1 Hz, 2H); LCMS (ESI) [M+H]⁺ m/z: calcd 442.2, found 442.2; HPLC: 100% at 220 nm, 100% at 254 nm; 100% ee.

### Example 4. Compound 93 N-(6-amino-5-methyl-3-pyridyl)-2-[(2S,5R)-2-(1,3-benzothiazol-5-yl)-5-methyl-4-(2-methylpropanoyl)piperazin-1-yl]-2-oxo-acetamide and N-(6-amino-5-methyl-3-pyridyl)-2-[(2S,5R)-2-(1,3-benzothiazol-5-yl)-5-methyl-4-(2-methylpropanoyl)piperazin-1-yl]-2-oxo-acetamide

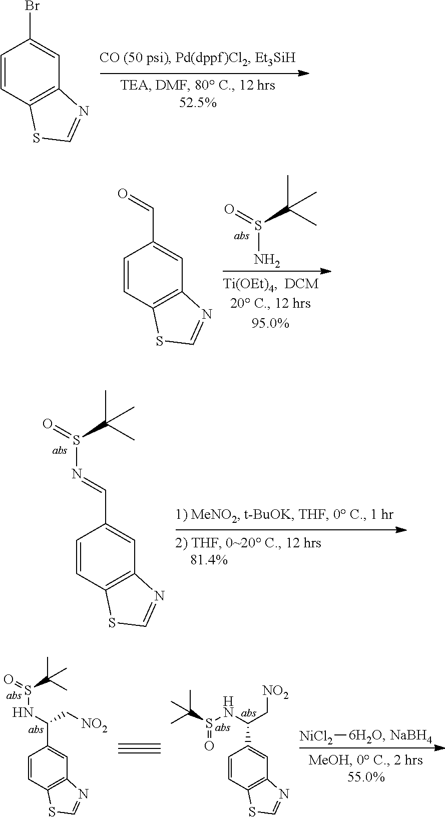

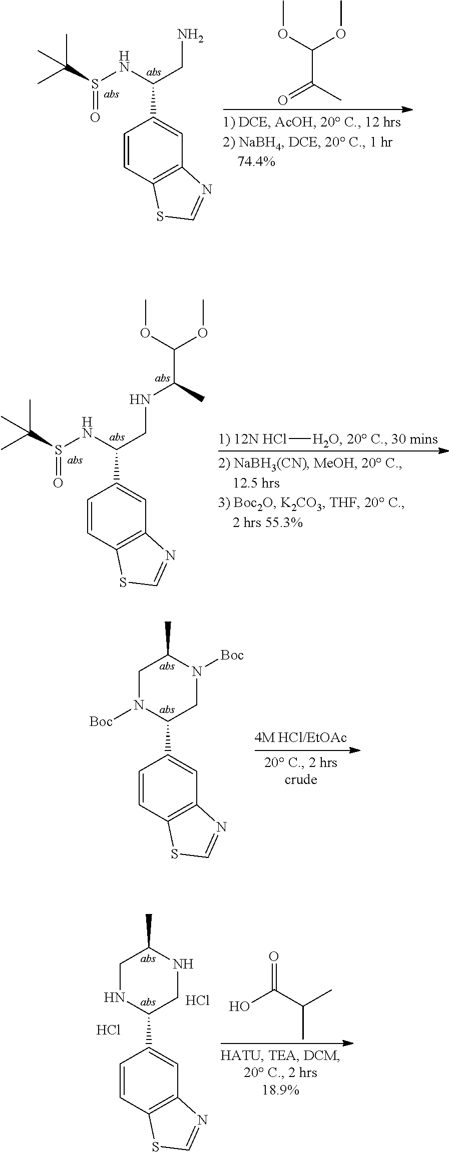

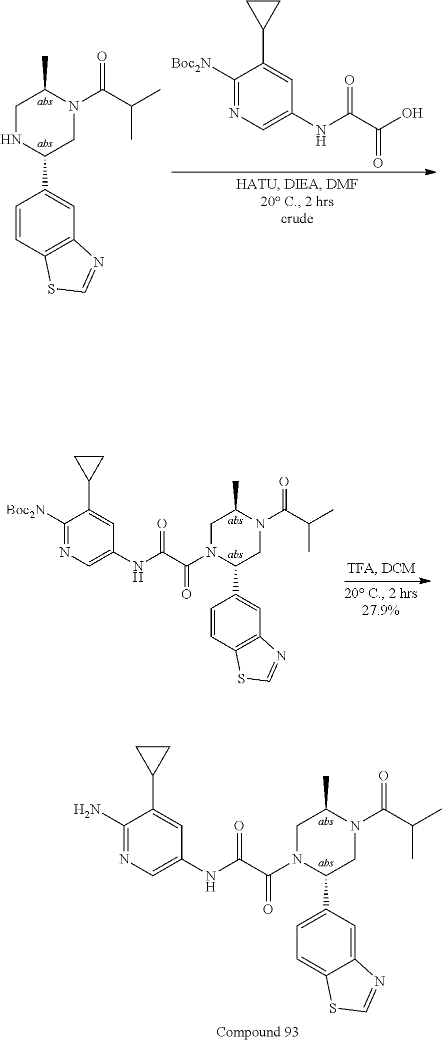

### Step 1: Synthesis of 1,3-benzothiazole-5-carbaldehyde

A mixture of 5-bromo-1,3-benzothiazole (5 g, 23.36 mmol), Pd(dppf)C₁₂ (875 mg, 1.20 mmol), TEA (7.78 g, 76.9 mmol), TES (8.57 g, 73.7 mmol) and DMF (40 mL) was stirred at 80° C. for 12 hours under CO (50 psi). The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 40 g AgelaFlash®Silica Flash Column, petroleum ether/EtOAc with EtOAc from 0˜42%, Flow Rate: 30 mL/min) to affor1,3-benzothiazole-5-carbaldehyde (2 g, 52.5% yield) as white solid. LCMS (ESI) [M+H]⁺ m/z: calcd 164.0; found 164.0.

### Step 2: Synthesis of (NZ)—N-(1,3-benzothiazol-5-ylmethylene)-2-methyl-propane-2-sulfinamide

A mixture of 1,3-benzothiazole-5-carbaldehyde (2 g, 12.26 mmol), 2-methylpropane-2-sulfinamide (2.50 g, 20.6 mmol), Ti(OEt)₄ (8.75 g, 38.4 mmol) and DCM (50 mL) was stirred at 20° C. for 12 hours. The resulting mixture was quenched by addition of water (100 mL) and extracted with DCM(100 mL*3). The combined organic layer was washed with saturated NH₄Cl aqueous solution (100 mL*2), brine (100 mL), dried over anhydrous Na₂SO₄, filtered and concentrated under reduced pressure to give (NZ)—N-(1,3-benzothiazol-5-ylmethylene)-2-methyl-propane-2-sulfinamide (3.1 g, 95.0% yield) as yellow solid.

LCMS (ESI) [M+H]⁺ m/z: calcd 267.0; found 267.0.

### Step 3: Synthesis of N-[(1R)-1-(1,3-benzothiazol-5-yl)-2-nitro-ethyl]-2-methyl-propane-2-sulfinamide

A mixture of potassium; 2-methylpropan-2-olate (1 M, 55 mL) and THF (50 mL) was cooled to 0° C. and then nitromethane (12 mL, 0.222 mol) was added. The mixture was stirred at 0° C. for 1 hour and (NZ)—N-(1,3-benzothiazol-5-ylmethylene)-2-methyl-propane-2-sulfinamide (3.00 g, 11.3 mmol) was added at 0° C. The mixture was stirred at 20° C. for 11 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 40 g AgelaFlash® Silica Flash Column, EtOAc/MeOH with MeOH from 0˜10%, Flow Rate: 30 mL/min, 254 nm) to afford N-[(1R)-1-(1,3-benzothiazol-5-yl)-2-nitro-ethyl]-2-methyl-propane-2-sulfinamide (3 g, 81.4% yield) as yellow oil.

### Step 4: Synthesis of N-[(1R)-2-amino-1-(1,3-benzothiazol-5-yl)ethyl]-2-methyl-propane-2-sulfinamide

A mixture of N-[(1R)-1-(1,3-benzothiazol-5-yl)-2-nitro-ethyl]-2-methyl-propane-2-sulfinamide (3 g, 9.16 mmol), dichloronickel;hexahydrate (2.31 g, 9.71 mmol) and MeOH (20 mL) was cooled to 0° C. NaBH4 (1.73 g, 45.8 mmol) was added at 0° C. and the mixture was stirred at 0° C. for 2 hours. The mixture was filtered and concentrated under reduced pressure to afford N-[(1R)-2-amino-1-(1,3-benzothiazol-5-yl)ethyl]-2-methyl-propane-2-sulfinamide (1.5 g, 55.0% yield) as a yellow oil.

### Step 5: Synthesis of N-[(1S)-1-(1,3-benzothiazol-5-yl)-2-[[(1R)-2,2-dimethoxy-1-methyl-ethyl]amino]ethyl]-2-methyl-propane-2-sulfinamide

A mixture of N-[(1S)-2-amino-1-(1,3-benzothiazol-5-yl)ethyl]-2-methyl-propane-2-sulfinamide (1.5 g, 5.04 mmol), Na₂SO₄ (2.55 g, 18.0 mmol), AcOH (1.50 g, 6.15 mmol) and DCE (20 mL) was stirred at 20° C. for 12 hours. The NaBH4 (600 mg, 15.9 mmol) was added and the mixture was stirred at 20° C. for 1 hour. The resulting mixture was quenched by addition of water (50 mL) and extracted with DCM (100 mL*3). The combined organic layer was washed with saturated NH₄Cl aqueous solution (50 mL*2), brine (50 mL), dried over anhydrous Na₂SO₄, filtered and concentrated under reduced pressure to give N-[(1S)-1-(1,3-benzothiazol-5-yl)-2-[[(1R)-2,2-dimethoxy-1-methyl-ethyl]amino]ethyl]-2-methyl-propane-2-sulfinamide (1.5 g, 74.4% yield) as a yellow oil.

### Step 6: Synthesis of ditert-butyl (2S,5R)-2-(1,3-benzothiazol-5-yl)-5-methyl-piperazine-1,4-dicarboxylate

A mixture of N-[(1S)-1-(1,3-benzothiazol-5-yl)-2-[[(1R)-2,2-dimethoxy-1-methyl-ethyl]amino]ethyl]-2-methyl-propane-2-sulfinamide (1.5 g, 3.75 mmol) and 12N HCl/H₂O (10 mL, 0.12 mol) was stirred at 20° C. for 30 minutes. The mixture was concentrated under reduced pressure and the residue was mixed with sodium;cyanoboranuide (100 mg, 1.59 mmol), Na₂SO₄ (1.80 g, 12.7 mmol) and MeOH (20 mL) and the mixture was stirred at 20° C. for 12.5 hours. Then the mixture was concentrated under reduced pressure and the residue was mixed with tert-butoxycarbonyl tert-butyl carbonate (3 mL, 13.1 mmol), K2CO₃ (1.59 g, 11.5 mmol), THF (30 mL) and H₂O (10 mL) and the mixture was stirred at 20° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 12 g AgelaFlash® Silica Flash Column, petroleum ether/EtOAc with EtOAc from 0˜30%, Flow Rate: 30 mL/min, 254 nm) to afford ditert-butyl (2S,5R)-2-(1,3-benzothiazol-5-yl)-5-methyl-piperazine-1,4-dicarboxylate (900 mg, 55.3% yield) as a yellow oil.

### Step 7: Synthesis of 5-[(2S,5R)-5-methylpiperazin-2-yl]-1,3-benzothiazole

A mixture of ditert-butyl (2S,5R)-2-(1,3-benzothiazol-5-yl)-5-methyl-piperazine-1,4-dicarboxylate (900 mg, 2.08 mmol) and 4M HCl/MeOH (5 mL, 20 mmol) was stirred at 20° C. for 2 hours. Then the mixture was concentrated under reduced pressure to give 5-[(2S,5R)-5-methylpiperazin-2-yl]-1,3-benzothiazole (500 mg, crude, 2HCl) as yellow solid.

### Step 8: Synthesis of 1-[(2R,5S)-5-(1,3-benzothiazol-5-yl)-2-methyl-piperazin-1-yl]-2-methyl-propan-1-one

A mixture of 5-[(2S,5R)-5-methylpiperazin-2-yl]-1,3-benzothiazole (160 mg, 0.522 mmol, 2HCl), 2-methylpropanoic acid (50 mg, 0.568 mmol), HATU (290 mg, 0.763 mmol) and DCM (3 mL) was added DIPEA (0.4 mL, 2.30 mmol) at 0° C. then the mixture was stirred at 20° C. for 2 hours. The mixture was purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %);Mobile phase B: MeCN; Gradient: B from 15% to 45% in 9.5 min, hold 100% B for 2.5 min; Flow Rate: 25 mL/min; Column Temperature: 30° C.; Wavelength: 220 nm, 254 nm) to afford 1-[(2R,5S)-5-(1,3-benzothiazol-5-yl)-2-methyl-piperazin-1-yl]-2-methyl-propan-1-one (30 mg, 18.9% yield) as yellow oil. ¹H NMR (400 MHz, DMSO-d₆) δ ppm 9.35 (s, 1H), 8.03-8.18 (m, 2H), 7.57 (br d, J=8.3 Hz, 1H), 3.75-4.78 (m, 5H), 2.65-2.90 (m, 2H), 2.54-2.60 (m, 1H), 1.26 (br s, 3H), 0.90 (br d, J=6.8 Hz, 6H), 0.00-0.00 (m, 1H).

### Step 9: Synthesis of tert-butyl N-[5-[[2-[(2S,5R)-2-(1,3-benzothiazol-5-yl)-5-methyl-4-(2-methylpropanoyl)piperazin-1-yl]-2-oxo-acetyl]amino]-3-cyclopropyl-2-pyridyl]-N-tert-butoxycarbonyl-carbamate

A mixture of [2-[[6-[bis(tert-butoxycarbonyl)amino]-5-cyclopropyl-3-pyridyl]amino]-2-oxo-acetyl]oxysodium (25 mg, 56.4 mol), 1-[(2R,5S)-5-(1,3-benzothiazol-5-yl)-2-methyl-piperazin-1-yl]-2-methyl-propan-1-one (25.0 mg, 82.4 μmol), HATU (25.0 mg, 65.8 mol) and DMF (3 mL) was added DIPEA (49.5 mg, 0.383 mmol) and the mixture was stirred at 20° C. for 2 hours. The resulting mixture was quenched by addition of water (10 mL) and extracted with EtOAc (10 mL*3). The combined organic layer was washed with saturated NH₄Cl aqueous solution (10 mL*2), brine (10 mL), dried over anhydrous Na₂SO₄, filtered and concentrated under reduced pressure to give a residue which was purified by flash chromatography (ISCO®; 4 g AgelaFlash® Silica Flash Column, petroleum ether/EtOAc with EtOAc from 0˜50%, Flow Rate: 30 mL/min, 254 nm) to afford tert-butyl N-[5-[[2-[(2S,5R)-2-(1,3-benzothiazol-5-yl)-5-methyl-4-(2-methylpropanoyl)piperazin-1-yl]-2-oxo-acetyl]amino]-3-cyclopropyl-2-pyridyl]-N-tert-butoxycarbonyl-carbamate (50 mg, crude) as yellow oil.

### Step 10: Synthesis of N-(6-amino-5-cyclopropyl-3-pyridyl)-2-[(2S,5R)-2-(1,3-benzothiazol-5-yl)-5-methyl-4-(2-methylpropanoyl)piperazin-1-yl]-2-oxo-acetamide (Compound 93)

A mixture of tert-butyl N-[5-[[2-[(2S,5R)-2-(1,3-benzothiazol-5-yl)-5-methyl-4-(2-methylpropanoyl)piperazin-1-yl]-2-oxo-acetyl]amino]-3-cyclopropyl-2-pyridyl]-N-tert-butoxycarbonyl-carbamate (50.0 mg, 70.7 mol), TFA (1 mL, 13.0 mmol) and DCM (2 mL) and the mixture was stirred at 20° C. for 2 hours. The resulting mixture purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %);Mobile phase B: MeCN; Gradient: B from 19% to 46% in 9.5 min, hold 100% B for 2.5 min; Flow Rate: 25 mL/min; Column Temperature: 30° C.; Wavelength: 220 nm, 254 nm) to afford N-(6-amino-5-cyclopropyl-3-pyridyl)-2-[(2S,5R)-2-(1,3-benzothiazol-5-yl)-5-methyl-4-(2-methylpropanoyl)piperazin-1-yl]-2-oxo-acetamide (10 mg, 27.9% yield) as white solid. ¹H NMR (400 MHz, methanol-d₄) δ ppm 8.93-9.33 (m, 1H), 7.83-8.11 (m, 2H), 6.76-7.83 (m, 3H), 5.51-5.93 (m, 1H), 4.98-5.34 (m, 1H), 4.51-4.61 (m, 1H), 4.06-4.31 (m, 1H), 3.79-4.03 (m, 1H), 3.26-3.51 (m, 1H), 2.24-3.03 (m, 1H), 1.39-1.67 (m, 1H), 1.24-1.34 (m, 2H), 1.18 (dd, J=6.4, 3.6 Hz, 1H), 1.02 (d, J=6.5 Hz, 2H), 0.73-0.96 (m, 5H), 0.61 (d, J=6.5 Hz, 1H), 0.42-0.54 (m, 2H); LCMS (ESI) [M+H]⁺ m/z: calcd 507.1, found 507.1; HPLC 100%®254 nm; 99.8% ee.

### Example 5. Compound 31 N-(6-amino-5-methyl-3-pyridyl)-2-[(2S,5R)-5-methyl-4-(1-methylcyclopropanecarbonyl)-2-(3-thienyl)piperazin-1-yl]-2-oxo-acetamide

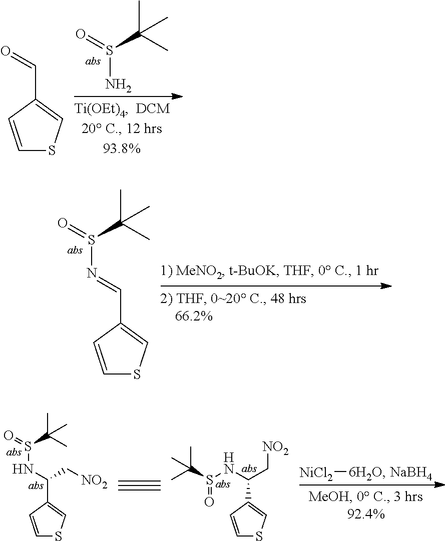

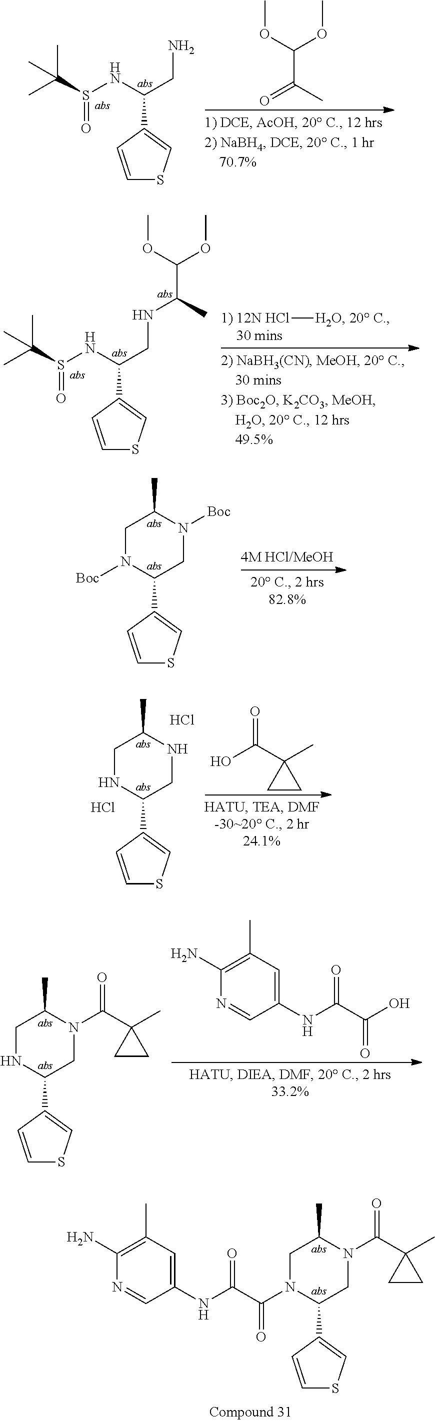

### Step 1: Synthesis of (NZ)-2-methyl-N-(3-thienylmethylene)propane-2-sulfinamide

A mixture of thiophene-3-carbaldehyde (10 g, 89.2 mmol), 2-methylpropane-2-sulfinamide (13 g, 0.107 mol), Ti(OEt)₄ (0.239 mol, 50 mL) and DCM (50 mL) was stirred at 20° C. for 12 hours. The resulting mixture was quenched by addition of water (100 mL) and extracted with DCM (100 mL*3). The combined organic layer was washed with saturated NH₄Cl aqueous solution (100 mL*2), brine (100 mL), dried over anhydrous Na₂SO₄, filtered and concentrated under reduced pressure to give (NZ)-2-methyl-N-(3-thienylmethylene)propane-2-sulfinamide (18 g, 93.8% yield) as yellow solid.

### Step 2: Synthesis of 2-methyl-N-[(1S)-2-nitro-1-(3-thienyl)ethyl]propane-2-sulfinamide

A mixture of 1M t-BuOK/THF (190 mL, 0.19 mol) and THF (100 mL) was cooled to 0° C. then the nitromethane (40 mL, 0.740 mol) was added and the mixture was stirred at 0° C. for 1 hour. (NE)-2-methyl-N-(3-thienylmethylene)propane-2-sulfinamide (10 g, 46.4 mmol) was added at 0° C. and the mixture was stirred at 20° C. for 48 hours. The mixture was concentrated under reduced pressure to give a residue which was purified by flash chromatography (ISCO®; 80 g AgelaFlash®Silica Flash Column, DCM/MeOH with MeOH from 0˜20%, Flow Rate: 30 mL/min, 254 nm) to afford 2-methyl-N-[(1S)-2-nitro-1-(3-thienyl)ethyl]propane-2-sulfinamide (8.5 g, 66.2% yield) as yellow oil.

### Step 3: Synthesis of N-[(1S)-2-amino-1-(3-thienyl)ethyl]-2-methyl-propane-2-sulfinamide

A mixture of 2-methyl-N-[(1S)-2-nitro-1-(3-thienyl)ethyl]propane-2-sulfinamide (8.5 g, 30.8 mmol), dichloronickel;hexahydrate (8.1 g, 34.1 mmol) and MeOH (20 mL) was cooled to 0° C. then NaBH4 (5.8 g, 0.153 mol) was added slowly and the mixture was stirred at 0° C. for 3 hours. The mixture was concentrated under reduced pressure. The residue was triturated with DCM (200 mL) and the filtrate was concentrated under reduced pressure to give N-[(1S)-2-amino-1-(3-thienyl)ethyl]-2-methyl-propane-2-sulfinamide (7 g, 92.4% yield) as black oil.

### Step 4: Synthesis of N-[(1S)-2-[[(1R)-2,2-dimethoxy-1-methyl-ethyl]amino]-1-(3-thienyl)ethyl]-2-methyl-propane-2-sulfinamide

A mixture of N-[(1S)-2-amino-1-(3-thienyl)ethyl]-2-methyl-propane-2-sulfinamide (7 g, 28.4 mmol), 1,1-dimethoxypropan-2-one (7 g, 59.3 mmol), Na₂SO₄ (14.4 g, 0.101 mol), AcOH (1.5 g, 34.7 mmol) and DCE (20 mL) was stirred at 20° C. for 12 hours. The NaBH4 (3.38 g, 89.3 mmol) was added and the mixture was stirred at 20° C. for 1 hour. The resulting mixture was quenched by addition of water (50 mL) and extracted with DCM (100 mL*3). The combined organic layer was washed with saturated NH₄Cl aqueous solution (50 mL*2), brine (50 mL), dried over anhydrous Na₂SO₄, filtered and concentrated under reduced pressure to give N-[(1S)-2-[[(1R)-2,2-dimethoxy-1-methyl-ethyl]amino]-1-(3-thienyl)ethyl]-2-methyl-propane-2-sulfinamide (7 g, 70.7% yield) as black oil.

### Step 5: Synthesis of ditert-butyl (2R,5S)-2-methyl-5-(3-thienyl)piperazine-1,4-dicarboxylate

A mixture of N-[(1S)-2-[[(1R)-2,2-dimethoxy-1-methyl-ethyl]amino]-1-(3-thienyl)ethyl]-2-methyl-propane-2-sulfinamide (7 g, 20.1 mmol) and 12N HCl/H₂O (20 mL, 0.240 mmol) was stirred at 20° C. for 30 minutes. The mixture was concentrated under reduced pressure and the residue was mixed with NaBH3(CN) (1.26 g, 20.0 mmol), Na₂SO₄ (9.63 g, 67.8 mmol) and MeOH (20 mL) and the mixture was stirred at 0° C. for 30 minutes. The mixture was concentrated under reduced pressure and the residue was mixed with tert-butoxycarbonyl tert-butyl carbonate (69.9 mmol, 16.0 mL), K₂CO₃ (8.51 g, 61.6 mmol), H₂O (5 mL) and the mixture was stirred at 20° C. for 12 hours. The resulting mixture was quenched by addition of water (50 mL) and extracted with EtOAc (100 mL*3). The combined organic layer was washed with saturated NH₄Cl aqueous solution (50 mL*2), brine (50 mL), dried over anhydrous Na₂SO₄, filtered and concentrated under reduced pressure. The crude product was purified by flash chromatography (ISCO®; 12 g AgelaFlash® Silica Flash Column, petroleum ether/EtOAc with EtOAc from 0˜30%, Flow Rate: 30 mL/min, 254 nm) to afford ditert-butyl (2R,5S)-2-methyl-5-(3-thienyl)piperazine-1,4-dicarboxylate (3.8 g, 49.5% yield) as yellow oil.

### Step 6: Synthesis of (2R,5S)-2-methyl-5-(3-thienyl)piperazine

A mixture of ditert-butyl (2R,5S)-2-methyl-5-(3-thienyl)piperazine-1,4-dicarboxylate (3.8 g, 9.93 mmol) and 4M HCl/MeOH (10 mL, 40 mmol) was stirred at 20° C. for 12 hours. The mixture was filtered and concentrated under reduced pressure to give (2R,5S)-2-methyl-5-(3-thienyl)piperazine (2.1 g, 82.8% yield, 2HCl) as yellow solid.

### Step 7: Synthesis of (1-methylcyclopropyl)-[(2R,5S)-2-methyl-5-(3-thienyl)piperazin-1-yl]methanone

A mixture of (2R,5S)-2-methyl-5-(3-thienyl)piperazine (200 mg, 0.784 mmol, 2HCl), 1-methylcyclopropanecarboxylic acid (60 mg, 0.599 mmol), HATU (48 mg, 1.26 mmol) and DMF (3 mL) was added DIPEA (0.6 mL, 3.44 mmol) at −30° C. and the mixture was stirred at 20° C. for 2 hours. The residue was purified by flash chromatography (Biotage®, Column: SepaFlash® Spherical C18, 25 g, 40-60 μm, 120 Å;MeCN/water (0.5% NH₃—H₂O) with MeCN from 0-55%, 25 mL/min, 220 nm) to give (1-methylcyclopropyl)-[(2R,5S)-2-methyl-5-(3-thienyl)piperazin-1-yl]methanone (50 mg, 24.1% yield) as yellow oil.

### Step 8: Synthesis of N-(6-amino-5-methyl-3-pyridyl)-2-[(2S,5R)-5-methyl-4-(1-methylcyclopropanecarbonyl)-2-(3-thienyl)piperazin-1-yl]-2-oxo-acetamide (Compound 31)

To a mixture of 2-[(6-amino-5-methyl-3-pyridyl)amino]-2-oxo-acetic acid (40 mg, 0.205 mmol), (1-methylcyclopropyl)-[(2R,5S)-2-methyl-5-(3-thienyl)piperazin-1-yl]methanone (50.0 mg, 0.189 mmol), HATU (77 mg, 0.202 mmol) and DMF (2 mL) was added DIPEA (0.2 mL, 1.15 mmol) and the mixture was stirred at 20° C. for 2 hours. The mixture was purified by flash chromatography (Biotage®, Column: SepaFlash® Spherical C18, 25 g,40-60 m, 120 Å; MeCN/water (0.5% NH₃—H₂O) with MeCN from 0-62%, 25 mL/min, 254 nm) to give a crude product (˜80 mg) which was further purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %); Mobile phase B: MeCN; Gradient: B from 20% to 50% in 9.5 min, hold 100% B for 2.5 min; Flow Rate: 25 mL/min; Column Temperature: 30° C.; Wavelength: 220 nm, 254 nm) to afford N-(6-amino-5-methyl-3-pyridyl)-2-[(2S,5R)-5-methyl-4-(1-methylcyclopropanecarbonyl)-2-(3-thienyl)piperazin-1-yl]-2-oxo-acetamide (30 mg, 33.2% yield) as white solid. ¹H NMR (400 MHz, DMSO-d₆) δ ppm 10.24-10.56 (m, 1H), 10.28 (br s, 1H), 8.07 (s, 1H), 7.36-7.66 (m, 2H), 7.31 (br s, 1H), 6.99-7.14 (m, 1H), 5.25-5.70 (m, 2H), 4.49-4.78 (m, 2H), 3.73-4.30 (m, 1H), 3.34 (br s, 2H), 2.93 (br s, 1H), 1.97-2.12 (m, 3H), 1.22-1.33 (m, 3H), 1.13 (d, J=5.0 Hz, 3H), 0.38-0.78 (m, 4H); LCMS (ESI) [M+H]⁺ m/z: calcd 442.2, found 442.2; HPLC: 95.30%®220 nm, 96.81%®254 nm; 94.0% ee.

### Example 6. Compound 138 N-(6-amino-5-methyl-3-pyridyl)-2-[(2S,5R)-5-methyl-2-(3-thienyl)-4-[1-(trifluoromethyl)cyclopropanecarbonyl]piperazin-1-yl]-2-oxo-acetamide

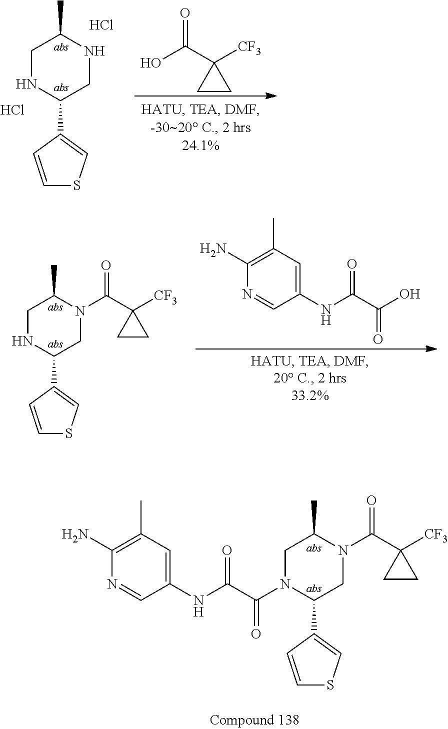

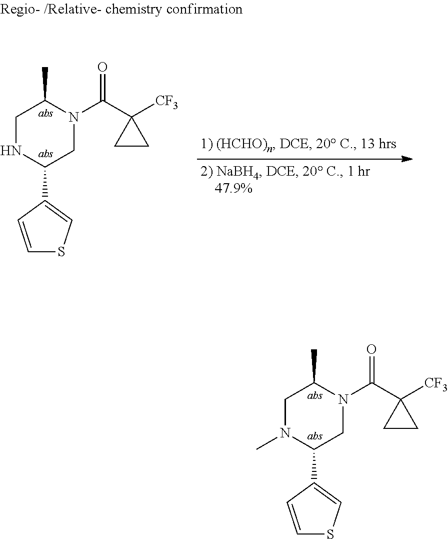 [SMILES: C[C@@H]1CN[C@@H](c2ccsc2)CN1C(=O)C1(C(F)(F)F)CC1 ; C[C@@H]1CN(C)[C@@H](c2ccsc2)CN1C(=O)C1(C(F)(F)F)CC1]

### Step 1: Synthesis of [(2R,5S)-2-methyl-5-(3-thienyl)piperazin-1-yl]-[1-(trifluoromethyl)cyclopropyl]methanone

A mixture of (2R,5S)-2-methyl-5-(3-thienyl)piperazine (500 mg, 1.96 mmol, 2HCl), 1-(trifluoromethyl)cyclopropanecarboxylic acid (230 mg, 1.49 mmol), HATU (1 g, 2.63 mmol) and DCM (10 mL) was added DIPEA (1.5 mL, 8.61 mmol) at −30° C. and then the mixture was stirred at 20° C. for 2 hours. The mixture was purified by flash chromatography (Biotage®, Column: SepaFlash® Spherical C18, 25 g, 40-60 m, 120 Å; MeCN/water (0.5% NH₃—H₂O) with MeCN from 0-55%, 25 mL/min, 220 nm) to give [(2R,5S)-2-methyl-5-(3-thienyl)piperazin-1-yl]-[1-(trifluoromethyl)cyclopropyl]methanone (500 mg, 80.2% yield) as a yellow oil.

### Step 2: Synthesis of N-(6-amino-5-methyl-3-pyridyl)-2-[(2S,5R)-5-methyl-2-(3-thienyl)-4-[1-(trifluoromethyl)cyclopropanecarbonyl]piperazin-1-yl]-2-oxo-acetamide (Compound 138)

To a mixture of 2-[(6-amino-5-methyl-3-pyridyl)amino]-2-oxo-acetic acid (90 mg, 0.461 mmol), [(2R,5S)-2-methyl-5-(3-thienyl)piperazin-1-yl]-[1-(trifluoromethyl)cyclopropyl]methanone (120 mg, 0.377 mol), HATU (180 mg, 0.473 mmol) and DMF (2 mL) was added DIPEA (0.5 mL, 2.87 mmol) and the mixture was stirred at 20° C. for 2 hours. The mixture was purified by (Biotage®; Column: SepaFlash® Spherical C18, 25 g, 40-60 m, 120 Å; MeCN/water (0.5 v % NH₃—H₂O) with MeCN from 0-55%, 25 mL/min, 220 nm) to give a crude product which was purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %); Mobile phase B: MeCN; Gradient: B from 52% to 82% in 9.5 min, hold 100% B for 2.5 min; Flow Rate: 25 mL/min; Column Temperature: 30° C.; Wavelength: 220 nm, 254 nm) to afford N-(6-amino-5-methyl-3-pyridyl)-2-[(2S,5R)-5-methyl-2-(3-thienyl)-4-[1-(trifluoromethyl)cyclopropanecarbonyl]piperazin-1-yl]-2-oxo-acetamide (65 mg, 28.4% yield) as white solid. ¹H NMR (400 MHz, DMSO-d₆) δ ppm 10.25-10.66 (m, 1H), 8.03-8.21 (m, 1H), 7.45-7.61 (m, 2H), 7.29 (br s, 1H), 6.99-7.13 (m, 1H), 5.16-5.81 (m, 2H), 4.73 (br s, 1H), 4.51 (br s, 1H), 3.70-4.22 (m, 1H), 3.45 (br s, 2H), 2.64-3.13 (m, 1H), 2.08 (d, J=5.3 Hz, 3H), 0.92-1.36 (m, 7H); ¹⁹F NMR (376 MHz, DMSO-d₆)₈ ppm -66.377; LCMS (ESI) [M+H]⁺ m/z: calcd 496.4; found 496.4; HPLC: 99.75%®220 nm, 100%®254 nm;

### Step 3: Synthesis of N-(6-amino-5-methyl-3-pyridyl)-2-[(2S,5R)-5-methyl-2-(3-thienyl)-4-[1-(trifluoromethyl)cyclopropanecarbonyl]piperazin-1-yl]-2-oxo-acetamide

A mixture of [(2R,5S)-2-methyl-5-(3-thienyl)piperazin-1-yl]-[1-(trifluoromethyl)cyclopropyl]methanone (60.0 mg, 0.188 mmol) and paraformaldehyde (20 mg, 0.666 mmol) and DCE (10 mL) was stirred at 20° C. for 13 hours. Then NaBH4 (22 mg, 0.582 mmol) was added and the mixture then the mixture was stirred at 20° C. for 1 hour. Then the mixture was purified by flash chromatography (Biotage®, Column: SepaFlash® Spherical C18, 25 g, 40-60 m, 120 Å; MeCN/water (0.5 v % NH₃—H₂O) with MeCN from 0-42%, 25 mL/min, 254 nm) to give [(2R,5S)-2,4-dimethyl-5-(3-thienyl)piperazin-1-yl]-[1-(trifluoromethyl)cyclopropyl]methanone (30 mg, 47.9% yield) as yellow oil. ¹H NMR (400 MHz, methanol-d₄) δ ppm 7.41 (dd, J=4.9, 2.9 Hz, 1H), 7.22 (dd, J=2.8, 1.0 Hz, 1H), 7.00 (dd, J=5.0, 1.3 Hz, 1H), 4.60-4.69 (m, 1H), 4.14 (br d, J=13.8 Hz, 1H), 3.94 (br d, J=3.5 Hz, 1H), 3.77 (dd, J=13.8, 4.5 Hz, 1H), 2.89 (dd, J=12.3, 5.0 Hz, 1H), 2.50 (dd, J=12.4, 4.4 Hz, 1H), 2.18 (s, 3H), 2.00-2.05 (m, 1H), 1.35 (d, J=6.8 Hz, 4H), 0.99-1.33 (m, 5H). The regio-chemistry was confirmed by HMBC and the relative chemistry was confirmed by NOE.

### Example 7. Compound 61 2-oxo-N-(1H-pyrazolo[4,3-c]pyridin-7-yl)-2-[rac-(2S,5R)-2-(1,3-benzothiazol-5-yl)-5-methyl-4-(1-methylcyclopropanecarbonyl)piperazin-1-yl]acetamide

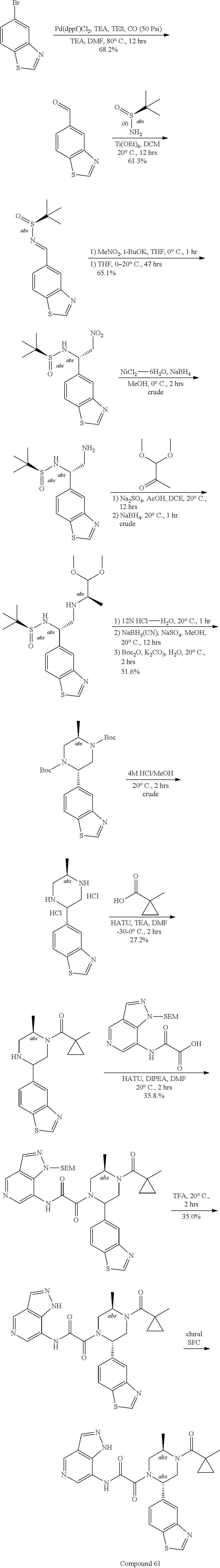

### Step 1: Synthesis of 1,3-benzothiazole-5-carbaldehyde

A mixture of 5-bromo-1,3-benzothiazole (25 g, 0.117 mol), Pd(dppf)C₁₂ (4.37 g, 5.98 mmol), TEA (55 mL, 0.395 mol), TES (43.8 g, 0.376 mol) and DMF (100 mL) was stirred at 80° C. for 12 hours under CO (50 Psi). The resulting mixture was quenched by addition of water (100 mL) and extracted with EtOAc (200 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 80 g AgelaFlash® Silica Flash Column, petroleum ether/EtOAc with EtOAc from 0-.50%, Flow Rate: 30 mL/min, 254 nm) to afford 1,3-benzothiazole-5-carbaldehyde (13 g, 68.2% yield) as yellow solid.

### Step 2: Synthesis of (NZ)—N-(1,3-benzothiazol-5-ylmethylene)-2-methyl-propane-2-sulfinamide

A mixture of 1,3-benzothiazole-5-carbaldehyde (10 g, 61.3 mmol), 2-methylpropane-2-sulfinamide (10 g, 82.5 mmol), ethanolate;titanium(4+) (40 mL, 0.919 mol) and DCM (20 mL) was stirred at 20° C. for 12 hours. The resulting mixture was quenched by addition of water (100 mL) and extracted with DCM (100 mL*3). The combined organic layer was washed with saturated NH₄Cl aqueous solution (100 mL*2), brine (100 mL), dried over anhydrous Na₂SO₄, filtered and concentrated under reduced pressure to give (NZ)—N-(1,3-benzothiazol-5-ylmethylene)-2-methyl-propane-2-sulfinamide (10 g, 61.3% yield) as yellow solid. LCMS (ESI) [M+H]⁺ m/z: calcd 267.1; found 267.0.

### Step 3: Synthesis of N-[(1R)-1-(1,3-benzothiazol-5-yl)-2-nitro-ethyl]-2-methyl-propane-2-sulfinamide

A mixture of 1M t-BuOK/THF (200 mL, 0.200 mol) and THF (100 mL) was cooled to 0° C. Then nitromethane (42 mL, 0.778 mol) was added to the mixture, and the mixture was stirred at 0° C. for 1 hour. (NZ)—N-(1,3-benzothiazol-5-ylmethylene)-2-methyl-propane-2-sulfinamide (10 g, 37.5 mmol) was added to the mixture at 0° C. and the mixture was stirred at 20° C. for 47 hours. The resulting mixture was concentrated under reduced pressure. The residue was purified by flash chromatography (ISCO®; 40 g AgelaFlash® Silica Flash Column, EtOAc/MeOH with MeOH from 0˜10%, Flow Rate: 30 mL/min, 254 nm) to afford N-[(1R)-1-(1,3-benzothiazol-5-yl)-2-nitro-ethyl]-2-methyl-propane-2-sulfinamide (8 g, 65.1% yield) as yellow oil. LCMS (ESI) [M+H]⁺ m/z: calcd 328.1; found 328.1.

### Step 4: Synthesis of N-[(1R)-2-amino-1-(1,3-benzothiazol-5-yl)ethyl]-2-methyl-propane-2-sulfinamide

A mixture of N-[(1R)-1-(1,3-benzothiazol-5-yl)-2-nitro-ethyl]-2-methyl-propane-2-sulfinamide (7.5 g, 22.9 mmol), dichloronickel;hexahydrate (6 g, 25.2 mmol) and MeOH (50 mL) was cooled to 0° C. Then NaBH4 (4.4 g, 0.116 mol) was added slowly and was stirred at 0° C. for 2 hours. The mixture was filtered and concentrated (30° C.) under reduced pressure to give N-[(1R)-2-amino-1-(1,3-benzothiazol-5-yl)ethyl]-2-methyl-propane-2-sulfinamide (7.5 g, crude) as black oil. LCMS (ESI) [M+H]⁺ m/z: calcd 298.1; found 298.1.

### Step 5: Synthesis of N-[(1S)-1-(1,3-benzothiazol-5-yl)-2-[[(1R)-2,2-dimethoxy-1-methyl-ethyl]amino]ethyl]-2-methyl-propane-2-sulfinamide

A mixture of N-[(1S)-2-amino-1-(1,3-benzothiazol-5-yl)ethyl]-2-methyl-propane-2-sulfinamide (7.5 g, 25.2 mmol), 1,1-dimethoxypropan-2-one (8 g, 67.7 mmol), Na₂SO₄ (13 g, 91.5 mmol), AcOH (1.5 g, 30.7 mmol) and DCE (100 mL) was stirred at 20° C. for 12 hours. NaBH4 (3 g, 79.2 mmol) was added to the mixture and the mixture was stirred at 20° C. for 1 hour. The resulting mixture was quenched by addition of water (50 mL) and extracted with DCM (100 mL*3). The combined organic layer was washed with saturated NH₄Cl aqueous solution (50 mL*2), brine (50 mL), dried over anhydrous Na₂SO₄, filtered and concentrated under reduced pressure to give N-[(1S)-1-(1,3-benzothiazol-5-yl)-2-[[(1R)-2,2-dimethoxy-1-methyl-ethyl]amino]ethyl]-2-methyl-propane-2-sulfinamide (11 g, crude) as yellow oil.

### Step 6: Synthesis of ditert-butyl (2S,5R)-2-(1,3-benzothiazol-5-yl)-5-methyl-piperazine-1,4-dicarboxylate

A mixture of N-[(1S)-1-(1,3-benzothiazol-5-yl)-2-[[(1R)-2,2-dimethoxy-1-methyl-ethyl]amino]ethyl]-2-methyl-propane-2-sulfinamide (7.5 g, 18.8 mmol) and 12M HCl/H₂O (20 mL, 0.240 mol) was stirred at 20° C. for 1 hour. The mixture was concentrated under reduced pressure to give mixture, which was added sodium;cyanoboranuide (500 mg, 7.96 mmol), Na₂SO₄ (9 g, 63.4 mmol) and MeOH (20 mL). Then the mixture was stirred at 20° C. for 12 hours. To the mixture was added tert-butoxycarbonyl tert-butyl carbonate (15 mL, 65.4 mmol), K₂CO₃ (8 g, 57.9 mmol) and H₂O (10 mL). The mixture was stirred at 20° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure. The residue was purified by flash chromatography (ISCO®; 12 g AgelaFlash® Silica Flash Column, petroleum ether/EtOAc with EtOAc from 0˜30%, Flow Rate: 30 mL/min, 254 nm) to afford ditert-butyl (2S,5R)-2-(1,3-benzothiazol-5-yl)-5-methyl-piperazine-1,4-dicarboxylate (4.2 g, 51.6% yield) as yellow oil. LCMS (ESI) [M+H]⁺ m/z: calcd 434.2; found 434.2.

### Step 7: Synthesis of 5-[rac-(2S,5R)-5-methylpiperazin-2-yl]-1,3-benzothiazole

A mixture of ditert-butyl rac-(2S,5R)-2-(1,3-benzothiazol-5-yl)-5-methyl-piperazine-1,4-dicarboxylate (4.2 g, 9.69 mmol) and 4M HCl/MeOH (10 mL, 40.0 mmol) was stirred at 20° C. for 2 hours. The mixture was concentrated under reduced pressure to give 5-[rac-(2S,5R)-5-methylpiperazin-2-yl]-1,3-benzothiazole (3 g, crude, 2HCl) as a yellow solid.

### Step 8: Synthesis of (1-methylcyclopropyl)-[rac-(2R)-5-(1,3-benzothiazol-5-yl)-2-methyl-piperazin-1-yl]methanone

To a mixture of 5-[rac-(5R)-5-methylpiperazin-2-yl]-1,3-benzothiazole (1 g, 3.27 mmol, 2HCl) in DMF (10 mL) was added HATU (1.48 g, 3.89 mmol) and TEA (2.2 mL, 15.8 mmol). Then a solution of 1-methylcyclopropanecarboxylic acid (270 mg, 2.70 mmol) in DMF (3 mL) was added at −30° C. The resulting mixture was stirred at 0° C. for 2 hours. The mixture was purified by flash chromatography (Column: SepaFlash® Spherical C18, 60 g, 40-60 m, 120 Å; MeCN/water (0.05 v % NH₃—H₂O) with MeCN from 0-70%, 25 mL/min, 254 nm) to give (1-methylcyclopropyl)-[rac-(2R)-5-(1,3-benzothiazol-5-yl)-2-methyl-piperazin-1-yl]methanone (280 mg, 27.2% yield) as yellow solid. LCMS (ESI) [M+H]⁺ m/z: calcd 316.1, found 316.2.

### Step 9: Synthesis of 2-oxo-2-[rac-(2S,5R)-2-(1,3-benzothiazol-5-yl)-5-methyl-4-(1-methylcyclopropanecarbonyl)piperazin-1-yl]-N-[1-(2-trimethylsilylethoxymethyl)pyrazolo[4,3-c]pyridin-7-yl]acetamide

A mixture of (1-methylcyclopropyl)-[rac-(2R,5S)-5-(1,3-benzothiazol-5-yl)-2-methyl-piperazin-1-yl]methanone (250 mg, 0.793 mmol), 2-oxo-2-[[1-(2-trimethylsilylethoxymethyl)pyrazolo[4,3-c]pyridin-7-yl]amino]acetic acid (250 mg, 0.743 mmol), DMF (5 mL), HATU (350 mg, 0.921 mmol) and DIPEA (0.38 mL, 2.18 mmol) was stirred at 20° C. for 2 hours. The residue was purified by flash chromatography (Column: SepaFlash® Spherical C18, 15 g, 40-60 m, 120 Å; MeCN/water (0.05 v % NH₃—H₂O) with MeCN from 0-70%, 25 mL/min, 254 nm) to afford 2-oxo-2-[rac-(2S,5R)-2-(1,3-benzothiazol-5-yl)-5-methyl-4-(1-methylcyclopropanecarbonyl)piperazin-1-yl]-N-[1-(2-trimethylsilylethoxymethyl)pyrazolo[4,3-c]pyridin-7-yl]acetamide (180 mg, 35.8% yield) as yellow solid. LCMS (ESI) [M+H]Y m/z: calcd 634.3, found 634.3.

### Step 10: Synthesis of 2-oxo-N-(1H-pyrazolo[4,3-c]pyridin-7-yl)-2-[rac-(2S,5R)-2-(1,3-benzothiazol-5-yl)-5-methyl-4-(1-methylcyclopropanecarbonyl)piperazin-1-yl]acetamide

A mixture of 2-oxo-2-[rac-(2S,5R)-2-(1,3-benzothiazol-5-yl)-5-methyl-4-(1-methylcyclopropanecarbonyl)piperazin-1-yl]-N-[1-(2-trimethylsilylethoxymethyl)pyrazolo[4,3-c]pyridin-7-yl]acetamide (180 mg, 0.284 mmol) and TFA (5 mL, 64.9 mmol) was stirred at 20° C. for 2 hours. The mixture was concentrated under reduced pressure. The residue was purified by preparative HPLC (Instrument: Gilson GX-281 Liquid Handler, Gilson 322 Pump, Gilson 156 UV Detector; Column: Phenomenex Gemini-NX 80*40 mm*3 μm; Mobile phase A: H₂O with 10 mmol NH₄HCO3 (v %); Mobile phase B: MeCN; Gradient: B from 19% to 49% in 9.5 min, hold 100% B for 2 min; Flow Rate: 25 mL/min; Column Temperature: 30° C.; Wavelength: 220 nm, 254 nm) to afford 2-oxo-N-(1H-pyrazolo[4,3-c]pyridin-7-yl)-2-[rac-(2S,5R)-2-(1,3-benzothiazol-5-yl)-5-methyl-4-(1-methylcyclopropanecarbonyl)piperazin-1-yl]acetamide (50 mg, 35.0% yield) as white solid.

LCMS (ESI) [M+H]⁺ m/z: calcd 504.2, found 504.2.

### Step 11: Synthesis of 2-oxo-N-(1H-pyrazolo[4,3-c]pyridin-7-yl)-2-[rac-(2S,5R)-2-(1,3-benzothiazol-5-yl)-5-methyl-4-(1-methylcyclopropanecarbonyl)piperazin-1-yl]acetamide (Compound 61)

2-oxo-N-(1H-pyrazolo[4,3-c]pyridin-7-yl)-2-[rac-(5R)-2-(1,3-benzothiazol-5-yl)-5-methyl-4-(1-methylcyclopropanecarbonyl)piperazin-1-yl]acetamide (50 mg, 0.0993 mmol) was purified by SFC (Instrument: Berger, Multigr AM-II; Column: Daicel chiralpak AD (250 mm*30 mm*10 μm); Mobile phase: supercritical C₀₂/MeOH (0.1% NH₃—H₂O, IPA v %)=60/40; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm). The fraction was concentrated under reduced pressure and then lyophilized for overnight to give Compound 61.

Compound 61: 2-oxo-N-(1H-pyrazolo[4,3-c]pyridin-7-yl)-2-[rac-(2S,5R)-2-(1,3-benzothiazol-5-yl)-5-methyl-4-(1-methylcyclopropanecarbonyl)piperazin-1-yl]acetamide (49.5 mg, single known enantiomer with trans relative chemistry, Peak 2, Retention time: 6.389 min, white solid). ¹H NMR (400 MHz, methanol-d₄) δ ppm 9.12-9.41 (m, 1H), 8.99 (s, 1H), 8.49 (br s, 1H), 8.27-8.42 (m, 1H), 7.99-8.27 (m, 2H), 7.47-7.78 (m, 1H), 7.47-7.78 (m, 1H), 6.15 (br s, 1H), 5.21 (br d, J=13.1 Hz, 1H), 4.71 (br s, 1H), 4.35 (br d, J=11.5 Hz, 1H), 3.44-3.70 (m, 1H), 1.27-1.69 (m, 4H), 0.93-1.24 (m, 4H), 0.49-0.83 (m, 3H); LCMS (ESI) [M+H]⁺ m/z: calcd 504.2, found 504.2; HPLC: 99.67%®220 nm, 99.48%®254 nm; 99.9% ee.

### Example 8. Compound 17 N-(4-amino-1H-pyrazolo[4,3-c]pyridin-7-yl)-2-oxo-2-[rac-(2S,5R)-2-(1,3-benzothiazol-5-yl)-5-methyl-4-[1-(trifluoromethyl)cyclopropanecarbonyl]piperazin-1-yl]acetamide

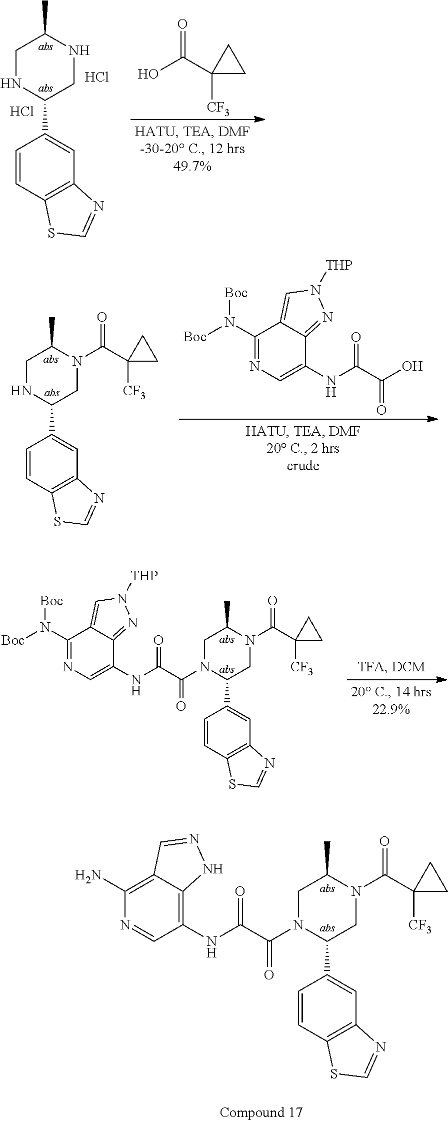

### Step 1: Synthesis of [rac-(2R,5S)-5-(1,3-benzothiazol-5-yl)-2-methyl-piperazin-1-yl]-[1-(trifluoromethyl)cyclopropyl]methanone

To a mixture of 5-[rac-(2S,5R)-5-methylpiperazin-2-yl]-1,3-benzothiazole (100 mg, 0.327 mmol, 2HCl) in DMF (3 mL) were added HATU (148 mg, 0.389 mmol) and TEA (1.61 mmol, 0.225 mL). Then 1-(trifluoromethyl)cyclopropanecarboxylic acid (55 mg, 0.357 mmol) in (DMF(2 mL)) was added at −30° C. The resulting mixture was stirred at 20° C. for 12 hours. The mixture product was purified by flash chromatography (Column: SepaFlash® Spherical C₁₈, 40 g, 40-60 m, 120 Å; MeCN/water (0.05 v % NH₃—H₂O) with MeCN from 0-30%, 40 mL/min, 254 nm) to afford [rac-(2R,5S)-5-(1,3-benzothiazol-5-yl)-2-methyl-piperazin-1-yl]-[1-(trifluoromethyl)cyclopropyl]methanone (60 mg, 49.7% yield) as white solid.

LCMS (ESI) [M+H]⁺ m/z: calcd 370.1, found 370.2.

### Step 2: Synthesis of tert-butyl N-tert-butoxycarbonyl-N-[7-[[2-oxo-2-[rac-(2S,5R)-2-(1,3-benzothiazol-5-yl)-5-methyl-4-[1-(trifluoromethyl)cyclopropanecarbonyl]piperazin-1-yl]acetyl]amino]-2-tetrahydropyran-2-yl-pyrazolo[4,3-c]pyridin-4-yl]carbamate

A mixture of [rac-(2R,5S)-5-(1,3-benzothiazol-5-yl)-2-methyl-piperazin-1-yl]-[1-(trifluoromethyl)cyclopropyl]methanone (50 mg, 0.135 mmol), 2-[[4-[bis(tert-butoxycarbonyl)amino]-2-tetrahydropyran-2-yl-pyrazolo[4,3-c]pyridin-7-yl]amino]-2-oxo-acetic acid (60 mg, 0.119 mmol), HATU (57 mg, 0.150 mmol), TEA (0.1 mL, 0.717 mmol) in DMF (3 mL) was stirred at 20° C. for 2 hours. The resulting mixture was quenched by addition of water (50 mL) and extracted with EtOAc (100 mL*3). The combined organic layer was washed with brine (100 mL), dried over anhydrous Na₂SO₄, filtered and concentrated under reduced pressure. The residue was purified by preparative TLC (silica, DCM/MeOH=15:1, 254 nm) to afford tert-butyl N-tert-butoxycarbonyl-N-[7-[[2-oxo-2-[rac-(2S,5R)-2-(1,3-benzothiazol-5-yl)-5-methyl-4-[1-(trifluoromethyl)cyclopropanecarbonyl]piperazin-1-yl]acetyl]amino]-2-tetrahydropyran-2-yl-pyrazolo[4,3-c]pyridin-4-yl]carbamate (80 mg, crude) as a yellow oil.

### Step 3: Synthesis of N-(4-amino-1H-pyrazolo[4,3-c]pyridin-7-yl)-2-oxo-2-[rac-(2S,5R)-2-(1,3-benzothiazol-5-yl)-5-methyl-4-[1-(trifluoromethyl)cyclopropanecarbonyl]piperazin-1-yl]acetamide(Compound 17)

A mixture of tert-butyl N-tert-butoxycarbonyl-N-[7-[[2-oxo-2-[rac-(2S,5R)-2-(1,3-benzothiazol-5-yl)-5-methyl-4-[1-(trifluoromethyl)cyclopropanecarbonyl]piperazin-1-yl]acetyl]amino]-2-tetrahydropyran-2-yl-pyrazolo[4,3-c]pyridin-4-yl]carbamate (70 mg, 81.7 mol), DCM (1 mL) and TFA (0.4 mL, 5.20 mmol) was stirred at 20° C. for 14 hours. The residue was purified by preparative TLC (silica, DCM/MeOH=1:1, 254 nm) to afford product (40 mg, 64.7% purify) as a yellow solid. The mixture was adjusted pH=8 with NH₃—H₂O (12 N), Then the mixture was purified by flash chromatography (Column: SepaFlash® Spherical C18, 20 g, 40-60 m, 120 Å; MeCN/water (0.05 v % NH₃—H₂O) with MeCN from 0-38%, 30 mL/min, 254 nm) to give N-(4-amino-1H-pyrazolo[4,3-c]pyridin-7-yl)-2-oxo-2-[rac-(2S,5R)-2-(1,3-benzothiazol-5-yl)-5-methyl-4-[1-(trifluoromethyl)cyclopropanecarbonyl]piperazin-1-yl]acetamide (10.7 mg, 22.9% yield) as white solid. ¹H NMR (400 MHz, methanol-d₄) δ ppm 9.19-9.30 (m, 1H), 8.26 (br s, 1H), 8.00-8.18 (m, 2H), 7.75 (br s, 1H), 7.46-7.64 (m, 1H), 6.17 (br s, 1H), 5.26 (br d, J=14.3 Hz, 1H), 4.16-4.39 (m, 2H), 3.50-3.70 (m, 1H), 1.19-1.55 (m, 7H), 1.05 (br s, 1H); ¹⁹F NMR (376 MHz, methanol-d₄)₆ ppm -68.81; LCMS (ESI) [M+H]⁺ m/z: calcd 573.2, found 573.3; HPLC: 99.16% at 254 nm; SFC: 90.6% ee.

### Example 9. Compound 76 N-(4-amino-2H-pyrazolo[4,3-c]pyridin-7-yl)-2-oxo-2-[rac-(2S,5R)-4-acetyl-2-(1,3-benzothiazol-5-yl)-5-methyl-piperazin-1-yl]acetamide

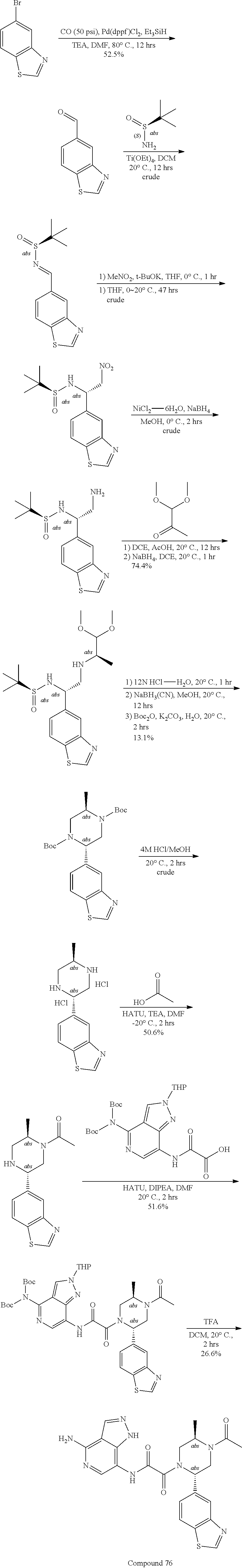

### Step 1: Synthesis of 1,3-benzothiazole-5-carbaldehyde

A mixture of 5-bromo-1,3-benzothiazole (25 g, 117 mmol), Pd(dppf)C₁₂ (4.37 g, 5.98 mmol), TEA (55.0 mL, 395 mmol), TES (43.7 g, 376 mmol) and DMF (100 mL) was stirred at 80° C. for 20 hours under CO (50 Psi). The resulting mixture was quenched by addition of water (300 mL) and extracted with EtOAc (200 mL*3). The combined organic layer was washed with saturated NH₄Cl aqueous solution (100 mL), brine (100 mL), dried over anhydrous Na₂SO₄, filtered and concentrated under reduced pressure to give residue which was purified by flash chromatography (ISCO®; 120 g AgelaFlash® Silica Flash Column, petroleum ether/EtOAc with EtOAc from 0˜50%, Flow Rate:30 mL/min, 254 nm) to afford 1,3-benzothiazole-5-carbaldehyde (10 g, 52.5% yield) as yellow solid.

LCMS (ESI) [M+H]⁺ m/z: calcd 164.0, found 163.8

### Step 2: Synthesis of rac-(NE,S)—N-(1,3-benzothiazol-5-ylmethylene)-2-methyl-propane-2-sulfinamide

To a solution of 1,3-benzothiazole-5-carbaldehyde (10 g, 61.3 mmol), rac-(S)-2-methylpropane-2-sulfinamide (10 g, 82.5 mmol) in THF (100 mL) was added Ti(OEt)₄ (21 g, 92.1 mmol). The mixture was stirred at 60° C. for 12 hours. The resulting mixture was quenched by addition of brine (20 mL), filtered and the filter cake washed with ethyl acetate (200 mL*2). The filtrate was washed with brine (200 mL), dried over Na₂SO₄, filtered and concentrated under reduced pressure to afford rac-(NE,S)—N-(1,3-benzothiazol-5-ylmethylene)-2-methyl-propane-2-sulfinamide (16 g, crude) as yellow solid.

¹H NMR (400 MHz, methanol-d₄) δ ppm 9.39 (s, 1H), 8.76 (s, 1H), 8.58 (br s, 1H), 8.19-8.32 (m, 1H), 8.09 (br d, J=8.1 Hz, 1H), 1.32 (s, 10H). LCMS (ESI) [M+H]⁺ m/z: calcd 267.1, found 266.9

### Step 3: Synthesis of N-[(1R)-1-(1,3-benzothiazol-5-yl)-2-nitro-ethyl]-2-methyl-propane-2-sulfinamide

A mixture of potassium;2-methylpropan-2-olate (1 M in THF, 300 mL) and THF (170 mL) was cooled to 0° C. then nitromethane (61 g, 999 mmol) was added. The mixture was stirred at 0° C. for 1 hour. The (NZ)—N-(1,3-benzothiazol-5-ylmethylene)-2-methyl-propane-2-sulfinamide (15 g, 56.3 mmol) was added to the mixture at 0° C. and then stirred at 20° C. for 12 hours. The resulting mixture was concentrated under reduced pressure to give a residue which was purified by flash chromatography (ISCO®; 40 g AgelaFlash® Silica Flash Column, petroleum ether/EtOAc with EtOAc from 0˜60%, Flow Rate:50 mL/min, 254 nm) to afford N-[(1R)-1-(1,3-benzothiazol-5-yl)-2-nitro-ethyl]-2-methyl-propane-2-sulfinamide (13 g, crude) as yellow solid.

¹H NMR (400 MHz, methanol-d₄) δ ppm 9.31 (s, 1H), 8.08-8.21 (m, 2H), 7.58 (dd, J 8.4, 1.6 Hz, 1H), 5.31-5.40 (m, 1H), 5.06-5.15 (m, 1H), 4.98 (dd, J 13.4, 6.3 Hz, 1H), 1.21 (s, 9H). LCMS (ESI) [M+H]⁺ m/z: calcd 328.1, found 328.1

### Step 4: Synthesis of N-[(1R)-2-amino-1-(1,3-benzothiazol-5-yl)ethyl]-2-methyl-propane-2-sulfinamide

A mixture of N-[(1R)-1-(1,3-benzothiazol-5-yl)-2-nitro-ethyl]-2-methyl-propane-2-sulfinamide (8 g, 24.4 mmol), dichloronickel;hexahydrate (6.40 g, 26.9 mmol) and MeOH (100 mL) was cooled to 0° C. then the NaBH4 (5.42 g, 143 mmol) was added slowly and was stirred at 0° C. for 2 hours. The mixture was filtered and concentrated (30° C.) under reduced pressure to give N-[(1R)-2-amino-1-(1,3-benzothiazol-5-yl)ethyl]-2-methyl-propane-2-sulfinamide (10 g, crude) as black oil.

LCMS (ESI) [M+H]⁺ m/z: calcd 298.1, found 298.0

### Step 5: Synthesis of N-[(1S)-1-(1,3-benzothiazol-5-yl)-2-[[(1R)-2,2-dimethoxy-1-methyl-ethyl]amino]ethyl]-2-methyl-propane-2-sulfinamide

A mixture of N-[(1S)-2-amino-1-(1,3-benzothiazol-5-yl)ethyl]-2-methyl-propane-2-sulfinamide (7 g, 23.5 mmol), 1,1-dimethoxypropan-2-one (7.47 g, 63.2 mmol), Na₂SO₄ (12 g, 84.8 mmol), AcOH (1.50 g, 25.0 mmol) and DCE (100 mL) was stirred at 20° C. for 12 hours. The NaBH4 (2.9 g, 75.5 mmol) was added to the mixture and then stirred at 20° C. for 1 hour. The resulting mixture was quenched by addition of water (50 mL) and extracted with DCM (100 mL*3). The combined organic layer was washed with saturated NH₄Cl aqueous solution (50 mL*2), brine (50 mL), dried over anhydrous Na₂SO₄, filtered and concentrated under reduced pressure to give N-[(1S)-1-(1,3-benzothiazol-5-yl)-2-[[(1R)-2,2-dimethoxy-1-methyl-ethyl]amino]ethyl]-2-methyl-propane-2-sulfinamide (7 g, 74.4% yield) as yellow oil.

LCMS (ESI) [M+H]⁺ m/z: calcd 400.2, found 400.1

### Step 6: Synthesis of ditert-butyl (2S,5R)-2-(1,3-benzothiazol-5-yl)-5-methyl-piperazine-1,4-dicarboxylate

A mixture of N-[(1S)-1-(1,3-benzothiazol-5-yl)-2-[[(1R)-2,2-dimethoxy-1-methyl-ethyl]amino]ethyl]-2-methyl-propane-2-sulfinamide (4.5 g, 11.3 mmol) and HCl/H₂O (12 M, 12 mL) was stirred at 20° C. for 1 hours. Then the mixture was concentrated under reduced pressure to give a residue which was diluted with MeOH (30 mL). NaBH3(CN) (700 mg, 11.1 mmol), Na₂SO₄ (5.4 g, 38.0 mmol) and MeOH (30 mL) was added and the mixture was stirred at 20° C. for 12 hours. The mixture was added Boc₂O (8.56 g, 39.2 mmol), K₂CO₃ (4.8 g, 34.7 mmol), H₂O (15 mL) and the mixture was stirred at 20° C. for 2 hrs. The resulting mixture was quenched by addition of water (100 mL) and extracted with EtOAc (100 mL*3). The combined organic layer was washed with brine (100 mL), dried over anhydrous Na₂SO₄, filtered and concentrated under reduced pressure to give a residue which was purified by flash chromatography (ISCO®; 12 g AgelaFlash® Silica Flash Column, petroleum ether/EtOAc with EtOAc from 0˜30%, Flow Rate: 30 mL/min, 254 nm) to afford ditert-butyl (2S,5R)-2-(1,3-benzothiazol-5-yl)-5-methyl-piperazine-1,4-dicarboxylate (0.65 g, 13.3% yield) as yellow oil.

LCMS (ESI) [M+H]⁺ m/z: calcd 434.2, found 234.3

### Step 7: Synthesis of 5-[rac-(2S,5R)-5-methylpiperazin-2-yl]-1,3-benzothiazole

A mixture of ditert-butyl rac-(2S,5R)-2-(1,3-benzothiazol-5-yl)-5-methyl-piperazine-1,4-dicarboxylate (1.00 g, 2.31 mmol) and MeOH/HCl (4 M, 5 mL) was stirred at 20° C. for 2 hours. The mixture was concentrated under reduced pressure to give 5-[rac-(2S,5R)-5-methylpiperazin-2-yl]-1,3-benzothiazole (560 mg, crude, 2HCl) as yellow solid.

LCMS (ESI) [M+H]⁺ m/z: calcd 234.1, found 234.2

### Step 8: Synthesis of 1-[rac-(2R,5S)-5-(1,3-benzothiazol-5-yl)-2-methyl-piperazin-1-yl]ethanone

To a solution of acetic acid (20 mg, 0.333 mmol) in DMF (3 mL) was added HATU (205 mg, 0.540 mmol) and TEA (110 μL, 0.798 mmol). The mixture was stirred at 20° C. for 20 minutes. Then the resulting mixture was added to a solution of 5-[rac-(2S,5R)-5-methylpiperazin-2-yl]-1,3-benzothiazole (110 mg, 0.359 mmol, 2HCl), TEA (125 μL, 0.898 mmol) in at −20° C. The resulting mixture was stirred at −20° C. for 2 hours. The resulting mixture was quenched by addition of water (10 mL) and extracted with DCM (20 mL*3). The combined organic layers were washed with brine (100 mL), dried over anhydrous Na₂SO₄, filtered and concentrated under reduced pressure. The residue was purified by flash chromatography (ISCO®; 12 g AgelaFlash® Silica Flash Column, DCM/MeOH with MeOH from 0˜10%, Flow Rate: 30 mL/min, 254 nm) to afford 1-[rac-(2R,5S)-5-(1,3-benzothiazol-5-yl)-2-methyl-piperazin-1-yl]ethanone (50 mg, 50.6% yield) as light-yellow solid.

LCMS (ESI) [M+H]⁺ m/z: calcd 276.1, found 276.2

### Step 9: Synthesis of tert-butyl N-tert-butoxycarbonyl-N-[7-[[2-oxo-2-[rac-(2S,5R)-4-acetyl-2-(1,3-benzothiazol-5-yl)-5-methyl-piperazin-1-yl]acetyl]amino]-2-tetrahydropyran-2-yl-pyrazolo[4,3-c]pyridin-4-yl]carbamate

To a solution of 1-[rac-(2R,5S)-5-(1,3-benzothiazol-5-yl)-2-methyl-piperazin-1-yl]ethanone (21 mg, 76.3 μmol), 2-[[4-[bis(tert-butoxycarbonyl)amino]-2-tetrahydropyran-2-yl-pyrazolo[4,3-c]pyridin-7-yl]amino]-2-oxo-acetic acid (42 mg, 83.1 mol), HATU (47 mg, 0.124 mmol) in DMF (3 mL) was added DIPEA (42.0 μL, 0.241 mmol). The mixture was stirred at 20° C. for 1 hour. The resulting mixture was quenched by addition of water (10 mL) and extracted with DCM (20 mL*3). The combined organic layers were washed with brine (30 mL), dried over anhydrous Na₂SO₄, filtered and concentrated under reduced pressure. The residue was purified by flash chromatography (Biotage®, Column: SepaFlash® Spherical C18, 40 g, 40-60 μm, 120 Å; MeCN/water (0.05% NH₃—H₂O) with MeCN from 0˜30%, 30 mL/min, 254 nm) to afford crude product (100 mg). The crude product was purified by prepare-TLC (DCM/MeOH=20/1, 254 nm) to afford tert-butyl N-tert-butoxycarbonyl-N-[7-[[2-oxo-2-[rac-(2S,5R)-4-acetyl-2-(1,3-benzothiazol-5-yl)-5-methyl-piperazin-1-yl]acetyl]amino]-2-tetrahydropyran-2-yl-pyrazolo[4,3-c]pyridin-4-yl]carbamate (30 mg, 51.6% yield) as yellow solid.

LCMS (ESI) [M+H]⁺ m/z: calcd 763.3, found 763.5.

### Step 10: Synthesis of N-(4-amino-2H-pyrazolo[4,3-c]pyridin-7-yl)-2-oxo-2-[rac-(2S,5R)-4-acetyl-2-(1,3-benzothiazol-5-yl)-5-methyl-piperazin-1-yl]acetamide (Compound 76)

To a solution of tert-butyl N-tert-butoxycarbonyl-N-[7-[[2-oxo-2-[rac-(2S,5R)-4-acetyl-2-(1,3-benzothiazol-5-yl)-5-methyl-piperazin-1-yl]acetyl]amino]-2-tetrahydropyran-2-yl-pyrazolo[4,3-c]pyridin-4-yl]carbamate (30.00 mg, 39.3 mol) in DCM (5 mL) was added TFA (1 mL). The mixture was stirred at 20° C. for 12 hours. The resulting mixture was concentrated under reduced pressure, the residue was diluted with MeOH, and adjusted to pH=8 with NH₃—H₂O. The mixture was purified by flash chromatography(Biotage®, Column: SepaFlash® Spherical C18, 40 g, 40-60 μm, 120 Å; MeCN/water (0.05% NH₃—H₂O) with MeCN from 0˜20%, 30 mL/min, 254 nm) to afford N-(4-amino-2H-pyrazolo[4,3-c]pyridin-7-yl)-2-oxo-2-[rac-(2S,5R)-4-acetyl-2-(1,3-benzothiazol-5-yl)-5-methyl-piperazin-1-yl]acetamide (5.0 mg, 26.6% yield) as white solid.

¹H NMR (400 MHz, methanol-d₄) δ ppm 9.25-9.33 (m, 1H), 8.21-8.29 (m, 1H), 8.07-8.19 (m, 2H), 7.54-7.76 (m, 2H), 6.10 (br s, 1H), 5.22 (br d, J=14.6 Hz, 1H), 4.04-4.36 (m, 3H), 3.57-3.65 (m, 1H), 3.46-3.53 (m, 1H), 3.01 (s, 1H), 2.88 (s, 1H), 2.13-2.26 (m, 3H), 1.45 (dd, J=11.3, 6.8 Hz, 2H), 1.34 (d, J=6.5 Hz, 2H). LCMS (ESI) [M+H]⁺ m/z: calcd 479.2, found 479.3; HPLC: 88.73% at 254 nm; SFC: 100% ee.

### Example 10. Compound 70 N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide

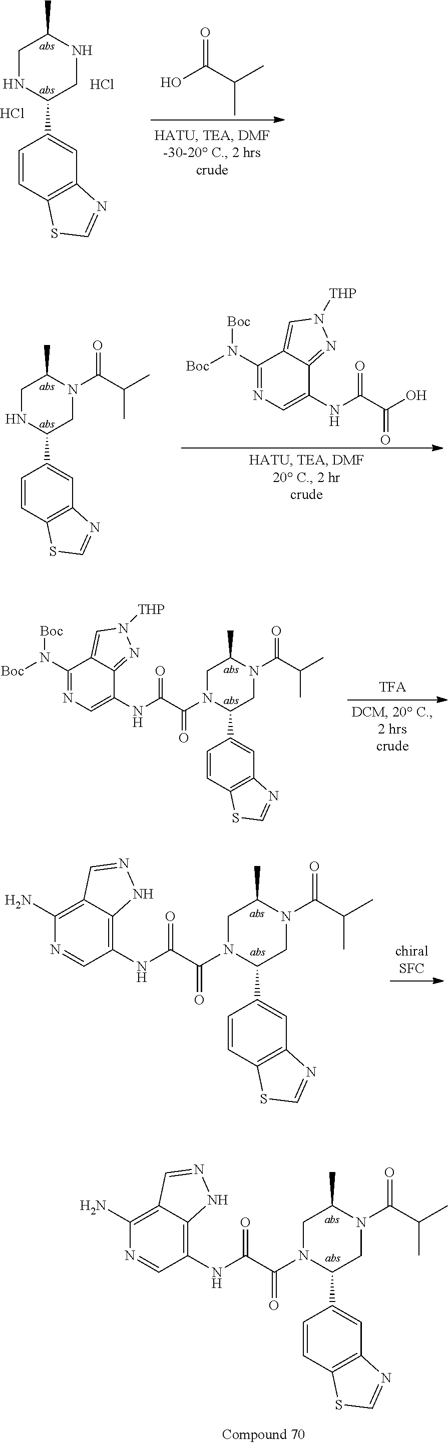

### Step 1: Synthesis of 2-methyl-1-[rac-(2R,5S)-5-(1,3-benzothiazol-5-yl)-2-methyl-piperazin-1-yl]propan-1-one

To a mixture of 5-[rac-(2S,5R)-5-methylpiperazin-2-yl]-1,3-benzothiazole (100 mg, 0.327 mmol, 2HCl) in DMF(3 mL) were added HATU (140 mg, 0.368 mmol) and TEA (0.200 mL, 1.43 mmol). Then 2-methylpropanoic acid (30 mg, 0.341 mmol) in DMF (2 mL)) was added dropwise at −30° C. The resulting mixture was stirred at 20° C. for 2 hours. The mixture was purified by flash chromatography (Column: SepaFlash® Spherical C18, 15 g, 40-60 m, 120 Å; MeCN/water (0.05 v % NH₃—H₂O) with MeCN from 0˜30%, 25 mL/min, 254 nm). The fraction was concentrated under reduced pressure and then lyophilized for overnight to afford 2-methyl-1-[rac-(2R,5S)-5-(1,3-benzothiazol-5-yl)-2-methyl-piperazin-1-yl]propan-1-one (70 mg, crude) as white solid. LCMS (ESI) [M+H]⁺ m/z: calcd 304.1, found 304.2.

### Step 2: Synthesis of tert-butyl N-tert-butoxycarbonyl-N-[7-[[2-oxo-2-[rac-(2S,5R)-2-(1,3-benzothiazol-5-yl)-5-methyl-4-(2-methylpropanoyl)piperazin-1-yl]acetyl]amino]-2-tetrahydropyran-2-yl-pyrazolo[4,3-c]pyridin-4-yl]carbamate

A mixture of 2-methyl-1-[rac-(2R,5S)-5-(1,3-benzothiazol-5-yl)-2-methyl-piperazin-1-yl]propan-1-one (50.0 mg, 0.115 mmol), 2-[[4-[bis(tert-butoxycarbonyl)amino]-2-tetrahydropyran-2-yl-pyrazolo[4,3-c]pyridin-7-yl]amino]-2-oxo-acetic acid (60.0 mg, 0.119 mmol), HATU (52 mg, 0.137 mmol), DMF (3 mL) and TEA (80 μL, 0.574 mmol) was stirred at 20° C. for 2 hours. The resulting mixture was concentrated under reduced pressure. The residue was purified by flash chromatography (ISCO®; 4 g AgelaFlash® Silica Flash Column, petroleum ether/EtOAc with EtOAc from 0˜90%, flow rate=30 mL/min, 254 nm) to afford tert-butyl N-tert-butoxycarbonyl-N-[7-[[2-oxo-2-[rac-(2S,5R)-2-(1,3-benzothiazol-5-yl)-5-methyl-4-(2-methylpropanoyl)piperazin-1-yl]acetyl]amino]-2-tetrahydropyran-2-yl-pyrazolo[4,3-c]pyridin-4-yl]carbamate (110 mg, crude) as yellow solid. LCMS (ESI) [M+H]⁺ m/z: calcd 791.4, found 791.5.

### Step 3: Synthesis of N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide

A mixture of tert-butyl N-tert-butoxycarbonyl-N-[7-[[2-oxo-2-[rac-(2S,5R)-2-(1,3-benzothiazol-5-yl)-5-methyl-4-(2-methylpropanoyl)piperazin-1-yl]acetyl]amino]-2-tetrahydropyran-2-yl-pyrazolo[4,3-c]pyridin-4-yl]carbamate (100 mg, 0.126 mmol), DCM (4 mL) and TFA (0.2 mL, 2.60 mmol) was stirred at 20° C. for 2 hours. The resulting mixture was adjusted to pH=8 with NH₃—H₂O.

The mixture was concentrated under reduced pressure to give a crude product, which was purified by preparative HPLC (Instrument: Gilson GX-281 Liquid Handler, Gilson 322 Pump, Gilson 156 UV Detector; Column: 2—Phenomenex Gemini C₁₈ 75*40 mm*3 m; Mobile phase A: H₂O with 0.05% NH₃—H₂O (v %); Mobile phase B: MeCN; Gradient: B from 12% to 42% in 9.5 min, hold 100% B for 3 min; Flow Rate: 25 mL/min; Column Temperature: 30° C.; Wavelength: 220 nm, 254 nm) to afford N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide (10 mg, crude) as a white solid. LCMS (ESI) [M+H]⁺ m/z: calcd 507.2, found 507.3.

### Step 4: Synthesis of N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide(Compound 70)

N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide (10.0 mg, 19.7 μmol) was purified by SFC (Instrument: Berger, MULTIGR AM-II; Column: DAICEL CHIRALCEL OJ (250 mm*30 mm*10 μm); Mobile phase: supercritical Hexane-IPA (0.1% NH₃, MeOH v %)=70/30; Flow Rate:70 mL/min; Column Temperature: 38° C.; Nozzle Pressure: 100 bar; Nozzle Temperature: 60° C.; Evaporator Temperature: 20° C.; Trimmer Temperature: 25° C.; Wavelength:220 nm). The fraction was concentrated under reduced pressure and then lyophilized for overnight.

Compound 70: N-(4-amino-1H-pyrazolo[4,3-c]pyridin-7-yl)-2-oxo-2-[rac-(2S,5R)-2-(1,3-benzothiazol-5-yl)-5-methyl-4-(2-methylpropanoyl)piperazin-1-yl]acetamide (4.9 mg, single known enantiomer with trans relative chemistry, white solid). ¹H NMR (400 MHz, methanol-d₄) δ ppm 9.15-9.35 (m, 1H), 8.16-8.26 (m, 1H), 8.03-8.14 (m, 2H), 7.47-7.81 (m, 2H), 5.68-6.12 (m, 1H), 5.21 (br d, J=14.5 Hz, 1H), 4.28-4.42 (m, 1H), 3.90-4.17 (m, 1H), 3.41-3.64 (m, 1H), 2.56-3.13 (m, 1H), 1.44 (dd, J=10.5, 6.8 Hz, 2H), 1.27-1.35 (m, 2H), 1.13 (dd, J=6.6, 1.5 Hz, 2H), 0.96-1.05 (m, 3H), 0.69 (dd, J=8.9, 6.6 Hz, 1H); LCMS (ESI) [M+H]⁺ m/z: calcd 507.2, found 507.3; HPLC: 100% at 254 nm; SFC: 93.6% ee.

### Example 11. Compound 39 N-(4-amino-1H-pyrazolo[4,3-c]pyridin-7-yl)-2-oxo-2-[rac-(2S,5R)-2-(1,3-benzothiazol-5-yl)-4-(2,2-dimethylpropanoyl)-5-methyl-piperazin-1-yl]acetamide

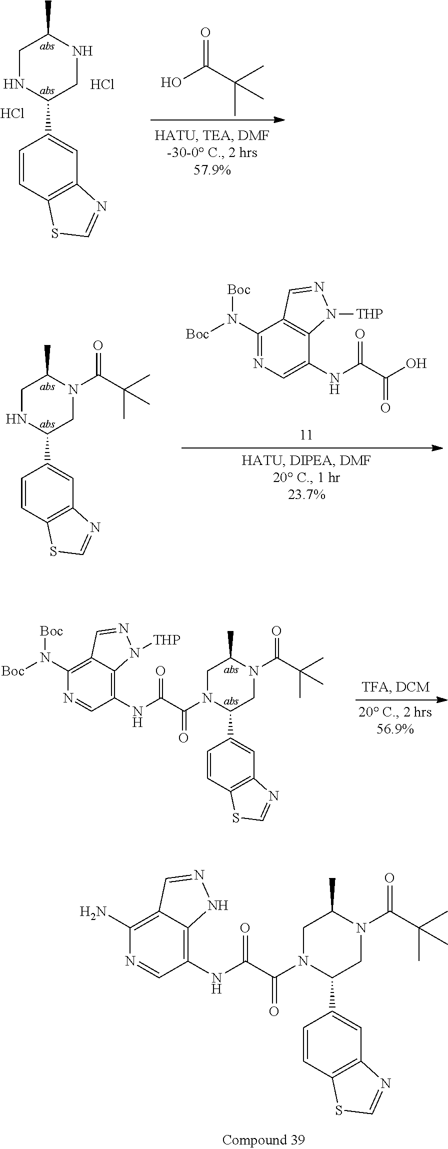

### Step 1: Synthesis of 2,2-dimethyl-1-[rac-(2R,5S)-5-(1,3-benzothiazol-5-yl)-2-methyl-piperazin-1-yl]propan-1-one

To a mixture of 5-[rac-(2S,5R)-5-methylpiperazin-2-yl]-1,3-benzothiazole (100 mg, 0.326 mmol, 2HCl) in DMF (6 mL) was added HATU (188 mg, 0.494 mol) and TEA (200 μL, 1.43 mmol). Then a solution of 2,2-dimethylpropanoic acid (37 mg, 0.362 mmol) in DMF (3 mL) was added at −30° C. The resulting mixture was stirred at 0° C. for 2 hours. The resulting mixture was concentrated under reduced pressure. The residue was purified by flash chromatography (Biotage®, Column: SepaFlash® Spherical C18, 40 g, 40-60 m, 120 Å; MeCN/water (0.05% NH₃—H₂O) with MeCN from 0˜32%, 30 mL/min, 254 nm) to afford 2,2-dimethyl-1-[rac-(2R,5S)-5-(1,3-benzothiazol-5-yl)-2-methyl-piperazin-1-yl]propan-1-one (60 mg, 57.9% yield) as light-yellow solid.

LCMS (ESI) [M+H]⁺ m/z: calcd 318.2, found 318.2.

### Step 2: Synthesis of tert-butyl N-tert-butoxycarbonyl-N-[7-[[2-oxo-2-[rac-(2S,5R)-2-(1,3-benzothiazol-5-yl)-4-(2,2-dimethylpropanoyl)-5-methyl-piperazin-1-yl]acetyl]amino]-2-tetrahydropyran-2-yl-pyrazolo[4,3-c]pyridin-4-yl]carbamate

To a solution of 2,2-dimethyl-1-[rac-(2R,5S)-5-(1,3-benzothiazol-5-yl)-2-methyl-piperazin-1-yl]propan-1-one (50 mg, 157 mol), 2-[[4-[bis(tert-butoxycarbonyl)amino]-2-tetrahydropyran-2-yl-pyrazolo[4,3-c]pyridin-7-yl]amino]-2-oxo-acetic acid (65 mg, 129 mol), HATU (80 mg, 210 mol) in DMF (5 mL) was added DIPEA (50 μL, 287 mol). The mixture was stirred at 20° C. for 1 hour. The resulting mixture was concentrated under reduced pressure. The residue was purified by flash chromatography (Biotage®, Column: SepaFlash® Spherical C18, 40 g, 40-60 m, 120 Å; MeCN/water (0.05% NH₃—H₂O) with MeCN from 0˜32%, 30 mL/min, 254 nm) to afford tert-butyl N-tert-butoxycarbonyl-N-[7-[[2-oxo-2-[rac-(2S,5R)-2-(1,3-benzothiazol-5-yl)-4-(2,2-dimethylpropanoyl)-5-methyl-piperazin-1-yl]acetyl]amino]-2-tetrahydropyran-2-yl-pyrazolo[4,3-c]pyridin-4-yl]carbamate (30 mg, 23.7% yield) as a yellow solid. LCMS (ESI) [M+H]⁺ m/z: calcd805.4, found 805.5.

### Step 3: Synthesis of N-(4-amino-1H-pyrazolo[4,3-c]pyridin-7-yl)-2-oxo-2-[rac-(2S,5R)-2-(1,3-benzothiazol-5-yl)-4-(2,2-dimethylpropanoyl)-5-methyl-piperazin-1-yl]acetamide (Compound 39)

To a solution of tert-butyl N-tert-butoxycarbonyl-N-[7-[[2-oxo-2-[rac-(2S,5R)-2-(1,3-benzothiazol-5-yl)-4-(2,2-dimethylpropanoyl)-5-methyl-piperazin-1-yl]acetyl]amino]-2-tetrahydropyran-2-yl-pyrazolo[4,3-c]pyridin-4-yl]carbamate (25 mg, 31.1 mol) in DCM (5 mL) was added TFA (0.5 mL). The mixture was stirred at 20° C. for 1 hours. The resulting mixture was concentrated under reduced pressure. The residue was purified by flash chromatography (Biotage®, Column: SepaFlash® Spherical C18, 40 g, 40-60 μm, 120 Å; MeCN/water (0.05% NH₃—H₂O) with MeCN from 0˜25%, 30 mL/min, 254 nm) to afford N-(4-amino-1H-pyrazolo[4,3-c]pyridin-7-yl)-2-oxo-2-[rac-(2S,5R)-2-(1,3-benzothiazol-5-yl)-4-(2,2-dimethylpropanoyl)-5-methyl-piperazin-1-yl]acetamide (9.2 mg, 56.9% yield) as white solid.

¹H NMR (400 MHz, methanol-d₄) δ ppm 9.27 (d, J=10.0 Hz, 1H), 8.19-8.28 (m, 1H), 8.03-8.18 (m, 2H), 7.72-7.83 (m, 1H), 7.55-7.66 (m, 1H), 6.03 (br s, 1H), 5.36 (t, J=4.6 Hz, 1H), 4.25 (br d, J=14.3 Hz, 1H), 3.50-3.85 (m, 1H), 2.21 (t, J=7.7 Hz, 1H), 2.01-2.09 (m, 1H), 1.62 (br s, 1H), 1.41-1.52 (m, 3H), 1.33-1.40 (m, 4H), 1.11-1.25 (m, 11H). LCMS (ESI) [M+H]Y m/z: calcd 521.2, found 521.4. HPLC: 94.85%®254 nm; SFC: 90.2% ee.

### Example 12. Compound 43 and Compound 45 N-(6-amino-5-methyl-3-pyridyl)-2-[(2S,5R)-2-(1,3-benzothiazol-5-yl)-5-methyl-4-(2-methylpropanoyl)piperazin-1-yl]-2-oxo-acetamide

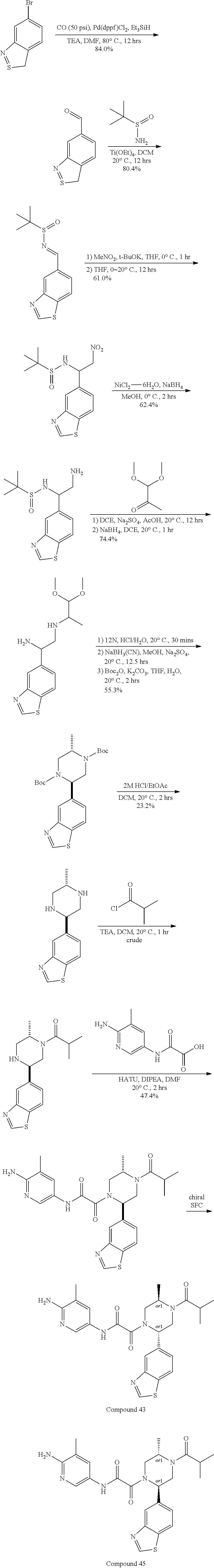

### Step 1: Synthesis of 1,3-benzothiazole-5-carbaldehyde

A mixture of 5-bromo-1,3-benzothiazole (5 g, 23.4 mmol), Pd(dppf)C₁₂ (875 mg, 1.20 mmol), TEA (7.78 g, 76.9 mmol), TES (8.57 g, 73.7 mmol) and DMF (40 mL) was stirred at 80° C. for 12 hours under CO (50 psi). The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 40 g AgelaFlash® Silica Flash Column, petroleum ether/EtOAc with EtOAc from 0-42%, Flow Rate: 30 mL/min, 254 nm) to afford 1,3-benzothiazole-5-carbaldehyde (3.2 g, 84.0% yield) as white solid. LCMS (ESI) [M+H]⁺ m/z: calcd 164.0, found 164.0.

### Step 2: Synthesis of (NZ)—N-(1,3-benzothiazol-5-ylmethylene)-2-methyl-propane-2-sulfinamide

A mixture of 1,3-benzothiazole-5-carbaldehyde (3.2 g, 19.6 mmol), 2-methylpropane-2-sulfinamide (2.85 g, 23.5 mmol), Ti(OEt)₄ (14 g, 61.4 mmol) and DCM (50 mL) was stirred at 20° C. for 12 hours. The resulting mixture was quenched by addition of water (100 mL) and extracted with DCM (100 mL*3). The combined organic layer was washed with saturated NH₄Cl aqueous solution (100 mL*2), brine (100 mL), dried over anhydrous Na₂SO₄, filtered and concentrated under reduced pressure to give (NZ)—N-(1,3-benzothiazol-5-ylmethylene)-2-methyl-propane-2-sulfinamide (4.2 g, 80.4% yield) as yellow oil.

LCMS (ESI) [M+H]⁺ m/z: calcd 267.0, found 267.0.

### Step 3: Synthesis of N—[1-(1,3-benzothiazol-5-yl)-2-nitro-ethyl]-2-methyl-propane-2-sulfinamide

A mixture of 1M t-BuOK/THF (67 mL, 67.0 mmol) and THF (50 mL) was cooled to 0° C. then nitromethane (14 mL, 0.259 mol) was added slowly. The mixture was stirred at 0° C. for 1 hour. The (NZ)—N-(1,3-benzothiazol-5-ylmethylene)-2-methyl-propane-2-sulfinamide (4 g, 15.0 mmol) was added at 0° C. and the mixture was stirred at 20° C. for 12 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 40 g AgelaFlash® Silica Flash Column, EtOA/MeOH with MeOH from 0˜10%, Flow Rate: 30 mL/min, 254 nm) to afford N-[1-(1,3-benzothiazol-5-yl)-2-nitro-ethyl]-2-methyl-propane-2-sulfinamide (3 g, 61.0% yield) as a yellow oil.

### Step 4: Synthesis of N-[2-amino-1-(1,3-benzothiazol-5-yl)ethyl]-2-methyl-propane-2-sulfinamide

A mixture of N-[1-(1,3-benzothiazol-5-yl)-2-nitro-ethyl]-2-methyl-propane-2-sulfinamide (3 g, 9.16 mmol), NiCl2-6H₂O (2.19 g, 9.20 mmol) and MeOH (20 mL) was cooled to 0° C. then NaBH4 (1.74 g, 46.1 mmol) was added. The mixture was stirred at 0° C. for 2 hours. The resulting mixture was filtered and washed with water. The filtrate was lyophilized to dryness and the residue was triturated with DCM (100 mL). The precipitate was removed by filtration, washed with DCM (50 mL*2) and the combined filtrate was concentrated under reduced pressure to afford N-[2-amino-1-(1,3-benzothiazol-5-yl)ethyl]-2-methyl-propane-2-sulfinamide (1.7 g, 62.4% yield) as yellow oil. LCMS (ESI) [M+H]⁺ m/z: calcd 298.0, found 298.0.

### Step 5: Synthesis of N—[1-(1,3-benzothiazol-5-yl)-2-[(2,2-dimethoxy-1-methyl-ethyl)amino]ethyl]-2-methyl-propane-2-sulfinamide

A mixture of N-[2-amino-1-(1,3-benzothiazol-5-yl)ethyl]-2-methyl-propane-2-sulfinamide (1.7 g, 5.72 mmol), 1,1-dimethoxypropan-2-one (1.70 g, 14.4 mmol), Na₂SO₄ (2.89 g, 20.4 mmol), AcOH (1.70 g, 6.97 mmol) and DCE (20 mL) was stirred at 20° C. for 12 hours. NaBH4 (680 mg, 18.0 mmol) was added and the mixture was stirred at 20° C. for 1 hour. The resulting mixture was quenched by addition of water (50 mL) and extracted with DCM (100 mL*3). The combined organic layer was washed with saturated NH₄Cl aqueous solution (50 mL*2), brine (50 mL), dried over anhydrous Na₂SO₄, filtered and concentrated under reduced pressure to give N-[1-(1,3-benzothiazol-5-yl)-2-[(2,2-dimethoxy-1-methyl-ethyl)amino]ethyl]-2-methyl-propane-2-sulfinamide (1.7 g, 74.4% yield) as yellow oil.

### Step 6: Synthesis of ditert-butyl 2-(1,3-benzothiazol-5-yl)-5-methyl-piperazine-1,4-dicarboxylate

A mixture of N-[1-(1,3-benzothiazol-5-yl)-2-[(2,2-dimethoxy-1-methyl-ethyl)amino]ethyl]-2-methyl-propane-2-sulfinamide (1.5 g, 3.75 mmol) and 12N HCl/H₂O (30.0 mL, 0.36 mol) was stirred at 20° C. for 30 minutes. The mixture was concentrated under reduced pressure and the residue was dissolved in MeOH (2 mL), followed by addition of NaBH3(CN) (600 mg, 9.55 mmol) and Na₂SO₄ (1.80 g, 12.7 mmol). The mixture was stirred at 20° C. for 12.5 hours. Then the mixture was concentrated under reduced pressure and the residue was dissolved in THF (3 mL) and H₂O (1 mL), followed by addition of tert-butyl carbonate (3 mL, 13.1 mmol) and K₂CO₃ (1.59 g, 11.5 mmol). The mixture was stirred at 20° C. for 2 hours. The resulting mixture was quenched by addition of water (100 mL) and extracted with EtOAc (100 mL*3). The combined organic layer was washed with saturated NH₄Cl aqueous solution (100 mL*2), brine (100 mL), dried over anhydrous Na₂SO₄, filtered and concentrated under reduced pressure to give a residue which was purified by flash chromatography (ISCO®; 12 g AgelaFlash® Silica Flash Column, petroleum ether/EtOAc with EtOAc from 0˜20%, Flow Rate: 30 mL/min, 254 nm) to afford ditert-butyl 2-(1,3-benzothiazol-5-yl)-5-methyl-piperazine-1,4-dicarboxylate (900 mg, 55.3% yield) as yellow oil.

### Step 7: Synthesis of 5-(5-methylpiperazin-2-yl)-1,3-benzothiazole

A mixture of ditert-butyl 2-(1,3-benzothiazol-5-yl)-5-methyl-piperazine-1,4-dicarboxylate (800 mg, 1.85 mmol), DCM (5 mL) and 4M HCl/EtOAc (5 mL, 20 mmol) was stirred at 20° C. for 1 hour. The mixture was concentrated under reduced pressure and the residue was purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %);Mobile phase B: MeCN; Gradient: B from 5% to 35% in 9.5 min, hold 100% B for 2.5 min; Flow Rate: 25 mL/min; Column Temperature: 30° C.; Wavelength: 220 nm, 254 nm) to afford 5-(5-methylpiperazin-2-yl)-1,3-benzothiazole (100 mg, 23.2% yield) as white solid.

### Step 8: Synthesis of 1-[5-(1,3-benzothiazol-5-yl)-2-methyl-piperazin-1-yl]-2-methyl-propan-1-one

To a mixture of 5-(5-methylpiperazin-2-yl)-1,3-benzothiazole (100 mg, 0.428 mmol), TEA (72.6 mg, 0.717 mmol) and DCM (2 mL) was added 2-methylpropanoyl chloride (27 mg, 0.253 mmol) and the mixture was stirred at 20° C. for 1 hour. The mixture was concentrated under reduced pressure to give a 1-[5-(1,3-benzothiazol-5-yl)-2-methyl-piperazin-1-yl]-2-methyl-propan-1-one (120 mg, crude) as yellow oil.

### Step 9: Synthesis of N-(6-amino-5-methyl-3-pyridyl)-2-[2-(1,3-benzothiazol-5-yl)-5-methyl-4-(2-methylpropanoyl)piperazin-1-yl]-2-oxo-acetamide

To a mixture of 2-[(6-amino-5-methyl-3-pyridyl)amino]-2-oxo-acetic acid (30 mg, 0.154 mmol), 1-[5-(1,3-benzothiazol-5-yl)-2-methyl-piperazin-1-yl]-2-methyl-propan-1-one (60.0 mg, 0.198 mmol), HATU (55 mg, 0.145 mmol) and DMF (2 mL) was added DIPEA (0.574 mmol, 0.1 mL) and the mixture was stirred at 20° C. for 2 hours. The mixture was purified by preparative HPLC (Instrument: Gilson GX-281 Liquid Handler, Gilson 322 Pump, Gilson 156 UV Detector; Column: Waters Xbridge 150×25 mm×5 m; Mobile phase A: H₂O with 0.05% NH₃—H₂O (v %);Mobile phase B: MeCN; Gradient: B from 11% to 41% in 9.5 min, hold 100% B for 2.5 min; Flow Rate: 25 mL/min; Column Temperature: 30° C.; Wavelength: 220 nm, 254 nm) to afford N-(6-amino-5-methyl-3-pyridyl)-2-[2-(1,3-benzothiazol-5-yl)-5-methyl-4-(2-methylpropanoyl)piperazin-1-yl]-2-oxo-acetamide (35 mg, 47.4% yield) as yellow oil.

### Step 10: Synthesis of 2-[(2S,5R)-4-acetyl-2-(1, 3-benzothiazol-5-yl)-5-methyl-piperazin-1-yl]-N-(6-amino-5-methyl-3-pyridyl)-2-oxo-acetamide

N-(6-amino-5-methyl-3-pyridyl)-2-[2-(1,3-benzothiazol-5-yl)-5-methyl-4-(2-methylpropanoyl)piperazin-1-yl]-2-oxo-acetamide (35 mg, 72.8 μmol) was separated by chiral SFC (Instrument: Thar 800Q; Daicel Chiralpak IG (250 mm*30 mm, 10 μm); Mobile phase: supercritical C₀₂/EtOH (0.1% NH₃—H₂O, v %)=75/25; Flow Rate: 80 mL/min; Column Temperature: 38° C.; Nozzle Pressure: 100 bar; Nozzle Temperature: 60° C.; Evaporator Temperature: 20° C.; Trimmer Temperature: 25° C.; Wavelength: 220 nm) to give Compound 43(peak 2, retention time=3.127 min) and Compound 45 (peak 3, retention time=3.383 min).

Compound 43: N-(6-amino-5-methyl-3-pyridyl)-2-[(2S,5R)-2-(1,3-benzothiazol-5-yl)-5-methyl-4-(2-methylpropanoyl)piperazin-1-yl]-2-oxo-acetamide (12 mg, single unknown enantiomer with trans relative chemistry, peak 2, retention time=3.127 min, white solid). ¹H NMR (400 MHz, DMSO-d₆) δ ppm 10.34-10.74 (m, 1H), 9.33-9.45 (m, 1H), 8.12-8.23 (m, 1H), 7.78-8.11 (m, 2H), 7.54 (br d, J=2.5 Hz, 2H), 5.57-5.90 (m, 2H), 5.03-5.52 (m, 1H), 4.00-4.63 (m, 2H), 3.62-3.90 (m, 1H), 2.63-2.87 (m, 3H), 1.92-2.11 (m, 3H), 1.26-1.35 (m, 2H), 0.66-1.18 (m, 7H); LCMS(ESI) [M+H]⁺ m/z: calcd 481.1, found 481.1; HPLC: 100%®220 nm, 100%®254 nm; 98.9% ee.

Compound 45: N-(6-amino-5-methyl-3-pyridyl)-2-[(2S,5R)-2-(1,3-benzothiazol-5-yl)-5-methyl-4-(2-methylpropanoyl)piperazin-1-yl]-2-oxo-acetamide (12 mg, single unknown enantiomer with trans relative chemistry, peak 3, retention time=3.383 min, white solid). ¹H NMR (400 MHz, DMSO-d₆) δ ppm 10.50-10.91 (m, 1H), 9.42-9.52 (m, 1H), 8.20-8.28 (m, 1H), 7.86-8.17 (m, 2H), 7.18-7.66 (m, 2H), 5.95 (br d, J=16.3 Hz, 2H), 5.07-5.70 (m, 1H), 4.07-4.70 (m, 2H), 3.68-3.98 (m, 1H), 3.21-3.29 (m, 1H), 2.69-2.95 (m, 2H), 1.96-2.17 (m, 3H), 1.33-1.45 (m, 2H), 1.21 (br d, J=6.3 Hz, 1H), 0.89-1.12 (m, 5H), 0.71-0.83 (m, 1H); LCMS(ESI) [M+H]⁺ m/z: calcd 481.1, found 481.1; HPLC: 100%®220 nm, 100%®254 nm; 98.5% ee.

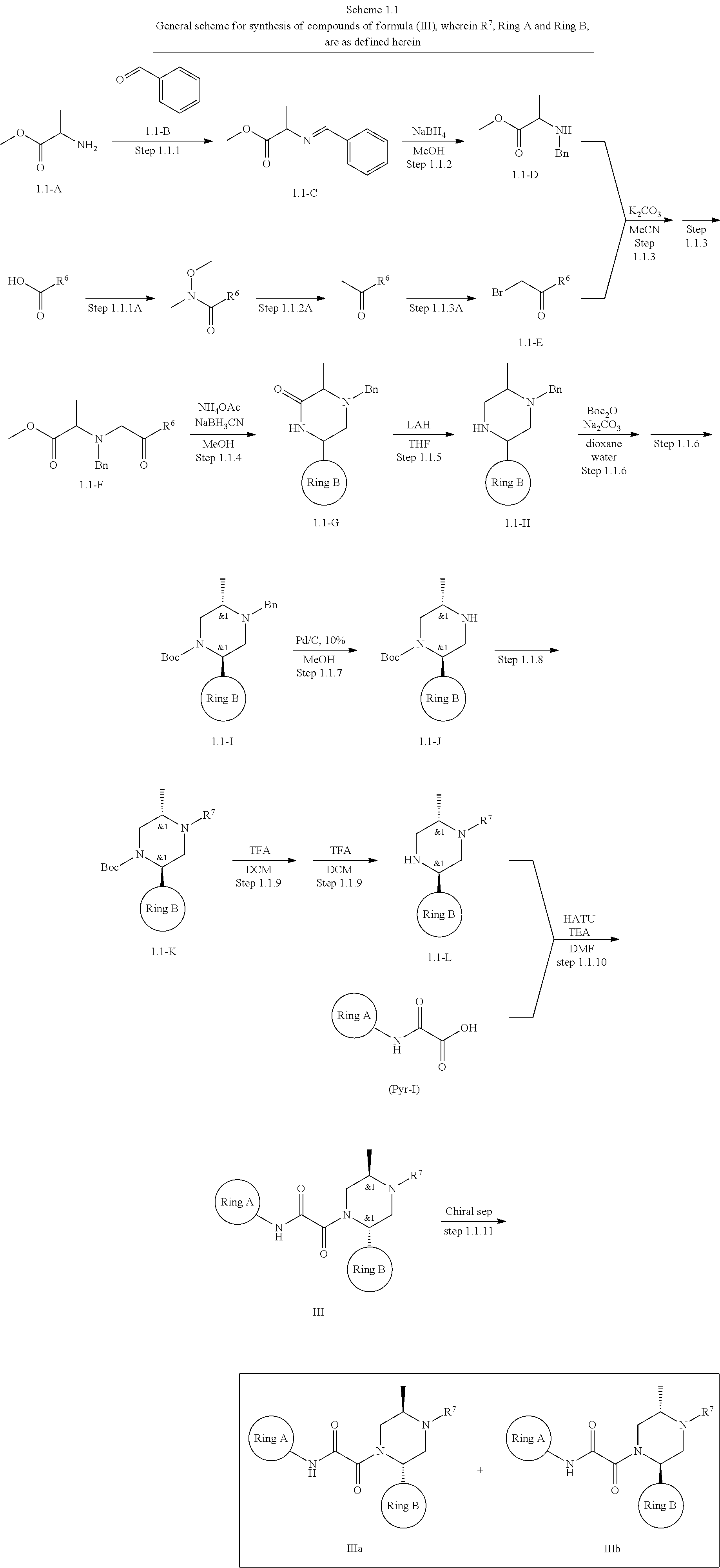

Scheme 1.1 General Procedures

### Step 1.1.1: Synthesis of 1.1-C

Methyl 2-aminopropanoate (1.1-A)(1 eq, HCl) and benzaldehyde (1 eq) were mixed together in DCM and TEA (1.2 eq) was added thereto. The resulting mixture was stirred overnight. The reaction mixture was washed with water, dried over Na₂SO₄, filtered and evaporated to obtain methyl 2-[(E)-benzylideneamino]propanoate (1.1-C).

### Step 1.1.2: Synthesis of 1.1-D

Methyl 2-[(E)-benzylideneamino]propanoate (1 eq) was dissolved in MeOH and the resulting solution was cooled to 0° C. in an ice bath. Sodium borohydride (0.35 eq) was added portion wise to the previous solution. After addition completed, the reaction mixture was allowed to warm to rt and stirred overnight. Water (100 ml) was added to the reaction mixture and the resulting mixture was concentrated in vacuum. The residue was diluted with water and the resulting mixture was extracted with DCM twice, dried over Na₂SO₄, filtered and evaporated to obtain methyl 2-(benzylamino)propanoate (1.1-D).

### Step 1.1.A

To the stirred solution of acid (1 eq) in THF (25 mL) CDI (1.2 eq) was added and the reaction mixture was stirred at 25° C. for 1.5 hr. N-Methoxymethanamine (1.3 eq, HCl) was added followed by addition of TEA (1.3 eq). The reaction mixture was stirred at 25° C. for 17 hr. THF was evaporated. The residue was diluted with DCM and washed with water 3 times. Organic layer was dried over Na₂SO₄. DCM was evaporated to give respective Weinreb amide which was used in the next step without further purification.

### Step 1.1.2A

To the stirred solution of respective Weinreb amide (1 eq) in THF (15 mL) under Ar atmosphere methylmagnesium bromide, 3M in ether (1.5 eq) was added dropwise. The reaction mixture was the stirred at 25° C. for 18 hr. The reaction was quenched with water (dropwise) and product was extracted with EtOAc twice. Combined organic layers were washed with water dried over Na₂SO₄. Solvent was evaporated to give crude Ac—R⁶ which was used in the next step without further purification.

### Step 1.1.3A: Synthesis of 1.1-E

Ac—R⁶ was dissolved in AcOH and bromine (1.1 eq) was added dropwise. The reaction mixture was stirred at rt overnight. Water was added and it was washed with DCM, organic phase was collected an washed brine, dried Na₂SO₄ and concentrated to afford 1.1-E.

### Step 1.1.3: Synthesis of 1.1-F

1.1-D (1 eq) was dissolved in MeCN and potassium carbonate—granular (1.1 eq) was added thereto followed by addition of 1.1-E (1 eq). The resulting mixture was vigorously stirred overnight. The reaction mixture was concentrated in vacuum and water was added to the residue. The resulting mixture was extracted with MTBE and combined organic layers were dried over Na₂SO₄, filtered and evaporated to obtain 1.1-F which was used further without purification.

### Step 1.1.4: Synthesis of 1.1-G

1.1-F (1 eq) was dissolved in MeOH and ammonium acetate (10 eq) was added followed by addition of sodium cyanoborohydride (1.2 eq). The resulting mixture was stirred for 1 hr and then heated to reflux and refluxed overnight. The reaction mixture was cooled and concentrated in vacuum. The residue was basified by addition of aq. K₂CO₃. The resulting mixture was extracted with MTBE and combined organic layers were dried over Na₂SO₄, filtered and evaporated. The residue was purified by column chromatography (EtOAc:Hexane from 2:1 to 5:1) to obtain 1.1-G.

### Step 1.1.5: Synthesis of 1.1-H

LAH (3 eq) was suspended in THF and the resulting suspension was heated to reflux. A solution of 1.1-G (1 eq) in THF was added dropwise to the previous suspension, maintaining a gentle reflux. After addition completed, the reaction mixture was refluxed for 4 hr and then allowed to cool to rt and stirred overnight. Water was carefully added dropwise to the precooled reaction mixture followed by addition of a aq. KOH solution and water. The resulting mixture was stirred for 30 min and filtered. A filter cake was rinsed with THF and the filtrate was concentrated in vacuum to obtain 1.1-H.

### Step 1.1.6: Synthesis of 1.1-I

1.1-H (1 eq) was dissolved in dioxane and sodium carbonate (2 eq) was added thereto followed by addition of water. Di-tert-butyl dicarbonate (2 eq) was added dropwise to the resulting mixture. The reaction mixture was stirred overnight. The reaction mixture was concentrated in vacuum. The residue was diluted with water and the resulting mixture was extracted with EtOAc twice. Combined organic layers were washed with brine, dried over Na₂SO₄, filtered and evaporated. The residue was purified by column chromatography eluting with a mixture of hexane and EtOAc (15:1) to obtain 1.1-I.

### Step 1.1.7: Synthesis of 1.1-J

1.1-J (1 eq) was dissolved in MeOH and palladium, 10% on carbon, Type 487, dry (0.4 eq) was added thereto. The reaction mixture was evacuated and backfilled three times with hydrogen and was hydrogenated at 1 atm (balloon) overnight. The catalyst was filtered off and the filtrate was concentrated in vacuum to obtain 1.1-J.

### Step 1.1.8A: Synthesis of 1.1-K

1.1-J (1 eq) and TEA (3 eq) were mixed together in DCM and the resulting solution was cooled to −5° C. in an ice/MeOH bath. (R^{x}CO)₂O (1 eq) or RsCl (1.3 eq) was added dropwise to the previous solution and the resulting mixture was allowed to warm to rt and stirred overnight. The reaction mixture was diluted with DCM and the resulting solution was washed with water twice, dried over Na₂SO₄, filtered and evaporated to obtain 1.1-K.

### Step 1.1.8B: Synthesis of 1.1-K

1.1-J (1 eq) was dissolved in DCM and acetic acid (1 eq) was added thereto followed by addition of aldehyde (1.5 eq). Sodium triacetoxyborohydride (2 eq) was added to the previous mixture and the resulting mixture was stirred overnight. NaHCO₃ aq. solution was added to the reaction mixture and the resulting mixture was extracted with DCM twice. Combined organic layers were dried over Na₂SO₄, filtered and evaporated to obtain product 1.1-K.

### Step 1.1.8C: Synthesis of 1.1-K

1.1-J (1 eq) was dissolved in DCM and aldehyde (1.5 eq) was added thereto followed by addition of AcOH (1 eq). Sodium cyanoborohydride (2 eq) was added to the previous mixture and the resulting mixture was stirred overnight. NaHCO₃ aq. solution was added to the reaction mixture and the resulting mixture was extracted with DCM twice. Combined organic layers were dried over Na₂SO₄, filtered and evaporated to obtain 1.1-K (crude).

### Step 1.1.8D: Synthesis of 1.1-K

1.1-J (1 eq) was dissolved in DCM and TEA (3 eq) was added thereto. The resulting mixture was cooled to 0° C. and R-LG (1 eq) was added dropwise. The resulting mixture was allowed to warm to rt and stirred overnight. The reaction mixture was diluted with DCM and the resulting mixture was washed with water twice, dried over Na₂SO₄, filtered and evaporated to obtain product 1.1-K.

### Step 1.1.8E: Synthesis of 1.1-K

Oxalyl chloride (1.2 eq) was added in the solution of R⁵OH (1 eq) in chloroform (15 mL) followed by addition of 1-2 drop of DMF. The resulting mixture was mixed 2 hr at rt. Then reaction mixture was washed by NaHCO₃saturated aqueous solution (3*30 ml.) to neutral pH, dried over Na₂SO₄, filtered and added at 0° C. to the stirred solution of 1.1J (1 eq) and TEA (2 eq) in chloroform (15 mL). Reaction was warmed to 25° C. and stirred 16 hr at 25° C. Reaction mixture was washed by water twice, dried over Na₂SO₄ and evaporated to dryness under reduced pressure to give 1.1-K.

### Step 1.1.9: Synthesis of 1.1-L

1.1-K (1 eq) was dissolved in DCM and TFA (15 eq) was added thereto. The resulting mixture was stirred at 25° C. for 1 hr, and then evaporated in vacuum. Crushed ice (15 g) was added to the residue and pH was adjusted to 10 with a 7M aqueous solution of K₂CO₃. The resulting mixture was extracted with DCM. The combined organic extracts were dried over sodium sulphate and evaporated in vacuum to afford 1.1-L.

### Step 1.1.1A: Synthesis of Product III

1.1-L (1 eq), Pyr-I (1 eq) and TEA (2.5 eq+1.0 eq per each Pyr-I eq, if amine salt used) were mixed together in DMF. HATU (1.5 eq) was added thereto and the resulting mixture was stirred overnight. The reaction mixture was concentrated in vacuum and the residue was purified by HPLC to obtain product compound III.

### Step 1.1 LOB: Synthesis of product III

DIPEA (2.5 eq+1.0 eq per each acid eq, if amine salt used) was added to the solution of respective amine or it salt (1.1-L) (1.0 eq) and Pyr-I (1 eq) in DMF. The resulting mixture was stirred for 5 min followed by the addition of the solution of HATU (1.1 eq) in DMF. Then, the reaction mixture was stirred overnight at rt. After the completion of the reaction, monitored by LCMS, the resulting suspension was concentrated under reduced pressure. The obtained filtrate was subjected to HPLC (Waters SunFire C18 19*100 5 mkm column and H₂O-MeOH as a mobile phase) to afford pure product (III).

### Boc Group Deprotection Methods

### Method A:

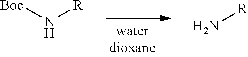

Boc-protected reactant was dissolved in a mixture of dioxane-water (1:1). The resulting mixture was heated at 100° C. overnight. The reaction mixture was concentrated in vacuum. The residue was purified by HPLC to obtain pure product.

### Method B:

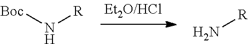

A solution of Boc-protected reactant in Et20/HCl (1:10) was stirred at rt for 12 hr. Resulting solid was collected by filtration, then washed with MTBE. The residue was purified by HPLC to obtain pure product.

### Method C:

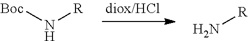

A solution of Boc-protected reactant in diox/HCl (1:10) was stirred at rt for 12 hr. Resulting solid was collected by filtration. The residue was purified by HPLC to obtain pure product.

### Method D:

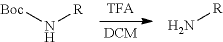

A solution of Boc-protected reactant was dissolved in DCM and TFA (1:10) was added thereto. The resulting mixture was stirred for 1 hr. The reaction mixture was carefully poured into K₂CO₃ solution (25 g in 100 ml of water) and the resulting mixture was extracted with DCM twice. Combined organic layers were dried over Na₂SO₄, filtered and concentrated in vacuum to obtain pure product.

### Example 13. Compound 20 N-(6-amino-5-methylpyridin-3-yl)-2-(2-(4-fluorophenyl)-4-isobutyryl-5-methylpiperazin-1-yl)-2-oxoacetamide

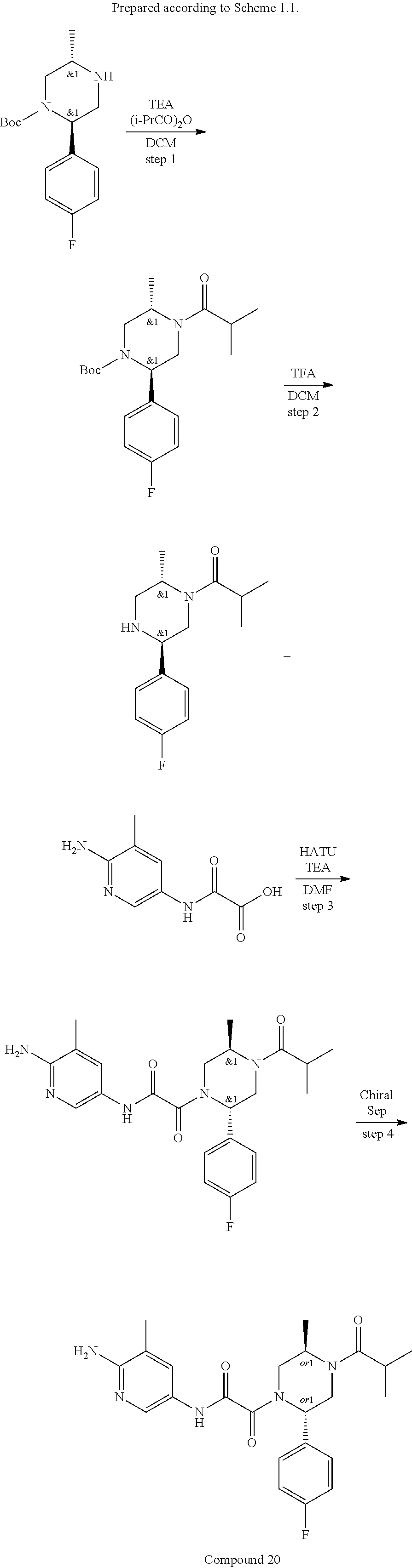 [SMILES: C[C@H]1CN(C(=O)OC(C)(C)C)[C@H](c2ccc(F)cc2)CN1 ; CC(C)C(=O)N1C[C@@H](c2ccc(F)cc2)N(C(=O)OC(C)(C)C)C[C@@H]1C ; CC(C)C(=O)N1C[C@@H](c2ccc(F)cc2)NC[C@@H]1C ; Cc1cc(NC(=O)C(=O)O)cnc1N ; Cc1cc(NC(=O)C(=O)N2C[C@@H](C)N(C(=O)C(C)C)C[C@@H]2c2ccc(F)cc2)cnc1N ; Cc1cc(NC(=O)C(=O)N2C[C@@H](C)N(C(=O)C(C)C)C[C@@H]2c2ccc(F)cc2)cnc1N]

### Step 1. Synthesis of rac-(2R,5S)-tert-butyl 2-(4-fluorophenyl)-4-isobutyryl-5-methylpiperazine-1-carboxylate

Prepared as described in Scheme 1.1 and with (i—PrCO)₂O. Yield: 611 mg of crude (85% by NMR). LCMS(ESI): [M-Boc]⁺ m/z: calcd 264.2; found 265.2; Rt=1.492 min.

### Step 2. Synthesis of rac-1-((2R,5S)-5-(4-fluorophenyl)-2-methylpiperazin-1-yl)-2-methylpropan-1-one

Prepared as described in Scheme 1.1. Yield: 394 mg (88.91%). LCMS(ESI): [M]⁺ m/z: calcd 264.2; found 265.2; Rt=0.840 min.

### Step 3. Synthesis of N-(6-amino-5-methylpyridin-3-yl)-2-(2-(4-fluorophenyl)-4-isobutyryl-5-methylpiperazin-1-yl)-2-oxoacetamide

Prepared as described in Scheme 1.1. Yield: 104.3 mg (15.85%). HPLC conditions: Column: SunFire 100*19 mm, 5 microM; 2-10 min 40-55% MeOH+NH₃ 30 ml/min, loading pump 4 ml/min MeOH.

Compound 157: ¹H NMR (600 MHz, DMSO-d₆) δ (ppm) 0.86 (m, 6H), 1.16 (m, 3H), 1.99 (m, 4H), 2.73 (m, 2H), 3.66 (m, 1H), 4.21 (m, 1H), 4.92 (m, 1H), 5.57 (m, 3H), 7.17 (m, 2H), 7.27 (m, 1H), 7.39 (m, 1H), 7.50 (m, 1H), 8.02 (m, 1H), 10.57 (m, 1H).

LCMS(ESI): [M]⁺ m/z: calcd 441.2; found 442.2; Rt=0.992 min.

### Step 4. Chiral Separation

Racemic N-(6-amino-5-methyl-3-pyridyl)-2-[(2S,5R)-2-(4-fluorophenyl)-5-methyl-4-(2-methyl propanoyl)piperazin-1-yl]-2-oxo-acetamide (95 mg, 215.18 umol) was chirally separated (Column: Chiralpak AD-H III(250*20 mm, 5mkm); Mobile phase: IPA-MeOH 50-50 Flow Rate: 12 mL/min; m=0.09r, 3 injections, 30 mg/inj., 4.2 hr) to obtain crude product which was re-purified (Column: Chiralcel OD-H (250*20 mm, 5mkm); Mobile phase: Hexane-IPA-MeOH 80-10-10 Flow Rate: 12 mL/min; m=0.045 g, 4 injections, 11 mg/inj., 2.5 hr) to obtain N-(6-amino-5-methyl-3-pyridyl)-2-[(2S,5R)-2-(4-fluorophenyl)-5-methyl-4-(2-methylpropanoyl)piperazin-1-yl]-2-oxo-acetamide (27.63 mg, 62.58 umol, 29.08% yield) (RT=16.48) and N-(6-amino-5-methyl-3-pyridyl)-2-[(2R,5S)-2-(4-fluorophenyl)-5-methyl-4-(2-methylpropanoyl)piperazin-1-yl]-2-oxo-acetamide (25.06 mg, 56.76 umol, 26.38% yield) (RT=29.84).

Rel Time for Compound 20 in analytical conditions (column: OD-H, Hexane-IPA-MeOH, 80-10-10, 0.6 ml/min as mobile phase) 29.82 min and for Compound 20 25.27 min.

Retention time: 25.27 min

¹H NMR (600 MHz, DMSO-d₆) δ (ppm) 1.00 (m, 9H), 2.02 (m, 3H), 2.85 (m, 2H), 3.61 (m, 1H), 3.95 (m, 2H), 4.83 (m, 1H), 5.63 (m, 3H), 7.24 (m, 4H), 7.50 (m, 1H), 8.02 (m, 1H), 10.58 (m, 1H).

LCMS(ESI): [M]⁺ m/z: calcd 441.2; found 442.2; Rt=2.357 min.

### Example 14. Compound 157 rac-N-(6-amino-5-methylpyridin-3-yl)-2-(2-(4-fluorophenyl)-4-isobutyryl-5-methylpiperazin-1-yl)-2-oxoacetamide

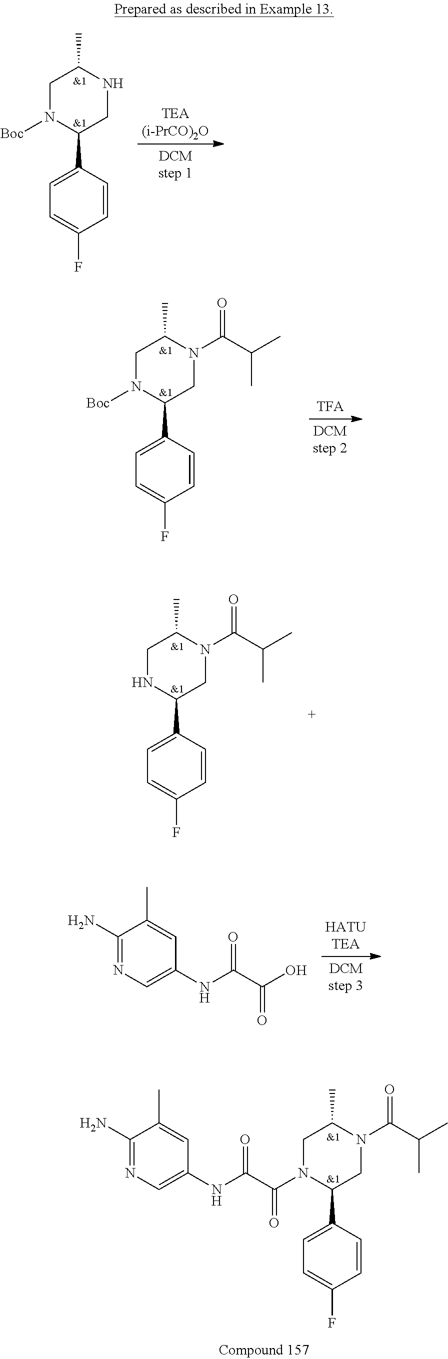 [SMILES: C[C@H]1CN(C(=O)OC(C)(C)C)[C@H](c2ccc(F)cc2)CN1 ; CC(C)C(=O)N1C[C@@H](c2ccc(F)cc2)N(C(=O)OC(C)(C)C)C[C@@H]1C ; CC(C)C(=O)N1C[C@@H](c2ccc(F)cc2)NC[C@@H]1C ; Cc1cc(NC(=O)C(=O)O)cnc1N ; Cc1cc(NC(=O)C(=O)N2C[C@H](C)N(C(=O)C(C)C)C[C@H]2c2ccc(F)cc2)cnc1N]

¹H NMR (600 MHz, DMSO-d₆) δ (ppm) 0.86 (m, 6H), 1.16 (m, 3H), 1.99 (m, 4H), 2.73 (m, 2H), 3.66 (m, 1H), 4.21 (m, 1H), 4.92 (m, 1H), 5.57 (m, 3H), 7.17 (m, 2H), 7.27 (m, 1H), 7.39 (m, 1H), 7.50 (m, 1H), 8.02 (m, 1H), 10.57 (m, 1H).

LCMS(ESI): [M]⁺ m/z: calcd 441.2; found 442.2; Rt=0.992 min.

### Example 15. Compound 83 and Compound 88 5-(2-(2-(4-fluorophenyl)-4-isobutyryl-5-methylpiperazin-1-yl)-2-oxoacetamido)-2-methoxynicotinamide

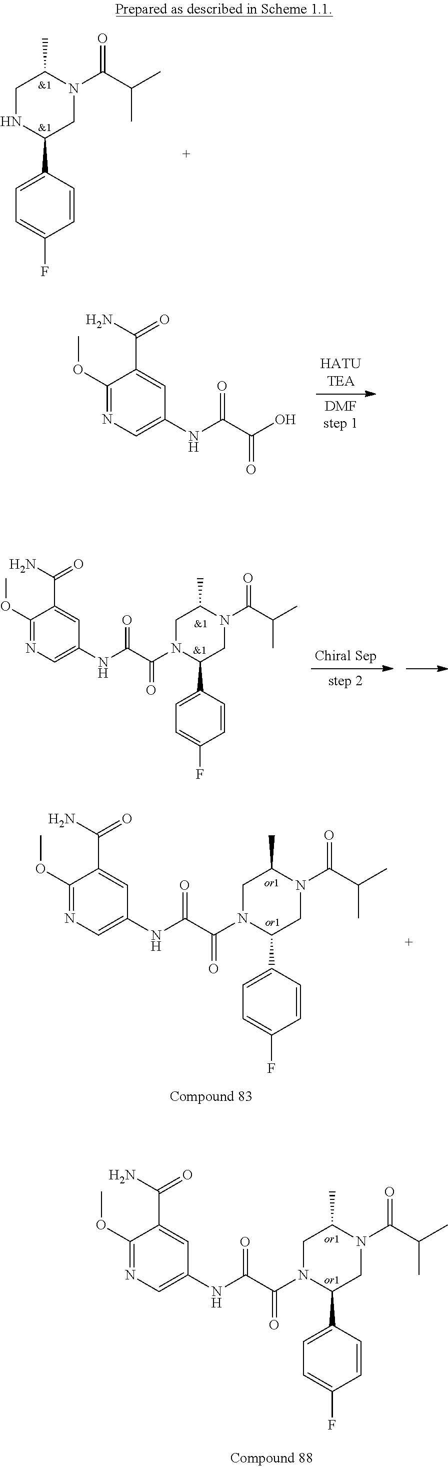 [SMILES: CC(C)C(=O)N1C[C@@H](c2ccc(F)cc2)NC[C@@H]1C ; COc1ncc(NC(=O)C(=O)O)cc1C(N)=O ; COc1ncc(NC(=O)C(=O)N2C[C@H](C)N(C(=O)C(C)C)C[C@H]2c2ccc(F)cc2)cc1C(N)=O ; COc1ncc(NC(=O)C(=O)N2C[C@@H](C)N(C(=O)C(C)C)C[C@@H]2c2ccc(F)cc2)cc1C(N)=O ; COc1ncc(NC(=O)C(=O)N2C[C@H](C)N(C(=O)C(C)C)C[C@H]2c2ccc(F)cc2)cc1C(N)=O]

### Step 1: 5-(2-(2-(4-fluorophenyl)-4-isobutyryl-5-methylpiperazin-1-yl)-2-oxoacetamido)-2-methoxynicotinamide

Prepared as described in Scheme 1.1, step 1.1.10A. Yield: 0.23 g (29.82%).

HPLC conditions: Column:SunFire 100*19 mm, 5 microM; 2-10 min 40-50% MeCN 30 ml/min (loading pump 4 ml MeCN).

LCMS(ESI): [M]⁺ m/z: calcd 485.2; found 486.2; Rt=1.136 min.

### Step 2: Chiral Separation

Racemic 5-[[2-[(2S,5R)-2-(4-fluorophenyl)-5-methyl-4-(2-methylpropanoyl)piperazin-1-yl]-2-oxo-acetyl]amino]-2-methoxy-pyridine-3-carboxamide (0.23 g, 473.73 umol) was chirally separated (Column: Chiralpak IA-I (250*4.6, 5 mkm), IPA-MeOH,50-50, 12 ml/min) to obtain 5-[[2-[(2S,5R)-2-(4-fluorophenyl)-5-methyl-4-(2-methylpropanoyl)piperazin-1-yl]-2-oxo-acetyl]amino]-2-methoxy-pyridine-3-carboxamide (0.11 g, 226.57 umol, 47.83% yield) and 5-[[2-[(2R,5S)-2-(4-fluorophenyl)-5-methyl-4-(2-methylpropanoyl)piperazin-1-yl]-2-oxo-acetyl]amino]-2-methoxy-pyridine-3-carboxamide (0.1123 g, 231.30 umol, 48.83% yield).

Rel Time for Compound 83 in analytical conditions (column: IA, IPA-MeOH, 50-50, 0.6 ml/min as mobile phase) 17.44 min and for Compound 88 73.45 min.

Compound 83: Retention time: 17.44 min

¹H NMR (600 MHz, DMSO-d₆) δ (ppm) 11.17-10.82 (m, 1H), 8.56-8.32 (m, 2H), 7.78-7.66 (m, 2H), 7.43-7.26 (m, 2H), 7.24-7.11 (m, 2H), 5.72-5.34 (m, 1H), 4.96-4.02 (m, 2H), 3.97-3.91 (m, 3H), 3.81-3.62 (m, 1H), 3.26-2.64 (m, 3H), 1.27-1.10 (m, 3H), 1.01-0.90 (m, 3H), 0.89-0.72 (m, 3H).

LCMS(ESI): [M]⁺ m/z: calcd 485.2; found 486.2; Rt=2.747 min.

Compound 88: Retention time: 73.45 min

1H NMR (600 MHz, DMSO-d₆) δ (ppm) 11.20-10.77 (m, 1H), 8.57-8.29 (m, 2H), 7.78-7.66 (m, 2H), 7.43-7.27 (m, 2H), 7.24-7.12 (m, 2H), 5.70-5.33 (m, 1H), 4.95-4.03 (m, 2H), 3.98-3.90 (m, 3H), 3.80-3.63 (m, 1H), 3.26-2.65 (m, 3H), 1.27-1.10 (m, 3H), 1.01-0.90 (m, 3H), 0.89-0.70 (m, 3H).

LCMS(ESI): [M]⁺ m/z: calcd 485.2; found 486.2; Rt=2.748 min.

### Example 16. Compound 80 and Compound 154 N-(6-amino-5-methylpyridin-3-yl)-2-(2-(4-fluorophenyl)-4-isobutyl-5-methylpiperazin-1-yl)-2-oxoacetamide

Prepared as described in Scheme 1.1

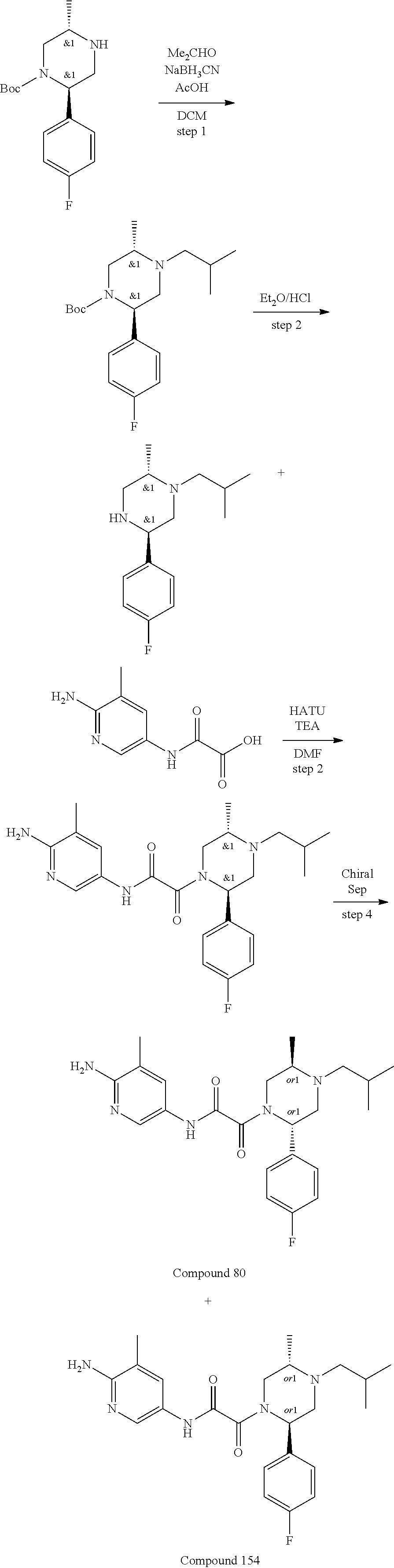

### Step 1: Synthesis of rac-(2R,5S)-tert-butyl 2-(4-fluorophenyl)-4-isobutyl-5-methylpiperazine- PGP424,C_{3 1}-carboxylate

Prepared as described in Scheme 1.1, step 1.1.8C. Yield: 195 mg of crude (80% by NMR). LCMS(ESI): [M]⁺ m/z: calcd 350.2; found 351.2; Rt=0.30 min.

### Step 2: Synthesis of rac-(2R,5S)-5-(4-fluorophenyl)-1-isobutyl-2-methylpiperazine Prepared by method B of “Boc Deprotection methods”. Yield: 210 mg (HCl; crude)

LCMS(ESI): [M]⁺ m/z: calcd 250.2; found 251.2; Rt=0.863 min.

### Step 3: Synthesis of N-(6-amino-5-methylpyridin-3-yl)-2-(2-(4-fluorophenyl)-4-isobutyl-5-methylpiperazin-1-yl)-2-oxoacetamide

Prepared as described in Scheme 1.1, step 1.1.10A. Yield: 59.9 mg (21.57%).

LCMS(ESI): [M]⁺ m/z: calcd 427.2; found 428.2; Rt=0.855 min.

### Step 4: Chiral Separation

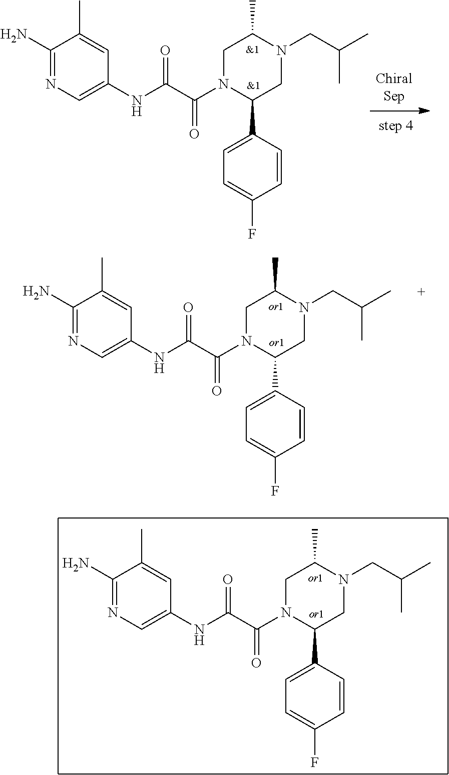

Racemic N-(6-amino-5-methyl-3-pyridyl)-2-[(2S,5R)-2-(4-fluorophenyl)-4-isobutyl-5-methyl-piperazin-1-yl]-2-oxo-acetamide (94.5 mg, 245.18 umol) was chirally separated (Column: Chiralpak ICIII (250*30, 5mkm), Hexane-IPA-MeOH, 80-10-10, 12 ml/min) to obtain N-(6-amino-5-methyl-3-pyridyl)-2-[(2S,5R)-2-(4-fluorophenyl)-4-isobutyl-5-methyl-piperazin-1-yl]-2-oxo-acetamide (22.97 mg, 53.73 umol, 38.35% yield) (RT=29.95 min) and N-(6-amino-5-methyl-3-pyridyl)-2-[(2R,5S)-2-(4-fluorophenyl)-4-isobutyl-5-methyl-piperazin-1-yl]-2-oxo-acetamide (22.94 mg, 53.66 umol, 38.30% yield) (RT=40.82 min).

Rel Time for Compound 154 in analytical conditions (column: IC, Hexane-IPA-MeOH, 60-20-20, 0.6 ml/min as mobile phase) 13.58 min and for Compound 8017.14 min.

Compound 80: Retention time: 17.14 min

¹H NMR (600 MHz, DMSO-d₆) δ (ppm) 0.80-0.83 (m, 4H), 0.93 (d, 3H), 1.70-1.74 (m, 2H), 2.02 (m, 3H), 2.11 (m, 2H), 2.82-309 (m, 4H), 3.50 (m, 1H), 3.99 (m, 1H), 5.08 (m, 1H), 5.46 (m, 1H), 5.69 (s, 2H), 7.14-7.19 (m, 2H), 7.49-7.56 (m, 2H), 8.00 (s, 1H), 10.51 (m, 1H). LCMS(ESI): [M]⁺ m/z: calcd 427.2; found 428.2; Rt=2.152 min.

Compound 154: Retention time: 13.58 min

1H NMR (600 MHz, DMSO-d₆) δ (ppm) 0.73-0.83 (m, 6H), 0.93 (d, 3H), 1.70-1.74 (m, 1H), 2.02 (s, 3H), 2.11 (m, 2H), 2.83 (m, 1H), 2.90 (m, 1H), 3.04-3.09 (m, 2H), 3.48 (m, 1H), 4.00 (m, 1H), 5.46 (m, 1H), 5.96 (m, 1H), 7.14-7.19 (m, 2H), 7.48-7.55 (m, 3H), 8.01 (s, 1H), 10.52 (s, 1H).

LCMS(ESI): [M]⁺ m/z: calcd 427.2; found 428.2; Rt=2.152 min.

### Example 17. Compound 86 N-(6-amino-5-cyclopropylpyridin-3-yl)-2-(2-(4-fluorophenyl)-4-isobutyryl-5-methylpiperazin-1-yl)-2-oxoacetamide

Prepared as described in Scheme 1.1

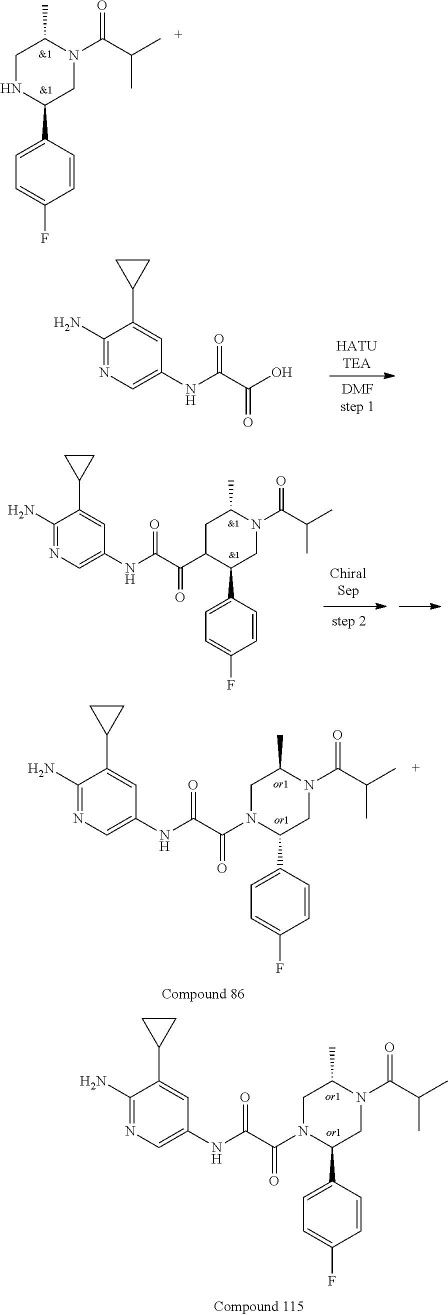 [SMILES: CC(C)C(=O)N1C[C@@H](c2ccc(F)cc2)NC[C@@H]1C ; Nc1ncc(NC(=O)C(=O)O)cc1C1CC1 ; CC(C)C(=O)N1C[C@@H](c2ccc(F)cc2)C(C(=O)C(=O)Nc2cnc(N)c(C3CC3)c2)C[C@@H]1C ; CC(C)C(=O)N1C[C@H](c2ccc(F)cc2)N(C(=O)C(=O)Nc2cnc(N)c(C3CC3)c2)C[C@H]1C ; CC(C)C(=O)N1C[C@@H](c2ccc(F)cc2)N(C(=O)C(=O)Nc2cnc(N)c(C3CC3)c2)C[C@@H]1C]

### Step 1: Synthesis of N-(6-amino-5-cyclopropylpyridin-3-yl)-2-(2-(4-fluorophenyl)-4-isobutyryl-5-methylpiperazin-1-yl)-2-oxoacetamide

Prepared according to Scheme 1.1, step 1.1.10A. Yield: 165 mg (37.32%).

HPLC conditions: Column:SunFire 100*19 mm, 5 microN; 2-10 min 25-40% MeOH 30 ml/min, loading pump 4 ml/min MeOH. LCMS(ESI): [M]⁺ m/z: calcd 467.2; found 468.2; Rt=1.022 min.

### Step 2: Chiral Separation

Racemic N-(6-amino-5-cyclopropyl-3-pyridyl)-2-[(2S,5R)-2-(4-fluorophenyl)-5-methyl-4-(2-methylpropanoyl)piperazin-1-yl]-2-oxo-acetamide (0.165 g, 352.92 umol) was chiral separated (Column: Chiralpak IA 250*20, 5 mkm; Hexane-IPA-MeOH, 60-20-20, 12 ml/min) to obtain N-(6-amino-5-cyclopropyl-3-pyridyl)-2-[(2S,5R)-2-(4-fluorophenyl)-5-methyl-4-(2-methylpropanoyl)piperazin-1-yl]-2-oxo-acetamide (49.04 mg, 104.89 umol, 29.72% yield) (RT=38.36 min) and N-(6-amino-5-cyclopropyl-3-pyridyl)-2-[(2R,5S)-2-(4-fluorophenyl)-5-methyl-4-(2-methylpropanoyl)piperazin-1-yl]-2-oxo-acetamide (65.4 mg, 139.88 umol, 39.63% yield) (RT=57.97 min).

Rel Time for Compound 115 in analytical conditions (column: IA, Hexane-IPA-MeOH, 50-5-25, 0.6 ml/min as mobile phase) 37.07 min and for Compound 86 24.36 min.

Compound 115: Retention time: 37.07 min ¹H NMR (600 MHz, DMSO-d₆) δ (ppm) 0.38-0.48 (m, 2H), 0.75 (dd, 1H), 0.85-0.88 (m, 3H), 0.88-0.93 (m, 2H), 0.97-1.01 (m, 2H), 1.09 (d, 1H), 1.21-1.25 (m, 2H), 1.60-1.70 (m, 1H), 2.65-2.80 (m, 1,5H), 3.08-3.23 (m, 1H), 3.59-4.04 (m, 1,5H), 4.14-4.52 (m, 1,5H), 4.89-4.96 (m, 0,5H), 5.29-5.68 (m, 1H), 5.72-5.81 (m, 2H), 7.13-7.28 (m, 3H), 7.32-7.39 (m, 2H), 7.84-8.07 (m, 1H), 10.28-10.61 (m, 1H). LCMS(ESI): [M]⁺ m/z: calcd 467.2; found 468.2; Rt=2.391 min.

Compound 86: Retention time: 24.36 min

¹H NMR (600 MHz, DMSO-d₆) δ (ppm) 0.36-0.49 (m, 2H), 0.71-0.77 (m, 1H), 0.78-0.94 (m, 6H), 0.99 (dd, 2H), 1.09 (d, 1H), 1.21-1.27 (m, 2H), 1.58-1.69 (m, 1H), 2.62-2.82 (m, 1H), 3.07-3.23 (m, 1H), 3.59-3.78 (m, 1H), 4.92 (d, 2H), 5.30-5.70 (m, 1H), 5.72-5.82 (m, 2H), 7.14-7.23 (m, 2H), 7.24-7.40 (m, 3H), 7.84-8.07 (m, 1H), 10.28-10.61 (m, 1H). LCMS(ESI): [M]⁺ m/z: calcd 467.2; found 468.2; Rt=2.397 min.

### Example 18. Compound 27 and Compound 64 N-(6-amino-5-methylpyridin-3-yl)-2-(2-(4-fluorophenyl)-5-methyl-4-(1-(trifluoromethyl)cyclopropyl)piperazin-1-yl)-2-oxoacetamide

Prepared as described in Scheme 1.1.

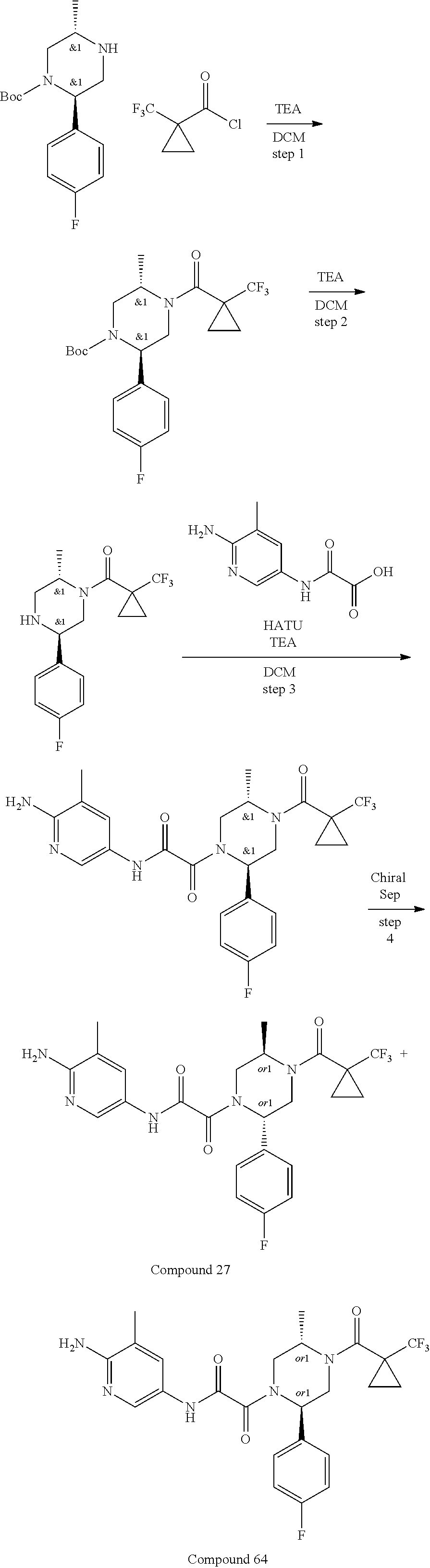

### Step 1: Synthesis of rac-(2R,5S)-tert-butyl 2-(4-fluorophenyl)-5-methyl-4-(1-(trifluoromethyl)cyclopropyl)piperazine-1-carboxylate

Prepared according to Scheme 1.1, step 1.1.8A. Yield: 0.37 g. LCMS(ESI): [M-Boc]⁺ m/z: calcd 330.2; found 331.2; Rt=1.398 min.

### Step 2: Synthesis of rac-(2R,5S)-5-(4-fluorophenyl)-2-methyl-1-(1-(trifluoromethyl)cyclopropyl)piperazine

Prepared according to Scheme 1.1, step 1.1.9. Yield: 0.27 g. LCMS(ESI): [M]⁺ m/z: calcd 330.2; found 331.2; Rt=0.947 min.

### Step 3: Synthesis of N-(6-amino-5-methylpyridin-3-yl)-2-(2-(4-fluorophenyl)-5-methyl-4-(1-(trifluoromethyl)cyclopropyl)piperazin-1-yl)-2-oxoacetamide

Prepared according to Scheme 1.1, step 1.1.10A. Yield: 95 mg (22.18%).

HPLC conditions: Column:SunFire 100*19 mm, 5 microM; 2-10 min 0-80% water-MeCN 30 ml/min (loading pump 4 ml MeCN). LCMS(ESI): [M]⁺ m/z: calcd 507.2; found 508.2; Rt=1.002 min.

### Step 4: Chiral Separation

Racemic N-(6-amino-5-methyl-3-pyridyl)-2-[(2S,5R)-2-(4-fluorophenyl)-5-methyl-4-[1-(trifluoromethyl)cyclopropyl]piperazin-1-yl]-2-oxo-acetamide (0.095 g, 198.14 umol) was chirally separated (Column: Chiralpak IA (250 * 30 mm, 5mkm); Mobile phase: Hexane-IPA-MeOH 50-25-25 Flow Rate:25 mL/min) to obtain N-(6-amino-5-methyl-3-pyridyl)-2-[(2S,5R)-2-(4-fluorophenyl)-5-methyl-4-[1-(trifluoromethyl)cyclopropyl]piperazin-1-yl]-2-oxo-acetamide (0.02861 g, 59.67 umol, 30.12% yield) (RT=27.06 min) and N-(6-amino-5-methyl-3-pyridyl)-2-[(2R,5S)-2-(4-fluorophenyl)-5-methyl-4-[1-(trifluoromethyl)cyclopropyl]piperazin-1-yl]-2-oxo-acetamide (0.02837 g, 59.17 umol, 29.86% yield) (RT=86.17 min).

Rel Time for Compound 27 in analytical conditions (column: IB, Hexane-IPA-MeOH, 70-15-15, 0.6 ml/min as mobile phase) 17.03 min and for Compound 64 18.65 min.

Compound 27: Retention time: 17.03 min

¹H NMR (600 MHz, DMSO-d₆) δ (ppm) 0.98-1.28 (m, 7H), 2.11 (s, 3H), 3.71 (m, 1H), 4.10 (m, 1H), 4.43-4.50 (m, 2H), 4.92 (m, 1H), 5.40 (m, 1H), 5.75 (m, 1H), 6.77 (m, 1H), 7.16-7.35 (m, 4H), 7.69 (s, 1H), 8.15 (s, 1H), 10.89 (m, 1H).

LCMS(ESI): [M]⁺ m/z: calcd 507.2; found 508.2; Rt=2.698 min.

Compound 64: Retention time: 18.65 min 1H NMR (600 MHz, DMSO-d₆) δ (ppm) 0.99 (m, 1H), 1.17-1.24 (m, 2H), 1.28 (m, 3H), 2.09 (s, 3H), 3.07 (m, 1H), 3.71 (m, 1H), 4.10 (m, 1H), 4.42-4.50 (m, 1H), 4.94 (m, 1H), 5.40 (s, 2H), 5.77 (m, 2H), 7.14-7.25 (m, 2H), 7.64-7.70 (m, 2H), 8.12-8.15 (m, 2H), 10.83 (m, 1H). LCMS(ESI): [M]⁺ m/z: calcd 507.2; found 508.2; Rt=2.696 min.

### The synthesis of N-(6-amino-5-methylpyridin-3-yl)-2-((2S,5R)-2-(4-fluorophenyl)-5-methyl-4-(1-(trifluoromethyl)cyclopropanecarbonyl)piperazin-1-yl)-2-oxoacetamide

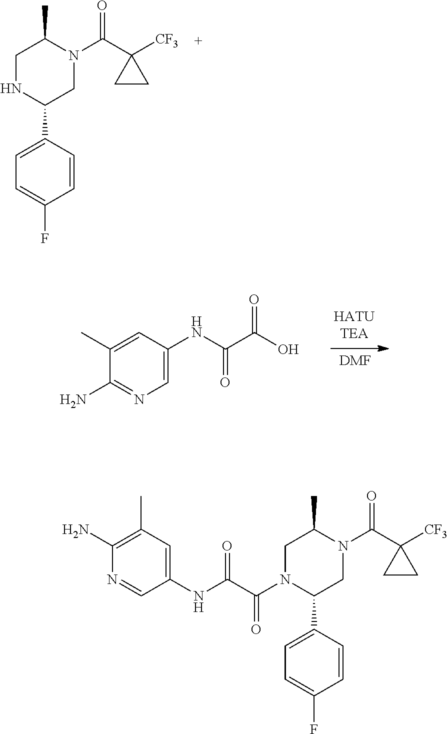

Prepared according to Scheme 1.1, step 1.4.9A. Yield: 70 mg (27.12%).

HPLC conditions: Column: YMC Triart C18 100*20 mm, 5 microM; 0-5 min 40-55% MeOH/water+0.1% NH₄OH 40 ml/min; (loading pump 4 ml/min MeOH).

### Compound 27 Lot 2 (Chiral Synthesis):

¹H NMR (600 MHz, DMSO-d₆) δ (ppm) 0.94-1.02 (m, 1H), 1.13-1.25 (m, 3H), 1.26-1.36 (m, 3H), 1.97-2.04 (m, 3H), 2.80-3.23 (m, 1H), 3.31-3.50 (m, 1H), 3.56-4.05 (m, 1H), 4.06-4.44 (m, 1H), 4.44-5.06 (m, 1H), 5.30-5.62 (m, 1H), 5.62-5.82 (m, 2H), 7.12-7.20 (m, 2H), 7.22-7.32 (m, 1H), 7.32-7.41 (m, 1H), 7.42-7.59 (m, 1H), 7.96-8.08 (m, 1H), 10.40-10.78 (m, 1H). LCMS(ESI): [M]⁺ m/z: calcd 507.2; found 508.2; Rt=2.526 min.

### Example 19. Compound 158 and Compound 23 N-(6-amino-5-methylpyridin-3-yl)-2-((2S,5R)-2-(3,4-difluorophenyl)-4-isobutyryl-5-methylpiperazin-1-yl)-2-oxoacetamide

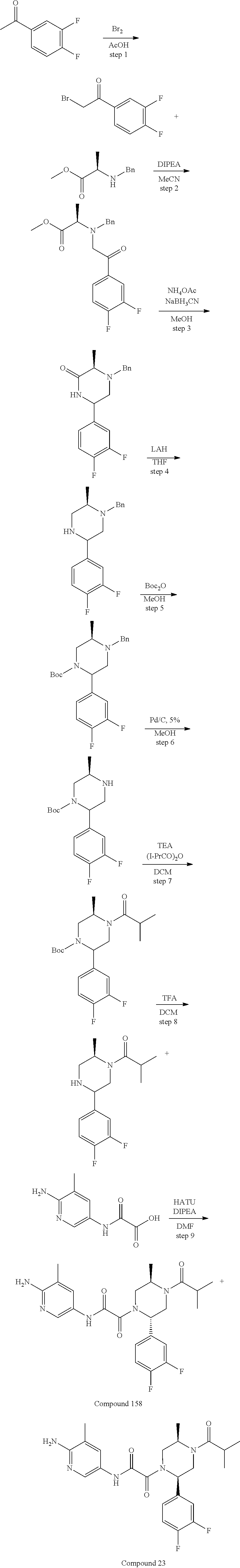

### Step 1: Synthesis of 2-bromo-1-(3,4-difluorophenyl)ethanone

Prepared according to 0, step 1.1.3A. Yield: 12 g (99.65%).

GCMS: calcd 235.2; found 235.2; Rt=5.575 min.

### Step 2: Synthesis of (R)-methyl 2-(benzyl(2-(3,4-difluorophenyl)-2-oxoethyl)amino)propanoate

To a stirred solution of DIPEA (3.93 g, 30.43 mmol, 5.30 mL) and methyl (2R)-2-(benzylamino)propanoate (4.9 g, 25.36 mmol) in MeCN (50 mL) were added a solution of 2-bromo-1-(3,4-difluorophenyl)ethanone (8.19 g, 27.89 mmol) in MeCN (50 mL) during 5 min. The resulting reaction mixture was stirred at 25° C. for 16 hr. Then reaction mixture was evaporated dryness and was quenched with EtOAc 100 ml. The organic phase was washed by water (3*20 ml), dried at Na₂SO₄ and concentrated under reduced pressure to obtain crude product methyl (2R)-2-[benzyl-[2-(3,4-difluorophenyl)-2-oxo-ethyl]amino]propanoate (8.5 g, 24.47 mmol, 96.51% yield) as red oil.

LCMS(ESI): [M]⁺ m/z: calcd 347.2; found 348.2; Rt=1.388 min.

### Step 3: Synthesis of (3R)-4-benzyl-6-(3,4-difluorophenyl)-3-methylpiperazin-2-one

Prepared according to Scheme 1.1, step 1.1.4. Yield: 7 g (49.78%).

LCMS(ESI): [M]⁺ m/z: calcd 316.2; found 317.2; Rt=1.110 min.

### Step 4: Synthesis of (2R)-1-benzyl-5-(3, 4-difluorophenyl)-2-methylpiperazine

Prepared according to Scheme 1.1, step 1.1.5. Yield: 6.5 g (97.15%).

LCMS(ESI): [M]⁺ m/z: calcd 302.2; found 303.2; Rt=1.048 min.

### Step 5: Synthesis of (5R)-tert-butyl 4-benzyl-2-(3,4-difluorophenyl)-5-methylpiperazine-1-carboxylate

(2R)-1-Benzyl-5-(3,4-difluorophenyl)-2-methyl-piperazine (6.5 g, 21.50 mmol) was dissolved in MeOH (70 mL) and Boc₂O (4.69 g, 21.50 mmol, 4.93 mL) was added dropwise to the resulting mixture. The reaction mixture was stirred during 16 hr at 25° C. The reaction mixture was concentrated in vacuum. The residue was diluted with DCM (50 ml) and organic layer were dried over Na₂SO₄, filtered and evaporated, crude product tert-butyl (2S,5R)-4-benzyl-2-(3,4-difluorophenyl)-5-methyl-piperazine-1-carboxylate (0.242 g, 601.28 umol, 2.80% yield) as yellow oil was obtained. 1.2 g of Product was purified by reverse phase HPLC to give 0.242 g. of tert-butyl (2S,5R)-4-benzyl-2-(3,4-difluorophenyl)-5-methyl-piperazine-1-carboxylate (0.242 g, 601.28 umol, 2.80% yield).

LCMS(ESI): [M]⁺ m/z: calcd 402.2; found 403.2; Rt=3.181 min.

### Step 6: Synthesis of (5R)-tert-butyl 2-(3,4-difluorophenyl)-5-methylpiperazine-1-carboxylate

Prepared according to Scheme 1.1, step 1.1.7. Yield: 3.4 g (87.62%).

LCMS(ESI): [M]⁺ m/z: calcd 312.2; found 313.2; Rt=0.951 min.

### Step 7: Synthesis of (5R)-tert-butyl 2-(3,4-difluorophenyl)-4-isobutyryl-5-methylpiperazine-1-carboxylate

Prepared according to Scheme 1.1, step 1.1.8D. Yield: 0.95 g (96.99%).

LCMS(ESI): [M-t-Bu]⁺ m/z: calcd 326.2; found 327.2; Rt=1.505 min.

### Step 8: Synthesis of 1-((2R)-5-(3,4-difluorophenyl)-2-methylpiperazin-1-yl)-2-methylpropan-1-one

Prepared according to Scheme 1.1, step 1.1.9. Yield: 0.55 g (78.42%).

LCMS(ESI): [M]⁺ m/z: calcd 282.2; found 283.2; Rt=0.943 min.

### Step 9: Synthesis of N-(6-amino-5-methylpyridin-3-yl)-2-((2S,5R)-2-(3,4-difluorophenyl)-4-isobutyryl-5-methylpiperazin-1-yl)-2-oxoacetamide

Prepared according to Scheme 1.1, step 1.1.10B. Yield: 12.51 mg (2.56% for Compound 158) and 26.76 mg (5.48% for Compound 23).

HPLC conditions: Column: YMC Triart C18 100*20 mm, 5 microM; 0-5 min 30-80% MeOH/water+0.1% NH₄OH 30 ml/min; (loading pump 4 ml/min MeOH).

Compound 158: ¹H NMR (600 MHz, DMSO-d₆) δ (ppm) 0.72-0.87 (m, 3H), 0.91-1.00 (m, 3H), 1.07-1.26 (m, 3H), 2.00-2.07 (m, 3H), 2.64-3.24 (m, 2H), 3.61-4.51 (m, 3H), 4.85-5.68 (m, 4H), 7.06-7.28 (m, 2H), 7.34-7.46 (m, 2H), 7.48-7.53 (m, 1H), 10.49-10.66 (m, 1H). LCMS(ESI): [M]⁺ m/z: calcd 459.2; found 460.2; Rt=2.647 min.

Compound 23: ¹H NMR (600 MHz, DMSO-d₆) δ (ppm) 0.96-1.18 (m, 9H), 1.93-2.03 (m, 3H), 2.68-3.23 (m, 2H), 3.41-3.48 (m, 1H), 4.11-5.33 (m, 4H), 5.56-5.66 (m, 2H), 7.14-7.30 (m, 1H), 7.31-7.61 (m, 3H), 7.69-8.07 (m, 1H), 10.15-10.56 (m, 1H).

LCMS(ESI): [M]⁺ m/z: calcd 459.2; found 460.2; Rt=2.765 min.

### Example 20. Compound 106 N-(6-amino-5-methylpyridin-3-yl)-2-(2-(4-fluorophenyl)-5-methyl-4-pivaloylpiperazin-1-yl)-2-oxoacetamide

Prepared as described in Scheme 1.1.

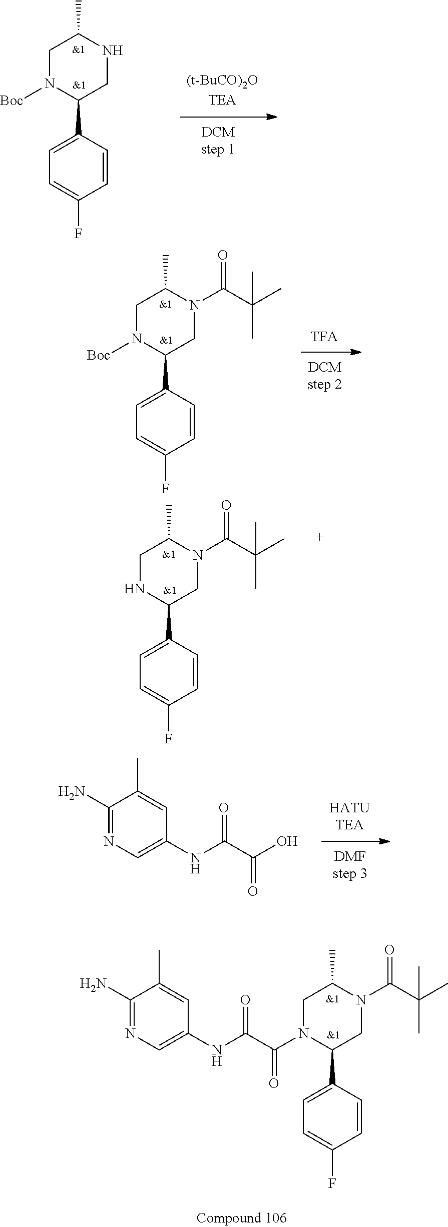

### Step 1: Synthesis of rac-(2R,5S)-tert-butyl 2-(4-fluorophenyl)-5-methyl-4-pivaloylpiperazine-1-carboxylate

Prepared according to Scheme 1.1, step 1.1.8A. Yield: 0.2 g of crude.

LCMS(ESI): [M-Boc]⁺ m/z: calcd 278.2; found 279.2; Rt=1.172 min.

### Step 2: Synthesis of rac-1-((2R,5S)-5-(4-fluorophenyl)-2-methylpiperazin-1-yl)-2,2-dimethylpropan-1-one

Prepared according to Scheme 1.1, step 1.1.9. Yield: 0.1 g of crude.

LCMS(ESI): [M]⁺ m/z: calcd 278.2; found 279.2; Rt=0,796 min.

### Step 3: Synthesis of rac-N-(6-amino-5-methylpyridin-3-yl)-2-(2-(4-fluorophenyl)-5-methyl-4-pivaloylpiperazin-1-yl)-2-oxoacetamide

Prepared according to Scheme 1.1, step 1.1.10A. Yield: 14 mg (8.56%).

Compound 106: ¹H NMR (600 MHz, DMSO-d₆) δ (ppm) 1.02-1.08 (m, 9H), 1.23-1.24 (m, 3H), 2.00-2.03 (m, 3H), 3.62 (d, 1H), 4.02 (d, 1H), 4.43-4.47 (m, 2H), 4.84 (m, 1H), 5.26 (m, 1H), 5.60-5.64 (m, 2H), 7.15-7.20 (m, 2H), 7.28-7.30 (m, 1H), 7.36-7.38 (m, 1H), 7.48 (s, 1H), 7.98-8.00 (m, 1H), 10.57 (m, 1H). LCMS(ESI): [M]⁺ m/z: calcd 455.2; found 456.2; Rt=0.851 min.

### Example 21. Compound 108 N-(6-amino-5-methylpyridin-3-yl)-2-(2-(4-fluorophenyl)-5-methyl-4-(1-methylcyclopropanecarbonyl)piperazin-1-yl)-2-oxoacetamide

Prepared as described in Scheme 1.1,

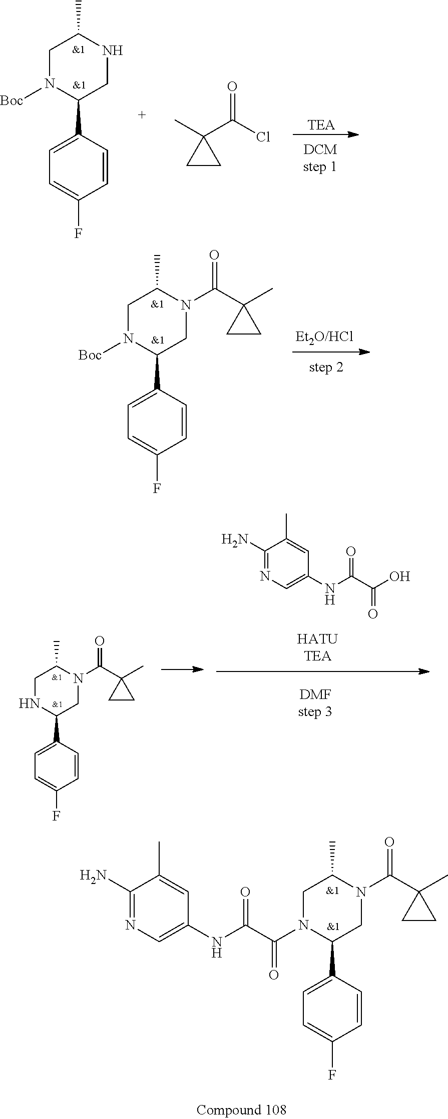

Step 1: Synthesis of rac-(2R,5S)-tert-butyl 2-(4-fluorophenyl)-5-methyl-4-(1-methylcyclopropanecarbonyl)piperazine-1-carboxylate

Prepared according to Scheme 1.1, step 1.1.8A. Yield: 320 mg of crude. LCMS(ESI): [M-Boc]⁺ m/z: calcd 276.2; found 277.2; Rt=1.482 min.

### Step 2: Synthesis of rac-((2R,5S)-5-(4-fluorophenyl)-2-methylpiperazin-1-yl)(1-methylcyclopropyl)methanone

Prepared as described in Scheme 1.1, method B. Yield: 315 mg (HCl; crude).

LCMS(ESI): [M]⁺ m/z: calcd 276.2; found 277.2; Rt=0.707 min.

### Step 3: Synthesis of N-(6-amino-5-methylpyridin-3-yl)-2-(2-(4-fluorophenyl)-5-methyl-4-(1-methylcyclopropanecarbonyl)piperazin-1-yl)-2-oxoacetamide

Prepared according to Scheme 1.1, step 1.1.10A. Yield: 28.7 mg (6.28%).

HPLC conditions: Column:SunFire 100*19 mm, 5 microM; 15-35 min 40-75% MeCN 30 ml/min (loading pump 4 ml MeCN).

Compound 108: ¹H NMR (600 MHz, DMSO-d₆) δ (ppm) 0.64-0.81 (m, 4H), 1.05 (s, 3H), 1.25 (m, 3H), 2.03 (m, 3H), 3.22 (m, 1H), 3.66 (m, 1H), 4.07 (m, 1H), 4.46-4.53 (m, 2H), 4.86 (m, 1H), 5.30 (m, 1H), 5.63 (m, 2H), 7.15-7.21 (m, 2H), 7.28-7.38 (m, 2H), 7.48 (s, 1H), 8.02 (s, 1H). LCMS(ESI): [M]⁺ m/z: calcd 453.2; found 454.2; Rt=0.867 min.

### Example 22. Compound 148 tert-butyl 4-(2-((6-amino-5-cyclopropylpyridin-3-yl)amino)-2-oxoacetyl)-5-(4-fluorophenyl)-2-methylpiperazine-1-carboxylate

Prepared as described in Scheme 1.1.

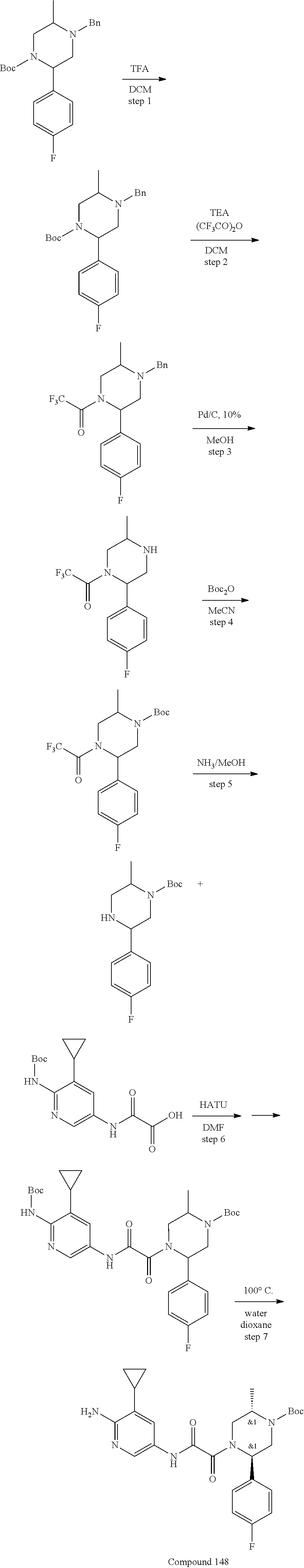

### Step 1: Synthesis of 1-benzyl-5-(4-fluorophenyl)-2-methylpiperazine

tert-butyl 4-benzyl-2-(4-fluorophenyl)-5-methyl-piperazine-1-carboxylate (3.04 g, 7.90 mmol) was dissolved in DCM (10 mL) and TFA (10 mL) was added thereto. The resulting mixture was stirred for 1 hr. The reaction mixture was carefully poured into K₂CO₃ solution (25 g in 100 ml of water) and the resulting mixture was extracted with DCM (2*40 ml). Combined organic layers were dried over Na₂SO₄, filtered and concentrated in vacuo to obtain 1-benzyl-5-(4-fluorophenyl)-2-methyl-piperazine (2.23 g, 7.84 mmol, 99.28% yield). LCMS(ESI): [M]⁺ m/z: calcd 284.2; found 285.2; Rt=0.813 min.

### Step 2: Synthesis of 4-benzyl-2-(4-fluorophenyl)-5-methylpiperazin-1-yl)-2,2,2-trifluoroethanone

1-benzyl-5-(4-fluorophenyl)-2-methyl-piperazine (2.25 g, 7.91 mmol) was dissolved in DCM (50 mL) and Triethylamine (2.40 g, 23.74 mmol, 3.31 mL) was added thereto. The resulting mixture was cooled to −5° C. in an ice/methanol bath and (2,2,2-trifluoroacetyl) 2,2,2-trifluoroacetate (2.16 g, 10.29 mmol, 1.45 mL) was added dropwise to the reaction mixture. After addition completed, the reaction mixture was allowed to warm to room temperature and stirred overnight. Water (40 ml) was added to the reaction mixture and an organic layer was separated. The aqueous layer was extracted with DCM (50 ml) and combined organic layers were dried over Na₂SO₄, filtered and concentrated in vacuo. The residue was purified by column chromatography to obtain 1-[4-benzyl-2-(4-fluorophenyl)-5-methyl-piperazin-1-yl]-2,2,2-trifluoro-ethanone (1.95 g, 5.13 mmol, 64.79% yield).

LCMS(ESI): [M]⁺ m/z: calcd 380.2; found 381.2; Rt=1.468 min.

### Step 3: Synthesis of 2,2,2-trifluoro-1-(2-(4-fluorophenyl)-5-methylpiperazin-1-yl)ethanone

1-[4-benzyl-2-(4-fluorophenyl)-5-methyl-piperazin-1-yl]-2,2,2-trifluoro-ethanone (1.95 g, 5.13 mmol) was dissolved in MeOH (50 mL) and Palladium, 10% on carbon, Type 487, dry (436.45 mg, 4.10 mmol) was added thereto. The reaction vessel was evacuated and backfilled three times with hydrogen and the resulting mixture was hydrogenated at 1 atm (balloon) at room temperature overnight. The catalyst was filtered off and the filtrate was concentrated in vacuo to obtain 2,2,2-trifluoro-1-[2-(4-fluorophenyl)-5-methyl-piperazin-1-yl]ethanone (1.41 g, 4.85 mmol, 94.56% yield). LCMS(ESI): [M]⁺ m/z: calcd 290.2; found 291.2; Rt=0.724 min.

### Step 4: Synthesis of tert-butyl 5-(4-fluorophenyl)-2-methyl-4-(2,2,2-trifluoroacetyl)piperazine-1-carboxylate

2,2,2-Trifluoro-1-[2-(4-fluorophenyl)-5-methyl-piperazin-1-yl]ethanone (0.703 g, 2.42 mmol) was dissolved in MeCN (7 mL) and di-tert-butyl dicarbonate (528.59 mg, 2.42 mmol, 555.83 uL) was added thereto. The resulting mixture was stirred overnight. The reaction mixture was concentrated in vacuum to obtain tert-butyl 5-(4-fluorophenyl)-2-methyl-4-(2,2,2-trifluoroacetyl)piperazine-1-carboxylate (0.954 g, crude). LCMS(ESI): [M-Boc]⁺ m/z: calcd 290.2; found 291.2; Rt=1.478 min.

### Step 5: Synthesis of tert-butyl 5-(4-fluorophenyl)-2-methylpiperazine-1-carboxylate

tert-Butyl 5-(4-fluorophenyl)-2-methyl-4-(2,2,2-trifluoroacetyl)piperazine-1-carboxylate (954 mg, 2.44 mmol) was dissolved in NH₃/MeOH (15 mL) and the resulting mixture was stirred overnight. The reaction mixture was concentrated in vacuum. Conversion rate was 56% by LCMS. The residue was dissolved in NH₃/MeOH (25 mL) and the resulting mixture was allowed to stir overnight. The reaction mixture was concentrated in vacuum. Conversion rate was 86% by LCMS. The residue was dissolved in NH₃/MeOH (25 mL) and the resulting mixture was allowed to stir overnight. The reaction mixture was concentrated in vacuum to obtain tert-butyl 5-(4-fluorophenyl)-2-methyl-piperazine-1-carboxylate (706 mg, 2.40 mmol, 98.14% yield). LCMS(ESI): [M]⁺ m/z: calcd 294.2; found 295.2; Rt=1.013 min.

### Step 6: Synthesis of tert-butyl 4-(2-((6-((tert-butoxycarbonyl)amino)-5-cyclopropylpyridin-3-yl)amino)-2-oxoacetyl)-5-(4-fluorophenyl)-2-methylpiperazine-1-carboxylate

Prepared as described in Scheme 1.1, step 1.1.10A. Yield: 277.7 mg (45.59%).

HPLC conditions: Column:SunFire 100*19 mm, 5 microM; 2-10 min 60-75% MeOH/water 30 ml/min; (loading pump 4 ml/min MeOH).

LCMS(ESI): [M]⁺ m/z: calcd 597.2; found 598.2; Rt=1.378 min.

### Step 7: Synthesis of tert-butyl 4-(2-((6-amino-5-cyclopropylpyridin-3-yl)amino)-2-oxoacetyl)-5-(4-fluorophenyl)-2-methylpiperazine-1-carboxylate

Prepared by Boc Group deprotection method A. Yield: 43.6 mg (18.86%).

¹H NMR (500 MHz, DMSO-d₆) δ (ppm) 0.38-0.48 (m, 2H), 0.82-0.91 (m, 2H), 1.09-1.17 (m, 3H), 1.30-1.38 (m, 9H), 1.59-1.70 (m, 1H), 2.73-3.07 (m, 1H), 3.36-3.52 (m, 1H), 3.54-3.96 (m, 1H), 4.02-4.22 (m, 1H), 4.30-4.43 (m, 1H), 5.20-5.56 (m, 1H), 5.72-5.80 (m, 2H), 7.16-7.25 (m, 2H), 7.26-7.41 (m, 3H), 7.91-8.07 (m, 1H), 10.32-10.63 (m, 1H). LCMS(ESI): [M]⁺ m/z: calcd 497.2; found 498.2; Rt=2.638 min.

### Example 23. Compound 75 N-(6-amino-5-cyclopropylpyridin-3-yl)-2-((SR)-2-(3,4-difluorophenyl)-4-isobutyryl-5-methylpiperazin-1-yl)-2-oxoacetamide

Prepared as described in Scheme 1.1.

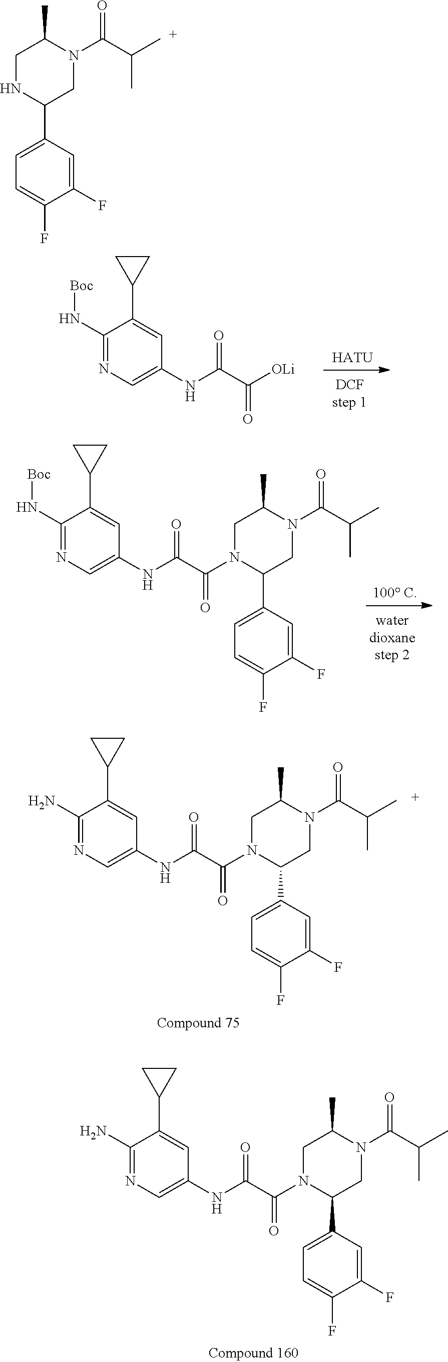

### Step 1: Synthesis of tert-butyl (3-cyclopropyl-5-(2-((2S,5R)-2-(3,4-difluorophenyl)-4-isobutyryl-5-methylpiperazin-1-yl)-2-oxoacetamido)pyridin-2-yl)carbamate

Prepared as described in Scheme 1.1, step 1.1.10A. Yield: 0.7 g of crude.

LCMS(ESI): [M]⁺ m/z: calcd 585.2; found 586.2; Rt=1.285 min.

### Step 2: Synthesis of N-(6-amino-5-cyclopropylpyridin-3-yl)-2-((5R)-2-(3,4-difluorophenyl)-4-isobutyryl-5-methylpiperazin-1-yl)-2-oxoacetamide

Prepared by Boc Group eprotection method A. Yield: 31 mg (5.34% for Compound 75) and 62.1 mg (10.70% for Compound 160). HPLC conditions: Column: YMC Triart C18 100*20 mm, 5 microM; 0-5 min 30-80% MeOH/water+0.1% NH₄OH 30 ml/min; (loading pump 4 ml/min MeOH).

Compound 75: ¹H NMR (600 MHz, DMSO-d₆) δ (ppm) 0.37-0.48 (m, 2H), 0.71-0.89 (m, 5H), 0.91-1.03 (m, 3H), 1.09-1.27 (m, 3H), 1.60-1.70 (m, 1H), 2.65-2.84 (m, 1H), 3.07-3.26 (m, 2H), 3.38-3.77 (m, 1H), 3.77-4.19 (m, 1H), 4.20-4.93 (m, 1H), 5.29-5.68 (m, 1H), 5.72-5.86 (m, 2H), 7.04-7.35 (m, 2H), 7.35-7.47 (m, 2H), 7.82-8.09 (m, 1H), 10.26-10.69 (m, 1H). LCMS(ESI): [M]⁺ m/z: calcd 485.2; found 486.2; Rt=2.234 min.

Compound 160: ¹H NMR (600 MHz, DMSO-d₆) δ (ppm) 0.33-0.50 (m, 2H), 0.78-0.90 (m, 2H), 0.96-1.21 (m, 9H), 1.58-1.71 (m, 1H), 2.72-3.06 (m, 1H), 3.33-3.50 (m, 2H), 3.96-4.29 (m, 2H), 4.30-4.69 (m, 1H), 4.87-5.36 (m, 1H), 5.68-5.81 (m, 2H), 6.93-7.28 (m, 1H), 7.29-7.37 (m, 1H), 7.37-7.45 (m, 1H), 7.45-7.60 (m, 1H), 7.72-8.11 (m, 1H), 9.95-10.64 (m, 1H). LCMS(ESI): [M]⁺ m/z: calcd 485.2; found 486.2; Rt=2.471 min.

### Example 24. Compound 22 1-(6-amino-5-cyclopropylpyridin-3-yl)-2-((2S,5R)-2-(3,4-difluorophenyl)-5-methyl-4-(1-(trifluoromethyl)cyclopropanecarbonyl)piperazin-1-yl)ethane-1,2-dione (Compound 22)

Prepared as described in Scheme 1.1.

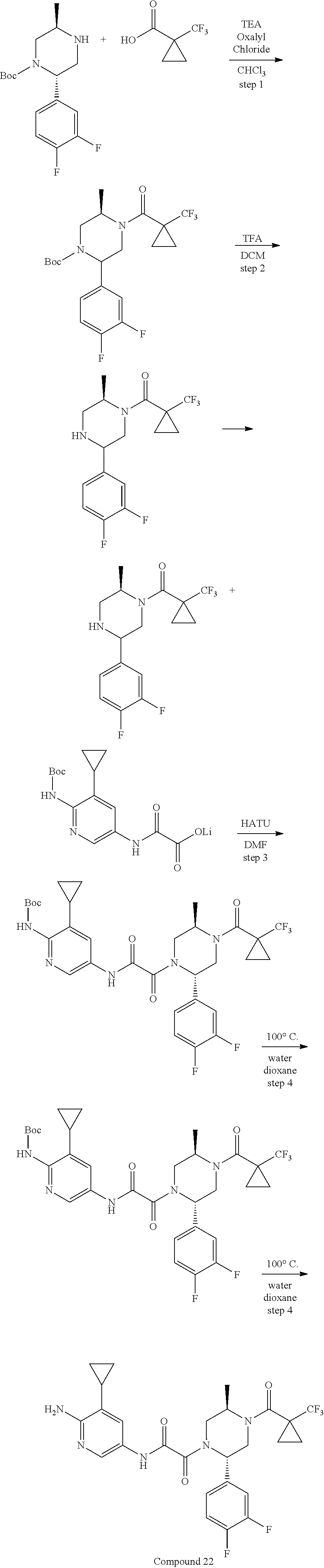

### Step 1: Synthesis of (5R)-tert-butyl 2-(3,4-difluorophenyl)-5-methyl-4-(1-(trifluoromethyl)cyclopropanecarbonyl)piperazine-1-carboxylate

Prepared as described in Scheme 1.1, step 1.1.8E. Yield: 0.2 g (75.30%).

LCMS(ESI): [M-t-Bu]⁺ m/z: calcd 392.2; found 393.2; Rt=1.406 min.

### Step 2: Synthesis of ((2R)-5-(3,4-difluorophenyl)-2-methylpiperazin-1-yl)(1-(trifluoromethyl)cyclopropyl)methanone

Prepared as described in Scheme 1.1, step 1.1.9. Yield: 0.13 g (83.68%).

LCMS(ESI): [M]⁺ m/z: calcd 348.2; found 349.2; Rt=0.989 min.

### Step 3: Synthesis of tert-butyl (3-cyclopropyl-5-(2-((2S,5R)-2-(3,4-difluorophenyl)-5-methyl-4-(1-(trifluoromethyl)cyclopropanecarbonyl)piperazin-1-yl)-2-oxoacetamido)pyridin-2-yl)carbamate

Prepared as described in 0, step 1.1.10A. Yield: 0.15 g of crude. LCMS(ESI): [M]⁺ m/z: calcd 651.2; found 652.2; Rt=1.496 min.

### Step 4: Synthesis of 1-(6-amino-5-cyclopropylpyridin-3-yl)-2-((2S,5R)-2-(3,4-difluorophenyl)-5-methyl-4-(1-(trifluoromethyl)cyclopropanecarbonyl)piperazin-1-yl)ethane-1,2-dione

Prepared by Boc-deprotection method A. Yield: 70 mg (43.72%). HPLC conditions: Column: YMC Triart C18 100*20 mm, 5 microM; 0-5 min 45-78% MeOH/water+0.1% NH₄OH 30 ml/min; (loading pump 4 ml/min MeOH).

Compound 22: ¹H NMR (600 MHz, DMSO-d₆) δ (ppm) 0.43-0.48 (m, 2H), 0.86-0.90 (m, 2H), 0.97-1.04 (m, 1H), 1.13-1.20 (m, 2H), 1.24-1.33 (m, 4H), 1.59-1.68 (m, 1H), 2.59-3.09 (m, 1H), 3.34-3.84 (m, 1H), 3.89-4.18 (m, 1H), 4.24-4.97 (m, 2H), 5.39-5.76 (m, 1H), 5.77-5.84 (m, 2H), 7.10-7.26 (m, 1H), 7.32-7.35 (m, 1H), 7.35-7.48 (m, 2H), 7.98-8.17 (m, 1H), 10.35-10.83 (m, 1H). LCMS(ESI): [M]⁺ m/z: calcd 551.2; found 552.2; Rt=2.813 min.

### Example 25. Compound 51 N-(6-amino-5-cyclopropylpyridin-3-yl)-2-((2S,5R)-5-methyl-2-phenyl-4-(1-(trifluoromethyl)cyclopropanecarbonyl)piperazin-1-yl)-2-oxoacetamide

Prepared as described in Scheme 1.1.

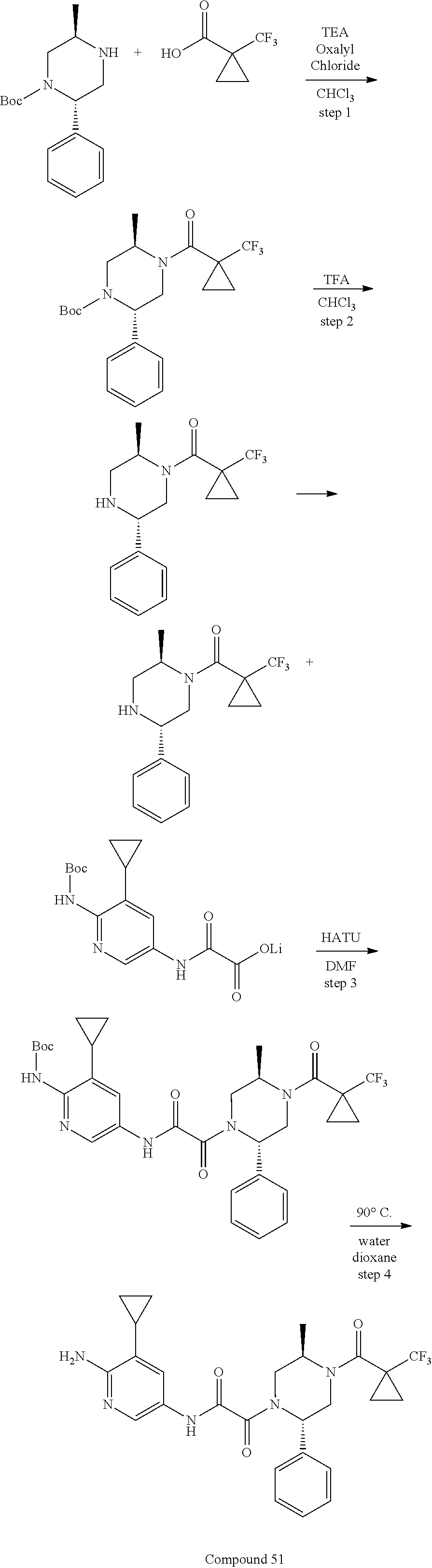

### Step 1: Synthesis of (2S,5R)-tert-butyl 5-methyl-2-phenyl-4-(1-(trifluoromethyl)cyclopropanecarbonyl)piperazine-1-carboxylate

1-(Trifluoromethyl)cyclopropanecarboxylic acid (0.2 g, 1.30 mmol) and few drops of DMF were mixed together in CHCl₃ (6 mL) and the resulting solution was cooled to 5° C. in an ice bath. Oxalyl chloride (192.89 mg, 1.52 mmol, 132.11 uL) was added dropwise to the previous solution and the resulting mixture was allowed to warm to rt and stirred for 2 hr. After completion the reaction mixture was poured into aq·K₂CO₃ solution (4 g in 10 ml of water) and the resulting mixture was extracted with CHCl₃ (2*10 ml). Combined organic layers were dried over Na₂SO₄ and added dropwise at 0° C. to the stirred solution of tert-butyl (2S,5R)-5-methyl-2-phenyl-piperazine-1-carboxylate (0.28 g, 1.01 mmol) and TEA (153.78 mg, 1.52 mmol, 211.81 uL) in CHCl₃ (6 mL).The resulting mixture was allowed to warm to rt and stirred overnight. After completion the reaction mixture was diluted with CHCl₃ (5 ml) and the resulting solution was washed with water (2*10 ml), dried over Na₂SO₄, filtered and evaporated to afford tert-butyl (2S,5R)-5-methyl-2-phenyl-4-[1-(trifluoromethyl)cyclopropanecarbonyl]piperazine-1-carboxylate (0.4 g, 969.83 umol, 95.73% yield). LCMS(ESI): [M-Boc]⁺ m/z: calcd 312.2; found 313.2; Rt=1.456 min.

### Step 2: Synthesis of ((2R,5S)-2-methyl-5-phenylpiperazin-1-yl)(1-(trifluoromethyl)cyclopropyl)methanone

Prepared as described in 0, step 1.1.9. Yield: 0.3 g (99.04%). LCMS(ESI): [M]⁺ m/z: calcd 312.2; found 313.2; Rt=0.922 min.

### Step 3: Synthesis of tert-butyl (3-cyclopropyl-5-(2-((2S,5R)-5-methyl-2-phenyl-4-(1-(trifluoromethyl)cyclopropanecarbonyl)piperazin-1-yl)-2-oxoacetamido)pyridin-2-yl)carbamate

Prepared as described in Scheme 1.1, step 1.1.10A. Yield: 0.29 g of crude.

LCMS(ESI): [M]⁺ m/z: calcd 615.2; found 616.2; Rt=1.342 min.

### Step 4: Synthesis of N-(6-amino-5-cyclopropylpyridin-3-yl)-2-((2S,5R)-5-methyl-2-phenyl-4-(1-(trifluoromethyl)cyclopropanecarbonyl)piperazin-1-yl)-2-oxoacetamide

Prepared as described in 0, method A. Yield: 130 mg (53.53%). HPLC conditions: Column: YMC Triart C18 100*20 mm, 5 microM; 0-5 min 40-90% MeOH/water+0.1% NH₄OH 30 ml/min; (loading pump 4 ml/min MeOH).

Compound 51: ¹H NMR (600 MHz, DMSO-d₆) δ (ppm) 0.40-0.52 (m, 2H), 0.85-0.91 (m, 2H), 0.93-1.01 (m, 1H), 1.11-1.23 (m, 3H), 1.25-1.35 (m, 3H), 1.58-1.69 (m, 1H), 2.57-3.12 (m, 1H), 3.19-3.29 (m, 0.7H), 3.63-3.73 (m, 0.3H), 3.83-4.57 (m, 2H), 4.88-5.29 (m, 1H), 5.38-5.76 (m, 1H), 5.76-5.87 (m, 2H), 7.21-7.37 (m, 6H), 7.99-8.15 (m, 1H), 10.56 (br s, 1H). LCMS(ESI): [M]⁺ m/z: calcd 515.2; found 516.2; Rt=2.870 min.

### Example 26. Compound 122 N-(6-amino-5-cyclopropylpyridin-3-yl)-2-((2S,5R)-4-isobutyl-5-methyl-2-phenylpiperazin-1-yl)-2-oxoacetamide

Prepared as described in Scheme 1.1.

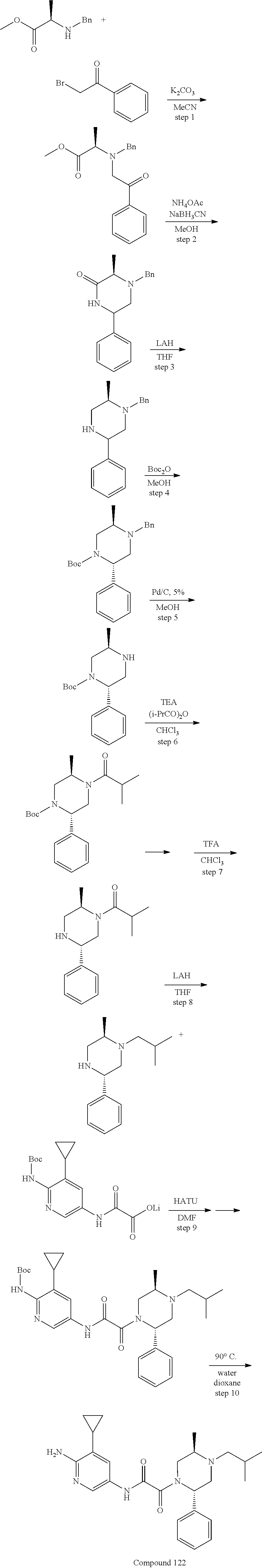

### Step 1: Synthesis of (R)-methyl 2-(benzyl(2-oxo-2-phenylethyl)amino)propanoate

Prepared as described in Scheme 1.1, step 1.1.3. Yield: 6.6 g of crude. LCMS(ESI): [M]⁺ m/z: calcd 311.2; found 312.2; Rt=1.439 min.

### Step 2: Synthesis of (3R)-4-benzyl-3-methyl-6-phenylpiperazin-2-one

Prepared as described in Scheme 1.1, step 1.1.4. Yield: 4.6 g (77.41%). CC conditions: The crude product was purified by silica gel with EtOAc/CHCl₃ (1:10) as an eluent mixture. LCMS(ESI): [M]⁺ m/z: calcd 280.2; found 281.2; Rt=1.123 min.

### Step 3: Synthesis of (2R)-1-benzyl-2-methyl-5-phenylpiperazine

Prepared as described in Scheme 1.1, step 1.1.5. Yield: 2.1 g (85.01%). LCMS(ESI): [M]⁺ m/z: calcd 266.2; found 267.2; Rt=0.959 min.

### Step 4: Synthesis of (2S,5R)-tert-butyl 4-benzyl-5-methyl-2-phenylpiperazine-1-carboxylate

(2R)-1-Benzyl-2-methyl-5-phenyl-piperazine (2.1 g, 7.88 mmol) was dissolved in MeOH (50 mL), tert-butoxycarbonyl tert-butyl carbonate (2.06 g, 9.46 mmol, 2.17 mL) was added dropwise to the resulting mixture. The reaction mixture was stirred overnight. After completion, the reaction mixture was concentrated in vacuum. The residue was diluted with water (20 ml) and the resulting mixture was extracted with CHCl₃ (2*20 ml). Combined organic layers were washed with brine (25 ml), dried over Na₂SO₄, filtered and evaporated. The residue was purified by reverse phase HPLC chromatography (Column: Chromatorex 18 SMB 100-5T 100*19 mm, 5 microM; 0-6 min 20-50% water-MeCN 30 ml/min (loading pump 4 ml MeCN)) to give tert-butyl (2S,5R)-4-benzyl-5-methyl-2-phenyl-piperazine-1-carboxylate (0.75 g, 2.05 mmol, 25.96% yield). LCMS(ESI): [M]⁺ m/z: calcd 366.2; found 367.2; Rt=3.202 min.

### Step 5: Synthesis of (2S,5R)-tert-butyl 5-methyl-2-phenylpiperazine-1-carboxylate

Prepared as described in Scheme 1.1, step 1.1.7. Yield: 0.39 g (97.58%).

LCMS(ESI): [M]⁺ m/z: calcd 276.2; found 277.2; Rt=0.997 min.

### Step 6: Synthesis of (2S,5R)-tert-butyl 4-isobutyryl-5-methyl-2-phenylpiperazine-1-carboxylate

Prepared as described in Scheme 1.1, step 1.1.8A. Yield: 0.2 g (93.85%).

LCMS(ESI): [M]⁺ m/z: calcd 346.2; found 347.2; Rt=1.559 min.

### Step 7: Synthesis of 2-methyl-1-((2R,5S)-2-methyl-5-phenylpiperazin-1-yl)propan-1-one

Prepared as described in Scheme 1.1, step 1.1.9. Yield: 0.14 g (98.45%).

LCMS(ESI): [M]⁺ m/z: calcd 246.2; found 247.2; Rt=0.821 min.

### Step 8: Synthesis of (2R,5S)-1-isobutyl-2-methyl-5-phenylpiperazine

LAH (64.71 mg, 1.70 mmol) was suspended in THF (5 mL) and the resulting suspension was heated to reflux. A solution of 2-methyl-1-[(2R,5S)-2-methyl-5-phenyl-piperazin-1-yl]propan-1-one (0.14 g, 568.30 umol) in THF (5 mL) was added dropwise to the previous suspension, maintaining a gentle reflux. After addition completed, the reaction mixture was refluxed for 16 hr and then allowed to cool to rt and stirred overnight. After completion, water (0.1 ml) was carefully added dropwise to the precooled reaction mixture followed by addition of a aq. KOH solution (0.1 ml) and water (0.2 ml). The resulting mixture was stirred for 30 min and filtered. A filter cake was rinsed with THF (5 ml) and the filtrate was concentrated in vacuum to afford (2R,5S)-1-isobutyl-2-methyl-5-phenyl-piperazine (0.1 g, 430.36 umol, 75.73% yield). LCMS(ESI): [M]⁺ m/z: calcd 232.2; found 233.2; Rt=0.817 min.

### Step 9: Synthesis of tert-butyl (3-cyclopropyl-5-(2-((2S,5R)-4-isobutyl-5-methyl-2-phenylpiperazin-1-yl)-2-oxoacetamido)pyridin-2-yl)carbamate

Prepared as described in Scheme 1.1, step 1.1.10A. Yield: 0.23 g of crude.

LCMS(ESI): [M]⁺ m/z: calcd 535.2; found 536.2; Rt=1.052 min.

### Step 10: Synthesis of N-(6-amino-5-cyclopropylpyridin-3-yl)-2-((2S,5R)-4-isobutyl-5-methyl-2-phenylpiperazin-1-yl)-2-oxoacetamide

Prepared as described in Scheme 1.1, method A. Yield: 40 mg (21.39%). HPLC conditions: Column: YMC Triart C18 100*20 mm, 5 microM; 0-5 min 50-100% MeOH/water+0.1% NH₄OH 30 ml/min; (loading pump 4 ml/min MeOH).

Compound 122: ¹H NMR (600 MHz, DMSO-d₆) δ (ppm) 0.42-0.47 (m, 2H), 0.79-0.83 (m, 6H), 0.85-0.90 (m, 2H), 0.92-0.96 (m, 3H), 1.61-1.69 (m, 1H), 1.69-1.77 (m, 1H), 2.09-2.14 (m, 2H), 2.82-2.95 (m, 2H), 3.07-3.15 (m, 1H), 3.31-3.36 (m, 1H), 3.49-4.02 (m, 1H), 5.10-5.49 (m, 1H), 5.73-5.77 (m, 2H), 7.21-7.28 (m, 1H), 7.30-7.36 (m, 3H), 7.43-7.51 (m, 2H), 8.01-8.06 (m, 1H), 10.46 (br s, 1H). LCMS(ESI): [M]⁺ m/z: calcd 435.2; found 436.2; Rt=1.907 min.

### Example 27. Compound 98 N-(6-amino-5-cyclopropylpyridin-3-yl)-2-((2S,5R)-4-isobutyryl-5-methyl-2-phenylpiperazin-1-yl)-2-oxoacetamide

Prepared as described in Scheme 1.1.

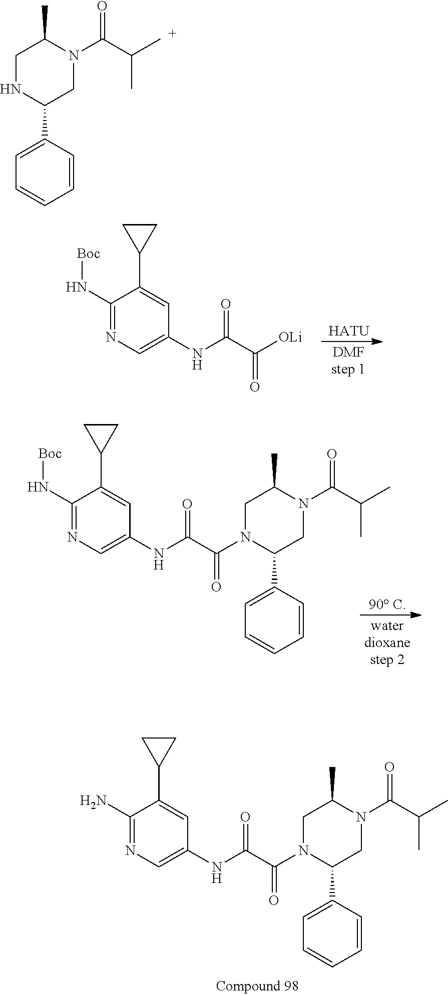

### Step 1: Synthesis of tert-butyl (3-cyclopropyl-5-(2-((2S,5R)-4-isobutyryl-5-methyl-2-phenylpiperazin-1-yl)-2-oxoacetamido)pyridin-2-yl)carbamate

Prepared as described in Scheme 1.1, step 1.1.10A. Yield: 0.33 g of crude.

LCMS(ESI): [M]⁺ m/z: calcd 549.2; found 550.2; Rt=1.353 min.

### Step 2: Synthesis of N-(6-amino-5-cyclopropylpyridin-3-yl)-2-((2S,5R)-4-isobutyryl-5-methyl-2-phenylpiperazin-1-yl)-2-oxoacetamide

Prepared as described in Boc Group Deprotection Methods, method A. Yield: 160 mg (59.28%). HPLC conditions: Column: YMC Triart C18 100*20 mm, 5 microM; 0-4 min 40-70% MeOH/water+0.1% NH₄OH 30 ml/min; (loading pump 4 ml/min MeOH).

Compound 98: ¹H NMR (600 MHz, DMSO-d₆) δ (ppm) 0.38-0.51 (m, 2H), 0.70-0.77 (m, 1H), 0.83-0.90 (m, 4H), 0.91-0.94 (m, 1H), 0.97-1.02 (m, 2H), 1.08-1.12 (m, 1H), 1.21-1.28 (m, 2H), 1.57-1.71 (m, 1H), 2.69-2.79 (m, 1H), 3.11-3.26 (m, 1H), 3.45-3.70 (m, 1H), 3.70-4.12 (m, 1H), 4.14-4.34 (m, 1H), 4.44-5.00 (m, 1H), 5.34-5.71 (m, 1H), 5.71-5.82 (m, 2H), 7.11-7.29 (m, 2H), 7.31-7.40 (m, 4H), 7.80-8.15 (m, 1H), 10.27-10.59 (m, 1H). LCMS(ESI): [M]⁺ m/z: calcd 449.2; found 450.2; Rt=2.129 min.

### Example 28. Compound 143 N-(6-amino-5-methylpyridin-3-yl)-2-((2S,5R)-5-methyl-4-(1-methylcyclopropanecarbonyl)-2-phenylpiperazin-1-yl)-2-oxoacetamide

Prepared as described in Scheme 1.1.

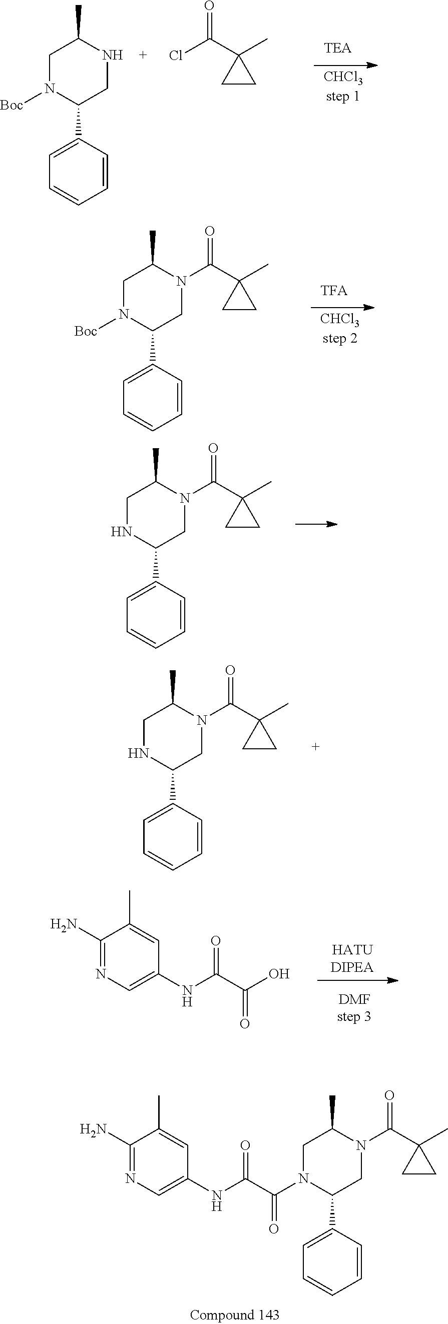

### Step 1: Synthesis of (2S,5R)-tert-butyl 5-methyl-4-(1-methylcyclopropanecarbonyl)-2-phenylpiperazine-1-carboxylate

Prepared as described in Scheme 1.1, step 1.1.8A. Yield: 0.35 g (96.37%).

LCMS(ESI): [M-Boc]⁺ m/z: calcd 258.2; found 259.2; Rt=1.391 min.

### Step 2: Synthesis of ((2R,5S)-2-methyl-5-phenylpiperazin-1-yl)(1methylcyclopropyl) methanone

Prepared as described in Scheme 1.1, step 1.1.9. Yield: 0.2 g (79.29%). LCMS(ESI): [M]⁺ m/z: calcd 258.2; found 259.2; Rt=0.692 min.

### Step 3: Synthesis of N-(6-amino-5-methylpyridin-3-yl)-2-((2S,5R)-5-methyl-4-(1-methylcyclopropanecarbonyl)-2-phenylpiperazin-1-yl)-2-oxoacetamide

Prepared as described in Scheme 1.1, step 1.1.10B. Yield: 80 mg (47.46%). HPLC conditions: Column: YMC Triart C18 100*20 mm, 5 microM; 0-5 min 20-70% MeOH/water+0.1% NH₄OH 30 ml/min; (loading pump 4 ml/min MeOH).

Compound 143: ¹H NMR (600 MHz, DMSO-d₆) δ (ppm) 0.38-0.52 (m, 2H), 0.55-0.84 (m, 2H), 1.03-1.06 (m, 3H), 1.16-1.33 (m, 3H), 1.99-2.04 (m, 3H), 2.77-3.29 (m, 2H), 3.60-4.07 (m, 1H), 4.07-4.99 (m, 2H), 5.29-5.61 (m, 1H), 5.61-5.79 (m, 2H), 7.23-7.29 (m, 2H), 7.32-7.37 (m, 3H), 7.46-7.57 (m, 1H), 7.99-8.10 (m, 1H), 10.58 (s, 1H).

LCMS(ESI): [M]⁺ m/z: calcd 435.2; found 436.2; Rt=2.403 min.

### Example 29. Compound 97 N-(6-amino-5-cyclopropylpyridin-3-yl)-2-((2S,5R)-5-methyl-2-phenyl-4-pivaloylpiperazin-1-yl)-2-oxoacetamide

Prepared as described in Scheme 1.1.

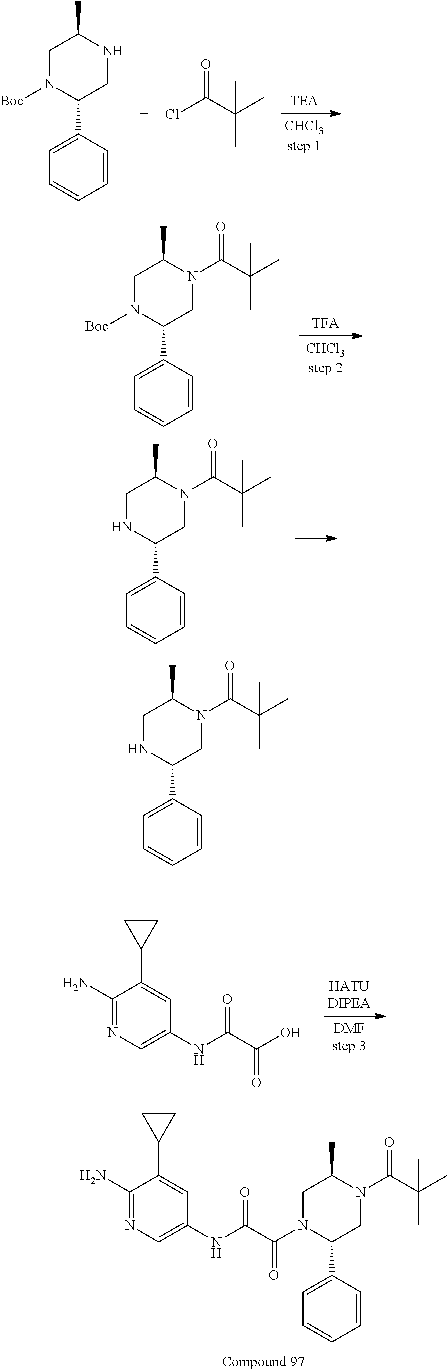

### Step 1: Synthesis of (2S,5R)-tert-butyl 5-methyl-2-phenyl-4-pivaloylpiperazine-1-carboxylate

Prepared as described in Scheme 1.1, step 1.1.8A. Yield: 0.65 g (99.67%). LCMS(ESI): [M-Boc]⁺ m/z: calcd 260.2; found 261.2; Rt=1.597 min.

### Step 2: Synthesis of 2,2-dimethyl-1-((2R,5S)-2-methyl-5-phenylpiperazin-1-yl)propan-1-one

Prepared as described in Scheme 1.1, step 1.1.9. Yield: 0.46 g (97.98%).

LCMS(ESI): [M]⁺ m/z: calcd 260.2; found 261.2; Rt=0.910 min.

### Step 3: Synthesis of tert-butyl (3-cyclopropyl-5-(2-((2S,5R)-5-methyl-2-phenyl-4-pivaloylpiperazin-1-yl)-2-oxoacetamido)pyridin-2-yl)carbamate

Prepared as described in Scheme 1.1, step 1.1.10A. Yield: 0.17 g of crude.

LCMS(ESI): [M]⁺ m/z: calcd 563.2; found 564.2; Rt=1.272 min.

### Step 4: Synthesis of N-(6-amino-5-cyclopropylpyridin-3-yl)-2-((2S,5R)-5-methyl-2-phenyl-4-pivaloylpiperazin-1-yl)-2-oxoacetamide

Prepared as described in Boc Group Deprotection Methods, method A. Yield: 58 mg (41.49%). HPLC conditions: Column: YMC Triart C18 100*20 mm, 5 microM; 0-5 min 40-90% MeOH/water+0.1% NH₄OH 30 ml/min; (loading pump 4 ml/min MeOH).

Compound 97: ¹H NMR (600 MHz, DMSO-d₆) δ (ppm) 0.43-0.48 (m, 2H), 0.85-0.90 (m, 2H), 1.03-1.10 (m, 1OH), 1.17-1.30 (m, 4H), 1.63-1.70 (m, 1H), 3.60-4.05 (m, 1H), 4.39-5.10 (m, 2H), 5.30-5.66 (m, 1H), 5.73-5.79 (m, 2H), 7.25-7.28 (m, 2H), 7.31-7.37 (m, 4H), 7.99-8.07 (m, 1H), 10.46-10.59 (m, 1H). LCMS(ESI): [M]⁺ m/z: calcd 463.2; found 464.2; Rt=2.894 min.

### Example 30. Compound 99 N-(6-amino-5-methylpyridin-3-yl)-2-((2S,5R)-5-methyl-2-phenyl-4-pivaloylpiperazin-1-yl)-2-oxoacetamide

Prepared as described in Scheme 1.1.

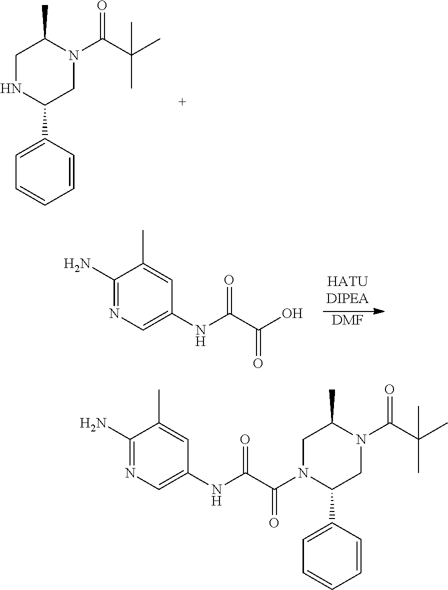

Prepared as described in 0, step 1.1.10B. Yield: 50 mg (37.19%). HPLC conditions: Column: YMC Triart C18 100*20 mm, 5 microM; 0-5 min 30-80% MeOH/water+0.1% NH₄OH 30 ml/min; (loading pump 4 ml/min MeOH).

Compound 99: ¹H NMR (600 MHz, DMSO-d₆) δ (ppm) 1.05-1.10 (m, 9H), 1.21-1.29 (m, 3H), 1.99-2.05 (m, 3H), 3.58-4.05 (m, 1H), 4.42-5.08 (m, 4H), 5.30-5.69 (m, 3H), 7.25-7.29 (m, 2H), 7.30-7.37 (m, 3H), 7.45-7.50 (m, 1H), 7.96-8.03 (m, 1H), 10.58 (s, 1H).

LCMS(ESI): [M]⁺ m/z: calcd 437.2; found 438.2; Rt=2.709 min.

### Example 31. Compound 151, Compound 95, Compound 82, Compound 92 isopropyl 4-(2-((6-amino-5-cyclopropylpyridin-3-yl)amino)-2-oxoacetyl)-5-(4-fluorophenyl)-2-methylpiperazine-1-carboxylate

Prepared as described in Scheme 1.1.

### Step 1: Synthesis of isopropyl 5-(4-fluorophenyl)-2-methyl-4-(2,2,2-trifluoroacetyl)piperazine-1-carboxylate

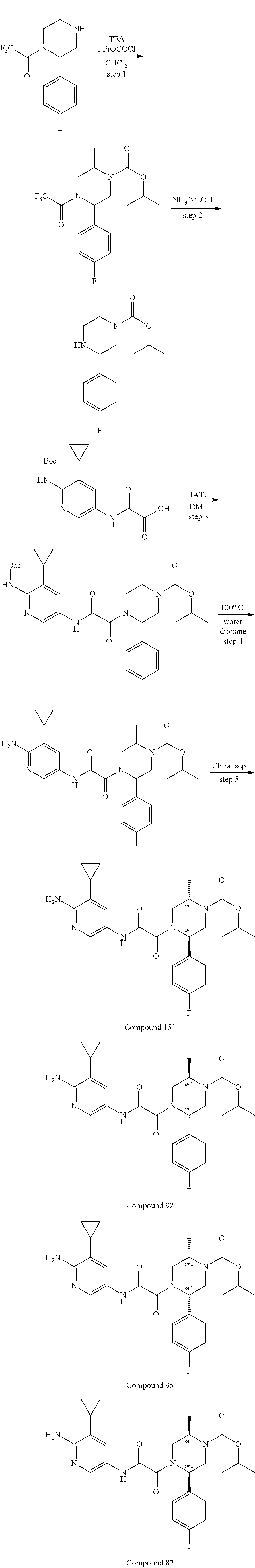

2,2,2-Trifluoro-1-[2-(4-fluorophenyl)-5-methyl-piperazin-1-yl]ethanone (0.706 g, 2.43 mmol) and TEA (984.51 mg, 9.73 mmol, 1.36 mL) were mixed together in chloroform (10 mL) and the resulting mixture was cooled to −5° C. in an ice/MeOH bath. isopropyl carbonochloridate (894.25 mg, 7.30 mmol, 828.01 uL) was added to the previous mixture and the resulting mixture was allowed to warm to rt and stirred overnight. Water (20 ml) was added to the reaction mixture and an organic layer was separated, dried over Na₂SO₄, filtered and evaporated. Conversion rate was 48% by LCMS. The residue was re-dissolved in chloroform (10 mL) and TEA (1.48 g, 14.59 mmol, 2.03 mL) was added to the resulting solution. The resulting mixture was cooled to −5° C. in an ice/MeOH bath and isopropyl carbonochloridate (1.49 g, 12.16 mmol) was added to the previous mixture and the resulting mixture was allowed to warm to rt and stirred overnight. Water (20 ml) was added to the reaction mixture and an organic layer was separated, dried over Na₂SO₄, filtered, and evaporated to obtain isopropyl 5-(4-fluorophenyl)-2-methyl-4-(2,2,2-trifluoroacetyl)piperazine-1-carboxylate (0.9 g, 2.39 mmol, 98.32% yield).

LCMS(ESI): [M]⁺ m/z: calcd 376.2; found 377.2; Rt=1.423 min.

### Step 2: Synthesis of isopropyl 5-(4-fluorophenyl)-2-methylpiperazine-1-carboxylate

iso—Propyl 5-(4-fluorophenyl)-2-methyl-4-(2,2,2-trifluoroacetyl)piperazine-1-carboxylate (945 mg, 2.51 mmol) in NH₃/MeOH (30 mL) was stirred overnight. The reaction mixture was concentrated in vacuum. Conversion rate was 58% by LCMS. The residue was dissolved in NH₃/MeOH (30 mL) and the resulting solution was stirred overnight. The reaction mixture was concentrated in vacuum. Conversion rate was 85% by LCMS. The residue was dissolved in NH₃/MeOH (30 mL) and the resulting solution was stirred overnight. The reaction mixture was concentrated in vacuum to obtain isopropyl 5-(4-fluorophenyl)-2-methyl-piperazine-1-carboxylate (756 mg, crude). LCMS(ESI): [M]⁺ m/z: calcd 280.2; found 281.2; Rt=0.805 min.

### Step 3: Synthesis of isopropyl 4-(2-((6-((tert-butoxycarbonyl)amino)-5-cyclopropylpyridin-3-yl)amino)-2-oxoacetyl)-5-(4-fluorophenyl)-2-methylpiperazine-1-carboxylate

Prepared as described in Scheme 1.1, step 1.1.10A. Yield: 1.5 g of crude.

LCMS(ESI): [M]⁺ m/z: calcd 583.2; found 584.2; Rt=1.344 min.

### Step 4: Synthesis of isopropyl 4-(2-((6-amino-5-cyclopropylpyridin-3-yl)amino)-2-oxoacetyl)-5-(4-fluorophenyl)-2-methylpiperazine-1-carboxylate

Prepared as described in Boc Group Deprotection Methods, method A. Yield: 297.5 mg (26.6%). LCMS(ESI): [M]⁺ m/z: calcd 483.2; found 484.2; Rt=1.045 min.

### Step 5: Chiral Separation

Racemic isopropyl 4-[2-[(6-amino-5-cyclopropyl-3-pyridyl)amino]-2-oxo-acetyl]-5-(4-fluorophenyl)-2-methyl-piperazine-1-carboxylate (242.1 mg, 500.69 umol) was chiral separated (Chiralcel OD-H(250*20 mm, 5 mkm), Hexane-IPA-MeOH, 90-5-5, 13 ml/min, RT(Compound 95)=56.043 min, RT(Compound 82)=79.39 min; Chiralpak AS-H(250*20, 5 mkm), Hexane-IPA-MeOH,90-5-5, 12 ml/min, RT(Compound 151)=34.779 min, RT(Compound 92)=51.507 min) to obtain isopropyl (2S,5R)-4-[2-[(6-amino-5-cyclopropyl-3-pyridyl)amino]-2-oxo-acetyl]-5-(4-fluorophenyl)-2-methyl-piperazine-1-carboxylate (48.89 mg, 101.11 umol, 20.19% yield), isopropyl (2R,5S)-4-[2-[(6-amino-5-cyclopropyl-3-pyridyl)amino]-2-oxo-acetyl]-5-(4-fluorophenyl)-2-methyl-piperazine-1-carboxylate (49.03 mg, 101.40 umol, 20.25% yield), isopropyl (2S,5S)-4-[2-[(6-amino-5-cyclopropyl-3-pyridyl)amino]-2-oxo-acetyl]-5-(4-fluorophenyl)-2-methyl-piperazine-1-carboxylate (44.97 mg, 93.00 umol, 18.57% yield), isopropyl (2R,5R)-4-[2-[(6-amino-5-cyclopropyl-3-pyridyl)amino]-2-oxo-acetyl]-5-(4-fluorophenyl)-2-methyl-piperazine-1-carboxylate (45.75 mg, 94.62 umol, 18.90% yield).

Rel Time for Compound 151 in analytical conditions (Chiralcel AS-H (250*4.6, 5 mkm), Hexane-IPA-MeOH, 90-5-5, 0.6 ml/min as mobile phase) 34.39 min and for Compound 92 43.49 min.

Rel Time for Compound 95 in analytical conditions (Chiralcel OD-H (250*4.6, 5 mkm), Hexane-IPA-MeOH, 70-15-15, 0.6 ml/min as mobile phase) 12.75 min and for Compound 82 16.72 min.

Compound 151: Retention time: 34.39 min

1H NMR (DMSO-d₆, 600 MHz): δ (ppm) 0.45 (m, 2H), 0.87 (m, 2H), 1.14 (m, 9H), 1.66 (m, 1H), 2.93 (m, 1H), 3.43 (m, 1H), 3.79 (dd, 1H), 4.15 (m, 1H), 4.41 (m, 1H), 4.74 (m, 1H), 5.66 (m, 3H), 7.20 (m, 2H), 7.34 (m, 3H), 8.01 (m, 1H), 10.50 (m, 1H) LCMS(ESI): [M]⁺ m/z: calcd 483.2; found 484.2; Rt=2.778 min.

Compound 92: Retention time: 43.49 min

1H NMR (DMSO-d₆, 600 MHz): δ (ppm) 0.45 (m, 2H), 0.88 (m, 2H), 1.14 (m, 9H), 1.66 (m, 1H), 2.98 (m, 1H), 3.45 (m, 1H), 3.79 (dd, 1H), 4.15 (m, 1H), 4.41 (m, 1H), 4.74 (m, 1H), 5.66 (m, 3H), 7.21 (m, 2H), 7.34 (m, 3H), 8.01 (m, 1H), 10.50 (m, 1H) LCMS(ESI): [M]⁺ m/z: calcd 483.2; found 484.2; Rt=2.784 min.

Compound 95: Retention time: 12.75 min

1H NMR (DMSO-d₆, 600 MHz): δ (ppm) 0.43 (m, 2H), 0.87 (m, 2H), 1.17 (m, 1OH), 1.64 (m, 1H), 3.46 (m, 1H), 4.17 (m, 3H), 4.89 (m, 2H), 5.76 (m, 2H), 7.19 (m, 2H), 7.40 (m, 3H), 8.07 (m, 1H), 10.22 (m, 1H)

LCMS(ESI): [M]⁺ m/z: calcd 483.2; found 484.2; Rt=2.804 min.

Compound 82: Retention time: 16.72 min

1H NMR (DMSO-d₆, 600 MHz): δ (ppm) 0.45 (m, 2H), 0.87 (m, 2H), 1.17 (m, 9H), 1.65 (m, 1H), 3.18 (m, 1H), 3.46 (m, 1H), 4.15 (m, 3H), 4.86 (m, 2H), 5.74 (m, 2H), 7.16 (m, 2H), 7.39 (m, 3H), 8.06 (m, 1H), 10.21 (m, 1H) LCMS(ESI): [M]⁺ m/z: calcd 483.2; found 484.2; Rt=2.821 min.

### Example 32. Compound 4 rac-N-(6-amino-5-cyclopropylpyridin-3-yl)-2-((2R,53)-4-isobutyryl-5-methyl-2-(4-(4-methylpiperazin-1-yl)phenyl)piperazin-1-yl)-2-oxoacetamide

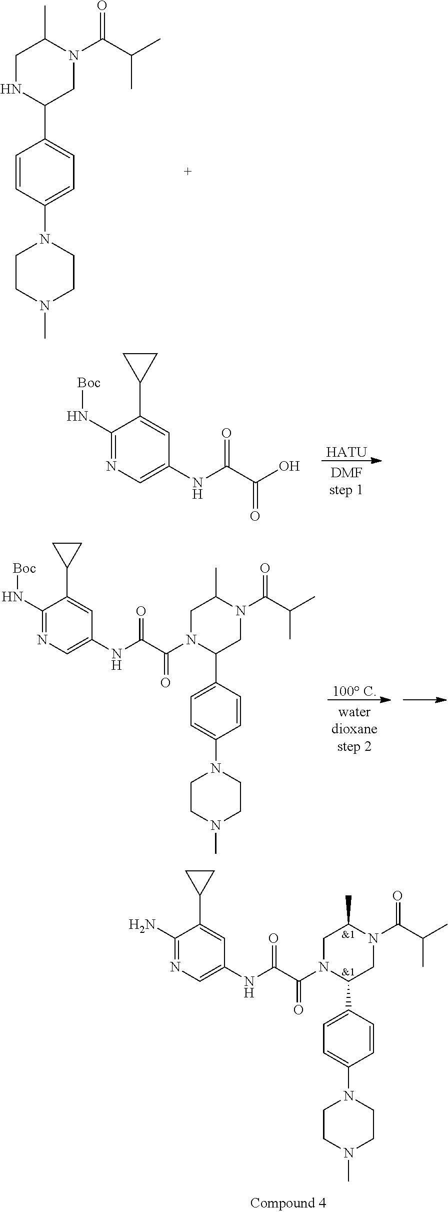

### Step 1: Synthesis of tert-butyl (3-cyclopropyl-5-(2-(4-isobutyryl-5-methyl-2-(4-(4-methylpiperazin-1-yl)phenyl)piperazin-1-yl)-2-oxoacetamido)pyridin-2-yl)carbamate

2-Methyl-1-[2-methyl-5-[4-(4-methylpiperazin-1-yl)phenyl]piperazin-1-yl]propan-1-one (303.2 mg, 880.13 umol) and HATU (401.58 mg, 1.06 mmol) were mixed together in DMF (12 mL) and the resulting mixture was stirred for 15 min. 2-[[6-(tert-Butoxycarbonylamino)-5-cyclopropyl-3-pyridyl]amino]-2-oxo-acetic acid (288.92 mg, 880.13 umol, Li+) was added to the previous mixture and the resulting mixture was stirred overnight. The reaction mixture was concentrated in vacuum and the residue was purified by HPLC (2-10 min 50-80% MeCN 30 ml/min (loading pump 4 ml MeCN column: SunFire 100*19 mm, 5 microM) to obtain tert-butyl N-[3-cyclopropyl-5-[[2-[5-methyl-2-[4-(4-methylpiperazin-1-yl)phenyl]-4-(2-methylpropanoyl)piperazin-1-yl]-2-oxo-acetyl]amino]-2-pyridyl]carbamate (147.4 mg, 227.54 umol, 25.85% yield).

LCMS(ESI): [M]⁺ m/z: calcd 647.2; found 648.2; Rt=0.976 min.

### Step 2: Synthesis of rac-N-(6-amino-5-cyclopropylpyridin-3-yl)-2-((2R,5S)-4-isobutyryl-5-methyl-2-(4-(4-methylpiperazin-1-yl)phenyl)piperazin-1-yl)-2-oxoacetamide

tert-Butyl N-[3-cyclopropyl-5-[[2-[5-methyl-2-[4-(4-methylpiperazin-1-yl)phenyl]-4-(2-methyl propanoyl)piperazin-1-yl]-2-oxo-acetyl]amino]-2-pyridyl]carbamate (147.4 mg, 227.54 umol) was dissolved in dioxane (1 mL) and water (1 mL) was added thereto. The resulting mixture was heated at 100° C. overnight. The reaction mixture was concentrated in vacuum and purified by HPLC (2-10 min 30-80% MeCN 30 ml/min; loading pump 4 ml/min MeOH, column SunFire 19*100 mm) to obtain N-(6-amino-5-cyclopropyl-3-pyridyl)-2-[(2S,5R)-5-methyl-2-[4-(4-methylpiperazin-1-yl)phenyl]-4-(2-methylpropanoyl) piperazin-1-yl]-2-oxo-acetamide (15.7 mg, 28.67 umol, 12.60% yield).

¹H NMR (600 MHz, DMSO-d₆) δ (ppm) 0.43 (m, 2H), 0.88 (m, 8H), 0.99 (t, 3H), 1.12 (m, 3H), 1.66 (m, 1H), 2.21 (m, 4H), 2.43 (m, 2H), 2.77 (m, 1H), 3.10 (m, 4H), 3.90 (m, 2H), 4.88 (m, 1H), 5.72 (m, 3H), 6.86 (m, 2H), 7.12 (m, 2H), 7.36 (m, 1H), 8.06 (s, 1H), 10.54 (m, 1H).

LCMS(ESI): [M]⁺ m/z: calcd 547.2; found 548.2; Rt=1.834 min.

### Example 33. Compound 7 N-(6-amino-5-methylpyridin-3-yl)-2-((2S,5R)-5-methyl-2-(3-(4-methylpiperazin-1-yl)phenyl)-4-pivaloylpiperazin-1-yl)-2-oxoacetamide

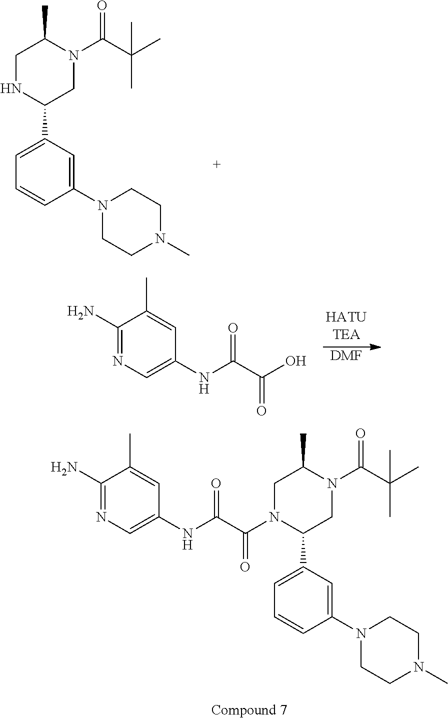

The Synthesis of 2,2-dimethyl-1-((2R,5S)-2-methyl-5-(3-(4-methylpiperazin-1-yl)phenyl)piperazin-1-yl)propan-1-one is given in by the following.

tert-Butyl (2S,5R)-4-(2,2-dimethylpropanoyl)-5-methyl-2-[3-(4-methylpiperazin-1-yl)phenyl]piperazine-1-carboxylate (1.15 g, 2.51 mmol) was dissolved in TFA (10 mL) and DCM (10 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated. The obtained product was used in the next step without further purification. 2,2-Dimethyl-1-[(2R,5S)-2-methyl-5-[3-(4-methylpiperazin-1-yl)phenyl]piperazin-1-yl]propan-1-one (0.9 g, crude) was obtained as a brown gum. LCMS(ESI): [M]⁺ m/z: calcd 358.2; found 359.2; Rt=0.699 min.

Prepared by Scheme 1.1, step 10A. Yield: 19 mg (6.36%).

HPLC conditions: Column: SunFire C18 100*19 mm, 5 microM; 2-10 min 0-25% water-MeOH+NH₃ 30 ml/min; (loading pump 4 ml/min MeOH+NH₃).

Compound 7: ¹H NMR (500 MHz, DMSO-d₆) δ (ppm) 1.09 (m, 11H), 1.24 (m, 3H), 2.02 (m, 3H), 2.19 (m, 3H), 2.41 (m, 4H), 3.06 (m, 5H), 3.98 (m, 1H), 4.45 (m, 1H), 5.63 (m, 3H), 6.74 (m, 3H), 7.16 (m, 1H), 7.48 (s, 1H), 8.02 (s, 1H), 10.58 (s, 1H).

LCMS(ESI): [M]⁺ m/z: calcd 535.2; found 536.2; Rt=1.902 min.

### Example 34. Compound 124 N-(4-amino-1H-pyrazolo[4,3-c]pyridin-7-yl)-2-((2S,5R)-5-methyl-2-phenyl-4-pivaloylpiperazin-1-yl)-2-oxoacetamide

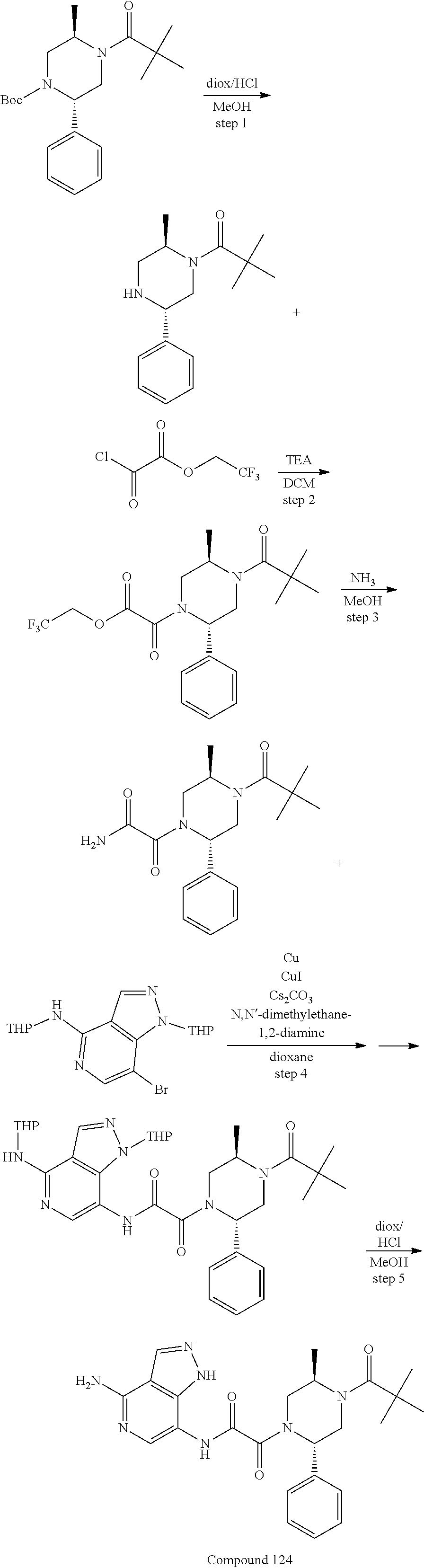

The synthesis of (2S,5R)-tert-butyl 5-methyl-2-phenyl-4-pivaloylpiperazine-1-carboxylate is given by Scheme 1.1, step 7.

### Step 1: Synthesis of 2,2-dimethyl-1-((2R,5S)-2-methyl-5-phenylpiperazin-1-yl)propan-1-one

The stirred solution of tert-butyl (2S,5R)-4-(2,2-dimethylpropanoyl)-5-methyl-2-phenyl-piperazine-1-carboxylate (480.00 mg, 1.33 mmol) in MeOH (10 mL) and diox/HCl (5 mL) was allowed to stir at 25° C. for 4 hr. Upon completion, the reaction mixture was evaporated, the crude product was quenched with water (20 mL) and neutralized by NaHCO₃ to pH=8. The aqueous phase was extracted with CHCl₃ (2*20 mL). The combined organic phase was dried over Na₂SO₄ and concentrated under reduced pressure. The desired product 2,2-dimethyl-1-[(2R,5S)-2-methyl-5-phenyl-piperazin-1-yl]propan-1-one (0.34 g, 1.31 mmol, 98.07% yield) was isolated.

LCMS(ESI): [M]⁺ m/z: calcd 260.2; found 261.2; Rt=0.783 min

### Step 2: Synthesis of 2,2,2-trifluoroethyl 2-((2S,5R)-5-methyl-2-phenyl-4-pivaloylpiperazin-1-yl)-2-oxoacetate

2,2-Dimethyl-1-[(2R,5S)-2-methyl-5-phenyl-piperazin-1-yl]propan-1-one (0.34 g, 1.31 mmol) and TEA (246.84 mg, 2.44 mmol, 340.00 L) were mixed together in DCM (5 mL) and the resulting solution was cooled to 5° C. in an ice bath. 2,2,2-Trifluoroethyl 2-chloro-2-oxo-acetate (340.00 mg, 1.78 mmol) was added dropwise to the previous solution and the resulting mixture was allowed to warm to rt and stirred overnight. After completion the reaction mixture was diluted with CHCl₃ (5 ml) and the resulting solution was washed with water (2*10 ml), dried over Na₂SO₄, filtered and evaporated. The desired product 2,2,2-trifluoroethyl 2-[(2S,5R)-4-(2,2-dimethylpropanoyl)-5-methyl-2-phenyl-piperazin-1-yl]-2-oxo-acetate (0.54 g, 1.30 mmol, 99.79% yield) was isolated.

LCMS(ESI): [M]⁺ m/z: calcd 414.2; found 415.2; Rt=1.330 min.

### Step 3: Synthesis of 2,2,2-trifluoroethyl 2-((2S,5R)-5-methyl-2-phenyl-4-pivaloylpiperazin-1-yl)-2-oxoacetate

2,2-Dimethyl-1-[(2R,5S)-2-methyl-5-phenyl-piperazin-1-yl]propan-1-one (0.34 g, 1.31 mmol) and TEA (246.84 mg, 2.44 mmol, 340.00 L) were mixed together in DCM (5 mL) and the resulting solution was cooled to 5° C. in an ice bath. 2,2,2-Trifluoroethyl 2-chloro-2-oxo-acetate (340.00 mg, 1.78 mmol) was added dropwise to the previous solution and the resulting mixture was allowed to warm to rt and stirred overnight. After completion the reaction mixture was diluted with CHCl₃ (5 ml) and the resulting solution was washed with water (2*10 ml), dried over Na₂SO₄, filtered and evaporated. The desired product 2,2,2-trifluoroethyl 2-[(2S,5R)-4-(2,2-dimethylpropanoyl)-5-methyl-2-phenyl-piperazin-1-yl]-2-oxo-acetate (0.54 g, 1.30 mmol, 99.79% yield) was isolated.

LCMS(ESI): [M]⁺ m/z: calcd 331.2; found 332.2; Rt=1.121 min.

### Step 4: Synthesis of 2-((2S,5R)-5-methyl-2-phenyl-4-pivaloylpiperazin-1-yl)-2-oxo-N-(1-(tetrahydro-2H-pyran-2-yl)-4-((tetrahydro-2H-pyran-2-yl)amino)-1H-pyrazolo[4,3-c]pyridin-7-yl)acetamide)

7-Bromo-N,1-di(tetrahydropyran-2-yl)pyrazolo[4,3-c]pyridin-4-amine (0.2 g, 524.57 mol), 2-oxo-2-[(2S,5R)-4-(2,2-dimethylpropanoyl)-5-methyl-2-phenyl-piperazin-1-yl]acetamide (0.05 g, 150.87 mol), copper (0.01 g, 157.37 mol), copper (I) iodide (0.03 g, 157.52 mol, 5.34 L) and cesium carbonate (0.1 g, 306.92 mol) were mixed together in dioxane (5 mL). The resulting suspension was degassed with argon at 50° C. for 0.1 hr. (¹R,²R)—N¹,N²-Dimethylcyclohexane-1,2-diamine (45.00 mg, 316.36 μmol, 0.05 mL) was added thereto and the resulting mixture was stirred for 16 h at 100° C. After completion the reaction mixture was filtered and the filtrate was concentrated in vacuum. The desired product 2-oxo-2-[(2S,5R)-4-(2,2-dimethylpropanoyl)-5-methyl-2-phenyl-piperazin-1-yl]-N-[1-tetrahydropyran-2-yl-4-(tetrahydropyran-2-ylamino)pyrazolo[4,3-c]pyridin-7-yl]acetamide (95 mg, 150.37 mol, 99.67% yield) was isolated.

LCMS(ESI): [M-THP]⁺ m/z: calcd 547.2; found 548.2; Rt=1.049 min.

### Step 5: Synthesis of N-(4-amino-1H-pyrazolo[4,3-c]pyridin-7-yl)-2-((2S,5R)-5-methyl-2-phenyl-4-pivaloylpiperazin-1-yl)-2-oxoacetamide (Compound 124)

The solution of 2-oxo-2-[(2S,5R)-4-(2,2-dimethylpropanoyl)-5-methyl-2-phenyl-piperazin-1-yl]-N-[1-tetrahydropyran-2-yl-4-(tetrahydropyran-2-ylamino)pyrazolo[4,3-c]pyridin-7-yl]acetamide (95.00 mg, 150.37 μmol) in diox/HCl (5 mL) and MeOH (5 mL) was stirred at 25° C. for 16 hr. Upon completion, the reaction mixture was concentrated under reduced pressure to obtain crude product. The obtained crude product was purified by reverse phase HPLC chromatography(Device (Mobile Phase, Column): SYSTEM 10-10-30% 0-1-6 min H₂O/MeCN/0.1% FA, flow: 30 ml/min (loading pump 4 ml/min MeCN) target mass 464 column: Chromatorex 18 SMB100-5T 100×19 mm 5 um) to afford product N-(4-amino-1H-pyrazolo[4,3-c]pyridin-7-yl)-2-oxo-2-[(2S,5R)-4-(2,2-dimethylpropanoyl)-5-methyl-2-phenyl-piperazin-1-yl]acetamide (2 mg, 4.31 μmol, 2.87% yield).

Compound 124: LCMS(ESI): [M]⁺ m/z: calcd 463.2; found 464.2; Rt=2.549 min.

### Example 35. Compound 110 N-(6-amino-5-ethylpyridin-3-yl)-2-((2S,5R)-5-methyl-2-(3-(4-methylpiperazin-1-yl)phenyl)-4-pivaloylpiperazin-1-yl)-2-oxoacetamide

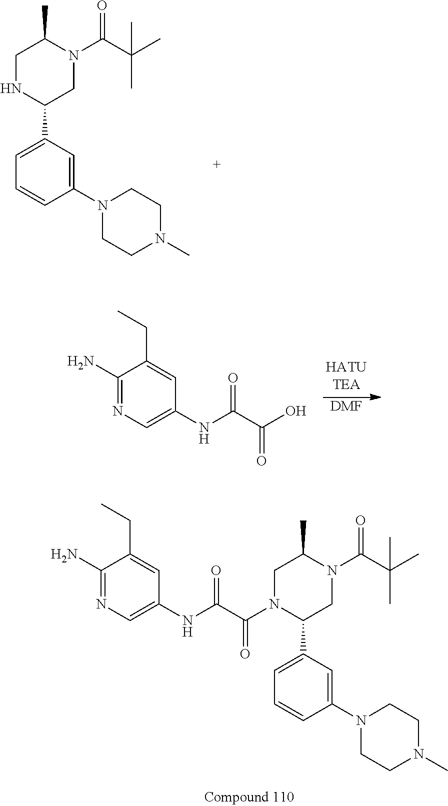

The synthesis of 2,2-dimethyl-1-((2R,5S)-2-methyl-5-(3-(4-methylpiperazin-1-yl)phenyl)piperazin-1-yl)propan-1-one is given by tert-Butyl (2S,5R)-4-(2,2-dimethylpropanoyl)-5-methyl-2-[3-(4-methylpiperazin-1-yl)phenyl]piperazine-1-carboxylate (1.15 g, 2.51 mmol) was dissolved in TFA (10 mL) and DCM (10 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated. The obtained product was used in the next step without further purification. 2,2-Dimethyl-1-[(2R,5S)-2-methyl-5-[3-(4-methylpiperazin-1-yl)phenyl]piperazin-1-yl]propan-1-one (0.9 g, crude) was obtained as a brown gum.

LCMS(ESI): [M]⁺ m/z: calcd 358.2; found 359.2; Rt=0.699 min.

Prepared by Scheme 1.1, step 10A. Yield: 14 mg (4.57%).

HPLC conditions: Column: SunFire C18 100*19 mm, 5 microM; 2-10 min 0-25% water-MeOH+NH₃ 30 ml/min; (loading pump 4 ml/min MeOH+NH₃).

Compound 110: ¹H NMR (500 MHz, DMSO-d₆) δ (ppm) 1.04 (t, 3H), 1.09 (m, 1OH), 1.24 (m, 4H), 1.62 (m, 1H), 2.19 (m, 3H), 2.38 (m, 4H), 3.07 (m, 4H), 3.98 (m, 2H), 4.35 (m, 2H), 5.65 (m, 3H), 6.65 (d, 1H), 6.82 (m, 2H), 7.16 (m, 1H), 7.49 (s, 1H), 8.05 (s, 1H), 10.59 (s, 1H).

LCMS(ESI): [M]⁺ m/z: calcd 549.2; found 550.2; Rt=2.010 min.

### Example 36. Compound 77 N-(6-amino-5-methylpyridin-3-yl)-2-((2S,5R)-5-methyl-2-(4-(4-methylpiperazin-1-yl)phenyl)-4-(1-(trifluoromethyl)cyclopropanecarbonyl)piperazin-1-yl)-2-oxoacetamide

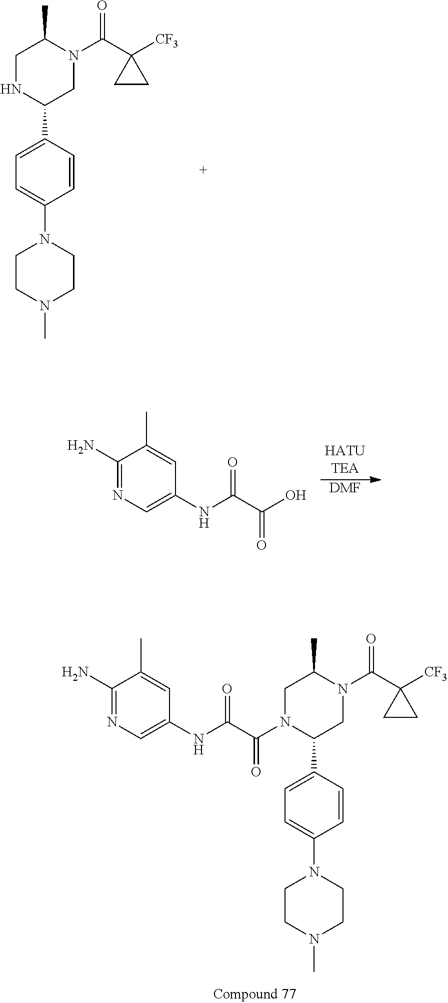

The synthesis of ((2R,5S)-2-methyl-5-(4-(4-methylpiperazin-1-yl)phenyl)piperazin-1-yl)(1-(trifluoromethyl)cyclopropyl)methanone is give by tert-Butyl (2S,5R)-5-methyl-2-[4-(4-methylpiperazin-1-yl)phenyl]-4-[1-(trifluoromethyl) cyclopropanecarbonyl]piperazine-1-carboxylate (0.8 g, 1.57 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain [(2R,5S)-2-methyl-5-[4-(4-methylpiperazin-1-yl)phenyl]piperazin-1-yl]-[1-(trifluoromethyl)cyclopropyl]methanone (0.36 g, 877.03 mol, 55.98% yield) which was used in the next step without further purification.

LCMS(ESI): [M]⁺ m/z: calcd 410.2; found 411.2; Rt=0.308 min.

Prepared by Scheme 1.1 step 10A. Yield: 55 mg (38.42%).

HPLC conditions: Column: SunFire C18 100*19 mm, 5 microM; 2-10 min 20-55% water-MeCN 30 ml/min; (loading pump 4 ml/min MeCN).

Compound 77: ¹H NMR (600 MHz, DMSO-d₆) δ (ppm) 0.93-1.01 (m, 1H), 1.09-1.35 (m, 6H), 1.92-2.04 (m, 3H), 2.16-2.21 (m, 3H), 2.38-2.43 (m, 4H), 2.64-3.23 (m, 6H), 3.63-4.85 (m, 3H), 5.13-5.72 (m, 3H), 6.80-6.93 (m, 2H), 7.02-7.21 (m, 2H), 7.45-7.57 (m, 1H), 7.97-8.07 (m, 1H), 9.85-10.70 (m, 1H).

LCMS(ESI): [M]⁺ m/z: calcd 587.2; found 588.2; Rt=1.888 min.

### Example 37. Compound 130 N-(6-amino-5-ethylpyridin-3-yl)-2-((2S,5R)-5-methyl-2-(4-(4-methylpiperazin-1-yl)phenyl)-4-pivaloylpiperazin-1-yl)-2-oxoacetamide

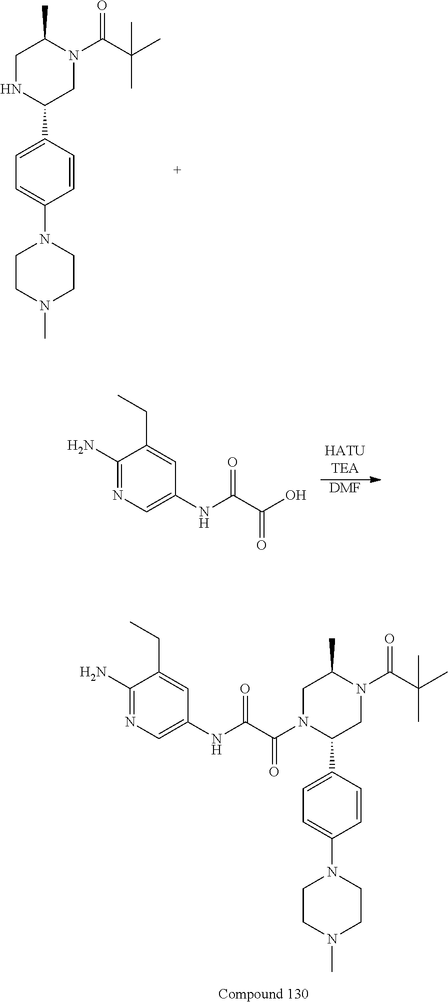

The 2,2-dimethyl-1-((2R,5S)-2-methyl-5-(4-(4-methylpiperazin-1-yl)phenyl)piperazin-1-yl)propan-1-one is given by tert-Butyl (2S,5R)-4-(2,2-dimethylpropanoyl)-5-methyl-2-[4-(4-methylpiperazin-1-yl)phenyl]piperazine-1-carboxylate (1.1 g, 2.40 mmol) was dissolved in DCM (20 mL), then TFA (7.02 g, 61.54 mmol, 4.74 mL) was added and it was stirred 16 hr at rt. The reaction mixture was poured into aq·K₂CO₃ solution and the residue was extracted with DCM (2*25 ml). Combined organic layers were fried over Na₂SO₄, filtered and concentrated in vacuum to obtain 2,2-dimethyl-1-[(2R,5S)-2-methyl-5-[4-(4-methylpiperazin-1-yl)phenyl]piperazin-1-yl]propan-1-one (230 mg, 641.53 μmol, 26.75% yield).

LCMS(ESI): [M]⁺ m/z: calcd 358.2; found 359.2; Rt=0.631 min Prepared by Scheme 1.1 step 10A. Yield: 13.1 mg (15.77%).

### Hplc Conditions:

1-st Run: Column: SunFire C18 100*19 mm, 5 microM; 2-10 min 50-100% MeOH 30 ml/min; (loading pump 4 ml/min MeOH).

2-nd Run: Column: SunFire C18 100*19 mm, 5 microM; 2-10 min 0-100% MeCN+FA 30 ml/min; (loading pump 4 ml/min MeCN).

Compound 130: ¹H NMR (600 MHz, DMSO-d₆) δ (ppm) 1.10 (m, 12H), 1.22 (m, 3H), 2.20 (m, 3H), 2.39 (m, 3H), 2.43 (m, 4H), 2.60 (m, 1H), 3.10 (m, 4H), 3.72 (m, 1H), 4.46 (m, 1H), 4.80 (m, 1H), 5.19 (m, 1H), 5.64 (m, 2H), 6.86 (m, 2H), 7.12 (m, 2H), 7.48 (s, 1H), 8.05 (s, 1H), 10.56 (s, 1H).

LCMS(ESI): [M]⁺ m/z: calcd 549.2; found 550.2; Rt=1.990 min.

### Example 38. Compound 41 N-(6-amino-5-ethylpyridin-3-yl)-2-((2S,5R)-4-isobutyryl-5-methyl-2-(3-(4-methylpiperazin-1-yl)phenyl)piperazin-1-yl)-2-oxoacetamide

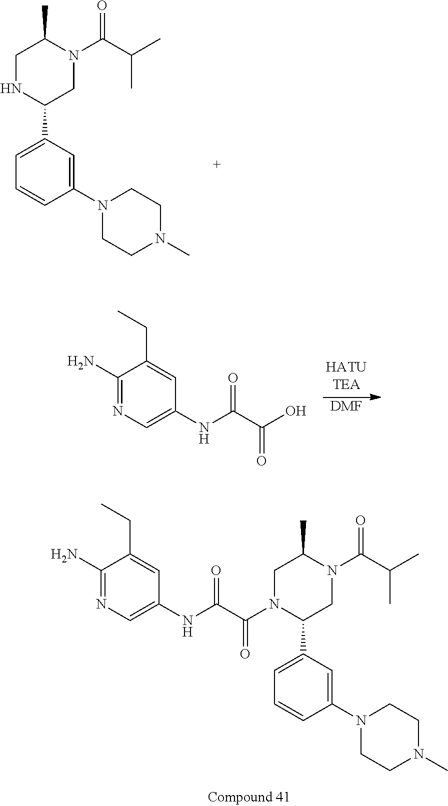

2-methyl-1-((2R,5S)-2-methyl-5-(3-(4-methylpiperazin-1-yl)phenyl)piperazin-1-yl)propan-1-one was synthesized by tert-Butyl (2S,5R)-5-methyl-2-[3-(4-methylpiperazin-1-yl)phenyl]-4-(2-methylpropanoyl)piperazine-1-carboxylate (315.00 mg, 708.49 mol) was dissolved in DCM (2 mL),TFA (2 mL) was added in one portion. The resulting mixture was allowed to stir at 20° C. for 1.5 hr. Upon completion, DCM was evaporated, 20 ml of water was added to the residue and mixture was basified with K₂CO₃ to alkaline pH. Aqueous phase was extracted with DCM (3*15 ml), combined organic layers were dried over Na₂SO₄, filtered and concentrated under reduced pressure to afford 2-methyl-1-[(2R,5S)-2-methyl-5-[3-(4-methylpiperazin-1-yl)phenyl]piperazin-1-yl]propan-1-one (180 mg, crude).

LCMS(ESI): [M]⁺ m/z: calcd 344.2; found 345.2; Rt=0.609 min.

Prepared by Scheme 1.1 step 10A. Yield: 14 mg (3%).

HPLC conditions: Column: SunFire C18 100*19 mm, 5 microM; 2-10 min 0-25% water-MeOH+NH₃ 30 ml/min; (loading pump 4 ml/min MeOH+NH₃).

Compound 41:¹H NMR (600 MHz, DMSO-d₆) δ (ppm) 0.85 (m, 5H), 1.06 (m, 9H), 1.24 (m, 2H), 2.20 (m, 3H), 2.39 (m, 2H), 2.77 (dd, 1H), 3.08 (m, 4H), 3.99 (m, 2H), 4.35 (m, 2H), 5.03 (m, 1H), 5.66 (m, 3H), 6.76 (m, 2H), 6.87 (m, 1H), 7.16 (m, 1H), 7.51 (m, 1H), 8.07 (m, 1H), 10.61 (m, 1H) LCMS(ESI): [M]⁺ m/z: calcd 535.2; found 536.2; Rt=1.848 min.

### Example 39. Compound 140 N-(6-amino-5-ethylpyridin-3-yl)-2-((2S,5R)-5-methyl-2-phenyl-4-pivaloylpiperazin-1-yl)-2-oxoacetamide

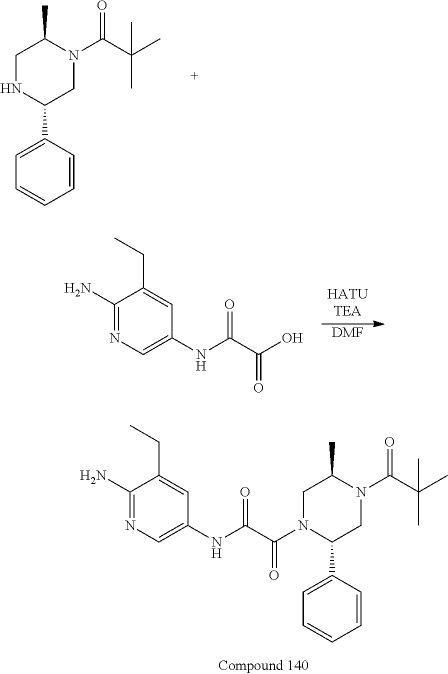

2,2-dimethyl-1-((2R,5S)-2-methyl-5-phenylpiperazin-1-yl)propan-1-one was prepared by Acheme 1.1 step 9. Yield: 0.46 g (97.98%).

LCMS(ESI): [M]⁺ m/z: calcd 260.2; found 261.2; Rt=0.910 min.

Prepared by Scheme 1.4 step 9A. Yield: 21.2 mg (12.98%).

HPLC conditions: Column: XBridge C18 100*19 mm, 5 microM; 0-6 min 30-75% MeOH-water+0.1% NH₄OH; (loading pump 4 ml/min MeOH).

Compound 140: ¹H NMR (600 MHz, DMSO-d₆) δ (ppm) 1.07 (s, 9H), 1.09-1.13 (m, 3H), 1.24 (d, 3H), 2.38-2.42 (m, 2H), 3.01-3.26 (m, 2H), 3.59-4.06 (m, 1H), 4.40-4.52 (m, 1H), 4.71-5.14 (m, 1H), 5.32-5.63 (m, 1H), 5.64-5.69 (m, 2H), 7.25-7.29 (m, 2H), 7.31-7.37 (m, 3H), 7.43-7.52 (m, 1H), 7.96-8.10 (m, 1H), 10.60 (s, 1H).

LCMS(ESI): [M]⁺ m/z: calcd 451.2; found 452.2; Rt=2.358 min.

### Example 40. Compound 44 2-oxo-N-(1H-pyrazolo[4,3-c]pyridin-7-yl)-2-[(2S,5R)-4-(2,2-dimethylpropyl)-5-methyl-2-phenyl-piperazin-1-yl]acetamide

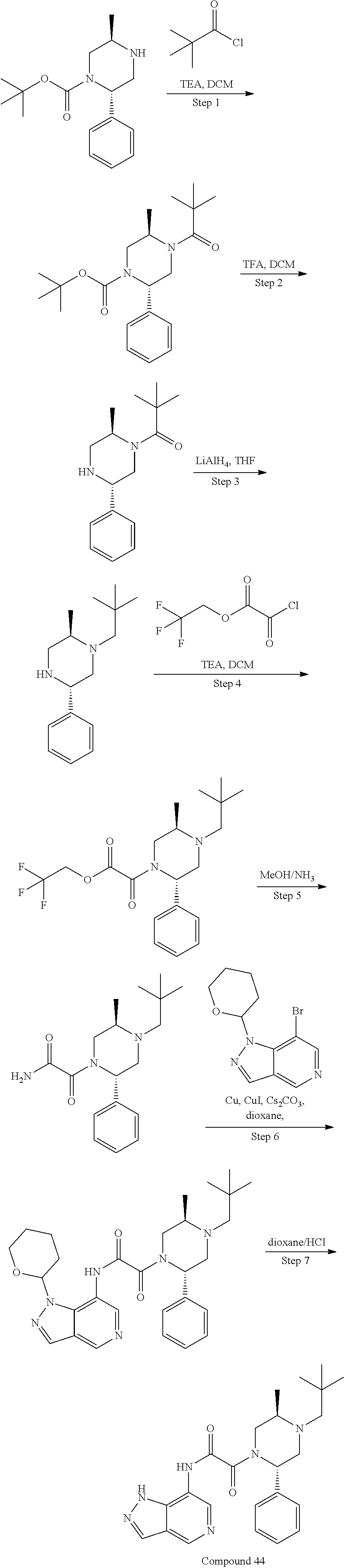

### Step 1: tert-butyl (2S,5R)-4-(2,2-dimethylpropanoyl)-5-methyl-2-phenyl-piperazine-1-carboxylate

2,2-Dimethylpropanoyl chloride (130.89 mg, 1.09 mmol, 132.88 L) was added dropwise to the solution of tert-butyl (2S,5R)-5-methyl-2-phenyl-piperazine-1-carboxylate (0.3 g, 1.09 mmol) and triethylamine (329.52 mg, 3.26 mmol, 453.89 L) in DCM (6 mL) at 25° C., and the reaction mixture was stirred at 25° C. for 24 hr. The reaction mixture was diluted by DCM (10 mL) and washed by water (3×30 mL), dried over Na₂SO₄, and evaporated under reduced pressure to afford the tert-butyl (2S,5R)-4-(2,2-dimethylpropanoyl)-5-methyl-2-phenyl-piperazine-1-carboxylate (0.5 g, 1.39 mmol, 127.78% yield) that was directly used in the next step.

LCMS(ESI): [M+1]⁺ m/z: calcd 360.2; found 361.2; Rt=1.706 min.

### Step 2: 2,2-dimethyl-1-[(2R,5S)-2-methyl-5-phenyl-piperazin-1-yl]propan-1-one

Trifluoroacetic acid (790.75 mg, 6.94 mmol, 534.29 L) was added dropwise to the solution of tert-butyl (2S,5R)-4-(2,2-dimethylpropanoyl)-5-methyl-2-phenyl-piperazine-1-carboxylate (0.5 g, 1.39 mmol) in DCM (10 mL), and the reaction mixture was stirred at 25° C. for 12 hr. Then the reaction mixture was diluted by DCM (10 mL) and washed by saturated aqueous NaHCO₃ (3*30 mL), dried over Na₂SO₄ and evaporated under reduced pressure to get 2,2-dimethyl-1-[(2R,5S)-2-methyl-5-phenyl-piperazin-1-yl]propan-1-one (0.3 g, 1.15 mmol, 83.07% yield), that was used in the next step without purification.

LCMS(ESI): [M+1]⁺ m/z: calcd 260.2; found 261.2; Rt=0.669 min.

### Step 3: (2R,5S)-1-(2,2-dimethylpropyl)-2-methyl-5-phenyl-piperazine

Lithium; alumanuide (131.19 mg, 3.46 mmol) was suspended in THF (10 mL), and the resulting suspension was heated to reflux. A solution of 2,2-dimethyl-1-[(2R,5S)-2-methyl-5-phenyl-piperazin-1-yl]propan-1-one (0.3 g, 1.15 mmol) in THF (10 mL) was added dropwise to the previous suspension, maintaining a reflux. After addition completed, the reaction mixture was refluxed for 12 hr. Then the reaction mixture was allowed to cool to r.t. Water (1 ml) was carefully added dropwise to the precooled reaction mixture. The resulting mixture was stirred for 30 min and filtered. A filter cake was rinsed with THF (3*10 ml) and the filtrate was concentrated in vacuum to obtain crude (2R,5S)-1-(2,2-dimethylpropyl)-2-methyl-5-phenyl-piperazine (0.23 g, 933.48 μmol, 81% yield) that was used in the next step without purification.

LCMS(ESI): [M+1]⁺ m/z: calcd 246.3; found 247.2; Rt=0.870 min.

### Step 4: 2,2,2-trifluoroethyl 2-oxo-2-[(2S,5R)-4-(2,2-dimethylpropyl)-5-methyl-2-phenyl-piperazin-1-yl]acetate

2,2,2-Trifluoroethyl 2-chloro-2-oxo-acetate (195 mg, 1.03 mmol) was added to the solution of (2R,5S)-1-(2,2-dimethylpropyl)-2-methyl-5-phenyl-piperazine (0.23 g, 933.48 μmol) and eriethylamine (188.92 mg, 1.87 mmol, 260.22 L) in the DCM (10 mL) at 25° C. Reaction was stirred at 25° C. for 12 hr. Then the reaction mixture was diluted by DCM (10 mL.) and washed with water (3*30 mL). The organic phase was dried over Na₂SO₄ and evaporated under reduced pressure to afford crude 2,2,2-trifluoroethyl 2-oxo-2-[(2S,5R)-4-(2,2-dimethylpropyl)-5-methyl-2-phenyl-piperazin-1-yl]acetate (0.33 g, 824.11 μmol, 88.2% yield), that was directly used in the next step.

LCMS(ESI): [M+1]⁺ m/z: calcd 400.2; found 401.2; Rt=1.589 min.

### Step 5: 2-oxo-2-[(2S,5R)-4-(2,2-dimethylpropyl)-5-methyl-2-phenyl-piperazin-1-yl]acetamide

2,2,2-Trifluoroethyl 2-oxo-2-[(2S,5R)-4-(2,2-dimethylpropyl)-5-methyl-2-phenyl-piperazin-1-yl]acetate (0.33 g, 824.11 mol) was dissolved in the MeOH/NH₃ (10 mL), and the reaction mixture was stirred at 25° C. for 12 hr. After completion the solvent was evaporated under reduced pressure to afford crude 2-oxo-2-[(2S,5R)-4-(2,2-dimethylpropyl)-5-methyl-2-phenyl-piperazin-1-yl]acetamide (0.2 g, 630.07 μmol, 76.46% yield) that was used in the next step without purification.

LCMS(ESI): [M+1]⁺ m/z: calcd 317.4; found 318.2; Rt=0.748 min.

### Step 6: The synthesis of 2-oxo-2-[(2S,5R)-4-(2,2-dimethylpropyl)-5-methyl-2-phenyl-piperazin-1-yl]-N-(1-tetrahydropyran-2-ylpyrazolo[4,3-c]pyridin-7-yl)acetamide

2-oxo-2-[(2S,5R)-4-(2,2-dimethylpropyl)-5-methyl-2-phenyl-piperazin-1-yl]acetamide (0.141 g, 444.20 mol), 7-bromo-1-tetrahydropyran-2-yl-pyrazolo[4,3-c]pyridine (125.32 mg, 444.20 μmol), copper (28.23 mg, 444.20 μmol), iodocopper (84.60 mg, 444.20 μmol, 15.05 μL), cesium carbonate (289.46 mg, 888.40 mol) were mixed in the DXN (10 mL) under argon stream, and (1R,2R)—N₁,N₂-dimethylcyclohexane-1,2-diamine (94.78 mg, 666.30 mol) was added thereto. The reaction mixture was stirred at 100° C. for 12 hr.

After completion the reaction mixture was cooled to the 25° C., and filtered through thin pad of silica, the filter cake was washed by DXN (3*5 mL), and the filtrate was evaporated to dryness under reduced pressure to afford crude 2-oxo-2-[(2S,5R)-4-(2,2-dimethylpropyl)-5-methyl-2-phenyl-piperazin-1-yl]-N-(1-tetrahydropyran-2-ylpyrazolo[4,3-c]pyridin-7-yl)acetamide (0.86 g, 1.66 mmol, 373.29% yield), that was directly used in the next step without additional purification.

LCMS(ESI): [M+1]⁺ m/z: calcd 518.3; found 520.2; Rt=1.211 min.

### Step 7: The synthesis of 2-oxo-N-(1H-pyrazolo[4,3-c]pyridin-7-yl)-2-[(2S,5R)-4-(2,2-dimethylpropyl)-5-methyl-2-phenyl-piperazin-1-yl]acetamide

2-oxo-2-[(2S,5R)-4-(2,2-dimethylpropyl)-5-methyl-2-phenyl-piperazin-1-yl]-N-(1-tetrahydropyran-2-ylpyrazolo[4,3-c]pyridin-7-yl)acetamide (0.86 g, 1.66 mmol) was dissolved in the DXN/HCl (10 mL), and this reaction mixture was stirred at 25° C. for 12 hr. After completion the solvent was evaporated to dryness under reduced pressure, and 2-oxo-N-(1H-pyrazolo[4,3-c]pyridin-7-yl)-2-[(2S,5R)-4-(2,2-dimethylpropyl)-5-methyl-2-phenyl-piperazin-1-yl]acetamide (0.0339 g, 66.80 mol, 4.03% yield, 2HCl) (fraction 1: m=0.0216g.purity=96.36; fraction 2: m=0.0123 g., purity=98.9%) was obtained by reverse phase HPLC of the residue.

¹H NMR (600 MHz, dmso) 6 0.69-0.86 (m, 9H), 0.99-1.08 (m, 3H), 1.94-2.01 (m, 1H), 2.18-2.26 (m, 1H), 2.85-3.18 (m, 3H), 3.41-3.68 (m, 1H), 3.86-4.18 (m, 1H), 5.40-5.53 (m, 1H), 7.23-7.40 (m, 3H), 7.41-7.62 (m, 2H), 8.10-8.35 (m, 1H), 8.36-8.53 (m, 1H), 8.81-8.99 (m, 1H), 10.95-11.19 (m, 1H), 13.04 (s, 1H).

LCMS(ESI): [M+1]⁺ m/z: calcd. 434.2; found 435.2; Rt=2.472 min.

### Example 41. Compound 3 N-(6-amino-5-methylpyridin-3-yl)-2-((2S,5R)-2-(4-fluorophenyl)-5-methyl-4-pivaloylpiperazin-1-yl)-2-oxoacetamide

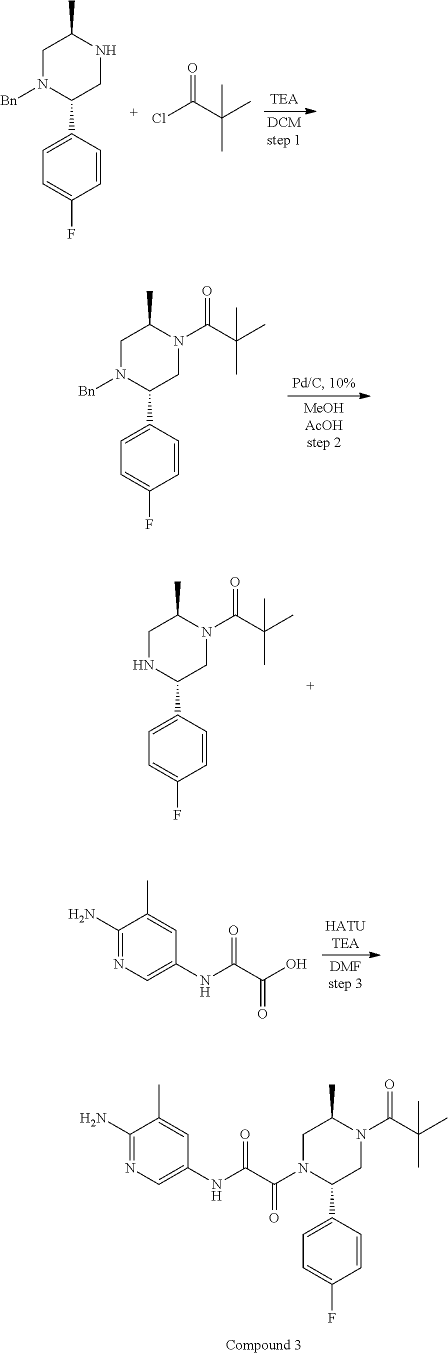

(2S,5R)-1-benzyl-2-(4-fluorophenyl)-5-methylpiperazine was prepared by the following. Borane dimethyl sulfide complex (2.66 g, 35.06 mmol, 3.33 mL) was added dropwise to the solution of (3S,6R)-4-benzoyl-3-(4-fluorophenyl)-6-methyl-piperazin-2-one (2.19 g, 7.01 mmol) in THF (30 mL). Resulting mixture was stirred at 65° C. for 18 hr. Then, it was cooled to rt and excess of borane was destroyed by dropwise addition of MeOH (10 ml). After H₂ evolution ceased, volatiles were removed under reduced pressure and residue was taken up in 2M aq. HCl (40 ml) and stirred at 50° C. for 40 minutes. Resulting cloudy solution was filtered and extracted with DCM (2×10 ml). DCM layers were discarded and aqueous layer was basified to pH≈11 with solid potassium hydroxide. Precipitated amine was extracted with DCM (2×25 ml). Organic layers were separated, dried over K₂CO₃ and concentrated in vacuum, affording (2S,5R)-1-benzyl-2-(4-fluorophenyl)-5-methyl-piperazine (1.31 g, 4.61 mmol, 65.70% yield).

LCMS(ESI): [M]⁺ m/z: calcd 284.2; found 285.2; Rt=1.049 min.

### Step 1: Synthesis of 1-((2R,5S)-4-benzyl-5-(4-fluorophenyl)-2-methylpiperazin-1-yl)-2,2-dimethylpropan-1-one

2,2-Dimethylpropanoyl chloride (508.82 mg, 4.22 mmol, 516.57 uL) was added dropwise to the solution of (2S,5R)-1-benzyl-2-(4-fluorophenyl)-5-methyl-piperazine (1 g, 3.52 mmol) and TEA (711.68 mg, 7.03 mmol, 980.27 uL) in DCM (30 mL). Resulting mixture was stirred at 20° C. for 2 hr. Then, 15% aq. K₂CO₃ solution (20 ml) was added and stirring was continued for 10 min. After that, organic layer was separated, dried over K₂CO₃ and concentrated under reduced pressure, affording 1-[(2R,5S)-4-benzyl-5-(4-fluorophenyl)-2-methyl-piperazin-1-yl]-2,2-dimethyl-propan-1-one (1.34 g, crude).

LCMS(ESI): [M]⁺ m/z: calcd 368.2; found 369.2; Rt=1.589 min.

### Step 2: Synthesis of 1-((2R,5S)-5-(4-fluorophenyl)-2-methylpiperazin-1-yl)-2,2-dimethylpropan-1-one

Palladium, 10% on carbon (350 mg, 328.89 umol, 10% purity) was added to the solution of 1-[(2R,5S)-4-benzyl-5-(4-fluorophenyl)-2-methyl-piperazin-1-yl]-2,2-dimethyl-propan-1-one (1.36 g, 3.68 mmol) in MeOH (30 mL) and acetic acid (10 mL). Reaction flask was evacuated and backfilled with hydrogen (111.39 mg, 55.26 mmol) from attached balloon. Resulting mixture was stirred at 50° C. for 16 hr. Then, catalyst was filtered off and filtrate was concentrated under reduced pressure. Residue was partitioned between 10% aq. K₂CO₃ solution (20 ml) and DCM (40 ml). Organic layer was separated, dried over K₂CO₃ and concentrated in vacuum, affording 1-[(2R,5S)-5-(4-fluorophenyl)-2-methyl-piperazin-1-yl]-2,2-dimethyl-propan-1-one (0.98 g, 3.52 mmol, 95.57% yield).

LCMS(ESI): [M]⁺ m/z: calcd 278.2; found 279.2; Rt=0.928 min.

### Step 3: Synthesis of N-(6-amino-5-methylpyridin-3-yl)-2-((2S,5R)-2-(4-fluorophenyl)-5-methyl-4-pivaloylpiperazin-1-yl)-2-oxoacetamide (Compound 3)

Prepared by Scheme 1.1, step 10A. Yield: 156 mg (47.66%).

HPLC conditions: Column: YMC Triart C18 100*20 mm, 5 microM; 0-5 min 50-80% MeOH/water+0.1% NH₄OH 40 ml/min; (loading pump 4 ml/min MeOH).

Compound 3: ¹H NMR (600 MHz, DMSO-d₆) δ (ppm) 1.08 (m, 9H), 1.23 (m, 3H), 2.02 (m, 3H), 3.20 (m, 2H), 3.82 (dd, 1H), 4.45 (m, 1H), 4.89 (m, 1H), 5.64 (m, 3H), 7.17 (m, 2H), 7.28 (m, 1H), 7.37 (m, 1H), 7.48 (m, 1H), 8.02 (m, 1H), 10.58 (m, 1H).

LCMS(ESI): [M]⁺ m/z: calcd 455.2; found 456.2; Rt=2.828 min.

### Example 42. Compound 148, Compound 146 and Compound 144 tert-butyl 4-(2-((6-amino-5-cyclopropylpyridin-3-yl)amino)-2-oxoacetyl)-5-(4-fluorophenyl)-2-methylpiperazine-1-carboxylate

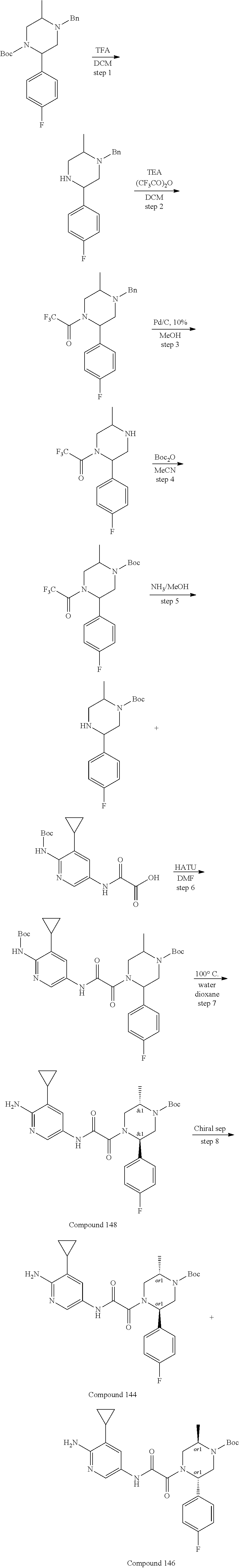

### Step 1: Synthesis of 1-benzyl-5-(4-fluorophenyl)-2-methylpiperazine

tert-butyl 4-benzyl-2-(4-fluorophenyl)-5-methyl-piperazine-1-carboxylate (3.04 g, 7.90 mmol) was dissolved in DCM (10 mL) and TFA (10 mL) was added thereto. The resulting mixture was stirred for 1 hr.

The reaction mixture was carefully poured into K₂CO₃ solution (25 g in 100 ml of water) and the resulting mixture was extracted with DCM (2*40 ml). Combined organic layers were dried over Na₂SO₄, filtered and concentrated in vacuo to obtain 1-benzyl-5-(4-fluorophenyl)-2-methyl-piperazine (2.23 g, 7.84 mmol, 99.28% yield) LCMS(ESI): [M]⁺ m/z: calcd 284.2; found 285.2; Rt=0.813 min.

### Step 2: Synthesis of 4-benzyl-2-(4-fluorophenyl)-5-methylpiperazin-1-yl)-2,2,2-trifluoroethanone

1-benzyl-5-(4-fluorophenyl)-2-methyl-piperazine (2.25 g, 7.91 mmol) was dissolved in DCM (50 mL) and Triethylamine (2.40 g, 23.74 mmol, 3.31 mL) was added thereto. The resulting mixture was cooled to −5° C. in an ice/methanol bath and (2,2,2-trifluoroacetyl) 2,2,2-trifluoroacetate (2.16 g, 10.29 mmol, 1.45 mL) was added dropwise to the reaction mixture. After addition completed, the reaction mixture was allowed to warm to room temperature and stirred overnight. Water (40 ml) was added to the reaction mixture and an organic layer was separated. The aqueous layer was extracted with DCM (50 ml) and combined organic layers were dried over Na₂SO₄, filtered and concentrated in vacuo. The residue was purified by column chromatography to obtain 1-[4-benzyl-2-(4-fluorophenyl)-5-methyl-piperazin-1-yl]-2,2,2-trifluoro-ethanone (1.95 g, 5.13 mmol, 64.79% yield).

LCMS(ESI): [M]⁺ m/z: calcd 380.2; found 381.2; Rt=1.468 min.

### Step 3: Synthesis of 2,2,2-trifluoro-1-(2-(4-fluorophenyl)-5-methylpiperazin-1-yl)ethanone

1-[4-benzyl-2-(4-fluorophenyl)-5-methyl-piperazin-1-yl]-2,2,2-trifluoro-ethanone (1.95 g, 5.13 mmol) was dissolved in MeOH (50 mL) and Palladium, 10% on carbon, Type 487, dry (436.45 mg, 4.10 mmol) was added thereto. The reaction vessel was evacuated and backfilled three times with hydrogen and the resulting mixture was hydrogenated at 1 atm (balloon) at room temperature overnight. The catalyst was filtered off and the filtrate was concentrated in vacuo to obtain 2,2,2-trifluoro-1-[2-(4-fluorophenyl)-5-methyl-piperazin-1-yl]ethanone (1.41 g, 4.85 mmol, 94.56% yield) LCMS(ESI): [M]⁺ m/z: calcd 290.2; found 291.2; Rt=0.724 min.

### Step 4: Synthesis of tert-butyl 5-(4-fluorophenyl)-2-methyl-4-(2,2,2-trifluoroacetyl)piperazine-1-carboxylate

2,2,2-Trifluoro-1-[2-(4-fluorophenyl)-5-methyl-piperazin-1-yl]ethanone (0.703 g, 2.42 mmol) was dissolved in MeCN (7 mL) and di-tert-butyl dicarbonate (528.59 mg, 2.42 mmol, 555.83 uL) was added thereto. The resulting mixture was stirred overnight. The reaction mixture was concentrated in vacuum to obtain tert-butyl 5-(4-fluorophenyl)-2-methyl-4-(2,2,2-trifluoroacetyl)piperazine-1-carboxylate (0.954 g, crude).

LCMS(ESI): [M-Boc]⁺ m/z: calcd 290.2; found 291.2; Rt=1.478 min.

### Step 5: Synthesis of tert-butyl 5-(4-fluorophenyl)-2-methylpiperazine-1-carboxylate

tert-Butyl 5-(4-fluorophenyl)-2-methyl-4-(2,2,2-trifluoroacetyl)piperazine-1-carboxylate (954 mg, 2.44 mmol) was dissolved in NH₃/MeOH (15 mL) and the resulting mixture was stirred overnight. The reaction mixture was concentrated in vacuum. Conversion rate was 56% by LCMS. The residue was dissolved in NH₃/MeOH (25 mL) and the resulting mixture was allowed to stir overnight. The reaction mixture was concentrated in vacuum. Conversion rate was 86% by LCMS. The residue was dissolved in NH₃/MeOH (25 mL) and the resulting mixture was allowed to stir overnight. The reaction mixture was concentrated in vacuum to obtain tert-butyl 5-(4-fluorophenyl)-2-methyl-piperazine-1-carboxylate (706 mg, 2.40 mmol, 98.14% yield).

LCMS(ESI): [M]⁺ m/z: calcd 294.2; found 295.2; Rt=1.013 min.

### Step 6: Synthesis of tert-butyl 4-(2-((6-((tert-butoxycarbonyl)amino)-5-cyclopropylpyridin-3-yl)amino)-2-oxoacetyl)-5-(4-fluorophenyl)-2-methylpiperazine-1-carboxylate

Prepared by Scheme 1.1, step 10A. Yield: 277.7 mg (45.59%).

HPLC conditions: Column:SunFire 100*19 mm, 5 microM; 2-10 min 60-75% MeOH/water 30 ml/min; (loading pump 4 ml/min MeOH).

LCMS(ESI): [M]⁺ m/z: calcd 597.2; found 598.2; Rt=1.378 min.

### Step 7: Synthesis of tert-butyl 4-(2-((6-amino-5-cyclopropylpyridin-3-yl)amino)-2-oxoacetyl)-5-(4-fluorophenyl)-2-methylpiperazine-1-carboxylate (Compound 148)

Prepared by Boc Group Deportection Method A. Yield: 43.6 mg (18.86%).

Compound 148: ¹H NMR (500 MHz, DMSO-d₆) δ (ppm) 0.38-0.48 (m, 2H), 0.82-0.91 (m, 2H), 1.09-1.17 (m, 3H), 1.30-1.38 (m, 9H), 1.59-1.70 (m, 1H), 2.73-3.07 (m, 1H), 3.36-3.52 (m, 1H), 3.54-3.96 (m, 1H), 4.02-4.22 (m, 1H), 4.30-4.43 (m, 1H), 5.20-5.56 (m, 1H), 5.72-5.80 (m, 2H), 7.16-7.25 (m, 2H), 7.26-7.41 (m, 3H), 7.91-8.07 (m, 1H), 10.32-10.63 (m, 1H).

LCMS(ESI): [M]⁺ m/z: calcd 497.2; found 498.2; Rt=2.638 min.

### Step 8: Chiral Separation (Compound 146 and Compound 144)

Racemic tert-butyl (2R,5S)-4-[2-[(6-amino-5-cyclopropyl-3-pyridyl)amino]-2-oxo-acetyl]-5-(4-fluorophenyl)-2-methyl-piperazine-1-carboxylate (29.8 mg, 59.89 mol) was chirally separated (Column: Chiralpak IC (250-20 mm-5 m); Mobile phase: Hexane-IPA-MeOH, 60-20-20 Flow Rate: 12 mL/min; Column Temperature: 22° C.; Wavelength: 205 nm, 215 nm to obtain tert-butyl (2S,5R)-4-[2-[(6-amino-5-cyclopropyl-3-pyridyl)amino]-2-oxo-acetyl]-5-(4-fluorophenyl)-2-methyl-piperazine-1-carboxylate (12.5 mg, 25.12 mol, 41.95% yield) (RT=14.83 min) and tert-butyl (2R,5S)-4-[2-[(6-amino-5-cyclopropyl-3-pyridyl)amino]-2-oxo-acetyl]-5-(4-fluorophenyl)-2-methyl-piperazine-1-carboxylate (12.97 mg, 26.07 mol, 43.52% yield) (RT=19.46 min).

Rel Time for Compound 146 in analytical conditions (Chiralcel OD-H (250*4.6, 5 mkm), Hexane-IPA-MeOH, 95-5-5, 0.6 ml/min as mobile phase) 30.48 min and for

Compound 144 33.08 min.

Compound 146: Retention time: 30.48 min

¹H NMR (500 MHz, DMSO-d₆) δ (ppm) 0.38-0.48 (m, 2H), 0.82-0.91 (m, 2H), 1.09-1.17 (m, 3H), 1.30-1.38 (m, 9H), 1.59-1.70 (m, 1H), 2.73-3.07 (m, 1H), 3.36-3.52 (m, 1H), 3.54-3.96 (m, 1H), 4.02-4.22 (m, 1H), 4.30-4.43 (m, 1H), 5.20-5.56 (m, 1H), 5.72-5.80 (m, 2H), 7.16-7.25 (m, 2H), 7.26-7.41 (m, 3H), 7.91-8.07 (m, 1H), 10.32-10.63 (m, 1H).

LCMS(ESI): [M]⁺ m/z: calcd 497.2; found 498.2; Rt=2.638 min.

Compound 144: Retention time: 33.08 min

¹H NMR (500 MHz, DMSO-d₆) δ (ppm) 0.38-0.48 (m, 2H), 0.82-0.91 (m, 2H), 1.09-1.17 (m, 3H), 1.30-1.38 (m, 9H), 1.59-1.70 (m, 1H), 2.73-3.07 (m, 1H), 3.36-3.52 (m, 1H), 3.54-3.96 (m, 1H), 4.02-4.22 (m, 1H), 4.30-4.43 (m, 1H), 5.20-5.56 (m, 1H), 5.72-5.80 (m, 2H), 7.16-7.25 (m, 2H), 7.26-7.41 (m, 3H), 7.91-8.07 (m, 1H), 10.32-10.63 (m, 1H).

LCMS(ESI): [M]⁺ m/z: calcd 497.2; found 498.2; Rt=2.638 min.

### Example 43. Compound 54 N-(4-amino-1H-pyrazolo[4,3-c]pyridin-7-yl)-2-((2S,5R)-4-(3-(dimethylamino)-2,2-dimethylpropanoyl)-5-methyl-2-phenylpiperazin-1-yl)-2-oxoacetamide

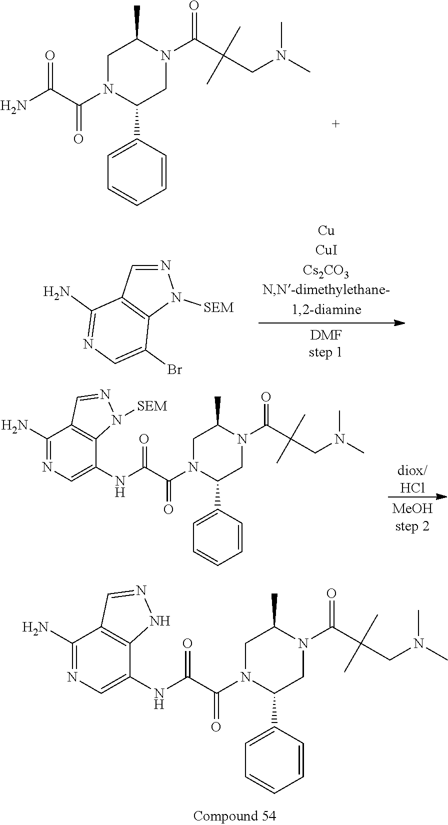

The synthesis of 2-[(2S,5R)-4-[3-(dimethylamino)-2,2-dimethyl-propanoyl]-5-methyl-2-phenyl-piperazin-1-yl]-2-oxo-acetamide is given by the following. 2,2,2-Trifluoroethyl 2-[(2S,5R)-4-[3-(dimethylamino)-2,2-dimethyl-propanoyl]-5-methyl-2-phenyl-piperazin-1-yl]-2-oxo-acetate (0.3 g, 655.76 mol) was dissolved in NH₃/MeOH (10 mL) and the resulting mixture was left to stir at 25° C. for 16 hr. Upon completion of the reaction, solvent was evaporated to dryness to obtain 2-[(2S,5R)-4-[3-(dimethylamino)-2,2-dimethyl-propanoyl]-5-methyl-2-phenyl-piperazin-1-yl]-2-oxo-acetamide (0.3 g, crude).

LCMS(ESI): [M]⁺ m/z: calcd 374.2; found 375.2; Rt=0.761 min.

### Step 1: Synthesis of N-(4-amino-1-((2-(trimethylsilyl)ethoxy)methyl)-1H-pyrazolo[4,3-c]pyridin-7-yl)-2-((2S,5R)-4-(3-(dimethylamino)-2,2-dimethylpropanoyl)-5-methyl-2-phenylpiperazin-1-yl)-2-oxoacetamide

2-Oxo-2-[(2S,5R)-4-[3-(dimethylamino)-2,2-dimethyl-propanoyl]-5-methyl-2-phenyl-piperazin-1-yl]acetamide (0.2 g, 534.08 mol), 7-bromo-1-(2-trimethylsilylethoxymethyl)pyrazolo[4,3-c]pyridin-4-amine (183.35 mg, 534.08 mol), Cu (33.94 mg, 534.08 mol), cesium carbonate (348.03 mg, 1.07 mmol), CuI (101.72 mg, 534.08 μmol, 18.10 L) and (1S,2S)—N,N′-bis-methyl-1,2-cyclohexane-diamine (113.95 mg, 801.12 μmol, 126.33 L) were mixed in DMF (4 mL) under argon. The resulting mixture was allowed to stir at 100° C. for 15 hr in vial. Upon completion of the reaction, dioxane was evaporated and residue was subjected by HPLC (column: XBridge C18 100×19 mm, 5 um; mobile phase: 20-65% 0-6 min H₂O/MeOH/0.1% NH₄OH, flow rate: 30 ml/min (loading pump 4 ml/min MeOH), affording N-[4-amino-1-(2-trimethylsilylethoxymethyl)pyrazolo[4,3-c]pyridin-7-yl]-2-oxo-2-[(2S,5R)-4-[3-(dimethylamino)-2,2-dimethyl-propanoyl]-5-methyl-2-phenyl-piperazin-1-yl]acetamide (90.6 mg, 142.26 μmol, 26.64% yield) on two portions.

LCMS(ESI): [M]⁺ m/z: calcd 636.2; found 637.2; Rt=1.958 min.

### Step 2: Synthesis of N-(4-amino-1H-pyrazolo[4,3-c]pyridin-7-yl)-2-((2S,5R)-4-(3-(dimethylamino)-2,2-dimethylpropanoyl)-5-methyl-2-phenylpiperazin-1-yl)-2-oxoacetamide (Compound 54)

N-[4-Amino-1-(2-trimethylsilylethoxymethyl)pyrazolo[4,3-c]pyridin-7-yl]-2-oxo-2-[(2S,5R)-4-[3-(dimethylamino)-2,2-dimethyl-propanoyl]-5-methyl-2-phenyl-piperazin-1-yl]acetamide (90.6 mg, 142.26 μmol) was dissolved in a mixture of diox/HCl (2 mL) and MeOH (2 mL), the resulting mixture was stirred at 25° C. for 16 hr. After that time, solvent was evaporated to dryness to afford crude product. Crude product was subjected by HPLC (column: XBridge C18 100×19 mm, 5 um; mobile phase: 20-65% 0-6min H₂O/MeOH/0.1% NH₄OH, flow rate: 30 ml/min (loading pump 4 ml/min MeOH), affording N-(4-amino-1H-pyrazolo[4,3-c]pyridin-7-yl)-2-oxo-2-[(2S,5R)-4-[3-(dimethylamino)-2,2-dimethyl-propanoyl]-5-methyl-2-phenyl-piperazin-1-yl]acetamide (42.4 mg, 73.16 mol, 51.43% yield, 2HCl).

Compound 54: ¹H NMR (600 MHz, DMSO-d₆) δ (ppm) 0.92-1.57 (m, 1OH), 2.72 (s, 6H), 2.86-3.05 (m, 2H), 3.44-6.51 (m, 5H), 7.02-8.02 (m, 8H), 8.21-9.98 (m, 2H), 10.80-11.14 (m, 1H), 12.85-13.98 (m, 1H).

LCMS(ESI): [M]⁺ m/z: calcd 506.2; found 507.2; Rt=1.366 min.

### Example 44. Compound 11 N-(6-amino-5-methylpyridin-3-yl)-2-((2S,5R)-4-isobutyryl-5-methyl-2-(4-(4-methylpiperazin-1-yl)phenyl)piperazin-1-yl)-2-oxoacetamide

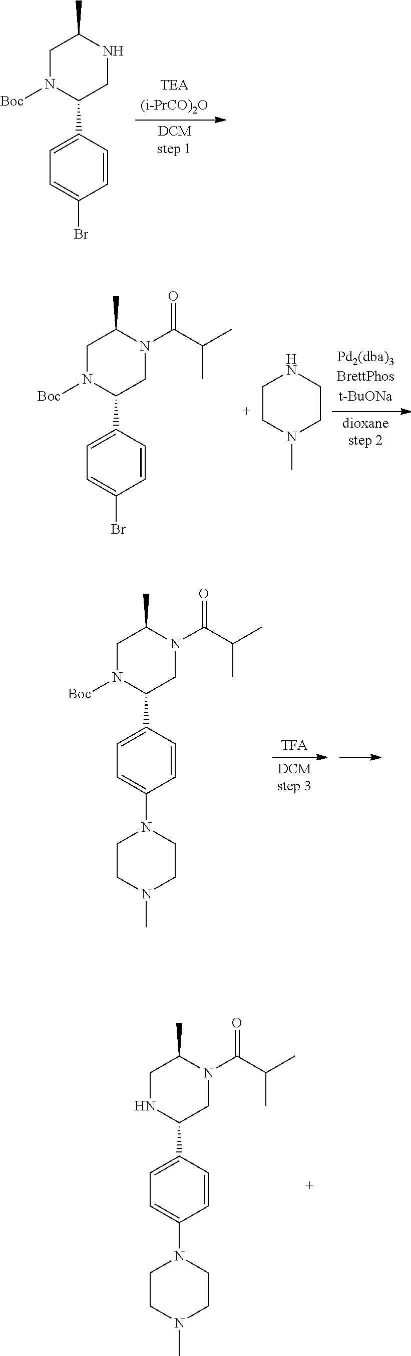

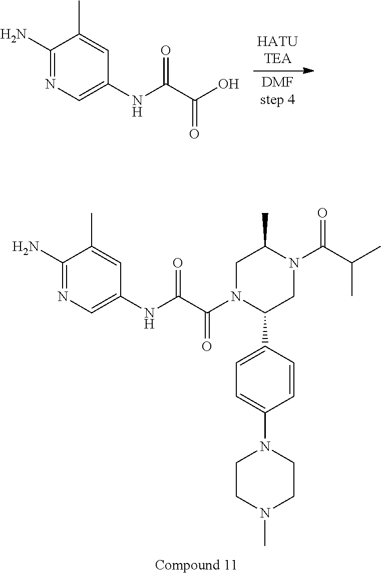 [SMILES: Cc1cc(NC(=O)C(=O)O)cnc1N ; Cc1cc(NC(=O)C(=O)N2C[C@@H](C)N(C(=O)C(C)C)C[C@@H]2c2ccc(N3CCN(C)CC3)cc2)cnc1N]

The synthesis of (2S,5R)-1-benzyl-2-(4-fluorophenyl)-5-methylpiperazine is given by the following procedure. Borane dimethyl sulfide complex (2.66 g, 35.06 mmol, 3.33 mL) was added dropwise to the solution of (3S,6R)-4-benzoyl-3-(4-fluorophenyl)-6-methyl-piperazin-2-one (2.19 g, 7.01 mmol) in THF (30 mL). Resulting mixture was stirred at 65° C. for 18 hr. Then, it was cooled to rt and excess of borane was destroyed by dropwise addition of MeOH (10 ml). After H₂ evolution ceased, volatiles were removed under reduced pressure and residue was taken up in 2M aq. HCl (40 ml) and stirred at 50° C. for 40 minutes. Resulting cloudy solution was filtered and extracted with DCM (2×10 ml). DCM layers were discarded and aqueous layer was basified to pH≈11 with solid potassium hydroxide. Precipitated amine was extracted with DCM (2×25 ml). Organic layers were separated, dried over K₂CO₃ and concentrated in vacuum, affording (2S,5R)-1-benzyl-2-(4-fluorophenyl)-5-methyl-piperazine (1.31 g, 4.61 mmol, 65.70% yield).

LCMS(ESI): [M]⁺ m/z: calcd 284.2; found 285.2; Rt=1.049 min.

### Step 1: Synthesis of (2S,5R)-tert-butyl 2-(4-bromophenyl)-4-isobutyryl-5-methylpiperazine-1-carboxylate

tert-Butyl (2S,5R)-2-(4-bromophenyl)-5-methyl-piperazine-1-carboxylate (0.75 g, 2.11 mmol) and TEA (640.86 mg, 6.33 mmol, 882.73 L) were mixed together in DCM (15 mL) and the resulting solution was cooled to 0° C. in an ice/MeOH bath. 2-Methylpropanoyl 2-methylpropanoate (333.96 mg, 2.11 mmol, 350.06 L) was added dropwise to the previous solution and the resulting mixture was allowed to warm to rt and stirred overnight. The reaction mixture was diluted with DCM and the resulting solution was washed with water, dried over Na₂SO₄, filtered and evaporated to obtain tert-butyl (2S,5R)-2-(4-bromophenyl)-5-methyl-4-(2-methylpropanoyl)piperazine-1-carboxylate (0.8 g, 1.88 mmol, 89.09% yield) which was used in the next step without further purification.

LCMS(ESI): [M-t-Bu]⁺ m/z: calcd 369.2; found 370.2; Rt=1.574 min.

### Step 2: Synthesis of (2S,5R)-tert-butyl 4-isobutyryl-5-methyl-2-(4-(4-methylpiperazin-1-yl)phenyl)piperazine-1-carboxylate

tert-Butyl (2S,5R)-2-(4-bromophenyl)-5-methyl-4-(2-methylpropanoyl)piperazine-1-carboxylate (0.8 g, 1.88 mmol), 1-methylpiperazine (188.38 mg, 1.88 mmol, 208.62 μL), sodium tert-butoxide (361.48 mg, 3.76 mmol) and 2-(dicyclohexylphosphino)_{3,6}-dimethoxy-2′,4′,6′-triisopropyl-1,1′-biphenyl (100.95 mg, 188.08 μmol) were mixed together in dioxane (20 mL) and the resulting mixture was evacuated and backfilled three times with argon. tris(Dibenzylideneacetone)dipalladium (O) (86.11 mg, 94.04 mol) was added to the previous mixture and the resulting mixture was heated at 100° C. (oil bath) overnight. The reaction mixture was cooled and filtered, the filtrate was concentrated in vacuum to give crude product which was purified by FCC (MeOH in MTBE from 0% to 100%) to give tert-butyl (2S,5R)-5-methyl-2-[4-(4-methylpiperazin-1-yl)phenyl]-4-(2-methylpropanoyl)piperazine-1-carboxylate (0.6 g, 1.35 mmol, 71.75% yield).

LCMS(ESI): [M]⁺ m/z: calcd 444.2; found 445.2; Rt=0.980 min.

### Step 3: Synthesis of 2-methyl-1-((2R,5S)-2-methyl-5-(4-(4-methylpiperazin-1-yl)phenyl)piperazin-1-yl)propan-1-one

tert-Butyl (2S,5R)-5-methyl-2-[4-(4-methylpiperazin-1-yl)phenyl]-4-(2-methylpropanoyl)piperazine-1-carboxylate (0.6 g, 1.35 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain 2-methyl-1-[(2R,5S)-2-methyl-5-[4-(4-methylpiperazin-1-yl)phenyl]piperazin-1-yl]propan-1-one (0.2 g, 580.56 μmol, 43.02% yield) which was used in the next step without further purification.

LCMS(ESI): [M]⁺ m/z: calcd 344.2; found 345.2; Rt=0.171 min.

### Step 4: Synthesis of N-(6-amino-5-methylpyridin-3-yl)-2-((2S,5R)-4-isobutyryl-5-methyl-2-(4-(4-methylpiperazin-1-yl)phenyl)piperazin-1-yl)-2-oxoacetamide (Compound 11)

Prepared by Scheme 1.1 step 10A. Yield: 18 mg (18.29%).

HPLC conditions: Column: SunFire C18 100*19 mm, 5 microM; 2-10 min 40-55% MeOH+NH₃ 30 ml/min; (loading pump 4 ml/min MeOH).

Compound 11: ¹H NMR (600 MHz, DMSO-d₆) δ (ppm) 0.83 (d, 1H), 0.90 (t, 2H), 0.93 (t, 1H), 0.99 (dd, 2H), 1.08 (d, 1H), 1.23 (dd, 2H), 1.98-2.03 (m, 3H), 2.17-2.20 (m, 3H), 2.38-2.41 (m, 4H), 2.69-3.20 (m, 7H), 3.55-4.92 (m, 3H), 5.16-5.66 (m, 3H), 6.82-6.94 (m, 2H), 7.05-7.19 (m, 2H), 7.28-7.53 (m, 1H), 7.88-8.05 (m, 1H), 10.34-10.61 (m, 1H).

LCMS(ESI): [M]⁺ m/z: calcd 521.2; found 522.2; Rt=1.724 min.

### Example 45. Compound 145 (2R,5S)-tert-butyl 4-(2-((6-amino-5-ethylpyridin-3-yl)amino)-2-oxoacetyl)-5-(4-fluorophenyl)-2-methylpiperazine-1-carboxylate

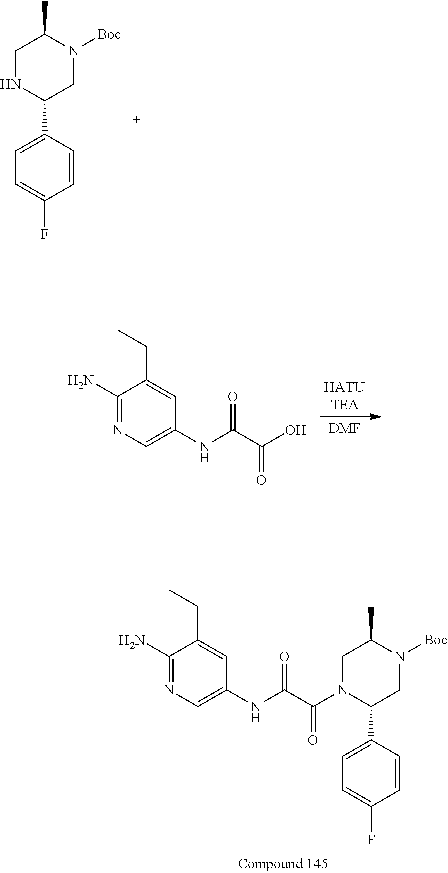

(2R,5S)-tert-butyl 5-(4-fluorophenyl)-2-methylpiperazine-1-carboxylatate is prepared by the following procedure. tert-Butyl (2R,5S)-5-(4-fluorophenyl)-2-methyl-4-(2,2,2-trifluoroacetyl)piperazine-1-carboxylate (355 mg, 909.39 μmol) was dissolved in NH₃/MeOH (10 mL) and the resulting mixture was stirred overnight. The reaction mixture was concentrated in vacuum to obtain tert-butyl (2R,5S)-5-(4-fluorophenyl)-2-methyl-piperazine-1-carboxylate (220 mg, 747.38 mol, 82.18% yield).

LCMS(ESI): [M-]⁺ m/z: calcd 294.2; found 295.2; Rt=1.064 min.

Prepared by Scheme 1.1 step 10A. Yield: 16.3 mg (19.76%).

HPLC conditions: Column: SunFire C18 100*19 mm, 5 microM; 2-10 min 30-45% water-MeOH 30 ml/min; (loading pump 4 ml/min MeOH).

Compound 145: ¹H NMR (600 MHz, DMSO-d₆) δ (ppm) 1.07-1.16 (m, 6H), 1.32-1.38 (m, 9H), 2.37-2.43 (m, 2H), 2.78-3.26 (m, 1H), 3.36-3.52 (m, 1H), 3.55-3.98 (m, 1H), 4.02-4.22 (m, 1H), 4.32-4.44 (m, 1H), 5.22-5.55 (m, 1H), 5.60-5.68 (m, 2H), 7.16-7.27 (m, 2H), 7.31-7.35 (m, 1H), 7.36-7.53 (m, 2H), 7.94-8.08 (m, 1H), 10.46-10.59 (m, 1H).

LCMS(ESI): [M]⁺ m/z: calcd 485.2; found 486.2; Rt=2.804 min.

### Example 46. Compound 117 N-(6-amino-5-methylpyridin-3-yl)-2-((2S,5R)-4-isobutyryl-5-methyl-2-(3-(4-methylpiperazin-1-yl)phenyl)piperazin-1-yl)-2-oxoacetamide

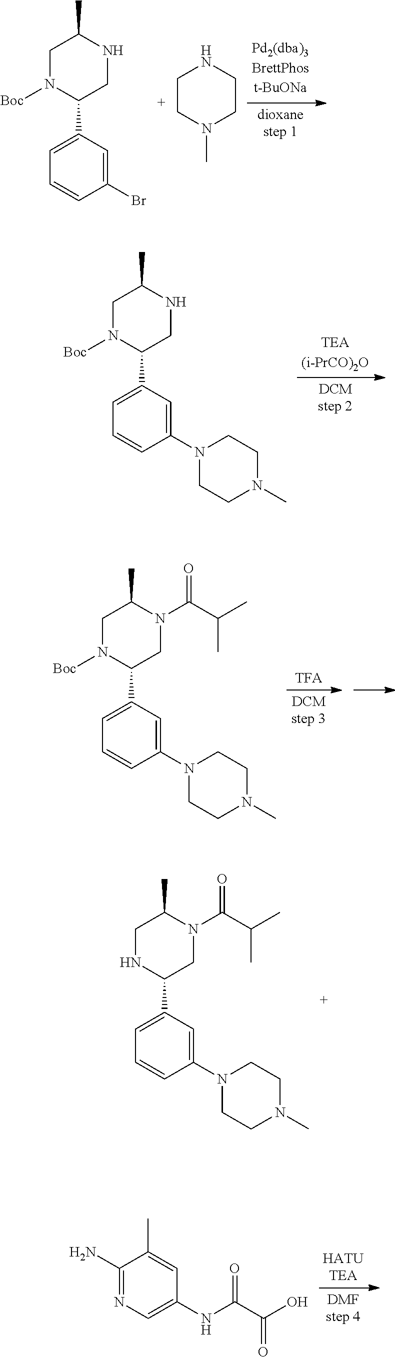

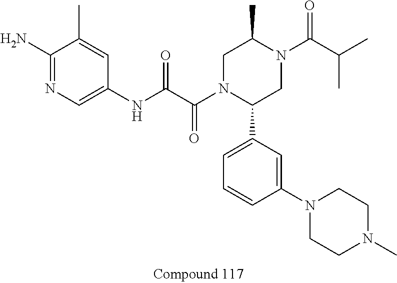 [SMILES: Cc1cc(NC(=O)C(=O)N2C[C@@H](C)N(C(=O)C(C)C)C[C@@H]2c2cccc(N3CCN(C)CC3)c2)cnc1N]

(2S,5R)-tert-butyl 2-(3-bromophenyl)-5-methylpiperazine-1-carboxylate is prepared by Scheme 1.4 step 6. Yield: 1.7 g of crude. LCMS(ESI): [M]⁺ m/z: calcd 355.2; found 356.2; Rt=1.086 min.

### Step 1: Synthesis of (2S,5R)-tert-butyl 5-methyl-2-(3-(4-methylpiperazin-1-yl)phenyl)piperazine-1-carboxylate

tert-Butyl (2S,5R)-2-(3-bromophenyl)-5-methyl-piperazine-1-carboxylate (226.00 mg, 636.14 μmol), 1-methylpiperazine (70.09 mg, 699.75 mol, 77.62 L) and sodium tert-butoxide (91.70 mg, 954.21 μmol) were mixed together in dioxane (5.00 mL) and 4,5-bis(diphenylphosphino)-9,9-dimethylxanthene (36.81 mg, 63.61 μmol) was added thereto. The resulting mixture was evacuated and backfilled three times with argon. tris(Dibenzylideneacetone)dipalladium (O) (29.13 mg, 31.81 μmol) was added to the previous mixture and the resulting mixture was heated at 100° C. (oil bath) for 15 hr. The reaction mixture was cooled and diluted with water (20 ml). The resulting mixture was extracted with EtOAc (2*20 ml). Combined organic layers were washed with brine (20 ml), dried over Na₂SO₄, filtered and evaporated to obtain crude material that was purified by FCC to obtain tert-butyl (2S,5R)-5-methyl-2-[3-(4-methylpiperazin-1-yl)phenyl]piperazine-1-carboxylate (226 mg, crude).

LCMS(ESI): [M-Boc]⁺ m/z: calcd 274.2; found 275.2; Rt=0.568 min.

### Step 2: Synthesis of (2S,5R)-tert-butyl 4-isobutyryl-5-methyl-2-(3-(4-methylpiperazin-1-yl)phenyl)piperazine-1-carboxylate

tert-Butyl (2S,5R)-5-methyl-2-[3-(4-methylpiperazin-1-yl)phenyl]piperazine-1-carboxylate (260.00 mg, 694.22 mol) and TEA (280.99 mg, 2.78 mmol, 387.04 L) were mixed together in DCM (5.10 mL) and the resulting solution was cooled to 0° C. in an ice/MeOH bath. 2-Methylpropanoyl 2-methylpropanoate (120.80 mg, 763.65 mol, 126.63 L) was added dropwise to the previous solution and the resulting mixture was allowed to warm to rt and stirred overnight. The reaction mixture was diluted with DCM (25 ml) and the resulting solution was washed with water (2*20 ml), dried over Na₂SO₄, filtered and evaporated to obtain tert-butyl (2S,5R)-5-methyl-2-[3-(4-methylpiperazin-1-yl)phenyl]-4-(2-methylpropanoyl)piperazine-1-carboxylate (315 mg, crude).

LCMS(ESI): [M]⁺ m/z: calcd 444.2; found 445.2; Rt=0.988 min.

### Step 3: Synthesis of 2-methyl-1-((2R,5S)-2-methyl-5-(3-(4-methylpiperazin-1-yl)phenyl)piperazin-1-yl)propan-1-one

tert-Butyl (2,5SR)-5-methyl-2-[3-(4-methylpiperazin-1-yl)phenyl]-4-(2-methylpropanoyl)piperazine-1-carboxylate (315.00 mg, 708.49 mol) was dissolved in DCM (2 mL),TFA (2 mL) was added in one portion. The resulting mixture was allowed to stir at 20° C. for 1.5 hr. Upon completion, DCM was evaporated, 20 ml of water was added to the residue and mixture was basified with K₂CO₃ to alkaline pH. Aqueous phase was extracted with DCM (3*15 ml), combined organic layers were dried over Na₂SO₄, filtered and concentrated under reduced pressure to afford 2-methyl-1-[(2R,5S)-2-methyl-5-[3-(4-methylpiperazin-1-yl)phenyl]piperazin-1-yl]propan-1-one (180 mg, crude).

LCMS(ESI): [M]⁺ m/z: calcd 344.2; found 345.2; Rt=0.609 min.

### Step 4: Synthesis of N-(6-amino-5-methylpyridin-3-yl)-2-((2S,5R)-4-isobutyryl-5-methyl-2-(3-(4-methylpiperazin-1-yl)phenyl)piperazin-1-yl)-2-oxoacetamide (Compound 117)

Prepared by Scheme 1.1 step 10A. Yield: 4.8 mg (2.26%).

HPLC conditions: Column: SunFire C18 100*19 mm, 5 microM; 2-10 min 40-65% MeCN+FA 30 ml/min; (loading pump 4 ml/min MeCN).

Compound 117: ¹H NMR (DMSO-d₆, 600 MHz): δ (ppm) 0.90 (m, 6H), 1.18 (m, 3H), 2.03 (m, 3H), 2.19 (m, 3H), 2.41 (m, 4H), 2.79 (m, 1H), 3.07 (m, 4H), 3.61 (m, 2H), 4.26 (m, 2H), 5.09 (m, 1H), 5.64 (m, 2H), 6.77 (m, 3H), 7.16 (m, 1H), 7.50 (m, 1H), 8.09 (m, 1H), 9.14 (m, 1H), 10.60 (m, 1H)

LCMS(ESI): [M]⁺ m/z: calcd 521.2; found 522.2; Rt=1.926 min.

### Example 47. Compound 67 N-(6-amino-5-ethylpyridin-3-yl)-2-((2S,5R)-2-(3-(2-(dimethylamino)ethoxy)phenyl)-4-isobutyryl-5-methylpiperazin-1-yl)-2-oxoacetamide

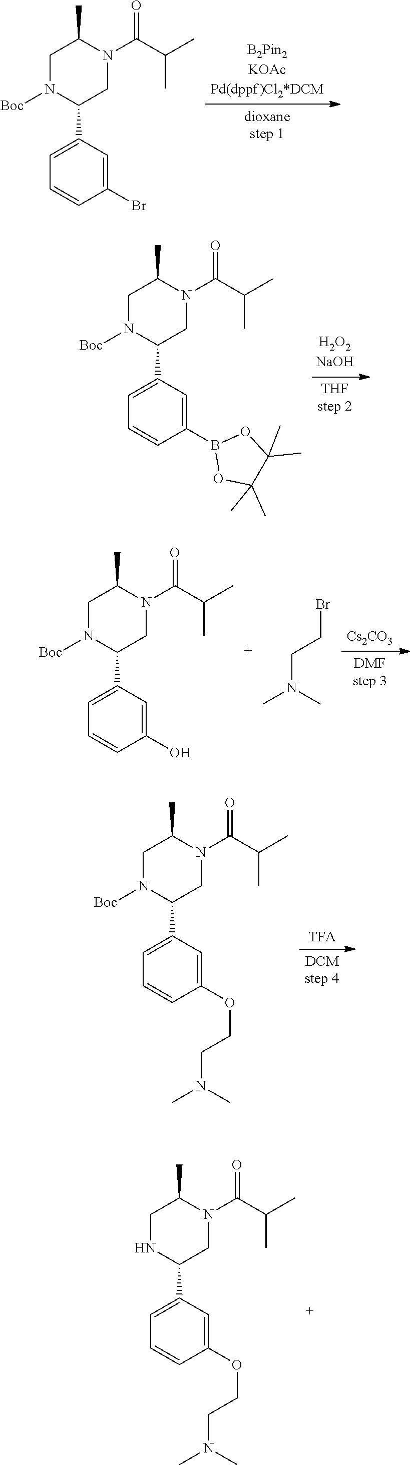

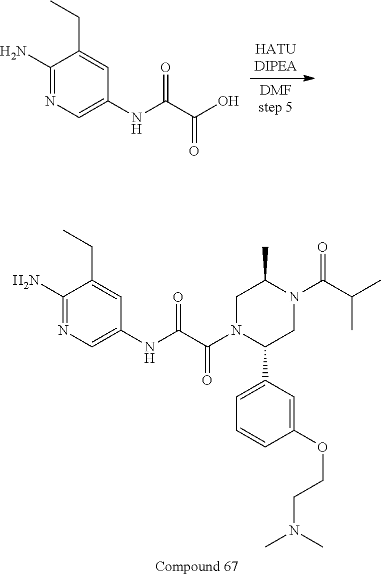 [SMILES: CCc1cc(NC(=O)C(=O)O)cnc1N ; "DMFstep 5" ; CCc1cc(NC(=O)C(=O)N2C[C@@H](C)N(C(=O)C(C)C)C[C@@H]2c2cccc(OCCN(C)C)c2)cnc1N]

(2S,5R)-tert-butyl 4-isobutyryl-5-methyl-2-(3-(4-methylpiperazin-1-yl)phenyl)piperazine-1-carboxylate is prepared by the following procedure. tert-Butyl (2S,5R)-5-methyl-2-[3-(4-methylpiperazin-1-yl)phenyl]piperazine-1-carboxylate (260.00 mg, 694.22 mol) and TEA (280.99 mg, 2.78 mmol, 387.04 L) were mixed together in DCM (5.10 mL) and the resulting solution was cooled to 0° C. in an ice/MeOH bath. 2-Methylpropanoyl 2-methylpropanoate (120.80 mg, 763.65 mol, 126.63 L) was added dropwise to the previous solution and the resulting mixture was allowed to warm to rt and stirred overnight. The reaction mixture was diluted with DCM (25 ml) and the resulting solution was washed with water (2*20 ml), dried over Na₂SO₄, filtered and evaporated to obtain tert-butyl (2S,5R)-5-methyl-2-[3-(4-methylpiperazin-1-yl)phenyl]-4-(2-methylpropanoyl)piperazine-1-carboxylate (315 mg, crude).

LCMS(ESI): [M]⁺ m/z: calcd 444.2; found 445.2; Rt=0.988 min.

### Step 1: Synthesis of (2S,5R)-tert-butyl 4-isobutyryl-5-methyl-2-(3-(4,4,5,5-tetramethyl-1,3,2-dioxaborolan-2-yl)phenyl)piperazine-1-carboxylate

To a stirred solution of tert-butyl (2S,5R)-2-(3-bromophenyl)-5-methyl-4-(2-methylpropanoyl)piperazine-1-carboxylate (1.7 g, 4.00 mmol) and 4,4,5,5-tetramethyl-2-(tetramethyl-1,3,2-dioxaborolan-2-yl)-1,3,2-dioxaborolane (1.07 g, 4.20 mmol) in dioxane (1.45 mL) was added potassium acetate (980.60 mg, 9.99 mmol, 624.58 L). The resulting suspension was degassed with argon. Pd(dppf)Cl₂*DCM (163.06 mg, 199.83 mol) was added. The reaction mixture was stirred at 90° C. for 16 hr. Upon completion, the reaction mixture was filtered and the filtrate was evaporated in vacuum to get an oily residue. Then compound was extracted with water/DCM (100 ml/200 ml). The desired product tert-butyl (2S,5R)-5-methyl-4-(2-methylpropanoyl)-2-[3-(4,4,5,5-tetramethyl-1,3,2-dioxaborolan-2-yl)phenyl]piperazine-1-carboxylate (1.67 g, 3.53 mmol, 88.45% yield) was isolated.

LCMS(ESI): [M]⁺ m/z: calcd 472.2; found 473.2; Rt=1.476 min.

### Step 2: Synthesis of (2S,5R)-tert-butyl 2-(3-hydroxyphenyl)-4-isobutyryl-5-methylpiperazine-1-carboxylate

To a stirred solution of tert-butyl (2S,5R)-5-methyl-4-(2-methylpropanoyl)-2-[3-(4,4,5,5-tetramethyl-1,3,2-dioxaborolan-2-yl)phenyl]piperazine-1-carboxylate (1.6 g, 3.39 mmol) in THF (50 mL) were added hydrogen peroxide 35% (115.20 mg, 3.39 mmol, 104.73 μL). After 1 hr sodium hydroxide, pearl (135.46 mg, 3.39 mmol, 63.60 L) was added. The resulting reaction mixture was stirred at 25° C. for 123 hr. Upon completion, 150 ml of water was added. Then MnO2 was added until reaction stopped. Then reaction mixture was filtered, 150 ml of MTBE was added to the filtrate and extracted. Organic phase was dried over Na₂SO₄ and evaporated. The desired product tert-butyl (2S,5R)-2-(3-hydroxyphenyl)-5-methyl-4-(2-methylpropanoyl)piperazine-1-carboxylate (740 mg, 2.04 mmol, 60.28% yield) was isolated as brown color state.

LCMS(ESI): [M]⁺ m/z: calcd 362.2; found 363.2; Rt=1.323 min.

### Step 3: Synthesis of (2S,5R)-tert-butyl 2-(3-(2-(dimethylamino)ethoxy)phenyl)-4-isobutyryl-5-methylpiperazine-1-carboxylate

To a stirred solution of tert-butyl (2S,5R)-2-(3-hydroxyphenyl)-5-methyl-4-(2-methylpropanoyl)piperazine-1-carboxylate (740 mg, 2.04 mmol) were added (2-bromoethyl)dimethylamine hydrobromide (2.38 g, 10.21 mmol) and cesium carbonate (6.65 g, 20.42 mmol) respectively at 80° C. The resulting reaction mixture was stirred at 80° C. for 24 hr. Upon completion, the reaction mixture was quenched with water 50 mL. The aqueous phase was extracted with MTBE 50 mL twice. The combined organic phase was washed with water 20 mL, brine 20 mL, dried over Na₂SO₄ and concentrated under reduced pressure to obtain crude product as brown color state. The obtained crude product was purified by reverse phase HPLC (Device (Mobile Phase, Column): SYSTEM 45-60% 0-1-6 min H₂O/MeCN/0.1% NH₄OH, flow: 30 ml/min (loading pump 4 ml/min MeOH); column: XBridge BEH C18 5 μm 130A) to afford product tert-butyl (2S,5R)-2-[3-[2-(dimethylamino)ethoxy]phenyl]-5-methyl-4-(2-methylpropanoyl)piperazine-1-carboxylate (87 mg, 200.65 mol, 9.83% yield). The desired product tert-butyl (2S,5R)-2-[3-[2-(dimethylamino)ethoxy]phenyl]-5-methyl-4-(2-methylpropanoyl)piperazine-1-carboxylate (87 mg, 200.65 mol, 9.83% yield) was isolated as a brown color state.

LCMS(ESI): [M]⁺ m/z: calcd 433.2; found 434.2; Rt=2.930 min.

### Step 4: Synthesis of 1-((2R,5S)-5-(3-(2-(dimethylamino)ethoxy)phenyl)-2-methylpiperazin-1-yl)-2-methylpropan-1-one

To a stirred solution of tert-butyl (2S,5R)-2-[3-[2-(dimethylamino)ethoxy]phenyl]-5-methyl-4-(2-methylpropanoyl)piperazine-1-carboxylate (87 mg, 200.65 mol) in DCM (5 mL) were added TFA (1.14 g, 10.03 mmol, 772.94 L) respectively at 25° C. The resulting reaction mixture was stirred at 25° C. for 12 hr. Upon completion, the reaction mixture was concentrated under reduced pressure. Then compound was dissolved in mixture DCM (30 ml)/NaHCO₃ solution (20 ml), extracted, dried over Na₂SO₄ and evaporated. The desired product 2-methyl-1-[(2R,5S)-5-[3-[2-(dimethylamino)ethoxy]phenyl]-2-methyl-piperazin-1-yl]propan-1-one (40 mg, 119.95 mol, 59.78% yield) was isolated as a brown color state.

LCMS(ESI): [M]⁺ m/z: calcd 333.2; found 334.2; Rt=0.392 min.

### Step 5: Synthesis of N-(6-amino-5-ethylpyridin-3-yl)-2-((2S,5R)-2-(3-(2-(dimethylamino)ethoxy)phenyl)-4-isobutyryl-5-methylpiperazin-1-yl)-2-oxoacetamide (Compound 67)

Prepared by Scheme 1.1, step 10B. Yield: 12 mg (19.07%).

HPLC conditions: Column: YMC Triart C18 100*20 mm, 5 microM; 0-5 min 10-45% water-MeCN+0.1% NH₄OH 30 ml/min; (loading pump 4 ml/min MeCN).

Compound 67: ¹H NMR (600 MHz, DMSO-d₆) δ (ppm) 0.98 (m, 12H), 2.19 (m, 6H), 2.39 (m, 4H), 2.60 (m, 2H), 2.77 (m, 1H), 3.17 (m, 1H), 3.65 (m, 1H), 3.99 (m, 2H), 4.49 (m, 1H), 5.56 (m, 3H), 6.86 (m, 3H), 7.25 (m, 1H), 7.51 (m, 1H), 7.97 (m, 1H), 10.60 (m, 1H).

LCMS(ESI): [M]⁺ m/z: calcd 524.2; found 525.2; Rt=1.963 min.

### Example 48. Compound 26, Compound 84, Compound 48 and Compound 15 N-(6-amino-5-cyclopropylpyridin-3-yl)-2-(2-(4-fluorophenyl)-4-isobutyl-5-methylpiperazin-1-yl)-2-oxoacetamide

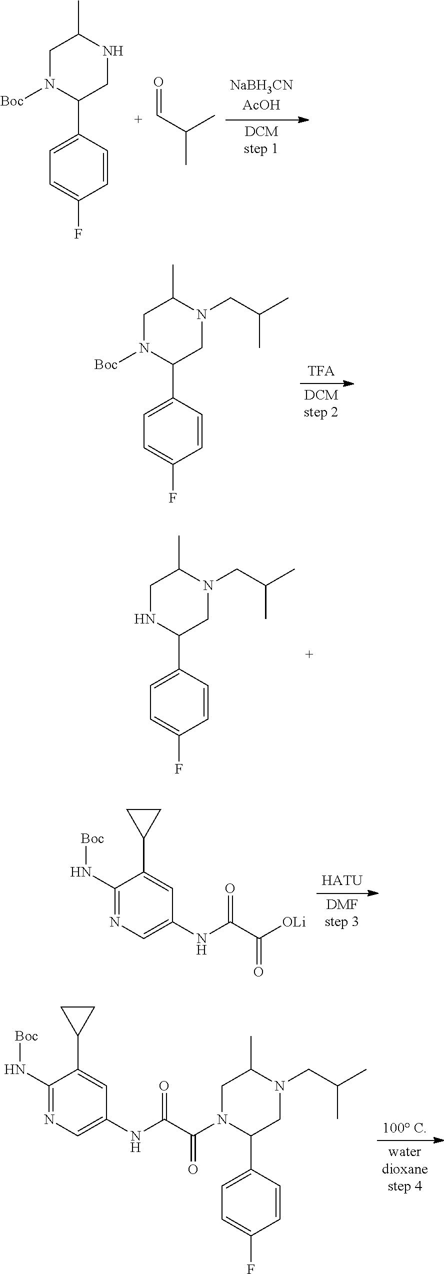

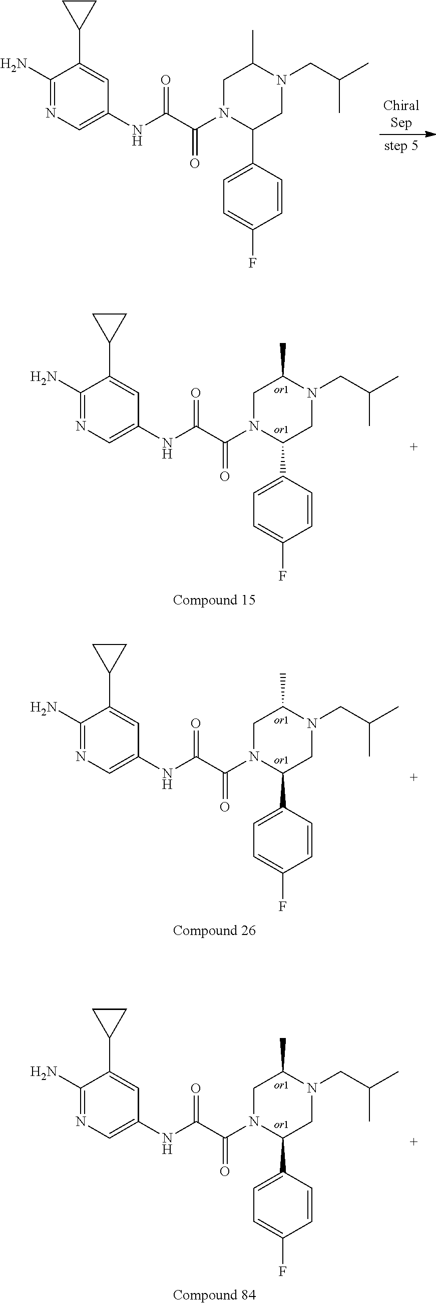 [SMILES: CC(C)CN1CC(c2ccc(F)cc2)N(C(=O)C(=O)Nc2cnc(N)c(C3CC3)c2)CC1C ; "ChiralSep" ; CC(C)CN1C[C@H](c2ccc(F)cc2)N(C(=O)C(=O)Nc2cnc(N)c(C3CC3)c2)C[C@H]1C ; CC(C)CN1C[C@@H](c2ccc(F)cc2)N(C(=O)C(=O)Nc2cnc(N)c(C3CC3)c2)C[C@@H]1C ; CC(C)CN1C[C@@H](c2ccc(F)cc2)N(C(=O)C(=O)Nc2cnc(N)c(C3CC3)c2)C[C@H]1C]

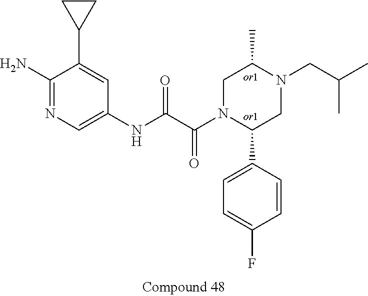 [SMILES: CC(C)CN1C[C@H](c2ccc(F)cc2)N(C(=O)C(=O)Nc2cnc(N)c(C3CC3)c2)C[C@@H]1C]

### Step 1: Synthesis of tert-butyl 2-(4-fluorophenyl)-4-isobutyl-5-methylpiperazine-1-carboxylate

Prepared by Scheme 1.1 step 8C. Yield: 300 mg (83.99%).

LCMS(ESI): [M-Boc]⁺ m/z: calcd 250.2; found 251.2; Rt=0.780 min.

### Step 2: Synthesis of 5-(4-fluorophenyl)-1-isobutyl-2-methylpiperazine

Prepared by Scheme 1.1 step 9. Yield: 200 mg of crude.

¹H NMR (500 MHz, CDCl₃) δ (ppm) 0.89 (d, 6H), 1.11 (m, 3H), 1.73 (m, 3H), 1.87 (m, 1H), 2.16 (m, 1H), 2.48 (m, 1H), 2.89 (m, 1H), 3.15 (m, 1H), 3.82 (m, 1H), 6.98 (d, 2H), 7.41 (d, 2H).

### Step 3: Synthesis of tert-butyl (3-cyclopropyl-5-(2-(2-(4-fluorophenyl)-4-isobutyl-5-methylpiperazin-1-yl)-2-oxoacetamido)pyridin-2-yl)carbamate

Prepared by Scheme 1.1 step 10A. Yield: 150 mg of crude.

LCMS(ESI): [M]⁺ m/z: calcd 553.2; found 554.2; Rt=1.072 min.

### Step 4: Synthesis of N-(6-amino-5-cyclopropylpyridin-3-yl)-2-(2-(4-fluorophenyl)-4-isobutyl-5-methylpiperazin-1-yl)-2-oxoacetamide

Prepared by Boc Group deprotection Method A. Yield: 50 mg (40.69%).

LCMS(ESI): [M]⁺ m/z: calcd 453.2; found 454.2; Rt=0.811 min.

### Step 4: Chiral Separation (Compound 26, Compound 84, Compound 48 and Compound 15)

Racemic N-(6-amino-5-cyclopropylpyridin-3-yl)-2-(2-(4-fluorophenyl)-4-isobutyl-5-methyl piperazin-1-yl)-2-oxoacetamide (50 mg, 110.24 umol) was chirally separated (Column: ChiralART YMC(250*20 mm, 5mkm); Mobile phase: Hexane-IPA-MeOH 80-10-10 Flow Rate:12 mL/min) to obtain N-(6-amino-5-cyclopropyl-3-pyridyl)-2-[(2R,5S)-2-(4-fluorophenyl)-4-isobutyl-5-methyl-piperazin-1-yl]-2-oxo-acetamide (8.65 mg, 19.07 umol, 17.30%) (RT=31.42 min) and N-(6-amino-5-cyclopropyl-3-pyridyl)-2-[(2R,5R)-2-(4-fluorophenyl)-4-isobutyl-5-methyl-piperazin-1-yl]-2-oxo-acetamide (6.93 mg, 15.28 umol, 13.86%) (RT=36.93 min). The rest of the mixture was chirally separated (Column: Chiralpak AS-H(250*20 mm, 5mkm); Mobile phase: Hexane-IPA-MeOH 90-5-5 Flow Rate:12 mL/min) to obtain N-(6-amino-5-cyclopropyl-3-pyridyl)-2-[(2S,5S)-2-(4-fluorophenyl)-4-isobutyl-5-methyl-piperazin-1-yl]-2-oxo-acetamide (6.39 mg, 14.09 umol, 12.78%) (RT=20.64 min) and N-(6-amino-5-cyclopropyl-3-pyridyl)-2-[(2S,5R)-2-(4-fluorophenyl)-4-isobutyl-5-methyl-piperazin-1-yl]-2-oxo-acetamide (9.26 mg, 20.42 umol, 18.52%) (RT=29.57 min).

Rel Time for Compound 26 in analytical conditions (column: IC, Hexane-IPA-MeOH, 60-20-20, 0.6 ml/min as mobile phase) 17.67 min and for Compound 84 20.36 min, for Compound 48 in analytical conditions (column: AS-H, Hexane-IPA-MeOH, 90-5-5, 0.6 ml/min as mobile phase) 19.76 min and for Compound 15 26.49 min.

Compound 26: Retention time: 17.67 min

¹H NMR (600 MHz, DMSO-d₆) δ (ppm) 0.45 (m, 2H), 0.79 (d, 3H), 0.83 (d, 3H), 0.88 (m, 2H), 0.94 (dd, 3H), 1.67 (m, 1H), 1.80 (m, 2H), 2.28 (m, 1H), 2.36 (m, 1H), 2.40 (m, 1H), 2.42 (m, 1H), 3.50 (d, 1H), 3.85 (dd, 1H), 5.32 (m, 1H), 5.87 (s, 2H), 7.17 (dt, 2H), 7.36 (m, 1H), 7.50 (m, 2H), 8.05 (dd, 1H), 10.51 (m, 1H).

LCMS(ESI): [M]⁺ m/z: calcd 453.2; found 454.2; Rt=0.957 min.

Compound 84: Retention time: 20.36 min 1H NMR (600 MHz, DMSO-d₆) δ (ppm) 0.45 (m, 2H), 0.81 (m, 6H), 0.88 (m, 2H), 0.93 (d, 3H), 1.70 (m, 2H), 2.11 (m, 2H), 2.93 (m, 4H), 3.75 (dd, 1H), 5.28 (m, 1H), 5.83 (s, 2H), 7.16 (dt, 2H), 7.35 (s, 1H), 7.52 (m, 2H), 8.04 (m, 1H), 10.50 (m, 1H).

LCMS(ESI): [M]⁺ m/z: calcd 453.2; found 454.2; Rt=1.837 min.

Compound 48: Retention time: 19.76 min 1H NMR (600 MHz, DMSO-d₆) δ (ppm) 0.45 (m, 2H), 0.81 (m, 6H), 0.88 (m, 2H), 0.93 (d, 3H), 1.70 (m, 2H), 2.11 (m, 2H), 2.93 (m, 4H), 3.75 (dd, 1H), 5.28 (m, 1H), 5.83 (s, 2H), 7.16 (dt, 2H), 7.35 (s, 1H), 7.52 (m, 2H), 8.04 (m, 1H), 10.50 (m, 1H).

LCMS(ESI): [M]⁺ m/z: calcd 453.2; found 454.2; Rt=2.234 min.

Compound 15: Retention time: 26.49 min

1H NMR (600 MHz, DMSO-d₆) δ (ppm) 0.45 (m, 2H), 0.79 (d, 3H), 0.83 (d, 3H), 0.88 (m, 2H), 0.94 (dd, 3H), 1.67 (m, 1H), 1.80 (m, 2H), 2.28 (m, 1H), 2.36 (m, 1H), 2.40 (m, 1H), 2.42 (m, 1H), 3.50 (d, 1H), 3.85 (dd, 1H), 5.32 (m, 1H), 5.87 (s, 2H), 7.17 (dt, 2H), 7.36 (m, 1H), 7.50 (m, 2H), 8.05 (dd, 1H), 10.51 (m, 1H).

LCMS(ESI): [M]⁺ m/z: calcd 453.2; found 454.2; Rt=2.319 min.

### Example 49. Compound 103 and Compound 125 2-(2-(4-fluorophenyl)-5-methyl-4-pivaloylpiperazin-1-yl)-2-oxo-N-(1H-pyrazolo[3,4-c]pyridin-4-yl)acetamide

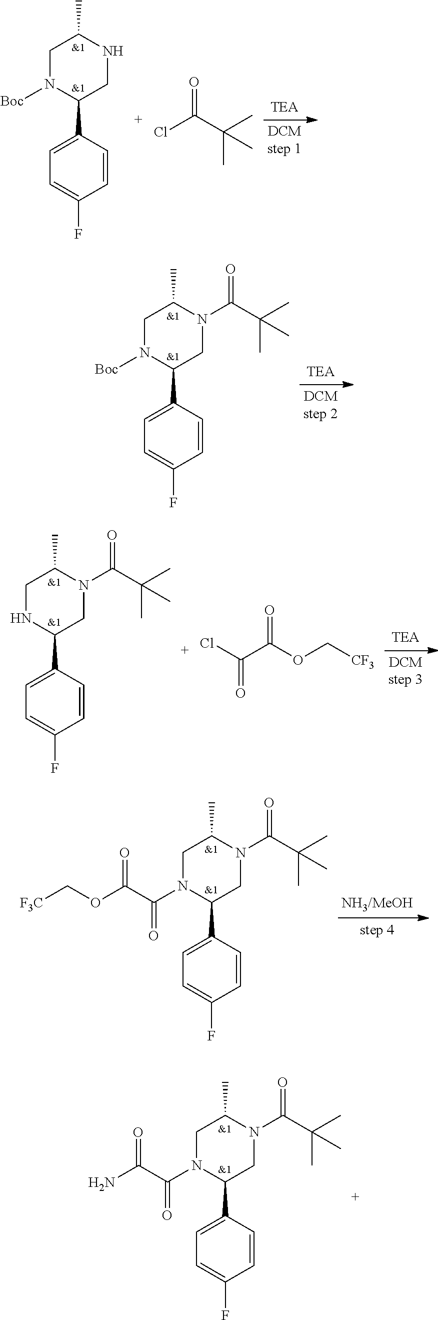

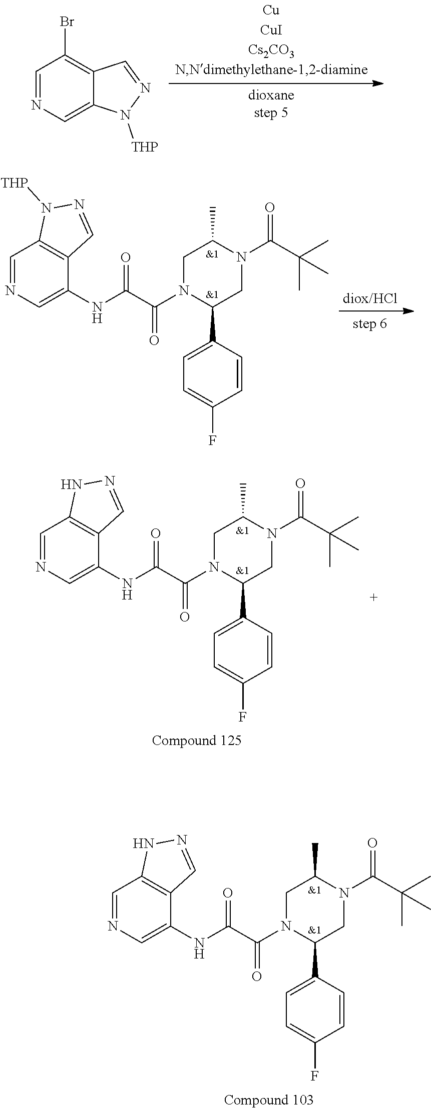

### Step 1: Synthesis of rac-(2R,5S)-tert-butyl 2-(4-fluorophenyl)-5-methyl-4-pivaloylpiperazine-1-carboxylate

Prepared by Scheme 1.1 step 8A. Yield: 2.63 g of crude.

LCMS(ESI): [M-t-Bu]⁺ m/z: calcd 322.2; found 323.2; Rt=1.614 min.

### Step 2: Synthesis of rac-1-((2R,5S)-5-(4-fluorophenyl)-2-methylpiperazin-1-yl)-2,2-dimethylpropan-1-one

Prepared by Scheme 1.1 step 9. Yield: 1.8 g of crude.

LCMS(ESI): [M]⁺ m/z: calcd 278.2; found 279.2; Rt=0.930 min.

### Step 3: Synthesis of rac-2,2,2-trifluoroethyl 2-((2R,5S)-2-(4-fluorophenyl)-5-methyl-4-pivaloylpiperazin-1-yl)-2-oxoacetate

Prepared by Scheme 1.3 step 1. Yield: 2.86 g of crude.

LCMS(ESI): [M]⁺ m/z: calcd 432.2; found 433.2; Rt=1.490 min.

### Step 4: Synthesis of rac-2-((2R,5S)-2-(4-fluorophenyl)-5-methyl-4-pivaloylpiperazin-1-yl)-2-oxoacetamide

Prepared by Scheme 1.3 step 2. Yield: 1.54 g of crude.

LCMS(ESI): [M]⁺ m/z: calcd 349.2; found 350.2; Rt=1.198 min.

### Step 5: Synthesis of rac-2-((2R,5S)-2-(4-fluorophenyl)-5-methyl-4-pivaloylpiperazin-1-yl)-2-oxo-N-(1-(tetrahydro-2H-pyran-2-yl)-1H-pyrazolo[3,4-c]pyridin-4-yl)acetamide

Prepared by Scheme 1.3 step 3A. Yield: 114.1 mg (48.27%).

HPLC conditions: Column: SunFire 100*19 mm, 5 microM; 2-10 min 40-50% MeOH+NH₃ 30 ml/min, loading pump 4 ml/min MeOH.

LCMS(ESI): [M]⁺ m/z: calcd 550.2; found 551.2; Rt=1.321 min.

### Step 6: Synthesis of 2-(2-(4-fluorophenyl)-5-methyl-4-pivaloylpiperazin-1-yl)-2-oxo-N-(1H-pyrazolo[3,4-c]pyridin-4-yl)acetamide (Compound 103 and Compound 125)

2-[(2S,5R)-4-(2,2-Dimethylpropanoyl)-2-(4-fluorophenyl)-5-methyl-piperazin-1-yl]-2-oxo-N-(1-tetrahydropyran-2-ylpyrazolo[3,4-c]pyridin-4-yl)acetamide (114.1 mg, 207.22 umol) was dissolved in dioxane (2 mL) and HCl/dioxane (5 mL) was added thereto. The resulting mixture was stirred overnight. The reaction mixture was concentrated in vacuum and purified by HPLC (2-10 min 30-45% MeOH+NH₃, 30 ml/min (loading pump 4 ml MeOH), target mass 466, column: SunFire 100*19 mm, 5 microM) to obtain 2-[(2S,5R)-4-(2,2-dimethylpropanoyl)-2-(4-fluorophenyl)-5-methyl-piperazin-1-yl]-2-oxo-N-(1H-pyrazolo[3, 4-c]pyridin-4-yl)acetamide (21.4 mg, 45.87 umol, 22.14% yield) and 2-[(2R,5R)-4-(2,2-dimethylpropanoyl)-2-(4-fluorophenyl)-5-methyl-piperazin-1-yl]-2-oxo-N-(¹H-pyrazolo[3, 4-c]pyridin-4-yl)acetamide (22.8 mg, 48.87 umol, 23.59% yield).

Compound 103: ¹H NMR (600 MHz, DMSO-d₆) δ (ppm) 1.07 (m, 3H), 1.23 (m, 9H), 3.15 (m, 1H), 3.58 (m, 1H), 4.43 (m, 3H), 5.15 (m, 1H), 7.15 (m, 2H), 7.41 (m, 2H), 8.43 (m, 2H), 8.79 (m, 1H), 11.15 (m, 1H), 13.70 (s, 1H).

LCMS(ESI): [M]⁺ m/z: calcd 466.2; found 467.2; Rt=3.099 min.

Compound 125: ¹H NMR (600 MHz, DMSO-d₆) δ (ppm) 1.08 (s, 9H), 1.27 (m, 3H), 2.28 (m, 1H), 2.84 (m, 1H), 3.82 (dd, 1H), 4.49 (m, 1H), 4.91 (m, 1H), 5.48 (m, 1H), 7.20 (m, 2H), 7.38 (m, 2H), 8.33 (m, 1H), 8.59 (m, 1H), 8.84 (m, 1H), 11.41 (s, 1H), 13.73 (s, 1H).

LCMS(ESI): [M]⁺ m/z: calcd 466.2; found 467.2; Rt=3.082 min.

### Example 50. Compound 102 and Compound 5 5-(2-(2-(4-fluorophenyl)-5-methyl-4-(1-methylcyclopropanecarbonyl)piperazin-1-yl)-2-oxoacetamido)-2-methoxynicotinamide

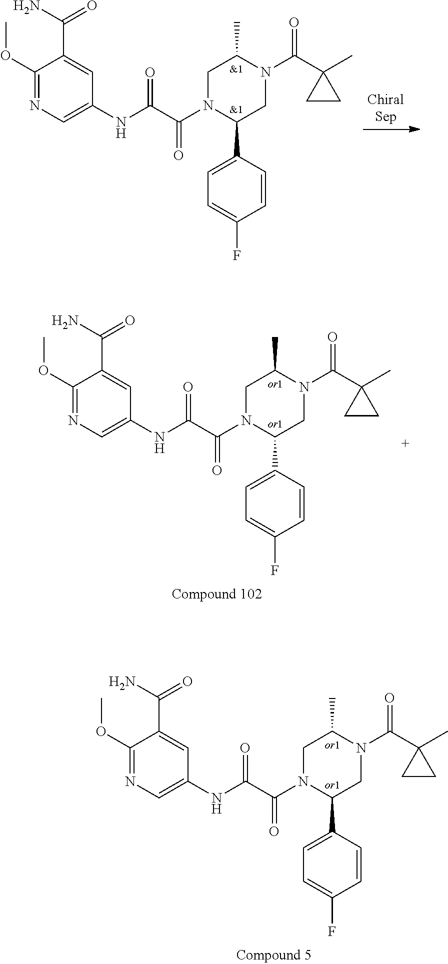

Racemic 5-(2-(2-(4-fluorophenyl)-5-methyl-4-(1-methylcyclopropanecarbonyl)piperazin-1-yl)-2-oxoacetamido)-2-methoxynicotinamide (23.8 mg, 47.84 umol) was chiral separated (Column: Chiralcel OD-H 250*20, 5—I Hexane-IPA-MeOH,80-10-10, 12 ml/min) to obtain 5-(2-((2S,5R)-2-(4-fluorophenyl)-5-methyl-4-(1-methylcyclopropanecarbonyl)piperazin-1-yl)-2-oxoacetamido)-2-methoxynicotinamide (12.04 mg, 50.58% yield) (RT=31.79 min) and 5-(2-((2R,5S)-2-(4-fluorophenyl)-5-methyl-4-(1-methylcyclopropanecarbonyl)piperazin-1-yl)-2-oxoacetamido)-2-methoxynicotinamide (9.68 mg, 40.67% yield) (RT=39.11 min).

Rel Time for Compound 102 in analytical conditions (column: OD-H, Hexane-IPA-MeOH, 60-20-20, 0.6 ml/min as mobile phase) 11.27 min and for Compound 5 13.02 min.

Compound 102: Retention time: 11.27 min

1H NMR (600 MHz, DMSO-d₆) δ (ppm) 0.36-0.56 (m, 2H), 0.59-0.72 (m, 1H), 0.73-1.02 (m, 1H), 1.05 (s, 3H), 1.06-1.38 (m, 4H), 2.86-3.21 (m, 1H), 3.34-3.79 (m, 1H), 3.93-3.99 (m, 3H), 4.00-4.51 (m, 1H), 4.50-4.98 (m, 1H), 5.29-5.90 (m, 1H), 7.14-7.23 (m, 2H), 7.26-7.41 (m, 2H), 7.69-7.79 (m, 2H), 8.42-8.62 (m, 2H), 10.89-11.29 (m, 1H).

LCMS(ESI): [M]⁺ m/z: calcd 497.2; found 498.2; Rt=2.745 min.

Compound 5: Retention time: 13.02 min

¹H NMR (600 MHz, DMSO-d₆) δ (ppm) 0.36-0.56 (m, 2H), 0.59-0.72 (m, 1H), 0.73-1.02 (m, 1H), 1.05 (s, 3H), 1.06-1.38 (m, 4H), 2.86-3.21 (m, 1H), 3.34-3.79 (m, 1H), 3.93-3.99 (m, 3H), 4.00-4.51 (m, 1H), 4.50-4.98 (m, 1H), 5.29-5.90 (m, 1H), 7.14-7.23 (m, 2H), 7.26-7.41 (m, 2H), 7.69-7.79 (m, 2H), 8.42-8.62 (m, 2H), 10.89-11.29 (m, 1H).

LCMS(ESI): [M]⁺ m/z: calcd 497.2; found 498.2; Rt=2.746 min.

### Example 5.1. Compound 86 N-(6-amino-5-cyclopropylpyridin-3-yl)-2-((2S,5R)-2-(4-fluorophenyl)-4-isobutyryl-5-methylpiperazin-1-yl)-2-oxoacetamide

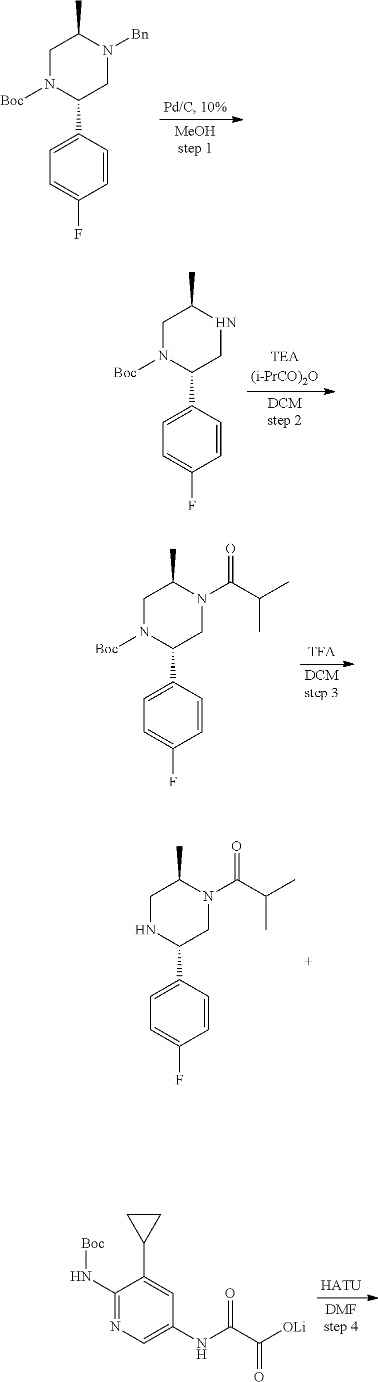

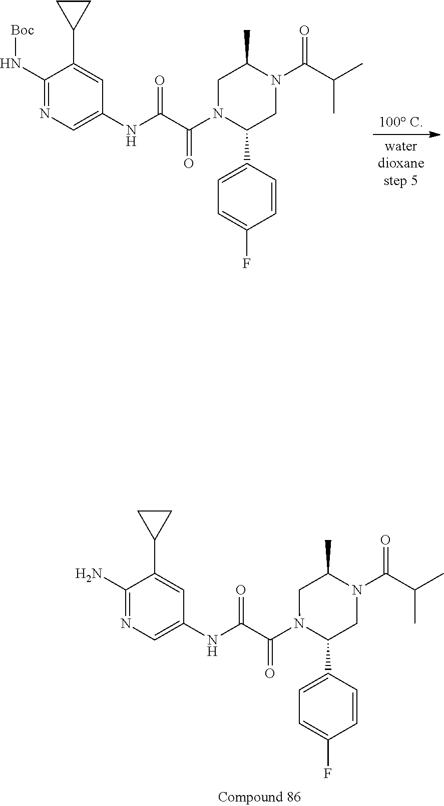 [SMILES: CC(C)C(=O)N1C[C@H](c2ccc(F)cc2)N(C(=O)C(=O)Nc2cnc(NC(=O)OC(C)(C)C)c(C3CC3)c2)C[C@H]1C ; "100° C." ; CC(C)C(=O)N1C[C@H](c2ccc(F)cc2)N(C(=O)C(=O)Nc2cnc(N)c(C3CC3)c2)C[C@H]1C]

The synthesis of (2S,5R)-1-benzyl-2-(4-fluorophenyl)-5-methylpiperazine is given by the following procedure. Borane dimethyl sulfide complex (2.66 g, 35.06 mmol, 3.33 mL) was added dropwise to the solution of (3S,6R)-4-benzoyl-3-(4-fluorophenyl)-6-methyl-piperazin-2-one (2.19 g, 7.01 mmol) in THF (30 mL). Resulting mixture was stirred at 65° C. for 18 hr. Then, it was cooled to rt and excess of borane was destroyed by dropwise addition of MeOH (10 ml). After H₂ evolution ceased, volatiles were removed under reduced pressure and residue was taken up in 2M aq. HCl (40 ml) and stirred at 50° C. for 40 minutes. Resulting cloudy solution was filtered and extracted with DCM (2×10 ml). DCM layers were discarded and aqueous layer was basified to pH≈11 with solid potassium hydroxide. Precipitated amine was extracted with DCM (2×25 ml). Organic layers were separated, dried over K₂CO₃ and concentrated in vacuum, affording (2S,5R)-1-benzyl-2-(4-fluorophenyl)-5-methyl-piperazine (1.31 g, 4.61 mmol, 65.70% yield).

LCMS(ESI): [M]⁺ m/z: calcd 284.2; found 285.2; Rt=1.049 min.

### Step 1: Synthesis of (2S,5R)-tert-butyl 2-(4-fluorophenyl)-5-methylpiperazine-1-carboxylate

tert-Butyl (2S,5R)-4-benzyl-2-(4-fluorophenyl)-5-methyl-piperazine-1-carboxylate (900.00 mg, 2.34 mmol) was dissolved in MeOH (5 mL) and palladium, 10% on carbon, Type 487, dry (747.32 ug, 7.02 umol) was added thereto. The reaction mixture was evacuated and backfilled three times with hydrogen and was hydrogenated at 1 atm (balloon) overnight. The catalyst was filtered off and the filtrate was concentrated in vacuum to obtain tert-butyl (2S,5R)-2-(4-fluorophenyl)-5-methyl-piperazine-1-carboxylate (428 mg, 1.45 mmol, 62.12% yield).

LCMS(ESI): [M-t-Bu]⁺ m/z: calcd 238.2; found 239.2; Rt=0.907 min.

### Step 2: Synthesis of (2S,5R)-tert-butyl 2-(4-fluorophenyl)-4-isobutyryl-5-methylpiperazine-1-carboxylate

tert-Butyl (2S,5R)-2-(4-fluorophenyl)-5-methyl-piperazine-1-carboxylate (428.00 mg, 1.45 mmol) and TEA (441.39 mg, 4.36 mmol, 607.97 uL) were mixed together in DCM (10 mL) and the resulting mixture was cooled to −5° C. in an ice/methanol bath. 2-methylpropanoyl 2-methylpropanoate (230.01 mg, 1.45 mmol, 241.10 uL) was added dropwise to the previous solution and the resulting mixture was allowed to warm to rt and stirred overnight. The reaction mixture was diluted with DCM (10 ml) and the resulting mixture was washed with water (2*5 ml), dried over Na₂SO₄, filtered and evaporated to obtain tert-butyl (2S,5R)-2-(4-fluorophenyl)-5-methyl-4-(2-methylpropanoyl)piperazine-1-carboxylate (477 mg, 1.31 mmol, 90.02% yield).

LCMS(ESI): [M-t-Bu]⁺ m/z: calcd 308.2; found 309.2; Rt=1.328 min.

### Step 3: Synthesis of 1-((2R,5S)-5-(4-fluorophenyl)-2-methylpiperazin-1-yl)-2-methylpropan-1-one

tert-Butyl (2S,5R)-2-(4-fluorophenyl)-5-methyl-4-(2-methylpropanoyl)piperazine-1-carboxylate (477.00 mg, 1.31 mmol) was dissolved in DCM (4 mL). TFA (746.15 mg, 6.54 mmol, 504.16 uL) was added dropwise to the resulting solution and stirred for 2 hr. The mixture was then basified with potassium carbonate (5 g in 10 ml H₂O), washed with DCM (2×5). The organic layer was combined, washed with water, dried over Na₂SO₄ and evaporated to give 1-[(2R,5S)-5-(4-fluorophenyl)-2-methyl-piperazin-1-yl]-2-methyl-propan-1-one (305 mg, 1.15 mmol, 88.16% yield).

LCMS(ESI): [M]⁺ m/z: calcd 264.2; found 265.2; Rt=0.837 min.

### Step 4: Synthesis of tert-butyl (3-cyclopropyl-5-(2-((2S,5R)-2-(4-fluorophenyl)-4-isobutyryl-5-methylpiperazin-1-yl)-2-oxoacetamido)pyridin-2-yl)carbamate

1-[(2R,5S)-5-(4-Fluorophenyl)-2-methyl-piperazin-1-yl]-2-methyl-propan-1-one (266.00 mg, 1.01 mmol) and 1-[(2R,5S)-5-(4-fluorophenyl)-2-methyl-piperazin-1-yl]-2-methyl-propan-1-one (266.00 mg, 1.01 mmol) were mixed in dry DMF (10 mL) at rt and the resulting mixture was stirred for 5 min. HATU (459.15 mg, 1.21 mmol) was added thereto and the resulting mixture was stirred at rt for 15 min. The resulting mixture was poured into water, extracted 3 times with EtOAc, combined organics were washed with water, brine and evaporated to obtained tert-butyl N-[3-cyclopropyl-5-[[2-[(2S,5R)-2-(4-fluorophenyl)-5-methyl-4-(2-methylpropanoyl)piperazin-1-yl]-2-oxo-acetyl]amino]-2-pyridyl]carbamate (534 mg, 940.72 umol, 93.48% yield). Crude product is used in the next step without purification. LCMS(ESI): [M]⁺ m/z: calcd 567.2; found 568.2; Rt=1.299 min.

### Step 5: Synthesis of N-(6-amino-5-cyclopropylpyridin-3-yl)-2-((2S,5R)-2-(4-fluorophenyl)-4-isobutyryl-5-methylpiperazin-1-yl)-2-oxoacetamide (Compound 86)

Prepared by Boc Group Deprotection Method A. Yield: 146.1 mg (33.22%).

HPLC conditions: Column: SunFire C18 100*19 mm, 5 microM; 2-10 min 30-45% MeCN 30 ml/min; (loading pump 4 ml/min MeCN).

Compound 86: ¹H NMR (600 MHz, DMSO-d₆) δ (ppm) 0.40-0.50 (m, 2H), 0.72-0.80 (m, 1H), 0.81-0.94 (m, 5H), 0.96-1.03 (m, 2H), 1.06-1.13 (m, 1H), 1.14-1.26 (m, 2H), 1.50-1.74 (m, 1H), 2.66-2.77 (m, 1H), 3.09-3.23 (m, 1H), 3.33-3.65 (m, 1H), 3.69-4.06 (m, 1H), 4.11-4.30 (m, 1H), 4.42-4.96 (m, 1H), 5.31-5.72 (m, 1H), 5.71-5.84 (m, 2H), 7.07-7.21 (m, 2H), 7.22-7.30 (m, 1H), 7.33-7.41 (m, 2H), 7.83-8.22 (m, 1H), 10.26-10.65 (m, 1H).

LCMS(ESI): [M]⁺ m/z: calcd 467.2; found 468.2; Rt=2.472 min.

### Example 52. Compound 81 N-(6-amino-5-ethylpyridin-3-yl)-2-((2S,5R)-5-methyl-2-phenyl-4-(1-(trifluoromethyl)cyclopropanecarbonyl)piperazin-1-yl)-2-oxoacetamide

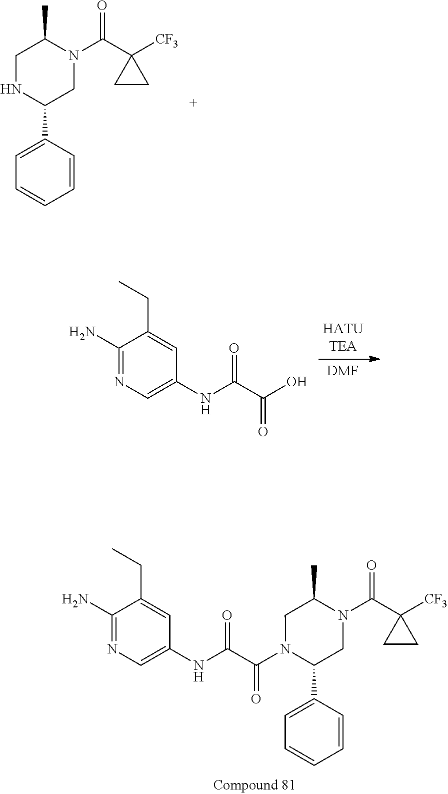

((2R,5 S)-2-methyl-5-phenylpiperazin-1-yl)(1-(trifluoromethyl)cyclopropyl)methanone is prepared by Scheme 1.1, step 9. Yield: 0.3 g (99.04%).

LCMS(ESI): [M]⁺ m/z: calcd 312.2; found 313.2; Rt=0.922 min.

Prepared by Scheme 1.1, step 10A. Yield: 25.6 mg (5.48%).

HPLC conditions: Column: Chromatorex C18 100*19 mm, 5 microM; 0-5 min 25-70% water-MeOH+0.1% FA 30 ml/min; (loading pump 4 ml/min MeCN).

Compound 81: ¹H NMR (600 MHz, DMSO-d₆) δ (ppm) 0.92-0.99 (m, 1H), 1.05-1.13 (m, 4H), 1.15-1.39 (m, 6H), 2.37-2.43 (m, 2H), 2.96-3.11 (m, 1H), 3.67-4.97 (m, 3H), 5.38-5.80 (m, 3H), 7.23-7.30 (m, 2H), 7.30-7.39 (m, 3H), 7.45-7.57 (m, 1H), 8.01-8.12 (m, 1H), 10.40-10.73 (m, 1H).

LCMS(ESI): [M]⁺ m/z: calcd 503.2; found 504.2; Rt=2.663 min.

### Example 53. Compound 104 N-(6-amino-5-methylpyridin-3-yl)-2-((2S,5R)-2-(4-fluorophenyl)-5-methyl-4-(1-methylcyclopropanecarbonyl)piperazin-1-yl)-2-oxoacetamide

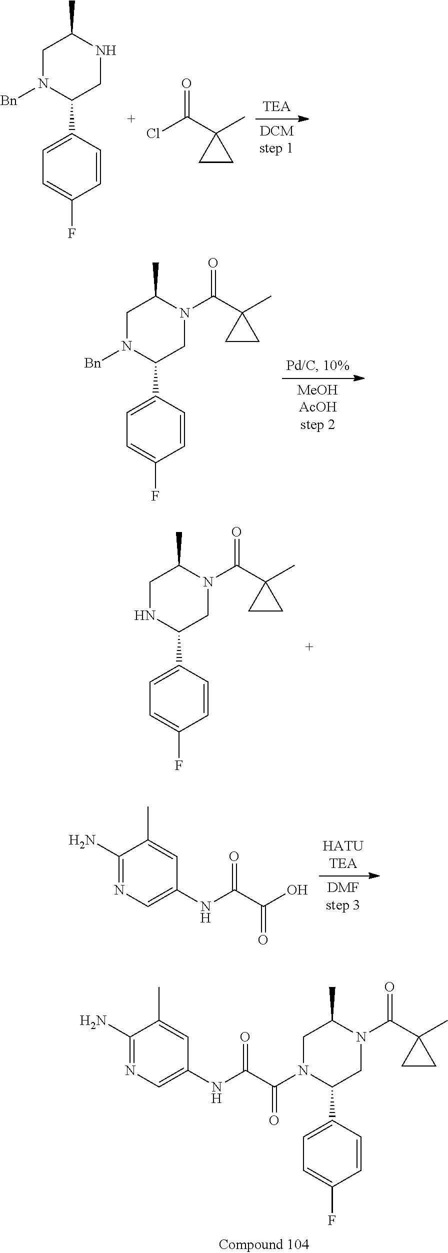

The synthesis of (2S,5R)-1-benzyl-2-(4-fluorophenyl)-5-methylpiperazine is given by the following procedure. Borane dimethyl sulfide complex (2.66 g, 35.06 mmol, 3.33 mL) was added dropwise to the solution of (3S,6R)-4-benzoyl-3-(4-fluorophenyl)-6-methyl-piperazin-2-one (2.19 g, 7.01 mmol) in THF (30 mL). Resulting mixture was stirred at 65° C. for 18 hr. Then, it was cooled to rt and excess of borane was destroyed by dropwise addition of MeOH (10 ml). After H₂ evolution ceased, volatiles were removed under reduced pressure and residue was taken up in 2M aq. HCl (40 ml) and stirred at 50° C. for 40 minutes. Resulting cloudy solution was filtered and extracted with DCM (2×10 ml). DCM layers were discarded and aqueous layer was basified to pH≈11 with solid potassium hydroxide. Precipitated amine was extracted with DCM (2×25 ml). Organic layers were separated, dried over K₂CO₃ and concentrated in vacuum, affording (2S,5R)-1-benzyl-2-(4-fluorophenyl)-5-methyl-piperazine (1.31 g, 4.61 mmol, 65.70% yield).

LCMS(ESI): [M]⁺ m/z: calcd 284.2; found 285.2; Rt=1.049 min.

### Step 1: Synthesis of ((2R,5S)-4-benzyl-5-(4-fluorophenyl)-2-methylpiperazin-1-yl)(1-methylcyclopropyl)methanone

1-Methylcyclopropanecarbonyl chloride (500.31 mg, 4.22 mmol) was added dropwise to the solution of (2S,5R)-1-benzyl-2-(4-fluorophenyl)-5-methyl-piperazine (1 g, 3.52 mmol) and TEA (711.68 mg, 7.03 mmol, 980.27 uL) in DCM (30 mL). Resulting mixture was stirred at 20° C. for 2 hr. Then, 15% aq. K₂CO₃ solution (20 ml) was added and stirring was continued for 10 min. After that, organic layer was separated, dried over K₂CO₃ and concentrated under reduced pressure, affording [(2R,5S)-4-benzyl-5-(4-fluorophenyl)-2-methyl-piperazin-1-yl]-(1-methylcyclopropyl)methanone (1.35 g, crude).

LCMS(ESI): [M]⁺ m/z: calcd 366.2; found 367.2; Rt=1.372 min.

### Step 2: Synthesis of ((2R,5S)-5-(4-fluorophenyl)-2-methylpiperazin-1-yl)(1-methylcyclopropyl)methanone

Palladium, 10% on carbon (350 mg, 328.89 umol, 10% purity) was added to the solution of [(2R,5S)-4-benzyl-5-(4-fluorophenyl)-2-methyl-piperazin-1-yl]-(1-methylcyclopropyl)methanone (1.35 g, 3.68 mmol) in MeOH (30 mL) and acetic acid (10 mL). Reaction flask was evacuated and backfilled with hydrogen (111.39 mg, 55.26 mmol) from attached balloon. Resulting mixture was stirred at 50° C. for 16 hr. Then, catalyst was filtered off and filtrate was concentrated under reduced pressure. Residue was partitioned between 10% aq. K₂CO₃ solution (20 ml) and DCM (40 ml). Organic layer was separated, dried over K₂CO₃ and concentrated in vacuum, affording [(2R,5S)-5-(4-fluorophenyl)-2-methyl-piperazin-1-yl]-(1-methylcyclopropyl)methanone (0.96 g, 3.47 mmol, 94.30% yield).

LCMS(ESI): [M]⁺ m/z: calcd 276.2; found 277.2; Rt=0.702 min.

### Step 3: Synthesis of N-(6-amino-5-methylpyridin-3-yl)-2-((2S,5R)-2-(4-fluorophenyl)-5-methyl-4-(1-methylcyclopropanecarbonyl)piperazin-1-yl)-2-oxoacetamide (Compound 104)

Prepared by Scheme 1.1 step 1OA. Yield: 130 mg (41.69%).

HPLC conditions: Column: YMC Triart C18 100*20 mm, 5 microM; 0-5 min 20-70% MeOH/water+0.1% NH₄OH 40 ml/min; (loading pump 4 ml/min MeOH).

Compound 104: ¹H NMR (600 MHz, DMSO-d₆) δ (ppm) 0.46 (m, 2H), 0.71 (m, 2H), 1.05 (m, 3H), 1.26 (m, 3H), 2.02 (m, 4H), 2.99 (m, 1H), 3.66 (m, 1H), 4.27 (m, 1H), 4.70 (m, 1H), 5.64 (m, 3H), 7.19 (m, 2H), 7.29 (m, 1H), 7.37 (m, 1H), 7.48 (m, 1H), 8.02 (m, 1H), 10.62 (m, 1H).

LCMS(ESI): [M]⁺ m/z: calcd 453.2; found 454.2; Rt=2.546 min.

### Example 54. Compound 119 N-(6-amino-5-methylpyridin-3-yl)-2-((2S,5R)-2-(3,4-dichlorophenyl)-5-methyl-4-(1-(trifluoromethyl)cyclopropanecarbonyl)piperazin-1-yl)-2-oxoacetamide

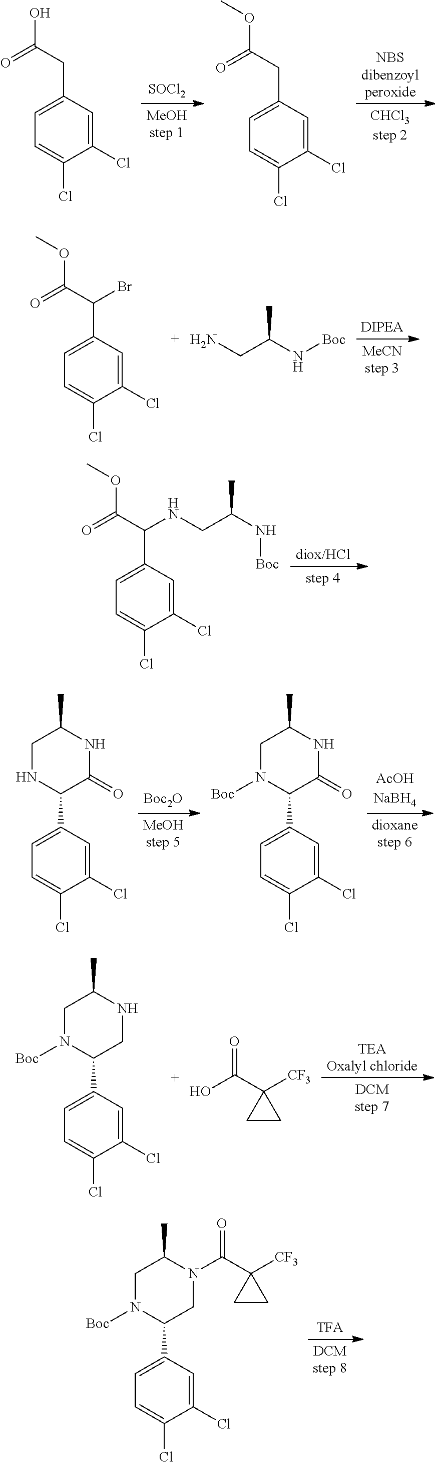

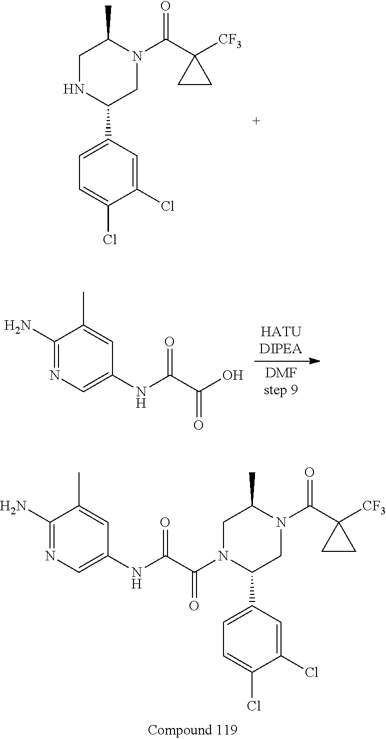 [SMILES: C[C@@H]1CN[C@@H](c2ccc(Cl)c(Cl)c2)CN1C(=O)C1(C(F)(F)F)CC1 ; Cc1cc(NC(=O)C(=O)O)cnc1N ; "HATUDIPEA" ; Cc1cc(NC(=O)C(=O)N2C[C@@H](C)N(C(=O)C3(C(F)(F)F)CC3)C[C@@H]2c2ccc(Cl)c(Cl)c2)cnc1N]

### Step 1: Synthesis of methyl 2-(3,4-dichlorophenyl)acetate

Thionyl chloride (8.70 g, 73.16 mmol) was added dropwise to the solution of 2-(3,4-dichlorophenyl)acetic acid (10 g, 48.77 mmol) in MeOH (50 mL) at 25° C. The reaction was mixed at 25° C. 16 hr. The solvent was evaporated under reduced pressure, the residue was diluted by DCM (30 ml.) and the organic phase was washed by aqueous NaHCO₃ (3*50 ml.), dried over Na₂SO₄ and evaporated under reduced pressure to give pure methyl 2-(3,4-dichlorophenyl)acetate (11.2 g, 51.13 mmol, 104.83% yield).

¹H NMR (500 MHz, DMSO-d₆) δ (ppm) 3.62 (s, 3H), 3.74 (s, 2H), 7.28 (d, 1H), 7.56 (m, 2H).

### Step 2: Synthesis of methyl 2-bromo-2-(3,4-dichlorophenyl)acetate

Methyl 2-(3,4-dichlorophenyl)acetate (11.2 g, 51.13 mmol), NBS (13.65 g, 76.69 mmol, 6.51 mL) and dibenzoyl peroxide (1.86 g, 7.67 mmol) were mixed in the chloroform (220 mL) and the reaction was refluxed at 65° C. 16 hr. After completion the solvent was evaporated under reduced pressure and the residue was diluted by DCM (80 ml.), the organic phase was washed by aqueous K₂CO₃ (3*50 ml.), water (3*50 ml.), organic phase was dried over Na₂SO₄ and evaporated under reduced pressure to give crude methyl 2-bromo-2-(3,4-dichlorophenyl)acetate (16.8 g, 56.38 mmol, 110.28% yield) that was used in the next step without purification.

¹H NMR (400 MHz, DMSO-d₆) δ (ppm) 3.75 (s, 3H), 5.98 (s, 1H), 7.55 (d, 1H), 7.66 (d, 1H), 7.81 (s, 1H).

### Step 3: Synthesis of methyl 2-(((R)-2-((tert-butoxycarbonyl)amino)propyl)amino)-2-(3,4-dichlorophenyl)acetate

Methyl 2-bromo-2-(3,4-dichlorophenyl)acetate (10 g, 33.56 mmol) and DIPEA (5.64 g, 43.63 mmol, 7.60 mL) were dissolved in MeCN (50 mL) and solution of tert-butyl N—[(1R)-2-amino-1-methyl-ethyl]carbamate (5.9 g, 33.86 mmol) in MeCN (50 mL) was added thereto. The reaction was mixed at 25° C. 16 hr. The reaction mixture was evaporated to dryness under reduced pressure and the residue was diluted by DCM (500 ml), washed by water (3*50 ml), dried over Na₂SO₄ and evaporated under reduced pressure to afford crude methyl 2-[[(2R)-2-(tert-butoxycarbonylamino)propyl]amino]-2-(3,4-dichlorophenyl)acetate (13 g, 33.22 mmol, 98.99% yield) that was used directly in the next step without additional purification.

LCMS(ESI): [M]⁺ m/z: calcd 391.2; found 392.2; Rt=1.332 min.

### Step 4: Synthesis of (3S,6R)-3-(3,4-dichlorophenyl)-6-methylpiperazin-2-one

Methyl 2-[[(2R)-2-(tert-butoxycarbonylamino)propyl]amino]-2-(3,4-dichlorophenyl)acetate (13 g, 33.22 mmol) was dissolved in dioxane/HCl (100 mL) and the reaction was mixed at 25° C. 16 hr. After completion the solvent was evaporated under reduced pressure and the residue was diluted by saturated aqueous K₂CO₃, aqueous phase was extracted by DCM (3*50 ml.), dried over Na₂SO₄ and evaporated under reduced pressure at 40° C. Then the residue was routed on rotary evaporator at 60° C. 1 hr until the red oil turned in yellow powder (mixture of cis- and trans- isomers). Recrystallization of the residue (6.3 g) from toluene (10 ml.) gives pure trans-isomer (3S, 6R)-3-(3,4-dichlorophenyl)-6-methyl-piperazin-2-one (1.7 g, 6.56 mmol, 19.75% yield).

LCMS(ESI): [M]⁺ m/z: calcd 259.2; found 260.2; Rt=0.559 min.

### Step 5: Synthesis of (2S,5R)-tert-butyl 2-(3,4-dichlorophenyl)-5-methyl-3-oxopiperazine-1-carboxylate

(3S,6R)-3-(3,4-Dichlorophenyl)-6-methyl-piperazin-2-one (1.7 g, 6.56 mmol) was dissolved in MeOH (20 mL) and tert-butoxycarbonyl tert-butyl carbonate (1.43 g, 6.56 mmol, 1.51 mL) was added thereto at 25° C. and mixed at 25° C. for 16 hr. Then the reaction mixture was evaporated under reduced pressure to dryness to obtain tert-butyl (2S,5R)-2-(3,4-dichlorophenyl)-5-methyl-3-oxo-piperazine-1-carboxylate (2.15 g, 5.98 mmol, 91.23% yield), structure was confirmed by 2D HNMR.

LCMS(ESI): [M]⁺ m/z: calcd 359.2; found 360.2; Rt=1.439 min.

### Step 6: Synthesis of (2S,5R)-tert-butyl 2-(3,4-dichlorophenyl)-5-methylpiperazine-1-carboxylate

Sodium borohydride (2.26 g, 59.85 mmol, 2.11 mL) was suspended in dioxane (10 mL) and this mixture was heated to 100° C. The solution of tert-butyl (2S,5R)-2-(3,4-dichlorophenyl)-5-methyl-3-oxo-piperazine-1-carboxylate (2.15 g, 5.98 mmol) and acetic acid (3.59 g, 59.85 mmol, 3.43 mL) in dioxane (5 mL) was added to the suspension maintaining 100° C. The reaction was stirred at 100° C. 1 hr. After completion the solvent was evaporated under reduced pressure, the residue was diluted by water (50 ml) slowly and the aqueous phase was extracted by DCM (3*30 ml), the combined organic phase was dried over Na₂SO₄ and evaporated under reduced pressure to obtain crude tert-butyl (2S,5R)-2-(3,4-dichlorophenyl)-5-methyl-piperazine-1-carboxylate (1.9 g, 5.50 mmol, 91.95% yield).

LCMS(ESI): [M-Boc]⁺ m/z: calcd 245.2; found 246.2; Rt=0.951 min.

### Step 7: Synthesis of (2S,5R)-tert-butyl 2-(3,4-dichlorophenyl)-5-methyl-4-(1-(trifluoromethyl)cyclopropanecarbonyl)piperazine-1-carboxylate

Oxalyl chloride (132.34 mg, 1.04 mmol, 90.96 L) was added in the solution of 1-(trifluoromethyl)cyclopropanecarboxylic acid (147.28 mg, 955.79 mol) in DCM (15 mL) followed by addition of 1-2 drop of DMF. The resulting mixture was mixed 2 hr at rt. Then reaction mixture was washed by NaHCO₃saturated aqueous solution (3*10 ml) to neutral pH, dried over Na₂SO₄, filtered and added at 0° C. to the stirred solution of tert-butyl (2S,5R)-2-(3,4-dichlorophenyl)-5-methyl-piperazine-1-carboxylate (0.3 g, 868.90 mol) and TEA (131.89 mg, 1.30 mmol, 181.66 L) in DCM (10 ml.). Reaction was warmed to 25° C. and stirred 16 hr at 25° C. Reaction mixture was washed by water (3*20 ml.), dried over Na₂SO₄ and evaporated to dryness under reduced pressure to give crude product tert-butyl (2S,5R)-2-(3,4-dichlorophenyl)-5-methyl-4-[1-(trifluoromethyl)cyclopropanecarbonyl]piperazine-1-carboxylate (0.3 g, 623.27 mol, 71.73% yield) that was directly used in the next step.

LCMS(ESI): [M-Boc]⁺ m/z: calcd 381.2; found 382.2; Rt=1.440 min.

### Step 8: Synthesis of ((2R,5S)-5-(3,4-dichlorophenyl)-2-methylpiperazin-1-yl)(1-(trifluoromethyl)cyclopropyl)methanone

tert-Butyl (2S,5R)-2-(3,4-dichlorophenyl)-5-methyl-4-[1-(trifluoromethyl)cyclopropanecarbonyl]piperazine-1-carboxylate (0.3 g, 623.27 mol) was dissolved in DCM (10 mL and TFA (355.33 mg, 3.12 mmol, 240.09 L) was added to reaction mixture at 0° C. then solution was allowed to warm to 25° C. and stirred 16 hr. Reaction mixture was diluted with DCM (20 ml) and washed by saturated aqueous NaHCO₃three times. The resulting organic phase was dried over Na₂SO₄ and evaporated under reduced pressure to afford crude [(2R,5S)-5-(3,4-dichlorophenyl)-2-methyl-piperazin-1-yl]-[1-(trifluoromethyl)cyclopropyl]methanone (0.17 g, 445.94 mol, 71.55% yield) that was used in the next step.

LCMS(ESI): [M]⁺ m/z: calcd 381.2; found 382.2; Rt=1.133 min.

### Step 9: Synthesis of N-(6-amino-5-methylpyridin-3-yl)-2-((2S,5R)-2-(3,4-dichlorophenyl)-5-methyl-4-(1-(trifluoromethyl)cyclopropanecarbonyl)piperazin-1-yl)-2-oxoacetamide (Compound 119)

Prepared by Scheme 1.1, step 10B. Yield: 54.5 mg (21.89%).

HPLC conditions: Column: XBridge BEH C18 100*19 mm, 5 microM; 0-5 min 30-70% MeOH/water+0.1% NH₄OH 30 ml/min; (loading pump 4 ml/min MeCN).

Compound 119: ¹H NMR (400 MHz, DMSO-d₆) δ (ppm) 1.03-1.30 (m, 7H), 1.99-2.07 (m, 4H), 2.87-3.25 (m, 1H), 3.76-4.13 (m, 1H), 4.29-5.06 (m, 2H), 5.41-5.82 (m, 3H), 7.17-7.37 (m, 1H), 7.44-7.58 (m, 2H), 7.63-7.69 (m, 1H), 8.01-8.08 (m, 1H), 10.62 (s, 1H).

LCMS(ESI): [M]⁺ m/z: calcd 558.2; found 559.2; Rt=2.463 min.

### Example 55. Compound 68 2-((2S,5R)-4-(3-(dimethylamino)-2,2-dimethylpropanoyl)-5-methyl-2-phenylpiperazin-1-yl)-2-oxo-N-(1H-pyrazolo[4,3-c]pyridin-7-yl)acetamide

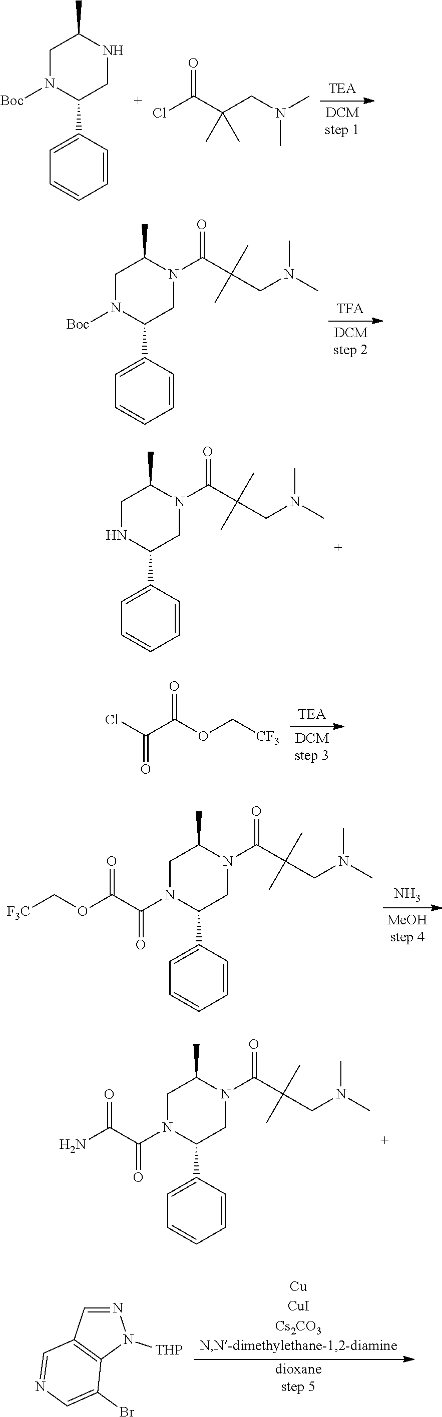

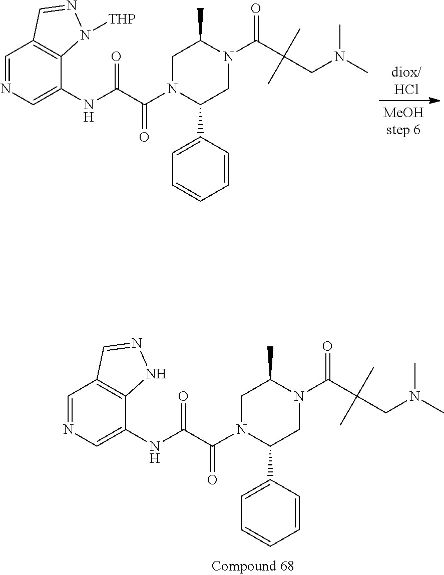

The synthesis of (2S,5R)-tert-butyl 5-methyl-2-phenylpiperazine-1-carboxylate is given by the following procedure. (2R)-1-Benzyl-2-methyl-5-phenyl-piperazine (2.1 g, 7.88 mmol) was dissolved in MeOH (50 mL), tert-butoxycarbonyl tert-butyl carbonate (2.06 g, 9.46 mmol, 2.17 mL) was added dropwise to the resulting mixture. The reaction mixture was stirred overnight. After completion, the reaction mixture was concentrated in vacuum. The residue was diluted with water (20 ml) and the resulting mixture was extracted with CHCl₃ (2*20 ml). Combined organic layers were washed with brine (25 ml), dried over Na₂SO₄, filtered and evaporated. The residue was purified by reverse phase HPLC chromatography (Column: Chromatorex 18 SMB 100-5T 100*19 mm, 5 microM; 0-6 min 20-50% water-MeCN 30 ml/min (loading pump 4 ml MeCN)) to give tert-butyl (2S,5R)-4-benzyl-5-methyl-2-phenyl-piperazine-1-carboxylate (0.75 g, 2.05 mmol, 25.96% yield).

LCMS(ESI): [M]⁺ m/z: calcd 366.2; found 367.2; Rt=3.202 min

### Step 1: Synthesis of (2S,5R)-tert-butyl 4-(3-(dimethylamino)-2,2-dimethylpropanoyl)-5-methyl-2-phenylpiperazine-1-carboxylate

To a stirred mixture of tert-butyl (2S,5R)-5-methyl-2-phenyl-piperazine-1-carboxylate (0.2 g, 723.66 mol) and TEA (366.14 mg, 3.62 mmol, 504.32 L) in DCM (10 mL) was cooled to 0° C. and 3-(dimethylamino)-2,2-dimethyl-propanoyl chloride (173.77 mg, 868.39 mol, HCl) was added in one portion, the resulting mixture was left to stir overnight at 14 hr. Upon completion of the reaction, mixture was washed with brine (2*20 ml), organic layer was dried over Na₂SO₄, filtered and concentrated under reduced pressure to obtain tert-butyl (2S,5R)-4-[3-(dimethylamino)-2,2-dimethyl-propanoyl]-5-methyl-2-phenyl-piperazine-1-carboxylate (0.3 g, crude).

LCMS(ESI): [M]⁺ m/z: calcd 403.2; found 404.2; Rt=1.016 min.

### Step 2: Synthesis of 3-(dimethylamino)-2,2-dimethyl-1-((2R,5S)-2-methyl-5-phenylpiperazin-1-yl)propan-1-one

tert-Butyl (2S,5R)-4-[3-(dimethylamino)-2,2-dimethyl-propanoyl]-5-methyl-2-phenyl-piperazine-1-carboxylate (0.3 g, 743.39 mol) was dissolved in DCM (9.81 mL) and then TFA (847.64 mg, 7.43 mmol, 572.73 L) was added dropwise, the resulting mixture was stirred at 25° C. for 16 hr. Upon completion of the reaction, the mixture was washed with water with K₂CO₃ (2 g in 20 ml water), organic layer was dried over Na₂SO₄, filtered and dried to obtain 3-(dimethylamino)-2,2-dimethyl-1-[(2R,5S)-2-methyl-5-phenyl-piperazin-1-yl]propan-1-one (0.2 g, crude).

LCMS(ESI): [M]⁺ m/z: calcd 303.2; found 304.2; Rt=0.386 min.

### Step 3: Synthesis of 2,2,2-trifluoroethyl 2-((2S,5R)-4-(3-(dimethylamino)-2,2-dimethylpropanoyl)-5-methyl-2-phenylpiperazin-1-yl)-2-oxoacetate

3-(Dimethylamino)-2,2-dimethyl-1-[(2R,5S)-2-methyl-5-phenyl-piperazin-1-yl]propan-1-one (0.2 g, 659.11 μmol) and TEA (100.04 mg, 988.66 μmol, 137.80 L) were mixed together in DCM (9.93 mL) and the resulting solution was cooled to 5° C. in an ice bath. 2,2,2-Trifluoroethyl 2-chloro-2-oxo-acetate (138.12 mg, 725.02 mol) was added dropwise to the previous solution and the resulting mixture was allowed to warm to rt and stirred overnight. Upon completion of the reaction, the mixture was washed with brine (2*20 ml), organic layer was dried over Na₂SO₄, filtered and concentrated in vacuum to affording 2,2,2-trifluoroethyl 2-[(2S,5R)-4-[3-(dimethylamino)-2,2-dimethyl-propanoyl]-5-methyl-2-phenyl-piperazin-1-yl]-2-oxo-acetate (0.3 g, crude).

LCMS(ESI): [M]⁺ m/z: calcd 457.2; found 458.2; Rt=0.954 min.

### Step 4: Synthesis of 2-((2S,5R)-4-(3-(dimethylamino)-2,2-dimethylpropanoyl)-5-methyl-2-phenylpiperazin-1-yl)-2-oxoacetamide

2,2,2-Trifluoroethyl 2-[(2S,5R)-4-[3-(dimethylamino)-2,2-dimethyl-propanoyl]-5-methyl-2-phenyl-piperazin-1-yl]-2-oxo-acetate (0.3 g, 655.76 mol) was dissolved in NH₃/MeOH (10 mL) and the resulting mixture was left to stir at 25° C. for 16 hr. Upon completion of the reaction, solvent was evaporated to dryness to obtain 2-[(2S,5R)-4-[3-(dimethylamino)-2,2-dimethyl-propanoyl]-5-methyl-2-phenyl-piperazin-1-yl]-2-oxo-acetamide (0.3 g, crude).

LCMS(ESI): [M]⁺ m/z: calcd 374.2; found 375.2; Rt=0.761 min.

### Step 5: Synthesis of 2-((2S,5R)-4-(3-(dimethylamino)-2,2-dimethylpropanoyl)-5-methyl-2-phenylpiperazin-1-yl)-2-oxo-N-(1-(tetrahydro-2H-pyran-2-yl)-1H-pyrazolo[4,3-c]pyridin-7-yl)acetamide

2-Oxo-2-[(2S,5R)-4-[3-(dimethylamino)-2,2-dimethyl-propanoyl]-5-methyl-2-phenyl-piperazin-1-yl]acetamide (0.2 g, 534.08 μmol), 7-bromo-1-tetrahydropyran-2-yl-pyrazolo[4,3-c]pyridine (150.68 mg, 534.08 μmol), Cu (33.94 mg, 534.08 mol), cesium carbonate (348.03 mg, 1.07 mmol), CuI (101.72 mg, 534.08 mol, 18.10 L) and (IS,2S)—N,N′-bis-methyl-1,2-cyclohexane-diamine (113.95 mg, 801.12 mol, 126.33 L) were mixed in dioxane (4 mL) under argon. The resulting mixture was allowed to stir at 100° C. for 18 hr in vial. Upon completion of the reaction, dioxane was evaporated and residue was subjected by HPLC (column: YMC Triart C18 100×20 mm, 5 um; mobile phase: 30-30-80% 0-5 min H₂O/MeOH/0.1% NH₄OH, flow rate: 30 ml/min (loading pump 4 ml/min MeOH), affording 2-oxo-2-[(2S,5R)-4-[3-(dimethylamino)-2,2-dimethyl-propanoyl]-5-methyl-2-phenyl-piperazin-1-yl]-N-(1-tetrahydropyran-2-ylpyrazolo[4,3-c]pyridin-7-yl)acetamide (46.20 mg, crude) on two portions.

LCMS(ESI): [M]⁺ m/z: calcd 575.2; found 576.2; Rt=1.565 min.

### Step 6: Synthesis of 2-((2S,5R)-4-(3-(dimethylamino)-2,2-dimethylpropanoyl)-5-methyl-2-phenylpiperazin-1-yl)-2-oxo-N-(1H-pyrazolo[4,3-c]pyridin-7-yl)acetamide (Compound 68)

2-Oxo-2-[(2S,5R)-4-[3-(dimethylamino)-2,2-dimethyl-propanoyl]-5-methyl-2-phenyl-piperazin-1-yl]-N-(1-tetrahydropyran-2-ylpyrazolo[4,3-c]pyridin-7-yl)acetamide (46.20 mg, 80.25 mol) was dissolved in a mixture of MeOH (1.5 mL) and diox/HCl (1.5 mL). The resulting clear solution was left to stir for 2 hr at 25° C. After that time, solvent was evaporated to dryness. Crude product was subjected by HPLC (column: Chromatorex 18 SMB100-5T 100×19 mm, 5 um; mobile phase: 0-0-25% 0-1-5 min H₂O/MeCN/0.1% FA, flow rate: 30 ml/min (loading pump 4 ml/min MeCN), affording 2-oxo-N-(1H-pyrazolo[4,3-c]pyridin-7-yl)-2-[(2S,5R)-4-[3-(dimethylamino)-2,2-dimethyl-propanoyl]-5-methyl-2-phenyl-piperazin-1-yl]acetamide (15.6 mg, 31.73 μmol, 39.54% yield, 2HCl).

Compound 68: ¹H NMR (600 MHz, DMSO-d₆) δ (ppm) 1.00-1.14 (m, 8H), 1.30 (s, 3H), 1.92 (s, 6H), 2.26 (dd, 1H), 2.67-2.78 (m, 1H), 3.95-4.21 (m, 1H), 4.34-4.51 (m, 1H), 5.01-5.71 (m, 1H), 7.18-7.33 (m, 2H), 7.34-7.39 (m, 3H), 8.07-8.39 (m, 2H), 8.40-8.53 (m, 1H), 8.80-8.99 (m, 1H), 11.22 (s, 1H), 13.11 (s, 1H).

LCMS(ESI): [M]⁺ m/z: calcd 491.2; found 492.2; Rt=0.927 min.

### Example 56. Compound 38 N-(6-amino-5-methylpyridin-3-yl)-2-((2S,5R)-2-(3,4-difluorophenyl)-5-methyl-4-(1-(trifluoromethyl)cyclopropanecarbonyl)piperazin-1-yl)-2-oxoacetamide

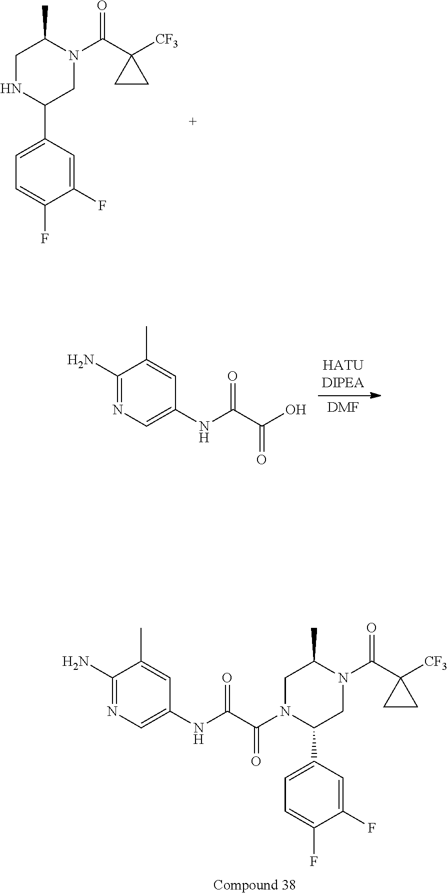

((2R)-5-(3,4-difluorophenyl)-2-methylpiperazin-1-yl)(1-(trifluoromethyl)cyclopropyl)methanone is prepared by general procedure scheme 1.1 step 9.

Yield: 0.13 g (83.68%).

LCMS(ESI): [M]⁺ m/z: calcd 348.2; found 349.2; Rt=0.989 min.

Prepared by general procedure scheme 1.1, step 10B. Yield: 14 mg (13.26%).

HPLC conditions: Column: YMC Triart C18 100*20 mm, 5 microM; 0-5 min 40-60% MeOH/water+0.1% NH₄OH 30 ml/min; (loading pump 4 ml/min MeOH).

Compound 38: ¹H NMR (600 MHz, DMSO-d₆) δ (ppm) 1.09 (m, 4H), 1.28 (m, 4H), 2.02 (m, 3H), 2.95 (m, 1H), 3.90 (m, 1H), 4.47 (m, 1H), 4.91 (m, 1H), 5.65 (m, 3H), 7.13 (m, 1H), 7.35 (m, 1H), 7.43 (t, 1H), 7.51 (m, 1H), 8.03 (m, 1H), 10.64 (m, 1H).

LCMS(ESI): [M]⁺ m/z: calcd 525.2; found 526.2; Rt=2.599 min.

### Example 57. Compound 30 N-(6-amino-5-methylpyridin-3-yl)-2-((2S,5R)-5-methyl-2-(m-tolyl)-4-(1-(trifluoromethyl)cyclopropanecarbonyl)piperazin-1-yl)-2-oxoacetamide

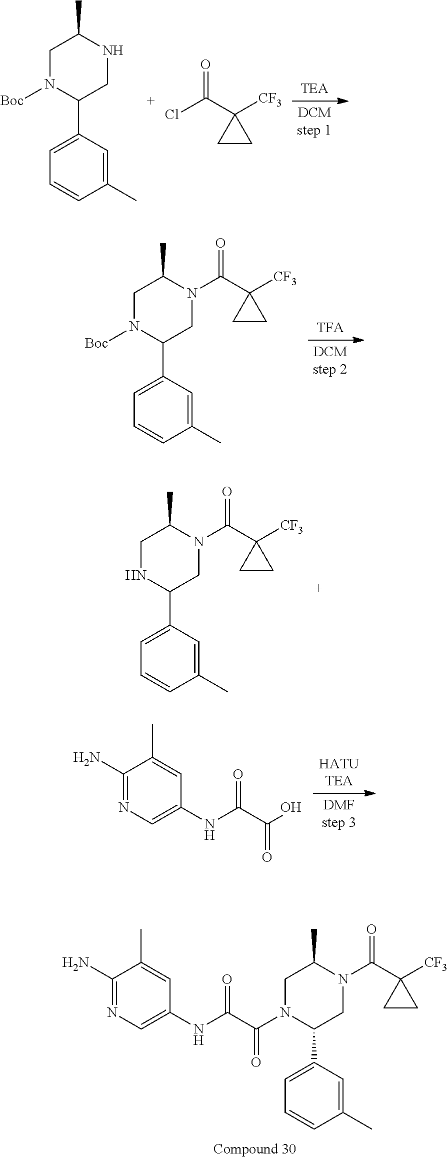

### Step 1: Synthesis of (5R)-tert-butyl 5-methyl-2-(m-tolyl)-4-(1-(trifluoromethyl)cyclopropanecarbonyl)piperazine-1-carboxylate

The synthesis of (5R)-tert-butyl 5-methyl-2-(m-tolyl)piperazine-1-carboxylate is prepared by general procedure scheme 1.1 step 7. Yield: 2.52 g of crude.

LCMS(ESI): [M]⁺ m/z: calcd 290.2; found 291.2; Rt=0.960 min.

Prepared by Scheme 1.1, step 8A. Yield: 2.46 g of crude.

LCMS(ESI): [M-t-Bu]⁺ m/z: calcd 370.2; found 371.2; Rt=1.470 min.

### Step 2: Synthesis of ((2R)-2-methyl-5-(m-tolyl)piperazin-1-yl)(1-(trifluoromethyl)cyclopropyl)methanone

Prepared by Scheme 1.1, step 9. Yield: 1.54 g of crude.

LCMS(ESI): [M]⁺ m/z: calcd 326.2; found 327.2; Rt=0.857 min.

### Step 3: Synthesis of N-(6-amino-5-methylpyridin-3-yl)-2-((2S,5R)-5-methyl-2-(m-tolyl)-4-(1-(trifluoromethyl)cyclopropanecarbonyl)piperazin-1-yl)-2-oxoacetamide (Compound 30)

Prepared by Scheme 1.1, step 10A. Yield: 108.1 mg (14.01%).

HPLC conditions: Column: SunFire C18 100*19 mm, 5 microM; 2-10 min 40-50% water/MeCN 30 ml/min; (loading pump 4 ml/min MeCN).

Compound 30: ¹H NMR (600 MHz, DMSO-d₆) δ (ppm) 0.92-1.00 (m, 1H), 1.13-1.33 (m, 6H), 2.11-2.19 (m, 3H), 2.21-2.29 (m, 3H), 2.64-3.09 (m, 1H), 3.65-3.98 (m, 1H), 4.00-4.18 (m, 1H), 4.27-4.50 (m, 1H), 4.49-5.02 (m, 1H), 5.12-5.89 (m, 1H), 6.98-7.13 (m, 3H), 7.18-7.26 (m, 1H), 7.47-7.77 (m, 2H), 7.81-7.94 (m, 1H), 8.24-8.37 (m, 1H), 10.52-11.28 (m, 1H).

LCMS(ESI): [M]⁺ m/z: calcd 503.2; found 504.2; Rt=2.333 min.

### Example 58. Compound 36 and Compound 73 N-(6-amino-5-ethylpyridin-3-yl)-2-(4-(2-cyclopropylacetyl)-2-(4-fluorophenyl)-5-methylpiperazin-1-yl)-2-oxoacetamide

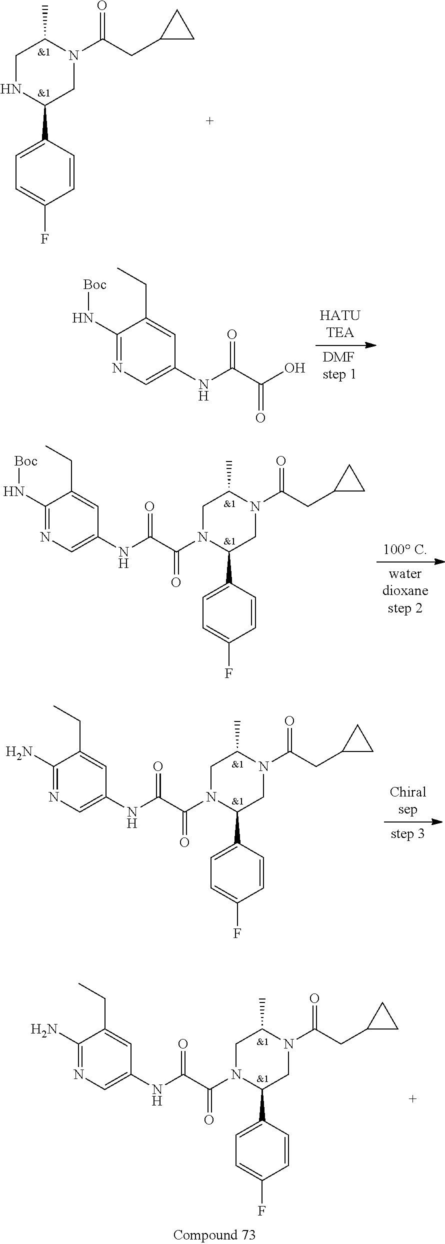 [SMILES: C[C@H]1CN[C@H](c2ccc(F)cc2)CN1C(=O)CC1CC1 ; CCc1cc(NC(=O)C(=O)O)cnc1NC(=O)OC(C)(C)C ; CCc1cc(NC(=O)C(=O)N2C[C@H](C)N(C(=O)CC3CC3)C[C@H]2c2ccc(F)cc2)cnc1NC(=O)OC(C)(C)C ; CCc1cc(NC(=O)C(=O)N2C[C@H](C)N(C(=O)CC3CC3)C[C@H]2c2ccc(F)cc2)cnc1N ; CCc1cc(NC(=O)C(=O)N2C[C@H](C)N(C(=O)CC3CC3)C[C@H]2c2ccc(F)cc2)cnc1N]

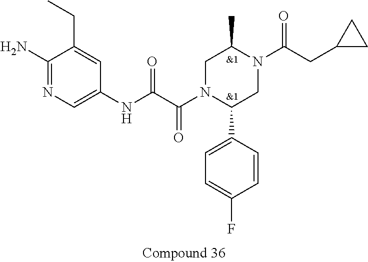 [SMILES: CCc1cc(NC(=O)C(=O)N2C[C@@H](C)N(C(=O)CC3CC3)C[C@@H]2c2ccc(F)cc2)cnc1N]

rac-2-cyclopropyl-1-((2R,5S)-5-(4-fluorophenyl)-2-methylpiperazin-1-yl)ethanone is prepared by Scheme 1.1, Step 9. Yield: 0.25 g of crude.

LCMS(ESI): [M]⁺ m/z: calcd 276.2; found 277.2; Rt=0.719 min.

### Step 1: Synthesis of rac-tert-butyl (5-(2-((2R,5S)-4-(2-cyclopropylacetyl)-2-(4-fluorophenyl)-5-methylpiperazin-1-yl)-2-oxoacetamido)-3-ethylpyridin-2-yl)carbamate

Prepared by Scheme 1.1, Step 10A. Yield: 74 mg (14.41%).

HPLC conditions: Column: SunFire 100*19 mm, 5 microM; 2-10 min 30-45% MeCN 30 ml/min (loading pump 4 ml MeCN).

LCMS(ESI): [M]⁺ m/z: calcd 567.2; found 568.2; Rt=1.473 min.

### Step 2: Synthesis of N-(6-amino-5-ethylpyridin-3-yl)-2-(4-(2-cyclopropylacetyl)-2-(4-fluorophenyl)-5-methylpiperazin-1-yl)-2-oxoacetamide

Prepared by Boc Group Deprotection Method A. Yield: 60 mg (98.44%).

LCMS(ESI): [M]⁺ m/z: calcd 467.2; found 468.2; Rt=2.598 min.

### Step 3: Chiral Separation (Compound 36 and Compound 73)

Racemic N-(6-amino-5-ethyl-3-pyridyl)-2-[(2S,5R)-4-(2-cyclopropylacetyl)-2-(4-fluorophenyl)-5-methyl-piperazin-1-yl]-2-oxo-acetamide (0.06 g, 128.33 umol) was chiral separated (Chiralcel OJ-H (250*20, 5 mkm), Hexane-IPA-MeOH,60-20-20, 12 ml/min) to obtain N-(6-amino-5-ethyl-3-pyridyl)-2-[(2S,5R)-4-(2-cyclopropylacetyl)-2-(4-fluorophenyl)-5-methyl-piperazin-1-yl]-2-oxo-acetamide (0.032 g, 68.44 umol, 53.33% yield) (RT=20.38 min) and N-(6-amino-5-ethyl-3-pyridyl)-2-[(2R,5S)-4-(2-cyclopropylacetyl)-2-(4-fluorophenyl)-5-methyl-piperazin-1-yl]-2-oxo-acetamide (0.032 g, 68.44 umol, 53.33% yield) (RT=38.23 min).

Rel Time for Compound 36 in analytical conditions (Chiralcel OJ-H (250*4.6, 5 mkm), Hexane-IPA-MeOH, 60-20-20, 0.6 ml/min as mobile phase) 14.55 min and for Compound 73 29.79 min.

Compound 36: Retention time: 14.55 min

1H NMR (DMSO-d₆, 600 MHz): δ (ppm) 0.01 (m, 2H), 0.38 (m, 2H), 0.88 (m, 1H), 1.14 (m, 6H), 1.98 (m, 1H), 2.39 (m, 4H), 2.91 (m, 1H), 3.64 (m, 1H), 4.03 (m, 1H), 4.93 (m, 1H), 5.56 (m, 3H), 7.18 (m, 2H), 7.31 (m, 1H), 7.39 (m, 1H), 7.50 (m, 1H), 8.06 (m, 1H), 10.53 (m, 1H)

LCMS(ESI): [M]⁺ m/z: calcd 467.2; found 468.2; Rt=1.113 min.

Compound 73: Retention time: 29.79 min

1H NMR (DMSO-d₆, 600 MHz): δ (ppm) 0.01 (m, 2H), 0.38 (m, 2H), 0.88 (m, 1H), 1.14 (m, 6H), 1.98 (m, 1H), 2.39 (m, 4H), 2.91 (m, 1H), 3.64 (m, 1H), 4.03 (m, 1H), 4.93 (m, 1H), 5.56 (m, 3H), 7.18 (m, 2H), 7.31 (m, 1H), 7.39 (m, 1H), 7.50 (m, 1H), 8.06 (m, 1H), 10.53 (m, 1H)

LCMS(ESI): [M]⁺ m/z: calcd 467.2; found 468.2; Rt=1.112 min.

### Example 59. Compound 32 N-(6-amino-5-ethylpyridin-3-yl)-2-((2S,5R)-4-isobutyl-5-methyl-2-phenylpiperazin-1-yl)-2-oxoacetamide (

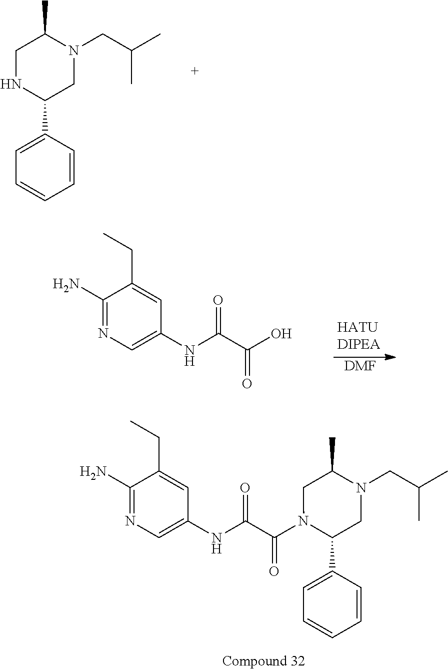

The synthesis of (2R,5S)-1-isobutyl-2-methyl-5-phenylpiperazine is given by the following procedure. LAH (64.71 mg, 1.70 mmol) was suspended in THF (5 mL) and the resulting suspension was heated to reflux. A solution of 2-methyl-1-[(2R,5S)-2-methyl-5-phenyl-piperazin-1-yl]propan-1-one (0.14 g, 568.30 umol) in THF (5 mL) was added dropwise to the previous suspension, maintaining a gentle reflux. After addition completed, the reaction mixture was refluxed for 16 hr and then allowed to cool to rt and stirred overnight. After completion, water (0.1 ml) was carefully added dropwise to the precooled reaction mixture followed by addition of a aq. KOH solution (0.1 ml) and water (0.2 ml). The resulting mixture was stirred for 30 min and filtered. A filter cake was rinsed with THF (5 ml) and the filtrate was concentrated in vacuum to afford (2R,5S)-1-isobutyl-2-methyl-5-phenyl-piperazine (0.1 g, 430.36 umol, 75.73% yield). LCMS(ESI): [M]⁺ m/z: calcd 232.2; found 233.2; Rt=0.817 min.

Prepared by Scheme 1.1, step 10B. Yield: 65.1 mg (23.81%).

HPLC conditions: Column: YMC Triart C18 100*20 mm, 5 microM; 0-1-6 min 50-50-100% water-MeOH+0.1% NH₄OH 30 ml/min; (loading pump 4 ml/min MeOH).

Compound 32: ¹H NMR (600 MHz, DMSO-d₆) δ (ppm) 0.75-0.84 (m, 6H), 0.92-0.98 (m, 3H), 1.06-1.15 (m, 3H), 1.73 (m, 1H), 2.08-2.16 (m, 2H), 2.37-2.41 (m, 2H), 2.82-3.38 (m, 4H), 3.47-4.03 (m, 1H), 5.08-5.49 (m, 1H), 5.60-5.66 (m, 2H), 7.21-7.28 (m, 1H), 7.30-7.38 (m, 2H), 7.42-7.53 (m, 3H), 8.02-8.07 (m, 1H), 10.51 (s, 1H).

LCMS(ESI): [M]⁺ m/z: calcd 423.2; found 424.2; Rt=1.912 min.

### Example 60. Compound 40 N-(6-amino-5-methylpyridin-3-yl)-2-((2S,5R)-5-methyl-4-(1-methylcyclopropanecarbonyl)-2-(m-tolyl)piperazin-1-yl)-2-oxoacetamide

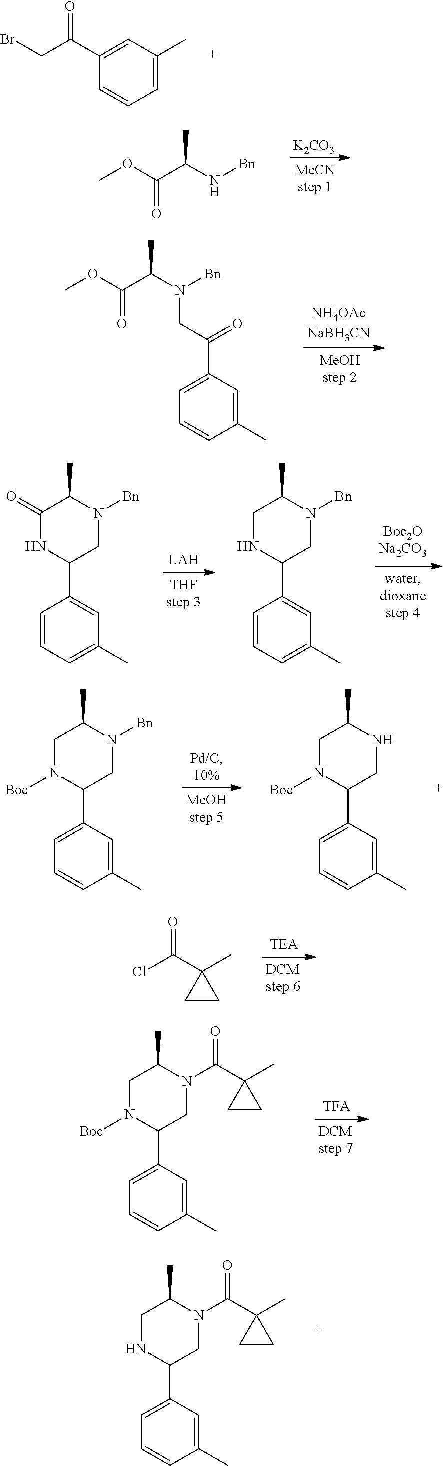

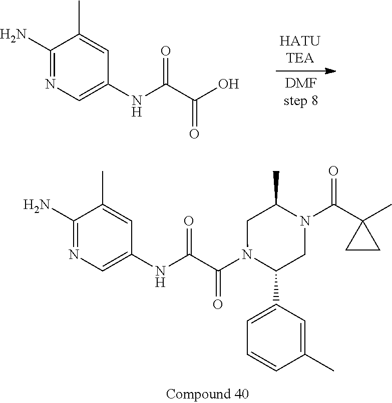 [SMILES: Cc1cc(NC(=O)C(=O)O)cnc1N ; Cc1cccc([C@H]2CN(C(=O)C3(C)CC3)[C@H](C)CN2C(=O)C(=O)Nc2cnc(N)c(C)c2)c1]

### Step 1: Synthesis of (R)-methyl 2-(benzyl(2-oxo-2-(m-tolyl)ethyl)amino)propanoate

Methyl (2R)-2-(benzylamino)propanoate (7.66 g, 39.66 mmol) was dissolved in MeCN (170 mL) and potassium carbonate—granular (6.58 g, 47.59 mmol, 2.87 mL) was added thereto. A solution of 2-bromo-1-(m-tolyl)ethanone (8.45 g, 39.66 mmol) in MeCN (30 mL) was added to the previous mixture and the resulting mixture was vigorously stirred overnight. The reaction mixture was concentrated in vacuum and water (150 ml) was added to the residue. The resulting mixture was extracted with MTBE (2*150 ml) an combined layers were dried over Na₂SO₄, filtered and concentrated in vacuum to obtain methyl (2R)-2-[benzyl-[2-(m-tolyl)-2-oxo-ethyl]amino]propanoate (13.65 g, crude).

LCMS(ESI): [M]⁺ m/z: calcd 325.2; found 326.2; Rt=1.422 min.

### Step 2: Synthesis of (3R)-4-benzyl-3-methyl-6-(m-tolyl)piperazin-2-one

Prepared by Scheme 1.1, step 4. Yield: 8.14 g (65.93%).

CC conditions: The crude product was purified by silica gel with EtOAc/Hexane (2:1) as an eluent mixture.

LCMS(ESI): [M]⁺ m/z: calcd 294.2; found 295.2; Rt=1.121 min.

### Step 3: Synthesis of (2R)-1-benzyl-2-methyl-5-(m-tolyl)piperazine

Prepared by Scheme 1.1, step 5. Yield: 7.62 g (98.24%).

LCMS(ESI): [M]⁺ m/z: calcd 280.2; found 281.2; Rt=0.926 min.

### Step 4: Synthesis of (5R)-tert-butyl 4-benzyl-5-methyl-2-(m-tolyl)piperazine-1-carboxylate

Prepared by Scheme 1.1, step 6. Yield: 3.23 g (31.19%).

CC conditions: The crude product was purified by silica gel with EtOAc/Hexane (1:5) as an eluent mixture.

LCMS(ESI): [M]⁺ m/z: calcd 380.2; found 381.2; Rt=1.321 min.

### Step 5: Synthesis of (5R)-tert-butyl 5-methyl-2-(m-tolyl)piperazine-1-carboxylate

Prepared by Scheme 1.1, step 7. Yield: 2.52 g of crude.

LCMS(ESI): [M]⁺ m/z: calcd 290.2; found 291.2; Rt=0.960 min.

### Step 6: Synthesis of (5R)-tert-butyl 5-methyl-4-(1-methylcyclopropanecarbonyl)-2-(m-tolyl)piperazine-1-carboxylate

Prepared by Scheme 1.1, step 8A. Yield: 1.71 g of crude.

LCMS(ESI): [M-t-Bu]⁺ m/z: calcd 316.2; found 317.2; Rt=1.436 min.

### Step 7: Synthesis of ((2R)-2-methyl-5-(m-tolyl)piperazin-1-yl)(1-methylcyclopropyl)methanone

Prepared by Scheme 1.1, step 9. Yield: 1.15 g (91.81%).

LCMS(ESI): [M]⁺ m/z: calcd 272.2; found 273.2; Rt=0.715 min.

### Step 8: Synthesis of N-(6-amino-5-methylpyridin-3-yl)-2-((2S,5R)-5-methyl-4-(1-methylcyclopropanecarbonyl)-2-(m-tolyl)piperazin-1-yl)-2-oxoacetamide

Prepared by Scheme 1.1, step 10A. Yield: 98.4 mg (11.92%).

HPLC conditions: Column: SunFire C18 100*19 mm, 5 microM; 2-10 min 40-50% MeCN/water 30 ml/min; (loading pump 4 ml/min MeCN).

Compound 40: ¹H NMR (600 MHz, DMSO-d₆) δ (ppm) 0.37-0.53 (m, 2H), 0.59-0.84 (m, 2H), 1.06 (d, 3H), 1.17-1.29 (m, 2H), 2.18 (d, 3H), 2.25 (d, 3H), 2.98 (d, 1H), 3.69-4.11 (m, 2H), 4.46 (s, 1H), 4.56-5.01 (m, 1H), 5.32 (s, 1H), 7.04-7.15 (m, 3H), 7.19-7.25 (m, 1H), 7.73 (d, 2H), 7.88 (s, 1H), 8.30 (s, 1H), 10.91-11.39 (m, 1H), 13.10 (br s, 1H).

LCMS(ESI): [M]⁺ m/z: calcd 449.2; found 450.2; Rt=2.082 min.

### Example 61. Compound 35 tert-butyl (2R,5S)-2-methyl-4-[2-oxo-2-(1H-pyrazolo[4,3-c]pyridin-7-ylamino)acetyl]-5-phenyl-piperazine-1-carboxylate

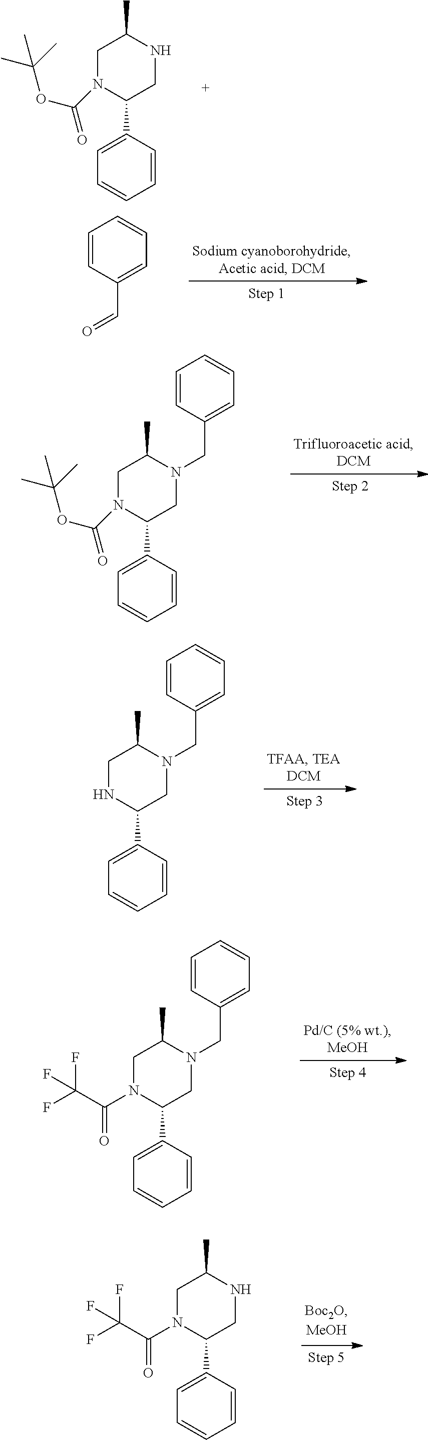

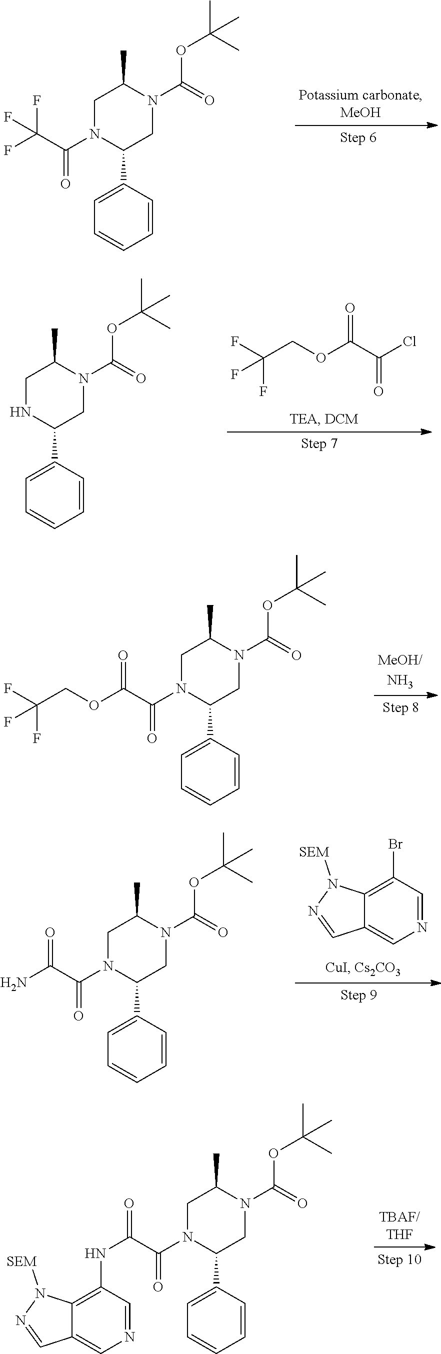

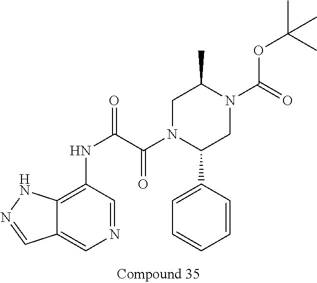 [SMILES: C[C@@H]1CN(C(=O)C(=O)Nc2cncc3cn[nH]c23)[C@@H](c2ccccc2)CN1C(=O)OC(C)(C)C]

### Step 1: tert-butyl (2S,5R)-4-benzyl-5-methyl-2-phenyl-piperazine-1-carboxylate

tert-Butyl (2S,5R)-5-methyl-2-phenyl-piperazine-1-carboxylate (0.5 g, 1.81 mmol) was dissolved in DCM (10 mL) and Acetic acid (108.64 mg, 1.81 mmol, 103.57 L) was added thereto followed by addition of benzaldehyde (287.98 mg, 2.71 mmol). Sodium cyanoborohydride (227.38 mg, 3.62 mmol) was added to the reaction mixture and the resulting mixture was stirred overnight. Aq. NaHCO₃ solution (15 ml) was added to the reaction mixture and the resulting mixture was extracted with DCM (3*20 ml). Combined organic layers were dried over Na₂SO₄, filtered and evaporated to obtain crude tert-butyl (2S,5R)-4-benzyl-5-methyl-2-phenyl-piperazine-1-carboxylate (0.9 g, 2.46 mmol, 135.74% yield) that was directly used in the next step.

LCMS(ESI): [M+1]⁺ m/z: calcd 366.2; found 367; Rt=1.109 min.

### Step 2: (2R,5S)-1-benzyl-2-methyl-5-phenyl-piperazine

Trifluoroacetic acid, 99% (1.40 g, 12.28 mmol, 945.96 L) was added dropwise to the solution of tert-butyl (2S,5R)-4-benzyl-5-methyl-2-phenyl-piperazine-1-carboxylate (0.9 g, 2.46 mmol) in DCM (10 mL) and the reaction mixture was stirred at 25° C. for 12 hr. Then the reaction mixture was diluted with DCM(10 mL) and washed with saturated aqueous NaHCO₃ (3*30 mL), dried over Na₂SO₄, and evaporated under reduced pressure to get crude (2R,5S)-1-benzyl-2-methyl-5-phenyl-piperazine (0.73 g, 2.74 mmol, 111.60% yield) that was used in the next step without purification.

LCMS(ESI): [M+1]⁺ m/z: calcd 266.2; found 267.0; Rt=0.938 min.

### Step 3: 2,2,2-trifluoro-1-[(2S,5R)-4-benzyl-5-methyl-2-phenyl-piperazin-1-yl]ethanone

(2,2,2-trifluoroacetyl) 2,2,2-trifluoroacetate (575.58 mg, 2.74 mmol, 387.07 L) was added dropwise to the solution of (2R,5S)-1-benzyl-2-methyl-5-phenyl-piperazine (0.73 g, 2.74 mmol) and Triethylamine (831.92 mg, 8.22 mmol, 1.15 mL) in DCM (10 mL) at 25° C. The reaction mixture was stirred at 25° C. for 12 hr.

The reaction mixture was diluted with DCM(10 mL) and washed with water (3*30 mL), dried over Na₂SO₄ and evaporated under reduced pressure to afford the crude 2,2,2-trifluoro-1-[(2S,5R)-4-benzyl-5-methyl-2-phenyl-piperazin-1-yl]ethanone (0.85 g, 2.35 mmol, 85.59% yield) that was directly used in the next step without purification.

LCMS(ESI): [M+1]⁺ m/z: calcd 362.3; found 363.0; Rt=1.101 min.

### Step 4: 2,2,2-trifluoro-1-[(2S,5R)-5-methyl-2-phenyl-piperazin-1-yl]ethanone

Trifluoromethyl (2S,5R)-4-benzyl-5-methyl-2-phenyl-piperazine-1-carboxylate (0.85 g, 2.25 mmol) was dissolved in MeOH (10 mL) and Palladium, 5% on activated carbon paste, 5R437 (0.15 g, 1.41 mmol) was added thereto. The reaction mixture was evacuated and backfilled three times with hydrogen and was hydrogenated at 1 atm (balloon) at 50° C. during 12 hr. The reaction mixture was cooled to the room temperature and catalyst was filtered off and the filtrate was concentrated in vacuum to obtain crude 2,2,2-trifluoro-1-[(2S,5R)-5-methyl-2-phenyl-piperazin-1-yl]ethanone (0.4 g, 1.47 mmol, 65.40% yield) that was used in the next step.

LCMS(ESI): [M+1]⁺ m/z: calcd 272.2; found 273.2; Rt=0.841 min.

### Step 5: tert-butyl (2R,5S)-2-methyl-5-phenyl-4-(2,2,2-trifluoroacetyl)piperazine-1-carboxylate

tert-Butoxycarbonyl tert-butyl carbonate (320 mg, 1.47 mmol, 337.16 L) was added in the solution of 2,2,2-trifluoro-1-[(2S,5R)-5-methyl-2-phenyl-piperazin-1-yl]ethanone (0.4 g, 1.47 mmol) in the MeOH (10 mL) at 25° C. and reaction mixture was mixed at 25° C. for 12 hr. Then the solvent was evaporated to dryness under reduced pressure to get crude product tert-butyl (2R,5S)-2-methyl-5-phenyl-4-(2,2,2-trifluoroacetyl)piperazine-1-carboxylate (0.65 g, 1.75 mmol, 118.81% yield) that was directly used in the next step.

LCMS(ESI): [M+1]⁺ m/z: calcd 372.3; found 273.2; Rt=1.320 min.

### Step 6: The synthesis of tert-butyl (2R,5S)-2-methyl-5-phenyl-piperazine-1-carboxylate

Potassium carbonate (723 mg, 5.24 mmol, 316.04 L) was added in the solution of tert-butyl (2R,5S)-2-methyl-5-phenyl-4-(2,2,2-trifluoroacetyl)piperazine-1-carboxylate (0.65 g, 1.75 mmol) in the MeOH (10 mL) at 25° C. and this reaction mixture was mixed at 25° C. for 12 hr. Then the solvent was evaporated to dryness under reduced pressure, the residue was diluted by DCM (30 mL.), the precipitate was filtered off and the filtrate was evaporated under reduced pressure to get crude product tert-butyl (2R,5S)-2-methyl-5-phenyl-piperazine-1-carboxylate (0.25 g, 904.57 μmol, 51.82% yield), that was directly used in the next step.

LCMS(ESI): [M+1]⁺ m/z: calcd 276.3; found 277.2; Rt=0.901 min.

### Step 7: The synthesis of tert-butyl (2R,5S)-2-methyl-4-[2-oxo-2-(2,2,2-trifluoroethoxy)acetyl]-5-phenyl-piperazine-1-carboxylate

2,2,2-trifluoroethyl 2-chloro-2-oxo-acetate (68.93 mg, 361.83 mol) was added to the solution of tert-butyl (2R,5S)-2-methyl-5-phenyl-piperazine-1-carboxylate (0.1 g, 361.83 μmol) and triethylamine (109.84 mg, 1.09 mmol, 151.30 L) in the DCM (20 mL) at 25° C.

Reaction was stirred at 25° C. for 12 hr. Then the reaction mixture was diluted by DCM (10 mL.) and washed by water (3*30 mL.), the organic phase was dried over Na₂SO₄, and evaporated under reduced pressure to afford crude tert-butyl (2R,5S)-2-methyl-4-[2-oxo-2-(2,2,2-trifluoroethoxy)acetyl]-5-phenyl-piperazine-1-carboxylate (0.2 g, 464.67 mol, 128% yield) that was directly used in the next step.

LCMS(ESI): [M+1]⁺ m/z: calcd. 430.1; found 375.2; Rt=1.555 min.

### Step 8: The synthesis of tert-butyl (2R,5S)-2-methyl-4-oxamoyl-5-phenyl-piperazine-1-carboxylate

tert-Butyl (2R,5S)-2-methyl-4-[2-oxo-2-(2,2,2-trifluoroethoxy)acetyl]-5-phenyl-piperazine-1-carboxylate (0.2 g, 464.67 μmol) was dissolved in the MeOH/NH₃ (5 mL) and the reaction mixture was stirred at 25° C. for 12 hr. After completion the solvent was evaporated under reduced pressure to afford crude tert-butyl (2R,5S)-2-methyl-4-oxamoyl-5-phenyl-piperazine-1-carboxylate (0.15 g, 431.77 μmol, 92.92% yield) that was used in the next step without purification.

LCMS(ESI): [M+1]⁺ m/z: calcd. 347.4; found 291.2; Rt=0.930 min.

### Step 9: The synthesis of tert-butyl (2R,5S)-2-methyl-4-[2-oxo-2-[[1-(2-trimethylsilylethoxymethyl)pyrazolo[4,3-c]pyridin-7-yl]amino]acetyl]-5-phenyl-piperazine-1-carboxylate

tert-butyl (2R,5S)-2-methyl-4-oxamoyl-5-phenyl-piperazine-1-carboxylate (0.15 g, 431.77 μmol), 2-[(7-bromopyrazolo[4,3-c]pyridin-1-yl)methoxy]ethyl-trimethyl-silane (141.74 mg, 431.77 μmol), copper (27.44 mg, 431.77 mol), iodocopper (82.23 mg, 431.77 μmol, 14.63 μL), cesium carbonate (281.36 mg, 863.54 mol) were mixed in the dioxane (5 mL) under argon stream and (1R,2R)—N₁,N₂-dimethylcyclohexane-1,2-diamine (92.12 mg, 647.65 μmol) was added thereto. The reaction mixture was stirred at 100° C. for 12 hr. After completion the reaction mixture was cooled to the 25° C. and filtered through thin pad of silica, the filter cake was washed by dioxane (3×5 mL) and the filtrate was evaporated to dryness under reduced pressure to afford crude product that was purified by reverse phase HPLC to obtain tert-butyl (2R,5S)-2-methyl-4-[2-oxo-2-[[1-(2-trimethylsilylethoxymethyl)pyrazolo[4,3-c]pyridin-7-yl]amino]acetyl]-5-phenyl-piperazine-1-carboxylate (38.70 mg, 65.07 μmol, 15% yield) thar was used as is in the next step.

LCMS(ESI): [M+1]⁺ m/z: calcd. 594.7; found 595.6; Rt=4.149 min.

### Step 10: The synthesis of tert-butyl (2R,5S)-2-methyl-4-[2-oxo-2-(1H-pyrazolo[4,3-c]pyridin-7-ylamino)acetyl]-5-phenyl-piperazine-1-carboxylate (Compound 35)

tert-Butyl (2R,5S)-2-methyl-4-[2-oxo-2-[[1-(2-trimethylsilylethoxymethyl)pyrazolo[4,3-c]pyridin-7-yl]amino]acetyl]-5-phenyl-piperazine-1-carboxylate (38.7 mg, 65.07 μmol) was dissolved in the THF (5 mL) under argon stream, and tetrabutylammonium;fluoride (340.25 mg, 1.30 mmol, 376.80 L) (1M solution in THF) was added, and the reaction mixture was evacuated and backfilled with the argon four times and stirred at 80° C. for 144 hr. The reaction was monitored by LCMS. After completion solvent was evaporated under reduced pressure, and product was purified by two reverse phase HPLC to obtain tert-butyl (2R,5S)-2-methyl-4-[2-oxo-2-(1H-pyrazolo[4,3-c]pyridin-7-ylamino)acetyl]-5-phenyl-piperazine-1-carboxylate (0.0038 g, 8.18 mol, 12.5% yield) was obtained.

LCMS(ESI): [M+1]⁺ m/z: calcd. 464.2; found 465.2; Rt=2.690 min.

### Example 62. Compound 10 5-(2-((2S,5R)-4-isobutyryl-5-methyl-2-(3-(4-methylpiperazin-1-yl)phenyl)piperazin-1-yl)-2-oxoacetamido)-2-methoxynicotinamide

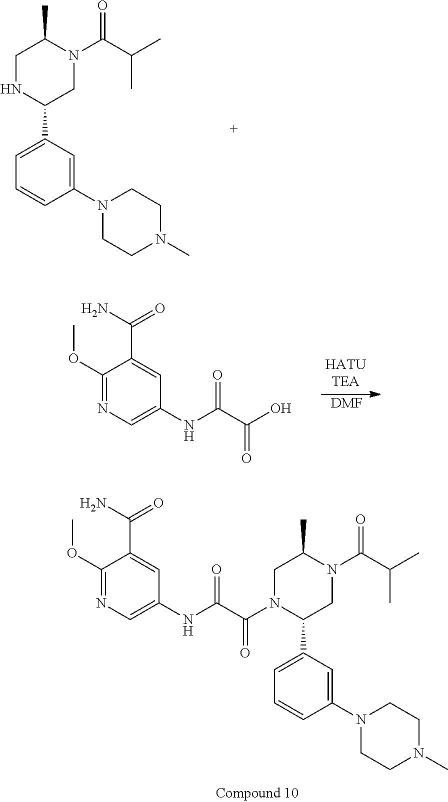

2-methyl-1-((2R,5S)-2-methyl-5-(3-(4-methylpiperazin-1-yl)phenyl)piperazin-1-yl)propan-1-one was synthesized by tert-Butyl (2S,5R)-5-methyl-2-[3-(4-methylpiperazin-1-yl)phenyl]-4-(2-methylpropanoyl)piperazine-1-carboxylate (315.00 mg, 708.49 mol) was dissolved in DCM (2 mL),TFA (2 mL) was added in one portion. The resulting mixture was allowed to stir at 20° C. for 1.5 hr. Upon completion, DCM was evaporated, 20 ml of water was added to the residue and mixture was basified with K₂CO₃ to alkaline pH. Aqueous phase was extracted with DCM (3*15 ml), combined organic layers were dried over Na₂SO₄, filtered and concentrated under reduced pressure to afford 2-methyl-1-[(2R,5S)-2-methyl-5-[3-(4-methylpiperazin-1-yl)phenyl]piperazin-1-yl]propan-1-one (180 mg, crude).

LCMS(ESI): [M]⁺ m/z: calcd 344.2; found 345.2; Rt=0.609 min.

Prepared by Scheme 1.1, step 10A. Yield: 20 mg (4.06%).

HPLC conditions: Column: SunFire C18 100*19 mm, 5 microM; 2-10 min 0-25% water-MeOH+NH₃ 30 ml/min; (loading pump 4 ml/min MeOH+NH₃).

Compound 10: ¹H NMR (600 MHz, DMSO-d₆) δ (ppm) 0.94 (m, 6H), 1.17 (m, 3H), 2.19 (m, 3H), 2.40 (m, 4H), 2.77 (m, 2H), 3.06 (m, 5H), 3.65 (m, 1H), 3.95 (m, 3H), 4.27 (m, 1H), 4.82 (m, 1H), 5.39 (m, 1H), 6.77 (m, 3H), 7.16 (m, 1H), 7.71 (s, 2H), 8.49 (m, 2H), 11.13 (m, 1H)

LCMS(ESI): [M]⁺ m/z: calcd 565.2; found 566.2; Rt=2.220 min.

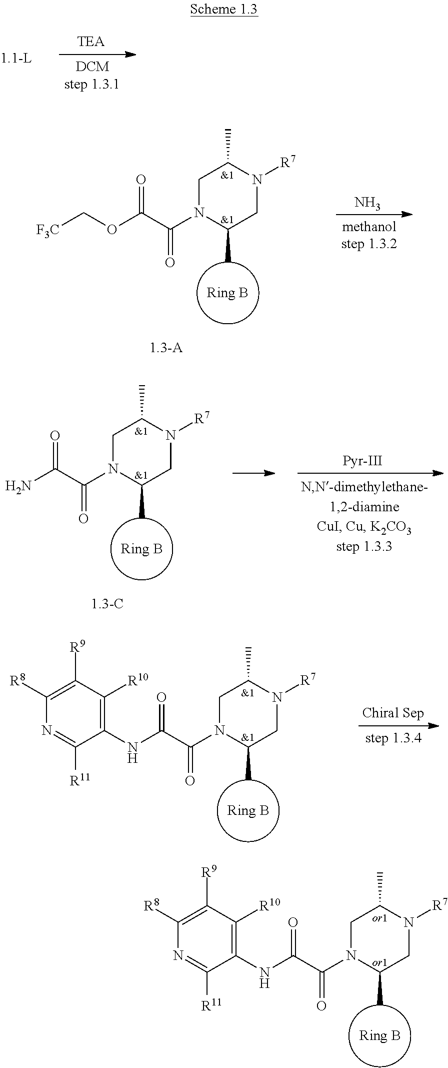

### Scheme 1.3 General Procedures

### Step 1.3.1: Synthesis of 1.3-A

The synthesis of 1.1L was described in Scheme 1.1.

1.1-L (1 eq) and TEA (1.1 eq) were dissolved in DCM and cooled to 0° C., following by the dropwise addition of 2,2,2-trifluoroethyl 2-chloro-2-oxo-acetate (1.1 eq) and the reaction mixture was stirred for 12 hr at rt. The mixture was diluted with DCM, washed with water and brine. Organic layer was dried over Na₂SO₄ and evaporated under reduced pressure to give 1.3-A which was used in the next step without further purification.

### Step 1.3.2: Synthesis of 1.3—C

1.3-A (1 eq) was dissolved in NH₃/MeOH. The reaction mixture was stirred overnight and then evaporated to dryness to give 1.3—C which was used in the next step without further purification.

### Step 1.3.3: Synthesis of racemic trans product

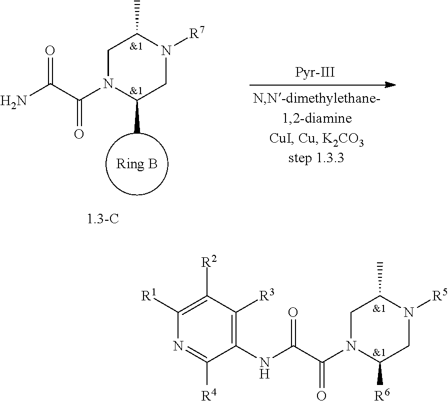

1.3—C(1 eq), Pyr-III (1 eq), Cu (1 eq), CuI (1 eq), Cs₂CO₃ (2 eq) and NN-dimethylcyclohexane-1,2-diamine (1.5 eq) were mixed in dioxane under argon, and then stirred overnight at 100° C. for 12 hr in vial. The residue was purified by HPLC to obtain pure racemic product.

### Example 63. Compound 2 (2R,5S)-tert-butyl 4-(2-((6-amino-5-(oxetan-3-yl)pyridin-3-yl)amino)-2-oxoacetyl)-5-(4-fluorophenyl)-2-methylpiperazine-1-carboxylate

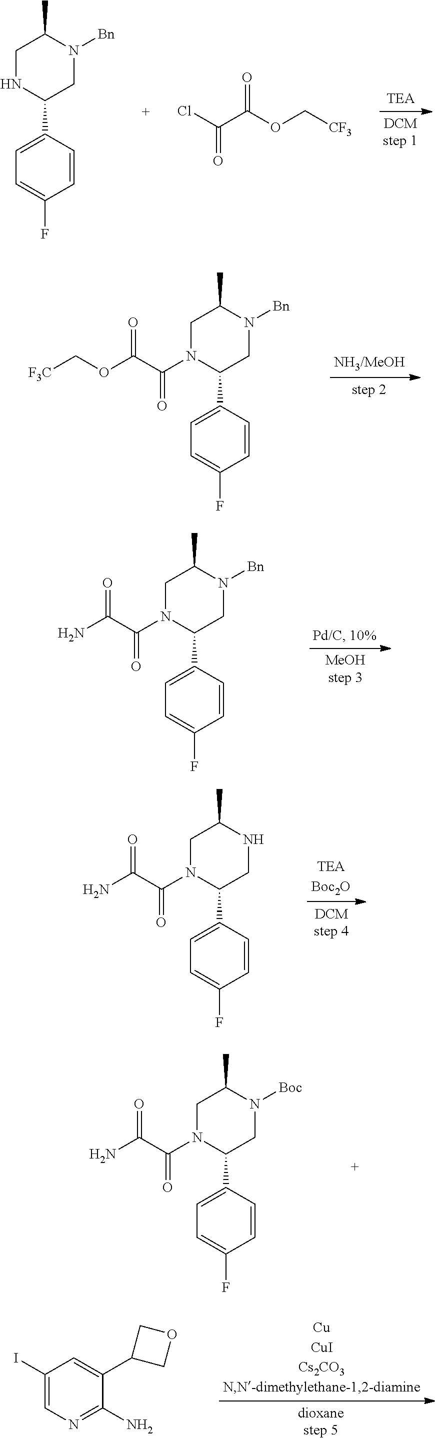

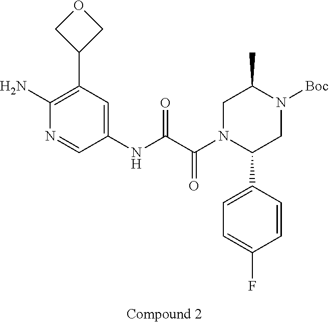 [SMILES: C[C@@H]1CN(C(=O)C(=O)Nc2cnc(N)c(C3COC3)c2)[C@@H](c2ccc(F)cc2)CN1C(=O)OC(C)(C)C]

The synthesis of (2R,5S)-1-benzyl-5-(4-fluorophenyl)-2-methylpiperazine is given by tert-Butyl (2S,5R)-4-benzyl-2-(4-fluorophenyl)-5-methyl-piperazine-1-carboxylate (700 mg, 1.82 mmol) was dissolved in DCM (4 mL) and TFA (4 mL) was added thereto. The resulting mixture was stirred for 1 hr. The reaction mixture was poured into K₂CO₃ aq. solution (1Og in 45 ml of water) and the resulting mixture was extracted with DCM (2*45 ml). Combined organic layers were dried over Na₂SO₄, filtered and concentrated in vacuum to obtain (2R,5S)-1-benzyl-5-(4-fluorophenyl)-2-methyl-piperazine (450 mg, 1.58 mmol, 86.92% yield).

### Step 1: Synthesis of 2,2,2-trifluoroethyl 2-((2S,5R)-4-benzyl-2-(4-fluorophenyl)-5-methylpiperazin-1-yl)-2-oxoacetate

Prepared by Scheme 1.3 step 1. Yield: 0.2 g of crude.

LCMS(ESI): [M]⁺ m/z: calcd 438.2; found 439.2; Rt=1.556 min.

### Step 2: Synthesis of 2-((2S,5R)-4-benzyl-2-(4-fluorophenyl)-5-methylpiperazin-1-yl)-2-oxoacetamide

Prepared by Scheme 1.3 step 2. Yield: 130 mg of crude.

LCMS(ESI): [M]⁺ m/z: calcd 355.2; found 356.2; Rt=1.006 min.

### Step 3: Synthesis of 2-((2S,5R)-2-(4-fluorophenyl)-5-methylpiperazin-1-yl)-2-oxoacetamide

The mixture of 2-oxo-2-[(2S,5R)-4-benzyl-2-(4-fluorophenyl)-5-methyl-piperazin-1-yl]acetamide (130 mg, 365.78 mol) and palladium, 10% on carbon, Type 487, dry (38.93 mg, 365.78 mol) in MeOH (5 mL) was subjected to hydrogenation (latm) at 25° C. for 36 hr. Then the mixture was filtered and evaporated to dryness affording 2-oxo-2-[(2S,5R)-2-(4-fluorophenyl)-5-methyl-piperazin-1-yl]acetamide (0.09 g, crude).

LCMS(ESI): [M]⁺ m/z: calcd 265.2; found 266.2; Rt=0.273 min.

### Step 4: Synthesis of (2R,5S)-tert-butyl 4-(2-amino-2-oxoacetyl)-5-(4-fluorophenyl)-2-methylpiperazine-1-carboxylate

2-Oxo-2-[(2S,5R)-2-(4-fluorophenyl)-5-methyl-piperazin-1-yl]acetamide (0.09 g, 339.26 mol) and TEA (51.49 mg, 508.89 mol, 70.93 L) was dissolved in DCM (5 mL), then di-tert-butyl dicarbonate (81.45 mg, 373.19 mol, 85.64 L) was added dropwise under ice/water cooling, after that the reaction mixture was stirred at 26° C. for 14 hr.Then the mixture was evaporated to dryness affording tert-butyl (2R,5S)-5-(4-fluorophenyl)-2-methyl-4-oxamoyl-piperazine-1-carboxylate (110 mg, 301.04 mol, 88.73% yield).

LCMS(ESI): [M-Boc]⁺ m/z: calcd 265.2; found 266.2; Rt=1.258 min.

### Step 5: Synthesis of (2R,5S)-tert-butyl 4-(2-((6-amino-5-(oxetan-3-yl)pyridin-3-yl)amino)-2-oxoacetyl)-5-(4-fluorophenyl)-2-methylpiperazine-1-carboxylate (Compound 2)

Prepared by Scheme 1.3 step 3A. Yield: 19.4 mg (13.80%).

HPLC conditions: Column: SunFire C18 100*19 mm, 5 microM; 2-10 min 10-40% MeCN 30 ml/min; (loading pump 4 ml/min MeCN).

Compound 2: ¹H NMR (600 MHz, DMSO-d₆) δ (ppm) 1.11-1.18 (m, 3H), 1.30-1.37 (m, 9H), 2.79-3.25 (m, 1H), 3.35-3.53 (m, 1H), 3.59-3.98 (m, 1H), 4.02-4.17 (m, 1H), 4.19-4.24 (m, 1H), 4.32-4.42 (m, 1H), 4.48-4.56 (m, 2H), 4.87-4.95 (m, 2H), 5.25-5.58 (m, 1H), 5.58-5.67 (m, 2H), 7.16-7.26 (m, 2H), 7.30-7.43 (m, 2H), 7.60-7.78 (m, 1H), 8.02-8.15 (m, 1H), 10.55-10.69 (m, 1H).

LCMS(ESI): [M]⁺ m/z: calcd 513.2; found 514.2; Rt=2.501 min.

### Example 64. Compound 156 2-((2S,5R)-5-methyl-2-phenyl-4-pivaloylpiperazin-1-yl)-2-oxo-N-(1H-pyrazolo[4,3-c]pyridin-7-yl)acetamide

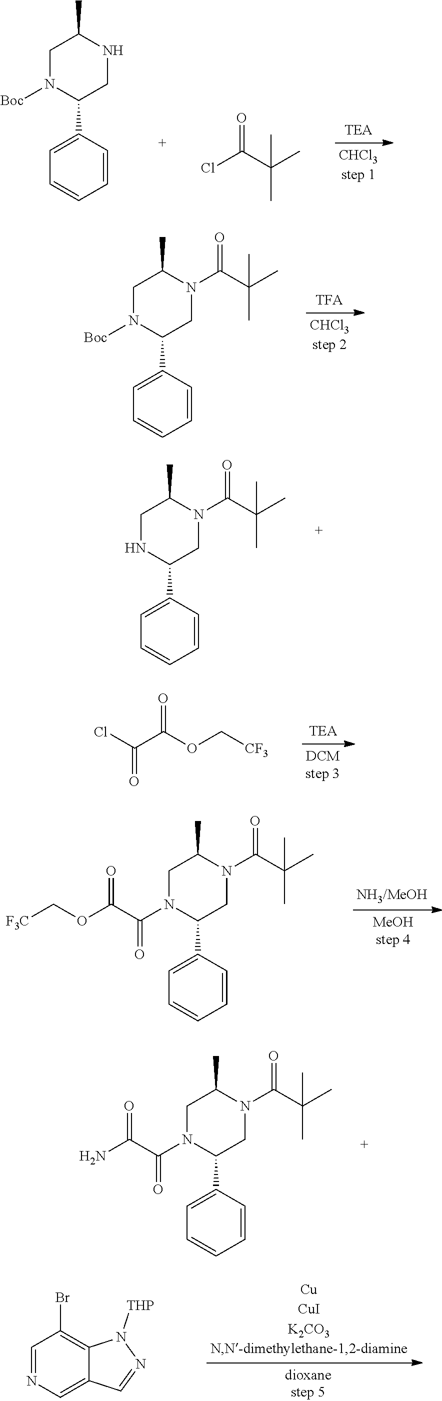

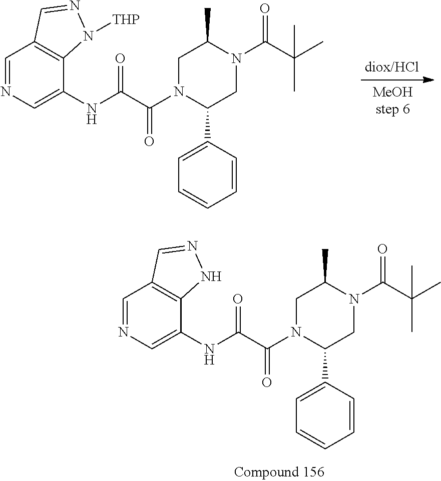

### Step 1: Synthesis of (2S,5R)-tert-butyl 5-methyl-2-phenyl-4-pivaloylpiperazine-1-carboxylate

The synthesis of (2S,5R)-tert-butyl 5-methyl-2-phenylpiperazine-1-carboxylate is given by the following procedure. (2R)-1-Benzyl-2-methyl-5-phenyl-piperazine (2.1 g, 7.88 mmol) was dissolved in MeOH (50 mL), tert-butoxycarbonyl tert-butyl carbonate (2.06 g, 9.46 mmol, 2.17 mL) was added dropwise to the resulting mixture. The reaction mixture was stirred overnight. After completion, the reaction mixture was concentrated in vacuum. The residue was diluted with water (20 ml) and the resulting mixture was extracted with CHCl₃ (2*20 ml). Combined organic layers were washed with brine (25 ml), dried over Na₂SO₄, filtered and evaporated. The residue was purified by reverse phase HPLC chromatography (Column: Chromatorex 18 SMB 100-5T 100*19 mm, 5 microM; 0-6 min 20-50% water-MeCN 30 ml/min (loading pump 4 ml MeCN)) to give tert-butyl (2S,5R)-4-benzyl-5-methyl-2-phenyl-piperazine-1-carboxylate (0.75 g, 2.05 mmol, 25.96% yield).

LCMS(ESI): [M]⁺ m/z: calcd 366.2; found 367.2; Rt=3.202 min.

Prepared by Scheme 1.4, step 7A. Yield: 0.65 g (99.67%).

LCMS(ESI): [M-t-Bu]⁺ m/z: calcd 304.2; found 305.2; Rt=1.597 min.

### Step 2: Synthesis of 2,2-dimethyl-1-((2R,5S)-2-methyl-5-phenylpiperazin-1-yl)propan-1-one

Prepared by Scheme 1.4 step 8. Yield: 0.46 g (97.98%).

LCMS(ESI): [M]⁺ m/z: calcd 260.2; found 261.2; Rt=0.910 min.

### Step 3: Synthesis of 2,2,2-trifluoroethyl 2-((2S,5R)-5-methyl-2-phenyl-4-pivaloylpiperazin-1-yl)-2-oxoacetate

Prepared by Scheme 1.3, step 1. Yield: 0.45 g (94.24%).

LCMS(ESI): [M]⁺ m/z: calcd 414.2; found 415.2; Rt=1.451 min.

### Step 4: Synthesis of 2-((2S,5R)-5-methyl-2-phenyl-4-pivaloylpiperazin-1-yl)-2-oxoacetamide

Prepared by Scheme 1.3, step 2. Yield: 0.35 mg (97.26%).

LCMS(ESI): [M]⁺ m/z: calcd 331.2; found 332.2; Rt=1.136 min.

### Step 5: Synthesis of 2-((2S,5R)-5-methyl-2-phenyl-4-pivaloylpiperazin-1-yl)-2-oxo-N-(1-(tetrahydro-2H-pyran-2-yl)-1H-pyrazolo[4,3-c]pyridin-7-yl)acetamide

Prepared by Scheme 1.3, step 3A. Yield: 0.32 g of crude.

LCMS(ESI): [M]⁺ m/z: calcd 532.2; found 533.2; Rt=1.119 min.

### Step 6: Synthesis of 2-((2S,5R)-5-methyl-2-phenyl-4-pivaloylpiperazin-1-yl)-2-oxo-N-(1H-pyrazolo[4,3-c]pyridin-7-yl)acetamide (Compound 156)

The solution of 2-[(2S,5R)-4-(2,2-dimethylpropanoyl)-5-methyl-2-phenyl-piperazin-1-yl]-2-oxo-N-(1-tetrahydropyran-2-ylpyrazolo[4,3-c]pyridin-7-yl)acetamide (0.32 g, 600.79 umol) in MeOH (10 mL) and diox/HCl (10 mL) was stirred at 25° C. for 16 hr. Upon completion, the reaction mixture was concentrated under reduced pressure to obtain crude product. The obtained crude product was purified by reverse phase HPLC chromatography to afford product 2-[(2S,5R)-4-(2,2-dimethylpropanoyl)-5-methyl-2-phenyl-piperazin-1-yl]-2-oxo-N-(1H-pyrazolo[4, 3-c]pyridin-7-yl)acetamide (113 mg, 251.94 umol, 41.94% yield).

Compound 156:¹H NMR (600 MHz, DMSO-d₆) δ (ppm) 1.09 (m, 9H), 1.28 (m, 3H), 1.62 (m, 2H), 1.82 (m, 1H), 1.99 (m, 1H), 4.00 (m, 1H), 4.43 (m, 1H), 4.92 (s, 1H), 5.67 (s, 1H), 7.34 (m, 5H), 8.34 (m, 1H), 8.46 (m, 1H), 8.95 (m, 1H).

LCMS(ESI): [M]⁺ m/z: calcd 448.2; found 449.2; Rt=2.771 min.

### Example 65. Compound 127 N-(6-amino-5-(oxetan-3-yl)pyridin-3-yl)-2-((2S,5R)-5-methyl-2-phenyl-4-pivaloylpiperazin-1-yl)-2-oxoacetamide

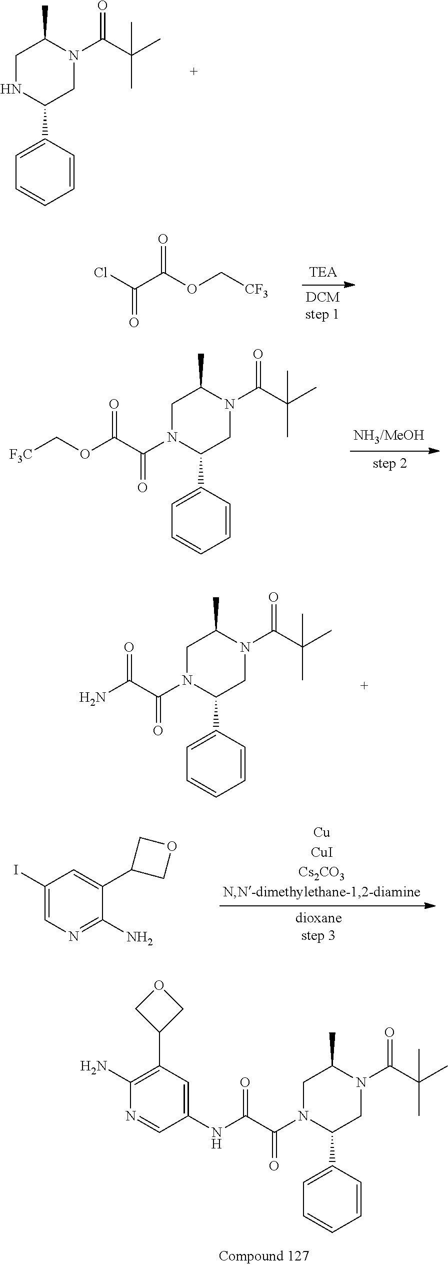

### Step 1: Synthesis of 2,2,2-trifluoroethyl 2-((2S,5R)-5-methyl-2-phenyl-4-pivaloylpiperazin-1-yl)-2-oxoacetate

2,2-dimethyl-1-((2R,5S)-2-methyl-5-phenylpiperazin-1-yl)propan-1-one ispPrepared by Scheme 1.1 step 9. Yield: 0.46 g (97.98%).

LCMS(ESI): [M]⁺ m/z: calcd 260.2; found 261.2; Rt=0.910 min.

Prepared by Scheme 1.3 step 1. Yield: 320 mg of crude.

LCMS(ESI): [M]⁺ m/z: calcd 414.2; found 415.2; Rt=1.398 min.

### Step 2: Synthesis of 2-((2S,5R)-5-methyl-2-phenyl-4-pivaloylpiperazin-1-yl)-2-oxoacetamide

Prepared by Scheme 1.3 step 2. Yield: 243 mg of crude.

LCMS(ESI): [M]⁺ m/z: calcd 331.2; found 332.2; Rt=1.123 min.

### Step 3: Synthesis of N-(6-amino-5-(oxetan-3-yl)pyridin-3-yl)-2-((2S,5R)-5-methyl-2-phenyl-4-pivaloylpiperazin-1-yl)-2-oxoacetamide (Compound 127)

The synthesis of 5-iodo-3-(oxetan-3-yl)pyridin-2-amine is given by Intermediate 2.

Prepared by Scheme 1.3 step 3A. Yield: 11 mg (6.33%).

HPLC conditions: Column: SunFire C18 100*19 mm, 5 microM; 2-10 min 0-80% MeCN+NH₃; (loading pump 4 ml/min MeCN).

Compound 127: ¹H NMR (600 MHz, DMSO-d₆) δ (ppm) 1.08 (s, 9H), 1.24 (s, 3H), 2.62-3.20 (m, 2H), 3.65-4.06 (m, 1H), 4.17-4.25 (m, 1H), 4.42-4.57 (m, 3H), 4.69-5.08 (m, 3H), 5.34-5.67 (m, 3H), 7.23-7.29 (m, 2H), 7.29-7.38 (m, 3H), 7.69-7.77 (m, 1H), 8.07-8.15 (m, 1H), 10.55 (s, 1H).

LCMS(ESI): [M]⁺ m/z: calcd 479.2; found 480.2; Rt=0.980 min.

### Example 66. Compound 55 2-((2S,5R)-5-methyl-2-phenyl-4-pivaloylpiperazin-1-yl)-2-oxo-N-(1H-pyrazolo[3,4-c]pyridin-4-yl)acetamide

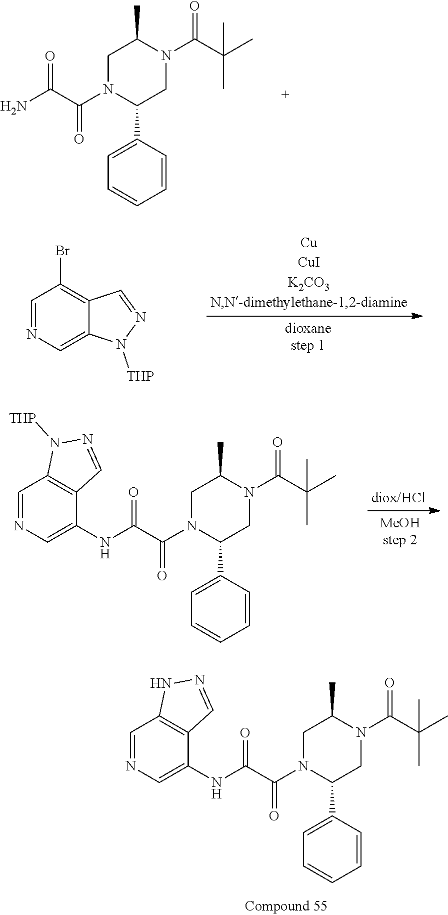

### Step 1: Synthesis of 2-((2S,5R)-5-methyl-2-phenyl-4-pivaloylpiperazin-1-yl)-2-oxo-N-(1-(tetrahydro-2H-pyran-2-yl)-1H-pyrazolo[3,4-c]pyridin-4-yl)acetamide

The synthesis of 2-((2S,5R)-5-methyl-2-phenyl-4-pivaloylpiperazin-1-yl)-2-oxoacetamide is prepared by Scheme 1.3 step 2. Yield: 0.35 mg (97.26%).

LCMS(ESI): [M]⁺ m/z: calcd 331.2; found 332.2; Rt=1.136 min.

Prepared by Scheme 1.3, Step 3A. Yield: 0.32 g of crude.

LCMS(ESI): [M]⁺ m/z: calcd 532.2; found 533.2; Rt=1.297 min.

### Step 2: Synthesis of 2-((2S,5R)-5-methyl-2-phenyl-4-pivaloylpiperazin-1-yl)-2-oxo-N-(1H-pyrazolo[3,4-c]pyridin-4-yl)acetamide (Compound 55)

The solution of 2-[(2S,5R)-4-(2,2-dimethylpropanoyl)-5-methyl-2-phenyl-piperazin-1-yl]-2-oxo-N-(1-tetrahydropyran-2-ylpyrazolo[3,4-c]pyridin-4-yl)acetamide (0.32 g, 600.79 umol) in MeOH (10 mL) and diox/HCl (10 mL) was stirred at 25° C. for 16 hr. Upon completion, the reaction mixture was concentrated under reduced pressure to obtain crude product. The obtained crude product was purified by reverse phase HPLC chromatography to afford product 2-[(2S,5R)-4-(2,2-dimethylpropanoyl)-5-methyl-2-phenyl-piperazin-1-yl]-2-oxo-N-(1H-pyrazolo[3, 4-c]pyridin-4-yl)acetamide (0.034 g, 75.81 umol, 12.62% yield).

Compound 55: ¹H NMR (600 MHz, DMSO-d₆) δ (ppm) 1.07 (s, 9H), 1.26-1.33 (m, 3H), 2.63-3.24 (m, 1H), 3.33-3.46 (m, 1H), 3.55-4.09 (m, 1H), 4.42-4.56 (m, 1H), 4.64-5.25 (m, 1H), 5.25-5.75 (m, 1H), 7.21-7.33 (m, 3H), 7.36-7.40 (m, 2H), 8.21-8.34 (m, 1H), 8.51-8.64 (m, 1H), 8.77-8.87 (m, 1H), 11.22-11.52 (m, 1H), 13.53-13.83 (m, 1H).

LCMS(ESI): [M]⁺ m/z: calcd 448.2; found 449.2; Rt=2.993 min.

### Example 67. Compound 72 N-(6-amino-5-(oxetan-3-yl)pyridin-3-yl)-2-((2S,5R)-2-(4-fluorophenyl)-5-methyl-4-(1-(trifluoromethyl)cyclopropanecarbonyl)piperazin-1-yl)-2-oxoacetamide

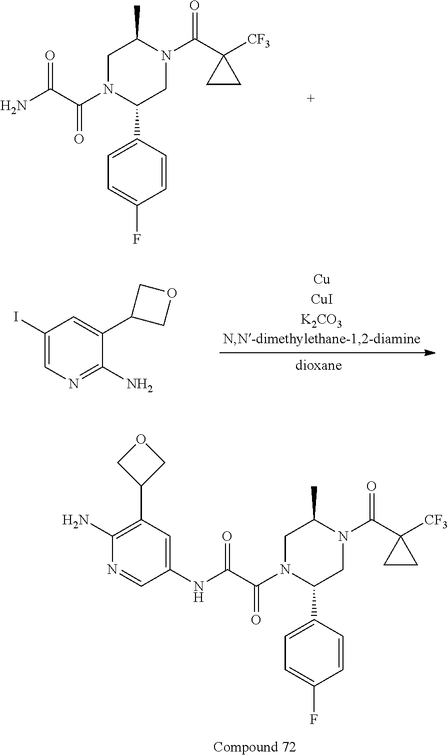

The synthesis of 2-((2S,5R)-2-(4-fluorophenyl)-5-methyl-4-(1-(trifluoromethyl)cyclopropanecarbonyl)piperazin-1-yl)-2-oxoacetamide is given by the following procedure. 2,2,2-Trifluoroethyl 2-chloro-2-oxo-acetate (917.86 mg, 4.82 mmol) was added dropwise to the solution of [(2R,5S)-5-(4-fluorophenyl)-2-methyl-piperazin-1-yl]-[1-(trifluoromethyl)cyclopropyl]methanone (1.78 g, 4.02 mmol, TFA) and TEA (1.22 g, 12.05 mmol, 1.68 mL) in DCM (20 mL). After addition was complete, resulting mixture was stirred at ambient temperature for one hour. Then, ammonia (7N solution in MeOH) (11.68 g, 104.98 mmol, 15 mL, 15.3% purity) was added and obtained solution was stirred at 20° C. for 16 hr. Volatiles were removed under reduced pressure and residue was partitioned between 10% aq. K₂CO₃ solution (30 ml) and EtOAc (50 ml). Organic layer was separated, dried over Na₂SO₄ and concentrated in vacuum, affording 2-[(2S,5R)-2-(4-fluorophenyl)-5-methyl-4-[1-(trifluoromethyl)cyclopropanecarbonyl]piperazin-1-yl]-2-oxo-acetamide (1.78 g, crude).

LCMS(ESI): [M]⁺ m/z: calcd 401.2; found 402.2; Rt=1.167 min.

The synthesis of 5-iodo-3-(oxetan-3-yl)pyridin-2-amine by Intermediate 2.

Prepared by general procedure scheme 1.3 step 3A. Yield: 7.9 mg (5.67%).

HPLC conditions: Column: SunFire 100*19 mm, 5 microM; 2-10 min 40-65% MeCN/water 30 ml/min; (loading pump 4 ml/min MeCN).

Compound 72: ¹H NMR (DMSO-d₆, 600 MHz): δ (ppm) 0.98-1.28 (m, 6H), 3.06-3.08 (m, 1H), 3.73-4.55 (m, 6H), 4.90-4.94 (m, 3H), 5.43-5.77 (m, 3H), 6.92-7.39 (m, 4H), 7.74-7.79 (m, 1H), 8.12-8.15 (m, 1H), 10.67 (m, 1H).

LCMS(ESI): [M]⁺ m/z: calcd 549.2; found 550.2; Rt=2.719 min.

### Example 68. Compound 147 2-((2S,5R)-5-methyl-4-(1-methylcyclopropanecarbonyl)-2-phenylpiperazin-1-yl)-2-oxo-N-(1H-pyrazolo[3,4-c]pyridin-4-yl)acetamide

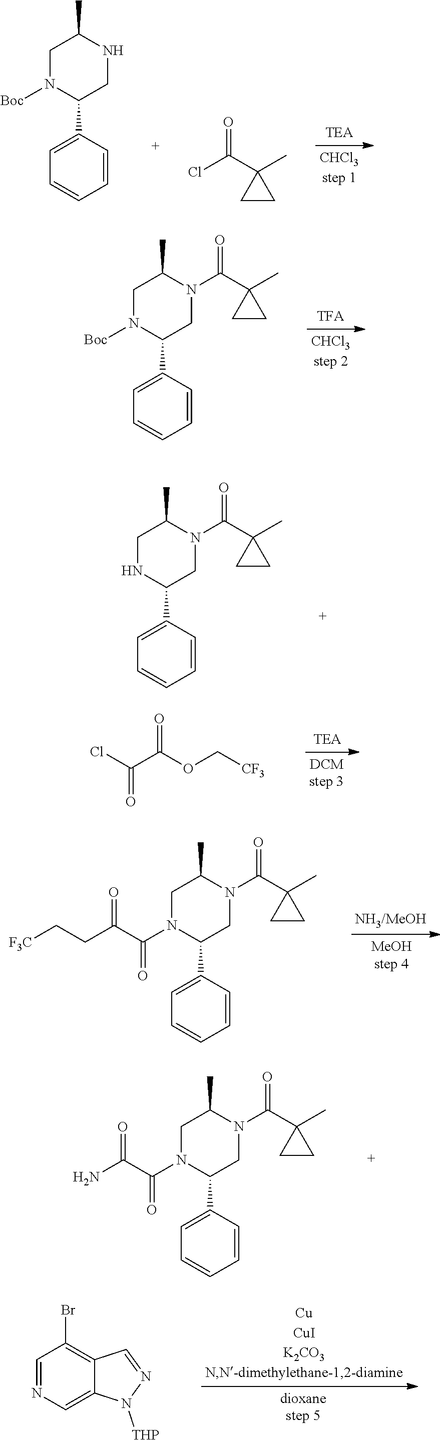

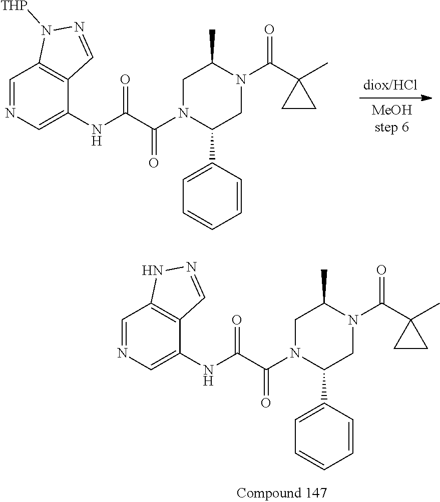

### Step 1: Synthesis of (2S,5R)-tert-butyl 5-methyl-4-(1-methylcyclopropanecarbonyl)-2-phenylpiperazine-1-carboxylate

The synthesis of (2S,5R)-tert-butyl 5-methyl-2-phenylpiperazine-1-carboxylate is Prepared as described in Scheme 1.1, step 1.1.7. Yield: 0.39 g (97.58%). LCMS(ESI): [M]⁺ m/z: calcd 276.2; found 277.2; Rt=0.997 min.

Prepared by Scheme 1.4, step 7A. Yield: 0.45 g (99.13%).

LCMS(ESI): [M]⁺ m/z: calcd 358.2; found 359.2; Rt=1.312 min.

### Step 2: Synthesis of ((2R,5S)-2-methyl-5-phenylpiperazin-1-yl)(1-methylcyclopropyl)methanone

Prepared by Scheme 1.4, step 8. Yield: 0.3 g (92.5%).

LCMS(ESI): [M]⁺ m/z: calcd 258.2; found 259.2; Rt=0.820 min.

### Step 3: Synthesis of 2,2,2-trifluoroethyl 2-((2S,5R)-5-methyl-4-(1-methylcyclopropanecarbonyl)-2-phenylpiperazin-1-yl)-2-oxoacetate

Prepared by Scheme 1.3 step 1. Yield: 0.47 g (98.15%).

LCMS(ESI): [M]⁺ m/z: calcd 412.2; found 413.2; Rt=1.223 min.

### Step 4: Synthesis of 2-((2S,5R)-5-methyl-4-(1-methylcyclopropanecarbonyl)-2-phenylpiperazin-1-yl)-2-oxoacetamide

Prepared by Scheme 1.3 step 2. Yield: 0.37 mg (98.56%).

LCMS(ESI): [M]⁺ m/z: calcd 329.2; found 330.2; Rt=1.025 min.

### Step 5: Synthesis of 2-((2S,5R)-5-methyl-4-(1-methylcyclopropanecarbonyl)-2-phenylpiperazin-1-yl)-2-oxo-N-(1-(tetrahydro-2H-pyran-2-yl)-1H-pyrazolo[3,4-c]pyridin-4-yl)acetamide

Prepared by Scheme 1.3 step 3A. Yield: 0.4 g of crude.

LCMS(ESI): [M]⁺ m/z: calcd 530.2; found 531.2; Rt=1.224 min.

### Step 6: Synthesis of 2-((2S,5R)-5-methyl-4-(1-methylcyclopropanecarbonyl)-2-phenylpiperazin-1-yl)-2-oxo-N-(1H-pyrazolo[3,4-c]pyridin-4-yl)acetamide (Compound 147)

The solution of 2-[(2S,5R)-5-methyl-4-(1-methylcyclopropanecarbonyl)-2-phenyl-piperazin-1-yl]-2-oxo-N-(1-tetrahydropyran-2-ylpyrazolo[3,4-c]pyridin-4-yl)acetamide (0.4 g, 753.84 umol) in MeOH (10 mL) and diox/HCl (10 mL) was stirred at 25° C. for 16 hr. Upon completion, the reaction mixture was concentrated under reduced pressure to obtain crude product. The obtained crude product was purified by reverse phase HPLC chromatography to afford product 2-[(2S,5R)-5-methyl-4-(1-methylcyclopropanecarbonyl)-2-phenyl-piperazin-1-yl]-2-oxo-N-(1H-pyrazolo[3,4-c]pyridin-4-yl)acetamide (20 mg, 44.79 mol, 5.94% yield).

Compound 147: ¹H NMR (600 MHz, DMSO-d₆) δ (ppm) 0.48-0.80 (m, 2H), 1.04 (s, 3H), 1.22-1.31 (m, 2H), 2.75-2.95 (m, 2H), 3.63-3.64 (m, 1H), 4.12-4.14 (m, 1H), 4.48-4.57 (m, 2H), 4.87-4.95 (m, 1H), 5.30-5.33 (m, 1H), 5.72-5.79 (m, 1H), 7.26-7.39 (m, 5H), 8.33 (s, 1H), 8.60 (s, 1H), 8.85 (s, 1H), 11.42 (m, 1H), 13.72 (m, 1H).

LCMS(ESI): [M]⁺ m/z: calcd 446.2; found 447.2; Rt=2.297 min.

### Example 69. Compound 100 2-((2S,5R)-5-methyl-2-phenyl-4-(1-(trifluoromethyl)cyclopropanecarbonyl)piperazin-1-yl)-2-oxo-N-(1H-pyrazolo[4,3-c]pyridin-7-yl)acetamide

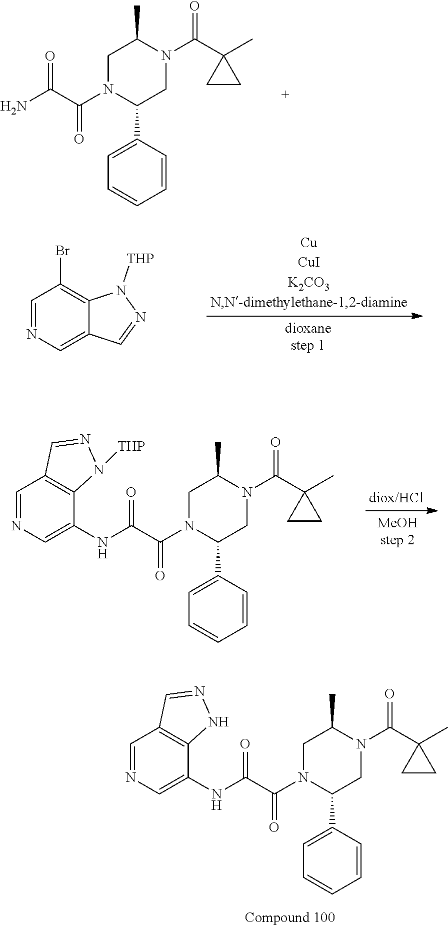

### Step 1: Synthesis of 2-((2S,5R)-5-methyl-2-phenyl-4-(1-(trifluoromethyl)cyclopropanecarbonyl)piperazin-1-yl)-2-oxo-N-(1-(tetrahydro-2H-pyran-2-yl)-1H-pyrazolo[4,3-c]pyridin-7-yl)acetamide

The synthesis of 2-((2S,5R)-5-methyl-2-phenyl-4-(1-(trifluoromethyl)cyclopropanecarbonyl) piperazin-1-yl)-2-oxoacetamide is prepared by Scheme 1.3 step 2. Yield: 0.47 mg (98.58%).

LCMS(ESI): [M]⁺ m/z: calcd 383.2; found 384.2; Rt=1.139 min

Prepared by Scheme 1.3, step 3A. Yield: 0.35 g of crude.

LCMS(ESI): [M]⁺ m/z: calcd 584.2; found 585.2; Rt=1.214 min.

### Step 2: Synthesis of 2-((2S,5R)-5-methyl-2-phenyl-4-(1-(trifluoromethyl)cyclopropanecarbonyl)piperazin-1-yl)-2-oxo-N-(1H-pyrazolo[4,3-c]pyridin-7-yl)acetamide (Compound 100)

The solution of 2-[(2S,5R)-5-methyl-2-phenyl-4-[1-(trifluoromethyl)cyclopropanecarbonyl]piperazin-1-yl]-2-oxo-N-(1-tetrahydropyran-2-ylpyrazolo[4,3-c]pyridin-7-yl)acetamide (0.35 g, 598.71 umol) in MeOH (5 mL) and diox/HCl (5 mL) was stirred at 25° C. for 16 hr. Upon completion, the reaction mixture was concentrated under reduced pressure to obtain crude product. The obtained crude product was purified by reverse phase HPLC chromatography to afford product 2-[(2S,5R)-5-methyl-2-phenyl-4-[1-(trifluoromethyl)cyclopropanecarbonyl]piperazin-1-yl]-2-oxo-N-(1H-pyrazolo[4,3-c]pyridin-7-yl)acetamide (36 mg, 71.93 umol, 12.01% yield).

Compound 100: ¹H NMR (600 MHz, DMSO-d₆) δ (ppm) 1.19 (m, 7H), 2.85 (m, 1H), 4.00 (m, 3H), 4.49 (m, 1H), 4.97 (m, 1H), 5.83 (m, 1H), 7.33 (m, 4H), 8.40 (m, 2H), 8.98 (s, 1H), 11.28 (m, 1H), 13.08 (m, 1H).

LCMS(ESI): [M]⁺ m/z: calcd 500.2; found 501.2; Rt=2.728 min.

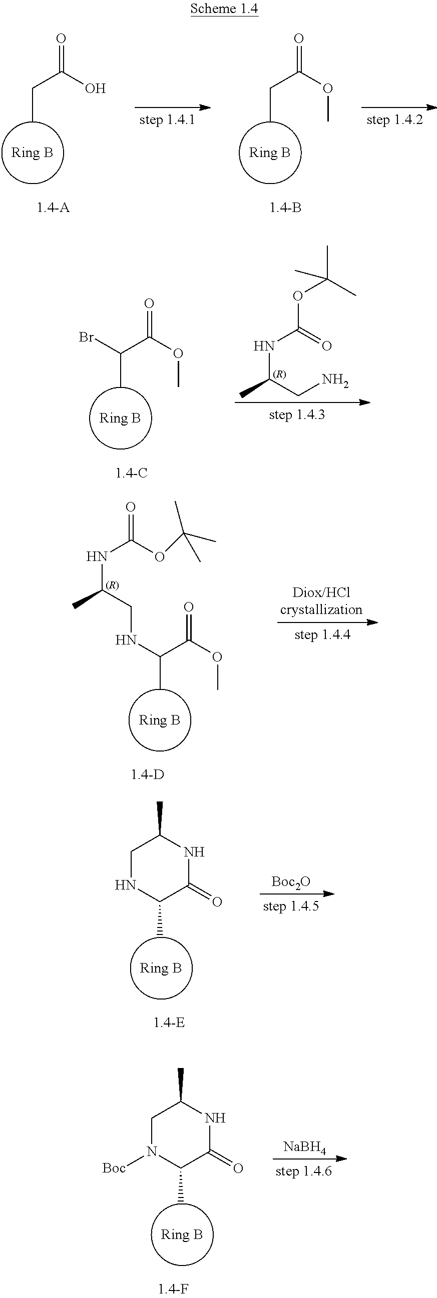

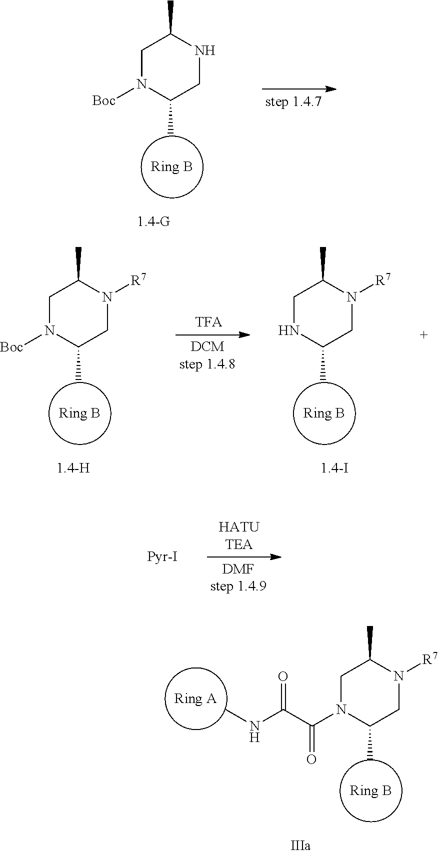

### Scheme 1.4 General Procedures

### Step 1.4.3: Synthesis of 10.4-D

1.1-C(1 eq) was added dropwise to the solution of tert-butyl N-[(1R)-2-amino-1-methyl-ethyl]carbamate (1 eq) and TEA (2 eq) in MeCN. Resulting reaction mixture was stirred at 20° C. for 14 hr. Then, volatiles were removed under reduced pressure and residue was partitioned between 10% aq. K₂CO₃ solution and MTBE. Organic layer was separated, dried over Na₂SO₄ and concentrated under reduced pressure to obtain 1.1-D which was used further without purification.

### Step 1.4.4: Synthesis of 1.4-E

Hydrogen chloride solution 4.0 M in dioxane (4 eq) was added portion wise to a solution of 1.4-D (1 eq) in MeOH. Resulting mixture was stirred at 20° C. for 14 hr. Then, volatiles were removed under reduced pressure and residue was partitioned between 25% aq. K₂CO₃ solution and DCM. Organic layer was separated, and aqueous layer was extracted with DCM twice. Combined DCM layers were dried over Na₂SO₄ and concentrated in vacuum. After solvent was distilled off, residue was heated to 80° C. under reduced pressure (approx. 15 torr) for 1 hour. Then, it was dissolved in boiling toluene. Resulting solution was leaved for crystallization at 20° C. for 6 hours. Obtained white crystals were filtered on and dried, affording 1.4-E.

### Step 1.4.5: Synthesis of 1.4-F

Boc₂O (1.15 eq) was added dropwise to the solution of 1.4-E and TEA (1.2 eq) in DCM. After addition was complete, resulting mixture was stirred at 20° C. for 4 hr. Then, volatiles were removed under reduced pressure and residue was triturated with cold hexane (60 ml). Resulting precipitate was filtered on and dried, affording 1.4-F.

### Step 1.4.6: Synthesis of 1.4-G

Solution of 1.4-F (1 eq) and acetic acid (10 eq) in dioxane was added dropwise to a refluxed solution of NaBH4 (10 eq) in dioxane. Reaction mixture was refluxed for 3 hr. After then it was cooled to rt, quenched with water and extracted with EtOAc. Organic layer was separated, dried over Na₂SO₄ and evaporated to give a crude. It was purified by flash chromatography to afford 1.4-G.

### Step 1.4.7A: Synthesis of 1.4-H

(R⁵)₂O (1 eq) or R⁵Cl (1.3 eq) was added dropwise to a solution of 1.4-G (1 eq) and TEA (3 eq) in DCM. After addition was complete, resulting mixture was stirred at 20° C. for 3 hr. Then, 15% aq. K₂CO₃ solution was added and stirring was continued for 10 min. After that, organic layer was separated, dried over Na₂SO₄, filtered and evaporated to obtain 1.4-H.

### Step 1.4.8: Synthesis of 1.4—I

1.4-H (1 eq) was dissolved in DCM and TFA (7 eq) was added thereto. The resulting mixture was stirred at 25° C. for 15 hr, and then evaporated in vacuum to afford 1.4—I (TFA-salt).

### Step 1.4.9A: Synthesis of IIIa

1.4—I (1 eq), Pyr-I (1 eq) and TEA (2.5 eq+1.0 eq per each acid eq, if amine salt used) were mixed together in DMF. HATU (1.5 eq) was added thereto and the resulting mixture was stirred overnight. The reaction mixture was concentrated in vacuum and the residue was purified by HPLC to obtain the product.

### Example 70. Compound 139 N-(6-amino-5-ethylpyridin-3-yl)-2-((2S,5R)-2-(3-chloro-4-fluorophenyl)-5-methyl-4-(1-(trifluoromethyl)cyclopropanecarbonyl)piperazin-1-yl)-2-oxoacetamide

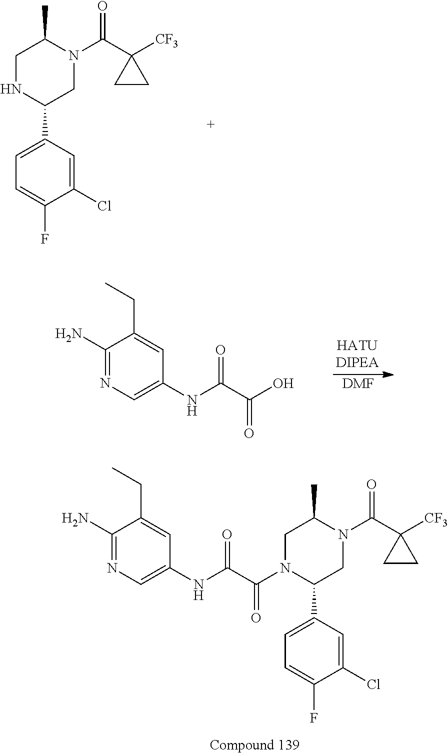

The synthesis of ((2R)-5-(3-chloro-4-fluorophenyl)-2-methylpiperazin-1-yl)(1-(trifluoromethyl)cyclopropyl)methanone is given by tert-Butyl (2S,5R)-2-(3-chloro-4-fluoro-phenyl)-5-methyl-4-[1-(trifluoromethyl)cyclopropane carbonyl]piperazine-1-carboxylate (0.29 g, 623.82 μmol) was dissolved in DCM (30.23 mL) and TFA (355.65 mg, 3.12 mmol, 240.30 L) was added to reaction mixture at 0° C. then the solution was allowed to warm to 25° C. and stirred at 25° C. for 16 hr. Reaction mixture was diluted with DCM (30 ml.) and washed by saturated aqueous NaHCO₃ (3*30 ml). The resulting organic phase was dried over Na₂SO₄ and evaporated under reduced pressure to afford [(2R,5S)-5-(3-chloro-4-fluoro-phenyl)-2-methyl-piperazin-1-yl]-[1-(trifluoromethyl)cyclopropyl]methanone (0.18 g, 493.47 μmol, 79.10% yield).

LCMS(ESI): [M]⁺ m/z: calcd 364.2; found 365.2; Rt=1.044 min.

Prepared by Scheme 1.4, step 9B. Yield: 43.1 mg (16.63%).

HPLC conditions: Column: Chromatorex C18 100*19 mm, 5 microM; 0-6 min 10-50% MeCN-water+0.2% FA; (loading pump 4 ml/min MeCN).

Compound 139: ¹H NMR (600 MHz, DMSO-d₆) δ (ppm) 0.99-1.07 (m, 1H), 1.13-1.33 (m, 10H), 2.64-3.09 (m, 1H), 3.82-5.81 (m, 4H), 7.19-7.45 (m, 3H), 7.46-7.67 (m, 3H), 7.82-7.91 (m, 1H), 8.28-8.38 (m, 1H), 10.85-11.25 (m, 1H), 13.09 (s, 1H).

LCMS(ESI): [M]⁺ m/z: calcd 555.2; found 556.2; Rt=3.424 min.

### Example 71. Compound 132 N-(6-amino-5-ethylpyridin-3-yl)-2-((2S,5R)-5-methyl-2-(4-(4-methylpiperazin-1-yl)phenyl)-4-(1-(trifluoromethyl)cyclopropanecarbonyl)piperazin-1-yl)-2-oxoacetamide

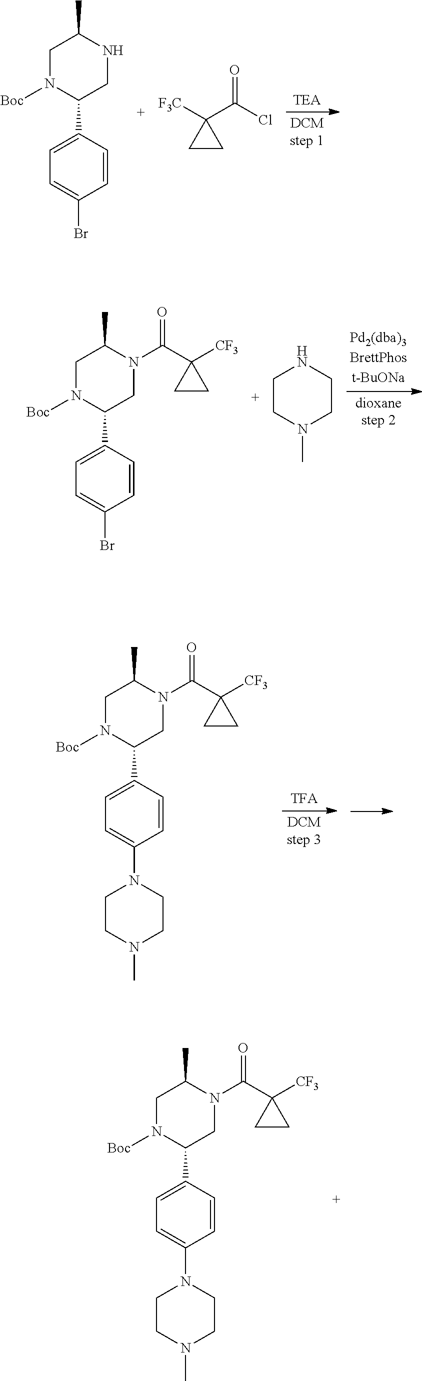

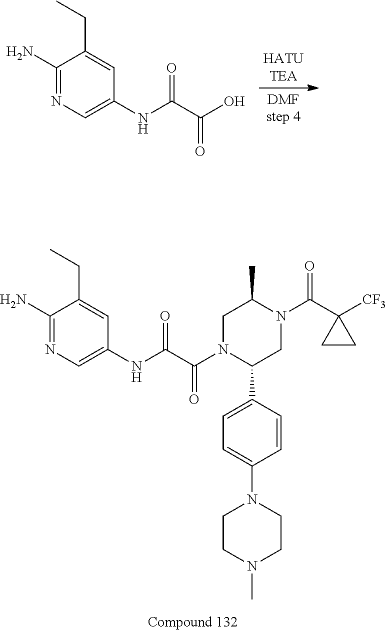 [SMILES: CCc1cc(NC(=O)C(=O)O)cnc1N ; CCc1cc(NC(=O)C(=O)N2C[C@@H](C)N(C(=O)C3(C(F)(F)F)CC3)C[C@@H]2c2ccc(N3CCN(C)CC3)cc2)cnc1N]

(2S,5R)-tert-butyl 2-(4-bromophenyl)-5-methylpiperazine-1-carboxylate is prepared by Scheme 1.4 step 6. Yield: 5.4 g of crude. LCMS(ESI): [M]⁺ m/z: calcd 355.2; found 356.2; Rt=0.989 min.

### Step 1: Synthesis of (2S,5R)-tert-butyl 2-(4-bromophenyl)-5-methyl-4-(1-(trifluoromethyl)cyclopropanecarbonyl)piperazine-1-carboxylate

tert-Butyl (2S,5R)-2-(4-bromophenyl)-5-methyl-piperazine-1-carboxylate (0.9 g, 2.53 mmol) and TEA (769.03 mg, 7.60 mmol, 1.06 mL) were mixed together in DCM (20 mL) and the resulting solution was cooled to 0° C. in an ice/MeOH bath. 1-(Trifluoromethyl)cyclopropanecarbonyl chloride (437.08 mg, 2.53 mmol) was added dropwise to the previous solution and the resulting mixture was allowed to warm to rt and stirred overnight. The reaction mixture was diluted with DCM and the resulting solution was washed with water, dried over Na₂SO₄, filtered and evaporated to obtain tert-butyl (2S,5R)-2-(4-bromophenyl)-5-methyl-4-[1-(trifluoromethyl)cyclopropanecarbonyl]piperazine-1-carboxylate (1.1 g, 2.24 mmol, 88.37% yield) which was used in the next step without further purification.

LCMS(ESI): [M-t-Bu]⁺ m/z: calcd 435.2; found 436.2; Rt=1.637 min.

### Step 2: Synthesis of (2S,5R)-tert-butyl 5-methyl-2-(4-(4-methylpiperazin-1-yl)phenyl)-4-(1-(trifluoromethyl)cyclopropanecarbonyl)piperazine-1-carboxylate

tert-Butyl (2S,5R)-2-(4-bromophenyl)-5-methyl-4-[1-(trifluoromethyl)cyclopropanecarbonyl]piperazine-1-carboxylate (1.1 g, 2.24 mmol), 1-methylpiperazine (224.24 mg, 2.24 mmol, 248.33 μL), sodium tert-butoxide (430.29 mg, 4.48 mmol) and 2-(dicyclohexylphosphino)_{3,6}-dimethoxy-2′,4′,6′-triisopropyl-1,1′-biphenyl (120.17 mg, 223.88 mol) were mixed together in dioxane (25 mL) and the resulting mixture was evacuated and backfilled three times with argon. tris(Dibenzylideneacetone)dipalladium (O) (102.50 mg, 111.94 mol) was added to the previous mixture and the resulting mixture was heated at 100° C. (oil bath) overnight. The reaction mixture was cooled and filtered, the filtrate was concentrated in vacuum to give crude product which was purified by FCC (MeOH in MTBE from 0% to 100%) to give tert-butyl (2S,5R)-5-methyl-2-[4-(4-methylpiperazin-1-yl)phenyl]-4-[1-(trifluoromethyl)cyclopropanecarbonyl]piperazine-1-carboxylate (0.8 g, 1.57 mmol, 69.99% yield).

LCMS(ESI): [M]⁺ m/z: calcd 510.2; found 511.2; Rt=1.057 min.

### Step 3: Synthesis of ((2R,5S)-2-methyl-5-(4-(4-methylpiperazin-1-yl)phenyl)piperazin-1-yl)(1-(trifluoromethyl)cyclopropyl)methanone

tert-Butyl (2,5SR)-5-methyl-2-[4-(4-methylpiperazin-1-yl)phenyl]-4-[1-(trifluoromethyl) cyclopropanecarbonyl]piperazine-1-carboxylate (0.8 g, 1.57 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain [(2R,5S)-2-methyl-5-[4-(4-methylpiperazin-1-yl)phenyl]piperazin-1-yl]-[1-(trifluoromethyl)cyclopropyl]methanone (0.36 g, 877.03 mol, 55.98% yield) which was used in the next step without further purification.

LCMS(ESI): [M]⁺ m/z: calcd 410.2; found 411.2; Rt=0.308 min.

### Step 4: Synthesis of N-(6-amino-5-ethylpyridin-3-yl)-2-((2S,5R)-5-methyl-2-(4-(4-methylpiperazin-1-yl)phenyl)-4-(1-(trifluoromethyl)cyclopropanecarbonyl)piperazin-1-yl)-2-oxoacetamide (Compound 132)

Prepared by Scheme 1.1 step 10A. Yield: 50 mg (34.11%).

HPLC conditions: Column: SunFire C18 100*19 mm, 5 microM; 2-10 min 20-55% water-MeCN 30 ml/min; (loading pump 4 ml/min MeCN).

Compound 132: ¹H NMR (600 MHz, DMSO-d₆) δ (ppm) 0.92-1.00 (m, 1H), 1.11 (t, 3H), 1.14-1.80 (m, 9H), 2.16-2.21 (m, 3H), 2.39-2.42 (m, 4H), 2.68-3.20 (m, 5H), 3.60-4.89 (m, 3H), 5.15-5.73 (m, 3H), 6.77-6.94 (m, 2H), 7.03-7.18 (m, 2H), 7.46-7.58 (m, 1H), 8.01-8.12 (m, 1H), 10.42-10.69 (m, 1H). LCMS(ESI): [M]⁺ m/z: calcd 601.2; found 602.2; Rt=2.070 min.

### Example 72. Compound 24 N-(6-amino-5-cyclopropylpyridin-3-yl)-2-((2S,5R)-2-(3-chloro-4-fluorophenyl)-4-isobutyryl-5-methylpiperazin-1-yl)-2-oxoacetamide

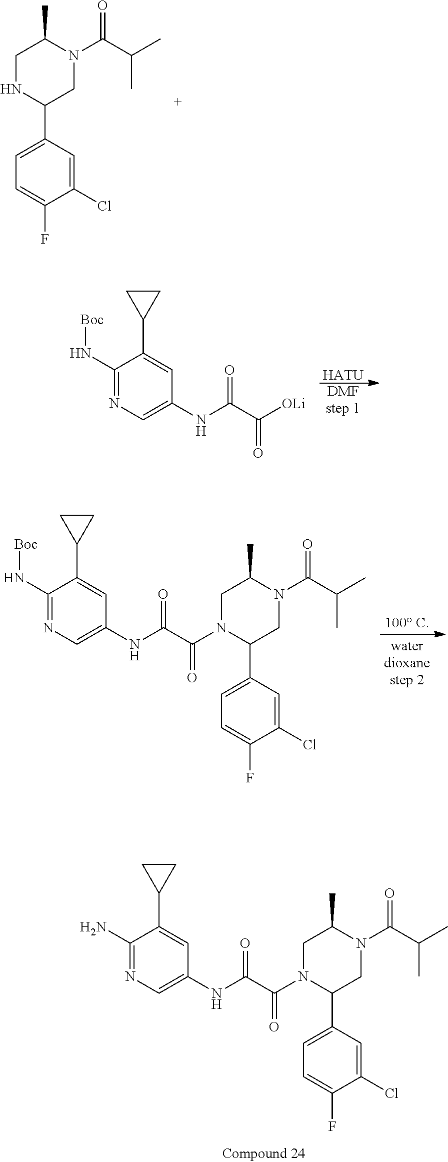

### Step 1: Synthesis of tert-butyl (5-(2-((2S,5R)-2-(3-chloro-4-fluorophenyl)-4-isobutyryl-5-methylpiperazin-1-yl)-2-oxoacetamido)-3-cyclopropylpyridin-2-yl)

The synthesis of 1-((2R)-5-(3-chloro-4-fluorophenyl)-2-methylpiperazin-1-yl)-2-methylpropan-1-one is given by tert-Butyl (2S,5R)-2-(3-chloro-4-fluoro-phenyl)-5-methyl-4-(2-methylpropanoyl)piperazine-1-carboxylate (0.27 g, 676.86 μmol) was dissolved in DCM (10 mL) and TFA (385.89 mg, 3.38 mmol, 260.74 L) was added to reaction mixture at OoC then the solution was allowed to warm to 25° C. and stirred for 16 hr. Reaction mixture was diluted with DCM (30 ml) and washed by saturated aqueous NaHCO₃ (3*30 ml). The resulting organic phase was dried over Na₂SO₄ and evaporated under reduced pressure to afford 1-[(2R,5S)-5-(3-chloro-4-fluoro-phenyl)-2-methyl-piperazin-1-yl]-2-methyl-propan-1-one (0.12 g, 401.63 μmol, 59.34% yield).

LCMS(ESI): [M]⁺ m/z: calcd 298.2; found 299.2; Rt=0.947 min.

Prepared by Scheme 1.4, step 9A. Yield: 0.706 g of crude.

LCMS(ESI): [M]⁺ m/z: calcd 602.2; found 603.2; Rt=1.484 min.

### Step 2: Synthesis of N-(6-amino-5-cyclopropylpyridin-3-yl)-2-((2S,5R)-2-(3-chloro-4-fluorophenyl)-4-isobutyryl-5-methylpiperazin-1-yl)-2-oxoacetamide (Compound 24)

Prepared by Boc Group Deprotection Method A. Yield: 45.7 mg (7.76%).

HPLC conditions: Column: YMC Triart C18 100*20 mm, 5 microM; 0-5 min 20-60% MeCN/water+0.10% NH₄OH 30 ml/min; (loading pump 4 ml/min MeCN).

Compound 24: ¹H NMR (600 MHz, dmso) 6 0.38-0.50 (m, 2H), 0.71-0.91 (m, 5H), 0.91-1.02 (m, 3H), 1.06-1.28 (m, 3H), 1.59-1.70 (m, 1H), 2.63-2.82 (m, 1H), 3.12-3.23 (m, 1H), 3.62-4.92 (m, 3H), 5.31-5.68 (m, 1H), 5.73-5.81 (m, 2H), 7.09-7.56 (m, 4H), 7.86-8.08 (m, 1H), 10.03-10.80 (m, 1H).

LCMS(ESI): [M]⁺ m/z: calcd 501.2; found 502.2; Rt=2.216 min.

### Example 73. Compound 14 N-(6-amino-5-cyclopropylpyridin-3-yl)-2-((2S,5R)-2-(3-chloro-4-fluorophenyl)-5-methyl-4-(1-(trifluoromethyl)cyclopropanecarbonyl)piperazin-1-yl)-2-oxoacetamide

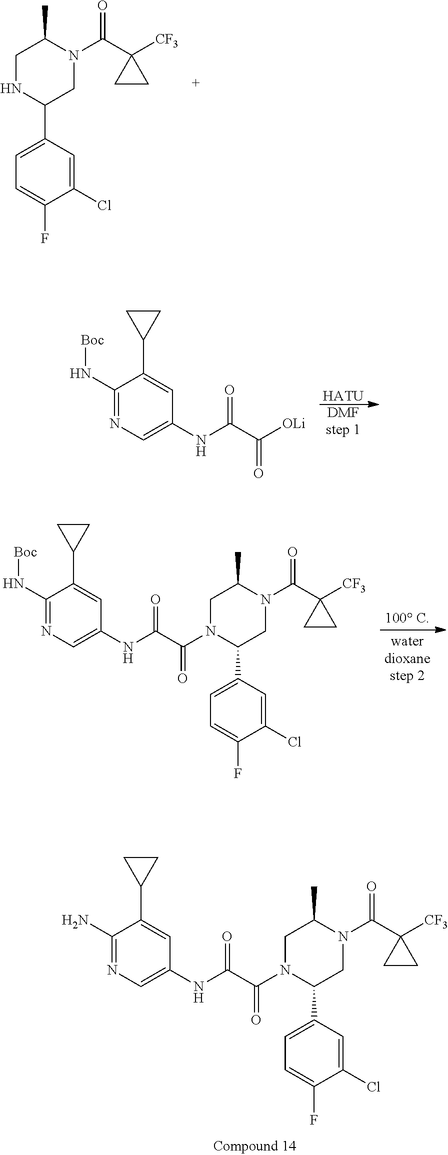

### Step 1: Synthesis of tert-butyl (5-(2-((2S,5R)-2-(3-chloro-4-fluorophenyl)-5-methyl-4-(1-(trifluoromethyl)cyclopropanecarbonyl)piperazin-1-yl)-2-oxoacetamido)-3-cyclopropylpyridin-2-yl)carbamate

The synthesis of ((2R)-5-(3-chloro-4-fluorophenyl)-2-methylpiperazin-1-yl)(1-(trifluoromethyl)cyclopropyl)methanone is given in Compound 29

Prepared by Scheme 1.4 step 9A. Yield: 0.78 g of crude.

LCMS(ESI): [M]⁺ m/z: calcd 668.2; found 669.2; Rt=1.537 min.

### Step 2: Synthesis of N-(6-amino-5-cyclopropylpyridin-3-yl)-2-((2S,5R)-2-(3-chloro-4-fluorophenyl)-5-methyl-4-(1-(trifluoromethyl)cyclopropanecarbonyl)piperazin-1-yl)-2-oxoacetamide (Compound 14)

Prepared by Boc Group Deprotection Method A. Yield: 49 mg (7.39%).

HPLC conditions: Column: YMC Triart C18 100*20 mm, 5 microM; 0-5 min 30-45% MeCN/water+0.10% NH₄OH 30 ml/min; (loading pump 4 ml/min MeCN).

Compound 14: ¹H NMR (600 MHz, dmso) 6 0.40-0.49 (m, 2H), 0.84-0.90 (m, 2H), 0.90-1.57 (m, 8H), 1.59-1.71 (m, 1H), 2.89-3.27 (m, 1H), 3.60-4.98 (m, 3H), 5.38-5.86 (m, 3H), 7.18-7.52 (m, 4H), 8.00-8.12 (m, 1H), 10.36-10.77 (m, 1H).

LCMS(ESI): [M]⁺ m/z: calcd 567.2; found 568.2; Rt=2.641 min.

### Example 74. Compound 60 N-(6-amino-5-ethylpyridin-3-yl)-2-((2S,5R)-2-(4-fluorophenyl)-5-methyl-4-(1-methylcyclopropanecarbonyl)piperazin-1-yl)-2-oxoacetamide

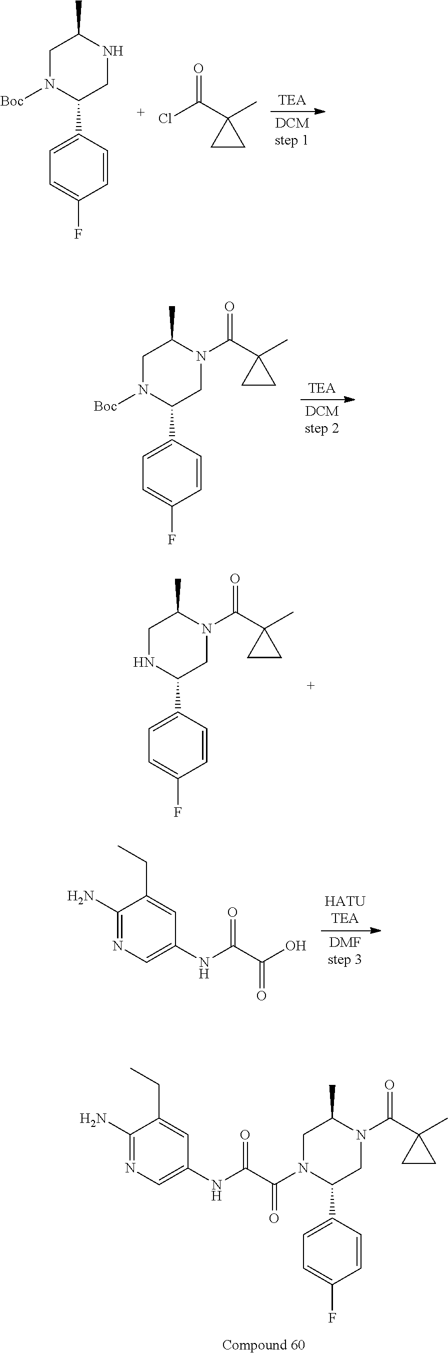

### Step 1: Synthesis of (2S,5R)-tert-butyl 2-(4-fluorophenyl)-5-methyl-4-(1-methylcyclopropanecarbonyl)piperazine-1-carboxylate

(2S,5R)-tert-butyl 2-(4-fluorophenyl)-5-methylpiperazine-1-carboxylate was prepared by Scheme 1.4, step 6. Yield: 1.3 g (27.24%).CC conditions: The crude product was purified by silica gel with MTBE/MeOH (0˜100%) as an eluent mixture.)

Prepared by Scheme 1.4 step 7A. Yield: 0.24 g (93.83%).

LCMS(ESI): [M-t-Bu]⁺ m/z: calcd 320.2; found 321.2; Rt=1.546 min.

### Step 2: Synthesis of ((2R,5S)-5-(4-fluorophenyl)-2-methylpiperazin-1-yl)(1-methylcyclopropyl)methanone

tert-Butyl (2S,5R)-2-(4-fluorophenyl)-5-methyl-4-(1-methylcyclopropanecarbonyl)piperazine-1-carboxylate (0.24 g, 637.51 mol) was dissolved in TFA (3 mL) and DCM (3 mL) was added thereto. The resulting mixture was stirred for 1 hr.

The reaction mixture was poured into aq·K₂CO₃ solution and the resulting mixture was extracted with DCM. Combined organic layers were dried over Na₂SO₄, filtered and evaporated to obtain [(2R,5S)-5-(4-fluorophenyl)-2-methyl-piperazin-1-yl]-(1-methylcyclopropyl)methanone (0.18 g, crude) which was used in the next step without further purification.

LCMS(ESI): [M]⁺ m/z: calcd 276.2; found 277.2; Rt=0.689 min.

### Step 3: Synthesis of N-(6-amino-5-ethylpyridin-3-yl)-2-((2S,5R)-2-(4-fluorophenyl)-5-methyl-4-(1-methylcyclopropanecarbonyl)piperazin-1-yl)-2-oxoacetamide (Compound 60)

Prepared by Scheme 1.4, step 9A. Yield: 26 mg (15.37%).

HPLC conditions: Column: SunFire C18 100*19 mm, 5 microM; 2-10 min 0-50% MeCN; (loading pump 4 ml/min MeCN).

Compound 60: ¹H NMR (600 MHz, DMSO-d₆) δ (ppm) 0.40-0.54 (m, 2H), 0.57-0.86 (m, 2H), 1.05 (s, 3H), 1.09-1.13 (m, 3H), 1.19-1.35 (m, 3H), 2.38-2.41 (m, 2H), 3.17 (s, 2H), 3.60-4.08 (m, 1H), 4.09-4.98 (m, 2H), 5.29-5.75 (m, 3H), 7.16-7.21 (m, 2H), 7.27-7.42 (m, 2H), 7.46-7.58 (m, 1H), 7.99-8.13 (m, 1H), 10.48-10.73 (m, 1H).

LCMS(ESI): [M]⁺ m/z: calcd 467.2; found 468.2; Rt=0.987 min.

### Example 75. Compound 105 N-(6-amino-5-ethylpyridin-3-yl)-2-((2S,5R)-5-methyl-4-(1-methylcyclopropanecarbonyl)-2-(4-(4-methylpiperazin-1-yl)phenyl)piperazin-1-yl)-2-oxoacetamide

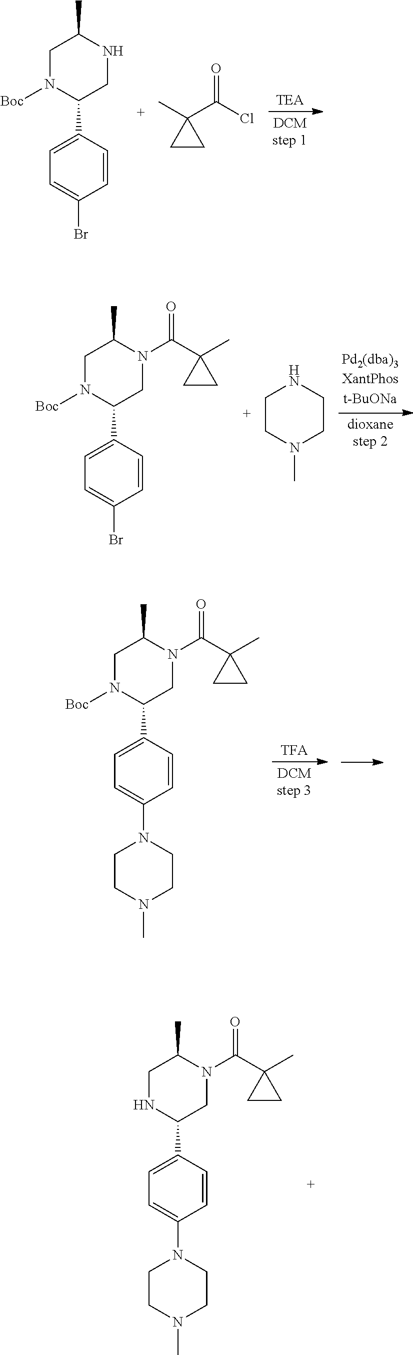

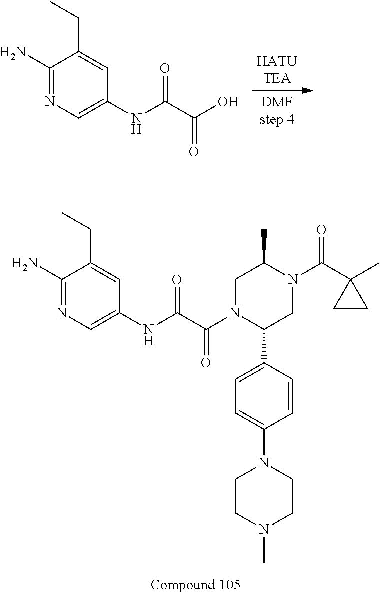 [SMILES: CCc1cc(NC(=O)C(=O)O)cnc1N ; CCc1cc(NC(=O)C(=O)N2C[C@@H](C)N(C(=O)C3(C)CC3)C[C@@H]2c2ccc(N3CCN(C)CC3)cc2)cnc1N]

(2S,5R)-tert-butyl 2-(4-bromophenyl)-5-methylpiperazine-1-carboxylate was prepared by Scheme 1.4 step 6. Yield: 5.4 g of crude.

LCMS(ESI): [M]⁺ m/z: calcd 355.2; found 356.2; Rt=0.989 min.

### Step 1: Synthesis of (2S,5R)-tert-butyl 2-(4-bromophenyl)-5-methyl-4-(1-methylcyclopropanecarbonyl)piperazine-1-carboxylate

tert-Butyl (2S,5R)-2-(4-bromophenyl)-5-methyl-piperazine-1-carboxylate (825 mg, 2.32 mmol) and TEA (704.95 mg, 6.97 mmol, 971.00 L) were mixed together in DCM (25 mL) and the resulting solution was cooled to 0° C. in an ice/MeOH bath. 1-Methylcyclopropanecarbonyl chloride (275.32 mg, 2.32 mmol) was added dropwise to the previous solution and the resulting mixture was allowed to warm to rt and stirred overnight. The reaction mixture was diluted with DCM and the resulting solution was washed with water, dried over Na₂SO₄, filtered and evaporated to obtain tert-butyl (2S,5R)-2-(4-bromophenyl)-5-methyl-4-(1-methylcyclopropanecarbonyl)piperazine-1-carboxylate (830 mg, 1.90 mmol, 81.72% yield) which was used in the next step without further purification.

LCMS(ESI): [M]⁺ m/z: calcd 437.2; found 438.2; Rt=1.478 min.

### Step 2: Synthesis of (2S,5R)-tert-butyl 5-methyl-4-(1-methylcyclopropanecarbonyl)-2-(4-(4-methylpiperazin-1-yl)phenyl)piperazine-1-carboxylate

tert-Butyl (2S,5R)-2-(4-bromophenyl)-5-methyl-4-(1-methylcyclopropanecarbonyl)piperazine-1-carboxylate (830 mg, 1.90 mmol), 1-methylpiperazine (190.08 mg, 1.90 mmol, 210.50 μL), 4,5-bis(diphenylphosphino)-9,9-dimethylxanthene (109.81 mg, 189.77 μmol) and sodium tert-butoxide (273.55 mg, 2.85 mmol) were mixed together in dioxane (25 mL) and the resulting mixture was evacuated and backfilled three times with argon. tris(Dibenzylideneacetone)dipalladium (O) (86.89 mg, 94.89 mol) was added to the previous mixture and the resulting mixture was heated at 100° C. (oil bath) overnight. The reaction mixture was cooled and diluted with water (50 ml). The resulting mixture was extracted with EtOAc (3*30 ml). Combined organic layers were washed with brine (2*20 ml), dried over Na₂SO₄, filtered and evaporated to obtain tert-butyl (2S,5R)-5-methyl-4-(1-methylcyclopropanecarbonyl)-2-[4-(4-methylpiperazin-1-yl)phenyl]piperazine-1-carboxylate (788 mg, crude).

LCMS(ESI): [M]⁺ m/z: calcd 456.2; found 457.2; Rt=1.072 min.

### Step 3: Synthesis of ((2R,5S)-2-methyl-5-(4-(4-methylpiperazin-1-yl)phenyl)piperazin-1-yl)(1-methylcyclopropyl)methanone

tert-Butyl (2S,5R)-5-methyl-4-(1-methylcyclopropanecarbonyl)-2-[4-(4-methylpiperazin-1-yl)phenyl]piperazine-1-carboxylate (788 mg, 1.73 mmol) was dissolved in DCM (5 mL) and TFA (5 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated. The obtained (1-methylcyclopropyl)-[(2R,5S)-2-methyl-5-[4-(4-methylpiperazin-1-yl)phenyl]piperazin-1-yl]methanone (543 mg, 1.52 mmol, 88.26% yield) was used in the next step without further purification.

LCMS(ESI): [M]⁺ m/z: calcd 356.2; found 357.2; Rt=0.387 min.

### Step 4: Synthesis of N-(6-amino-5-ethylpyridin-3-yl)-2-((2S,5R)-5-methyl-4-(1-methylcyclopropanecarbonyl)-2-(4-(4-methylpiperazin-1-yl)phenyl)piperazin-1-yl)-2-oxoacetamide (Compound 105)

Prepared by Scheme 1.1 step 10A. Yield: 35.8 mg (8.96%).

HPLC conditions:

1-st Run: Column: SunFire C18 100*19 mm, 5 microM; 2-10 min 30-60% MeOH+NH₃ 30 ml/min; (loading pump 4 ml/min MeOH).

2-nd Run: Column: SunFire C18 100*19 mm, 5 microM; 2-10 min 80-75% MeCN+FA 30 ml/min; (loading pump 4 ml/min MeCN).

Compound 105: ¹H NMR (600 MHz, DMSO-d₆) δ (ppm) 0.38-0.55 (m, 2H), 0.55-0.88 (m, 2H), 1.04-1.09 (m, 3H), 1.09-1.12 (m, 3H), 1.12-1.37 (m, 3H), 2.18-2.24 (m, 3H), 2.33-2.39 (m, 2H), 2.41-2.45 (m, 4H), 3.09-3.13 (m, 4H), 3.61-3.67 (m, 2H), 3.95-4.11 (m, 1H), 4.16-4.64 (m, 2H), 4.71-5.25 (m, 1H), 5.59-5.69 (m, 2H), 6.83-6.91 (m, 2H), 7.02-7.21 (m, 2H), 7.41-7.63 (m, 1H), 8.04-8.13 (m, 1H), 10.49-10.73 (m, 1H).

LCMS(ESI): [M]⁺ m/z: calcd 547.2; found 548.2; Rt=1.803 min.

### Example 76. Compound 8 N-(6-amino-5-cyclopropylpyridin-3-yl)-2-((2S,5R)-2-(4-fluorophenyl)-5-methyl-4-(1-methylcyclopropanecarbonyl)piperazin-1-yl)-2-oxoacetamide

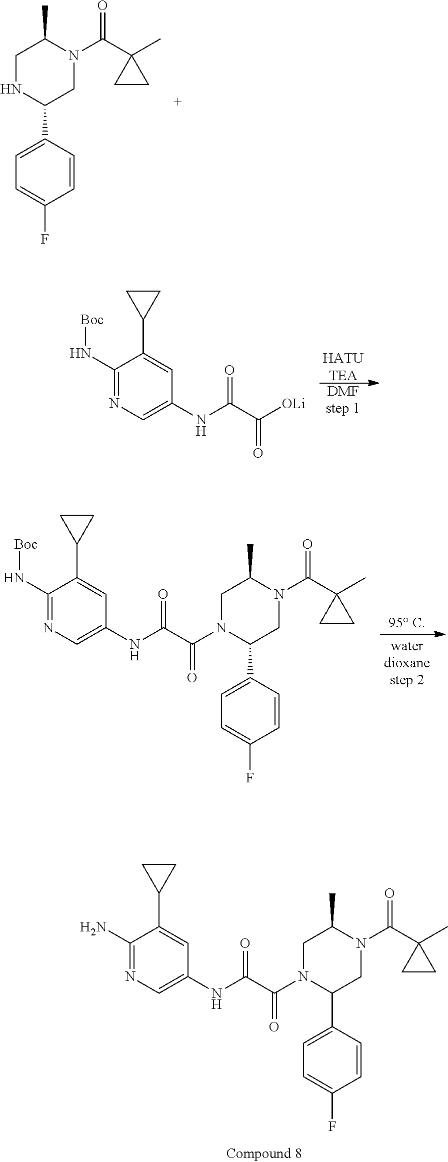

### Step 1: Synthesis of tert-butyl (3-cyclopropyl-5-(2-((2S,5R)-2-(4-fluorophenyl)-5-methyl-4-(1-methylcyclopropanecarbonyl)piperazin-1-yl)-2-oxoacetamido)pyridin-2-yl)carbamate

The synthesis of ((2R,5S)-5-(4-fluorophenyl)-2-methylpiperazin-1-yl)(1-methylcyclopropyl) methanone is give by the following procedure.

Palladium, 10% on carbon (350 mg, 328.89 umol, 10% purity) was added to the solution of [(2R,5S)-4-benzyl-5-(4-fluorophenyl)-2-methyl-piperazin-1-yl]-(1-methylcyclopropyl)methanone (1.35 g, 3.68 mmol) in MeOH (30 mL) and acetic acid (10 mL). Reaction flask was evacuated and backfilled with hydrogen (111.39 mg, 55.26 mmol) from attached balloon. Resulting mixture was stirred at 50° C. for 16 hr. Then, catalyst was filtered off and filtrate was concentrated under reduced pressure. Residue was partitioned between 10% aq. K₂CO₃ solution (20 ml) and DCM (40 ml). Organic layer was separated, dried over K₂CO₃ and concentrated in vacuum, affording [(2R,5S)-5-(4-fluorophenyl)-2-methyl-piperazin-1-yl]-(1-methylcyclopropyl)methanone (0.96 g, 3.47 mmol, 94.30% yield).

LCMS(ESI): [M]⁺ m/z: calcd 276.2; found 277.2; Rt=0.702 min.

Prepared by Scheme 1.4, step 9A. Yield: 740 mg of crude.

LCMS(ESI): [M]⁺ m/z: calcd 579.2; found 580.2; Rt=1.405 min.

### Step 2: Synthesis of N-(6-amino-5-cyclopropylpyridin-3-yl)-2-((2S,5R)-2-(4-fluorophenyl)-5-methyl-4-(1-methylcyclopropanecarbonyl)piperazin-1-yl)-2-oxoacetamide (Compound 8)

Water (1.00 g, 55.49 mmol, 1 mL) and acetic acid (105.00 mg, 1.75 mmol, 0.1 mL) were added to the solution of tert-butyl N-[3-cyclopropyl-5-[[2-[(2S,5R)-2-(4-fluorophenyl)-5-methyl-4-(1-methylcyclopropanecarbonyl)piperazin-1-yl]-2-oxo-acetyl]amino]-2-pyridyl]carbamate (740 mg, 395.75 umol) in dioxane (3 mL). Resulting mixture was stirred at 95° C. for 15 hr. Then, it was subjected to HPLC (10-35% 0-1-5 min H₂O/MeCN/0.1% FA, flow: 30 ml/min; column: Chromatorex 18 SMB100-5T 100×19 mm 5 um), affording N-(6-amino-5-cyclopropyl-3-pyridyl)-2-[(2S,5R)-2-(4-fluorophenyl)-5-methyl-4-(1-methylcyclopropanecarbonyl)piperazin-1-yl]-2-oxo-acetamide (100 mg, 208.53 umol, 52.69% yield).

Compound 8: ¹H NMR (600 MHz, DMSO-d₆) δ (ppm) 0.51 (m, 6H), 0.87 (m, 3H), 1.05 (m, 3H), 1.26 (s, 3H), 1.66 (m, 1H), 3.87 (m, 2H), 4.62 (m, 2H), 5.77 (m, 3H), 7.19 (m, 2H), 7.29 (m, 1H), 7.34 (m, 2H), 8.12 (m, 1H), 10.59 (m, 1H).

LCMS(ESI): [M]⁺ m/z: calcd 479.2; found 480.2; Rt=2.327 min.

### Example 77. Compound 89 N-(6-amino-5-ethylpyridin-3-yl)-2-((2S,5R)-4-isobutyryl-5-methyl-2-(4-(4-methylpiperazin-1-yl)phenyl)piperazin-1-yl)-2-oxoacetamide

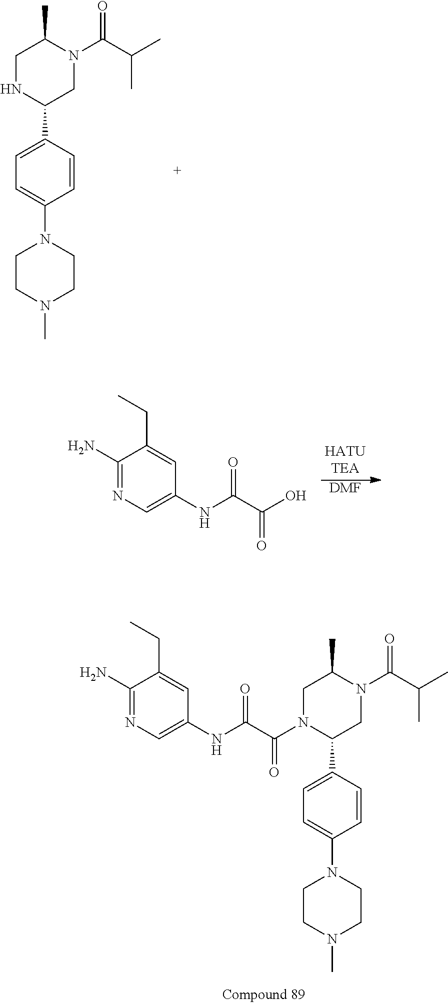

The 2-methyl-1-((2R,5S)-2-methyl-5-(4-(4-methylpiperazin-1-yl)phenyl)piperazin-1-yl)propan-1-one is by the following procedure. tert-Butyl (2S,5R)-5-methyl-2-[4-(4-methylpiperazin-1-yl)phenyl]-4-(2-methylpropanoyl)piperazine-1-carboxylate (0.6 g, 1.35 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain 2-methyl-1-[(2R,5S)-2-methyl-5-[4-(4-methylpiperazin-1-yl)phenyl]piperazin-1-yl]propan-1-one (0.2 g, 580.56 mol, 43.02% yield) which was used in the next step without further purification.

LCMS(ESI): [M]⁺ m/z: calcd 344.2; found 345.2; Rt=0.171 min.

Prepared by Scheme 1.1 step 10A. Yield: 33 mg (32.65%).

HPLC conditions: Column: SunFire C18 100*19 mm, 5 microM; 2-10 min 45-60% MeOH+NH₃ 30 ml/min; (loading pump 4 ml/min MeOH).

Compound 89: ¹H NMR (600 MHz, DMSO-d₆) δ (ppm) 0.83 (dd, 1H), 0.90 (t, 2H), 0.93 (t, 1H), 0.98-1.01 (m, 2H), 1.07-1.13 (m, 4H), 1.23 (dd, 2H), 2.17-2.21 (m, 3H), 2.34-2.38 (m, 1H), 2.40-2.43 (m, 4H), 2.71-3.20 (m, 7H), 3.42-4.92 (m, 4H), 5.18-5.67 (m, 3H), 6.84-6.94 (m, 2H), 7.06-7.20 (m, 2H), 7.30-7.56 (m, 1H), 7.90-8.09 (m, 1H), 10.35-10.62 (m, 1H).

LCMS(ESI): [M]⁺ m/z: calcd 535.2; found 536.2; Rt=1.822 min.

### Example 78. Compound 116 N-(6-amino-5-ethylpyridin-3-yl)-2-((2S,5R)-2-(4-fluorophenyl)-5-methyl-4-(1-(trifluoromethyl)cyclopropanecarbonyl)piperazin-1-yl)-2-oxoacetamide

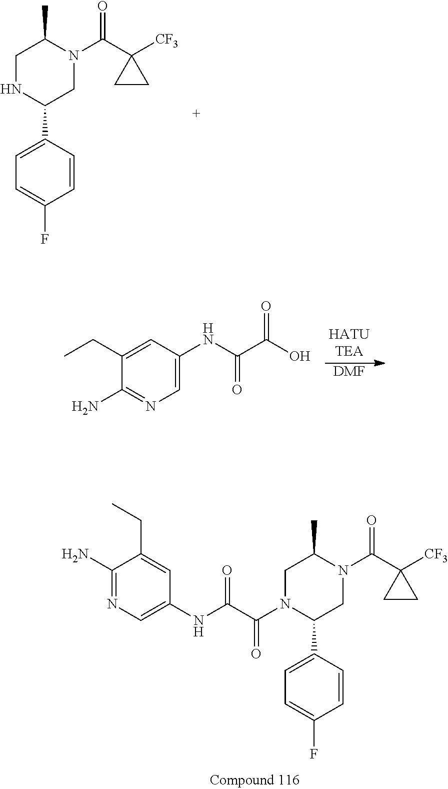

((2R,5S)-5-(4-fluorophenyl)-2-methylpiperazin-1-yl)(1-(trifluoromethyl)cyclopropyl)methanone is prepared by Scheme 1.4 step 8. Yield: 2.5 g (TFA- PGP491,c3 salt).

LCMS(ESI): [M]⁺ m/z: calcd 330.2; found 331.2; Rt=0.962 min.

Prepared by Scheme 1.4 step 9A. Yield: 9.4 mg (3.08%).

HPLC conditions: Column: SunFire C18 100*19 mm, 5 microM; 2-10 min 30-50% MeCN; (loading pump 4 ml/min MeCN).

Compound 116: ¹H NMR (600 MHz, dmso) 6 0.93-1.00 (m, 1H), 1.09-1.13 (m, 3H), 1.17-1.31 (m, 6H), 2.38-2.41 (m, 2H), 2.81-3.26 (m, 2H), 3.63-4.44 (m, 2H), 4.44-4.97 (m, 1H), 5.35-5.64 (m, 1H), 5.65-5.79 (m, 2H), 7.15-7.20 (m, 2H), 7.24-7.30 (m, 1H), 7.33-7.40 (m, 1H), 7.44-7.57 (m, 1H), 8.03-8.11 (m, 1H), 10.34-10.74 (m, 1H).

LCMS(ESI): [M]⁺ m/z: calcd 521.2; found 522.2; Rt=0.941 min.

### Example 79. Compound 33 N-(6-amino-5-ethylpyridin-3-yl)-2-((2S,5R)-2-(4-fluorophenyl)-5-methyl-4-pivaloylpiperazin-1-yl)-2-oxoacetamide

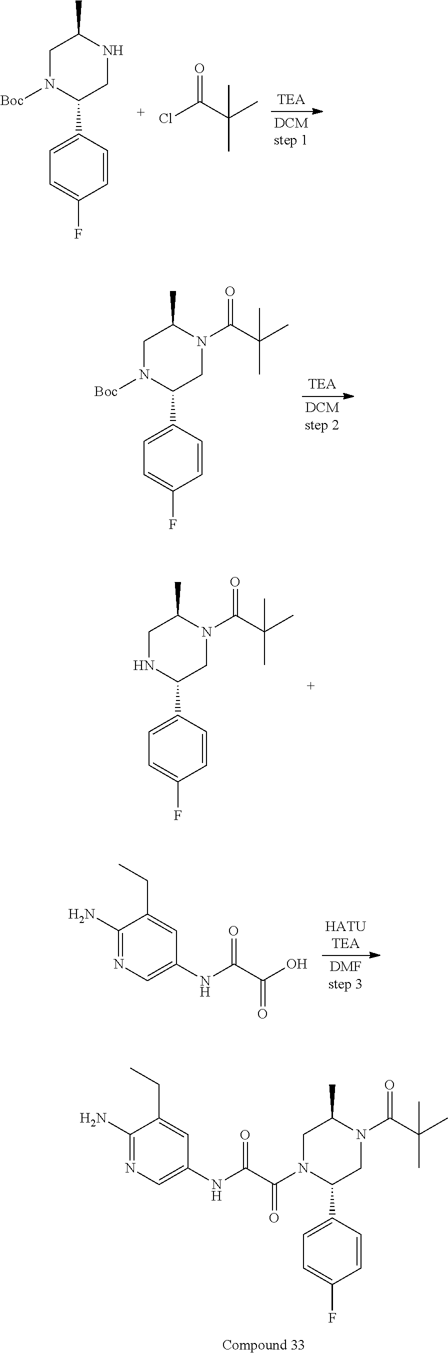

### Step 1: Synthesis of (2S,5R)-tert-butyl 2-(4-fluorophenyl)-5-methyl-4-pivaloylpiperazine-1-carboxylate

(2S,5R)-tert-butyl 2-(4-fluorophenyl)-5-methylpiperazine-1-carboxylate is prepared by Scheme 1.4 step 6. Yield: 1.3 g (27.24%). CC conditions: The crude product was purified by silica gel with MTBE/MeOH (0˜100%) as an eluent mixture.

LCMS(ESI): [M]⁺ m/z: calcd 294.2; found 295.2; Rt=1.024 min.

Prepared by Scheme 1.4 step 7A. Yield: 0.24 g (93.33%).

LCMS(ESI): [M-t-Bu]⁺ m/z: calcd 322.2; found 323.2; Rt=1.546 min.

### Step 2: Synthesis of 1-((2R,5S)-5-(4-fluorophenyl)-2-methylpiperazin-1-yl)-2,2-dimethylpropan-1-one

tert-Butyl (2S,5R)-4-(2,2-dimethylpropanoyl)-2-(4-fluorophenyl)-5-methyl-piperazine-1-carboxylate (0.24 g, 634.12 mol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain 1-[(2R,5S)-5-(4-fluorophenyl)-2-methyl-piperazin-1-yl]-2,2-dimethyl-propan-1-one (0.17 g, 610.71 μmol, 96.310% yield) which was used in the next step without further purification.

LCMS(ESI): [M]⁺ m/z: calcd 278.2; found 279.2; Rt=0.749 min.

### Step 3: Synthesis of N-(6-amino-5-ethylpyridin-3-yl)-2-((2S,5R)-2-(4-fluorophenyl)-5-methyl-4-pivaloylpiperazin-1-yl)-2-oxoacetamide (Compound 33)

Prepared by Scheme 1.4 step 9A. Yield: 13 mg (7.71%).

HPLC conditions: Column: SunFire C18 100*19 mm, 5 microM; 2-10 min 30-45% water-MeOH+NH₃; (loading pump 4 ml/min MeOH).

Compound 33: ¹H NMR (600 MHz, DMSO-d₆) δ (ppm) 1.07 (s, 9H), 1.09-1.13 (m, 3H), 1.24 (d, 3H), 2.38-2.40 (m, 2H), 3.03-3.21 (m, 2H), 3.62-4.06 (m, 1H), 4.42-4.55 (m, 1H), 4.60-5.09 (m, 1H), 5.27-5.64 (m, 1H), 5.64-5.69 (m, 2H), 7.15-7.21 (m, 2H), 7.27-7.31 (m, 1H), 7.34-7.41 (m, 1H), 7.44-7.52 (m, 1H), 7.98-8.09 (m, 1H), 10.60 (s, 1H).

LCMS(ESI): [M]⁺ m/z: calcd 469.2; found 470.2; Rt=1.071 min.

### Example 80. Compound 133 N-(6-amino-5-cyclopropylpyridin-3-yl)-2-((2S,5R)-2-(3-chlorophenyl)-4-isobutyryl-5-methylpiperazin-1-yl)-2-oxoacetamide

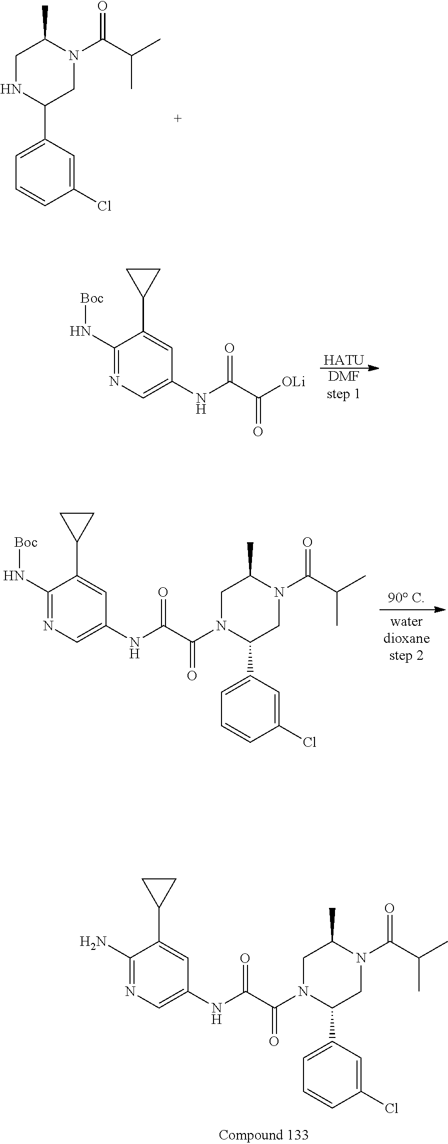

### Step 1: Synthesis of tert-butyl (5-(2-((2S,5R)-2-(3-chlorophenyl)-4-isobutyryl-5-methylpiperazin-1-yl)-2-oxoacetamido)-3-cyclopropylpyridin-2-yl)carbamate

The synthesis of 1-((2R)-5-(3-chlorophenyl)-2-methylpiperazin-1-yl)-2-methylpropan-1-one is by the following procedure. tert-Butyl (2S,5R)-2-(3-chlorophenyl)-5-methyl-4-(2-methylpropanoyl)piperazine-1-carboxylate (0.6 g, 1.58 mmol) was dissolved in CHCl₃ (15 mL) and TFA (3.70 g, 32.45 mmol, 2.5 mL) was added thereto. The resulting mixture was stirred at 25° C. for 16 hr. After completion the reaction mixture was evaporated, the crude product was poured into aq·K₂CO₃ solution (4 g in 10 ml of water) and the resulting mixture was extracted with CHCl₃ (2*10 ml). Combined organic layers were dried over Na₂SO₄, filtered and evaporated. The reaction was successful. The desired product 1-[(2R,5S)-5-(3-chlorophenyl)-2-methyl-piperazin-1-yl]-2-methyl-propan-1-one (0.44 g, 1.57 mmol, 99.48% yield) was isolated.

LCMS(ESI): [M]⁺ m/z: calcd 280.2; found 281.2; Rt=0.698 min.

Prepared by Scheme 1.4 step 9A. Yield: 0.19 g of crude.

LCMS(ESI): [M]⁺ m/z: calcd 584.2; found 585.2; Rt=1.462 min.

### Step 2: Synthesis of N-(6-amino-5-cyclopropylpyridin-3-yl)-2-((2S,5R)-2-(3-chlorophenyl)-4-isobutyryl-5-methylpiperazin-1-yl)-2-oxoacetamide (Compound 133)

Prepared by Bon Group Deprotection Method A. Yield: 60 mg (38.61%).

HPLC conditions: Column: YMC Triart C18 100*20 mm, 5 microM; 0-5 min 35-85% MeOH/water+0.1% NH₄OH 30 ml/min; (loading pump 4 ml/min MeOH).

Compound 133: ¹H NMR (600 MHz, DMSO-d₆) δ (ppm) 0.45 (m, 2H), 0.82 (m, 5H), 0.96 (m, 3H), 1.18 (m, 3H), 1.64 (m, 1H), 2.74 (m, 1H), 3.18 (m, 1H), 3.63 (m, 1H), 3.84 (m, 1H), 4.23 (m, 1H), 4.79 (m, 1H), 5.66 (m, 3H), 7.34 (m, 5H), 8.01 (m, 1H), 10.56 (s, 1H).

LCMS(ESI): [M]⁺ m/z: calcd 483.2; found 484.2; Rt=2.783 min.

### Example 81. Compound 142 2-((2S,5R)-5-methyl-2-(4-(4-methylpiperazin-1-yl)phenyl)-4-pivaloylpiperazin-1-yl)-2-oxo-N-(1H-pyrazolo[4,3-c]pyridin-7-yl)acetamide

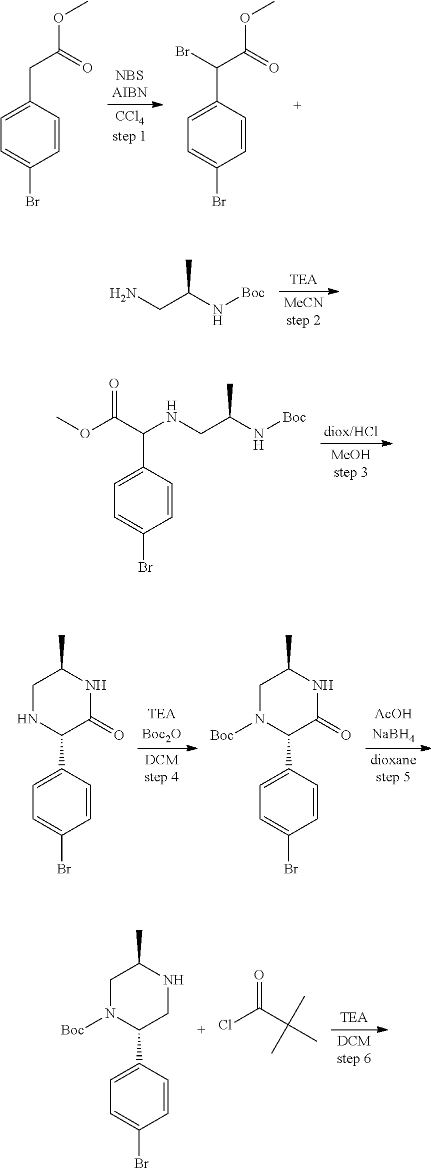

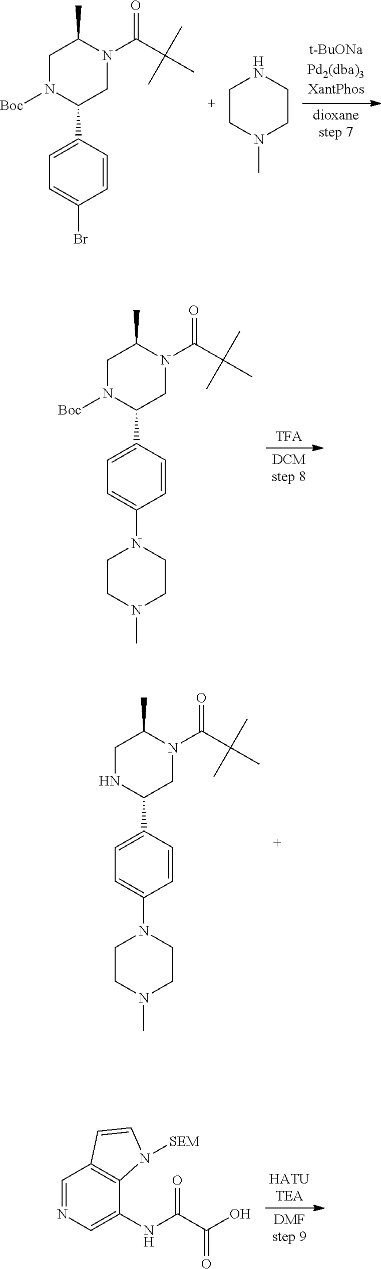

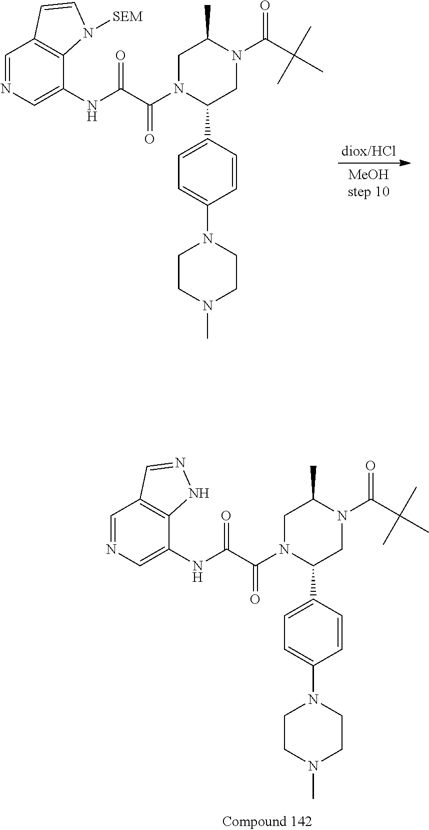 [SMILES: C[C@@H]1CN(C(=O)C(=O)Nc2cncc3ccn(COCC[Si](C)(C)C)c23)[C@@H](c2ccc(N3CCN(C)CC3)cc2)CN1C(=O)C(C)(C)C ; C[C@@H]1CN(C(=O)C(=O)Nc2cncc3cn[nH]c23)[C@@H](c2ccc(N3CCN(C)CC3)cc2)CN1C(=O)C(C)(C)C]

### Step 1: Synthesis of methyl 2-bromo-2-(4-bromophenyl)acetate

To a mixture of methyl 2-(4-bromophenyl)acetate (59.85 g, 261.27 mmol) in CCl4 (600 mL), 2,2′-azobis(2-methylpropionitrile) (4.29 g, 26.13 mmol) and N-bromosuccinimide (69.75 g, 391.91 mmol, 33.25 mL) was added in one portion. The resulting mixture was left to stir at 60° C. for 16 hr. Upon completion of the reaction, solvent was evaporated; 300 ml of water was added to residue. Water phase was extracted with MTBE (2*300 ml)m combined organic layers was washed with K₂CO₃ water solution, dried over Na₂SO₄, filtered and concentrated under reduced pressure to obtain methyl 2-bromo-2-(4-bromophenyl)acetate (72 g, crude).

¹H NMR (400 MHz, CDCl₃) δ (ppm) 3.77 (s, 3H), 5.28 (s, 1H), 7.14 (d, 2H), 7.47 (d, 2H).

### Step 2: Synthesis of methyl 2-(4-bromophenyl)-2-(((R)-2-((tert-butoxycarbonyl)amino)propyl)amino)acetate

Prepared by Scheme 1.4 step 3. Yield: 21 g of crude.

LCMS(ESI): [M]⁺ m/z: calcd 401.2; found 402.2; Rt=1.183 min.

### Step 3: Synthesis of (3S,6R)-3-(4-bromophenyl)-6-methylpiperazin-2-one

Prepared by Scheme 1.4 step 4. Yield: 7.36 g (30.82%).

LCMS(ESI): [M]⁺ m/z: calcd 269.2; found 270.2; Rt=0.216 min.

### Step 4: Synthesis of (2S,5R)-tert-butyl 2-(4-bromophenyl)-5-methyl-3-oxopiperazine-1-carboxylate

Prepared by Scheme 1.4 step 5. Yield: 10.9 g of crude.

LCMS(ESI): [M-t-Bu]⁺ m/z: calcd 313.2; found 314.2; Rt=1.199 min.

### Step 5: Synthesis of (2S,5R)-tert-butyl 2-(4-bromophenyl)-5-methylpiperazine-1-carboxylate

Prepared by Scheme 1.4 step 6. Yield: 5.4 g of crude.

LCMS(ESI): [M]⁺ m/z: calcd 355.2; found 356.2; Rt=0.989 min.

### Step 6: Synthesis of (2S,5R)-tert-butyl 2-(4-bromophenyl)-5-methyl-4-pivaloylpiperazine-1-carboxylate

Prepared by Scheme 1.4 step 7A. Yield: 1.38 g of crude.

LCMS(ESI): [M-t-Bu]⁺ m/z: calcd 383.2; found 384.2; Rt=1.490 min.

### Step 7: Synthesis of (2S,5R)-tert-butyl 5-methyl-2-(4-(4-methylpiperazin-1-yl)phenyl)-4-pivaloylpiperazine-1-carboxylate (

tert-Butyl (2S,5R)-2-(4-bromophenyl)-4-(2,2-dimethylpropanoyl)-5-methyl-piperazine-1-carboxylate (1.06 g, 2.41 mmol), 1-methylpiperazine (241.64 mg, 2.41 mmol, 267.59 μL), 4,5-bis(diphenylphosphino)-9,9-dimethylxanthene (139.59 mg, 241.25 μmol) and sodium tert-butoxide (347.76 mg, 3.62 mmol) were mixed together in dioxane (25 mL) and the resulting mixture was evacuated and backfilled three times with argon. tris(Dibenzylideneacetone)dipalladium (O) (110.46 mg, 120.62 mol) was added to the previous mixture and the resulting mixture was heated at 100° C. (oil bath) overnight. The reaction mixture was cooled and diluted with water (20 ml). The resulting mixture was extracted with EtOAc (3*10 ml). Combined organic layers were washed with brine (2*10 ml), dried over Na₂SO₄, filtered and evaporated to obtain tert-butyl (2S,5R)-4-(2,2-dimethylpropanoyl)-5-methyl-2-[4-(4-methylpiperazin-1-yl)phenyl]piperazine-1-carboxylate (1 g, crude).

LCMS(ESI): [M]⁺ m/z: calcd 458.2; found 459.2; Rt=1.041 min.

### Step 8: Synthesis of 2,2-dimethyl-1-((2R,5S)-2-methyl-5-(4-(4-methylpiperazin-1-yl)phenyl)piperazin-1-yl)propan-1-one

tert-Butyl (2S,5R)-4-(2,2-dimethylpropanoyl)-5-methyl-2-[4-(4-methylpiperazin-1-yl)phenyl]piperazine-1-carboxylate (1.1 g, 2.40 mmol) was dissolved in DCM (20 mL), then TFA (7.02 g, 61.54 mmol, 4.74 mL) was added and it was stirred 16 hr at rt. The reaction mixture was poured into aq·K₂CO₃ solution and the residue was extracted with DCM (2*25 ml). Combined organic layers were fried over Na₂SO₄, filtered and concentrated in vacuum to obtain 2,2-dimethyl-1-[(2R,5S)-2-methyl-5-[4-(4-methylpiperazin-1-yl)phenyl]piperazin-1-yl]propan-1-one (230 mg, 641.53 mol, 26.75% yield).

LCMS(ESI): [M]⁺ m/z: calcd 358.2; found 359.2; Rt=0.631 min.

### Step 9: Synthesis of 2-((2S,5R)-5-methyl-2-(4-(4-methylpiperazin-1-yl)phenyl)-4-pivaloylpiperazin-1-yl)-2-oxo-N-(1-((2-(trimethylsilyl)ethoxy)methyl)-1H-pyrazolo[4,3-c]pyridin-7-yl)acetamide

Prepared by Scheme 1.4 step 9A. Yield: 87 mg (57.6%).

HPLC conditions: Column: SunFire C18 100*19 mm, 5 microM; 2-10 min 40-60% MeOH+FA; (loading pump 4 ml/min MeOH).

LCMS(ESI): [M]⁺ m/z: calcd 676.2; found 677.2; Rt=0.912 min.

### Step 10: Synthesis of 2-((2S,5R)-5-methyl-2-(4-(4-methylpiperazin-1-yl)phenyl)-4-pivaloylpiperazin-1-yl)-2-oxo-N-(1H-pyrazolo[4,3-c]pyridin-7-yl)acetamide (Compound 142)

To a solution of 2-oxo-2-[(2S,5R)-4-(2,2-dimethylpropanoyl)-5-methyl-2-[4-(4-methylpiperazin-1-yl)phenyl]piperazin-1-yl]-N-[1-(2-trimethylsilylethoxymethyl)pyrazolo[4,3-c]pyridin-7-yl]acetamide (87 mg, 128.52 μmol) in MeOH (5.00 mL) was added hydrogen chloride solution 4.0 M in dioxane (800.00 mg, 21.94 mmol, 1.00 mL) at 26° C. The resulting mixture was left to stir for 14 hr. The resulting mixture was evaporated to dryness and then submitted to reverse phase HPLC (SYSTEM 2-10 min 20-40% MeOH+NH₃ flow 30 ml/min (loading pump 4 ml/min MeOH), Column Sun Fire C18 100*19 mm) to afford 2-oxo-N-(1H-pyrazolo[4,3-c]pyridin-7-yl)-2-[(2S,5R)-4-(2,2-dimethylpropanoyl)-5-methyl-2-[4-(4-methylpiperazin-1-yl)phenyl]piperazin-1-yl]acetamide (31.1 mg, 56.89 mol, 44.27% yield).

Compound 142: ¹H NMR (600 MHz, DMSO-d₆) δ (ppm) 1.12 (s, 9H), 1.23-1.31 (m, 3H), 2.16-2.20 (m, 3H), 2.36-2.39 (m, 2H), 2.42 (t, 2H), 3.00-3.08 (m, 2H), 3.09-3.12 (m, 2H), 3.47-5.70 (m, 6H), 6.82-7.19 (m, 4H), 8.31-8.37 (m, 1H), 8.42-8.51 (m, 1H), 8.90-8.99 (m, 1H), 11.17 (s, 1H), 13.07 (s, 1H).

LCMS(ESI): [M]⁺ m/z: calcd 546.2; found 547.2; Rt=0.773 min.

### Example 82. Compound 29 N-(6-amino-5-methylpyridin-3-yl)-2-((2S,5R)-2-(3-chloro-4-fluorophenyl)-5-methyl-4-(1-(trifluoromethyl)cyclopropanecarbonyl)piperazin-1-yl)-2-oxoacetamide

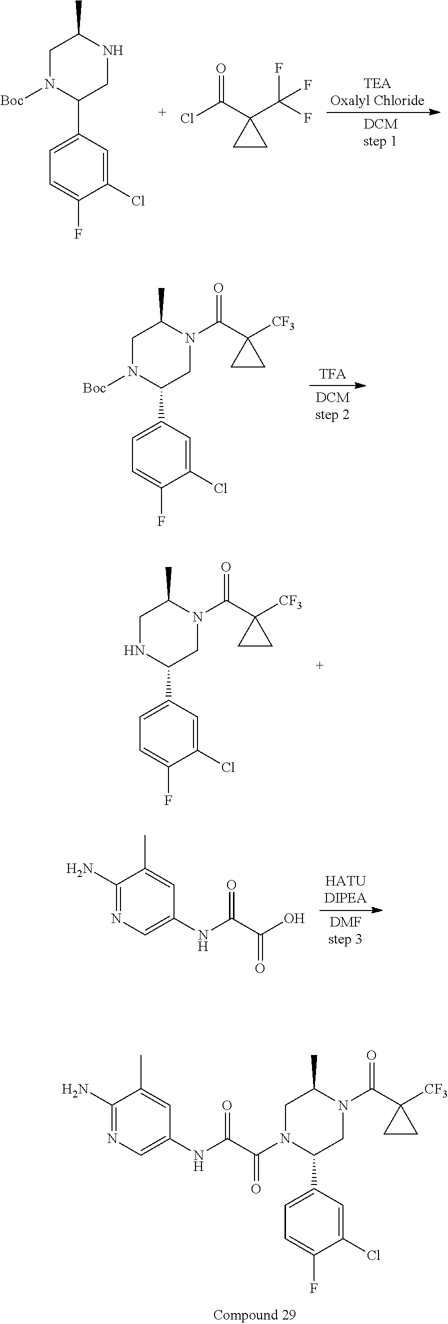

### Step 1: Synthesis of (2S,5R)-tert-butyl 2-(3-chloro-4-fluorophenyl)-5-methyl-4-(1-(trifluoromethyl)cyclopropanecarbonyl)piperazine-1-carboxylate

(5R)-tert-butyl 2-(3-chloro-4-fluorophenyl)-5-methylpiperazine-1-carboxylate is prepared by Scheme 1.4 step 6. Yield: 1.4 g of crude.

LCMS(ESI): [M-Boc]⁺ m/z: calcd 228.2; found 229.2; Rt=0.933 min

Prepared by Scheme 1.4 step, 7B Yield: 0.29 g (82.05%).

LCMS(ESI): [M-t-Bu]⁺ m/z: calcd 408.2; found 409.2; Rt=1.640 min.

### Step 2: Synthesis of ((2R,5S)-5-(3-chloro-4-fluorophenyl)-2-methylpiperazin-1-yl)(1-(trifluoromethyl)cyclopropyl)methanone

tert-Butyl (2S,5R)-2-(3-chloro-4-fluoro-phenyl)-5-methyl-4-[1-(trifluoromethyl)cyclopropane carbonyl]piperazine-1-carboxylate (0.29 g, 623.82 μmol) was dissolved in DCM (30.23 mL) and TFA (355.65 mg, 3.12 mmol, 240.30 L) was added to reaction mixture at 0° C. then the solution was allowed to warm to 25° C. and stirred at 25° C. for 16 hr. Reaction mixture was diluted with DCM (30 ml.) and washed by saturated aqueous NaHCO₃ (3*30 ml). The resulting organic phase was dried over Na₂SO₄ and evaporated under reduced pressure to afford [(2R,5S)-5-(3-chloro-4-fluoro-phenyl)-2-methyl-piperazin-1-yl]-[1-(trifluoromethyl)cyclopropyl]methanone (0.18 g, 493.47 mol, 79.10% yield).

LCMS(ESI): [M]⁺ m/z: calcd 364.2; found 365.2; Rt=1.044 min.

### Step 3: Synthesis of N-(6-amino-5-methylpyridin-3-yl)-2-((2S,5R)-2-(3-chloro-4-fluorophenyl)-5-methyl-4-(1-(trifluoromethyl)cyclopropanecarbonyl)piperazin-1-yl)-2-oxoacetamide

Prepared by Scheme 1.4 step 9B. Yield: 51 mg (19.07%).

HPLC conditions: Column: Chromarex C18 100*19 mm, 5 microM; 0-5 min 20-40% MeCN/water+0.2% FA 30 ml/min; (loading pump 4 ml/min MeCN).

Compound 29: ¹H NMR (DMSO-d₆, 600 MHz): δ (ppm) 1.02-1.27 (m, 6H), 2.01-2.03 (m, 3H), 3.03 (m, 1H), 3.66-4.49 (m, 4H), 4.91 (m, 1H), 5.38-5.75 (m, 3H), 7.21-7.54 (m, 4H), 8.01-8.12 (m, 1H), 10.62-10.70 (m, 1H).

LCMS(ESI): [M]⁺ m/z: calcd 541.2; found 542.2; Rt=3.067 min.

### Example 83. Compound 58 N-(6-amino-5-ethylpyridin-3-yl)-2-((2S,5R)-5-methyl-4-(1-methylcyclopropanecarbonyl)-2-phenylpiperazin-1-yl)-2-oxoacetamide

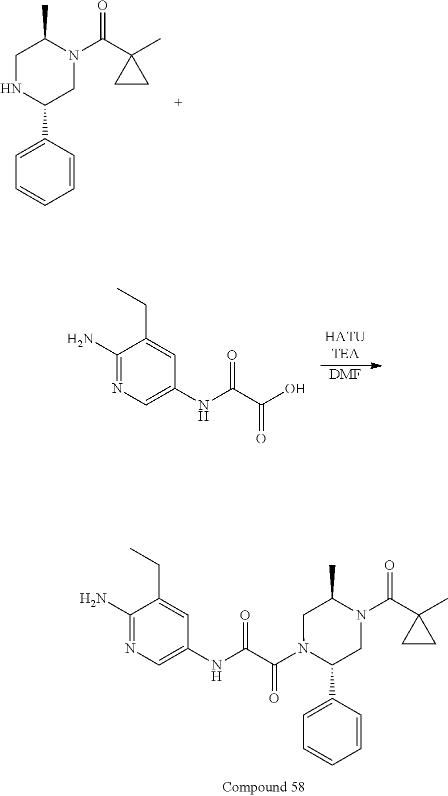

((2R,5S)-2-methyl-5-phenylpiperazin-1-yl)(1-methylcyclopropyl)methanone is prepared by Scheme 1.1 step 9. Yield: 0.2 g (79.29%).

LCMS(ESI): [M]⁺ m/z: calcd 258.2; found 259.2; Rt=0.692 min.

Prepared by Scheme 1.1 step 10A. Yield: 38.4 mg (20.06%).

HPLC conditions: Column: YMC Triart C18 100*20 mm, 5 microM; 0-1-6 min 25-25-60% water-MeCN+0.1% NH₄OH 30 ml/min; (loading pump 4 ml/min MeOH).

Compound 58: ¹H NMR (600 MHz, DMSO-d₆) δ (ppm) 0.41-0.82 (m, 4H), 1.04 (s, 3H), 1.09-1.13 (m, 3H), 1.20-1.38 (m, 3H), 2.40 (q, 2H), 2.67-3.22 (m, 1H), 3.67-5.37 (m, 4H), 5.49-6.01 (m, 3H), 7.24-7.29 (m, 2H), 7.32-7.37 (m, 3H), 7.46-7.58 (m, 1H), 8.02-8.13 (m, 1H), 10.46-10.75 (m, 1H).

LCMS(ESI): [M]⁺ m/z: calcd 449.2; found 450.2; Rt=1.987 min.

### Example 84. Compound 59 N-(6-amino-5-cyclopropylpyridin-3-yl)-2-((2S,5R)-2-(3,4-difluorophenyl)-5-methyl-4-(1-methylcyclopropanecarbonyl)piperazin-1-yl)-2-oxoacetamide

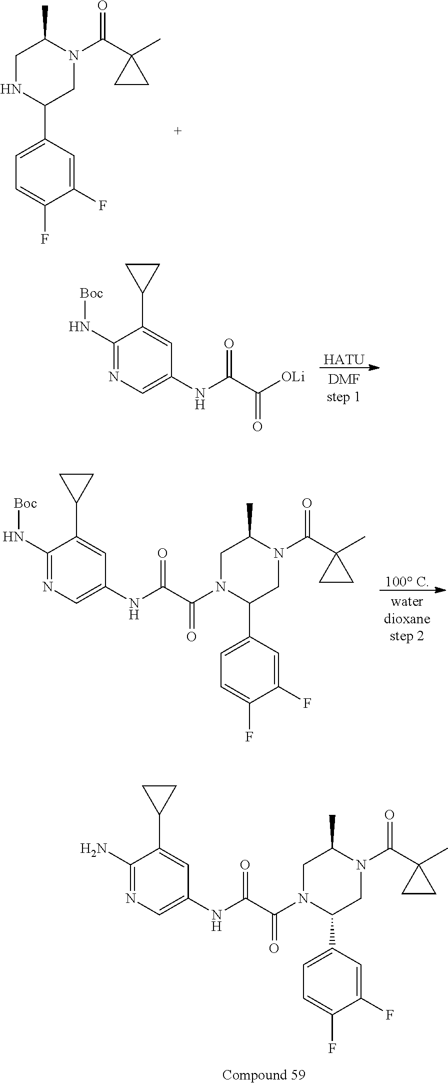

### Step 1: Synthesis of tert-butyl (3-cyclopropyl-5-(2-((2S,5R)-2-(3,4-difluorophenyl)-5-methyl-4-(1-methylcyclopropanecarbonyl)piperazin-1-yl)-2-oxoacetamido)pyridin-2-yl)carbamate

((2R)-5-(3,4-difluorophenyl)-2-methylpiperazin-1-yl)(1-methylcyclopropyl)methanone is prepared by Scheme 1.1 step 9. Yield: 0.55 g (78.42%).

LCMS(ESI): [M]⁺ m/z: calcd 282.2; found 283.2; Rt=0.943 min.

Prepared by Scheme 1.1, Step 10A. Yield: 0.32 g of crude.

LCMS(ESI): [M]⁺ m/z: calcd 597.2; found 598.2; Rt=1.250 min.

### Step 2: Synthesis of N-(6-amino-5-cyclopropylpyridin-3-yl)-2-((2S,5R)-2-(3,4-difluorophenyl)-5-methyl-4-(1-methylcyclopropanecarbonyl)piperazin-1-yl)-2-oxoacetamide (Compound 59)

Prepared by Boc Group Deprotection Method A. Yield: 83.3 mg (31.27%).

HPLC conditions: Column: YMC Triart C18 100*20 mm, 5 microM; 0-6 min 30-70% MeOH/water+0.1% NH₄OH 30 ml/min; (loading pump 4 ml/min MeOH).

Compound 59: ¹H NMR (600 MHz, DMSO-d₆) δ (ppm) 0.50 (m, 6H), 0.88 (m, 3H), 1.05 (s, 3H), 1.26 (m, 3H), 1.66 (m, 1H), 3.18 (m, 1H), 3.89 (m, 1H), 4.62 (m, 2H), 5.67 (m, 3H), 7.14 (m, 1H), 7.34 (m, 1H), 7.38 (m, 1H), 7.44 (m, 1H), 8.04 (m, 1H), 10.61 (m, 1H).

LCMS(ESI): [M]⁺ m/z: calcd 497.2; found 498.2; Rt=2.870 min.

### Example 85. Compound 152 N-(6-amino-5-cyclopropylpyridin-3-yl)-2-((2S,5R)-2-(4-fluorophenyl)-5-methyl-4-pivaloylpiperazin-1-yl)-2-oxoacetamide

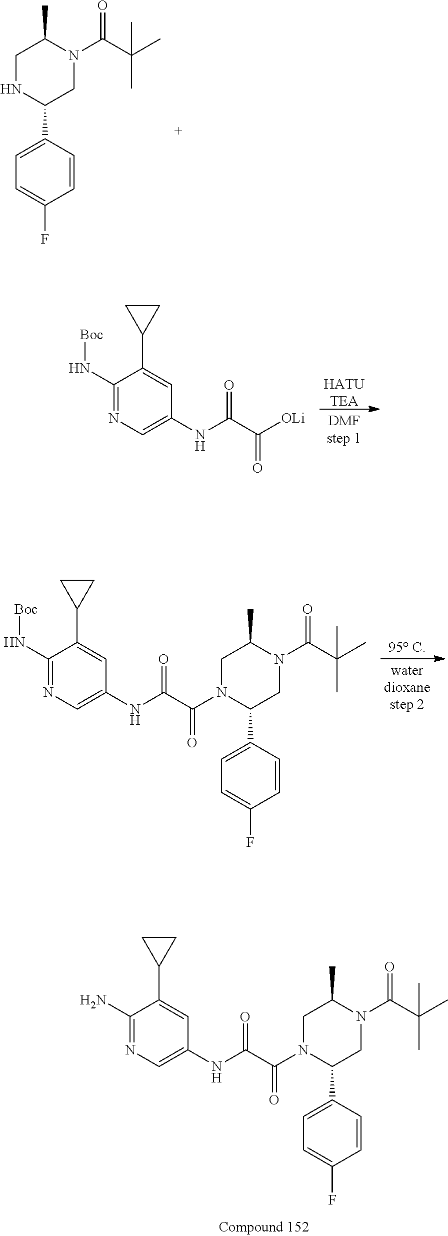

### Step 1: Synthesis of tert-butyl (3-cyclopropyl-5-(2-((2S,5R)-2-(4-fluorophenyl)-5-methyl-4-pivaloylpiperazin-1-yl)-2-oxoacetamido)pyridin-2-yl)carbamate

The synthesis of 1-((2R,5S)-5-(4-fluorophenyl)-2-methylpiperazin-1-yl)-2,2-dimethylpropan-1-one is prepared by the following procedure. tert-Butyl (2S,5R)-4-(2,2-dimethylpropanoyl)-2-(4-fluorophenyl)-5-methyl-piperazine-1-carboxylate (0.24 g, 634.12 μmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain 1-[(2R,5S)-5-(4-fluorophenyl)-2-methyl-piperazin-1-yl]-2,2-dimethyl-propan-1-one (0.17 g, 610.71 mol, 96.31% yield) which was used in the next step without further purification.

LCMS(ESI): [M]⁺ m/z: calcd 278.2; found 279.2; Rt=0.749 min.

Prepared by Scheme 1.4, step 9A. Yield: 130 mg (32.74%).

HPLC conditions: Column: Chromatorex C18 100*19 mm, 5 microM; 0-6 min 50-90% water/MeOH 30 ml/min (loading pump 4 ml MeOH).

LCMS(ESI): [M]⁺ m/z: calcd 581.2; found 582.2; Rt=3.809 min.

### Step 2: Synthesis of N-(6-amino-5-cyclopropylpyridin-3-yl)-2-((2S,5R)-2-(4-fluorophenyl)-5-methyl-4-pivaloylpiperazin-1-yl)-2-oxoacetamide (Compound 152)

Prepared by Boc Group Deprotection Method A. Yield: 49 mg (54.85%).

HPLC conditions: Column: YMC Triart C18 100*20 mm, 5 microM; 0-5 min 20-65% water/MeCN+0.1% NH₄OH 30 ml/min; (loading pump 4 ml/min MeCN).

Compound 152: ¹H NMR (600 MHz, DMSO-d₆) δ (ppm) 0.42-0.47 (m, 2H), 0.84-0.90 (m, 2H), 1.00-1.06 (m, 1H), 1.08 (s, 9H), 1.22-1.28 (m, 3H), 1.61-1.71 (m, 1H), 3.07-3.28 (m, 1H), 3.59-4.05 (m, 1H), 4.23-5.05 (m, 2H), 5.29-5.68 (m, 1H), 5.74-5.86 (m, 2H), 7.14-7.21 (m, 2H), 7.26-7.30 (m, 1H), 7.30-7.39 (m, 2H), 7.99-8.11 (m, 1H), 10.39-10.65 (m, 1H).

LCMS(ESI): [M]⁺ m/z: calcd 481.2; found 482.2; Rt=2.409 min.

### Example 86. Compound 74 N-(6-amino-5-methylpyridin-3-yl)-2-((2S,5R)-2-(3-chloro-4-fluorophenyl)-4-isobutyryl-5-methylpiperazin-1-yl)-2-oxoacetamide

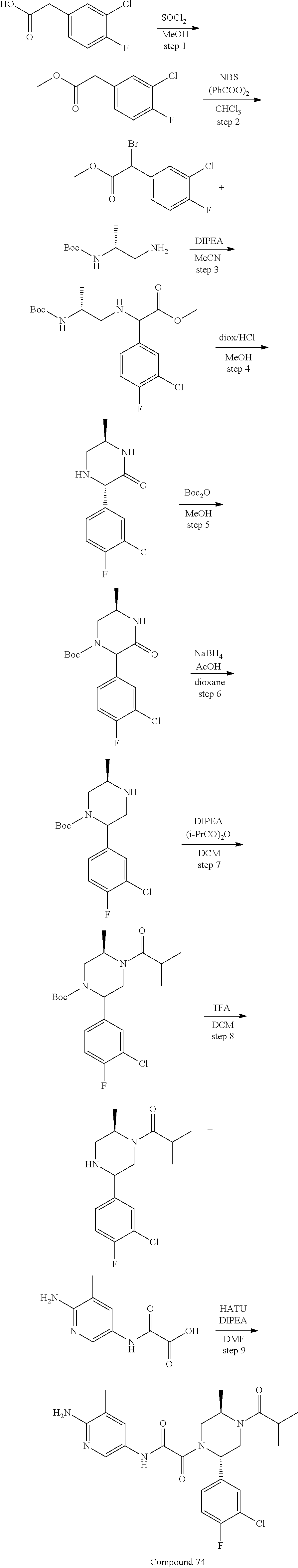

### Step 1: Synthesis of methyl 2-(3-chloro-4-fluorophenyl)acetate

Prepared by general procedure scheme 1.4 step 1. Yield: 10.17 g (99.64%).

¹H NMR (400 MHz, CDCl₃) δ (ppm) 3.54 (s, 2H), 3.67 (s, 3H), 7.07 (m, 1H), 7.12 (m, 1H), 7.30 (d, 1H).

### Step 2: Synthesis of methyl 2-bromo-2-(3-chloro-4-fluorophenyl)acetate

Prepared by general procedure scheme 1.4 step 2. Yield: 12.5 g (88.46%).

¹H NMR (400 MHz, CDCl₃) δ (ppm) 3.77 (s, 3H), 5.26 (s, 1H), 7.12 (m, 1H), 7.40 (m, 1H), 7.62 (d, 1H).

### Step 3: Synthesis of methyl 2-(((R)-2-((tert-butoxycarbonyl)amino)propyl)amino)-2-(3-chloro-4-fluorophenyl)acetate

Prepared by general procedure scheme 1.4 step 3. Yield: 5.8 g (49.78%).

CC conditions: The crude product was purified by silica gel with EtOAc/Hexane (1:2) as an eluent mixture.

LCMS(ESI): [M]⁺ m/z: calcd 374.2; found 375.2; Rt=1.144 min.

### Step 4: Synthesis of (3S,6R)-3-(3-chloro-4-fluorophenyl)-6-methylpiperazin-2-one

Prepared by general procedure scheme 1.4 step 4. Yield: 1.2 g (31.96%).

LCMS(ESI): [M]⁺ m/z: calcd 242.2; found 243.2; Rt=0.293 min.

### Step 5: Synthesis of (5R)-tert-butyl 2-(3-chloro-4-fluorophenyl)-5-methyl-3-oxopiperazine-1-carboxylate

Prepared by general procedure scheme 1.4 step 5. Yield: 1.6 g (94.39%).

LCMS(ESI): [M-t-Bu]⁺ m/z: calcd 286.2; found 287.2; Rt=1.185 min.

### Step 6: Synthesis of (5R)-tert-butyl 2-(3-chloro-4-fluorophenyl)-5-methylpiperazine-1-carboxylate

Prepared by general procedure scheme 1.4 step 6. Yield: 1.4 g of crude.

LCMS(ESI): [M-Boc]⁺ m/z: calcd 228.2; found 229.2; Rt=0.933 min.

### Step 7: Synthesis of (5R)-tert-butyl 2-(3-chloro-4-fluorophenyl)-4-isobutyryl-5-methylpiperazine-1-carboxylate

Prepared by general procedure scheme 1.4 step 7A. Yield: 0.27 g (89.02%).

LCMS(ESI): [M-t-Bu]⁺ m/z: calcd 342.2; found 343.2; Rt=1.503 min.

### Step 8: Synthesis of 1-((2R)-5-(3-chloro-4-fluorophenyl)-2-methylpiperazin-1-yl)-2-methylpropan-1-one

tert-Butyl (2S,5R)-2-(3-chloro-4-fluoro-phenyl)-5-methyl-4-(2-methylpropanoyl)piperazine-1-carboxylate (0.27 g, 676.86 mol) was dissolved in DCM (10 mL) and TFA (385.89 mg, 3.38 mmol, 260.74 L) was added to reaction mixture at 0° C. then the solution was allowed to warm to 25° C. and stirred for 16 hr. Reaction mixture was diluted with DCM (30 ml) and washed by saturated aqueous NaHCO₃ (3*30 ml). The resulting organic phase was dried over Na₂SO₄ and evaporated under reduced pressure to afford 1-[(2R,5S)-5-(3-chloro-4-fluoro-phenyl)-2-methyl-piperazin-1-yl]-2-methyl-propan-1-one (0.12 g, 401.63 μmol, 59.34% yield).

LCMS(ESI): [M]⁺ m/z: calcd 298.2; found 299.2; Rt=0.947 min.

### Step 9: Synthesis of N-(6-amino-5-methylpyridin-3-yl)-2-((2S,5R)-2-(3-chloro-4-fluorophenyl)-4-isobutyryl-5-methylpiperazin-1-yl)-2-oxoacetamide

Prepared by general procedure scheme 1.4 step 9B. Yield: 30.4 mg (15.90%).

HPLC conditions: Column: Chromarex C18 100*19 mm, 5 microM; 0-6 min 20-50% MeCN/water+0.2% FA 30 ml/min; (loading pump 4 ml/min MeCN).

Compound 74: ¹H NMR (DMSO-d₆, 600 MHz): δ (ppm) 0.73-1.09 (m, 6H), 1.24 (m, 3H), 1.98-2.03 (m, 3H), 2.67-2.82 (m, 1H), 3.13-3.20 (m, 1H), 3.61-4.51 (m, 4H), 4.87-4.92 (m, 1H), 5.32-5.66 (m, 3H), 7.23-7.52 (m, 4H), 8.03 (d, 1H), 10.52 (s, 1H).

LCMS(ESI): [M]⁺ m/z: calcd 475.2; found 476.2; Rt=2.864 min.

### Example 87. Compound 42 2-((2S,5R)-5-methyl-2-(3-(4-methylpiperazin-1-yl)phenyl)-4-pivaloylpiperazin-1-yl)-2-oxo-N-(1H-pyrazolo[4,3-c]pyridin-7-yl)acetamide

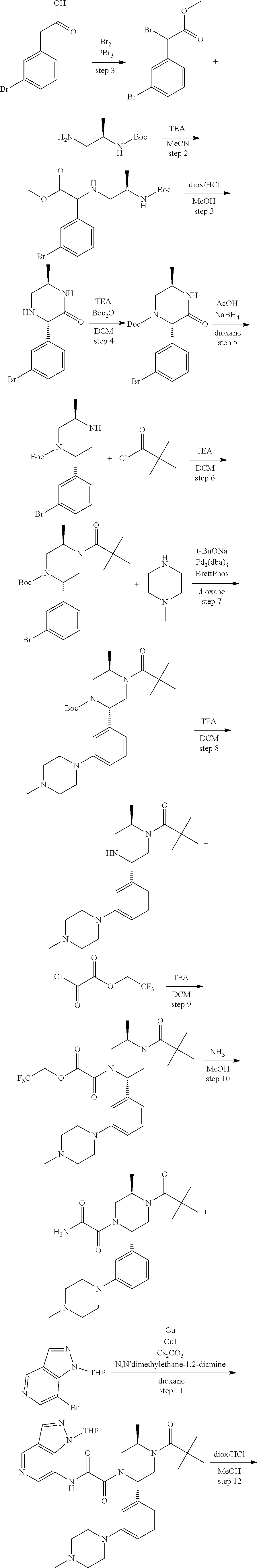

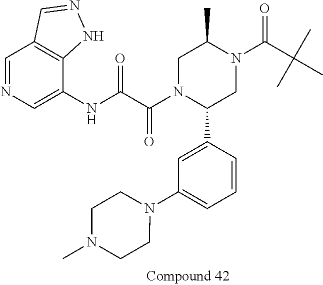 [SMILES: C[C@@H]1CN(C(=O)C(=O)Nc2cncc3cn[nH]c23)[C@@H](c2cccc(N3CCN(C)CC3)c2)CN1C(=O)C(C)(C)C]

### Step 1: Synthesis of methyl 2-bromo-2-(3-bromophenyl)acetate

To 2-(3-bromophenyl)acetic acid (5 g, 23.25 mmol) under argon was added phosphorus tribromide (18.88 g, 69.75 mmol, 6.56 mL) and the suspension stirred at room temperature for 45 minutes. Molecular bromine (18.58 g, 116.26 mmol, 5.99 mL) was added dropwise over 5 min. The mixture was stirred at 100° C. for 3 hr and then cooled. Anhydrous MeOH (20 mL) was added dropwise over 30 minutes, and then the reaction mixture was diluted with MTBE (200 mL), washed with 5% NaHCO₃ (400 mL), brine (100 mL), and then dried over Na₂SO₄. The mixture was filtered and concentrated in vacuum to afford methyl 2-bromo-2-(3-bromophenyl)acetate (7.25 g, crude).

¹H NMR (400 MHz, CDCl₃) δ (ppm) 3.78 (s, 3H), 5.26 (s, 1H), 7.22 (t, 1H), 7.45 (d, 2H), 7.68 (s, 1H).

### Step 2: Synthesis of methyl 2-(3-bromophenyl)-2-(((R)-2-((tert-butoxycarbonyl)amino)propyl)amino)acetate

Prepared by general procedure scheme 1.4 step 3. Yield: 6.7 g (70.92%).

LCMS(ESI): [M]⁺ m/z: calcd 401.2; found 402.2; Rt=0.944 min.

### Step 3: Synthesis of (3S,6R)-3-(3-bromophenyl)-6-methylpiperazin-2-one

Prepared by general procedure scheme 1.4 step 4. Yield: 1.05 g (23.37%).

LCMS(ESI): [M]⁺ m/z: calcd 269.2; found 270.2; Rt=0.566 min.

### Step 4: Synthesis of (2S,5R)-tert-butyl 2-(3-bromophenyl)-5-methyl-3-oxopiperazine-1-carboxylate

Prepared by general procedure scheme 1.4 step 5. Yield: 1.7 g of crude.

LCMS(ESI): [M-t-Bu]⁺ m/z: calcd 313.2; found 314.2; Rt=2.324 min.

### Step 5: Synthesis of (2S,5R)-tert-butyl 2-(3-bromophenyl)-5-methylpiperazine-1-carboxylate

Prepared by general procedure scheme 1.4 step 6. Yield: 1.7 g of crude.

LCMS(ESI): [M]⁺ m/z: calcd 355.2; found 356.2; Rt=1.086 min.

### Step 6: Synthesis of (2S,5R)-tert-butyl 2-(3-bromophenyl)-5-methyl-4-pivaloylpiperazine-1-carboxylate

Prepared by general procedure scheme 1.4 step 7A. Yield: 1.15 g (77.49%).

LCMS(ESI): [M-t-Bu]⁺ m/z: calcd 383.2; found 384.2; Rt=1.602 min.

### Step 7: Synthesis of (2S,5R)-tert-butyl 5-methyl-2-(3-(4-methylpiperazin-1-yl)phenyl)-4-pivaloylpiperazine-1-carboxylate

tert-Butyl (2S,5R)-2-(3-bromophenyl)-4-(2,2-dimethylpropanoyl)-5-methyl-piperazine-1-carboxylate (1.15 g, 2.62 mmol), 1-methylpiperazine (262.15 mg, 2.62 mmol, 290.31 μL), sodium tert-butoxide (503.04 mg, 5.23 mmol) and BrettPhos (140.49 mg, 261.73 mol) were mixed together in dioxane (20 mL) and the resulting mixture was evacuated and backfilled three times with argon. tris(Dibenzylideneacetone)dipalladium (O) (119.84 mg, 130.86 mol) was added to the previous mixture and the resulting mixture was heated at 100° C. (oil bath) overnight. The reaction mixture was cooled and filtered; the filtrate was concentrated in vacuum. The obtained product was used in the next step without further purification. tert-Butyl (2S,5R)-4-(2,2-dimethylpropanoyl)-5-methyl-2-[3-(4-methylpiperazin-1-yl)phenyl]piperazine-1-carboxylate (1.15 g, 2.51 mmol, 95.80% yield) was obtained as a brown gum.

LCMS(ESI): [M]⁺ m/z: calcd 458.2; found 459.2; Rt=1.013 min.

### Step 8: Synthesis of 2,2-dimethyl-1-((2R,5S)-2-methyl-5-(3-(4-methylpiperazin-1-yl)phenyl)piperazin-1-yl)propan-1-one

tert-Butyl (2S,5R)-4-(2,2-dimethylpropanoyl)-5-methyl-2-[3-(4-methylpiperazin-1-yl)phenyl]piperazine-1-carboxylate (1.15 g, 2.51 mmol) was dissolved in TFA (10 mL) and DCM (10 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated. The obtained product was used in the next step without further purification. 2,2-Dimethyl-1-[(2R,5S)-2-methyl-5-[3-(4-methylpiperazin-1-yl)phenyl]piperazin-1-yl]propan-1-one (0.9 g, crude) was obtained as a brown gum.

LCMS(ESI): [M]⁺ m/z: calcd 358.2; found 359.2; Rt=0.699 min.

### Step 9: Synthesis of 2,2,2-trifluoroethyl 2-((2S,5R)-5-methyl-2-(3-(4-methylpiperazin-1-yl)phenyl)-4-pivaloylpiperazin-1-yl)-2-oxoacetate

2,2,2-Trifluoroethyl 2-chloro-2-oxo-acetate (281.62 mg, 1.48 mmol) was added dropwise to a solution of 2,2-dimethyl-1-[(2R,5S)-2-methyl-5-[3-(4-methylpiperazin-1-yl)phenyl]piperazin-1-yl]propan-1-one (0.53 g, 1.48 mmol) and TEA (448.77 mg, 4.43 mmol, 618.14 L) in DCM (19.97 mL) at 0° C. After addition was complete, cooling bath was removed and resulting mixture was allowed to warm up to 20° C. and stirred for 12 hr. The reaction mixture was washed with water, dried over Na₂SO₄ and concentrated under reduced pressure. The obtained product was used in the next step without further purification. 2,2,2-Trifluoroethyl 2-oxo-2-[(2S,5R)-4-(2,2-dimethylpropanoyl)-5-methyl-2-[3-(4-methylpiperazin-1-yl)phenyl]piperazin-1-yl]acetate (0.65 g, 1.27 mmol, 85.78% yield) was obtained as a red gum.

LCMS(ESI): [M]⁺ m/z: calcd 512.2; found 513.2; Rt=1.094 min.

### Step 10: Synthesis of 2-((2S,5R)-5-methyl-2-(3-(4-methylpiperazin-1-yl)phenyl)-4-pivaloylpiperazin-1-yl)-2-oxoacetamide

2,2,2-Trifluoroethyl 2-oxo-2-[(2S,5R)-4-(2,2-dimethylpropanoyl)-5-methyl-2-[3-(4-methylpiperazin-1-yl)phenyl]piperazin-1-yl]acetate (0.65 g, 1.27 mmol) was dissolved in MeOH/NH₃ (10 mL) The reaction mixture was stirred overnight. The reaction mixture was evaporated to dryness. The reaction mixture was submitted to HPLC (2-10 min 30-45% MeOH+NH₃ flow 30 ml/min (loading pump 4 ml MeOH), column: SunFire C18). 2-Oxo-2-[(2S,5R)-4-(2,2-dimethylpropanoyl)-5-methyl-2-[3-(4-methylpiperazin-1-yl)phenyl]piperazin-1-yl]acetamide (0.09 g, 209.52 μmol, 16.52% yield) was obtained as ared gum.

LCMS(ESI): [M]⁺ m/z: calcd 429.2; found 430.2; Rt=0.759 min.

### Step 11: Synthesis of 2-((2S,5R)-5-methyl-2-(3-(4-methylpiperazin-1-yl)phenyl)-4-pivaloylpiperazin-1-yl)-2-oxo-N-(1-(tetrahydro-2H-pyran-2-yl)-1H-pyrazolo[4,3-c]pyridin-7-yl)acetamide

2-Oxo-2-[(2S,5R)-4-(2,2-dimethylpropanoyl)-5-methyl-2-[3-(4-methylpiperazin-1-yl)phenyl]piperazin-1-yl]acetamide (0.09 g, 209.52 μmol), 7-bromo-1-tetrahydropyran-2-yl-pyrazolo[4,3-c]pyridine (65.02 mg, 230.47 μmol), copper (I) iodide (39.90 mg, 209.52 mol, 7.10 μL), cesium carbonate (136.53 mg, 419.04 mol) and (¹R,²R)—N¹,N²-dimethylcyclohexane-1,2-diamine (44.70 mg, 314.28 μmol) were mixed in dioxane (3 mL) under argon, and then stirred overnight at 100° C. for 12 hr in vial. The reaction mixture was submitted to HPLC (2-10min20-45% water/MeOH+NH₃ 30 ml/min; loading pump 4 ml/min MeOH+NH₃ column SunFire 19*100 mm). 2-Oxo-2-[(2S,5R)-4-(2,2-dimethylpropanoyl)-5-methyl-2-[3-(4-methylpiperazin-1-yl)phenyl]piperazin-1-yl]-N-(1-tetrahydropyran-2-ylpyrazolo[4,3-c]pyridin-7-yl) acetamide (0.017 g, 26.95 μmol, 12.86% yield) was obtained as a light-yellow solid.

LCMS(ESI): [M]⁺ m/z: calcd 630.2; found 631.2; Rt=0.929 min.

### Step 12: Synthesis of 2-((2S,5R)-5-methyl-2-(3-(4-methylpiperazin-1-yl)phenyl)-4-pivaloylpiperazin-1-yl)-2-oxo-N-(1H-pyrazolo[4,3-c]pyridin-7-yl)acetamide (Compound 42)

2-Oxo-2-[(2S,5R)-4-(2,2-dimethylpropanoyl)-5-methyl-2-[3-(4-methylpiperazin-1-yl)phenyl]piperazin-1-yl]-N-(1-tetrahydropyran-2-ylpyrazolo[4,3-c]pyridin-7-yl)acetamide (0.017 g, 26.95 μmol) was dissolved in a mixture of MeOH (2 mL) and dioxane/HCl (2 mL). The resulting clear solution was stirred for 12 hr at 20° C. The reaction mixture was concentrated on vacuum. 2-Oxo-N-(1H-pyrazolo[4,3-c]pyridin-7-yl)-2-[(2S,5R)-4-(2,2-dimethylpropanoyl)-5-methyl-2-[3-(4-methylpiperazin-1-yl)phenyl]piperazin-1-yl]acetamide (0.017 g, 25.91 mol, 96.15% yield, 3HCl) was obtained as a light-yellow solid. Compound 42: ¹H NMR (600 MHz, DMSO-d₆) δ (ppm) 1.01-1.14 (m, 9H), 1.24-1.37 (m, 3H), 2.77-2.82 (m, 3H), 2.96-3.16 (m, 6H), 3.61-4.07 (m, 6H), 4.33-5.70 (m, 2H), 6.83-7.36 (m, 4H), 8.47-10.45 (m, 4H), 10.72-11.02 (m, 1H), 11.93-12.42 (m, 1H), 14.62-15.13 (m, 1H).

LCMS(ESI): [M]⁺ m/z: calcd 546.2; found 547.2; Rt=2.129 min.

### Example 88. Compound 112 N-(6-amino-5-cyclopropylpyridin-3-yl)-2-((2S,5R)-2-(4-fluorophenyl)-5-methyl-4-(1-(trifluoromethyl)cyclopropanecarbonyl)piperazin-1-yl)-2-oxoacetamide

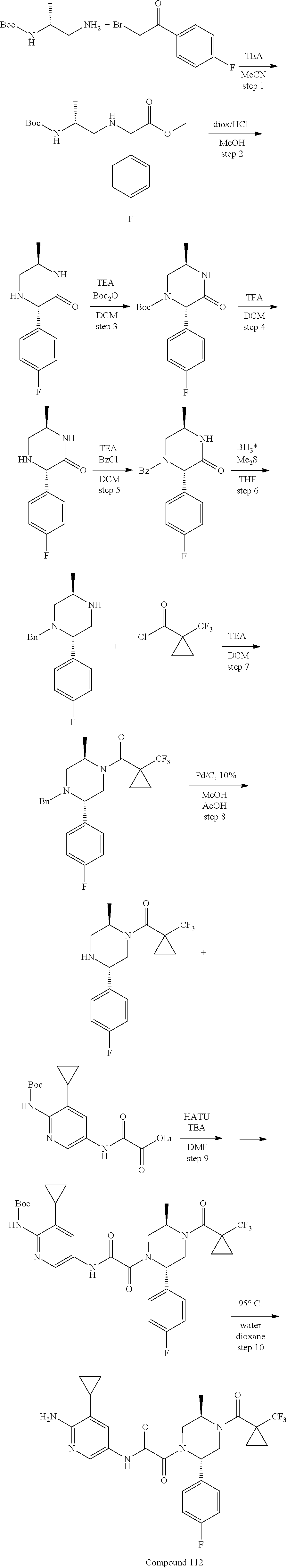

### Step 1: Synthesis of methyl 2-(((R)-2-((tert-butoxycarbonyl)amino)propyl)amino)-2-(4-fluorophenyl)acetate

Prepared by general procedure scheme 1.4 step 3. Yield: 70 g of crude.

LCMS(ESI): [M]⁺ m/z: calcd 340.2; found 341.2; Rt=1.036 min.

### Step 2: Synthesis of (3S,6R)-3-(4-fluorophenyl)-6-methylpiperazin-2-one

Prepared by general procedure scheme 1.4 step 4. Yield: 13.2 g (30.83%).

LCMS(ESI): [M]⁺ m/z: calcd 208.2; found 209.2; Rt=0.238 min.

### Step 3: Synthesis of (2S,5R)-tert-butyl 2-(4-fluorophenyl)-5-methyl-3-oxopiperazine-1-carboxylate

Prepared by general procedure scheme 1.4 step 5. Yield: 19.3 g (98.74%).

LCMS(ESI): [M-t-Bu]⁺ m/z: calcd 252.2; found 253.2; Rt=1.183 min.

### Step 4: Synthesis of (3S,6R)-3-(4-fluorophenyl)-6-methylpiperazin-2-one

Prepared by general procedure Boc-deprotection Method D. Yield: 2.1 g of crude (TFA-salt).

LCMS(ESI): [M]⁺ m/z: calcd 208.2; found 209.2; Rt=0.238 min.

### Step 5: Synthesis of (3S,6R)-4-benzoyl-3-(4-fluorophenyl)-6-methylpiperazin-2-one

Benzoyl chloride (1.10 g, 7.84 mmol) was added dropwise to a solution of (3S,6R)-3-(4-fluorophenyl)-6-methyl-piperazin-2-one (2.1 g, 6.54 mmol, TFA) and TEA (1.98 g, 19.61 mmol, 2.73 mL) in DCM (30 mL) and resulting mixture was stirred at 20° C. for 2 hr. Then, 20% aq. K₂CO₃ solution (20 ml) was added and stirring was continued for 10 min. After that, organic layer was separated, dried over K₂CO₃ and concentrated under reduced pressure, affording (3S, 6R)-4-benzoyl-3-(4-fluorophenyl)-6-methyl-piperazin-2-one (2.33 g, crude).

LCMS(ESI): [M]⁺ m/z: calcd 312.2; found 313.2; Rt=1.047 min.

### Step 6: Synthesis of (2S,5R)-1-benzyl-2-(4-fluorophenyl)-5-methylpiperazine

Borane dimethyl sulfide complex (2.66 g, 35.06 mmol, 3.33 mL) was added dropwise to the solution of (3S,6R)-4-benzoyl-3-(4-fluorophenyl)-6-methyl-piperazin-2-one (2.19 g, 7.01 mmol) in THF (30 mL). Resulting mixture was stirred at 65° C. for 18 hr. Then, it was cooled to rt and excess of borane was destroyed by dropwise addition of MeOH (10 ml). After H₂ evolution ceased, volatiles were removed under reduced pressure and residue was taken up in 2M aq. HCl (40 ml) and stirred at 50° C. for 40 minutes. Resulting cloudy solution was filtered and extracted with DCM (2×10 ml). DCM layers were discarded and aqueous layer was basified to pH≈11 with solid potassium hydroxide. Precipitated amine was extracted with DCM (2×25 ml). Organic layers were separated, dried over K₂CO₃ and concentrated in vacuum, affording (2S,5R)-1-benzyl-2-(4-fluorophenyl)-5-methyl-piperazine (1.31 g, 4.61 mmol, 65.70% yield).

LCMS(ESI): [M]⁺ m/z: calcd 284.2; found 285.2; Rt=1.049 min.

### Step 7: Synthesis of ((2R,5S)-4-benzyl-5-(4-fluorophenyl)-2-methylpiperazin-1-yl)(1-(trifluoromethyl)cyclopropyl)methanone

1-(Trifluoromethyl)cyclopropanecarbonyl chloride (1.03 g, 5.94 mmol) was added dropwise to the solution of (2S,5R)-1-benzyl-2-(4-fluorophenyl)-5-methyl-piperazine (1.3 g, 4.57 mmol) and TEA (925.18 mg, 9.14 mmol, 1.27 mL) in DCM (30 mL). Resulting mixture was stirred at 20° C. for 2 hr. Then, 15% aq. K₂CO₃ solution (20 ml) was added and stirring was continued for 5 min. After that, organic layer was separated, dried over K₂CO₃ and concentrated under reduced pressure, affording [(2R,5S)-4-benzyl-5-(4-fluorophenyl)-2-methyl-piperazin-1-yl]-[1-(trifluoromethyl)cyclopropyl]methanone (1.93 g, crude).

LCMS(ESI): [M]⁺ m/z: calcd 420.2; found 421.2; Rt=1.606 min.

### Step 8: Synthesis of ((2R,5S)-5-(4-fluorophenyl)-2-methylpiperazin-1-yl)(1-(trifluoromethyl)cyclopropyl)methanone

Palladium, 10% on carbon (0.3 g, 281.90 umol, 10% purity) was added to the solution of [(2R,5S)-4-benzyl-5-(4-fluorophenyl)-2-methyl-piperazin-1-yl]-[1-(trifluoromethyl)cyclopropyl]methanone (1.93 g, 4.59 mmol) in MeOH (30 mL) and acetic acid (10 mL). Reaction flask was evacuated and backfilled with hydrogen (138.80 mg, 68.86 mmol) from attached balloon. Resulting mixture was stirred at 50° C. for 16 hr. Then, catalyst was filtered off and filtrate was concentrated under reduced pressure. Residue was partitioned between 10% aq. K₂CO₃ solution (20 ml) and DCM (40 ml). Organic layer was separated, dried over K₂CO₃ and concentrated in vacuum, affording [(2R,5S)-5-(4-fluorophenyl)-2-methyl-piperazin-1-yl]-[1-(trifluoromethyl)cyclopropyl]methanone (1.49 g, 4.51 mmol, 98.27% yield)

LCMS(ESI): [M]⁺ m/z: calcd 330.2; found 331.2; Rt=0.789 min.

### Step 9: Synthesis of tert-butyl (3-cyclopropyl-5-(2-((2S,5R)-2-(4-fluorophenyl)-5-methyl-4-(1-(trifluoromethyl)cyclopropanecarbonyl)piperazin-1-yl)-2-oxoacetamido)pyridin-2-yl)carbamate

Prepared by general procedure scheme 1.1 step 1OA. Yield: 0.1 g (19.10%).

HPLC conditions: Column: YMC Triart C18 100*20 mm, 5 microM; 0-5 min 45-80% MeOH/water+FA 40 ml/min; (loading pump 4 ml/min MeOH).

LCMS(ESI): [M]⁺ m/z: calcd 633.2; found 634.2; Rt=3.753 min.

### Step 10: Synthesis of N-(6-amino-5-cyclopropylpyridin-3-yl)-2-((2S,5R)-2-(4-fluorophenyl)-5-methyl-4-(1-(trifluoromethyl)cyclopropanecarbonyl)piperazin-1-yl)-2-oxoacetamide (Compound 112)

Prepared by Boc Group Deprotection Method A. Yield: 62 mg (73.63%).

HPLC conditions: Column: YMC Triart C18 100*20 mm, 5 microM; 0-5 min 40-80% MeOH/water+0.1% NH₄OH 30 ml/min; (loading pump 4 ml/min MeOH).

Compound 112: ¹H NMR (600 MHz, DMSO-d₆) δ (ppm) 0.41-0.47 (m, 2H), 0.85-0.89 (m, 2H), 0.95-1.02 (m, 1H), 1.10-1.19 (m, 2H), 1.22-1.35 (m, 4H), 1.60-1.69 (m, 1H), 2.51-3.12 (m, 1H), 3.32-3.77 (m, 1H), 3.83-4.15 (m, 1H), 4.24-5.06 (m, 2H), 5.37-5.76 (m, 1H), 5.76-5.82 (m, 2H), 7.12-7.20 (m, 2H), 7.23-7.31 (m, 1H), 7.33-7.46 (m, 2H), 7.96-8.18 (m, 1H), 10.31-10.70 (m, 1H).

LCMS(ESI): [M]⁺ m/z: calcd 533.2; found 534.2; Rt=2.706 min.

### Example 89. Compound 121 N-(6-amino-5-ethylpyridin-3-yl)-2-((2S,5R)-5-methyl-4neopentyl-2-phenylpiperazin-1-yl)-2-oxoacetamide

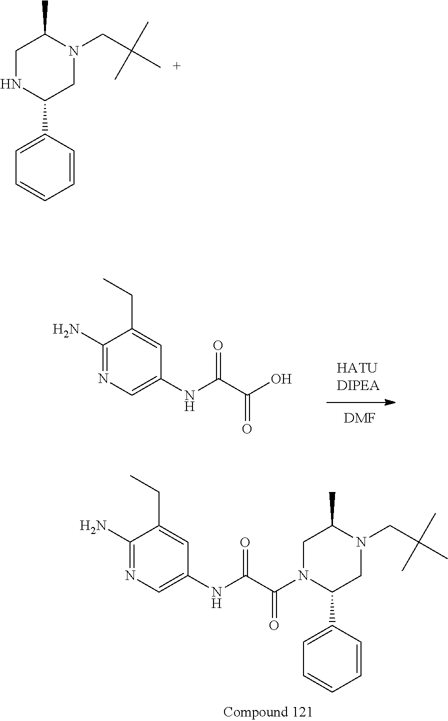

2,2-dimethyl-1-((2R,5S)-2-methyl-5-phenylpiperazin-1-yl)propan-1-one is prepared by Scheme 1.4 step 8. Yield: 0.46 g (97.98%).

LCMS(ESI): [M]⁺ m/z: calcd 260.2; found 261.2; Rt=0.910 min.

(2R,5S)-1-(2,2-Dimethylpropyl)-2-methyl-5-phenyl-piperazine (0.14 g, 568.20 mol) and 2-[(6-amino-5-ethyl-3-pyridyl)amino]-2-oxo-acetic acid (118.87 mg, 568.20 mol) and DIPEA (220.31 mg, 1.70 mmol, 296.91 L) were mixed in dry DMF (5 mL) at 25° C. and the resulting mixture was stirred for 5 min. HATU (280.86 mg, 738.67 mol) was added thereto and the resulting mixture was stirred at rt for 16 hr. The solvent was evaporated under reduced pressure and the crude was purified by reverse phase HPLC (column: YMC Triart C18 100×20 mm, 5 um; mobile phase: 30-30-80% 0-1-6 min H₂O/MeCN/0.1% NH₄OH, flow: 30 ml/min (loading pump 4 ml/min MeCN)). Then the product was purified second time. The conditions of second reverse phase HPLC (column: YMC Triart C18 100×20 mm, 5 um; mobile phase: 50-50-85% 0-1-6 min H₂O/MeOH/0.1% NH₄OH, flow: 30 ml/min (loading pump 4 ml/min MeOH)) to afford the more pure product N-(6-amino-5-ethyl-3-pyridyl)-2-[(2S,5R)-4-(2,2-dimethylpropyl)-5-methyl-2-phenyl-piperazin-1-yl]-2-oxo-acetamide (0.0516 g, 117.92 mol, 20.75% yield).

Compound 121:

1H NMR (500 MHz, DMSO-d₆) δ (ppm) 0.73 (s, 9H), 0.97-1.02 (m, 3H), 1.07-1.13 (m, 3H), 1.90-1.99 (m, 1H), 2.16-2.22 (m, 1H), 2.35-2.40 (m, 2H), 2.86-2.99 (m, 1H), 3.03-3.08 (m, 1H), 3.09-3.19 (m, 1H), 3.52-4.13 (m, 2H), 5.08-5.43 (m, 1H), 5.60-5.67 (m, 2H), 7.22-7.28 (m, 1H), 7.30-7.36 (m, 2H), 7.45-7.56 (m, 3H), 7.98-8.07 (m, 1H), 10.36-10.53 (m, 1H).

LCMS(ESI): [M]⁺ m/z: calcd 437.2; found 438.2; Rt=2.404 min.

### Example 90. Compound 63 5-(2-((2S,5R)-2-(3-chloro-4-fluorophenyl)-5-methyl-4-(1-(trifluoromethyl)cyclopropanecarbonyl)piperazin-1-yl)-2-oxoacetamido)-2-methoxynicotinamide

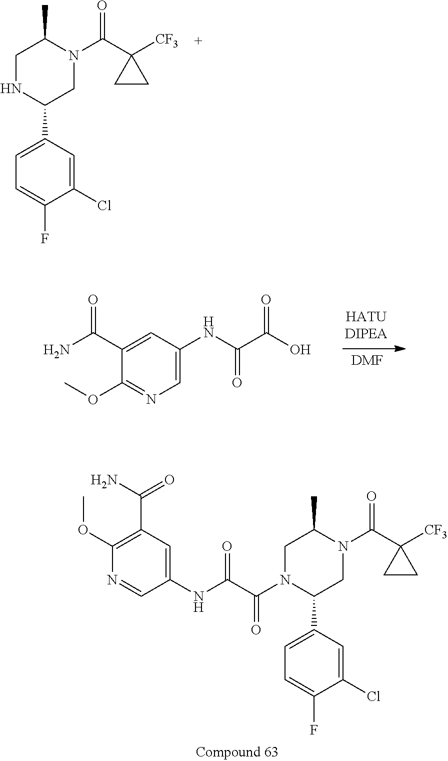

The synthesis of ((2R,5S)-5-(3-chloro-4-fluorophenyl)-2-methylpiperazin-1-yl)(1-(trifluoromethyl)cyclopropyl)methanone is given by the following procedure. tert-Butyl (2S,5R)-2-(3-chloro-4-fluoro-phenyl)-5-methyl-4-[1-(trifluoromethyl)cyclopropane carbonyl]piperazine-1-carboxylate (0.29 g, 623.82 μmol) was dissolved in DCM (30.23 mL) and TFA (355.65 mg, 3.12 mmol, 240.30 L) was added to reaction mixture at OoC then the solution was allowed to warm to 25° C. and stirred at 25° C. for 16 hr. Reaction mixture was diluted with DCM (30 ml.) and washed by saturated aqueous NaHCO₃ (3*30 ml). The resulting organic phase was dried over Na₂SO₄ and evaporated under reduced pressure to afford [(2R,5S)-5-(3-chloro-4-fluoro-phenyl)-2-methyl-piperazin-1-yl]-[1-(trifluoromethyl)cyclopropyl]methanone (0.18 g, 493.47 mol, 79.10% yield).

LCMS(ESI): [M]⁺ m/z: calcd 364.2; found 365.2; Rt=1.044 min.

Prepared by Scheme 1.4 step 9B. Yield: 46.9 mg (11.23%).

HPLC conditions: Column: Chromarex C18 100*19 mm, 5 microM; 0-5 min 25-50% MeCN/water+0.1% FA 30 ml/min; (loading pump 4 ml/min MeCN).

1H NMR (600 MHz, dmso) 6 0.98-1.37 (m, 8H), 2.62-3.12 (m, 1H), 3.71-3.97 (m, 4H), 4.09-5.84 (m, 3H), 7.22-7.51 (m, 3H), 7.70-7.78 (m, 2H), 8.46-8.59 (m, 2H), 10.93-11.26 (m, 1H).

LCMS(ESI): [M]⁺ m/z: calcd 585.2; found 586.2; Rt=3.214 min.

### Example 91. Compound 71 N-(6-amino-5-ethylpyridin-3-yl)-2-((2S,5R)-4-isobutyryl-5-methyl-2-(p-tolyl)piperazin-1-yl)-2-oxoacetamide

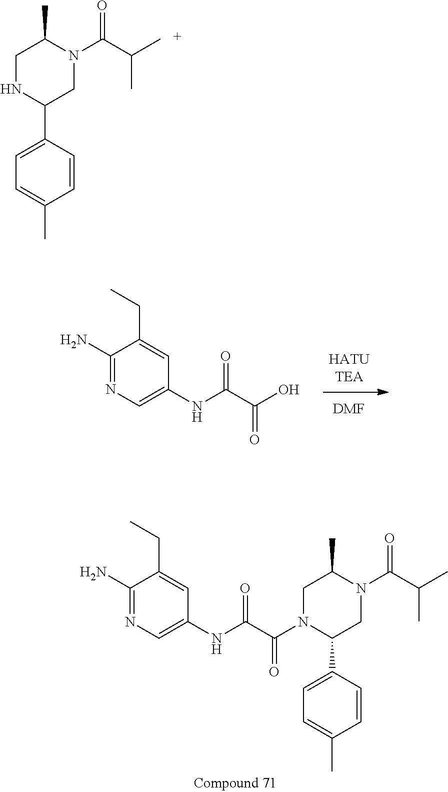

The synthesis of 2-methyl-1-((2R)-2-methyl-5-(p-tolyl)piperazin-1-yl)propan-1-one is given by the following procedure. tert-Butyl (2S,5R)-5-methyl-4-(2-methylpropanoyl)-2-(p-tolyl)piperazine-1-carboxylate (568 mg, 1.58 mmol) was dissolved in DCM (5 mL).TFA (2.69 g, 23.63 mmol, 1.82 mL) was added dropwise to the resulting solution and stirred for 2 hr. The mixture was then basified with potassium carbonate (5 g in 10 ml H₂O), washed with DCM (2×5 ml). The organic layer was combined, washed with water, dried over Na₂SO₄ and evaporated to give 2-methyl-1-[(2R,5S)-2-methyl-5-(p-tolyl)piperazin-1-yl]propan-1-one (318 mg, 1.22 mmol, 77.51% yield).

LCMS(ESI): [M]⁺ m/z: calcd 260.2; found 261.2; Rt=0.933 min.

Prepared by Scheme 1.4, step 9A. Yield: 74.8 mg (30.81%).

HPLC conditions: Column: SunFire C18 100*19 mm, 5 microM; 2-10 min 0-50% MeOH+NH₃; (loading pump 4 ml/min MeOH).

Compound 71: ¹H NMR (600 MHz, DMSO-d₆)₆ (ppm) 6 0.77-0.89 (m, 3H), 0.91-1.00 (m, 3H), 1.07-1.27 (m, 6H), 2.22-2.28 (m, 3H), 2.35-2.42 (m, 2H), 2.66-2.82 (m,

1.4H), 3.09-3.22 (m, 1H), 3.49-3.63 (m, 0.6H), 3.64-4.06 (m, 1H), 4.13-4.34 (m, 1H), 4.41-4.97 (m, 1H), 5.24-5.62 (m, 1H), 5.63-5.70 (m, 2H), 7.10-7.20 (m, 3H), 7.20-7.25 (m, 1H), 7.28-7.53 (m, 1H), 7.86-8.08 (m, 1H), 10.34-10.64 (m, 1H).

LCMS(ESI): [M]⁺ m/z: calcd 451.2; found 452.2; Rt=1.078 min.

### Example 92. Compound 131 N-(6-amino-5-methylpyridin-3-yl)-2-((5R)-2-(4-chlorophenyl)-5-methyl-4-(1-(trifluoromethyl)cyclopropanecarbonyl)piperazin-1-yl)-2-oxoacetamide

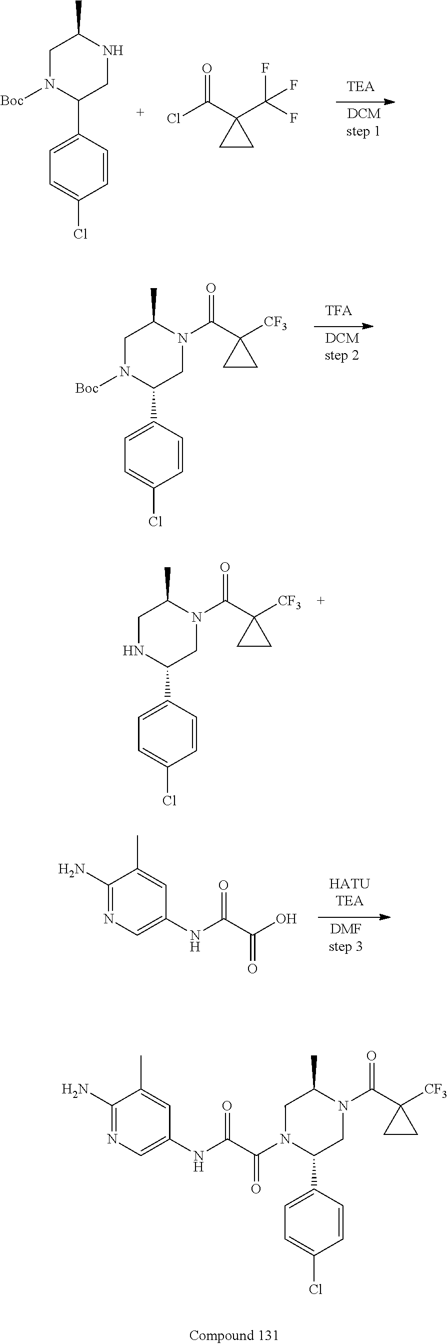

### Step 1: Synthesis of (2S,5R)-tert-butyl 2-(4-chlorophenyl)-5-methyl-4-(1-(trifluoromethyl)cyclopropanecarbonyl)piperazine-1-carboxylate

The synthesis of (5R)-tert-butyl 2-(4-chlorophenyl)-5-methylpiperazine-1-carboxylate is prepared by Scheme 1.4 step 6. Yield: 1.2 g of crude.

LCMS(ESI): [M-Boc]⁺ m/z: calcd 210.2; found 211.2; Rt=1.082 min.

Prepared by Scheme 1.4 step 7A. Yield: 0.35 g of crude.

LCMS(ESI): [M-t-Bu]⁺ m/z: calcd 390.2; found 391.2; Rt=1.528 min.

### Step 2: Synthesis of ((2R,5S)-5-(4-chlorophenyl)-2-methylpiperazin-1-yl)(1-(trifluoromethyl)cyclopropyl)methanone

tert-Butyl (5R)-2-(4-chlorophenyl)-5-methyl-4-[1-(trifluoromethyl)cyclopropanecarbonyl]piperazine-1-carboxylate (0.35 g, 783.19 μmol) was dissolved in DCM (10 mL), TFA (1.79 g, 15.66 mmol, 1.21 mL) was added in one portion and the resulting mixture was stirred at 25° C. for 20 hr. Upon completion, solvent was evaporated; 10 ml of water was added to the residue. Aqueous layer was basified with K₂CO₃ to alkaline pH. Mixture was extracted with DCM (3*10 ml), combined organic layers were dried over Na₂SO₄, filtered and concentrated in vacuum, to afford [(2R)-5-(4-chlorophenyl)-2-methyl-piperazin-1-yl]-[1-(trifluoromethyl)cyclopropyl]methanone (0.2 g, 576.74 μmol, 73.64% yield).

LCMS(ESI): [M]⁺ m/z: calcd 346.2; found 347.2; Rt=0.753 min.

### Step 3: Synthesis of N-(6-amino-5-methylpyridin-3-yl)-2-((5R)-2-(4-chlorophenyl)-5-methyl-4-(1-(trifluoromethyl)cyclopropanecarbonyl)piperazin-1-yl)-2-oxoacetamide (Compound 131)

Prepared by Scheme 1.4 step 9A. Yield: 27.9 mg (9.23%).

HPLC conditions: Column: XBridge C18 100*19 mm, 5 microM; 0-5 min 15-65% MeCN/water+0.1% NH₄OH 30 ml/min; (loading pump 4 ml/min MeOH).

Compound 131: 1H NMR (600 MHz, dmso) 6 0.93-1.02 (m, 1H), 1.11-1.33 (m, 7H), 1.98-2.04 (m, 3H), 2.61-3.09 (m, 1H), 3.64-4.97 (m, 3H), 5.38-5.80 (m, 3H), 7.16-7.27 (m, 1H), 7.31-7.36 (m, 1H), 7.38-7.43 (m, 2H), 7.46-7.55 (m, 1H), 7.99-8.10 (m, 1H), 10.38-10.73 (m, 1H).

LCMS(ESI): [M]⁺ m/z: calcd 523.2; found 524.2; Rt=2.616 min.

### Example 93. Compound 85 N-(6-amino-5-ethylpyridin-3-yl)-2-((2S,5R)-5-methyl-2-(p-tolyl)-4-(1-(trifluoromethyl)cyclopropanecarbonyl)piperazin-1-yl)-2-oxoacetamide

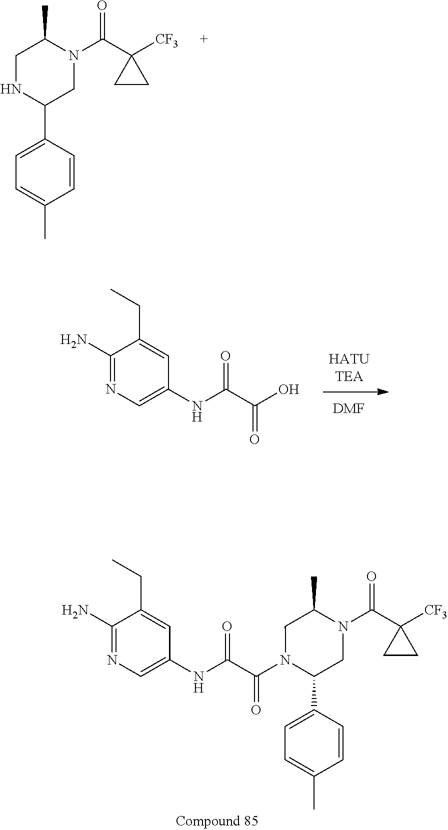

The synthesis of ((2R)-2-methyl-5-(p-tolyl)piperazin-1-yl)(1-(trifluoromethyl)cyclopropyl)methanone is given by the following procedure. tert-Butyl (2S,5R)-5-methyl-2-(p-tolyl)-4-[1-(trifluoromethyl)cyclopropanecarbonyl]piperazine-1-carboxylate (591 mg, 1.39 mmol) was dissolved in DCM (3.50 mL).TFA (2.37 g, 20.79 mmol, 1.60 mL) was added dropwise to the resulting solution and stirred for 2 hr. The mixture was then basified with potassium carbonate (5 g in 10 ml H₂O), washed with DCM (2×5 ml). The organic layer was combined, washed with water, dried over Na₂SO₄ and evaporated to give [(2R,5S)-2-methyl-5-(p-tolyl)piperazin-1-yl]-[1-(trifluoromethyl)cyclopropyl]methanone (400 mg, 1.23 mmol, 88.44% yield).

LCMS(ESI): [M]⁺ m/z: calcd 326.2; found 327.2; Rt=1.023 min.

Prepared by Scheme 1.4 step 9A. Yield: 114.2 mg (36.55%).

HPLC conditions: Column: SunFire C18 100*19 mm, 5 microM; 2-10 min 40-50% MeCN; (loading pump 4 ml/min MeCN).

Compound 85: ¹H NMR (600 MHz, DMSO-d₆) δ (ppm) 0.93-1.00 (m, 1H), 1.08-1.13 (m, 3H), 1.13-1.34 (m, 6H), 2.22-2.27 (m, 3H), 2.37-2.42 (m, 2H), 2.63-3.24 (m, 1H), 3.32-4.16 (m, 2H), 4.18-5.33 (m, 2H), 5.34-5.82 (m, 3H), 7.10-7.22 (m, 4H), 7.43-7.62 (m, 1H), 8.01-8.15 (m, 1H), 9.89-10.69 (m, 1H).

LCMS(ESI): [M]⁺ m/z: calcd 517.2; found 518.2; Rt=1.094 min.

### Example 94. Compound 128 N-(6-amino-5-methylpyridin-3-yl)-2-((2S,5R)-5-methyl-2-(p-tolyl)-4-(1-(trifluoromethyl)cyclopropanecarbonyl)piperazin-1-yl)-2-oxoacetamide

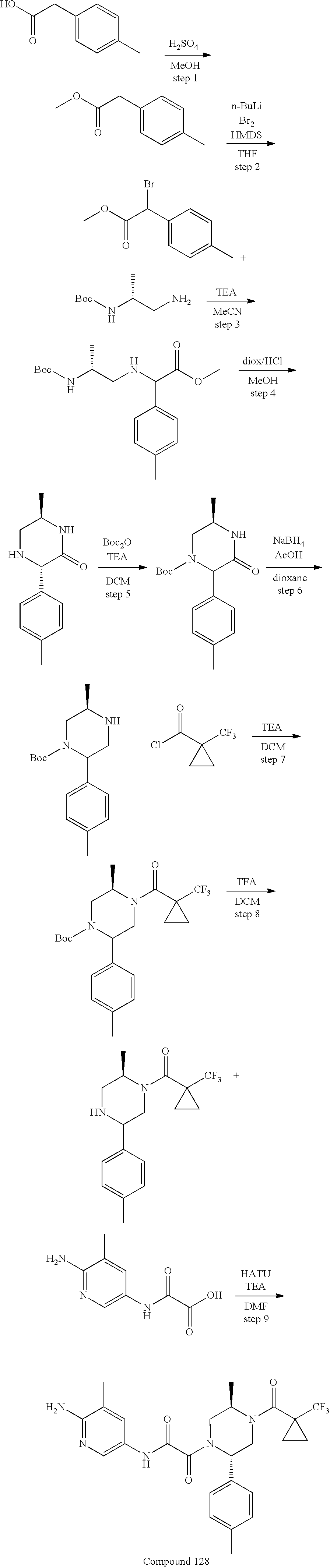

### Step 1: Synthesis of methyl 2-(p-tolyl)acetate

A 500-mL round bottom flask, with stirrer bar, was charged with 2-(p-tolyl)acetic acid (25 g, 166.47 mmol) and MeOH (250 mL). Concentrated sulfuric acid (0.1 mL) was added at rt. The resulting solution was stirred at 65° C. for 18 hr. After this time, the mixture was concentrated in vacuum, and the residue was diluted with ethyl acetate (100 mL) and saturated sodium bicarbonate (50 mL). The organic layer was washed with saturated sodium chloride (2×20 mL), dried over sodium sulfate, filtered, and concentrated under reduced pressure to afford methyl 2-(p-tolyl)acetate (26.7 g, 162.61 mmol, 97.68% yield).

¹H NMR (500 MHz, DMSO-d₆) δ (ppm) 2.22 (s, 3H), 3.36 (s, 3H), 3.62 (s, 2H), 7.12 (m, 4H).

### Step 2: Synthesis of methyl 2-bromo-2-(p-tolyl)acetate

In a nitrogen gas atmosphere, a n-butyllithium (6.90 g, 107.88 mmol, 43.16 mL) was dropwise added to a dehydrated tetrahydrofuran (100 mL) solution of hexamethyldisilazane (18.22 g, 113.01 mmol, 23.55 mL) at about −30° C. over a period of 10 minutes, followed by stirring at about −40° C. for 30 minutes. Then, to the reaction solution, a dehydrated tetrahydrofuran (20 mL) solution of methyl 2-(p-tolyl)acetate (16.87 g, 102.74 mmol) was dropwise added over a period of 20 minutes. This reaction solution was dropwise added to a dehydrated tetrahydrofuran (30 mL) solution of bromine (17.24 g, 107.88 mmol, 5.40 mL) in a nitrogen gas atmosphere at about −35° C. over a period of 1 hr. After stirring at about −35° C. for 1 hr, the temperature was raised to 0° C., and a mixed liquid of water-sodium thiosulfate aqueous solution (1:1, (v/v)) was added to the reaction solution, and extracted with ethyl acetate. Then, the extract was washed with a saturated ammonium chloride aqueous solution and a saturated salt solution and dried over anhydrous magnesium sulfate. The drying agent was removed by filtration, and the solvent was distilled off to give methyl 2-bromo-2-(p-tolyl)acetate (22.67 g, 93.26 mmol, 90.77% yield).

¹H NMR (400 MHz, CDCl₃) δ (ppm) 2.36 (s, 3H), 3.76 (s, 3H), 5.35 (s, 1H), 7.17 (d, 2H), 7.68 (d, 2H).

### Step 3: Synthesis of methyl 2-(((R)-2-((tert-butoxycarbonyl)amino)propyl)amino)-2-(p-tolyl)acetate

Prepared by Scheme 1.4 step 3. Yield: 28 g (98.98%).

LCMS(ESI): [M]⁺ m/z: calcd 336.2; found 337.2; Rt=0.946 min.

### Step 4: Synthesis of (3S,6R)-6-methyl-3-(p-tolyl)piperazin-2-one

Prepared by Scheme 1.4 step 4. Yield: 3.1 g (18.23%).

LCMS(ESI): [M]⁺ m/z: calcd 204.2; found 205.2; Rt=0.261 min.

### Step 5: Synthesis of (5R)-tert-butyl 5-methyl-3-oxo-2-(p-tolyl)piperazine-1-carboxylate

Prepared by Scheme 1.4 step 5. Yield: 4.71 g (98.31%).

LCMS(ESI): [M-t-Bu]⁺ m/z: calcd 248.2; found 249.2; Rt=1.212 min.

### Step 6: Synthesis of (5R)-tert-butyl 5-methyl-2-(p-tolyl)piperazine-1-carboxylate

Prepared by Scheme 1.4 step 6. Yield: 1.5 g (33.39%).

LCMS(ESI): [M-Boc]⁺ m/z: calcd 190.2; found 191.2; Rt=0.980 min.

### Step 7: Synthesis of (5R)-tert-butyl 5-methyl-2-(p-tolyl)-4-(1-(trifluoromethyl)cyclopropanecarbonyl)piperazine-1-carboxylate

Prepared by Scheme 1.4 step 7A. Yield: 0.591 g (81.63%).

LCMS(ESI): [M-t-Bu]⁺ m/z: calcd 370.2; found 371.2; Rt=1.605 min.

### Step 8: Synthesis of ((2R)-2-methyl-5-(p-tolyl)piperazin-1-yl)(1-(trifluoromethyl)cyclopropyl)methanone

tert-Butyl (2S,5R)-5-methyl-2-(p-tolyl)-4-[1-(trifluoromethyl)cyclopropanecarbonyl]piperazine-1-carboxylate (591 mg, 1.39 mmol) was dissolved in DCM (3.50 mL).TFA (2.37 g, 20.79 mmol, 1.60 mL) was added dropwise to the resulting solution and stirred for 2 hr. The mixture was then basified with potassium carbonate (5 g in 10 ml H₂O), washed with DCM (2×5 ml). The organic layer was combined, washed with water, dried over Na₂SO₄ and evaporated to give [(2R,5S)-2-methyl-5-(p-tolyl)piperazin-1-yl]-[1-(trifluoromethyl)cyclopropyl]methanone (400 mg, 1.23 mmol, 88.44% yield).

LCMS(ESI): [M]⁺ m/z: calcd 326.2; found 327.2; Rt=1.023 min.

### Step 9: Synthesis of N-(6-amino-5-methylpyridin-3-yl)-2-((2S,5R)-5-methyl-2-(p-tolyl)-4-(1-(trifluoromethyl)cyclopropanecarbonyl)piperazin-1-yl)-2-oxoacetamide

Prepared by Scheme 1.4 step 9A. Yield: 94.2 mg (32.65%).

HPLC conditions: Column: SunFire C18 100*19 mm, 5 microM; 2-10 min 0-80% MeCN/water; (loading pump 4 ml/min MeCN).

Compound 128: ¹H NMR (600 MHz, DMSO-d₆)₆ (ppm) 6 0.92-1.02 (m, 1H), 1.11-1.31 (m, 6H), 1.98-2.04 (m, 3H), 2.24-2.28 (m, 3H), 2.64-3.23 (m, 1H), 3.65-4.95 (m, 3H), 5.32-5.76 (m, 3H), 7.03-7.28 (m, 5H), 7.47-7.55 (m, 1H), 7.99-8.08 (m, 1H), 10.57 (s, 1H).

LCMS(ESI): [M]⁺ m/z: calcd 503.2; found 504.2; Rt=1.087 min.

### Example 95. Compound 62 N-(6-amino-5-methylpyridin-3-yl)-2-((2S,5R)-2-(3,5-dichlorophenyl)-5-methyl-4-(1-(trifluoromethyl)cyclopropanecarbonyl)piperazin-1-yl)-2-oxoacetamide

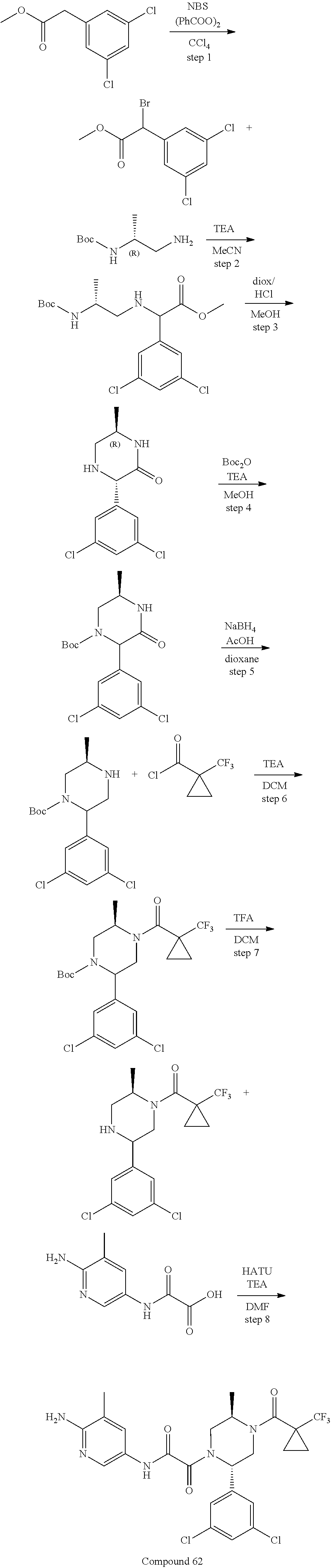

### Step 1: Synthesis of methyl 2-bromo-2-(3,5-dichlorophenyl)acetate

Prepared by Scheme 1.4, step 2. Yield: 5.05 g of crude.

¹H NMR (400 MHz, CDCl₃) δ (ppm) 3.79 (s, 3H), 5.21 (s, 1H), 7.32 (s, 1H), 7.42 (s, 2H).

### Step 2: Synthesis of methyl 2-(((R)-2-((tert-butoxycarbonyl)amino)propyl)amino)-2-(3,5-dichlorophenyl)acetate

Prepared by Scheme 1.4, step 3. Yield: 5.6 g of crude.

LCMS(ESI): [M]⁺ m/z: calcd 391.2; found 392.2; Rt=1.412 min.

### Step 3: Synthesis of (3S,6R)-3-(3,5-dichlorophenyl)-6-methylpiperazin-2-one

Prepared by Scheme 1.4, step 4. Yield: 0.2 g (5.39%).

LCMS(ESI): [M]⁺ m/z: calcd 259.2; found 260.2; Rt=0.661 min.

### Step 4: Synthesis of (5R)-tert-butyl 2-(3,5-dichlorophenyl)-5-methyl-3-oxopiperazine-1-carboxylate

Prepared by Scheme 1.4, step 5. Yield: 0.34 g (98.1%).

LCMS(ESI): [M-t-Bu]⁺ m/z: calcd 303.2; found 304.2; Rt=1.376 min.

### Step 5: Synthesis of (5R)-tert-butyl 2-(3,5-dichlorophenyl)-5-methylpiperazine-1-carboxylate

Prepared by Scheme 1.4, step 6. Yield: 62 mg of crude.

HPLC conditions: Column: YMC Triart C18 100*20 mm, 5 microM; 0-5 min 50-80% MeCN/water+0.1% NH₄OH 30 ml/min; (loading pump 4 ml/min MeOH).

LCMS(ESI): [M-Boc]⁺ m/z: calcd 245.2; found 246.2; Rt=2.512 min.

### Step 6: Synthesis of (5R)-tert-butyl 2-(3,5-dichlorophenyl)-5-methyl-4-(1-(trifluoromethyl)cyclopropanecarbonyl)piperazine-1-carboxylate

Prepared by Scheme 1.4, step 7A. Yield: 80 mg (92.56%).

LCMS(ESI): [M-t-Bu]⁺ m/z: calcd 425.2; found 426.2; Rt=1.612 min.

### Step 7: Synthesis of ((2R)-5-(3,5-dichlorophenyl)-2-methylpiperazin-1-yl)(1-(trifluoromethyl)cyclopropyl)methanone

Prepared by Scheme 1.4, step 8. Yield: 60 mg of crude.

LCMS(ESI): [M]⁺ m/z: calcd 381.2; found 382.2; Rt=1.010 min.

### Step 8: Synthesis of N-(6-amino-5-methylpyridin-3-yl)-2-((2S,5R)-2-(3,5-dichlorophenyl)-5-methyl-4-(1-(trifluoromethyl)cyclopropanecarbonyl)piperazin-1-yl)-2-oxoacetamide (Compound 62)

Prepared by Scheme 1.4, step 9A. Yield: 22.3 mg (25.37%).

HPLC conditions: Column: YMC Triart C18 100*20 mm, 5 microM; 0-5 min 55-75% MeOH/water+0.1% NH₄OH 30 ml/min; (loading pump 4 ml/min MeOH).

Compound 62: ¹H NMR (600 MHz, DMSO-d₆) δ (ppm) 1.03-1.34 (m, 8H), 1.98-2.05 (m, 3H), 3.03-3.05 (m, 1H), 3.72-4.52 (m, 3H), 4.88-5.78 (m, 3H), 7.23-7.57 (m, 4H), 8.02-8.04 (m, 1H), 10.67-10.74 (m, 1H).

LCMS(ESI): [M]⁺ m/z: calcd 558.2; found 559.2; Rt=3.250 min.

### Example 96. Compound 94 N-(6-amino-5-cyclopropylpyridin-3-yl)-2-((2S,5R)-5-methyl-4-(1-methylcyclopropanecarbonyl)-2-phenylpiperazin-1-yl)-2-oxoacetamide

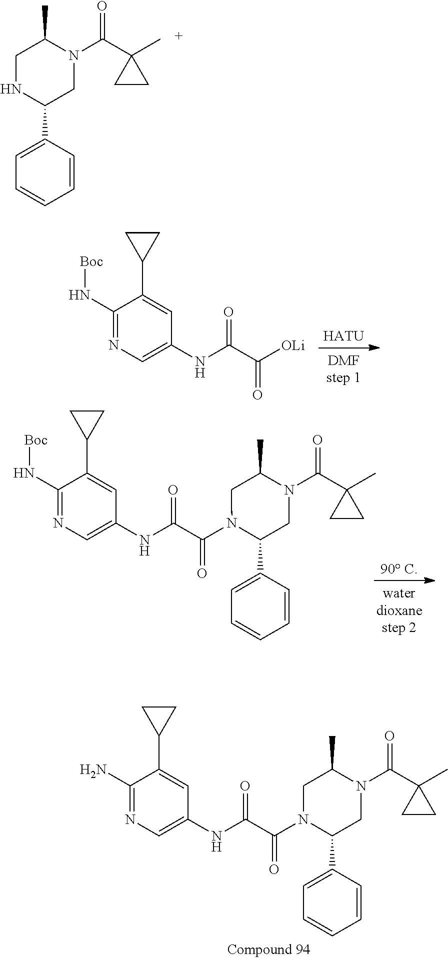

### Step 1: Synthesis of tert-butyl (3-cyclopropyl-5-(2-((2S,5R)-5-methyl-4-(1-methylcyclopropanecarbonyl)-2-phenylpiperazin-1-yl)-2-oxoacetamido)pyridin-2-yl)carbamate (

The synthesis of ((2R,5S)-2-methyl-5-phenylpiperazin-1-yl)(1-methylcyclopropyl)methanone is prepared by general procedure scheme 1.4 step 8. Yield: 0.3 g (92.5%). LCMS(ESI): [M]⁺ m/z: calcd 258.2; found 259.2; Rt=0.820 min.

Prepared by Scheme 1.4, Step 9A. Yield: 0.21 g of crude.

LCMS(ESI): [M]⁺ m/z: calcd 561.2; found 562.2; Rt=1.291 min.

### Step 2: Synthesis of N-(6-amino-5-cyclopropylpyridin-3-yl)-2-((2S,5R)-5-methyl-4-(1-methylcyclopropanecarbonyl)-2-phenylpiperazin-1-yl)-2-oxoacetamide (Compound 94)

Prepared by Boc Group Deprotection Method A. Yield: 113 mg (65.48%).

HPLC conditions: Column: YMC Triart C18 100*20 mm, 5 microM; 0-5 min 30-80% MeOH/water+0.1% NH₄OH 30 ml/min; (loading pump 4 ml/min MeOH).

Compound 94: ¹H NMR (600 MHz, DMSO-d₆) δ (ppm) 0.38-0.53 (m, 4H), 0.56-0.84 (m, 2H), 0.85-0.91 (m, 2H), 1.04 (d, 3H), 1.10-1.33 (m, 3H), 1.61-1.72 (m, 1H), 2.90-3.30 (m, 2H), 3.68-4.10 (m, 1H), 4.22-4.99 (m, 2H), 5.32-5.74 (m, 1H), 5.74-5.83 (m, 2H), 7.22-7.29 (m, 2H), 7.31-7.40 (m, 4H), 8.00-8.15 (m, 1H), 10.49 (br s, 1H).

LCMS(ESI): [M]⁺ m/z: calcd 461.2; found 462.2; Rt=2.599 min.

### Example 97. Compound 90 N-(6-amino-5-methylpyridin-3-yl)-2-((2S,5R)-2-(3-chlorophenyl)-4-isobutyryl-5-methylpiperazin-1-yl)-2-oxoacetamide

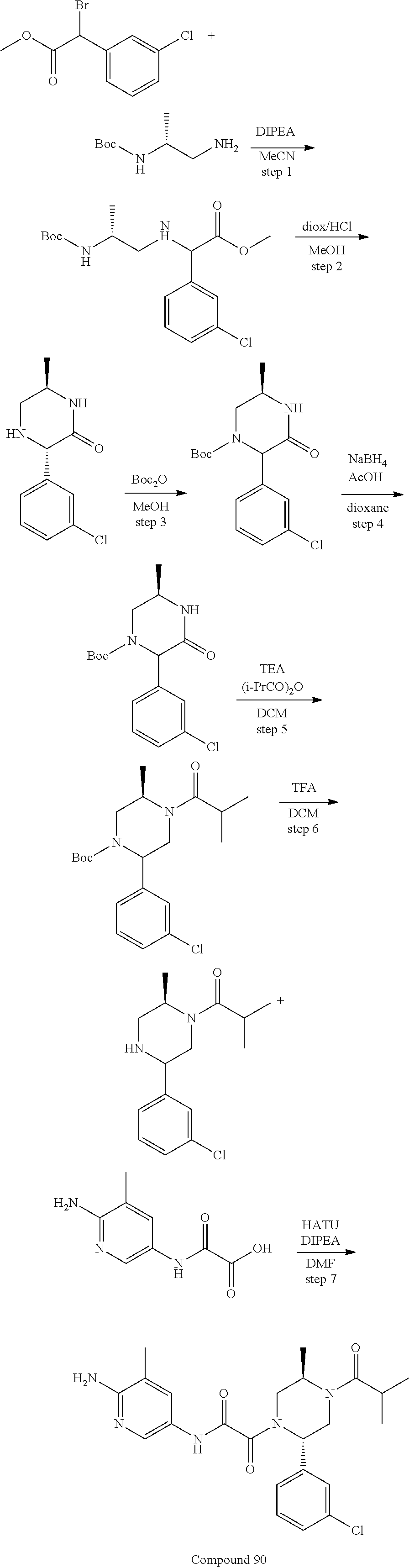

### Step 1: Synthesis of methyl 2-(((R)-2-((tert-butoxycarbonyl)amino)propyl)amino)-2-(3-chlorophenyl)acetate

Prepared by Scheme 1.4, step 3. Yield: 7.7 g of crude.

LCMS(ESI): [M]⁺ m/z: calcd 356.2; found 357.2; Rt=1.107 min.

### Step 2: Synthesis of (3S,6R)-3-(3-chlorophenyl)-6-methylpiperazin-2-one

Prepared by Scheme 1.4, step 4. Yield: 1.4 g (28.88%).

LCMS(ESI): [M]⁺ m/z: calcd 224.2; found 225.2; Rt=0.533 min.

### Step 3: Synthesis of (5R)-tert-butyl 2-(3-chlorophenyl)-5-methyl-3-oxopiperazine-1-carboxylate

Prepared by Scheme 1.4, step 5. Yield: 1.66 g (96.25%).

LCMS(ESI): [M-t-Bu]⁺ m/z: calcd 268.2; found 269.2; Rt=1.304 min.

### Step 4: Synthesis of (5R)-tert-butyl 2-(3-chlorophenyl)-5-methylpiperazine-1-carboxylate

Prepared by Scheme 1.4, step 6. Yield: 1.1 g (57.47%).

CC conditions: The crude product was purified by silica gel with EtOAc/CHCl₃ (1:1) as an eluent mixture.

LCMS(ESI): [M-Boc]⁺ m/z: calcd 210.2; found 211.2; Rt=0.890 min.

### Step 5: Synthesis of (5R)-tert-butyl 2-(3-chlorophenyl)-4-isobutyryl-5-methylpiperazine-1-carboxylate

Prepared by Scheme 1.4, step 7A. Yield: 0.6 g (97.92%).

LCMS(ESI): [M-t-Bu]⁺ m/z: calcd 324.2; found 325.2; Rt=1.449 min.

### Step 6: Synthesis of 1-((2R)-5-(3-chlorophenyl)-2-methylpiperazin-1-yl)-2-methylpropan-1-one

tert-Butyl (2S,5R)-2-(3-chlorophenyl)-5-methyl-4-(2-methylpropanoyl)piperazine-1-carboxylate (0.6 g, 1.58 mmol) was dissolved in CHCl₃ (15 mL) and TFA (3.70 g, 32.45 mmol, 2.5 mL) was added thereto. The resulting mixture was stirred at 25° C. for 16 hr. After completion the reaction mixture was evaporated, the crude product was poured into aq·K₂CO₃ solution (4 g in 10 ml of water) and the resulting mixture was extracted with CHCl₃ (2*10 ml).

Combined organic layers were dried over Na₂SO₄, filtered and evaporated. The reaction was successful. The desired product 1-[(2R,5S)-5-(3-chlorophenyl)-2-methyl-piperazin-1-yl]-2-methyl-propan-1-one (0.44 g, 1.57 mmol, 99.48% yield) was isolated.

LCMS(ESI): [M]⁺ m/z: calcd 280.2; found 281.2; Rt=0.698 min.

### Step 7: Synthesis of N-(6-amino-5-methylpyridin-3-yl)-2-((2S,5R)-2-(3-chlorophenyl)-4-isobutyryl-5-methylpiperazin-1-yl)-2-oxoacetamide (Compound 90)

Prepared by Scheme 1.4, step 9B. Yield: 39 mg (23.91%).

HPLC conditions: Column: Chromatorex C18 100*19 mm, 5 microM; 0-5 min 10-35% MeCN/water+0.1% FA 30 ml/min; (loading pump 4 ml/min MeCN).

Compound 90: ¹H NMR (600 MHz, DMSO-d₆) δ (ppm) 0.82 (m, 3H), 1.07 (m, 6H), 2.02 (m, 3H), 2.98 (m, 2H), 3.85 (m, 2H), 4.24 (m, 1H), 4.92 (m, 1H), 5.58 (m, 3H), 7.32 (m, 4H), 7.51 (m, 1H), 7.93 (m, 1H), 10.55 (m, 1H).

LCMS(ESI): [M]⁺ m/z: calcd 457.2; found 458.2; Rt=1.872 min.

### Example 98. Compound 129 (2R,5S)-tert-butyl 4-(2-((1H-pyrazolo[4,3-c]pyridin-7-yl)amino)-2-oxoacetyl)-5-(4-fluorophenyl)-2-methylpiperazine-1-carboxylate

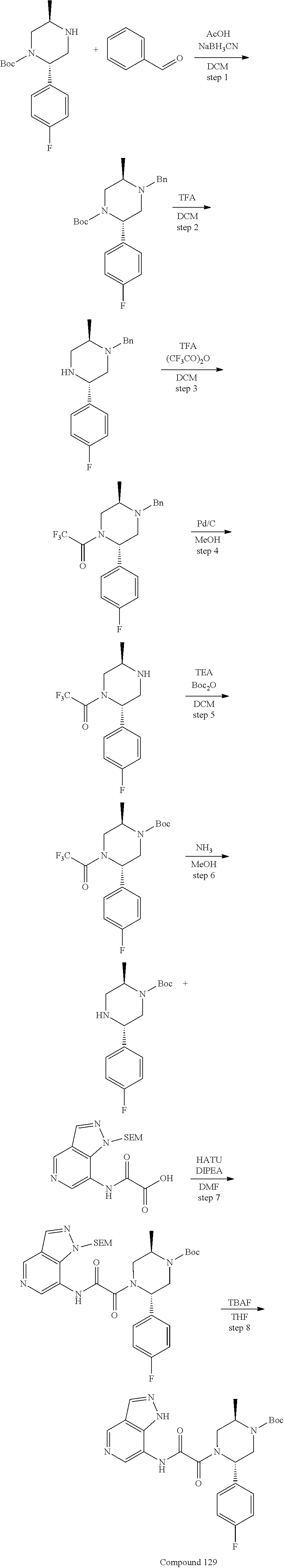

(2S,5R)-tert-butyl 2-(4-fluorophenyl)-5-methylpiperazine-1-carboxylate was prepared by Scheme 1.4, step 6. Yield: 1.3 g (27.24%).CC conditions: The crude product was purified by silica gel with MTBE/MeOH (0˜100%) as an eluent mixture.)

### Step 1: Synthesis of (2S,5R)-tert-butyl 4-benzyl-2-(4-fluorophenyl)-5-methylpiperazine-1-carboxylate

tert-Butyl (2S,5R)-2-(4-fluorophenyl)-5-methyl-piperazine-1-carboxylate (1.88 g, 6.38 mmol) was dissolved in DCM (28.66 mL) and acetic acid (382.91 mg, 6.38 mmol, 365.02 μL) was added thereto followed by addition of benzaldehyde (1.02 g, 9.56 mmol, 975.98 L). Sodium cyan borohydride (801.39 mg, 12.75 mmol) was added to the previous mixture and the resulting mixture was stirred overnight. NaHCO₃ aq. solution (15 ml) was added to the reaction mixture and the resulting mixture was extracted with DCM (2*20 ml). Combined organic layers were dried over Na₂SO₄, filtered and evaporated to obtain tert-butyl (2S,5R)-4-benzyl-2-(4-fluorophenyl)-5-methyl-piperazine-1-carboxylate (700 mg, 1.82 mmol, 28.55% yield).

LCMS(ESI): [M]⁺ m/z: calcd 384.2; found 385.2; Rt=1.271 min.

### Step 2: Synthesis of (2R,5S)-1-benzyl-5-(4-fluorophenyl)-2-methylpiperazine

tert-Butyl (2S,5R)-4-benzyl-2-(4-fluorophenyl)-5-methyl-piperazine-1-carboxylate (700 mg, 1.82 mmol) was dissolved in DCM (4 mL) and TFA (4 mL) was added thereto. The resulting mixture was stirred for 1 hr. The reaction mixture was poured into K₂CO₃ aq. solution (10 g in 45 ml of water) and the resulting mixture was extracted with DCM (2*45 ml). Combined organic layers were dried over Na₂SO₄, filtered and concentrated in vacuum to obtain (2R,5S)-1-benzyl-5-(4-fluorophenyl)-2-methyl-piperazine (450 mg, 1.58 mmol, 86.92% yield).

### Step 3: Synthesis of 1-((2S,5R)-4-benzyl-2-(4-fluorophenyl)-5-methylpiperazin-1-yl)-2,2,2-trifluoroethanone

(2R,5S)-1-Benzyl-5-(4-fluorophenyl)-2-methyl-piperazine (0.4 g, 1.41 mmol) and TEA (427.01 mg, 4.22 mmol, 588.16 L) were mixed together in DCM (15 mL) and the resulting mixture was cooled to −5° C. in an ice/MeOH bath. (2,2,2-Trifluoroacetyl) 2,2,2-trifluoroacetate (354.52 mg, 1.69 mmol, 238.41 L) was added dropwise to the previous solution and the resulting mixture was allowed to warm to room temperature and stirred overnight. The reaction mixture was diluted with DCM (10 ml) and the resulting mixture was washed with water (2*5 ml), dried over Na₂SO₄, filtered and evaporated to obtain 2,2,2-trifluoro-1-[(2S,5R)-4-benzyl-2-(4-fluorophenyl)-5-methyl-piperazin-1-yl]ethanone (443 mg, 1.16 mmol, 82.80% yield).

LCMS(ESI): [M]⁺ m/z: calcd 380.2; found 381.2; Rt=1.606 min.

### Step 4: Synthesis of 2,2,2-trifluoro-1-((2S,5R)-2-(4-fluorophenyl)-5-methylpiperazin-1-yl)ethanone

The mixture of 2,2,2-trifluoro-1-[(2S,5R)-4-benzyl-2-(4-fluorophenyl)-5-methyl-piperazin-1-yl]ethanone (443 mg, 1.16 mmol) and palladium, 10% on carbon, Type 487, dry (123.94 mg, 1.16 mmol) in MeOH (5 mL) was subjected to hydrogenation (latm) at 20° C. for 36 hr. Then the mixture was filtered and evaporated to dryness affording 2,2,2-trifluoro-1-[(2S,5R)-2-(4-fluorophenyl)-5-methyl-piperazin-1-yl]ethanone (0.26 g, crude).

LCMS(ESI): [M]⁺ m/z: calcd 290.2; found 291.2; Rt=0.477 min.

### Step 5: Synthesis of (2R,5S)-tert-butyl 5-(4-fluorophenyl)-2-methyl-4-(2,2,2-trifluoroacetyl)piperazine-1-carboxylate

2,2,2-Trifluoro-1-[(2S,5R)-2-(4-fluorophenyl)-5-methyl-piperazin-1-yl]ethanone (0.26 g, 895.76 μmol) and TEA (135.96 mg, 1.34 mmol, 187.28 L) was dissolved in DCM (10 30 mL),then di-tert-butyl dicarbonate (215.05 mg, 985.34 mol, 226.13 L) was added dropwise under ice/water cooling, after that the reaction mixture was stirred at 20° C. for 16 hr. After the reaction mixture was washed with NaHSO₄ aq. three times, the DCM layer was dried over Na₂SO₄, filtered and concentrated on vacuum to obtained tert-butyl (2R,5S)-5-(4-fluorophenyl)-2-methyl-4-(2,2,2-trifluoroacetyl)piperazine-1-carboxylate (355 mg, crude).

LCMS(ESI): [M-Boc]⁺ m/z: calcd 290.2; found 291.2; Rt=1.693 min.

### Step 6: Synthesis of (2R,5S)-tert-butyl 5-(4-fluorophenyl)-2-methylpiperazine-1-carboxylate

tert-Butyl (2R,5S)-5-(4-fluorophenyl)-2-methyl-4-(2,2,2-trifluoroacetyl)piperazine-1-carboxylate (355 mg, 909.39 mol) was dissolved in NH₃/MeOH (10 mL) and the resulting mixture was stirred overnight. The reaction mixture was concentrated in vacuum to obtain tert-butyl (2R,5S)-5-(4-fluorophenyl)-2-methyl-piperazine-1-carboxylate (220 mg, 747.38 μmol, 82.18% yield).

LCMS(ESI): [M-]⁺ m/z: calcd 294.2; found 295.2; Rt=1.064 min.

### Step 7: Synthesis of (2R,5S)-tert-butyl 5-(4-fluorophenyl)-2-methyl-4-(2-oxo-2-((1-((2-(trimethylsilyl)ethoxy)methyl)-1H-pyrazolo[4,3-c]pyridin-7-yl)amino)acetyl)piperazine-1-carboxylate

tert-Butyl (2R,5S)-5-(4-fluorophenyl)-2-methyl-piperazine-1-carboxylate (0.104 g, 353.30 μmol), 2-oxo-2-[[1-(2-trimethylsilylethoxymethyl)pyrazolo[4,3-c]pyridin-7-yl]amino]acetic acid (118.86 mg, 353.30 μmol) and DIPEA (136.99 mg, 1.06 mmol, 184.62 μL) was dissolved in the DMF (5 mL) and the resulting mixture was stirred 5 min. HATU (174.64 mg, 459.30 μmol) was added thereto and the reaction mixture was stirred at 25° C. for 12 hr. After completion the reaction mixture was diluted with EtOAc (50 ml) and washed with aqueous NaCl (4*25 ml), dried over Na₂SO₄ and evaporated under reduced pressure to afford crude tert-butyl (2R,5S)-5-(4-fluorophenyl)-2-methyl-4-[2-oxo-2-[[1-(2-trimethylsilylethoxymethyl)pyrazolo[4,3-c]pyridin-7-yl]amino]acetyl]piperazine-1-carboxylate (0.23 g, 375.35 mol, 106.24% yield).

LCMS(ESI): [M]⁺ m/z: calcd 612.2; found 613.2; Rt=1.685 min.

### Step 8: Synthesis of (2R,5S)-tert-butyl 4-(2-((1H-pyrazolo[4,3-c]pyridin-7-yl)amino)-2-oxoacetyl)-5-(4-fluorophenyl)-2-methylpiperazine-1-carboxylate (Compound 129)

Before experiment the solvent of TBAF (213.35 mg, 815.97 mol, 236.26 L) in the THF(1M) and the solvent THF (5 mL) was previously dried over the molecular sieves. tert-Butyl (2R,5S)-5-(4-fluorophenyl)-2-methyl-4-[2-oxo-2-[[1-(2-trimethylsilylethoxymethyl)pyrazolo[4,3-c]pyridin-7-yl]amino]acetyl]piperazine-1-carboxylate (0.1 g, 163.19 mol) was dissolved in the THF (5 mL) under argon stream and TBAF (213.35 mg, 815.97 μmol, 236.26 L) (solution in the THF (1M)) was added thereto. The resulting mixture was refluxed at 75° C. for 72 hr. After the completion the solvent was evaporated under reduced pressure and the residue was diluted by EtOAc (10 mL.) and washed by H₂O (3*2 ml) and dried over Na₂SO₄. The solution was evaporated in vacuum to get crude product (0.07g) that was purified twice. The conditions of the first HPLC: device (Mobile Phase, Column): SYSTEM 20-20-45% 0-1-6 min H₂O/MeCN/0.1% FA, flow: 30 ml/min (loading pump 4 ml/min MeCN); column: Chromatorex 18 SMB100-5T 100×19 mm 5 μm. The obtained 0.0104 g. of crude (92%) was purified with second reverse phase HPLC in the following conditions: device (Mobile Phase, Column): SYSTEM 30-30-80% 0-1-6 min H₂O/MeOH/0.1% NH₄OH, flow: 30 ml/min (loading pump 4 ml/min MeOH); column: YMC Triart C18 100×20 mm, 5 um. Pure product tert-butyl (2R,5S)-5-(4-fluorophenyl)-2-methyl-4-[2-oxo-2-(1H-pyrazolo[4,3-c]pyridin-7-ylamino)acetyl]piperazine-1-carboxylate (4.20 mg, 8.70 mol, 5.33% yield) was obtained.

Compound 129: ¹H NMR (600 MHz, DMSO-d₆) δ (ppm) 1.16-1.23 (m, 3H), 1.32-1.40 (m, 9H), 2.84-3.07 (m, 1H), 3.49-3.60 (m, 1H), 3.87-4.06 (m, 1H), 4.07-4.29 (m, 1H), 4.30-4.47 (m, 1H), 5.46-5.64 (m, 1H), 7.15-7.28 (m, 2H), 7.34-7.47 (m, 2H), 8.29-8.36 (m, 1H), 8.41-8.50 (m, 1H), 8.90-8.99 (m, 1H), 11.27 (br s, 1H), 13.18 (br s, 1H).

LCMS(ESI): [M]⁺ m/z: calcd 482.2; found 483.2; Rt=2.993 min.

### Example 99. Compound 87 N-(6-amino-5-ethylpyridin-3-yl)-2-((2S,5R)-2-(3-chloro-4-fluorophenyl)-4-isobutyryl-5-methylpiperazin-1-yl)-2-oxoacetamide

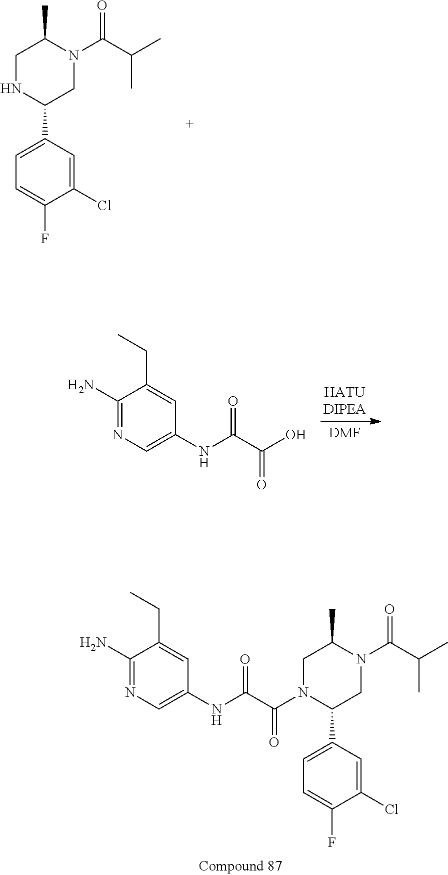

The synthesis of 1-((2R)-5-(3-chloro-4-fluorophenyl)-2-methylpiperazin-1-yl)-2-methylpropan-1-one is given in Compound 74 is given by tert-Butyl (2S,5R)-2-(3-chloro-4-fluoro-phenyl)-5-methyl-4-(2-methylpropanoyl)piperazine-1-carboxylate (0.27 g, 676.86 mol) was dissolved in DCM (10 mL) and TFA (385.89 mg, 3.38 mmol, 260.74 L) was added to reaction mixture at 0° C. then the solution was allowed to warm to 25° C. and stirred for 16 hr. Reaction mixture was diluted with DCM (30 ml) and washed by saturated aqueous NaHCO₃ (3*30 ml). The resulting organic phase was dried over Na₂SO₄ and evaporated under reduced pressure to afford 1-[(2R,5S)-5-(3-chloro-4-fluoro-phenyl)-2-methyl-piperazin-1-yl]-2-methyl-propan-1-one (0.12 g, 401.63 mol, 59.34% yield).

Prepared by Scheme 1.4, step 9B. Yield: 53.5 mg (29.66%).

HPLC conditions: Column: Chromatorex C18 100*19 mm, 5 microM; 0-6 min 10-40% MeCN-water+0.2% FA; (loading pump 4 ml/min MeCN).

Compound 87: ¹H NMR (600 MHz, DMSO-d₆) δ (ppm) 0.70-0.78 (m, 1H), 0.87-0.91 (m, 2H), 0.92 (d, 1H), 0.97-1.03 (m, 2H), 1.08-1.17 (m, 4H), 1.24 (t, 2H), 2.62-3.26 (m, 3H), 3.39-4.91 (m, 4H), 5.31-5.69 (m, 1H), 7.23-7.54 (m, 3H), 7.63 (s, 2H), 7.83-7.90 (m, 1H), 8.15-8.38 (m, 1H), 10.82-11.25 (m, 1H), 13.10 (s, 1H).

LCMS(ESI): [M]⁺ m/z: calcd 489.2; found 490.2; Rt=3.180 min.

### Example 100. Compound 9 2-((2S,5R)-2-(3-chlorophenyl)-5-methyl-4-pivaloylpiperazin-1-yl)-2-oxo-N-(1H-pyrazolo[4,3-c]pyridin-7-yl)acetamide

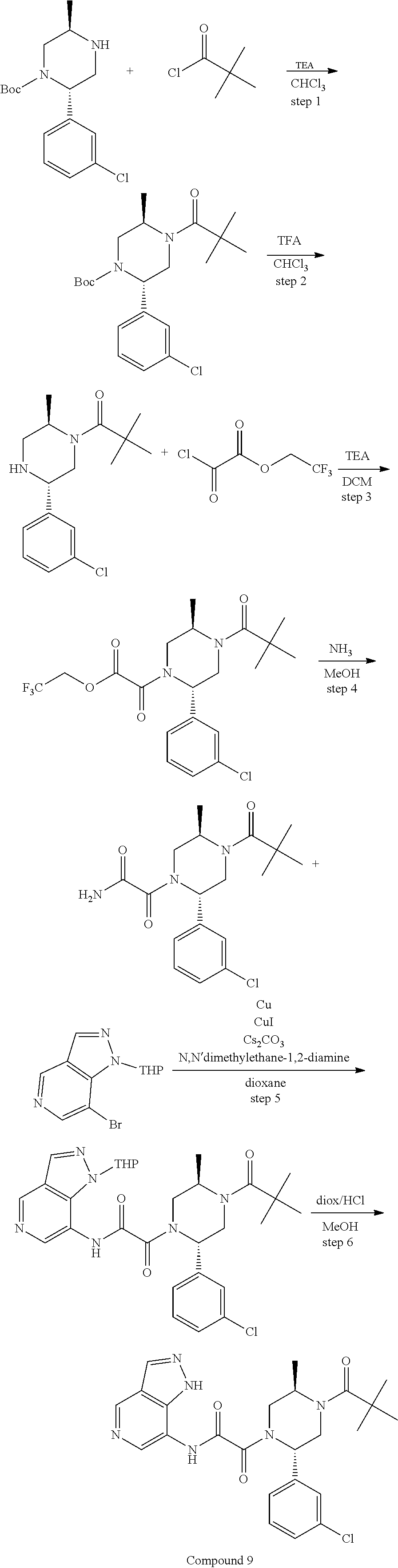

(5R)-tert-butyl 2-(3-chlorophenyl)-5-methylpiperazine-1-carboxylate is prepared by Scheme 1.4, step 6. Yield: 1.1 g (57.47%).

CC conditions: The crude product was purified by silica gel with EtOAc/CHCl₃ (1:1) as an eluent mixture.

LCMS(ESI): [M-Boc]⁺ m/z: calcd 210.2; found 211.2; Rt=0.890 min.

### Step 1: Synthesis of (2S,5R)-tert-butyl 2-(3-chlorophenyl)-5-methyl-4-pivaloylpiperazine-1-carboxylate

tert-Butyl (2S,5R)-2-(3-chlorophenyl)-5-methyl-piperazine-1-carboxylate (0.3 g, 965.19 μmol) and TEA (217.80 mg, 2.15 mmol, 0.3 mL) were mixed together in CHCl₃ (5 mL) and the resulting solution was cooled to 5° C. in an ice bath. 2,2-Dimethylpropanoyl chloride (147.75 mg, 1.23 mmol, 0.15 mL) was added dropwise to the previous solution and the resulting mixture was allowed to warm to rt and stirred overnight. After completion the reaction mixture was diluted with CHCl₃ (5 ml) and the resulting solution was washed with water (2*10 ml), dried over Na₂SO₄, filtered and evaporated. The desired product tert-butyl (2S,5R)-2-(3-chlorophenyl)-4-(2,2-dimethylpropanoyl)-5-methyl-piperazine-1-carboxylate (0.38 g, 962.19 mol, 99.69% yield) was isolated.

LCMS(ESI): [M-t-Bu]⁺ m/z: calcd 338.2; found 339.2; Rt=1.536 min.

### Step 2: Synthesis of 1-((2R,5S)-5-(3-chlorophenyl)-2-methylpiperazin-1-yl)-2,2-dimethylpropan-1-one

tert-Butyl (2S,5R)-2-(3-chlorophenyl)-4-(2,2-dimethylpropanoyl)-5-methyl-piperazine-1-carboxylate (0.38 g, 962.19 μmol) was dissolved in CHCl₃ (10 mL) and TFA (1.48 g, 12.98 mmol, 1 mL) was added thereto. The resulting mixture was stirred at 25° C. for 16 hr. After completion the reaction mixture was evaporated, the crude product was poured into aq·K₂CO₃ solution (4 g in 10 ml of water) and the resulting mixture was extracted with CHCl₃ (2*10 ml). Combined organic layers were dried over Na₂SO₄, filtered and evaporated.

The desired product 1-[(2R,5S)-5-(3-chlorophenyl)-2-methyl-piperazin-1-yl]-2,2-dimethyl-propan-1-one (0.28 g, 949.74 mol, 98.71% yield) was isolated.

LCMS(ESI): [M]⁺ m/z: calcd 294.2; found 295.2; Rt=0.945 min.

### Step 3: Synthesis of 2,2,2-trifluoroethyl 2-((2S,5R)-2-(3-chlorophenyl)-5-methyl-4-pivaloylpiperazin-1-yl)-2-oxoacetate

1-[(2R,5S)-5-(3-Chlorophenyl)-2-methyl-piperazin-1-yl]-2,2-dimethyl-propan-1-one (0.18 g, 610.54 mol) and TEA (145.20 mg, 1.43 mmol, 0.2 mL) were mixed together in DCM (10.00 mL) and the resulting solution was cooled to 5° C. in an ice bath. 2,2,2-Trifluoroethyl 2-chloro-2-oxo-acetate (0.2 g, 1.05 mmol) was added dropwise to the previous solution and the resulting mixture was allowed to warm to rt and stirred overnight. After completion the reaction mixture was diluted with CHCl₃ (5 ml) and the resulting solution was washed with water (2*10 ml), dried over Na₂SO₄, filtered and evaporated. The desired product 2,2,2-trifluoroethyl 2-[(2S,5R)-2-(3-chlorophenyl)-4-(2,2-dimethylpropanoyl)-5-methyl-piperazin-1-yl]-2-oxo-acetate (0.27 g, 601.52 mol, 98.52% yield) was isolated.

LCMS(ESI): [M]⁺ m/z: calcd 448.2; found 449.2; Rt=1.498 min.

### Step 4: Synthesis of 2-((2S,5R)-2-(3-chlorophenyl)-5-methyl-4-pivaloylpiperazin-1-yl)-2-oxoacetamide

To the solution of 2,2,2-trifluoroethyl 2-[(2S,5R)-2-(3-chlorophenyl)-4-(2,2-dimethylpropanoyl)-5-methyl-piperazin-1-yl]-2-oxo-acetate (0.27 g, 601.52 mol) in MeOH (5 mL) was added MeOH/NH₃ (5 mL). The resulting reaction mixture was stirred at room temperature for 3 hr. After 3 hr, the reaction mixture was evaporated under reduced pressure. The desired product 2-[(2S,5R)-2-(3-chlorophenyl)-4-(2,2-dimethylpropanoyl)-5-methyl-piperazin-1-yl]-2-oxo-acetamide (0.22 g, 601.33 mol, 99.97% yield) was isolated.

LCMS(ESI): [M]⁺ m/z: calcd 365.2; found 366.2; Rt=1.130 min.

### Step 5: Synthesis of 2-((2S,5R)-2-(3-chlorophenyl)-5-methyl-4-pivaloylpiperazin-1-yl)-2-oxo-N-(1-(tetrahydro-2H-pyran-2-yl)-1H-pyrazolo[4,3-c]pyridin-7-yl)acetamide

2-[(2S,5R)-2-(3-Chlorophenyl)-4-(2,2-dimethylpropanoyl)-5-methyl-piperazin-1-yl]-2-oxo-acetamide (0.22 g, 601.33 mol), 7-bromo-1-tetrahydropyran-2-yl-pyrazolo[4,3-c]pyridine (0.15 g, 531.66 mol), copper (0.04 g, 629.43 mol), copper (I) iodide (0.12 g, 630.09 mol, 21.35 L) and cesium carbonate (0.4 g, 1.23 mmol) were mixed together in dioxane (10 mL). The resulting suspension was degassed with argon at 25° C. for 0.1 hr. (¹R,²R)—N¹,N²-Dimethylcyclohexane-1,2-diamine (135.30 mg, 951.21 mol, 0.15 mL) was added thereto and the resulting mixture was stirred for 16 hr at 100° C. After completion the reaction mixture was filtered and the filtrate was concentrated in vacuum. The desired product 2-[(2S,5R)-2-(3-chlorophenyl)-4-(2,2-dimethylpropanoyl)-5-methyl-piperazin-1-yl]-2-oxo-N-(1-tetrahydropyran-2-ylpyrazolo[4,3-c]pyridin-7-yl)acetamide (0.34 g, 599.57 mol, 99.71% yield) was isolated.

LCMS(ESI): [M]⁺ m/z: calcd 567.2; found 568.2; Rt=1.120 min.

### Step 6: Synthesis of 2-((2S,5R)-2-(3-chlorophenyl)-5-methyl-4-pivaloylpiperazin-1-yl)-2-oxo-N-(1H-pyrazolo[4,3-c]pyridin-7-yl)acetamide (Compound 9)

The solution of 2-[(2S,5R)-2-(3-chlorophenyl)-4-(2,2-dimethylpropanoyl)-5-methyl-piperazin-1-yl]-2-oxo-N-(1-tetrahydropyran-2-ylpyrazolo[4,3-c]pyridin-7-yl)acetamide (0.34 g, 599.57 mol) in MeOH (10 mL) and diox/HCl (5 mL) was stirred at 25° C. for 16 hr. Upon completion, the reaction mixture was concentrated under reduced pressure to obtain crude product. The obtained crude product was purified by reverse phase HPLC chromatography (Device (Mobile Phase, Column): SYSTEM 10-10-60% 0-1-6 min H₂O/MeCN/0.1% NH₄OH, flow: 30 ml/min (loading pump 4 ml/min MeCN) target mass 482 column: XBridge BEH C18 5 μm 130A) to afford product 2-[(2S,5R)-2-(3-chlorophenyl)-4-(2,2-dimethylpropanoyl)-5-methyl-piperazin-1-yl]-2-oxo-N-(1H-pyrazolo[4,3-c]pyridin-7-yl)acetamide (14 mg, 28.99 mol, 4.83% yield).

Compound 9: LCMS(ESI): [M+1]⁺ m/z: calcd 482.2; found 483.2; Rt=2.673 min.

### Example 101. Compound 113 2-((2S,5R)-4-isobutyryl-5-methyl-2-(3-(4-methylpiperazin-1-yl)phenyl)piperazin-1-yl)-2-oxo-N-(1H-pyrazolo[4,3-c]pyridin-7-yl)acetamide

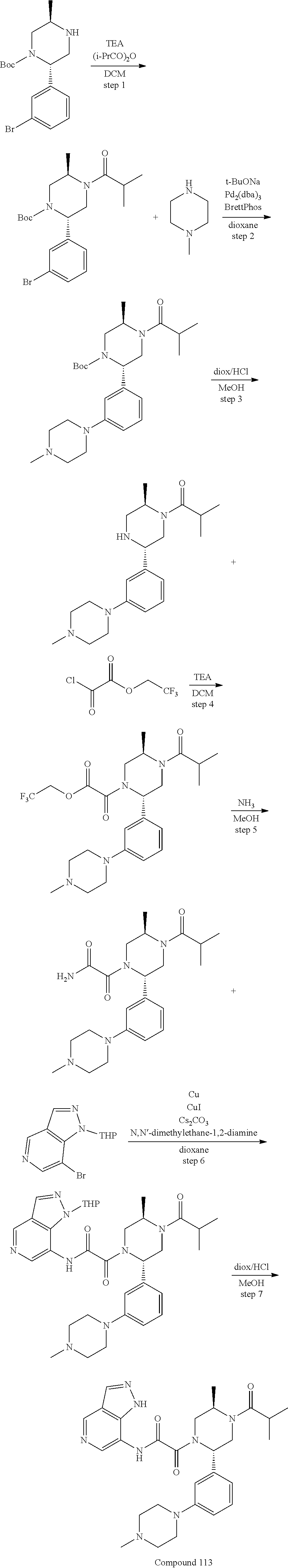

### Step 1: Synthesis of (2S,5R)-tert-butyl 2-(3-bromophenyl)-4-isobutyryl-5-methylpiperazine-1-carboxylate

Prepared by Scheme 1.4, step 7A. Yield: 0.6 g of crude.

LCMS(ESI): [M-t-Bu]⁺ m/z: calcd 369.2; found 370.2; Rt=1.456 min.

### Step 2: Synthesis of (2S,5R)-tert-butyl 4-isobutyryl-5-methyl-2-(3-(4-methylpiperazin-1-yl)phenyl)piperazine-1-carboxylate

tert-Butyl (2S,5R)-2-(3-bromophenyl)-5-methyl-4-(2-methylpropanoyl)piperazine-1-carboxylate (0.2 g, 470.19 mol), 1-methylpiperazine (56.51 mg, 564.23 mol, 62.58 L) and tris(dibenzylideneacetone)dipalladium (O) (21.53 mg, 23.51 mol) were mixed together in dioxane (14.99 mL) and sodium tert-butoxide (67.78 mg, 705.29 mol) was added thereto. The resulting mixture was evacuated and backfilled three times with argon. BrettPhos (27.21 mg, 47.02 mol) was added to the previous mixture and the resulting mixture was heated at 100° C. (oil bath) for 14 hr. Upon completion of the reaction the resulting mixture was poured into 40 ml water, stirred for 15 min and extracted with EtOAc (3*15 ml), combined organic layers were dried over Na₂SO₄, filtered and concentrated in vacuum to obtain tert-butyl (2S,5R)-5-methyl-2-[3-(4-methylpiperazin-1-yl)phenyl]-4-(2-methylpropanoyl)piperazine-1-carboxylate (0.15 g, crude).

LCMS(ESI): [M]⁺ m/z: calcd 444.2; found 445.2; Rt=0.870 min.

### Step 3: Synthesis of 2-methyl-1-((2R,5S)-2-methyl-5-(3-(4-methylpiperazin-1-yl)phenyl)piperazin-1-yl)propan-1-one

To a mixture of diox/HCl (5 mL) and MeOH (5 mL), tert-butyl (2S,5R)-5-methyl-2-[3-(4-methylpiperazin-1-yl)phenyl]-4-(2-methylpropanoyl)piperazine-1-carboxylate (0.3 g, 674.75 mol) was added in one portion. The resulting mixture was stirred at 25° C. for 16 hr. Upon completion of the reaction, solvents were evaporated to dryness, 20 ml of water was added to the residue, mixture was washed with DCM (2*15 ml), aqueous phase was basified with K₂CO₃ to alkaline pH and extracted with DCM (3*15 ml), combined organic layers were dried over Na₂SO₄, filtered and concentrated in vacuum, affording 2-methyl-1-[(2R,5S)-2-methyl-5-[3-(4-methylpiperazin-1-yl)phenyl]piperazin-1-yl]propan-1-one (0.1 g, crude).

LCMS(ESI): [M]⁺ m/z: calcd 344.2; found 345.2; Rt=0.472 min.

### Step 4: Synthesis of 2,2,2-trifluoroethyl 2-((2S,5R)-4-isobutyryl-5-methyl-2-(3-(4-methylpiperazin-1-yl)phenyl)piperazin-1-yl)-2-oxoacetate

2-Methyl-1-[(2R,5S)-2-methyl-5-[3-(4-methylpiperazin-1-yl)phenyl]piperazin-1-yl]propan-1-one (0.1 g, 290.28 mol) and TEA (44.06 mg, 435.42 mol, 60.69 L) were mixed together in DCM (10 mL) and the resulting solution was cooled to 5° C. in an ice bath. 2,2,2-Trifluoroethyl 2-chloro-2-oxo-acetate (60.83 mg, 319.31 mol) was added dropwise to the previous solution and the resulting mixture was allowed to warm to rt and stirred overnight. Upon completion of the reaction, the mixture was washed with brine (2*20 ml), organic layer was dried over Na₂SO₄, filtered and concentrated in vacuum to affording 2,2,2-trifluoroethyl 2-((2S,5R)-4-isobutyryl-5-methyl-2-(3-(4-methylpiperazin-1-yl)phenyl)piperazin-1-yl)-2-oxoacetate (0.2 g, crude).

LCMS(ESI): [M]⁺ m/z: calcd 498.2; found 499.2; Rt=0.824 min.

### Step 5: Synthesis of 2-((2S,5R)-4-isobutyryl-5-methyl-2-(3-(4-methylpiperazin-1-yl)phenyl)piperazin-1-yl)-2-oxoacetamide

2,2,2-Trifluoroethyl 2-((2S,5R)-4-isobutyryl-5-methyl-2-(3-(4-methylpiperazin-1-yl)phenyl) piperazin-1-yl)-2-oxoacetate (0.2 g) was dissolved in MeOH/NH₃ (10 mL). The resulting clear solution was left overnight at rt. Upon completion of the reaction the resulting mixture was concentrated to dryness. Crude product was subjected by HPLC (column: YMC Triart C18 100×20 mm, 5 um; mobile phase: 20-20-55% 0-1-6 min H₂O/MeCN/0.1% FA, flow rate: 30 ml/min (loading pump 4 ml/min MeCN), affording 2-oxo-2-[(2S,5R)-5-methyl-2-[3-(4-methylpiperazin-1-yl)phenyl]-4-(2-methylpropanoyl)piperazin-1-yl]acetamide (18.2 mg, 43.80 mol, 10.61% yield).

LCMS(ESI): [M]⁺ m/z: calcd 415.2; found 416.2; Rt=0.774 min.

### Step 5: Synthesis of 2-((2S,5R)-4-isobutyryl-5-methyl-2-(3-(4-methylpiperazin-1-yl)phenyl)piperazin-1-yl)-2-oxo-N-(1-(tetrahydro-2H-pyran-2-yl)-1H-pyrazolo[4,3-c]pyridin-7-yl)acetamide

2-Oxo-2-[(2S,5R)-5-methyl-2-[3-(4-methylpiperazin-1-yl)phenyl]-4-(2-methylpropanoyl)piperazin-1-yl]acetamide (0.018 g, 43.32 mol), 7-bromo-1-tetrahydropyran-2-yl-pyrazolo[4,3-c]pyridine (12.22 mg, 43.32 mol), CuI (8.25 mg, 43.32 mol, 1.47 μL), cesium carbonate (28.23 mg, 86.64 mol), Cu (2.75 mg, 43.32 mol) and (IS,2S)—N,N′-bis-methyl-1,2-cyclohexane-diamine (9.24 mg, 64.98 mol, 10.25 L) were mixed in dioxane (3.99 mL) under argon. The resulting mixture was allowed to stir at 100° C. for 16 hr in vial. Upon completion of the reaction, dioxane was evaporated and residue was subjected by HPLC (column: XBridge C18 100×19 mm, 5 um; mobile phase: 30-80% 0-5 min H₂O/MeOH/0.1% NH₄OH, flow rate: 30 ml/min (loading pump 4 ml/min MeOH), affording 2-oxo-2-[(2S,5R)-5-methyl-2-[3-(4-methylpiperazin-1-yl)phenyl]-4-(2-methylpropanoyl)piperazin-1-yl]-N-(1-tetrahydropyran-2-ylpyrazolo[4, 3-c]pyridin-7-yl)acetamide (15.10 mg, 24.48 mol, 56.52% yield).

LCMS(ESI): [M]⁺ m/z: calcd 616.2; found 617.2; Rt=1.891 min.

### Step 7: Synthesis of 2-((2S,5R)-4-isobutyryl-5-methyl-2-(3-(4-methylpiperazin-1-yl)phenyl)piperazin-1-yl)-2-oxo-N-(1H-pyrazolo[4,3-c]pyridin-7-yl)acetamide (Compound 113)

2-Oxo-2-[(2S,5R)-5-methyl-2-[3-(4-methylpiperazin-1-yl)phenyl]-4-(2-methylpropanoyl)piperazin-1-yl]-N-(1-tetrahydropyran-2-ylpyrazolo[4,3-c]pyridin-7-yl)acetamide (0.018 g, 29.19 mol) was dissolved in a mixture of MeOH (1 mL) and diox/HCl (1 mL). The resulting mixture was left to stir at 25° C. for 2 hr. After that time, solvent was evaporated to dryness. Crude product was subjected by HPLC (column: Chromatorex 18 SMB100-5T 100×19 mm, 5 um; mobile phase: 0˜30% 0-5 min H₂O/MeCN/0.1% FA, flow rate: 30 ml/min (loading pump 4 ml/min MeCN), affording 2-oxo-N-(1H-pyrazolo[4, 3-c]pyridin-7-yl)-2-[(2S,5R)-5-methyl-2-[3-(4-methylpiperazin-1-yl)phenyl]-4-(2-methylpropanoyl)piperazin-1-yl]acetamide (6.3 mg, 10.40 mol, 35.65% yield, 2HCl).

Compound 113: LCMS(ESI): [M]⁺ m/z: calcd 532.2; found 533.2; Rt=1.737 min.

### Example 102. Compound 137 N-(6-amino-5-methylpyridin-3-yl)-2-((2S,5R)-5-methyl-4-(1-(trifluoromethyl)cyclopropanecarbonyl)-2-(3-(trifluoromethyl)phenyl)piperazin-1-yl)-2-oxoacetamide

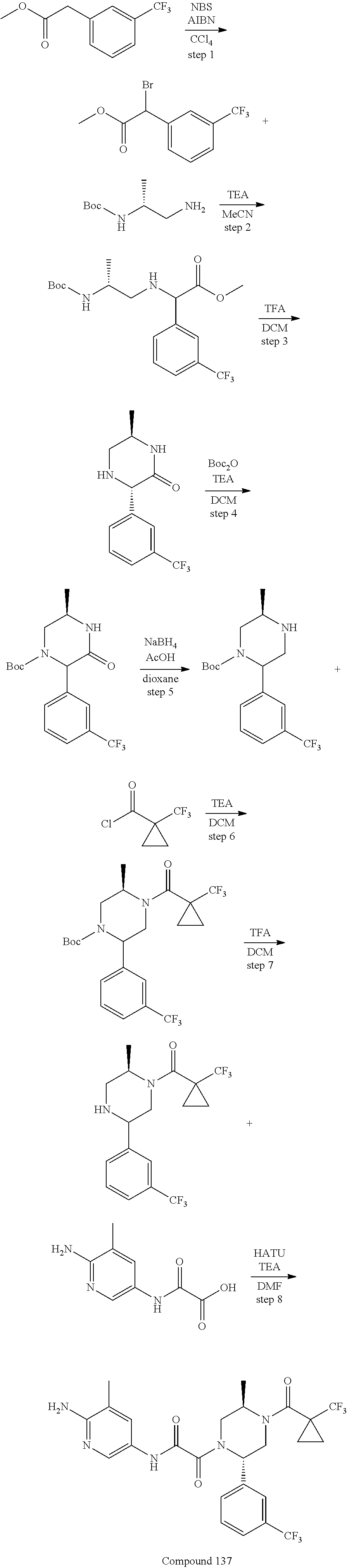

### Step 1: Synthesis of methyl 2-bromo-2-(3-(trifluoromethyl)phenyl)acetate

To a mixture of methyl 2-[3-(trifluoromethyl)phenyl]acetate (6 g, 27.50 mmol, 4.84 mL) in CCl₄ (120 mL), 2,2′-azobis(2-methylpropionitrile) (677.40 mg, 4.13 mmol) and N-bromosuccinimide (7.34 g, 41.25 mmol, 3.50 mL) was added in one portion. The resulting mixture was left to stir at 80° C. for 15 hr. Upon completion of the reaction, solvent was evaporated; 30 ml of water was added to residue. Water phase was extracted with MTBE (2*100 ml) combined organic layers was washed with K₂CO₃ water solution, dried over Na₂SO₄, filtered and concentrated under reduced pressure to obtain methyl 2-bromo-2-[3-(trifluoromethyl)phenyl]acetate (8.5 g, crude).

¹H NMR (400 MHz, CDCl₃) δ (ppm) 3.79 (s, 3H), 5.35 (s, 1H), 7.49 (t, 1H), 7.59 (d, 1H), 7.75 (m, 2H).

### Step 2: Synthesis of methyl 2-(((R)-2-((tert-butoxycarbonyl)amino)propyl)amino)-2-(3-(trifluoromethyl)phenyl)acetate

Prepared by Scheme 1.4, step 3. Yield: 6.5 g (93.32%).

LCMS(ESI): [M]⁺ m/z: calcd 390.2; found 391.2; Rt=1.276 min.

### Step 3: Synthesis of (3S,6R)-6-methyl-3-(3-(trifluoromethyl)phenyl)piperazin-2-one

TFA (15 mL) was added to a solution of methyl 2-[[(2R)-2-(tert-butoxycarbonylamino)propyl]amino]-2-[3-(trifluoromethyl)phenyl]acetate (6.50 g, 16.65 mmol) in DCM (15 mL). Resulting mixture was stirred at 20° C. for 1 hr. Then, the reaction mixture was poured into K₂CO₃ (aq), the DCM layer was separated and the aqueous layer was extracted with additional DCM, the combined organic layers was dried over Na₂SO₄, filtered and evaporated on vacuum. After solvent was distilled off, residue was heated to 80° C. under reduced pressure (approx. 15 torr) for 1 hr. Then, it was dissolved in boiling toluene (approx. 35-40 ml). Resulting solution was leaved for crystallization at 20° C. for 6 hr. Obtained crystals were filtered and dried, affording (6R)-6-methyl-3-[3-(trifluoromethyl)phenyl]piperazin-2-one (0.9 g, 3.49 mmol, 20.93% yield).

LCMS(ESI): [M]⁺ m/z: calcd 258.2; found 259.2; Rt=0.636 min.

### Step 4: Synthesis of (5R)-tert-butyl 5-methyl-3-oxo-2-(3-(trifluoromethyl)phenyl)piperazine-1-carboxylate

Prepared by Scheme 1.4, step 5. Yield: 1.3 g of crude.

LCMS(ESI): [M-t-Bu]⁺ m/z: calcd 302.2; found 303.2; Rt=1.211 min.

### Step 5: Synthesis of (5R)-tert-butyl 5-methyl-2-(3-(trifluoromethyl)phenyl)piperazine-1-carboxylate

Prepared by Scheme 1.4, step 6. Yield: 0.8 g (59.46%).

LCMS(ESI): [M-Boc]⁺ m/z: calcd 244.2; found 245.2; Rt=1.015 min.

### Step 6: Synthesis of (5R)-tert-butyl 5-methyl-4-(1-(trifluoromethyl)cyclopropanecarbonyl)-2-(3-(trifluoromethyl)phenyl)piperazine-1-carboxylate

Prepared by Scheme 1.4, step 7A. Yield: 0.56 g of crude.

LCMS(ESI): [M-t-Bu]⁺ m/z: calcd 424.2; found 425.2; Rt=1.441 min.

### Step 7: Synthesis of ((2R)-2-methyl-5-(3-(trifluoromethyl)phenyl)piperazin-1-yl)(1-(trifluoromethyl)cyclopropyl)methanone

tert-Butyl (2S,5R)-5-methyl-4-[1-(trifluoromethyl)cyclopropanecarbonyl]-2-[3-(trifluoromethyl) phenyl]piperazine-1-carboxylate (0.56 g, 1.17 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain [(2R,5S)-2-methyl-5-[3-(trifluoromethyl)phenyl]piperazin-1-yl]-[1-(trifluoromethyl)cyclopropyl]methanone (350 mg, 920.26 mol, 78.95% yield) which was used in the next step without further purification.

LCMS(ESI): [M]⁺ m/z: calcd 380.2; found 381.2; Rt=0.871 min.

### Step 8: Synthesis of N-(6-amino-5-methylpyridin-3-yl)-2-((2S,5R)-5-methyl-4-(1-(trifluoromethyl)cyclopropanecarbonyl)-2-(3-(trifluoromethyl)phenyl)piperazin-1-yl)-2-oxoacetamide

Prepared by Scheme 1.4, step 9A. Yield: 46.4 mg (18.09%).

HPLC conditions: Column: SunFire C18 100*19 mm, 5 microM; 2-10 min 0-80% MeCN/water; (loading pump 4 ml/min MeCN).

Compound 137: ¹H NMR (600 MHz, DMSO-d₆)₆ (ppm) 6 0.94-1.01 (m, 1H), 1.08-1.33 (m, 7H), 1.98-2.03 (m, 3H), 2.94-3.28 (m, 1H), 3.66-5.06 (m, 3H), 5.48-5.88 (m, 3H), 7.49-7.62 (m, 3H), 7.64-7.70 (m, 2H), 7.99-8.10 (m, 1H), 10.45-10.82 (m, 1H).

LCMS(ESI): [M]⁺ m/z: calcd 557.2; found 558.2; Rt=1.144 min.

### Example 103. Compound 78 N-(6-amino-5-methylpyridin-3-yl)-2-((2S,5R)-5-methyl-2-phenyl-4-(1-(trifluoromethyl)cyclopropanecarbonyl)piperazin-1-yl)-2-oxoacetamide

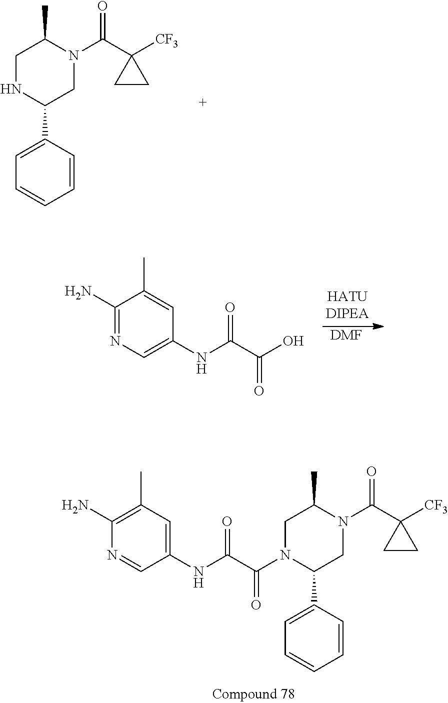

((2R,5S)-2-methyl-5-phenylpiperazin-1-yl)(1-(trifluoromethyl)cyclopropyl) methanone is prepared by general procedure scheme 1.4 step 8. Yield: 0.3 g (88.04%).

LCMS(ESI): [M]⁺ m/z: calcd 312.2; found 313.2; Rt=0.783 min.

Prepared by Scheme 1.4 step 9B. Yield: 35 mg (14.89%).

HPLC conditions: Column: YMC Triart C18 100*20 mm, 5 microM; 0-5 min 30-80% water/MeOH+0.1% NH₄OH 30 ml/min; (loading pump 4 ml/min MeOH).

Compound 78: ¹H NMR (500 MHz, DMSO-d₆) δ (ppm) 0.90-1.22 (m, 4H), 1.24-1.38 (m, 3H), 1.93-2.05 (m, 3H), 2.94-3.28 (m, 1H), 3.32-3.92 (m, 1H), 3.92-4.20 (m, 1H), 4.29-4.55 (m, 1H), 4.72-5.08 (m, 1H), 5.17-5.62 (m, 1H), 5.62-5.87 (m, 2H), 7.22-7.38 (m, 5H), 7.45-7.58 (m, 1H), 7.98-8.11 (m, 1H), 10.62 (br s, 1H).

LCMS(ESI): [M]⁺ m/z: calcd 489.2; found 490.2; Rt=2.111 min.

### Example 104. Compound 91 2-((2S,5R)-5-methyl-4-(1-methylcyclopropanecarbonyl)-2-phenylpiperazin-1-yl)-2-oxo-N-(1H-pyrazolo[4,3-c]pyridin-7-yl)acetamide

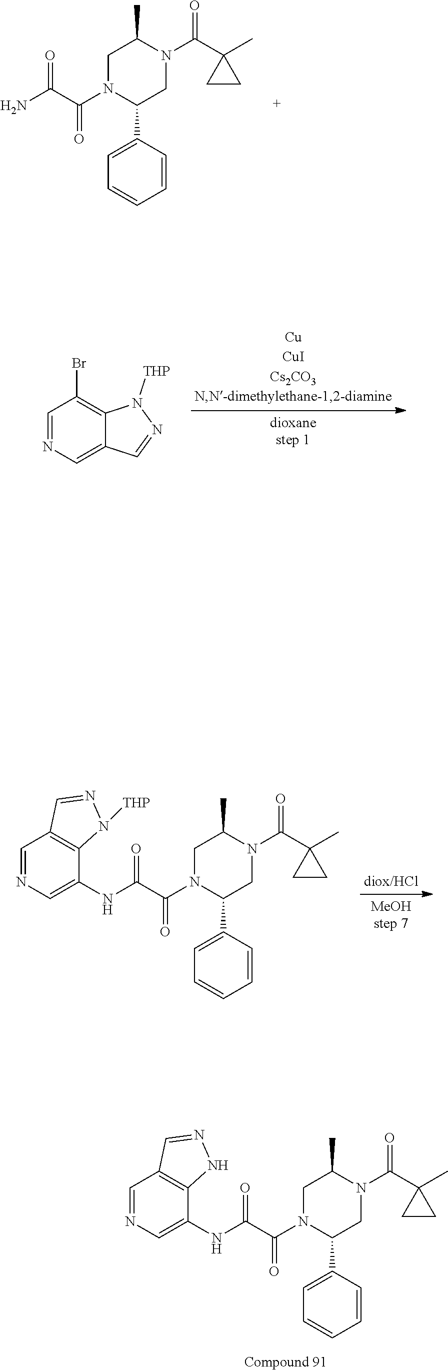

### Step 1: Synthesis of 2-((2S,5R)-5-methyl-4-(1-methylcyclopropanecarbonyl)-2-phenylpiperazin-1-yl)-2-oxo-N-(1-(tetrahydro-2H-pyran-2-yl)-1H-pyrazolo[4,3-c]pyridin-7-yl)acetamide

The synthesis of 2-((2S,5R)-5-methyl-4-(1-methylcyclopropanecarbonyl)-2-phenylpiperazin-1-yl)-2-oxoacetamide is prepared by Scheme 1.3 step 2. Yield: 0.37 mg (98.56%). LCMS(ESI): [M]⁺ m/z: calcd 329.2; found 330.2; Rt=1.025 min.

Prepared by sScheme 1.3 step 3A. Yield: 0.4 g of crude.

LCMS(ESI): [M]⁺ m/z: calcd 530.2; found 531.2; Rt=1.116 min.

### Step 2: Synthesis of 2-((2S,5R)-5-methyl-4-(1-methylcyclopropanecarbonyl)-2-phenylpiperazin-1-yl)-2-oxo-N-(1H-pyrazolo[4,3-c]pyridin-7-yl)acetamide (Compound 91)

The solution of 2-[(2S,5R)-5-methyl-4-(1-methylcyclopropanecarbonyl)-2-phenyl-piperazin-1-yl]-2-oxo-N-(1-tetrahydropyran-2-ylpyrazolo[4,3-c]pyridin-7-yl)acetamide (0.4 g, 753.84 umol) in MeOH (10 mL) and diox/HCl (10 mL) was stirred at 25° C. for 16 hr. Upon completion, the reaction mixture was concentrated under reduced pressure to obtain crude product. The obtained crude product was purified by reverse phase HPLC chromatography to afford product 2-[(2S,5R)-5-methyl-4-(1-methylcyclopropanecarbonyl)-2-phenyl-piperazin-1-yl]-2-oxo-N-(1H-pyrazolo[4,3-c]pyridin-7-yl)acetamide (33 mg, 73.91 umol, 9.80% yield). Compound 91: ¹H NMR (600 MHz, DMSO-d₆) δ (ppm) 0.47 (m, 2H), 0.72 (m, 2H), 1.06 (m, 4H), 1.27 (m, 3H), 4.06 (m, 2H), 4.65 (m, 2H), 5.73 (m, 1H), 7.34 (m, 6H), 8.79 (m, 3H), 11.45 (m, 1H), 13.52 (m, 1H).

LCMS(ESI): [M]⁺ m/z: calcd 446.2; found 447.2; Rt=2.107 min.

### Example 105. Compound 12 5-(2-((2S,5R)-2-(3-chloro-4-fluorophenyl)-4-isobutyryl-5-methylpiperazin-1-yl)-2-oxoacetamido)-2-methoxynicotinamide

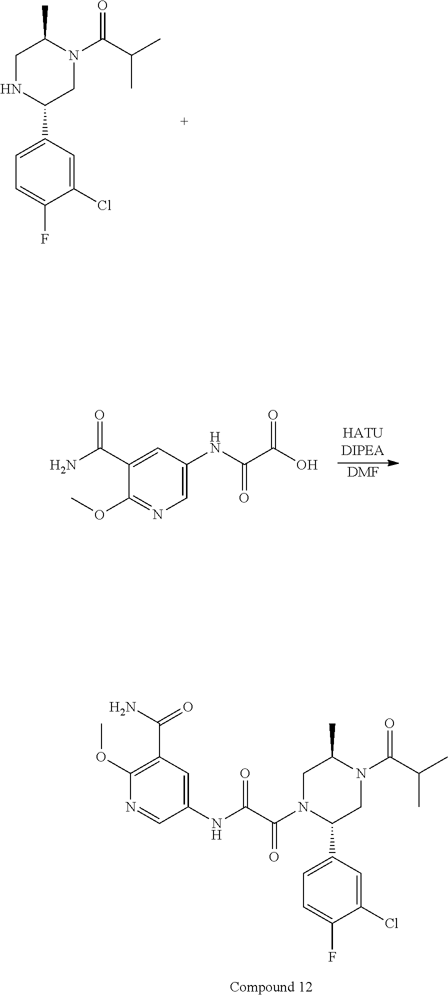

The synthesis of 1-((2R,5S)-5-(3-chloro-4-fluorophenyl)-2-methylpiperazin-1-yl)-2-methylpropan-1-one is given by the following procedure. tert-Butyl (2S,5R)-2-(3-chloro-4-fluoro-phenyl)-5-methyl-4-(2-methylpropanoyl)piperazine-1-carboxylate (0.27 g, 676.86 mol) was dissolved in DCM (10 mL) and TFA (385.89 mg, 3.38 mmol, 260.74 L) was added to reaction mixture at 0° C. then the solution was allowed to warm to 25° C. and stirred for 16 hr. Reaction mixture was diluted with DCM (30 ml) and washed by saturated aqueous NaHCO₃ (3*30 ml). The resulting organic phase was dried over Na₂SO₄ and evaporated under reduced pressure to afford 1-[(2R,5S)-5-(3-chloro-4-fluoro-phenyl)-2-methyl-piperazin-1-yl]-2-methyl-propan-1-one (0.12 g, 401.63 mol, 59.34% yield).

LCMS(ESI): [M]⁺ m/z: calcd 298.2; found 299.2; Rt=0.947 min.

Prepared by Scheme 1.4, step 9B. Yield: 36 mg (12.58%).

HPLC conditions: Column: Chromarex C18 100*19 mm, 5 microM; 0-5 min 20-45% MeCN/water+0.1% FA 30 ml/min; (loading pump 4 ml/min MeCN).

Compound 12: ¹H NMR (600 MHz, dmso) 6 0.69-1.30 (m, 9H), 2.63-3.28 (m, 3H), 3.66-3.84 (m, 1H), 3.92-3.97 (m, 3H), 3.98-4.93 (m, 2H), 5.33-5.69 (m, 1H), 7.22-7.58 (m, 3H), 7.68-7.78 (m, 2H), 8.34-8.56 (m, 2H), 10.84-11.31 (m, 1H).

LCMS(ESI): [M]⁺ m/z: calcd 519.2; found 520.2; Rt=3.005 min.

### Example 106. Compound 150 N-(6-amino-5-ethylpyridin-3-yl)-2-((2S,5R)-5-methyl-4-(1-(trifluoromethyl)cyclopropanecarbonyl)-2-(3-(trifluoromethyl)phenyl)piperazin-1-yl)-2-oxoacetamide

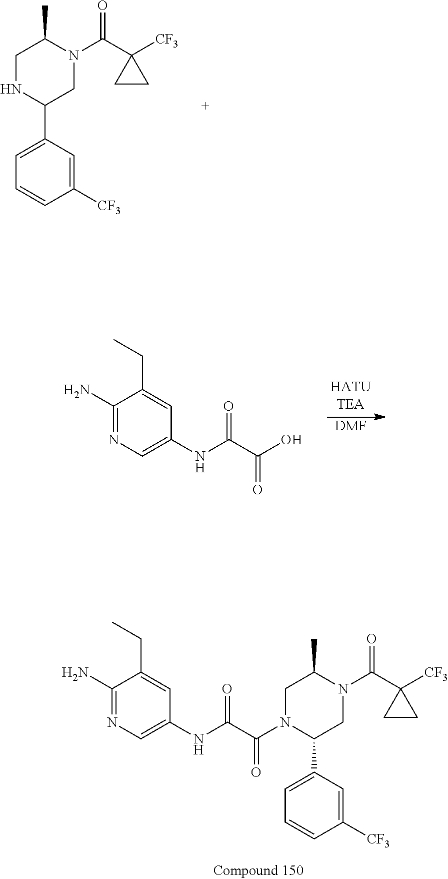

The synthesis of ((2R)-2-methyl-5-(3-(trifluoromethyl)phenyl)piperazin-1-yl)(1-(trifluoromethyl)cyclopropyl)methanone is given by the following procedure. tert-Butyl (2S,5R)-5-methyl-4-[1-(trifluoromethyl)cyclopropanecarbonyl]-2-[3-(trifluoromethyl) phenyl]piperazine-1-carboxylate (0.56 g, 1.17 mmol) was dissolved in TFA (3 mL) and DCM (3 mL) was added thereto. The resulting mixture was stirred for 1 hr. The reaction mixture was poured into aq·K₂CO₃ solution and the resulting mixture was extracted with DCM. Combined organic layers were dried over Na₂SO₄, filtered and evaporated to obtain [(2R,5S)-2-methyl-5-[3-(trifluoromethyl)phenyl]piperazin-1-yl]-[1-(trifluoromethyl)cyclopropyl]methanone (350 mg, 920.26 μmol, 78.95% yield) which was used in the next step without further purification.

LCMS(ESI): [M]⁺ m/z: calcd 380.2; found 381.2; Rt=0.871 min.

Prepared by Scheme 1.4 step 9A. Yield: 15.4 mg (5.86%).

HPLC conditions: Column: SunFire C18 100*19 mm, 5 microM; 2-10 min 0-80% MeCN-water; (loading pump 4 ml/min MeCN).

Compound 150:¹H NMR (600 MHz, DMSO-d₆)₆ (ppm) 6 0.96-1.00 (m, 1H), 1.08-1.13 (m, 4H), 1.13-1.35 (m, 8H), 2.93-3.05 (m, 1H), 3.36-5.06 (m, 3H), 5.46-5.92 (m, 3H), 7.50-7.63 (m, 3H), 7.64-7.69 (m, 2H), 8.02-8.11 (m, 1H), 10.58-10.78 (m, 1H).

LCMS(ESI): [M]⁺ m/z: calcd 571.2; found 572.2; Rt=1.178 min.

### Compounds of Formula III and III_5 Wherein R¹ is H (III′ and III_5′)

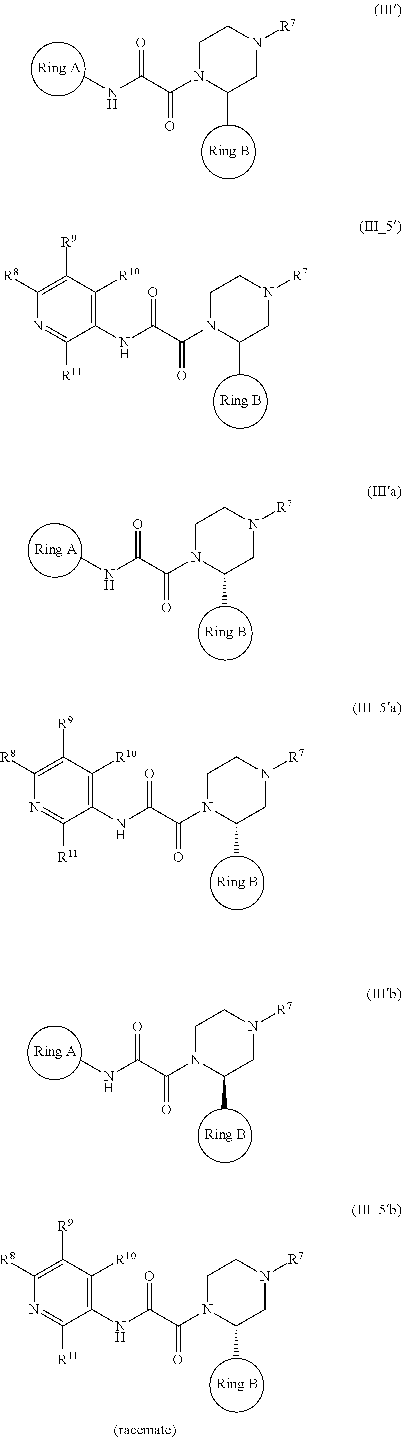

### Example 107. Compound 126 (S)—N-(4-amino-1H-pyrazolo[4,3-c]pyridin-7-yl)-2-(4-isobutyryl-2-phenylpiperazin-1-yl)-2-oxoacetamide

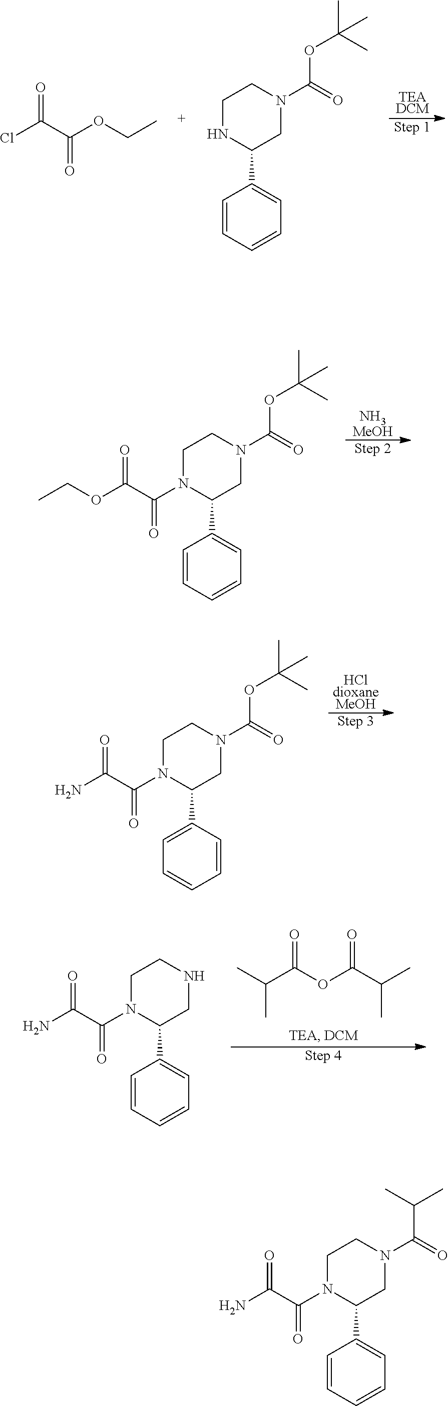

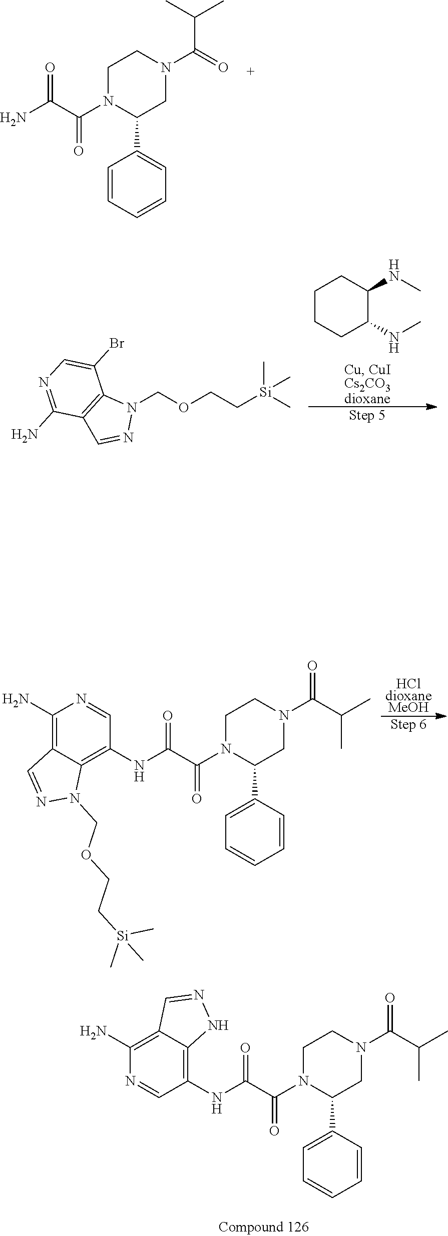

### Step 1: (S)-tert-butyl 4-(2-ethoxy-2-oxoacetyl)-3-phenylpiperazine-1-carboxylate

Ethyl 2-chloro-2-oxo-acetate (487.03 mg, 3.57 mmol, 398.55 L) was added dropwise to the solution of tert-butyl rac-(3S)-3-phenylpiperazine-1-carboxylate (935.83 mg, 3.57 mmol) and Triethylamine (541.44 mg, 5.35 mmol, 745.78 L) in Dichloromethane. After addition was complete, resulting mixture was stirred at 20° C. for 2 hr. Then, 10% aq. K₂CO₃ solution (15 ml) was added and stirring was continued for 10 min. After that, organic layer was separated, dried over K₂CO₃ and concentrated under reduced pressure, affording (S)-tert-butyl 4-(2-ethoxy-2-oxoacetyl)-3-phenylpiperazine-1-carboxylate (1.2 g, 3.31 mmol, 92.82% yield).

LCMS(ESI): [M-Boc+H]⁺ m/z: calcd 263.1; found 263.2; Rt=1.332 min.

### Step 2: (S)-tert-butyl 4-(2-amino-2-oxoacetyl)-3-phenylpiperazine-1-carboxylate

tert-butyl rac-(3R)-4-(2-ethoxy-2-oxo-acetyl)-3-phenyl-piperazine-1-carboxylate (1.2 g, 3.31 mmol) was dissolved in NH₃/methanol (30 mL) solution (10% by weight) and stirred overnight, then concentrated in vacuo to give (S)-tert-butyl 4-(2-amino-2-oxoacetyl)-3-phenylpiperazine-1-carboxylate (1.17 g, 3.51 mmol, 105.99% yield).

LCMS(ESI): [M−H]—m/z: calcd 332.2; found 332.0; Rt=1.212 min.

### Step 3: (S)-2-oxo-2-(2-phenylpiperazin-1-yl)acetamide

tert-butyl rac-(3S)-4-oxamoyl-3-phenyl-piperazine-1-carboxylate (1.17 g, 3.51 mmol) was dissolved in mixture of Methanol (5 mL) and HCl in Dioxane (70.19 mmol), then stirred for 1 hr. The reaction mixture was concentrated in vacuo, the residue was treated with aq. solution of NaHCO₃ and desired product was extracted with DCM (2×40 mL), dried over Na₂SO₄, evaporated in vacuo to give (S)-2-oxo-2-(2-phenylpiperazin-1-yl)acetamide (0.92 g, 3.41 mmol, 97.19% yield, HCl).

LCMS(ESI): [M+H]⁺ m/z: calcd 234.1; found 234.2; Rt=0.316 min.

### Step 4: (S)-2-(4-isobutyryl-2-phenylpiperazin-1-yl)-2-oxoacetamide

2-methylpropanoyl 2-methylpropanoate (263.92 mg, 1.67 mmol, 276.65 L) was added dropwise to the solution of 2-oxo-2-[rac-(2S)-2-phenylpiperazin-1-yl]acetamide (0.45 g, 1.67 mmol, HCl) and Triethylamine (506.46 mg, 5.01 mmol, 697.61 L) in Dichloromethane. After addition was complete, resulting mixture was stirred at 20° C. for 2 hr. Then, 10% aq. K₂CO₃ solution (15 ml) was added and stirring was continued for 10 min. After that, organic layer was separated, dried over K₂CO₃ and concentrated under reduced pressure, affording (S)-2-(4-isobutyryl-2-phenylpiperazin-1-yl)-2-oxoacetamide (0.35 g, 1.15 mmol, 69.16% yield).

LCMS(ESI): [M+H]⁺ m/z: calcd 304.2; found 304.2; Rt=0.891 min.

### Step 5: (S)—N-(4-amino-1-((2-(trimethylsilyl)ethoxy)methyl)-1H-pyrazolo[4,3-c]pyridin-7-yl)-2-(4-isobutyryl-2-phenylpiperazin-1-yl)-2-oxoacetamide

7-bromo-1-(2-trimethylsilylethoxymethyl)pyrazolo[4,3-c]pyridin-4-amine (0.2 g, 582.59 mol), 2-oxo-2-[rac-(2S)-4-(2-methylpropanoyl)-2-phenyl-piperazin-1-yl]acetamide (176.73 mg, 582.59 mol), Copper (I) iodide (110.95 mg, 582.59 mol, 19.74 μL), Cesium carbonate (379.64 mg, 1.17 mmol) and (1R,2R)—N₁,N₂-dimethylcyclohexane-1,2-diamine (124.30 mg, 873.89 mol) were mixed in Dioxane under argon, and then stirred overnight at 100° C. for 12 hr in vial. Reaction mixture was filtered, then evaporated. Crude product was dissolved in 2 ml DMSO and subjected to HPLC to give (S)—N-(4-amino-1-((2-(trimethylsilyl)ethoxy)methyl)-1H-pyrazolo[4,3-c]pyridin-7-yl)-2-(4-isobutyryl-2-phenylpiperazin-1-yl)-2-oxoacetamide (0.027 g, 47.73 mol, 8.19% yield).

LCMS(ESI): [M+H]⁺ m/z: calcd 566.3; found 566.2; Rt=1.258 min.

### Step 6: The synthesis of (S)—N-(4-amino-1H-pyrazolo[4,3-c]pyridin-7-yl)-2-(4-isobutyryl-2-phenylpiperazin-1-yl)-2-oxoacetamide

N-[4-amino-1-(2-trimethylsilylethoxymethyl)pyrazolo[4,3-c]pyridin-7-yl]-2-oxo-2-[rac-(2S)-4-(2-methylpropanoyl)-2-phenyl-piperazin-1-yl]acetamide (0.027 g, 47.73 mol) was dissolved in MeOH (1 mL) and diox/HCl (954.51 mol, 1 mL) was added thereto. Then it was stirred at rt for 2 hr. The reaction mixture was evaporated to afford (S)—N-(4-amino-1H-pyrazolo[4,3-c]pyridin-7-yl)-2-(4-isobutyryl-2-phenylpiperazin-1-yl)-2-oxoacetamide (0.0076 g, 17.45 mol, 36.57% yield).

LCMS(ESI): [M+H]⁺ m/z: calcd 436.2; found 436.2; Rt=0.762 min.

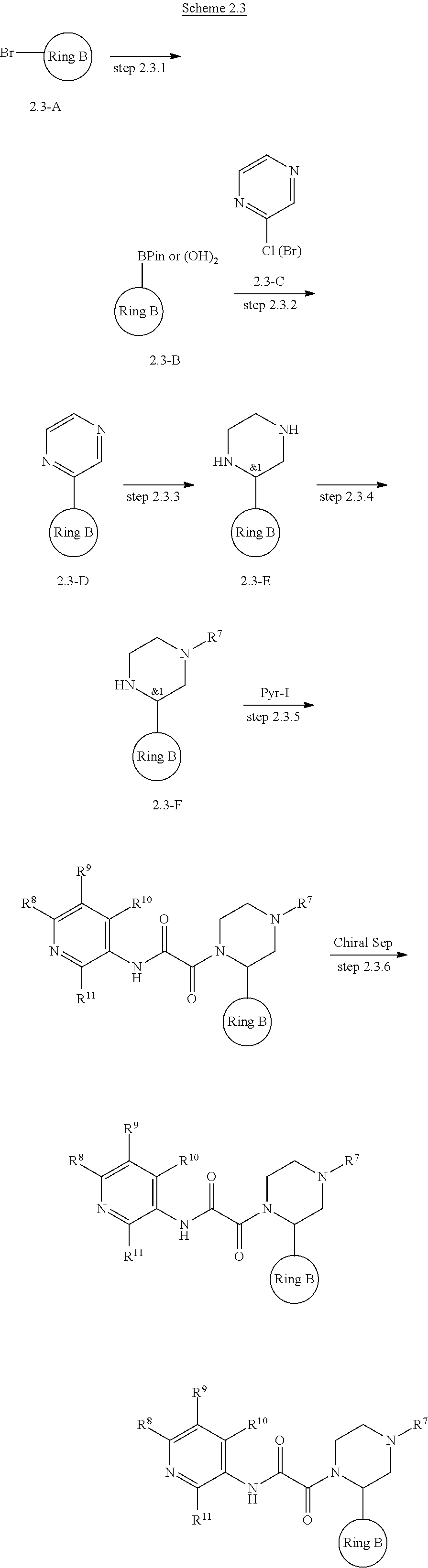

General Procedures for Scheme 2.3

### Step 2.3.1: Synthesis of 2.3-B

Compound 2.3-B was prepared from 2.3-A according to common literature procedures.

### Step 2.3.2: Synthesis of 2.3-D

A mixture of 2.3-B (1 eq), pyrazine 2.3—C(1.5 eq) and Sodium carbonate (2 eq) in Dioxane and Water was evacuated and then backfilled with argon. This operation was repeated three times, then Pd(dppf)Cl₂DCM (0.05 eq) was added in an inert atmosphere. After stirring at 90° C. for 48 hr, the reaction mixture was cooled down and filtered. The filter cake was washed with 1,4-dioxane and discarded. The filtrate was concentrated under reduced pressure to afford crude product. The residue was purified by column chromatography (SiO₂; chloroform/acetonitrile gradient (0-100% acetonitrile)) to afford 2.3-D.

### Step 2.3.3: Synthesis of 2.3-E

To a solution of 2.3-D (1 eq) in a mixture of methanol and acetic acid Palladium, 10% on carbon (1 eq) was added. The reaction mixture was hydrogenated at 50° C. for 48 hr, then cooled down and filtered. Hydrogen chloride solution 4.0M in dioxane (1 eq) was added to the filtrate and the resulting solution was concentrated in vacuo. The residue was diluted with MTBE, stirred for 0.5 hr and filtered. The filter cake was washed with MTBE and dried in vacuo to afford crude 2.3-E.

### Step 2.3.4A: Synthesis of 2.3-F

To a stirring solution of 2.3-E (1 eq) in THF, anhydride (1.175 eq), TEA (1.5 eq) was added at 0° C. After stirring for 8 hr at 0° C., the reaction mixture was concentrated under reduced pressure to give 2.3-F.

### Step 2.3.4B: Synthesis of 2.3-F

RCOCI (1,4 eq) was added slowly to a stirred mixture of 2,3-E (1 eq) and Triethylamine (6 eq) in DCM at −40° C. The reaction mixture was stirred at −40° C. for 1 hr, and then allowed to warm to 25° C. and stirred at the temperature for 12 hr. The obtained mixture was concentrated in vacuo and the residue was purified by gradient chromatography (MTBE-MeOH) to afford 2.3-F.

### Step 2.3.5: Synthesis of Racemic Final Product

To a solution of 2.3-F (1 eq), 2.3-G (1 eq) and Triethylamine (5 eq) in DMF HATU (1.2 eq) was added portionwise. After stirring at 25° C. for 4 hr, the reaction mixture was diluted with water and extracted with EtOAc. The combined organic layer was washed with brine, dried over Na₂SO₄ and concentrated in vacuo to give racemic product.

### Step 2.3.6: Synthesis of Enantiopure Products

Racemic product was subjected to chiral HPLC to give enantiopure products.

### Example 108. Compound 123 N-(6-amino-5-ethyl-3-pyridyl)-2-[(2R)-2-(3-chloro-4-fluoro-phenyl)-4-[1-(trifluoromethyl)cyclopropanecarbonyl]piperazin-1-yl]-2-oxo-acetamide and Compound 47 N-(6-amino-5-ethyl-3-pyridyl)-2-[(2S)-2-(3-chloro-4-fluoro-phenyl)-4-[1-(trifluoromethyl)cyclopropanecarbonyl]piperazin-1-yl]-2-oxo-acetamide

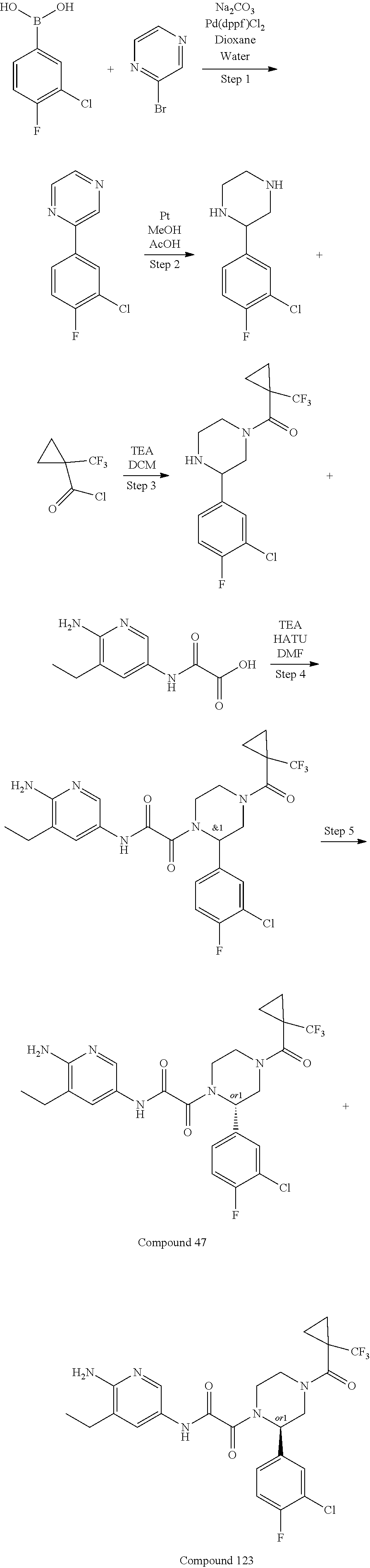

### Step 1: The synthesis of 2-(3-chloro-4-fluoro-phenyl)pyrazine

Prepared according to Scheme 2.3, step 2.3.2. Yield: 29 g, 139.01 mmol, 96.95%.

LCMS(ESI): [M+H]⁺ m/z: calcd 209.03; found 209.0; Rt=1.226.

### Step 2. The synthesis of rac-(2R)-2-(3-chloro-4-fluoro-phenyl)piperazine

Prepared according to Scheme 2.3, step 2.3.3 (Pt was used instead of Pd/C). Yield: 6 g, 6.54 mmol, 91.73%. LCMS(ESI): [M+H]⁺ m/z: calcd 215.09; found 215.2; Rt=0.373.

### Step 3: The synthesis of rac-[(3R)-3-(3-chloro-4-fluoro-phenyl)piperazin-1-yl]-[1-(trifluoromethyl)cyclopropyl]methanone

Prepared according to Scheme 2.3, step 2.3.4B; crude (70% by LCMS).

LCMS(ESI): [M+H]⁺ m/z: calcd 351.11; found 351.0; Rt=0.979.

### Step 4: The synthesis of rac-N-(6-amino-5-ethyl-3-pyridyl)-2-[(2R)-2-(3-chloro-4-fluoro-phenyl)-4-[1-(trifluoromethyl)cyclopropanecarbonyl]piperazin-1-yl]-2-oxo-acetamide

Prepared according to Scheme 2.3, step 2.3.5; 145 mg, 267.56 umol, 26.81% yield.

HPLC conditions: SYSTEM 50-50-70% 0-1-6 min H₂O/MeOH/0.1% NH₄OH, flow: 30 ml/min (loading pump 4 ml/min methanol) target mass 541 column: YMC Triart C18 100×20 mm, 5 um).

LCMS(ESI): [M+H]⁺ m/z: calcd 542.18; found 542.0; Rt=2.949.

### Step 5: The synthesis of rel-N-(6-amino-5-ethyl-3-pyridyl)-2-[(2R)-2-(3-chloro-4-fluoro-phenyl)-4-[1-(trifluoromethyl)cyclopropanecarbonyl]piperazin-1-yl]-2-oxo-acetamide and rel-N-(6-amino-5-ethyl-3-pyridyl)-2-[(2S)-2-(3-chloro-4-fluoro-phenyl)-4-[1-(trifluoromethyl)cyclopropanecarbonyl]piperazin-1-yl]-²-oxo-acetamide

Prepared according to Scheme 2.3, step 2.3.6.

HPLC conditions Column: Chiralpak AD-H-Ill (250*20, 5mkm), Hexane-IPA-MeOH, 60-20-20, Flow Rate: 12 mL/min; Column Temperature: 24° C.; Wavelength: 205 nm, 270 nm, 312 nm), RetTime (isomer A)=39.72 min; RetTime (isomer B)=53.86 min.

N-(6-amino-5-ethyl-3-pyridyl)-2-[(2S)-2-(3-chloro-4-fluoro-phenyl)-4-[1-(trifluoromethyl)cyclopropanecarbonyl]piperazin-1-yl]-2-oxo-acetamide (Compound 47) Yield: 41 mg, 75.66 umol, 82.83% yield; RetTime=39.72 min.

1H NMR (600 MHz, DMSO) δ 1.04-1.35 (m, 8H), 2.38-2.43 (m, 2H), 2.80-3.21 (m, 2H), 3.45-5.12 (m, 3H), 5.37-5.78 (m, 3H), 7.23-7.37 (m, 1H), 7.37-7.52 (m, 3H), 7.97-8.09 (m, 1H), 10.51-10.71 (m, 1H).

LCMS(ESI): [M+H]⁺ m/z: calcd 542.18; found 542.2; Rt=3.045.

N-(6-amino-5-ethyl-3-pyridyl)-2-[(2R)-2-(3-chloro-4-fluoro-phenyl)-4-[I-(trifluoromethyl)cyclopropanecarbonyl]piperazin-1-yl]-2-oxo-acetamide (Compound 123) Yield: 48 mg, 88.57 umol, 96.97% yield; RetTime=53.86 min.

1H NMR (600 MHz, DMSO) δ 1.05-1.32 (m, 8H), 2.37-2.42 (m, 2H), 2.82-3.20 (m, 2H), 3.80-5.05 (m, 3H), 5.36-5.77 (m, 3H), 7.23-7.36 (m, 1H), 7.39-7.52 (m, 3H), 7.97-8.10 (m, 1H), 10.49-10.69 (m, 1H).

LCMS(ESI): [M+H]⁺ m/z: calcd 542.18; found 542.2; Rt=3.029.

Compounds of Formula Iii Wherein R¹ IS H, AND R² is Me (III″)

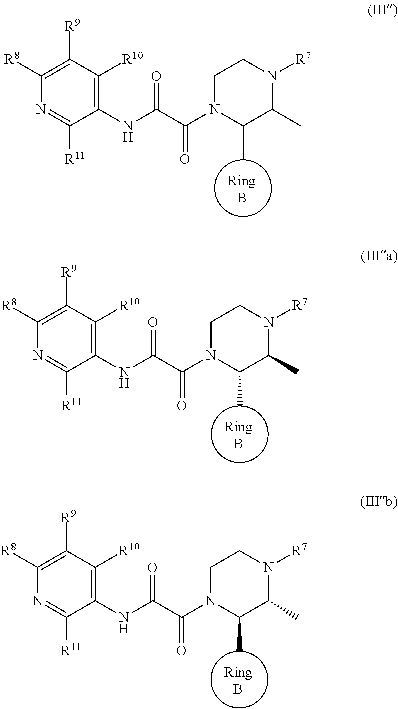

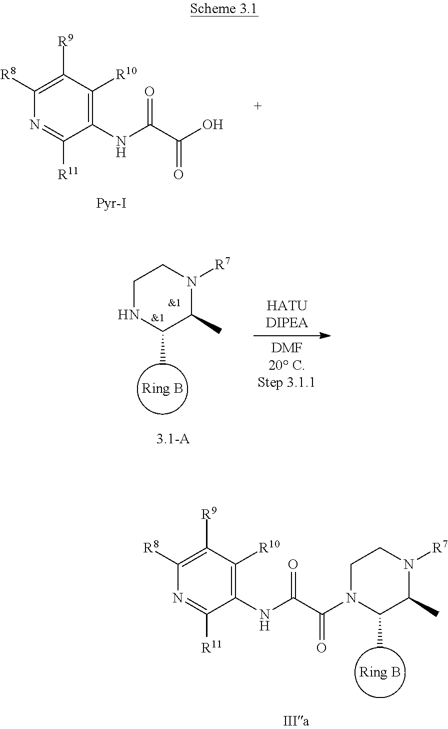

### Example 109. Compound 118 N-(6-amino-5-methyl-3-pyridyl)-2-[2-(4-fluorophenyl)-3-methyl-4-[1-(trifluoromethyl)cyclopropanecarbonyl]piperazin-1-yl]-2-oxo-acetamide

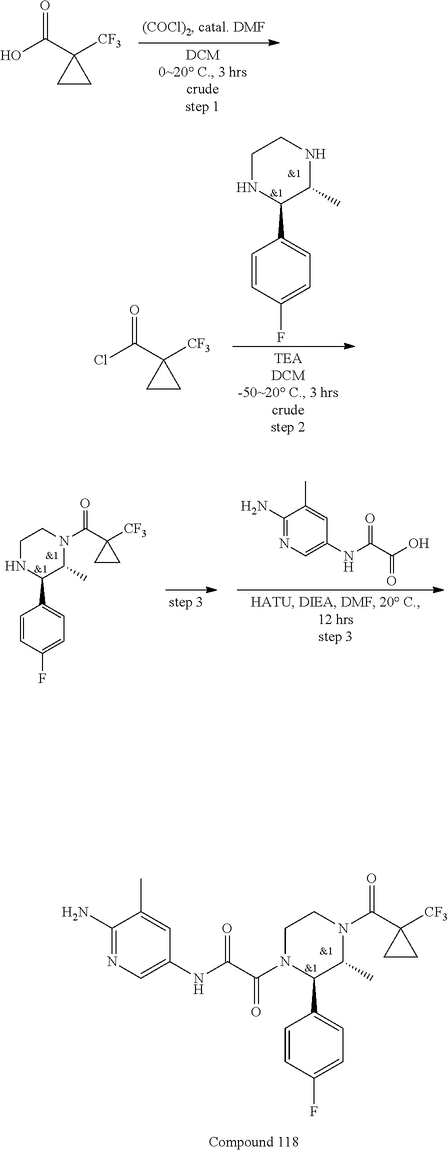 [SMILES: O=C(O)C1(C(F)(F)F)CC1 ; O=C(Cl)C1(C(F)(F)F)CC1 ; C[C@H]1NCCN[C@@H]1c1ccc(F)cc1 ; C[C@@H]1[C@@H](c2ccc(F)cc2)NCCN1C(=O)C1(C(F)(F)F)CC1 ; Cc1cc(NC(=O)C(=O)O)cnc1N ; Cc1cc(NC(=O)C(=O)N2CCN(C(=O)C3(C(F)(F)F)CC3)[C@H](C)[C@H]2c2ccc(F)cc2)cnc1N]

### Step 1: The Synthesis of 1-(trifluoromethyl)cyclopropanecarbonyl chloride

To a mixture of 1-(trifluoromethyl)cyclopropanecarboxylic acid (170 mg, 1.1 mmol) and DMF (10 mg, 0.14 mmol) in DCM (3 mL) was added oxalyl dichloride (160 mg, 1.26 mmol) and the mixture was stirred at 0° C. for 1 hour, then warmed to 20° C. and stirred for 2 hours. The reaction mixture was concentrated under reduce pressure to give 1-(trifluoromethyl)cyclopropanecarbonyl chloride (211 mg, crude) as a brown oil which was used directly to the next step without further purification.

### Step 2: The Synthesis of [3-(4-fluorophenyl)-2-methyl-piperazin-1-yl]-[-(trifluoromethyl)cyclopropyl]methanone

To a mixture of 2-(4-fluorophenyl)-3-methyl-piperazine (190 mg, 0.98 mmol) and TEA (313 mg, 3.09 mmol) in DCM (10 mL) was added 1-(trifluoromethyl)cyclopropanecarbonyl chloride (170 mg, 0.985 mmol) at −50° C. and the mixture was stirred at −50° C. for 1 hour, then warmed to 20° C. and stirred for 2 hours. The mixture was concentrated under reduce pressure to give [3-(4-fluorophenyl)-2-methyl-piperazin-1-yl]-[1-(trifluoromethyl)cyclopropyl]methanone (290 mg, crude) as a brown oil which was used directly to the next step without further purification. LCMS (ESI) [M+H]Y m/z: calcd 331.1, found 331.1.

### Step 3: The Synthesis of rac-N-(6-amino-5-methyl-3-pyridyl)-2-[2-(4-fluorophenyl)-3-methyl-4-[1-(trifluoromethyl)cyclopropanecarbonyl]piperazin-1-yl]-2-oxo-acetamide

To a mixture of 2-[(6-amino-5-methyl-3-pyridyl)amino]-2-oxo-acetic acid (230 mg, 1.18 mmol) and [3-(4-fluorophenyl)-2-methyl-piperazin-1-yl]-[1-(trifluoromethyl)cyclopropyl]methanone (300 mg, 0.91 mmol) in DMF (2 mL) and DCM (2 mL) were added HATU (480 mg, 1.26 mmol) and DIEA (580 mg, 5.74 mmol) and the mixture was stirred at 20° C. for 12 hours. The mixture was concentrated under reduce pressure and the residue was preparative HPLC purification (Instrument: Gilson GX-281 Liquid Handler, Gilson 322 Pump, Gilson 156 UV Detector; Column: Durashell 150×25 mm×5 m; Mobile phase A: water(0.05% NH₃H₂O+10 mM NH₄HCO₃)-ACN; Mobile phase B: MeCN; Gradient: B from 23% to 53% in 9.5 min, hold 100% B for 2.0 min; Flow Rate: 25 mL/min; Column Temperature: 30° C.; Wavelength: 220 nm, 254 nm) to afford N-(6-amino-5-methyl-3-pyridyl)-2-[2-(4-fluorophenyl)-3-methyl-4-[1-(trifluoromethyl)cyclopropanecarbonyl]piperazin-1-yl]-2-oxo-acetamide (51 mg, 11.1% yield, racemic mixture with trans relative chemistry) as a white solid. ¹H NMR (400 MHz, DMSO-d₆) δ ppm 10.58-10.79 (m, 1H), 8.08 (d, J 2.3 Hz, 1H), 7.55 (br d, J 1.8 Hz, 1H), 7.34-7.48 (m, 1H), 7.22 (br d, J 8.8 Hz, 3H), 5.67 (d, J 3.5 Hz, 2H), 5.20-5.36 (m, 1H), 5.10-5.14 (m, 1H), 3.77-4.28 (m, 2H), 3.38-3.51 (m, 1H), 2.61-3.00 (m, 1H), 2.05 (d, J 9.0 Hz, 3H), 1.18-1.35 (m, 6H), 0.98-1.10 (m, 1H); ¹⁹F NMR (377 MHz, DMSO-d₆)₆ ppm -115.07, -66.432; LCMS (ESI) [M+H]⁺ m/z: calcd 508.1, found 508.1; HPLC: 96.76%®220 nm, 100%®254 nm; racemic mixture with trans relative chemistry.

### Example 11.0. (Compound 163)N-(4-Amino-2H-pyrazolo[4,3-c]pyridin-7-yl)-2-oxo-2-[rac-(2S,5R)-4-cyclobutyl-5-methyl-2-phenyl-piperazin-1-yl]acetamide

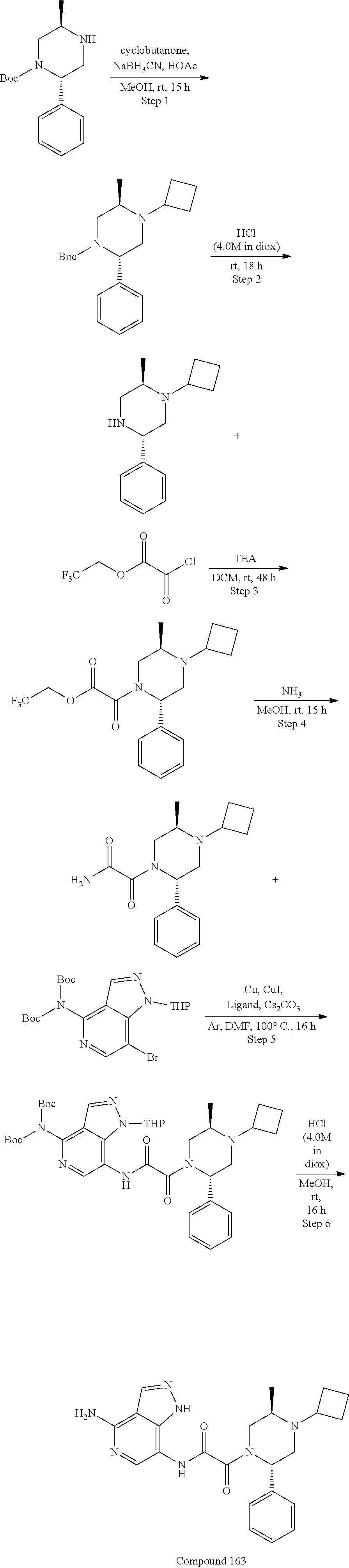

### Step 1: The Synthesis of tert-Butyl rac-(2S,5R)-4-cyclobutyl-5-methyl-2-phenyl-piperazine-1-carboxylate

Cyclobutanone (443.81 mg, 6.33 mmol, 473.15 L) and Acetic acid (217.29 mg, 3.62 mmol, 207.14 L) were added to a stirred solution of tert-butyl rac-(2S,5R)-5-methyl-2-phenyl-piperazine-1-carboxylate (0.5 g, 1.81 mmol) in MeOH (9.17 mL) at 25° C. The resulting mixture was stirred at 25° C. for 0.5 hr, then Sodium cyanoborohydride (227.38 mg, 3.62 mmol, 211.71 L) was added in one portion at 25° C. (foaming!). The reaction mixture was stirred at 25° C. for 14 hr, and then concentrated in vacuo. The residue was diluted with 10% aqueous sodium hydroxide solution (40 mL) and extracted with dichloromethane (3*20 mL). The combined organic extracts were dried over sodium sulphate and concentrated in vacuo to afford tert-butyl rac-(2S,5R)-4-cyclobutyl-5-methyl-2-phenyl-piperazine-1-carboxylate (0.5 g, crude).

LCMS(ESI): [M+H]⁺ m/z: calcd 331.2; found 331.2; Rt=0.875 min.

### Step 2: The Synthesis of rac-(2R,5S)-1-Cyclobutyl-2-methyl-5-phenyl-piperazine

tert-Butyl rac-(2S,5R)-4-cyclobutyl-5-methyl-2-phenyl-piperazine-1-carboxylate (0.5 g, 1.51 mmol) was dissolved in Diox/HCl (20 mL) and the resulting mixture was stirred at 25° C. for 16 hr. Upon completion of the reaction, solvent was evaporated, 20 mL water was added and the resulting mixture was basified with K₂CO₃ to alkaline pH, then mixture was extracted with DCM (3*10 mL), combined organic layers were dried over Na₂SO₄, filtered and concentrated in vacuo to afford rac-(2R,5S)-1-cyclobutyl-2-methyl-5-phenyl-piperazine (300 mg, 1.30 mmol, 86.08% yield).

LCMS(ESI): [M+H]⁺ m/z: calcd 231.2; found 231.2; Rt=0.501 min.

### Step 3: The Synthesis of 2,2,2-Trifluoroethyl 2-oxo-2-[rac-(2S,5R)-4-cyclobutyl-5-methyl-2-phenyl-piperazin-1-yl]acetate

rac-(2R,5S)-1-Cyclobutyl-2-methyl-5-phenyl-piperazine (0.3 g, 1.30 mmol) and TEA (197.68 mg, 1.95 mmol, 272.29 L) were mixed together in DCM (19.91 mL) and the resulting solution was cooled to 5° C. in an ice bath. 2,2,2-Trifluoroethyl 2-chloro-2-oxo-acetate (322.54 mg, 1.69 mmol) was added dropwise to the previous solution and the resulting mixture was allowed to warm to rt and stirred for 16 hr. Upon completion of the reaction, the mixture was washed with brine (2*20 mL), organic layer was dried over Na₂SO₄, filtered and concentrated in vacuo to affording 2,2,2-trifluoroethyl 2-oxo-2-[rac-(2S,5R)-4-cyclobutyl-5-methyl-2-phenyl-piperazin-1-yl]acetate (0.5 g, crude).

LCMS(ESI): [M+H]⁺ m/z: calcd 385.2; found 385.2; Rt=0.872 min.

### Step 4: The Synthesis of 2-Oxo-2-[rac-(2S,5R)-4-cyclobutyl-5-methyl-2-phenyl-piperazin-1-yl]acetamide

2,2,2-Trifluoroethyl 2-oxo-2-[rac-(2S,5R)-4-cyclobutyl-5-methyl-2-phenyl-piperazin-1-yl]acetate (0.5 g, 1.30 mmol) was dissolved in MeOH/NH₃ (20 mL) and the resulting mixture was stirred at 25° C. for 15 hr. Upon completion of the reaction, solvent was concentrated in vacuo to afford 2-oxo-2-[rac-(2S,5R)-4-cyclobutyl-5-methyl-2-phenyl-piperazin-1-yl]acetamide (380 mg, crude).

LCMS(ESI): [M+H]⁺ m/z: calcd 302.2; found 302.2; Rt=0.694 min.

### Step 5: The Synthesis of tert-Butyl N-tert-butoxycarbonyl-N-[7-[[2-oxo-2-[rac-(2S,5R)-4-cyclobutyl-5-methyl-2-phenyl-piperazin-1-yl]acetyl]amino]-2-tetrahydropyran-2-yl-pyrazolo[4,3-c]pyridin-4-yl]carbamate

A mixture of 2-oxo-2-[rac-(2S,5R)-4-cyclobutyl-5-methyl-2-phenyl-piperazin-1-yl]acetamide (180 mg, 597.25 mol), tert-butyl N-(7-bromo-2-tetrahydropyran-2-yl-pyrazolo[4,3-c]pyridin-4-yl)-N-tert-butoxycarbonyl-carbamate (297.06 mg, 597.25 mol), Cu (7.59 mg, 119.45 μmol), CuI (113.75 mg, 597.25 mol, 20.24 μL), Cesium carbonate (389.19 mg, 1.19 mmol) and (1S,2S)—N,N′-Bis-methyl-1,2-cyclohexane-diamine (127.43 mg, 895.87 μmol, 141.28 L) in DMF (4.02 mL) was stirred in a sealed vial under argon at 100° C. for 16 hr. Upon completion of the reaction, mixture was filtered, precipitate was washed with CHCl₃, mother liquid was separated and concentrated in vacuo to dryness. Crude product tert-butyl N-tert-butoxycarbonyl-N-[7-[[2-oxo-2-[rac-(2S,5R)-4-cyclobutyl-5-methyl-2-phenyl-piperazin-1-yl]acetyl]amino]-2-tetrahydropyran-2-yl-pyrazolo [4,3-c]pyridin-4-yl]carbamate (0.8 g, crude) was used in the next step without any purification.

LCMS(ESI): [M+H]⁺ m/z: calcd 718.2; found 718.2; Rt=1.372 min.

### Step 6: The Synthesis of N-(4-Amino-2H-pyrazolo[4,3-c]pyridin-7-yl)-2-oxo-2-[rac-(2S,5R)-4-cyclobutyl-5-methyl-2-phenyl-piperazin-1-yl]acetamide (Compound 163)

tert-Butyl N-tert-butoxycarbonyl-N-[7-[[2-oxo-2-[rac-(2S,5R)-4-cyclobutyl-5-methyl-2-phenyl-piperazin-1-yl]acetyl]amino]-2-tetrahydropyran-2-yl-pyrazolo[4,3-c]pyridin-4-yl]carbamate (0.8 g, 1.11 mmol) was dissolved in a mixture of MeOH (3 mL) and Diox/HCl (3 mL) and the resulting mixture was stirred at 25° C. for 16 hr. After that time, precipitate was filtered off, mother liquid was evaporated in vacuo to obtain crude product. Crude product was subjected by HPLC (column: Chromatorex 18 SMB100-5T 100×19 mm, 5 um; mobile phase: 5-5-25% 0-1-5 min H₂O/MeCN/0.1% FA, flow rate: 30 mL/min (loading pump 4 mL/min H₂O)) to obtain N-(4-amino-2H-pyrazolo[4,3-c]pyridin-7-yl)-2-oxo-2-[rac-(2S,5R)-4-cyclobutyl-5-methyl-2-phenyl-piperazin-1-yl]acetamide (61.2 mg, 127.62 μmol, 11.45% yield, HCOOH)

1H NMR (600 MHz, dmso) 6-0.07-1.00 (m, 3H), 1.41-1.86 (m, 5H), 1.88-1.96 (m, 1H), 1.98-2.08 (m, 1H), 2.70-3.01 (m, 3H), 3.07-3.13 (m, 1H), 3.72-4.15 (m, 1H), 4.87-5.62 (m, 1H), 6.44-7.28 (m, 3H), 7.28-7.54 (m, 4H), 7.55-7.77 (m, 1H), 8.12 (s, 1H), 8.19-8.40 (m, 1H), 9.60-10.69 (m, 1H), 11.69-13.96 (m, 2H).

LCMS(ESI): [M+H]⁺ m/z: calcd 434.2; found 434.2; Rt=1.481 min.

### Example 1.11. (Compound 161)N-(4-Amino-1H-pyrazolo[4,3-c]pyridin-7-yl)-2-oxo-2-[rac-(2S,5R)-5-methyl-4-(1-methylcyclopropanecarbonyl)-2-phenyl-piperazin-1-yl]acetamide

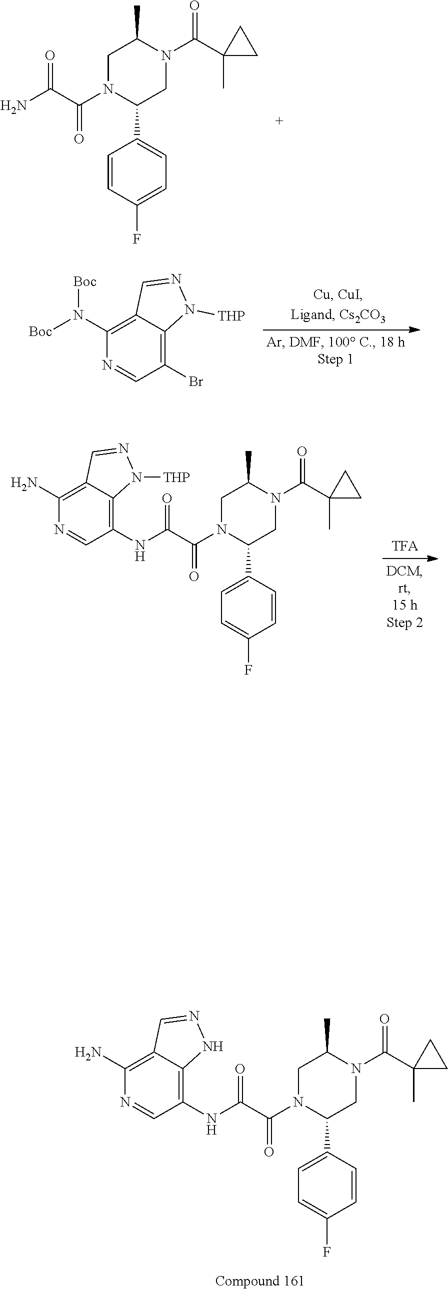

### Step 1: The Synthesis of tert-Butyl N-tert-butoxycarbonyl-N-[7-[[2-oxo-2-[rac-(2S,5R)-5-methyl-4-(1-methylcyclopropanecarbonyl)-2-phenyl-piperazin-1-yl]acetyl]amino]-2-tetrahydropyran-2-yl-pyrazolo[4,3-c]pyridin-4-yl]carbamate

A mixture of 2-oxo-2-[rac-(2S,5R)-5-methyl-4-(1-methylcyclopropanecarbonyl)-2-phenyl-piperazin-1-yl]acetamide (0.16 g, 485.74 mol), tert-butyl N-(7-amino-2-tetrahydropyran-2-yl-pyrazolo[4,3-c]pyridin-4-yl)-N-tert-butoxycarbonyl-carbamate (210.57 mg, 485.74 mol), Cu (6.17 mg, 97.15 mol), CuI (92.51 mg, 485.74 mol, 16.46 μL), Cesium carbonate (316.53 mg, 971.49 mol) and (1S,2S)—N,N′-Bis-methyl-1,2-cyclohexane-diamine (103.64 mg, 728.61 μmol, 114.90 L) in DMF (4 mL) was stirred in a sealed vial under argon at 100° C. for 18 hr. Upon completion of the reaction, mixture was filtered, precipitate was washed with CHCl₃, mother liquid was separated and concentrated in vacuo to dryness. Crude product tert-butyl N-tert-butoxycarbonyl-N-[7-[[2-oxo-2-[rac-(2S,5R)-5-methyl-4-(1-methylcyclopropanecarbonyl)-2-phenyl-piperazin-1-yl]acetyl]amino]-2-tetrahydropyran-2-yl-pyrazolo[4,3-c]pyridin-4-yl]carbamate (0.6 g, crude) was used in the next step without any purification.

LCMS(ESI): [M+H]⁺ m/z: calcd 746.2; found 746.2; Rt=1.573 min.

### Step 2: The Synthesis of N-(4-Amino-1H-pyrazolo[4,3-c]pyridin-7-yl)-2-oxo-2-[rac-(2S,5R)-5-methyl-4-(1-methylcyclopropanecarbonyl)-2-phenyl-piperazin-1-yl]acetamide (Compound 161)

tert-Butyl N-tert-butoxycarbonyl-N-[7-[[2-oxo-2-[rac-(2S,5R)-5-methyl-4-(1-methylcyclopropanecarbonyl)-2-phenyl-piperazin-1-yl]acetyl]amino]-2-tetrahydropyran-2-yl-pyrazolo[4,3-c]pyridin-4-yl]carbamate (0.6 g, 804.44 mol) was dissolved in a mixture of DCM (10 mL) and TFA (1.83 g, 16.09 mmol, 1.24 mL) and the resulting mixture was stirred at 25° C. for 15 hr. After that time, precipitate was filtered off, mother liquid was evaporated in vacuo to obtain crude product. Crude product was subjected by HPLC (column: XBridge BEH C18 5 μm 130 Å; mobile phase: 20-20-60% 0-1.3-5.3 min 5H₂O/MeOH/0.1% NH₄OH, flow rate: 30 mL/min (loading pump 4 mL/min MeOH) to obtain crude product. Crude product was by HPLC again (column: Chromatorex 18 SNB100-5T 100×19 mm, 5 um; mobile phase: 5-5-25% 0-1.3-5.3 min H₂O/MeCN/0.1% FA, flow rate: 30 mL/min (loading pump 4 mL/min MeCN) to obtain N-(4-amino-1H-pyrazolo[4,3-c]pyridin-7-yl)-2-oxo-2-[rac-(2S,5R)-5-methyl-4-(1-methylcyclopropanecarbonyl)-2-phenyl-piperazin-1-yl]acetamide (27.1 mg, 53.39 μmol, 6.64% yield, HCOOH).

¹H NMR (500 MHz, dmso) 6 0.31-0.85 (m, 4H), 0.92-1.08 (m, 3H), 1.11-1.41 (m, 3H), 2.64-3.16 (m, 2H), 3.53-3.81 (m, 1H), 3.91-4.19 (m, 1H), 4.26-4.67 (m, 1H), 4.72-5.77 (m, 1H), 6.64-6.99 (m, 2H), 7.07-7.40 (m, 5H), 7.45-7.73 (m, 1H), 8.12-8.17 (m, 1H), 9.63-10.78 (m, 1H), 12.69-13.43 (m, 1H).

LCMS(ESI): [M+H]⁺ m/z: calcd 462.2; found 462.2; Rt=2.175 min.

### Example 112. (Compound 170)N-(4-Amino-1H-pyrazolo[4,3-c]pyridin-7-yl)-2-oxo-2-[rac-(2S,5R)-4-cyclopropyl-5-methyl-2-phenyl-piperazin-1-yl]acetamide

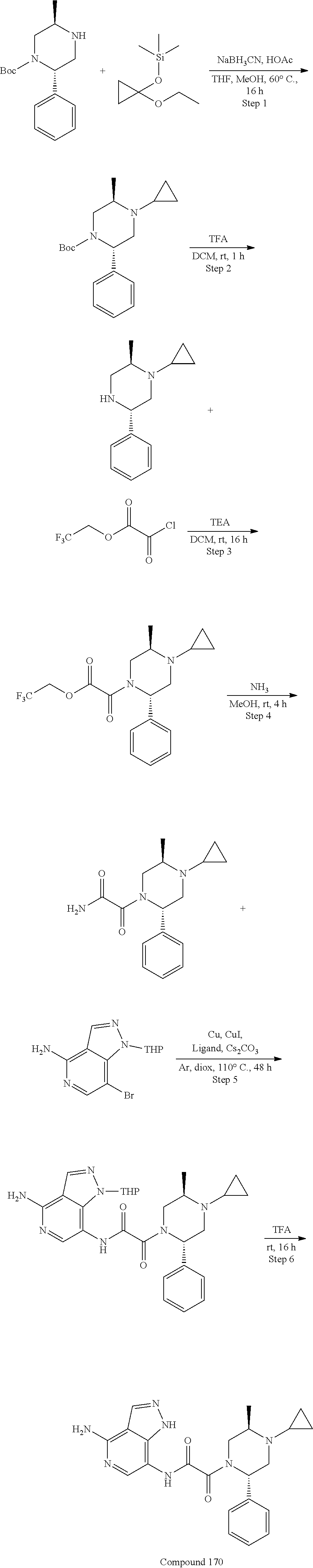

### Step 1: The Synthesis of tert-Butyl rac-(2S,5R)-4-cyclopropyl-5-methyl-2-phenyl-piperazine-1-carboxylate

tert-Butyl rac-(2S,5R)-5-methyl-2-phenyl-piperazine-1-carboxylate (962 mg, 3.48 mmol), (1-ethoxycyclopropoxy)-trimethyl-silane (1.21 g, 6.96 mmol, 1.40 mL) and Acetic acid (668.87 mg, 11.14 mmol, 637.63 L) were dissolved in a mixture of THF (24 mL) and MeOH (2.4 mL). Sodium cyanoborohydride (328.10 mg, 5.22 mmol) was added to the previous mixture and the resulting mixture was heated at 60° C. overnight. The reaction mixture was concentrated in vacuo and aq·K₂CO₃ solution (50 mL) was added thereto. The resulting mixture was extracted with MTBE (2*75 mL) and combined organic layers were dried over Na₂SO₄, filtered, and concentrated in vacuo to obtain tert-butyl rac-(2S,5R)-4-cyclopropyl-5-methyl-2-phenyl-piperazine-1-carboxylate (0.98 g, 3.10 mmol, 88.97% yield).

LCMS(ESI): [M+H]⁺ m/z: calcd 317.2; found 317.2; Rt=1.137 min.

### Step 2: The Synthesis of rac-(2R,5S)-1-Cyclopropyl-2-methyl-5-phenyl-piperazine

tert-Butyl rac-(2S,5R)-4-cyclopropyl-5-methyl-2-phenyl-piperazine-1-carboxylate (0.98 g, 3.10 mmol) was dissolved in DCM (5 mL) and TFA (5 mL) was added thereto. The resulting mixture was stirred for 1 hr. The reaction mixture was carefully poured into aq. K₂CO₃ solution (12 g in 45 mL of water) and the resulting mixture was extracted with DCM (2*50 mL). Combined organic layers were washed with water (45 mL), dried over Na₂SO₄, filtered and concentrated in vacuo to obtain rac-(2R,5S)-1-cyclopropyl-2-methyl-5-phenyl-piperazine (0.65 g, 3.00 mmol, 97.02% yield).

LCMS(ESI): [M+H]⁺ m/z: calcd 217.2; found 217.2; Rt=0.820 min.

### Step 3: The Synthesis of 2,2,2-Trifluoroethyl 2-oxo-2-[rac-(2S,5R)-4-cyclopropyl-5-methyl-2-phenyl-piperazin-1-yl]acetate

rac-(2R,5S)-1-Cyclopropyl-2-methyl-5-phenyl-piperazine (0.65 g, 3.00 mmol) and Triethylamine (349.66 mg, 3.46 mmol, 481.63 L) were dissolved in DCM (10 mL) and the resulting solution was cooled to −5° C. in an ice/methanol bath. 2,2,2-trifluoroethyl 2-chloro-2-oxo-acetate (629.67 mg, 3.31 mmol) was added dropwise to the previous solution and the resulting mixture was allowed to warm to room temperature and stirred overnight. Water (25 mL) was added to the reaction mixture and an organic layer was separated. The aqueous layer was extracted with DCM (25 mL) and combined organic layers were dried over Na₂SO₄, filtered and concentrated in vacuo to obtain 2,2,2-trifluoroethyl 2-oxo-2-[rac-(2S,5R)-4-cyclopropyl-5-methyl-2-phenyl-piperazin-1-yl]acetate (1.15 g, crude).

LCMS(ESI): [M+H]⁺ m/z: calcd 371.2; found 371.2; Rt=1.254 min.

### Step 4: The Synthesis of 2-Oxo-2-[rac-(2S,5R)-4-cyclopropyl-5-methyl-2-phenyl-piperazin-1-yl]acetamide

2,2,2-Trifluoroethyl 2-oxo-2-[rac-(2S,5R)-4-cyclopropyl-5-methyl-2-phenyl-piperazin-1-yl]acetate (1.15 g, 3.11 mmol) was dissolved in NH₃/MeOH (30 mL) and the resulting mixture was stirred for 4 hr. The reaction mixture was concentrated in vacuo and the residue was purified by HPLC to obtain 2-oxo-2-[rac-(2S,5R)-4-cyclopropyl-5-methyl-2-phenyl-piperazin-1-yl]acetamide (97.8 mg, 340.34 μmol, 10.96% yield) and 2-oxo-2-[rac-(2S,5R)-4-cyclopropyl-5-methyl-2-phenyl-piperazin-1-yl]acetamide (second fraction: 127.8 mg, 444.74 mol, 14.32% yield).

LCMS(ESI): [M+H]⁺ m/z: calcd 288.2; found 288.2; Rt=0.774 min.

### Step 5: The Synthesis of N-(4-Amino-2-tetrahydropyran-2-yl-pyrazolo[4,3-c]pyridin-7-yl)-2-oxo-2-[rac-(2S,5R)-4-cyclopropyl-5-methyl-2-phenyl-piperazin-1-yl]acetamide

To an 8 mL vial 2-oxo-2-[rac-(2S,5R)-4-cyclopropyl-5-methyl-2-phenyl-piperazin-1-yl]acetamide (127.8 mg, 444.74 mol), 7-bromo-2-tetrahydropyran-2-yl-pyrazolo[4,3-c]pyridin-4-amine (145.37 mg, 489.22 μmol), Copper (1.41 mg, 22.24 mol), Copper (I) iodide (42.35 mg, 222.37 mol, 7.54 μL), rac-(1R,2R)—N₁,N₂-dimethylcyclohexane-1,2-diamine (47.45 mg, 333.56 μmol), Cesium carbonate (289.81 mg, 889.49 mol) and Dioxane (3 mL) were charged and the resulting mixture was purged with argon for 5 min. The vial was sealed and heated at 110° C. over the weekend. The reaction mixture was cooled, diluted with MeOH (5 mL), and filtered. The filter cake was rinsed with MeOH (5 mL) and the filtrate was concentrated in vacuo. The residue was purified by HPLC (2-10 min, 20-70% 10H₂O/MeOH/0.1NH₄OH, flow 30 mL/min ((loading pump 4 mL MeOH); column: Chromatorex C18 SMB100-5T 100*19 mm, 5 microM) to obtain N-(4-amino-2-tetrahydropyran-2-yl-pyrazolo[4,3-c]pyridin-7-yl)-2-oxo-2-[rac-(2S,5R)-4-cyclopropyl-5-methyl-2-phenyl-piperazin-1-yl]acetamide (18.4 mg, 36.54 mol, 8.22% yield) and N-(4-amino-2-tetrahydropyran-2-yl-pyrazolo[4,3-c]pyridin-7-yl)-2-oxo-2-[rac-(2S,5R)-4-cyclopropyl-5-methyl-2-phenyl-piperazin-1-yl]acetamide (second fraction: 27.8 mg, 55.20 μmol, 12.41% yield).

LCMS(ESI): [M+2H]⁺ m/z: calcd 505.2; found 505.2; Rt=2.527 min.

### Step 6: The Synthesis of N-(4-Amino-1H-pyrazolo[4,3-c]pyridin-7-yl)-2-oxo-2-[rac-(2S,5R)-4-cyclopropyl-5-methyl-2-phenyl-piperazin-1-yl]acetamide (Compound 170)

N-(4-Amino-2-tetrahydropyran-2-yl-pyrazolo[4,3-c]pyridin-7-yl)-2-oxo-2-[rac-(2S,5R)-4-cyclopropyl-5-methyl-2-phenyl-piperazin-1-yl]acetamide (62.5 mg, 124.11 μmol) was dissolved in TFA (1.5 mL) and the resulting mixture was stirred overnight. The reaction mixture was concentrated in vacuo. The residue was combined with another batch and twice purified by HPLC (1st purification: 0-10-50%, 0-5 min H₂O/MeCN, flow: 30 mL/min (loading pump 4 mL/min H₂O); column: CHROMATOREX C18 100×19 mm 5 μm; 2nd purification: H₂O+0.1% FA/ACN, flow 30 mL/min, loading pump 4.0 mL/min H₂O) to obtain N-(4-amino-1H-pyrazolo[4,3-c]pyridin-7-yl)-2-oxo-2-[rac-(2S,5R)-4-cyclopropyl-5-methyl-2-phenyl-piperazin-1-yl]acetamide (6.7 mg, 14.39 mol, 11.60% yield, HCOOH).

¹H NMR (500 MHz, dmso) 6 0.06-0.56 (m, 4H), 0.95-1.16 (m, 3H), 1.77-1.99 (m, 1H), 2.69-3.16 (m, 4H), 3.63-4.10 (m, 1H), 4.82-5.66 (m, 1H), 6.24-6.85 (m, 2H), 6.88-7.46 (m, 5H), 7.46-7.77 (m, 1H), 8.10-8.93 (m, 2H), 9.53-10.69 (m, 1H), 12.57-13.43 (m, 1H).

LCMS(ESI): [M+H]⁺ m/z: calcd 420.2; found 420.2; Rt=1.011 min.

### Example 113. (Compound 167)N-(4-Amino-1H-pyrazolo[4,3-c]pyridin-7-yl)-2-oxo-2-[rac-(2S,5R)-5-methyl-4-(1-methylcyclopropyl)-2-phenyl-piperazin-1-yl]acetamide

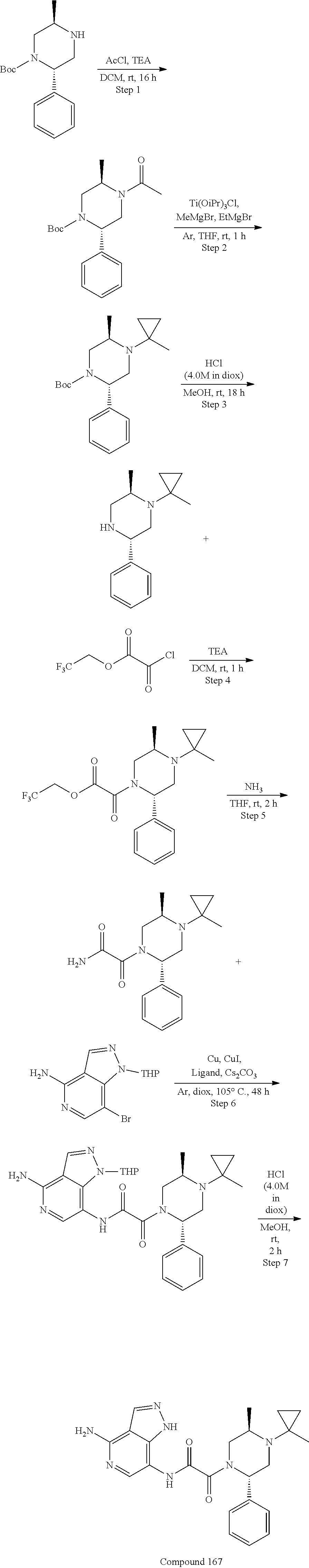

### Step 1: The Synthesis of tert-Butyl rac-(2S,5R)-4-acetyl-5-methyl-2-phenyl-piperazine-1-carboxylate

tert-Butyl rac-(2S,5R)-5-methyl-2-phenyl-piperazine-1-carboxylate (1 g, 3.62 mmol) and TEA (549.20 mg, 5.43 mmol, 756.48 L) were mixed together in DCM (24.22 mL) and the resulting solution was cooled to 5° C. in an ice bath Acetyl chloride (312.43 mg, 3.98 mmol, 241.45 L) was added dropwise to the previous solution and the resulting mixture was allowed to warm to rt and stirred overnight. Upon completion of the reaction, the mixture was washed with brine (2*20 mL), the organic layer was dried over Na₂SO₄, filtered, and concentrated in vacuo to afford tert-butyl rac-(2S,5R)-4-acetyl-5-methyl-2-phenyl-piperazine-1-carboxylate (1.3 g, crude) as a yellow gum.

LCMS(ESI): [M+H]⁺ m/z: calcd 319.2; found 319.2; Rt=3.035 min.

### Step 2: The Synthesis of tert-Butyl rac-(2S,5R)-5-methyl-4-(1-methylcyclopropyl)-2-phenyl-piperazine-1-carboxylate

tert-Butyl rac-(2S,5R)-4-acetyl-5-methyl-2-phenyl-piperazine-1-carboxylate (1.3 g, 3.27 mmol) is dissolved in dry THF (50 mL) under argon. To this was added a solution of MeTi(0iPr)₃ (prepared from Chlorotitanium Triisopropoxide (4.26 g, 16.33 mmol) and Methylmagnesium bromide (5.56 g, 16.33 mmol, 35% purity) over a period of 3 minutes followed by Ethylmagnesium chloride (2.90 g, 32.66 mmol) over a period of 7 minutes. The reaction mixture was allowed to stir at rt for an additional 30 minutes then carefully diluted with aq. NH₄Cl and MTBE. The mixture was stirred vigorously for 15 minutes, filtered, and extracted with MTBE (50 mL) and the combined organic layers were dried and evaporated. tert-Butyl rac-(2S,5R)-5-methyl-4-(1-methylcyclopropyl)-2-phenyl-piperazine-1-carboxylate (1.2 g, crude) was obtained as a yellow gum.

LCMS(ESI): [M+H]⁺ m/z: calcd 331.2; found 331.4; Rt=2.897 min.

### Step 3: The Synthesis of rac-(2R,5S)-2-Methyl-1-(1-methylcyclopropyl)-5-phenyl-piperazine

To a solution of tert-butyl rac-(2S,5R)-5-methyl-4-(1-methylcyclopropyl)-2-phenyl-piperazine-1-carboxylate (1.2 g, 2.91 mmol) in MeOH (15 mL) was added Hydrogen chloride solution 4.0 μM in dioxane (105.92 mg, 2.91 mmol, 132.40 L) at 21° C. The resulting mixture was left to stir for 18 hr. The resulting mixture was evaporated to dryness and used in the next step without further purification. rac-(2R,5S)-2-methyl-1-(1-methylcyclopropyl)-5-phenyl-piperazine (1.2 g, crude, 2HCl) was obtained as a yellow gum.

LCMS(ESI): [M+H]⁺ m/z: calcd 231.2; found 231.2; Rt=2.013 min.

### Step 4: The Synthesis of 2,2,2-Trifluoroethyl 2-oxo-2-[rac-(2S,5R)-5-methyl-4-(1-methylcyclopropyl)-2-phenyl-piperazin-1-yl]acetate

To a solution of rac-(2R,5S)-2-methyl-1-(1-methylcyclopropyl)-5-phenyl-piperazine (1.2 g, 2.60 mmol) and TEA (1.05 g, 10.42 mmol, 1.45 mL) in DCM (25 mL) was added 2,2,2-trifluoroethyl carbonochloridate (465.59 mg, 2.87 mmol) at rt. After stirring at rt for 1 hr the resulting mixture was washed with water, dried and evaporated to dryness to give 2,2,2-trifluoroethyl 2-oxo-2-[rac-(2S,5R)-5-methyl-4-(1-methylcyclopropyl)-2-phenyl-piperazin-1-yl]acetate (0.8 g, crude) as a yellow gum and was used in the next step without further purification.

LCMS(ESI): [M+H]⁺ m/z: calcd 385.2; found 385.2; Rt=3.488 min.

### Step 5: The Synthesis of 2-Oxo-2-[rac-(2S,5R)-5-methyl-4-(1-methylcyclopropyl)-2-phenyl-piperazin-1-yl]acetamide

Gaseous ammonia (1.77 g, 104.06 mmol) was vigorously bubbled at 25° C. for 0.5 hr through a solution of 2,2,2-trifluoroethyl 2-oxo-2-[rac-(2S,5R)-5-methyl-4-(1-methylcyclopropyl)-2-phenyl-piperazin-1-yl]acetate (800 mg, 2.08 mmol) in THF (40 mL). The resulting mixture was then additionally stirred at 25° C. for 1 hr, and then concentrated in vacuo. The residue was purified by reverse phase HPLC (column: XBridge BEH C18 100×19 mm, 5 um; mobile phase: 40-40-80% 0-1-6 min H₂O/MeOH/0.1% NH₄OH, flow rate: 30 mL/min (loading pump 4 mL/min methanol)) to afford 2-oxo-2-[rac-(2S,5R)-5-methyl-4-(1-methylcyclopropyl)-2-phenyl-piperazin-1-yl]acetamide (100 mg, 331.80 mol, 15.94% yield) as orange gum.

LCMS(ESI): [M+H]⁺ m/z: calcd 302.2; found 302.2; Rt=1.677 min.

### Step 6: The Synthesis of N-(4-Amino-2-tetrahydropyran-2-yl-pyrazolo[4,3-c]pyridin-7-yl)-2-oxo-2-[rac-(2S,5R)-5-methyl-4-(1-methylcyclopropyl)-2-phenyl-piperazin-1-yl]acetamide

A mixture of 2-oxo-2-[rac-(2S,5R)-5-methyl-4-(1-methylcyclopropyl)-2-phenyl-piperazin-1-yl]acetamide (100 mg, 331.80 μmol), 7-bromo-2-tetrahydropyran-2-yl-pyrazolo[4,3-c]pyridin-4-amine (197.19 mg, 663.61 mol), copper (2.75 mg, 43.31 mol), Copper (I) iodide (120 mg, 630.09 μmol, 21.35 μL), caesium carbonate (194.60 mg, 597.25 mol) and rac-(1R,2R)—N₁,N₂-dimethylcyclohexane-1,2-diamine (120 mg, 843.64 mol) in 1,4-dioxane (6.00 mL) was stirred in a sealed vial under argon at 105° C. for 42 hr. The resulting mixture was cooled down and filtered. The filtercake was washed successively with THF (2*5 mL) and dichloromethane (3*5 mL). The combined filtrate was concentrated in vacuo to afford N-(4-amino-2-tetrahydropyran-2-yl-pyrazolo[4,3-c]pyridin-7-yl)-2-oxo-2-[rac-(2S,5R)-5-methyl-4-(1-methylcyclopropyl)-2-phenyl-piperazin-1-yl]acetamide (600 mg, crude) as brown gum, which was used directly in the next step.

LCMS(ESI): [M+H]⁺ m/z: calcd 518.2; found 518.2; Rt=2.459 min.

### Step 7: The Synthesis of N-(4-Amino-1H-pyrazolo[4,3-c]pyridin-7-yl)-2-oxo-2-[rac-(2S,5R)-5-methyl-4-(1-methylcyclopropyl)-2-phenyl-piperazin-1-yl]acetamide (Compound 167)

Hydrogen chloride solution 4.0 M in dioxane (4.20 g, 16.01 mmol, 4 mL, 13.9% purity) was added to a stirred solution of crude from previous step N-(4-amino-2-tetrahydropyran-2-yl-pyrazolo[4,3-c]pyridin-7-yl)-2-oxo-2-[rac-(2S,5R)-5-methyl-4-(1-methylcyclopropyl)-2-phenyl-piperazin-1-yl]acetamide (600 mg, 1.16 mmol) in methanol (4 mL) at 25° C. The resulting solution was stirred at 25° C. for 1.5 hr, then concentrated to dryness in vacuo and the residue was submitted to reverse phase HPLC (column: Chromatorex 18 SMB100-5T 100×19 mm 5 um; mobile phase: 0-0-20% 0-2-4 min H₂O/Acetonitrile/0.2% FA; flow: 40 mL/min (loading pump 4 mL/min acetonitrile)) to afford Compound 167 N-(4-amino-1H-pyrazolo[4,3-c]pyridin-7-yl)-2-oxo-2-[rac-(2S,5R)-5-methyl-4-(1-methylcyclopropyl)-2-phenyl-piperazin-1-yl]acetamide (33 mg, 68.82 mol, 5.94% yield, HCOOH) as beige solid.

¹H NMR (500 MHz, dmso) 6 0.14-0.56 (m, 4H), 0.73-1.24 (m, 6H), 3.01-3.20 (m, 3H), 3.51-4.03 (m, 2H), 4.74-5.45 (m, 1H), 6.30-7.78 (m, 8H), 8.20-8.43 (m, 1H), 9.59-10.79 (m, 1H), 12.74-13.46 (m, 1H).

LCMS(ESI): [M+2H]⁺ m/z: calcd 435.2; found 435.2; Rt=1.738 min.

### Example 114. (Compound 162)N-(4-Amino-1H-pyrazolo[4,3-c]pyridin-7-yl)-2-oxo-2-[rac-(2S,5R)-4-ethyl-5-methyl-2-phenyl-piperazin-1-yl]acetamide

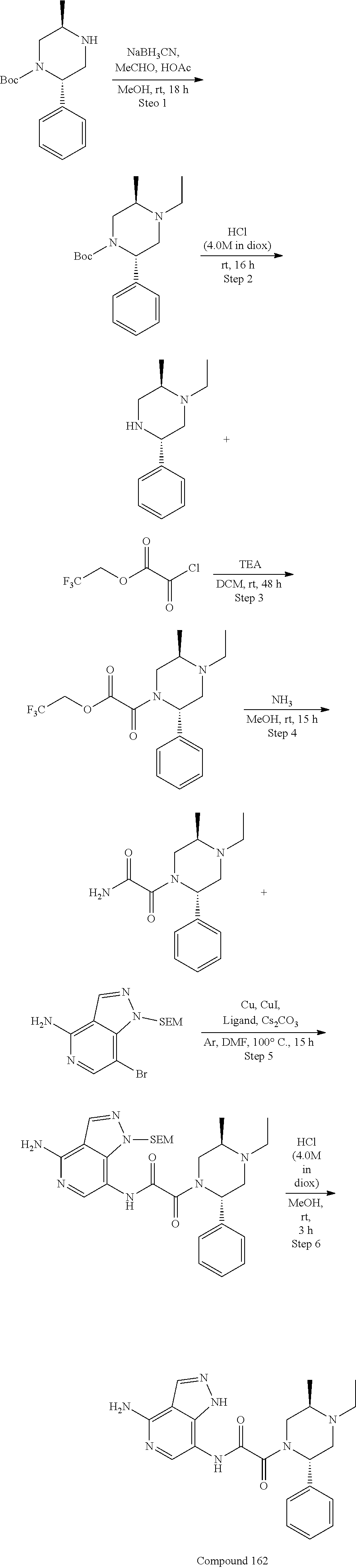

### Step 1: The Synthesis of tert-Butyl rac-(2S,5R)-4-ethyl-5-methyl-2-phenyl-piperazine-1-carboxylate

Acetaldehyde (334.73 mg, 7.60 mmol) and Acetic acid (304.20 mg, 5.07 mmol, 289.99 μL) were added to a stirred solution of tert-butyl rac-(2S,5R)-5-methyl-2-phenyl-piperazine-1-carboxylate (0.7 g, 2.53 mmol) in MeOH (30 mL) at 25° C. The resulting mixture was stirred at 25° C. for 0.5 hr, then Sodium cyanoborohydride (318.33 mg, 5.07 mmol) was added in one portion at 25° C. The reaction mixture was stirred at 25° C. for 16 hr, and then concentrated in vacuo. The residue was diluted with 10% aqueous sodium hydroxide solution (20 mL) and extracted with dichloromethane (2*20 mL). The combined organic extracts were dried over sodium sulphate and concentrated in vacuo to afford tert-butyl rac-(2S,5R)-4-ethyl-5-methyl-2-phenyl-piperazine-1-carboxylate (0.9 g, crude).

LCMS(ESI): [M+H]⁺ m/z: calcd 305.2; found 305.2; Rt=0.841 min.

### Step 2: The Synthesis of rac-(2R,5S)-1-Ethyl-2-methyl-5-phenyl-piperazine

tert-Butyl rac-(2S,5R)-4-ethyl-5-methyl-2-phenyl-piperazine-1-carboxylate (0.9 g, 2.96 mmol) was dissolved in Diox/Hcl (20 mL), the resulting mixture was left at 26° C. for 18 hr. Upon completion, the solvent was concentrated to dryness, water was added to the residue (20 mL) and basified with K₂CO₃ to alkaline pH, aqueous phase was extracted with DCM (3*15 mL), combined organic layers were dried over Na₂SO₄, filtered and concentrated in vacuo to obtain rac-(2R,5S)-1-ethyl-2-methyl-5-phenyl-piperazine (0.6 g, crude).

LCMS(ESI): [M+H]⁺ m/z: calcd 205.2; found 205.2; Rt=0.445 min.

### Step 3: The Synthesis of 2,2,2-Trifluoroethyl 2-oxo-2-[rac-(2S,5R)-4-ethyl-5-methyl-2-phenyl-piperazin-1-yl]acetate

rac-(2R,5S)-1-Ethyl-2-methyl-5-phenyl-piperazine (0.6 g, 2.94 mmol) and TEA (445.75 mg, 4.41 mmol, 613.98 L) were mixed together in DCM (20 mL) and the resulting solution was cooled to 5° C. in an ice bath. 2,2,2-Trifluoroethyl 2-chloro-2-oxo-acetate (615.40 mg, 3.23 mmol) was added dropwise to the previous solution and the resulting mixture was allowed to warm to rt and stirred for 48 hr. Upon completion of the reaction, the mixture was washed with brine (2*20 mL), organic layer was dried over Na₂SO₄, filtered and concentrated in vacuo to affording 2,2,2-trifluoroethyl 2-oxo-2-[rac-(2S,5R)-4-ethyl-5-methyl-2-phenyl-piperazin-1-yl]acetate (0.8 g, crude).

LCMS(ESI): [M+H]⁺ m/z: calcd 359.0; found 359.0; Rt=0.697 min.

### Step 4: The Synthesis of 2-Oxo-2-[rac-(2S,5R)-4-ethyl-5-methyl-2-phenyl-piperazin-1-yl]acetamide

2,2,2-Trifluoroethyl 2-oxo-2-[rac-(2S,5R)-4-ethyl-5-methyl-2-phenyl-piperazin-1-yl]acetate (0.8 g, 2.23 mmol) was dissolved in NH₃/MeOH (25 mL). The resulting clear solution was left for 15 hr at 25° C. Upon completion of the reaction the resulting mixture was concentrated to dryness to obtain 2-oxo-2-[rac-(2S,5R)-4-ethyl-5-methyl-2-phenyl-piperazin-1-yl]acetamide (550 mg, crude).

LCMS(ESI): [M+H]⁺ m/z: calcd 276.2; found 276.2; Rt=0.402 min.

### Step 5: The Synthesis of N-[4-Amino-2-(2-trimethylsilylethoxymethyl)pyrazolo[4,3-c]pyridin-7-yl]-2-oxo-2-[rac-(2S,5R)-4-ethyl-5-methyl-2-phenyl-piperazin-1-yl]acetamide

A mixture of 2-oxo-2-[rac-(2S,5R)-4-ethyl-5-methyl-2-phenyl-piperazin-1-yl]acetamide (0.3 g, 1.09 mmol), 7-bromo-2-(2-trimethylsilylethoxymethyl)pyrazolo[4,3-c]pyridin-4-amine (374.03 mg, 1.09 mmol), Cu (34.62 mg, 544.77 mol), Cesium carbonate (709.99 mg, 2.18 mmol), CuI (207.50 mg, 1.09 mmol, 36.92 L) and (1S,2S)—N,N′-Bis-methyl-1,2-cyclohexane-diamine (232.46 mg, 1.63 mmol, 257.72 L) in DMF (3.83 mL) was stirred in a sealed vial under argon at 100° C. for 15 hr. After that time, solvent was evaporated to dryness to afford crude product, then crude product was subjected by HPLC (column: XBridge C18 100×19 mm, 5 um; mobile phase: 50-100% 0-5 min H₂O/MeOH/0.1% NH₄OH, flow rate: 30 mL/min (loading pump 4 mL/min MeOH) to obtain N-[4-amino-2-(2-trimethylsilylethoxymethyl)pyrazolo[4,3-c]pyridin-7-yl]-2-oxo-2-[rac-(2S,5R)-4-ethyl-5-methyl-2-phenyl-piperazin-1-yl]acetamide (83.5 mg, crude).

LCMS(ESI): [M+2H]⁺ m/z: calcd 539.2; found 539.2; Rt=0.751 min.

### Step 6: The Synthesis of N-(4-Amino-1H-pyrazolo[4,3-c]pyridin-7-yl)-2-oxo-2-[rac-(2S,5R)-4-ethyl-5-methyl-2-phenyl-piperazin-1-yl]acetamide (Compound 162)

N-[4-Amino-2-(2-trimethylsilylethoxymethyl)pyrazolo[4,3-c]pyridin-7-yl]-2-oxo-2-[rac-(2S,5R)-4-ethyl-5-methyl-2-phenyl-piperazin-1-yl]acetamide (83.5 mg, 155.28 μmol) was dissolved in a mixture of Diox/HCl (1.5 mL) and MeOH (1.5 mL), the resulting mixture was stirred at 25° C. for 3 hr. After that time, solvent was evaporated to dryness to afford crude product, then crude product was subjected by HPLC (column: Chromatorex 18 SMB100-5T 100×19 mm, 5 um; mobile phase: 0-O-10% 0-1.5-5 min H₂O/MeCN/0.1% NH₄OH, flow rate: 30 mL/min (loading pump 4 mL/min MeCN), affording N-(4-amino-1H-pyrazolo[4,3-c]pyridin-7-yl)-2-oxo-2-[rac-(2S,5R)-4-ethyl-5-methyl-2-phenyl-piperazin-1-yl]acetamide (15.8 mg, 34.84 mol, 22.44% yield, HCOO—).

¹H NMR (600 MHz, dmso) 6-0.05-1.08 (m, 6H), 2.38-2.44 (m, 2H), 2.73-2.88 (m, 1H), 2.89-3.03 (m, 1H), 3.05-3.12 (m, 1H), 3.18-3.20 (m, 1H), 3.81-4.17 (m, 1H), 4.87-5.65 (m, 1H), 6.41-6.94 (m, 2H), 6.96-7.48 (m, 3H), 7.48-7.73 (m, 2H), 8.07-8.24 (m, 2H), 9.63-10.53 (m, 1H), 12.50-13.36 (m, 1H).

LCMS(ESI): [M+2H]⁺ m/z: calcd 409.2; found 409.2; Rt=1.208 min.

### Example 115. (Compound 166)N-(7-Amino-1-methyl-pyrazolo[3,4-c]pyridin-4-yl)-2-oxo-2-[(2S,5R)-5-methyl-2-phenyl-4-[1-(trifluoromethyl)cyclopropanecarbonyl]piperazin-1-yl]acetamide

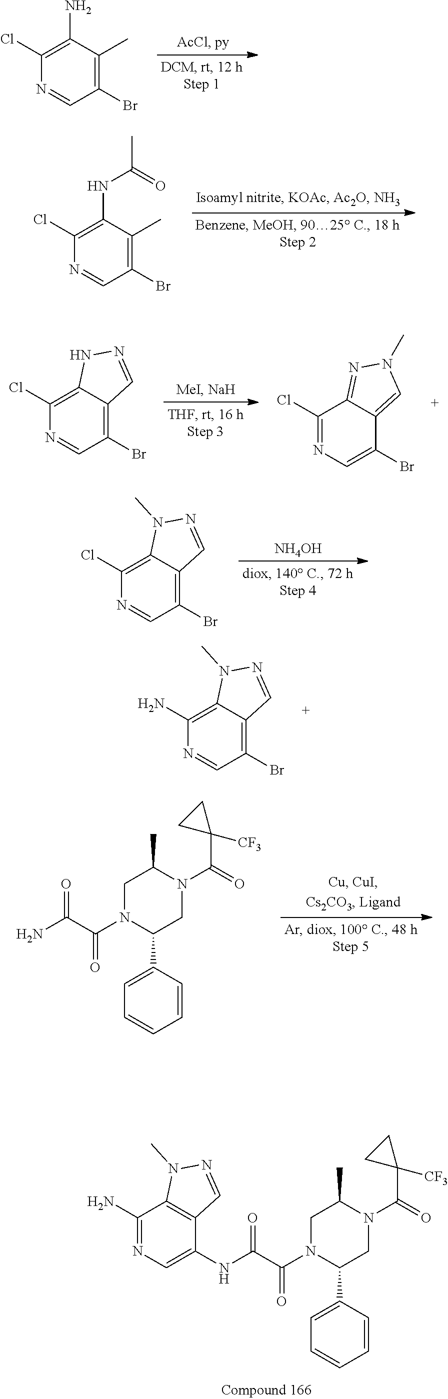

### Step 5: The Synthesis of N-(7-Amino-1-methyl-pyrazolo[3,4-c]pyridin-4-yl)-2-oxo-2-[(2S,5R)-5-methyl-2-phenyl-4-[1-(trifluoromethyl)cyclopropanecarbonyl]piperazin-1-yl]acetamide (Compound 166)

2-Oxo-2-[(2S,5R)-5-methyl-2-phenyl-4-[1-(trifluoromethyl)cyclopropanecarbonyl]piperazin-1-yl]acetamide (135.07 mg, 352.33 mol), 4-bromo-1-methyl-pyrazolo[3,4-c]pyridin-7-amine (0.08 g, 352.33 mol), Copper (1.12 mg, 17.62 mol), Copper (I) iodide (33.55 mg, 176.16 mol, 5.97 μL), (1R,2R)—N₁,N₂-dimethylcyclohexane-1,2-diamine (37.59 mg, 264.25 mol) and Cesium carbonate (229.59 mg, 704.66 mol) were mixed in Dioxane (4 mL). The reaction mixture was purged with argon for 5 min. Vials were sealed and heated at 100° C. for 48 hr. The reaction mixture was cooled and filtered. The filter cake was rinsed with MeOH (10 mL) and the filtrate was concentrated in vacuo. Resulting crude material was purified by HPLC (8-15-40% 0-2-10H₂O/ACN/0.1NH₄OH, flow 30 mL/min ((loading pump; 4 mL ACN); column: XBridge BEH C18 100*19 mm,5 microM) to obtain N-(7-amino-1-methyl-pyrazolo[3,4-c]pyridin-4-yl)-2-oxo-2-[(2S,5R)-5-methyl-2-phenyl-4-[1-(trifluoromethyl)cyclopropanecarbonyl]piperazin-1-yl]acetamide (3.6 mg, 6.80 mol, 1.93% yield).

¹H NMR (600 MHz, dmso) 6 0.91-1.02 (m, 1H), 1.14-1.38 (m, 6H), 2.60-3.27 (m, 2H), 3.57-4.22 (m, 2H), 4.22-4.30 (m, 3H), 4.44-5.92 (m, 2H), 6.11-6.29 (m, 2H), 7.19-7.38 (m, 5H), 7.69-8.02 (m, 2H), 10.63-11.01 (m, 1H).

LCMS(ESI): [M+H]⁺ m/z: calcd 530.2; found 530.2; Rt=0.862 min.

### Example 116. (Compound 169)N-(7-Amino-1-methyl-pyrazolo[3,4-c]pyridin-4-yl)-2-oxo-2-[(2S,5R)-5-methyl-4-(1-methylcyclopropanecarbonyl)-2-phenyl-piperazin-1-yl]acetamide

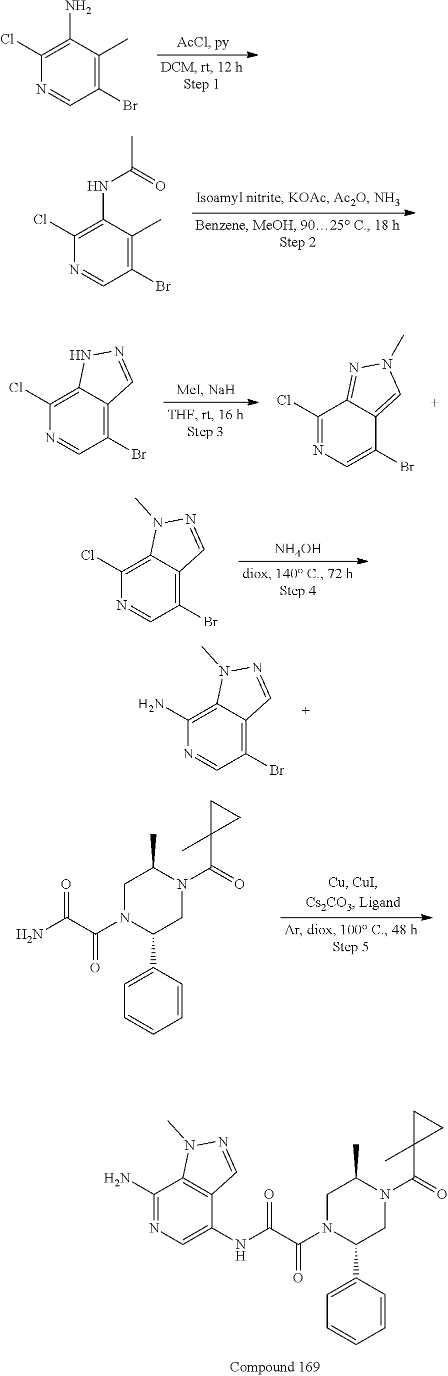

### Step 5: The Synthesis of N-(7-Amino-1-methyl-pyrazolo[3,4-c]pyridin-4-yl)-2-oxo-2-[(2S,5R)-5-methyl-4-(1-methylcyclopropanecarbonyl)-2-phenyl-piperazin-1-yl]acetamide

(Compound 169)

2-Oxo-2-[(2S,5R)-5-methyl-4-(1-methylcyclopropanecarbonyl)-2-phenyl-piperazin-1-yl]acetamide (116.05 mg, 352.33 mol), 4-bromo-1-methyl-pyrazolo[3,4-c]pyridin-7-amine (0.08 g, 352.33 μmol), Copper (1.12 mg, 17.62 mol), Copper (I) iodide (33.55 mg, 176.16 μmol, 5.97 μL), (1R,2R)—N₁,N₂-dimethylcyclohexane-1,2-diamine (37.59 mg, 264.25 mol) and Cesium carbonate (229.59 mg, 704.66 mol) were mixed in Dioxane (4 mL). The reaction mixture was purged with argon for 5 min. Vials were sealed and heated at 100° C. for 48 hr. The reaction mixture was cooled and filtered. The filter cake was rinsed with MeOH (10 mL) and the filtrate was concentrated in vacuo. Resulting crude material was purified by HPLC (3-10-65% 0-2-10H₂O/MeOH/O.1FA, flow 30 mL/min ((loading pump 4 mL MeOH); column: XBridge BEH C18 100*19 mm,5 mkm) to obtain N-(7-amino-1-methyl-pyrazolo[3,4-c]pyridin-4-yl)-2-oxo-2-[(2S,5R)-5-methyl-4-(1-methylcyclopropanecarbonyl)-2-phenyl-piperazin-1-yl]acetamide (12.3 mg, 25.87 mol, 7.34% yield).

1H NMR (600 MHz, dmso) 6 0.30-0.88 (m, 4H), 1.04 (s, 3H), 1.12-1.40 (m, 3H), 2.65-3.23 (m, 2H), 3.37-4.14 (m, 2H), 4.14-4.28 (m, 3H), 4.27-5.90 (m, 2H), 6.02-6.60 (m, 2H), 7.24-7.40 (m, 5H), 7.72-8.38 (m, 2H), 10.49-11.13 (m, 1H).

LCMS(ESI): [M+H]⁺ m/z: calcd 476.2; found 476.2; Rt=0.985 min.

### Example 117. (Compound 165)N-(7-Amino-1-methyl-pyrazolo[3,4-c]pyridin-4-yl)-2-oxo-2-[(2S,5R)-5-methyl-4-(2-methylpropanoyl)-2-phenyl-piperazin-1-yl]acetamide

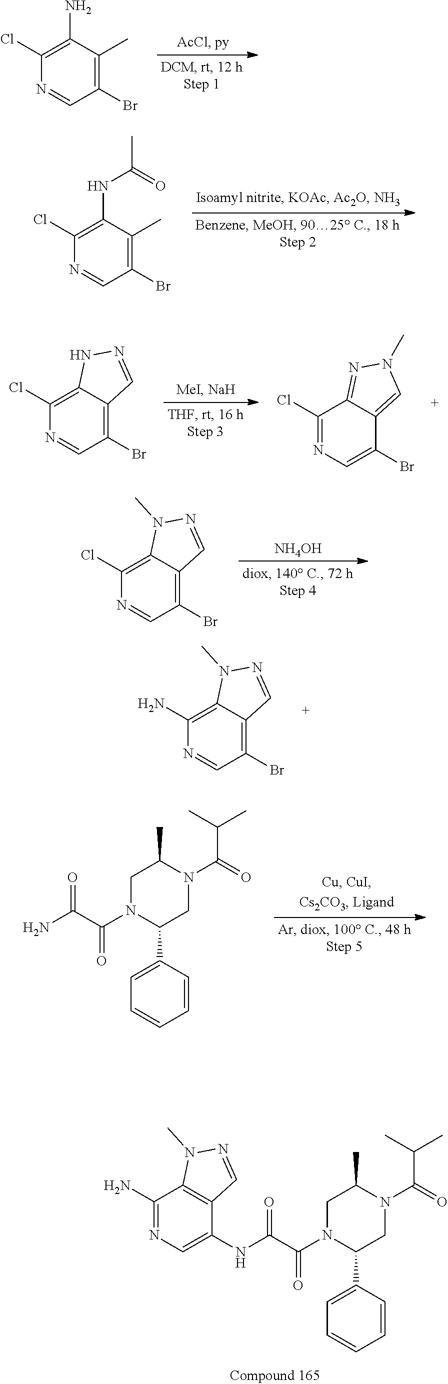

### Step 5: The Synthesis of N-(7-Amino-1-methyl-pyrazolo[3,4-c]pyridin-4-yl)-2-oxo-2-[(2S,5R)-5-methyl-4-(2-methylpropanoyl)-2-phenyl-piperazin-1-yl]acetamide (Compound 165)

2-Oxo-2-[(2S,5R)-5-methyl-4-(2-methylpropanoyl)-2-phenyl-piperazin-1-yl]acetamide (48 mg, 151.24 mol), 4-bromo-1-methyl-pyrazolo[3,4-c]pyridin-7-amine (34.34 mg, 151.24 μmol), Copper (480.56 g, 7.56 mol), Copper (I) iodide (14.40 mg, 75.62 μmol, 2.56 μL), (1R,2R)—N₁,N₂-dimethylcyclohexane-1,2-diamine (16.13 mg, 113.43 mol) and Cesium carbonate (98.55 mg, 302.47 mol) were mixed in Dioxane (2 mL). The reaction mixture was spurged with argon for 5 min. Vials were sealed and heated at 100° C. for 48 hr. The reaction mixture was cooled and filtered. The filter cake was rinsed with MeOH (10 ml) and the filtrate was concentrated in vacuo. Resulting crude material was purified by HPLC (0-2-9 min 0-5-60% MeOH/H₂O+FA, 30 mL/min (loading pump 4 mL MeOH); column: Chromarorex C18 SMB100-5T 100*19 mm, 5 microM) to obtain N-(7-amino-1-methyl-pyrazolo[3,4-c]pyridin-4-yl)-2-oxo-2-[(2S,5R)-5-methyl-4-(2-methylpropanoyl)-2-phenyl-piperazin-1-yl]acetamide (4.5 mg, 9.71 mol, 6.42% yield) and N-(7-amino-1-methyl-pyrazolo[3,4-c]pyridin-4-yl)-2-oxo-2-[(2S,5R)-5-methyl-4-(2-methylpropanoyl)-2-phenyl-piperazin-1-yl]acetamide (1.5 mg, 3.24 mol, 2.14% yield).

¹H NMR (600 MHz, dmso) 6 0.75-0.88 (m, 3H), 0.93-1.03 (m, 3H), 1.14-1.30 (m, 3H), 2.72-2.80 (m, 1H), 3.15-3.18 (m, 1H), 3.54-3.87 (m, 2H), 4.06-4.27 (m, 4H), 4.46-5.06 (m, 1H), 5.20-5.83 (m, 1H), 6.14-6.30 (m, 2H), 7.21-7.31 (m, 2H), 7.34-7.40 (m, 3H), 7.59-7.83 (m, 1H), 7.85-7.96 (m, 1H), 10.47-10.95 (m, 1H).

LCMS(ESI): [M+H]⁺ m/z: calcd 464.2; found 464.2; Rt=0.792 min.

### Example 118. (Compound 168)N-(7-Amino-1-methyl-pyrazolo[3,4-c]pyridin-4-yl)-2-oxo-2-[(2S,5R)-4-acetyl-5-methyl-2-phenyl-piperazin-1-yl]acetamide

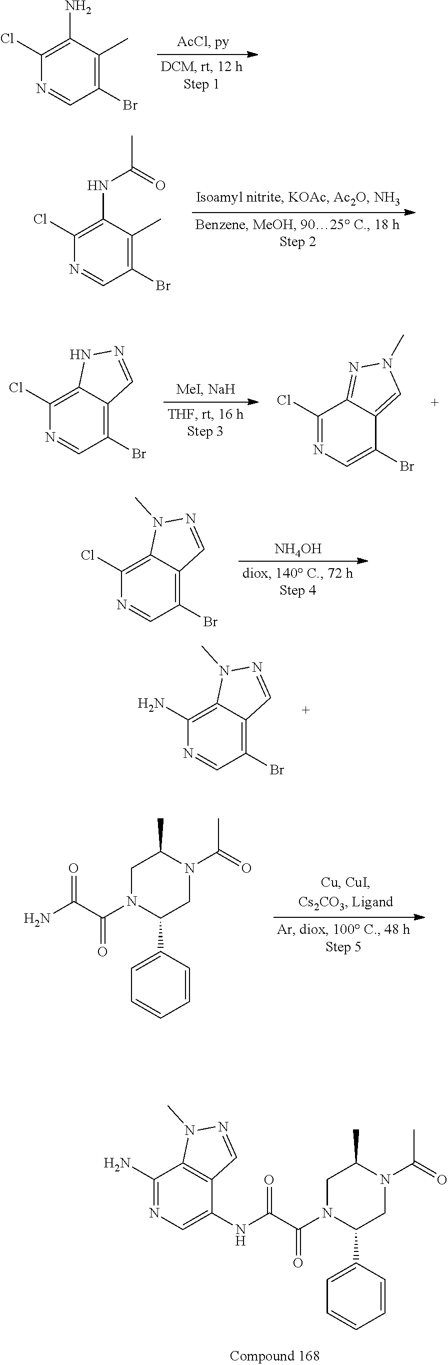

### Step 5: The Synthesis of N-(7-Amino-1-methyl-pyrazolo[3,4-c]pyridin-4-yl)-2-oxo-2-[(2S,5R)-4-acetyl-5-methyl-2-phenyl-piperazin-1-yl]acetamide (Compound 168)

2-Oxo-2-[(2S,5R)-4-acetyl-5-methyl-2-phenyl-piperazin-1-yl]acetamide (150 mg, 518.44 μmol), 4-bromo-1-methyl-pyrazolo[3,4-c]pyridin-7-amine (117.72 mg, 518.44 mol), Copper (1.65 mg, 25.92 mol), Copper (I) iodide (49.37 mg, 259.22 mol, 8.78 μL), (1R,2R)—N₁,N₂-dimethylcyclohexane-1,2-diamine (55.31 mg, 388.83 μmol) and Cesium carbonate (337.84 mg, 1.04 mmol) were mixed in Dioxane (4 mL). The reaction mixture was purged with argon for 5 min. Vials were sealed and heated at 95° C. for 48 hr. The reaction mixture was cooled and filtered. The filter cake was rinsed with MeOH (10 mL) and the filtrate was concentrated in vacuo. Resulting crude material was purified by HPLC (0-2-9 min 8-15-35% MeOH/H₂O+NH₄OH; flow 30 mL/min (loading pump 4 mL MeOH); column: XBridge BEH C18 100*19 mm, 5 microM) to obtain N-(7-amino-1-methyl-pyrazolo[3,4-c]pyridin-4-yl)-2-oxo-2-[(2S,5R)-4-acetyl-5-methyl-2-phenyl-piperazin-1-yl]acetamide (9.6 mg, 22.04 mol, 4.25% yield).

LCMS(ESI): [M+H]⁺ m/z: calcd 436.2; found 436.2; Rt=0.896 min.

### The Synthesis of N-(7-Amino-1-methyl-pyrazolo[3,4-c]pyridin-4-yl)-2-oxo-2-[(2S,5R)-4,5-dimethyl-2-phenyl-piperazin-1-yl]acetamide (Compound 164)

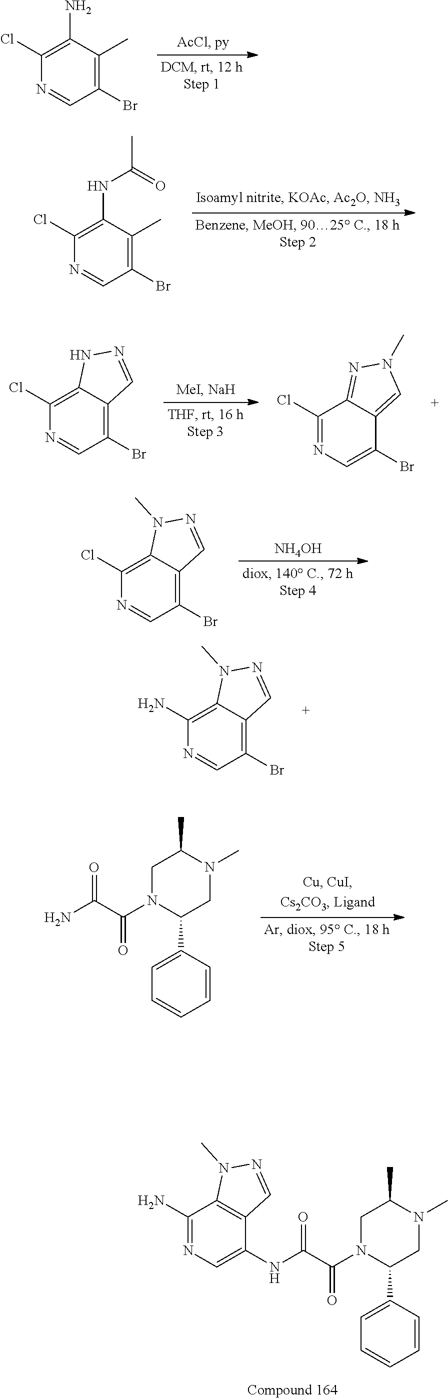

### Step 5: The Synthesis of N-(7-Amino-1-methyl-pyrazolo[3,4-c]pyridin-4-yl)-2-oxo-2-[(2S,5R)-4,5-dimethyl-2-phenyl-piperazin-1-yl]acetamide (Compound 164)

2-Oxo-2-[(2S,5R)-4,5-dimethyl-2-phenyl-piperazin-1-yl]acetamide (172.63 mg, 660.62 mol), 4-bromo-1-methyl-pyrazolo[3,4-c]pyridin-7-amine (150 mg, 660.62 mol), Copper (2.10 mg, 33.03 mol), Copper (I) iodide (62.91 mg, 330.31 mol, 11.19 μL), (1R,2R)—N₁,N₂-dimethylcyclohexane-1,2-diamine (70.47 mg, 495.46 μmol) and Cesium carbonate (322.86 mg, 990.92 μmol) were mixed in Dioxane (4 mL). The reaction mixture was purged with argon for 5 min. Vials were sealed and heated at 100° C. for 48 hr. The reaction mixture was cooled and filtered. The filter cake was rinsed with MeOH (10 mL) and the filtrate was concentrated in vacuo. Resulting crude material was purified by HPLC (0-2-9 min 13-20-40% MeOH/H₂O+NH₄OH; flow 30 mL/min (loading pump 4 mL MeOH); column: XBridge BEH C18 100*19 mm, 5 microM) to obtain N-(7-amino-1-methyl-pyrazolo[3,4-c]pyridin-4-yl)-2-oxo-2-[(2S,5R)-4,5-dimethyl-2-phenyl-piperazin-1-yl]acetamide (18.3 mg, 44.91 mol, 6.80% yield).

1H NMR (600 MHz, dmso) 6 0.95-1.01 (m, 3H), 2.21-2.26 (m, 3H), 2.84-2.95 (m, 2H), 3.02-3.11 (m, 1H), 3.34-4.06 (m, 2H), 4.22-4.29 (m, 3H), 5.05-5.54 (m, 1H), 6.18-6.26 (m, 2H), 7.23-7.29 (m, 1H), 7.31-7.39 (m, 2H), 7.42-7.53 (m, 2H), 7.75-7.98 (m, 2H), 10.34 (br s, 1H).

LCMS(ESI): [M+H]⁺ m/z: calcd 408.2; found 408.2; Rt=0.473 min.

### Example 119. Cellular assay—SDMA in-cell western protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 10% C₀₂ tissue culture incubator. The SAM-cooperative PRMT5 inhibitor, GSK3326595, was sourced from SelleckChem and maintained as a 10 mM DMSO stock. All test compounds are maintained as 10 mM DMSO stocks

On Day 0, MTAP-intact orMTAP-deleted cells are seeded in a 384-well plate, and incubated in a humidified, 5% CO₂ tissue culture incubator for 16-24 hours. On Day 1, the test compounds are dispensed to wells at defined concentrations using a Tecan D300e digital dispenser (n=4), and the volume of DMSO is normalized to highest class volume. Each plate includes wells dosed with defined concentrations of GSK33226595 as a plate control. The compounds are incubated with cells for 24 hours in a humidified, 5% CO₂ tissue culture incubator.

On Day 2, the compound-treated cells are fixed with a final concentration of 4% formaldehyde. The cells are then washed/permeabilized with 1×PBS+0.1% Triton X⁻100, and then blocked with 5% goat serum/1×TBS. The fixed cells are then incubated overnight at 4° C. with a primary SDMA antibody cocktail (Cell Signaling 13222).

On Day 3, the cells are washed with 1×PBS+0.1% Triton X⁻100, and then incubated at room temperature for 1 hour with a NIR fluorescent secondary antibody cocktail that also contains DRAQ5 (LiCor 926-32211 and VWR 10761-508). The cells are washed with 1×PBS+0.1% Triton X-100, and then washed again with ddH₂O. The plates are then imaged using a NIR fluorescent imager (LiCor Odyssey).

For data analysis, the SDMA signal is normalized to the DRAQ5 signal. Assay background is determined by the signal from wells treated with 1 μM GSK3326595, and subtracted from every well. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC₅₀ fitting procedure in Dotmatics Studies 5.4 as part of a customized data analysis protocol.

The data obtained in this experiment is presented in Table 1, columns 4-6.

### Example 120. Viability assay protocol

A HAP1 MTAP-isogenic cell line pair was acquired from Horizon Discovery (HZGHC004894c005) and maintained in DMEM (ThermoFisher 11965)+10% FBS (Gemini 100-500) in a humidified, 5 or 10% CO₂ tissue culture incubator. All test compounds are maintained as 10 mM DMSO stocks.

On Day 0, MTAP-intact and MTAP-deleted cells are seeded in a 96-well plate, and incubated in a humidified, 5 or 10% CO₂ tissue culture incubator for 16-24 hours. On Day 1, the test compounds are dispensed to wells at defined concentrations using a Tecan D300e digital dispenser (n=3), and the volume of DMSO is normalized to highest class volume (0.2%). The compound-treated plates are incubated for 7 days in a humidified, 5 or 10% CO₂ tissue culture incubator.

On Day 7, the plates are removed from the tissue culture incubator and allowed to equilibrate to room temperature. Then either a ½ volume CellTiter-Glo Luminescent Cell Viability Assay reagent (Promega G7572) is added to each well, or the media is removed from every well and a 1:3 dilution of CellTiter-Glo 2.0 Cell Viability Assay reagent (Promega G9241) in 1×PBS is added. Ten minutes after addition, the luminescent signal is detected by an Envision plate reader. The data are plotted as % of the DMSO control wells for the MTAP-intact and the MTAP-deleted cell lines independently, and fitted to the 4-parameter logistic (4-PL) Hill equation with maximal effect constrained to 0. The fit was performed using GraphPad Prism or the default IC₅₀ fitting procedure in Dotmatics Studies 5.4 as part of a customized data analysis protocol.

The data obtained in this experiment is presented in Table 1, column 8.

### Example 121. Combination Viability Assay Protocol

A SW1573 MTAP-isogenic cell line pair can be generated by either reconstituting MTAP gene expression, or by introducing an empty control vector, in the MTAP-deleted SW1573 parental cell line. The cell lines can be maintained in DMEM+10% FBS in a humidified, 5% CO₂ tissue culture incubator. All test compounds can be maintained as 10 mM DMSO stocks.

On Day 0, MTAP-intact and MTAP-deleted cells can be seeded in a 384-well plate, and incubated in a humidified, 5% CO₂ tissue culture incubator for 16-24 hours. On Day 1, the test compounds can be dispensed to wells at defined concentrations (n=2), and the volume of DMSO can be normalized to highest class volume. The compound-treated plates can be incubated for 7 days in a humidified, 5% CO₂ tissue culture incubator.

On Day 7, the plates can be removed from the tissue culture incubator and allowed to equilibrate to room temperature. Relative viability can be assessed by addition of CellTiter-Glo reagent, and data can be plotted as % of DMSO control for each compound in each cell line, with a 4-parameter fit non-linear regression model (GraphPad Prism). Synergy can be determined according to the HSA model by the Combenefit software package (Version 2.021).

### Prmt5 Inhibitors and MAT2A Inhibitors Represents a Potential Clinical Combination in MTAP-Deleted Tumors

Marjon et al (Cell Reports 2016) and Kalev et al (Cancer Cell 2021) identify MAT2A as a therapeutic target in MTAP-deleted tumors. The combination of a MAT2A inhibitor with an inhibitor that selectively targets PRMT5 in MTAP-null cells can be assessed to determine whether this would present a rational therapeutic strategy. Combination of a MAT2A inhibitor (e.g., AG-270) with an exemplar MTAPnu-selective PRMT5 inhibitor in a 7-day viability assay in the MTAP-null SW1573 cancer cell line can demonstrate enhanced cellular viability defects.

### Prmt5 Inhibitors and MAPK or KRASG12C Inhibitors Represent a Potential Clinical Combination in MTAP-Deleted, KRAS-Mutated Tumors

MTAP-deletion can co-occur with mutations in the KRAS gene (e.g., KRASG12C). Therapies targeting KRAS or other members of the MAPK pathway (eg, MAPK3, MAPK1, MEK1 and MEK2) exist. The combination of these inhibitors with an inhibitor that selectively targets PRMT5 in MTAP-null cells can be assessed to determine whether this would present a therapeutic strategy.

Combination of a KRASG12C inhibitor (e.g., AMG-510), with an exemplar MTAP^{null}-selective PRMT5 inhibitor in a 7-day viability assay in the MTAP-null SW1573 cancer cell line can demonstrate enhanced cellular viability defects.

Combination of MAPK1/MAPK3 inhibitors (e.g., ulixertinib and SCH772984), with an exemplar MTAP^{null}-selective PRMT5 inhibitor in a 7-day viability assay in the MTAP-null SW1573 cancer cell line can demonstrate enhanced cellular viability defects.

Combination of MEK inhibitors (e.g., trametinib) with an exemplar MTAPnun—¹ selective PRMT5 inhibitor in a 7-day viability assay in the MTAP-null SW1573 cancer cell line can demonstrate enhanced cellular viability defects.

### Selected Embodiments

Embodiment 1. A compound of Formula (I) or a pharmaceutically acceptable salt thereof,
wherein:

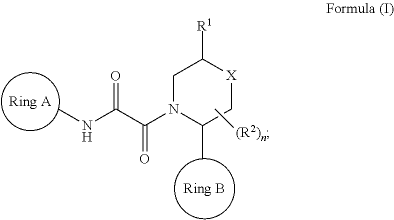

X is selected from the group consisting of —O— and —NR⁷—;
Ring A is selected from the group consisting of an optionally substituted fused bicyclic 8-10 membered heteroaryl ring system containing at least one nitrogen atom, wherein the 8-10 membered refers to the total number of atoms in the fused system and optionally substituted pyridin-3-yl;
Ring B is selected from the group consisting of C₆-C₁₀ aryl and 5-10 membered heteroaryl, each optionally substituted at any available position;
each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a1})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1}and —S(═O)₂N(R^{a11})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring);
each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(═O)₂N(R^{a7})₂wherein each alkyl, cycloalkyl, heterocyclyl, aryl, heteroaryl, arylalkyl and heteroarylalkyl is optionally substituted at any available position;
each R^{a1}, R^{a2} and W is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R, wherein each R is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R, —C(═O)OR, —NR^{b}C(═O)R^{cc}, —NR^{b}C(═O)OR^{cc}, —C(═O)N(R^{b})₂, —OC(═O)N(R)₂, —S(═O)R^{cc}, —S(═O)₂R^{b}, —SR^{b}, —S(═O)(═NR^{b})R, —NR^{b}S(═O)₂R and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -Bu, -sec-Bu, -iso-Bu).and C₃-C₉ cycloalkyl (e.g., cyclopropyl, cyclobutyl, cyclopentyl, cyclohexyl); and
n is 0, 1, 2 or 3;

Embodiment 2. The compound of embodiment 1, or a pharmaceutically acceptable salt thereof, wherein R¹ is not absent or H and Ring B and R¹ are in a trans relative configuration.

Embodiment 3. The compound of embodiment 1, or a pharmaceutically acceptable salt thereof, wherein R¹ is not absent or H and Ring B and R¹ are in a cis relative configuration.

Embodiment 4. The compound of embodiment 1, or a pharmaceutically acceptable salt thereof, wherein the moiety represented as

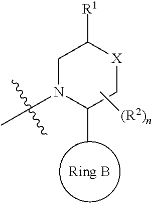

is selected from the group consisting of:

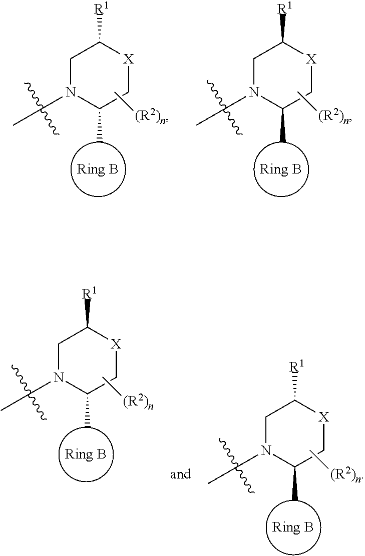

Embodiment 5. The compound of any one of embodiments 1, 2 and 4, or a pharmaceutically acceptable salt thereof, wherein the moiety represented as

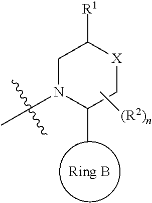

is selected from the group consisting of:

Embodiment 6. The compound of any one of embodiments 1, 3 and 4, or a pharmaceutically acceptable salt thereof, wherein the moiety represented as

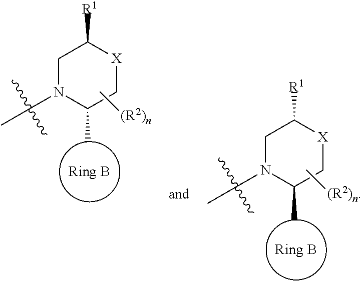

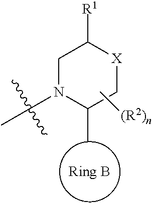

is selected from the group consisting of:

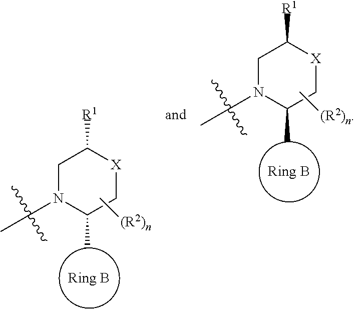

Embodiment 7. The compound of embodiment 5, or a pharmaceutically acceptable salt thereof, wherein the compound is of Formula (Ia) or Formula (Ib):

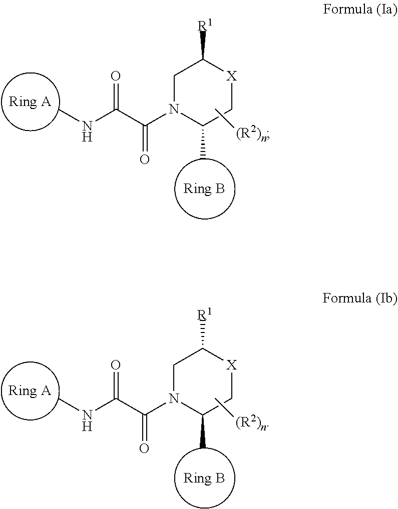

Embodiment 8. The compound of embodiment 5, or a pharmaceutically acceptable salt thereof, wherein the compound is of Formula (Ia).

Embodiment 9. The compound of embodiment 5, or a pharmaceutically acceptable salt thereof, wherein the compound is of Formula (Ib).

Embodiment 10. The compound of embodiment 6, or a pharmaceutically acceptable salt thereof, wherein the compound is of Formula (Ic) or Formula (Id):

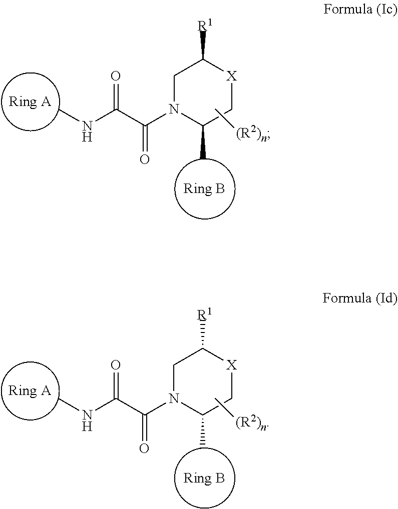

Embodiment 11. The compound of embodiment 6, or a pharmaceutically acceptable salt thereof, wherein the compound is of Formula (Ic).

Embodiment 12. The compound of embodiment 6, or a pharmaceutically acceptable salt thereof, wherein the compound is of Formula (Id).

Embodiment 13. The compound of any one of embodiments 1-12 or a pharmaceutically acceptable salt thereof, wherein X is —O—.

Embodiment 14. The compound of embodiment 1 or a pharmaceutically acceptable salt thereof, wherein the compound is of formula (II):

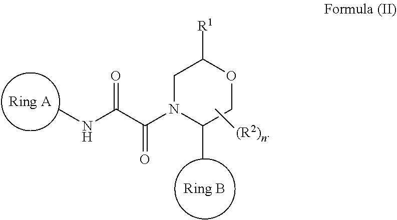

Embodiment 15. The compound of embodiment 14, or a pharmaceutically acceptable salt thereof, wherein the compound is of Formula (IIa), Formula (IIb), Formula (IIc) or Formula (IId):

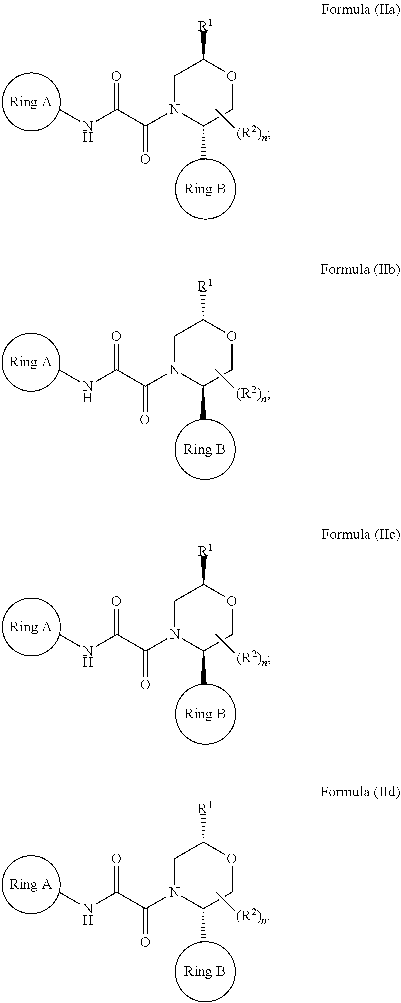

Embodiment 16. The compound of embodiment 14, or a pharmaceutically acceptable salt thereof, wherein the compound is of Formula (IIa) or Formula (IIb).

Embodiment 17. The compound of embodiment 14, or a pharmaceutically acceptable salt thereof, wherein the compound is of Formula (IIa).

Embodiment 18. The compound of embodiment 14, or a pharmaceutically acceptable salt thereof, wherein the compound is of Formula (IIb).

Embodiment 19. The compound of any one of embodiments 1-12 or a pharmaceutically acceptable salt thereof, wherein X is —NR⁷-.

Embodiment 20. The compound of embodiment 1 or a pharmaceutically acceptable salt thereof, wherein the compound is of formula (III):

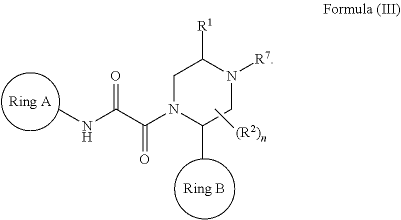

Embodiment 21. The compound of embodiment 20, or a pharmaceutically acceptable salt thereof, wherein the compound is of Formula (IIIa), Formula (IIIb), Formula (IIIc) or Formula (IIId):

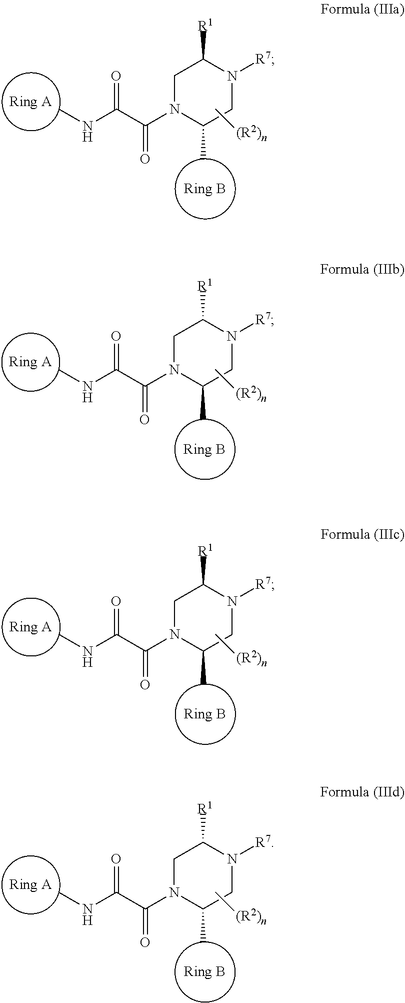

Embodiment 22. The compound of embodiment 20, or a pharmaceutically acceptable salt thereof, wherein the compound is of Formula (I_IIa) or Formula (IIIb).

Embodiment 23. The compound of embodiment 20, or a pharmaceutically acceptable salt thereof, wherein the compound is of Formula (IIIa).

Embodiment 24. The compound of embodiment 20, or a pharmaceutically acceptable salt thereof, wherein the compound is of Formula (IIIb).

Embodiment 25. The compound of any one of embodiments 1 to 24, or a pharmaceutically acceptable salt thereof, wherein Ring A is selected from the group consisting of an optionally substituted fused bicyclic 8-10 membered heteroaryl ring system containing at least one nitrogen atom, wherein the 8-10 membered refers to the total number of atoms in the fused system and pyridin-3-yl, each substituted at any available positions with 0, 1, 2, 3 or 4 instances of R⁴, wherein:
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, NR^{a4}S(═O)₂R^{a4} and —S(═O)₂N(R^{a4})₂; and
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR, —NR^{b}C(═O)R^{b}, —NR^{b}C(═O)OR^{cc}, —C(═O)N(R^{b})₂, —OC(═O)N(R^{b})₂, —S(═O)R, —S(═O)₂R, —SR, —S(═O)(═NR^{b})R^{b}, —NR^{b}S(═O)₂R^{b} and —S(=O)₂N(R^{b})₂, wherein each R^{b} is independently selected from the group consisting of H, —C₁-C₆ alkyl (e.g., -Me, -Et, —Pr, —ⁱPr, -^{tt}Bu, -Bu, -sec-Bu, -iso-Bu)), and C₃-C₉ cycloalkyl (e.g., cyclopropyl, cyclobutyl, cyclopentyl, cyclohexyl).

Embodiment 26. The compound of any one of embodiments 1 to 25, or a pharmaceutically acceptable salt thereof, wherein Ring A is selected from the group consisting of:

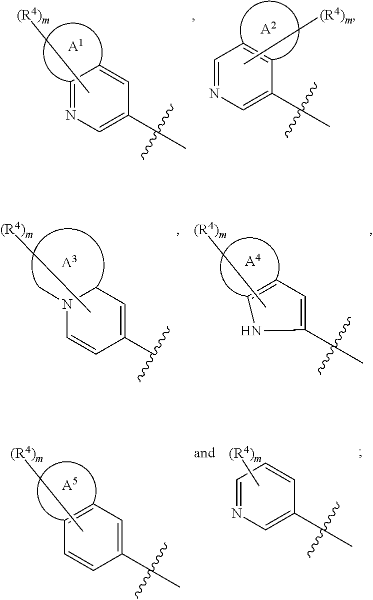

wherein
each of rings A¹, A² and A⁴ is independently 4-6 membered carbocyclyl, 4-6 membered heterocyclyl, 5-6 membered heteroaryl or phenyl;
each ring A³ is independently a 4-6 membered heterocyclyl or 5-6 membered heteroaryl, wherein the heterocyclyl and heteroaryl contain at least one nitrogen atom
each ring A⁵ is independently a 5-6 membered heteroaryl, wherein the heteroaryl contains at least one nitrogen atom;
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, NR^{a4}S(═O)₂R^{a4} and —S(═O)₂N(R^{a4})₂;
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR^{cc}, —NR^{b}C(═O)R, —NR^{b}C(═O)OR^{cc}, —C(═O)N(R^{b})₂, —OC(═O)N(R^{b})₂, —S(═O)R, —S(═O)₂R^{cc}, —SR^{b}, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu)), and C₃-C₉ cycloalkyl (e.g., cyclopropyl, cyclobutyl, cyclopentyl, cyclohexyl); and
m is 0, 1,2, 3 or 4.

Embodiment 27. The compound of embodiment 26, or a pharmaceutically acceptable salt thereof, wherein Ring A is selected from the group consisting of:

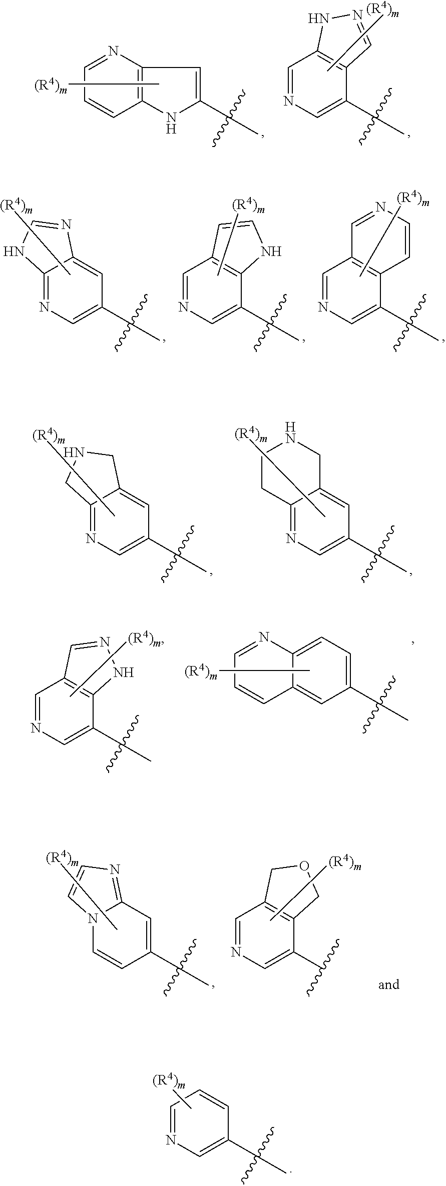

Embodiment 28. The compound of embodiment 26, or a pharmaceutically acceptable salt thereof, wherein Ring A is selected from the group consisting of:

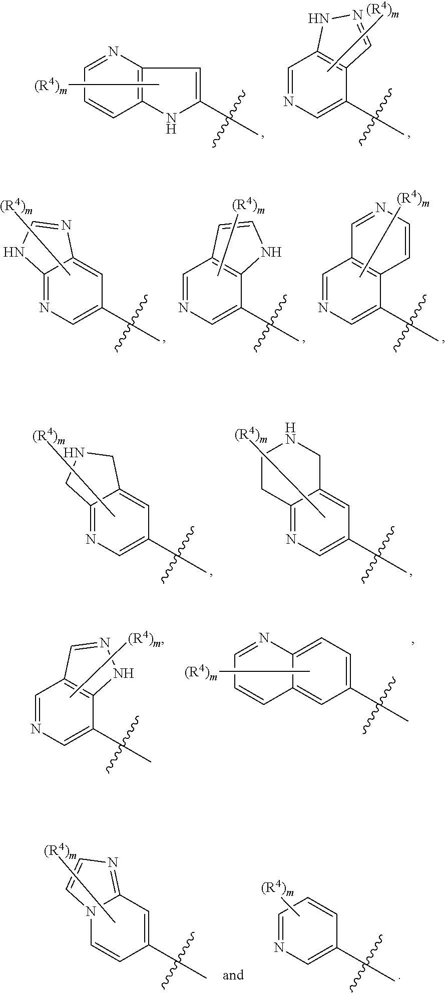

Embodiment 29. The compound of embodiment 26, or a pharmaceutically acceptable salt thereof, wherein Ring A is selected from the group consisting of:

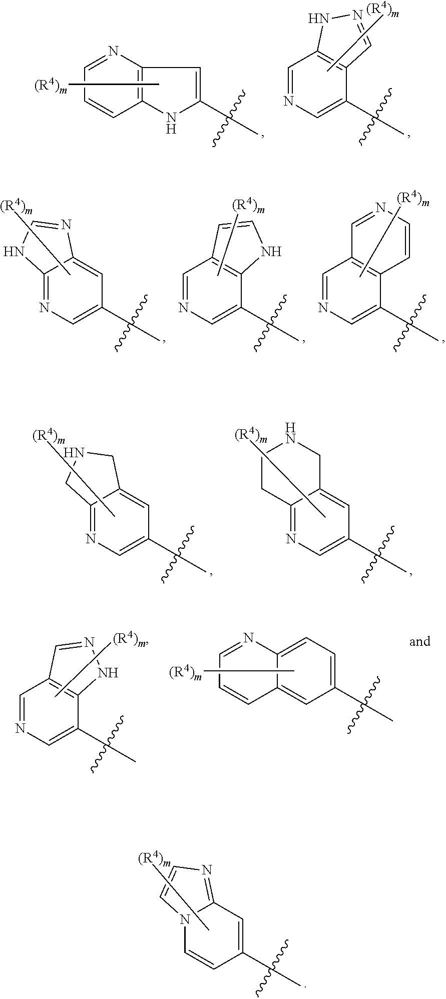

Embodiment 30. The compound of embodiment 26, or a pharmaceutically acceptable salt thereof, wherein Ring A is selected from the group consisting of:

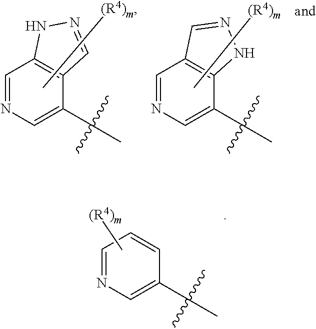

Embodiment 31. The compound of embodiment 26, or a pharmaceutically acceptable salt thereof, wherein Ring A is:

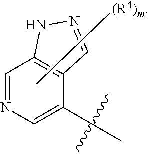

Embodiment 32. The compound of embodiment 26, or a pharmaceutically acceptable salt thereof, wherein Ring A is:

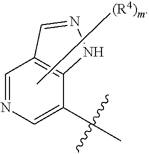

Embodiment 33. The compound of embodiment 26, or a pharmaceutically acceptable salt thereof, wherein Ring A is:

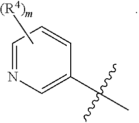

Embodiment 34. The compound of any one of embodiments 26 to 33, or a pharmaceutically acceptable salt thereof, wherein m is 0, 1 or 2.

Embodiment 35. The compound of any one of embodiments 26 to 33, or a pharmaceutically acceptable salt thereof, wherein m is 0 or 1.

Embodiment 36. The compound of any one of embodiments 26 to 33, or a pharmaceutically acceptable salt thereof, wherein m is 0.

Embodiment 37. The compound of any one of embodiments 26 to 33, or a pharmaceutically acceptable salt thereof, wherein m is 1.

Embodiment 38. The compound of any one of embodiments 26 to 33, or a pharmaceutically acceptable salt thereof, wherein m is 2.

Embodiment 39. The compound of any one of embodiments 26 to 33, or a pharmaceutically acceptable salt thereof, wherein m is 3.

Embodiment 40. The compound of embodiment 27, or a pharmaceutically acceptable salt thereof, wherein Ring A is selected from the group consisting of:

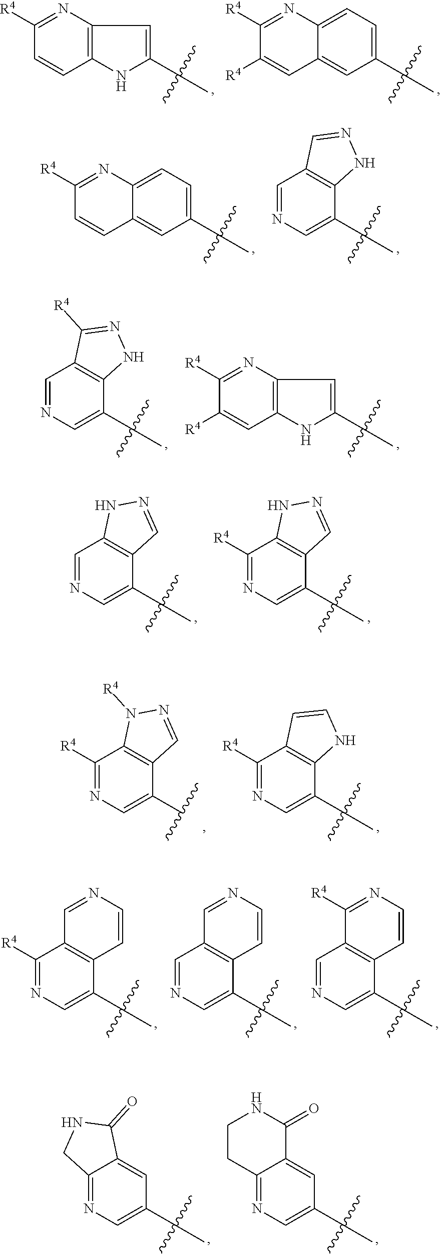

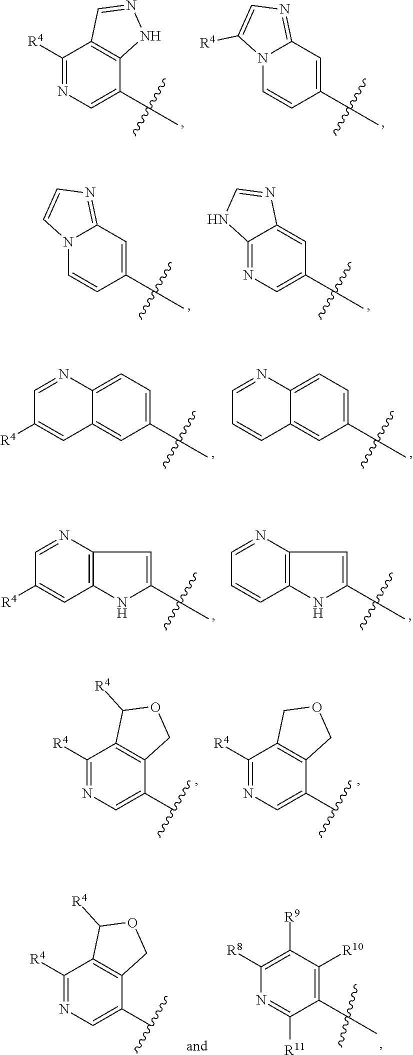

wherein
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁸ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a8}, —N(R^{a8})₂, —C(═O)R^{a8}, —C(═O)OR^{a8}, —NR^{a8}C(═O)R^{a8}, —NR^{a8}C(═O)OR^{a8}, —C(═O)N(R^{a8})₂, —OC(═O)N(R^{a8})₂, —S(═O)R^{a8}, —S(═O)₂R^{a8}, —SR^{a8}, —S(═O)(═NR^{a8})R^{a8}, —NR^{a8}S(═O)₂R^{as} and —S(═O)₂N(R^{a8})₂;
each R⁹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a9}, —N(R^{a9})₂, —C(═O)R^{a9}, —C(═O)OR^{a9}, —NR^{a9}C(═O)R^{a9}, —NR^{a9}C(═O)OR^{a9}, —C(═O)N(R^{a9})₂, —OC(═O)N(R^{a9})₂, —S(═O)R^{a9}, —S(═O)₂R^{a9}, —SR^{a9}, —S(═O)(═NR^{a9})R^{a9}, —NR^{a9}S(═O)₂R^{a9} and —S(═O)₂N(R^{a9})₂;
each R¹⁰ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a10}, —N(R^{a10})₂—C(═O)R^{a10}, —C(═O)OR^{a10}, —NR^{a1}OC(═O)R^{a10}, —NR^{a10}C(═O)OR^{a10}, —C(═O)N(R^{a10})₂—OC(═O)N(R^{a10})₂, —S(═O)R^{a10}, —S(═O)₂R^{a10}, —SR^{a10}, —S(═O)(═NR^{a10})R^{a10}, —NR^{a10}S(═O)₂R^{a10} and —S(═O)₂N(R^{a10})₂;
each R¹¹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(R^{a1})₂, —C(═O)R^{a11}, —C(═O)OR^{a11}, —NR^{a11}C(═O)Ran, —NR^{a11}C(═O)OR^{a1}, —C(═O)N(Ra)₂, —OC(═O)N(Ran)₂, —S(═O)Ran, —S(═O)₂R^{a11}—SR^{a11}—S(═O)(═NR^{a11})R^{a11}—NR^{a11}S(═O)₂Ran and —S(═O)₂N(Ra)₂;
each R^{a4}, R^{a8}, R^{a9}, R^{a10} and R^{a11} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R, wherein each R is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{cc}, —C(═O)OR, —NR^{b}C(═O)R^{cc}, —NR^{b}C(═O)OR, —C(═O)N(R^{b})₂, —OC(═O)N(R^{b})₂, —S(═O)R^{cc}, —S(═O)₂R^{b}, —SR^{b}, —S(═O)(═NR^{b})R, —NR^{b}S(═O)₂R and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl).

Embodiment 41. The compound of embodiment 28, or a pharmaceutically acceptable salt thereof, wherein Ring A is selected from the group consisting of:

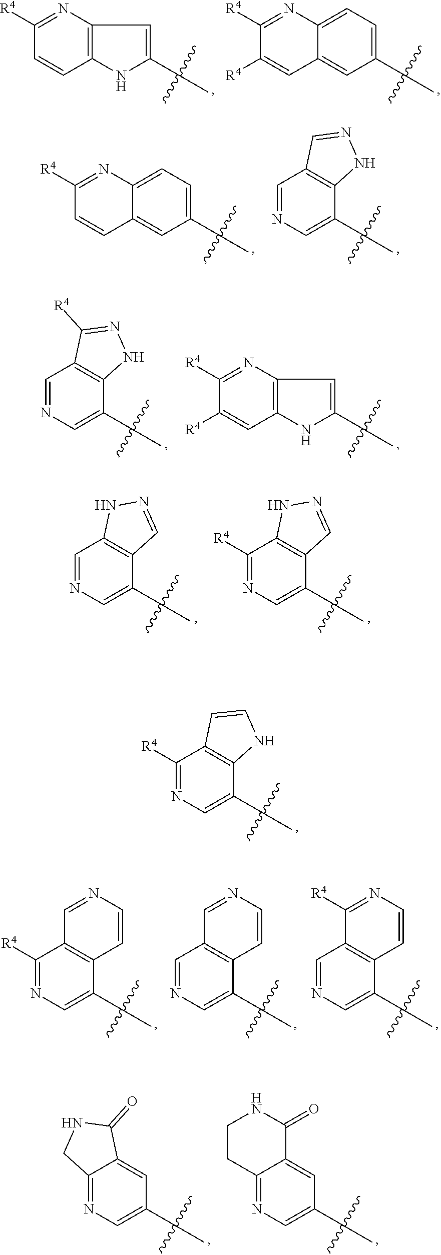

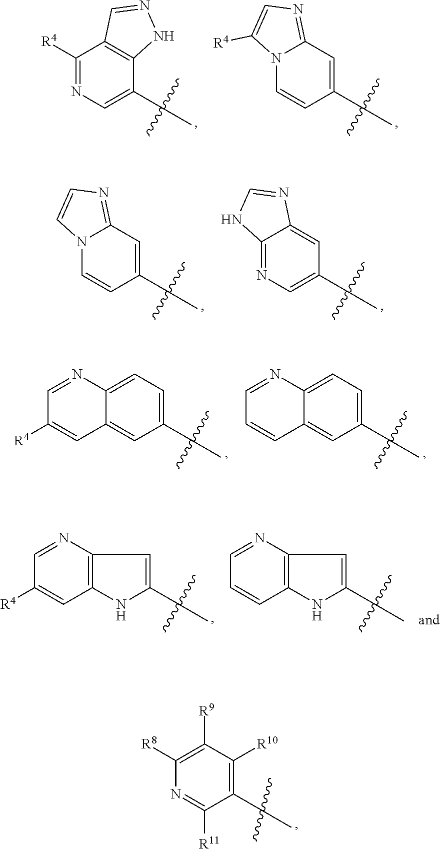

wherein
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁸ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a8}, —N(R^{a8})₂, —C(═O)R^{a8}, —C(═O)OR^{a8}, —NR^{a8}C(═O)R^{a8}, —NR^{a8}C(═O)OR^{a8}, —C(═O)N(R^{a8})₂, —OC(═O)N(R^{a8})₂, —S(═O)R^{a8}, —S(═O)₂R^{a8}, —SR^{a8}, —S(═O)(═NR^{a8})R^{a8}, —NR^{a8}S(═O)₂R^{as} and —S(═O)₂N(R^{a8})₂;
each R⁹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a9}, —N(R^{a9})₂, —C(═O)R^{a9}, —C(═O)OR^{a9}, —NR^{a9}C(═O)R^{a9}, —NR^{a9}C(═O)OR^{a9}, —C(═O)N(R^{a9})₂, —OC(═O)N(R^{a9})₂, —S(═O)R^{a9}, —S(═O)₂R^{a9}, —SR^{a9}, —S(═O)(═NR^{a9})R^{a9}, —NR^{a9}S(═O)₂R^{a9} and —S(═O)₂N(R^{a9})₂;

each R¹⁰ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a10}, —N(R^{a10})₂—C(═O)R^{a10}, —C(═O)OR^{a10}, —NR^{a10}C(═O)R^{a10}, —NR^{a10}C(═O)OR^{a10}, —C(═O)N(R^{a10})₂, —OC(═O)N(R^{a10})₂, —S(═O)R^{a10}, —S(═O)₂R^{a10}, —SR^{a10}, —S(═O)(═NR^{a10})R^{a10}, —NR^{a10}S(═O)₂R^{a10} and —S(═O)₂N(R^{a10})₂;
each R¹¹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}, —N(Ra)₂, —C(═O)R^{a11}, —C(═O)ORa¹¹, —NR^{a11}C(═O)R^{a1}, —NR^{a11}1C(═O)OR^{a11}, —C(═O)N(Ra)₂, —OC(═O)N(Ra)₂, —S(═O)R^{a11}, —S(═O)₂R^{a11}, —SR^{a11}, —S(═O)(═NR^{a11})R^{a11}, —NR^{a11}S(═O)₂Ran and —S(═O)₂N(Ra)₂;
each R⁴, R^{a8}, R^{a9}, R^{a10} and R^{a11} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R, —C(═O)OR, —NR^{b}C(═O)R, —NR^{b}C(═O)OR, —C(═O)N(R^{b})₂, —OC(═O)N(R^{b})₂, —S(═O)R^{cc}, —S(═O)₂R^{b}, —SR^{b}, —S(=O)(═NR^{b})R, —NR^{b}S(═O)₂R and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl).

Embodiment 42. The compound of embodiment 41, or a pharmaceutically acceptable salt thereof, wherein Ring A is selected from the group consisting of:

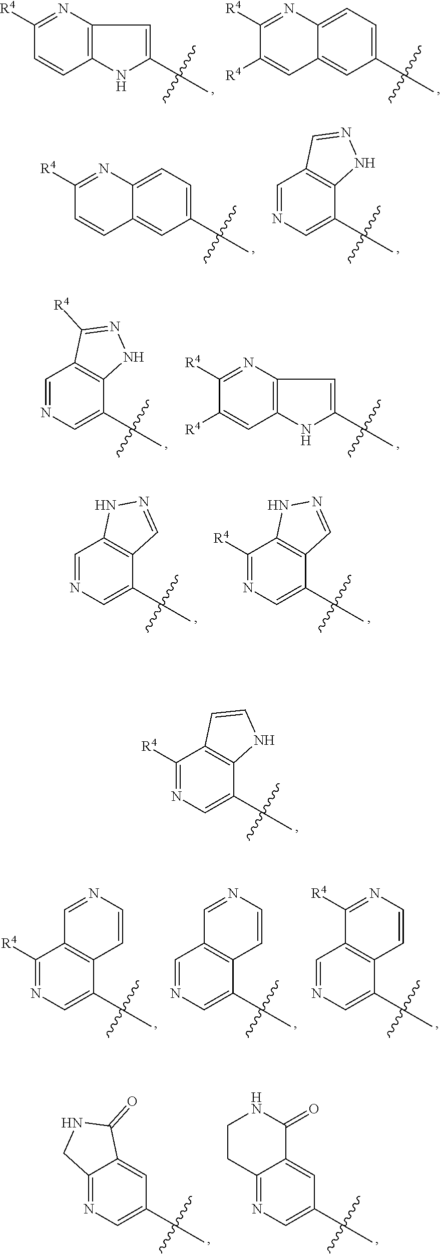

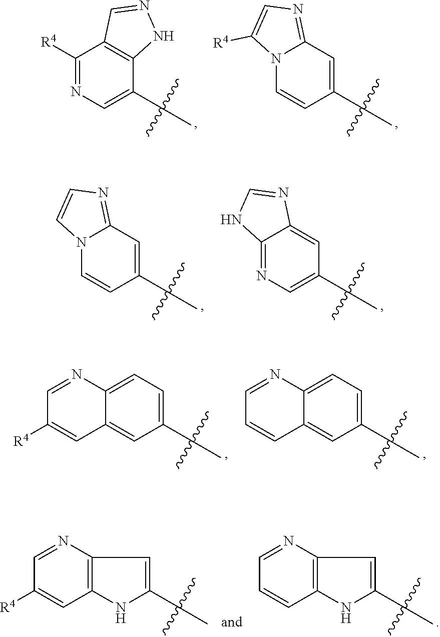

Embodiment 43. The compound of embodiment 40, or a pharmaceutically acceptable salt thereof, wherein Ring A is selected from the group consisting of:

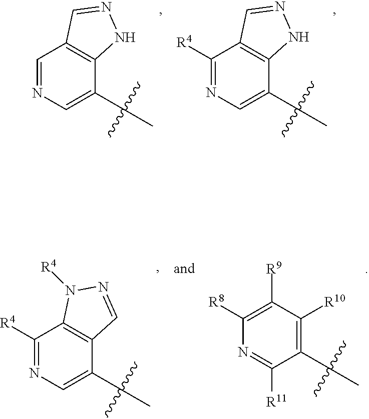

Embodiment 44. The compound of embodiment 40, or a pharmaceutically acceptable salt thereof, wherein Ring A is selected from the group consisting of:

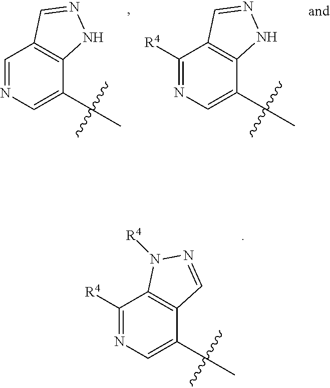

Embodiment 45. The compound of embodiment 40 or 41, or a pharmaceutically acceptable salt thereof, wherein Ring A is selected from the group consisting of:

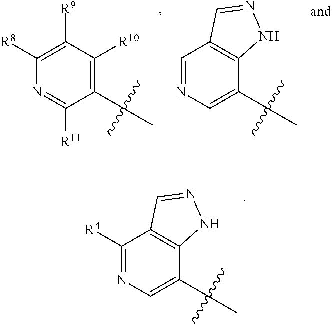

Embodiment 46. The compound of embodiment 40 or 41, or a pharmaceutically acceptable salt thereof, wherein Ring A is selected from the group consisting of:

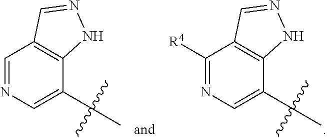

Embodiment 47. The compound of embodiment 40 or 41, or a pharmaceutically acceptable salt thereof, wherein Ring A is

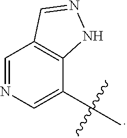 [SMILES: *c1cncc2cn[nH]c12]

Embodiment 48. The compound of embodiment 40 or 41, or a pharmaceutically acceptable salt thereof, wherein Ring A is

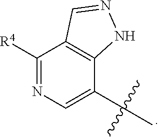

Embodiment 49. The compound of embodiment 40, or a pharmaceutically acceptable salt thereof, wherein Ring A is

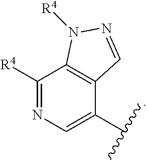

Embodiment 50. The compound of embodiment 40, or a pharmaceutically acceptable salt thereof, wherein Ring A is

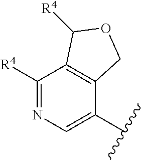

Embodiment 51. The compound of embodiment 40 or 41, or a pharmaceutically acceptable salt thereof, wherein Ring A is

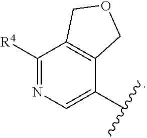

Embodiment 52. The compound of any one of embodiments 25 to 51, or a pharmaceutically acceptable salt thereof, wherein each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, —OR^{a4}, —N(R⁴)₂, —C(═O)R^{a4}, —C(═O)OR^{a4}, —NR^{a4}C(═O)R^{a4}, NR^{a4}C(═O)OR^{a4}, —C(═O)N(R^{a4})₂, —C(═O)N(OR^{a4})(R^{a4}) and —OC(═O)N(R^{a4})₂.

Embodiment 53. The compound of any one of embodiments 25 to 51, or a pharmaceutically acceptable salt thereof, wherein each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, —OR^{a4}, —N(R⁴)₂, —C(═O)R^{a4}, —C(═O)OR^{a4}, —NR^{a4}C(═O)R^{a4}, NR^{a4}C(═O)OR^{a4}, —C(═O)N(R^{a4})₂, —C(═O)N(OR^{a4})(R^{a4}) and —OC(═O)N(R^{a4})₂.

Embodiment 54. The compound of any one of embodiments 25 to 51, or a pharmaceutically acceptable salt thereof, wherein each R⁴ is independently selected from the group consisting of -D, ═O, —C₁-C₆ alkyl, —C₁-C₆ haloalkyl, —C₃-C₉ cycloalkyl, —OR⁴ and —N(R^{a4})₂.

Embodiment 55. The compound of any one of embodiments 25 to 51, or a pharmaceutically acceptable salt thereof, wherein each R⁴ is independently selected from the group consisting of -D, ═O, —C₁-C₆ alkyl, —C₁-C₆ haloalkyl, —C₃-C₉ cycloalkyl, —OR⁴ and -20 N(R^{a4})₂.

Embodiment 56. The compound of any one of embodiments 25 to 51, or a pharmaceutically acceptable salt thereof, wherein each R⁴ is independently selected from the group consisting of ═O, —C₁-C₆ alkyl, —C₁-C₆ haloalkyl, —C₃-C₉ cycloalkyl, —OR^{a4} and —N(R^{a4})₂.

Embodiment 57. The compound of any one of embodiments 25 to 51, or a pharmaceutically acceptable salt thereof, wherein each R⁴ is independently selected from the group consisting of -D, ═O, —C₁-C₆ alkyl and —N(R^{a4})₂.

Embodiment 58. The compound of any one of embodiments 25 to 51, or a pharmaceutically acceptable salt thereof, wherein each R⁴ is independently selected from the group consisting of ═O, —C₁-C₆ alkyl and —N(R^{a4})₂.

Embodiment 59. The compound of any one of embodiments 25 to 58, or a pharmaceutically acceptable salt thereof, wherein each R^{a4} is independently selected from the group consisting of H and —C₁-C₆ alkyl.

Embodiment 60. The compound of any one of embodiments 25 to 58, or a pharmaceutically acceptable salt thereof, wherein each R^{a4} is H.

Embodiment 61. The compound of any one of embodiments 25 to 51, or a pharmaceutically acceptable salt thereof, wherein each R⁴ is independently selected from the group consisting of ═O, -Me, -Et, —ⁱPr, -^{t}Bu, —NH₂, —NHCH₃ and —NH(CH₃)₂.

Embodiment 62. The compound of any one of embodiments 25 to 51, or a pharmaceutically acceptable salt thereof, wherein each R⁴ is independently —NHCH₃, —NH₂ or -Me.

Embodiment 63. The compound of any one of embodiments 25 to 51, or a pharmaceutically acceptable salt thereof, wherein each R⁴ is independently —NH₂ or -Me.

Embodiment 64. The compound of any one of embodiments 25 to 51, or a pharmaceutically acceptable salt thereof, wherein each R⁴ is —NH₂.

Embodiment 65. The compound of any one of embodiments 25 to 51, or a pharmaceutically acceptable salt thereof, wherein each R⁴ is -Me.

Embodiment 66. The compound of any one of embodiments 40 or 41, or a pharmaceutically acceptable salt thereof, wherein Ring A is

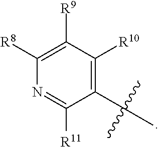

Embodiment 67. The compound of any one of embodiments 40, 41, 43, 45 and 66, or a pharmaceutically acceptable salt thereof, wherein R¹⁰ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a10}, —N(R^{a10})₂—C(═O)R^{a10}, —C(═O)OR^{a10}, —NR^{a10}C(═O)R^{a10}, —NR^{a10}C(═O)OR^{a10}, —C(═O)N(R^{a10})₂ and —OC(═O)N(R^{a10})₂.

Embodiment 68. The compound of any one of embodiments 40, 41, 43, 45 and 66, or a pharmaceutically acceptable salt thereof, wherein R¹⁰ is selected from the group consisting of H, halo, —CN, —C₁-C₆ alkyl, —C₁-C₆ haloalkyl and —N(R^{a10}).

Embodiment 69. The compound of any one of embodiments 40, 41, 43, 45 and 66-68, or a pharmaceutically acceptable salt thereof, wherein R^{a10} is selected from the group consisting of H and —C₁-C₆ alkyl (e.g., -Me, -Et, —Pr, -iPr, -nBu, -tBu, -sec-Bu, -iso-Bu).

Embodiment 70. The compound of any one of embodiments 40, 41, 43, 45 and 66, or a pharmaceutically acceptable salt thereof, wherein R¹⁰ is selected from the group consisting of —C₁-C₆ alkyl (e.g., -Me, -Et, —Pr, -iPr, -nBu, -tBu, -sec-Bu, -iso-Bu), —OH, -0-(C₁-C₆ alkyl) (e.g., —OCH₃), —NH₂, —NH—(C₁-C₆ alkyl) (e.g., —NHCH₃) and —N—(C₁-C₆ alkyl)₂ (e.g, —N(CH₃)₂).

Embodiment 71. The compound of any one of embodiments 40, 41, 43, 45 and 66, or a pharmaceutically acceptable salt thereof, wherein R¹⁰ is selected from the group consisting of H and -Me.

Embodiment 72. The compound of any one of embodiments 40, 41, 43, 45 and 66, or a pharmaceutically acceptable salt thereof, wherein R¹⁰ is —H.

Embodiment 73. The compound of any one of embodiments 40, 41, 43, 45 and 66-72, or a pharmaceutically acceptable salt thereof, wherein R¹¹ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a11}, —N(Ra)₂, —C(═O)R^{a11}, —C(═O)OR^{a11}, —NR^{a11}C(═O)R^{a1}, —NR^{a11}C(═O)OR^{a1}, —C(═O)N(Ra)₂ and —OC(═O)N(R^{a11})₂.

Embodiment 74. The compound of any one of embodiments 40, 41, 43, 45 and 66-72, or a pharmaceutically acceptable salt thereof, wherein R¹¹ is selected from the group consisting of H, halo, —CN, —C₁-C₆ alkyl, —C₁-C₆ haloalkyl and —N(Ra)₂.

Embodiment 75. The compound of embodiment 40, 41, 43, 45 and 66-74, or a pharmaceutically acceptable salt thereof, wherein each R^{a11} is independently selected from the group consisting of H and —C₁-C₆ alkyl (e.g., -Me, -Et, —Pr, -iPr, -nBu, -tBu, -sec-Bu, -iso-Bu).

Embodiment 76. The compound of any one of embodiments 40, 41, 43, 45 and 66-72, or a pharmaceutically acceptable salt thereof, wherein R¹¹ is selected from the group consisting of —C₁-C₆ alkyl (e.g., -Me, -Et, —Pr, -iPr, -nBu, -tBu, -sec-Bu, -iso-Bu), —OH, -0-(C₁-C₆ alkyl) (e.g., —OCH₃), —NH₂, —NH—(C₁-C₆ alkyl) (e.g., —NHCH₃) and —N—(C₁-C₆ alkyl)₂ (e.g, —N(CH₃)₂).

Embodiment 77. The compound of any one of embodiments 40, 41, 43, 45 and 66-72, or a pharmaceutically acceptable salt thereof, wherein R¹¹ is H.

Embodiment 78. The compound of any one of embodiments 40, 41, 43, 45 and 66, or a pharmaceutically acceptable salt thereof, wherein ring A is

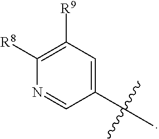

Embodiment 79. The compound of any one of embodiments 40, 41, 43, 45 and 66-78, or a pharmaceutically acceptable salt thereof, wherein R⁸ is selected from the group consisting of H, halo, —CN, —C₁-C₆ alkyl, —C₁-C₆ heteroalkyl, —C₁-C₆ haloalkyl, —C₃-C₉ cycloalkyl, 3-10 membered heterocyclyl, —OR^{a8}, —N(R^{a8})₂, —C(═O)R^{a8}, —C(═O)OR^{a8}, —NR^{a8}C(═O)R^{a8}, —NR^{a8}C(═O)OR^{a8}, —C(═O)N(R^{a8})₂ and —OC(═O)N(R^{a8})₂.

Embodiment 80. The compound of any one of embodiments 40, 41, 43, 45 and 66-78, or a pharmaceutically acceptable salt thereof, wherein R⁸ is selected from the group consisting of H, halo, —CN, —C₁-C₆ alkyl, —C₁-C₆ haloalkyl, —OR^{a8} and —N(R^{a8})₂.

Embodiment 81. The compound of any one of embodiments 40, 41, 43, 45 and 66-78, or a pharmaceutically acceptable salt thereof, wherein R⁸ is selected from the group consisting of H, —C₁-C₆ alkyl, —C₁-C₆ haloalkyl, —OR^{a8} and —N(R^{a8})₂.

Embodiment 82. The compound of any one of embodiments 40, 41, 43, 45 and 66-78, or a pharmaceutically acceptable salt thereof, wherein R⁸ is selected from the group consisting of —OR^{a8} and —N(R^{a8})₂.

Embodiment 83. The compound of any one of embodiments 40, 41, 43, 45 and 66-82, or a pharmaceutically acceptable salt thereof, wherein each R^{a8} is independently selected from the group consisting of H, —C₁-C₆ alkyl (e.g., -Me, -Et, —Pr, —ⁱPr, -nBu, -^{t}Bu, -sec-Bu, -iso-Bu) and —C₁-C₆ haloalkyl (e.g., —CHF₂, —CF₃).

Embodiment 84. The compound of any one of embodiments 40, 41, 43, 45 and 66-78, or a pharmaceutically acceptable salt thereof, wherein R⁸ is selected from the group consisting of H, —C₁-C₆ alkyl (e.g., -Me, -Et, —Pr, -iPr, -nBu, -tBu, -sec-Bu, -iso-Bu), —C₁-C₆ alkyl (e.g., —CF₃, —CHF₂), —OH, —O—(C₁-C₆ alkyl) (e.g., —OCH₃, -OEt), —O—(C₁-C₆ haloalkyl) (e.g., —OCF₃, —OCHF₂), —NH₂, —NH—(C₁-C₆ alkyl) (e.g., —NHCH₃) and —N—(C₁-C₆ alkyl)₂ (e.g, —N(CH₃)₂).

Embodiment 85. The compound of any one of embodiments 40, 41, 43, 45 and 66-78, or a pharmaceutically acceptable salt thereof, wherein R⁸ is selected from the group consisting of H, -Me, —CHF₂, —OCH₃ and —NH₂.

Embodiment 86. The compound of any one of embodiments 40, 41, 43, 45 and 66-78, or a pharmaceutically acceptable salt thereof, wherein R⁸ is selected from the group consisting of NH₂ and —OCH₃.

Embodiment 87. The compound of any one of embodiments 40, 41, 43, 45 and 66-78, or a pharmaceutically acceptable salt thereof, wherein R⁸ is —OCH₃.

Embodiment 88. The compound of any one of embodiments 40, 41, 43, 45 and 66-78, or a pharmaceutically acceptable salt thereof, wherein R⁸ is —NH₂.

Embodiment 89. The compound of any one of embodiments 40, 41, 43, 45 and 66-88, or a pharmaceutically acceptable salt thereof, wherein R⁹ is selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-10 membered heterocyclyl, —OR^{a9}, —N(R^{a9})₂, —C(═O)R^{a9}, C(═O)OR^{a9}, —NR^{a9}C(═O)R^{a9}, —NR^{a9}C(═O)OR^{a9}, —C(═O)N(R^{a9})₂, —OC(═O)N(R^{a9})₂.

Embodiment 90. The compound of any one of embodiments 40, 41, 43, 45 and 66-88, or a pharmaceutically acceptable salt thereof, wherein R⁹ is selected from the group consisting of halo, —CN, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a9}, —N(R^{a9})₂, —C(═O)R^{a9} and —C(═O)N(R^{a9})₂.

Embodiment 91. The compound of any one of embodiments 40, 41, 43, 45 and 66-88, or a pharmaceutically acceptable salt thereof, wherein R⁹ is selected from the group consisting of halo, —C₁-C₆ alkyl, —C₁-C₆ haloalkyl, 3-10 membered heterocyclyl (e.g., oxetanyl), —C₃-C₉ cycloalkyl (e.g., cyclopropyl), —OR^{a9}, —C(═O)R^{a9} and —C(═O)N(R^{a9})₂.

Embodiment 92. The compound of any one of embodiments 40, 41, 43, 45 and 66-88, or a pharmaceutically acceptable salt thereof, wherein R⁹ is selected from the group consisting of —C₁-C₆ alkyl, 3-10 membered heterocyclyl (e.g., oxetanyl), —C₃-C₉ cycloalkyl (e.g., cyclopropyl) and —C(═O)N(R^{a9})₂.

Embodiment 93. The compound of any one of embodiments 40, 41, 43, 45 and 66-92, or a pharmaceutically acceptable salt thereof, wherein each R^{a9} is independently selected from the group consisting of H and —C₁-C₆ alkyl (e.g., -Me, -Et, —Pr, -iPr, -nBu, -tBu, -sec-Bu, -iso-Bu).

Embodiment 94. The compound of any one of embodiments 40, 41, 43, 45 and 66-88, or a pharmaceutically acceptable salt thereof, wherein R⁹ is selected from the group consisting of —C₁-C₆ alkyl (e.g., -Me, -Et, —Pr, -iPr, -nBu, -tBu, -sec-Bu, -iso-Bu), 3-10 membered heterocyclyl (e.g., oxetan-3-yl), —C₃-C₉ cycloalkyl (e.g., cyclopropyl) and —C(═O)NH₂.

Embodiment 95. The compound of any one of embodiments 40, 41, 43, 45 and 66-88, or a pharmaceutically acceptable salt thereof, wherein R⁹ is selected from the group consisting of -Me, -Et, oxetan-3-yl, cyclopropyl and —C(═O)NH₂.

Embodiment 96. The compound of any one of embodiments 40, 41, 43, 45 and 66-88, or a pharmaceutically acceptable salt thereof, wherein R⁹ is —C(═O)NH₂.

Embodiment 97. The compound of any one of embodiments 40, 41, 43, 45 and 66-88, or a pharmaceutically acceptable salt thereof, wherein R⁹ is selected from the group consisting of -Me, -Et, oxetan-3-yl and cyclopropyl.

Embodiment 98. The compound of any one of embodiments 40, 41, 43, 45 and 66-78, or a pharmaceutically acceptable salt thereof, wherein R⁸ is —OCH₃ and R⁹ is —C(═O)NH₂.

Embodiment 99. The compound of any one of embodiments 40, 41, 43, 45 and 66-78, or a pharmaceutically acceptable salt thereof, wherein R⁸ is —NH₂ and R⁹ is selected from the group consisting of -Me, -Et, oxetan-3-yl, cyclopropyl.

Embodiment 100. The compound of embodiment 40, or a pharmaceutically acceptable salt thereof, wherein Ring A is selected from the group consisting of:

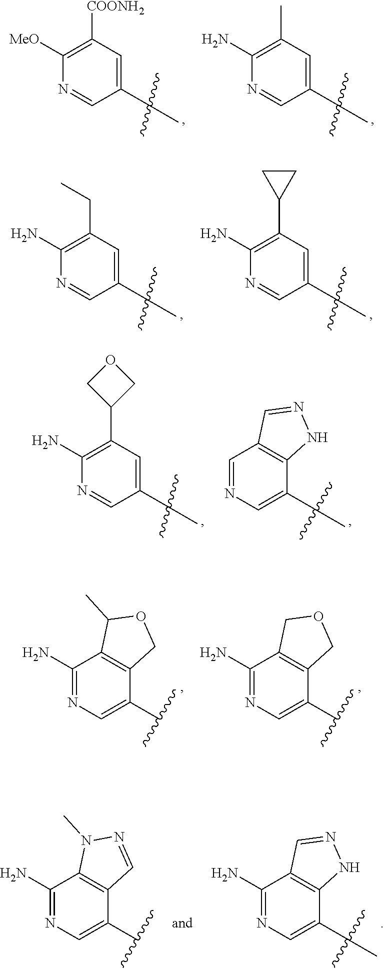

Embodiment 101. The compound of embodiment 40, or a pharmaceutically acceptable salt thereof, wherein Ring A is selected from the group consisting of:

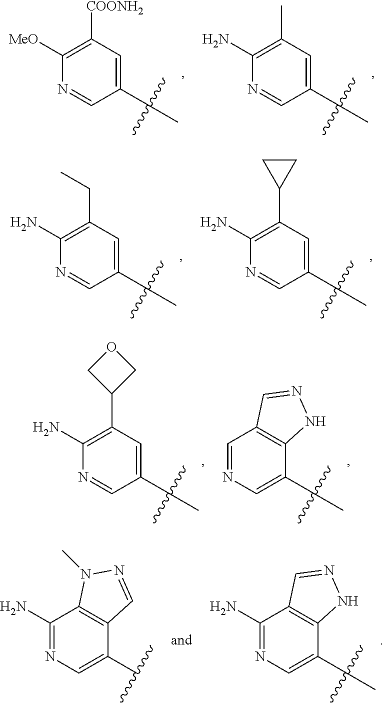

Embodiment 102. The compound of embodiment 40, or a pharmaceutically acceptable salt thereof, wherein Ring A is selected from the group consisting of:

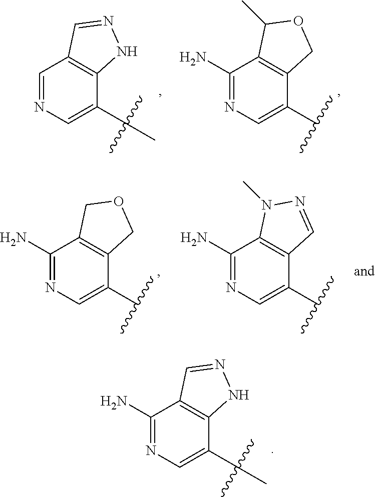 [SMILES: *c1cncc2cn[nH]c12 ; *c1cnc(N)c2c1COC2C ; *c1cnc(N)c2c1COC2 ; *c1cnc(N)c2c1cnn2C ; *c1cnc(N)c2cn[nH]c12]

Embodiment 103. The compound of embodiment 40, or a pharmaceutically acceptable salt thereof, wherein Ring A is selected from the group consisting of:

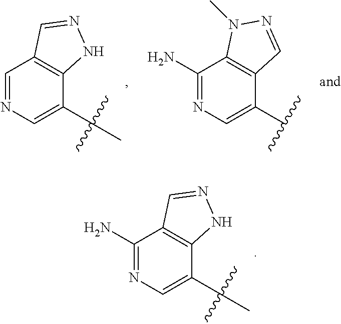 [SMILES: *c1cncc2cn[nH]c12 ; *c1cnc(N)c2c1cnn2C ; *c1cnc(N)c2cn[nH]c12]

Embodiment 104. The compound of embodiment 40, or a pharmaceutically acceptable salt thereof, wherein Ring A is selected from the group consisting of:

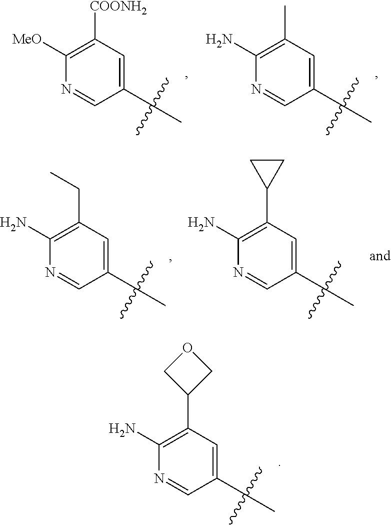

Embodiment 105. The compound of any one of embodiments 40, 41, 43, 45 and 66-78, or a pharmaceutically acceptable salt thereof, wherein Ring A is selected from the group consisting of:

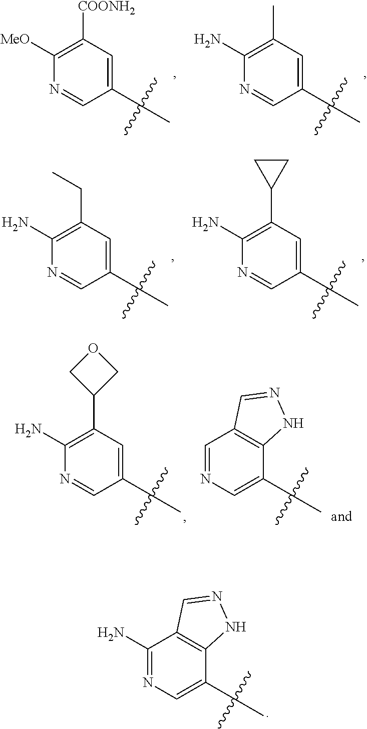

Embodiment 106. The compound of embodiment 40, or a pharmaceutically acceptable salt thereof, wherein Ring A is

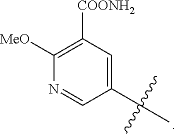

Embodiment 107. The compound of embodiment 40, or a pharmaceutically acceptable salt thereof, wherein Ring A is

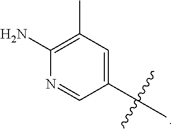 [SMILES: *c1cnc(N)c(C)c1]

Embodiment 108. The compound of embodiment 40, or a pharmaceutically acceptable salt thereof, wherein Ring A is

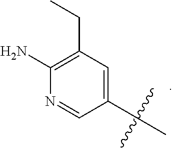 [SMILES: *c1cnc(N)c(CC)c1]

Embodiment 109. The compound of embodiment 40, or a pharmaceutically acceptable salt thereof, wherein Ring A is

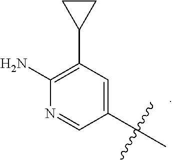 [SMILES: *c1cnc(N)c(C2CC2)c1]

Embodiment 110. The compound of embodiment 40, or a pharmaceutically acceptable salt thereof, wherein Ring A is

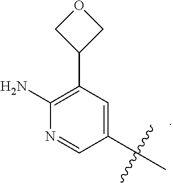 [SMILES: *c1cnc(N)c(C2COC2)c1]

Embodiment 111. The compound of embodiment 40, or a pharmaceutically acceptable salt thereof, wherein Ring A is N

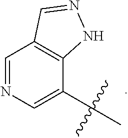 [SMILES: *c1cncc2cn[nH]c12]

Embodiment 112. The compound of embodiment 40, or a pharmaceutically acceptable salt thereof, wherein Ring A is

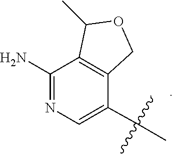 [SMILES: *c1cnc(N)c2c1COC2C]

Embodiment 113. The compound of embodiment 40, or a pharmaceutically acceptable salt thereof, wherein Ring A is

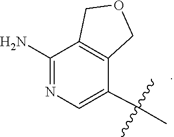 [SMILES: *c1cnc(N)c2c1COC2]

Embodiment 114. The compound of embodiment 40, or a pharmaceutically acceptable salt thereof, wherein Ring A is

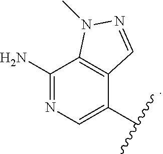 [SMILES: *c1cnc(N)c2c1cnn2C]

Embodiment 115. The compound of embodiment 40, or a pharmaceutically acceptable salt thereof, wherein Ring A is

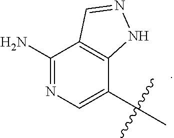 [SMILES: *c1cnc(N)c2cn[nH]c12]

Embodiment 116. The compound of any one of embodiments 1 to 12 and 19 to 115, or a pharmaceutically acceptable salt thereof, wherein each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(=O)₂N(R^{a7})₂, wherein each alkyl, cycloalkyl, heterocyclyl, aryl, heteroaryl, arylalkyl and heteroarylalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂) or a combination thereof.

Embodiment 117. The compound of any one of embodiments 1 to 12 and 19 to 115, or a pharmaceutically acceptable salt thereof, wherein each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, —C₆-C₁₀ aryl, 5-10 membered heteroaryl, arylalkyl, heteroarylalkyl, —C(═O)R^{a7} and —C(═O)OR^{a7}, wherein each alkyl, cycloalkyl, heterocyclyl, aryl, heteroaryl, arylalkyl and heteroarylalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂).

Embodiment 118. The compound of any one of embodiments 1 to 12 and 19 to 115, or a pharmaceutically acceptable salt thereof, wherein each R⁷ is independently selected from the group consisting of —C₁-C₆ alkyl (e.g., -Me, -Et, —Pr, —ⁱPr, -^{tt}Bu, -^{t}Bu, -sec-Bu, -iso-Bu, neopentyl), —C₃-C₉ cycloalkyl (e.g., cyclopropyl, cyclobutyl, cyclopentyl, cyclohexyl), —C(═O)R^{a7} and —C(═O)OR^{a7}, wherein the alkyl and cycloalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂).

Embodiment 119. The compound of any one of embodiments 1 to 12 and 19 to 115, or a pharmaceutically acceptable salt thereof, wherein each R⁷ is independently selected from the group consisting of —C₁-C₆ alkyl (e.g., -Me, -Et, —Pr, —ⁱPr, -^{tt}Bu, -^{t}Bu, -sec-Bu, -iso-Bu, neopentyl), —C(═O)R^{a7} and —C(═O)OR^{a7}, wherein the alkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂).

Embodiment 120. The compound of any one of embodiments 1 to 12 and 19 to 115, or a pharmaceutically acceptable salt thereof, wherein each R⁷ is independently selected from the group consisting of -Me, -Et, —Pr, —ⁱPr, -^{tt}Bu, -^{t}Bu, -sec-Bu, -iso-Bu, neopentyl, cyclopropyl, cyclobutyl, —C(═O)R^{a7} and —C(═O)OR^{a7}, wherein the cyclopropyl and cyclobutyl is substituted at any available position with 0, 1 or 2 instances of -Me.

Embodiment 121. The compound of any one of embodiments 1 to 12 and 19 to 115, or a pharmaceutically acceptable salt thereof, wherein each R⁷ is —C(═O)R^{a7}.

Embodiment 122. The compound of any one of embodiments 1 to 12 and 19 to 115, or a pharmaceutically acceptable salt thereof, wherein each R⁷ is —C(═O)OR^{a7}.

Embodiment 123. The compound of any one of embodiments 1 to 12 and 19 to 122, or a pharmaceutically acceptable salt thereof, wherein each R^{a7} is independently selected from the group consisting of H, —C₁-C₆ alkyl (e.g., -Me, -Et, —Pr, —ⁱPr, -^{tt}Bu, -^{t}Bu, -sec-Bu, -iso-Bu), C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl), 3-7 membered heterocyclyl (e.g., azetidinyl, oxetanyl, piperidyl, pyrrolidinyl, tetrahydrofuranyl, tetrahydropyranyl), cycloalkylalkyl (e.g., —CH₂-cyclopropyl, —CH₂-cyclobutyl, —CH₂-cyclopentyl, —CH₂-cyclohexyl, —CH₂-cycloheptyl), heterocyclylalkyl (e.g., —CH₂-azetidinyl, —CH₂-pyrrolidinyl, —CH₂-piperidinyl, —CH₂-tetrahydrofuranyl, —CH₂-tetrahydropyranyl), aryl (e.g., phenyl), 5-6 membered heteroaryl (e.g., pyridinyl, pyrimidinyl, pyrazinyl, thiophenyl, furyl, thiazolyl, imidazolyl, pyrazolyl), arylalkyl (e.g., benzyl) and heteroarylalkyl ((e.g., —CH₂-pyridinyl, —CH₂-pyrimidinyl, —CH₂-pyrazinyl, —CH₂-thiophenyl, —CH₂-furyl, —CH₂-thiazolyl, —CH₂-imidazolyl, —CH₂-pyrazolyl), wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R is independently selected from the group consisting of ═O, halo, —CN, —C₁-C₆ alkyl, —C₁-C₆ heteroalkyl, —C₁-C₆ hydroxyalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR, —NR^{b}C(═O)R^{b}, —NR^{b}C(═O)OR, —C(═O)N(R^{b})₂, —OC(═O)N(R^{b})₂, —S(═O)R, —S(═O)₂R, —SR^{b}, —S(═O)(═NR^{b})R, —NR^{b}S(═O)₂R and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -Bu, -sec-Bu, -iso-Bu).and C₃-C₉ cycloalkyl (e.g., cyclopropyl, cyclobutyl, cyclopentyl, cyclohexyl)).

Embodiment 124. The compound of any one of embodiments 1 to 12 and 19 to 122, or a pharmaceutically acceptable salt thereof, wherein each R^{a7} is independently selected from the group consisting of H, —C₁-C₆ alkyl (e.g., -Me, -Et, —Pr, —ⁱPr, -^{tt}Bu, -^{t}Bu, -sec-Bu, -iso-Bu), C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl), cycloalkylalkyl (e.g., —CH₂-cyclopropyl, —CH₂-cyclobutyl, —CH₂-cyclopentyl, —CH₂-cyclohexyl, —CH₂-cycloheptyl), wherein each alkyl, cycloalkyl, and cycloalkylalkyl is substituted at any available position with 0, 1, 2 or 3 instances of R⁵, wherein each R⁵ is independently selected from the group consisting of ═O, halo (e.g., —F, —C₁), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃).

Embodiment 125. The compound of any one of embodiments 1 to 12 and 19 to 122, or a pharmaceutically acceptable salt thereof, wherein each R^{a7} is independently —C₁-C₆ alkyl (e.g., -Me, -Et, —Pr, —ⁱPr, -^{tt}Bu, -^{t}Bu, -sec-Bu, -iso-Bu) substituted with 0, 1, 2 or 3 instances of R⁵, wherein each R is independently selected from the group consisting of halo (e.g., —F, —Cl), —CN, —C₁-C₆ haloalkyl (e.g., —CF₃, —CH₂CF₃, —CF₂CH₃, —CHF₂, —CH₂F) 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), and —OH.

Embodiment 126. The compound of any one of embodiments 1 to 12 and 19 to 122, or a pharmaceutically acceptable salt thereof, wherein each R^{a7} is independently C₃-C₉ cycloalkyl (e.g., cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl) substituted at any available position with 0, 1, 2 or 3 instances of R⁵, wherein each R is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F) and —OH.

Embodiment 127. The compound of any one of embodiments 1 to 12 and 19 to 122, or a pharmaceutically acceptable salt thereof, wherein each R^{a7} is independently —CH₂-cyclopropyl substituted at any available position with 0, 1, 2 or 3 instances of R⁵, wherein each R is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F) and —OH.

Embodiment 128. The compound of any one of embodiments 1 to 12 and 19 to 122, or a pharmaceutically acceptable salt thereof, wherein each R^{a7} is independently cyclopropyl substituted at any available position with 0, 1, 2 or 3 instances of R⁵, wherein each R⁵ is independently selected from the group consisting of halo (e.g., —F, —Cl), —CN, —C₁-C₆ alkyl (e.g., -Me, -Et, iPr), —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F) and —OH.

Embodiment 129. The compound of any one of embodiments 1 to 12 and 19 to 128, or a pharmaceutically acceptable salt thereof, wherein each R is independently selected from the group consisting of —F, —CN, -Me, -Et, —CH₂OH, —OH, —CF₃, —CH₂CF₃, —CF₂CH₃, —CHF₂, —CH₂F, —N(CH₃)₂, N-Me-piperazinyl and 1,2,3,4-tetrahydropyrrolo[1,2-a]pyrazin-2-yl.

Embodiment 130. The compound of any one of embodiments 1 to 12 and 19 to 128, or a pharmaceutically acceptable salt thereof, wherein each R is independently selected from the group consisting of -Me, —CF₃, —N(CH₃)₂, N-Me-piperazinyl and 1,2,3,4-tetrahydropyrrolo[1,2-a]pyrazin-2-yl.

Embodiment 131. The compound of any one of embodiments 1 to 12 and 19 to 122, or a pharmaceutically acceptable salt thereof, wherein each R^{a7} is independently selected from the group consisting of -Me, -Et, —Pr, —ⁱPr, -^{tt}Bu, -^{t}Bu, -sec-Bu, -iso-Bu, cyclopropyl, cyclobutyl, cyclopentyl, cyclohexyl, cycloheptyl, bicyclo[1.1.1]pentyl, spiro[2.2]pentyl, bicyclo[2.1.0]pentyl, bicyclo[3.1.0]hexyl, —CH₂-cyclopropyl, —CH₂-cyclobutyl, —CH₂-cyclopentyl, —CH₂-cyclohexyl, each substituted at any available position with 0, 1, 2 or 3 instances of R⁵, wherein each R⁵ is independently selected from the group consisting of F, —Cl—CN, -Me, -Et, iPr —CH₂OH, —CF₃, —CH₂CF₃, —CF₂CH₃, —CHF₂, —CH₂F, —OH, —OCH₃, —N(CH₃)₂, N-Me-piperazinyl and 1,2,3,4-tetrahydropyrrolo[1,2-a]pyrazin-2-yl.

Embodiment 132. The compound of any one of embodiments 1 to 12 and 19 to 122, or a pharmaceutically acceptable salt thereof, wherein each R^{a7} is independently selected from the group consisting of -Me, -Et, —ⁱPr, -^{t}Bu, -iso-Bu, cyclopropyl, —CH₂-cyclopropyl, each substituted at any available position with 0, 1, 2 or 3 instances of R, wherein each R is independently selected from the group consisting of Me, —CF₃, —N(CH₃)₂, N-Me-piperazinyl and 1,2,3,4-tetrahydropyrrolo[1,2-a]pyrazin-2-yl.

Embodiment 133. The compound of any one of embodiments 1 to 12 and 19 to 115, or a pharmaceutically acceptable salt thereof, wherein each R⁷ is independently C₁-C₆ alkyl (e.g., -Me, -Et, —Pr, —ⁱPr, -^{tt}Bu, -^{t}Bu, -sec-Bu, -iso-Bu, neopentyl).

Embodiment 134. The compound of any one of embodiments 1 to 12 and 19 to 115, or a pharmaceutically acceptable salt thereof, wherein each R⁷ is independently selected from the group consisting of -Me, -Et, —ⁱPr, -iso-Bu, neopentyl.

Embodiment 135. The compound of any one of embodiments 1 to 12 and 19 to 115, or a pharmaceutically acceptable salt thereof, wherein each R⁵ is independently selected from the group consisting of -Me, —ⁱPr, -iso-Bu, neopentyl.

Embodiment 136. The compound of any one of embodiments 1 to 12 and 19 to 115, or a pharmaceutically acceptable salt thereof, wherein each R⁷ is independently selected from the group consisting of:

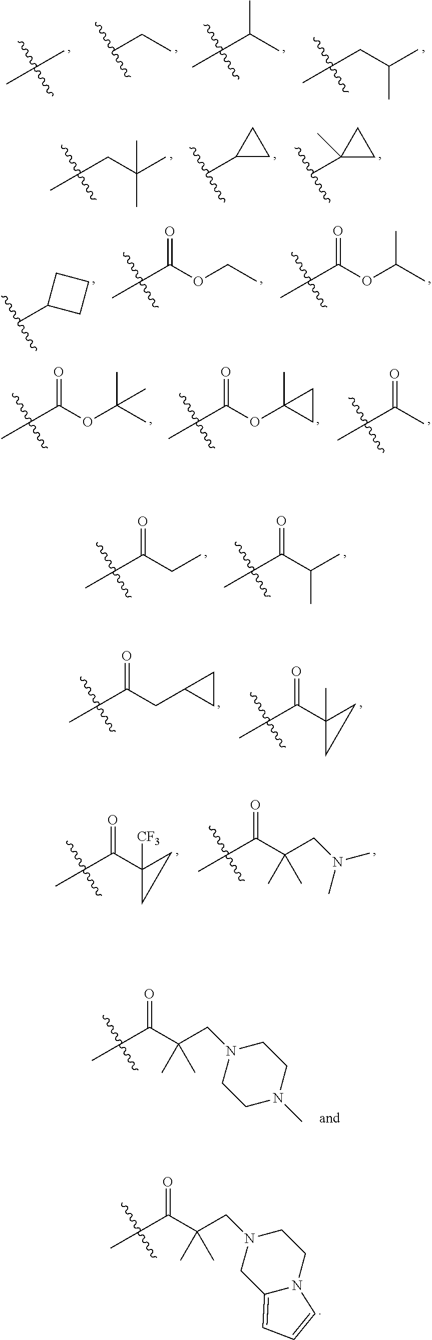

Embodiment 137. The compound of any one of embodiments 1 to 12 and 19 to 115, or a pharmaceutically acceptable salt thereof, wherein each R⁷ is independently selected from the group consisting of:

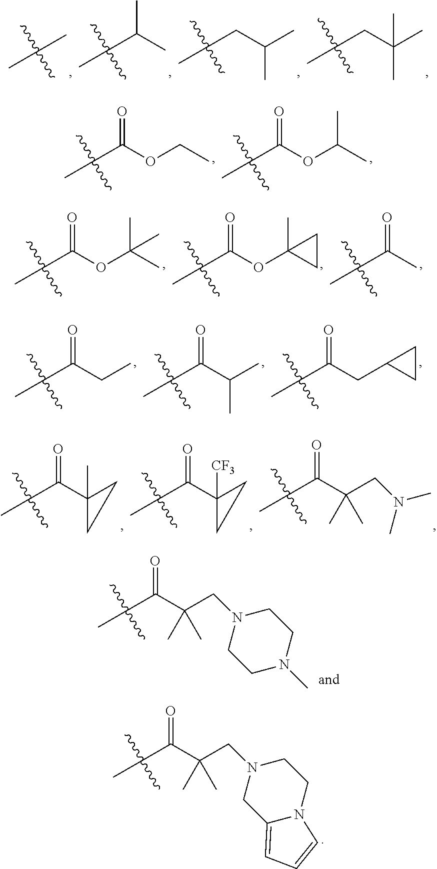

Embodiment 138. The compound of any one of embodiments 1 to 12 and 19 to 115, or a pharmaceutically acceptable salt thereof, wherein R⁷ is

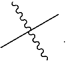

Embodiment 139. The compound of any one of embodiments 1 to 12 and 19 to 115, or a pharmaceutically acceptable salt thereof, wherein R⁷ is

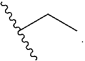 [SMILES: *CC]

Embodiment 140. The compound of any one of embodiments 1 to 12 and 19 to 115, or a pharmaceutically acceptable salt thereof, wherein R⁷ is

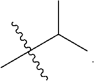 [SMILES: *C(C)C]

Embodiment 141. The compound of any one of embodiments 1 to 12 and 19 to 115, or a pharmaceutically acceptable salt thereof, wherein R⁷ is

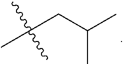 [SMILES: *CC(C)C]

Embodiment 142. The compound of any one of embodiments 1 to 12 and 19 to 115, or a pharmaceutically acceptable salt thereof, wherein R⁷ is

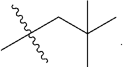 [SMILES: *CC(C)(C)C]

Embodiment 143. The compound of any one of embodiments 1 to 12 and 19 to 115, or a pharmaceutically acceptable salt thereof, wherein R⁷ is

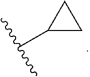 [SMILES: *C1CC1]

Embodiment 144. The compound of any one of embodiments 1 to 12 and 19 to 115, or a pharmaceutically acceptable salt thereof, wherein R⁷ is

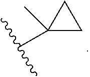 [SMILES: *C1(C)CC1]

Embodiment 145. The compound of any one of embodiments 1 to 12 and 19 to 115, or a pharmaceutically acceptable salt thereof, wherein R is

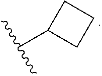 [SMILES: *C1CCC1]

Embodiment 146. The compound of any one of embodiments 1 to 12 and 19 to 115, or a pharmaceutically acceptable salt thereof, wherein R⁷ is

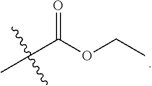 [SMILES: *C(=O)OCC]

Embodiment 147. The compound of any one of embodiments 1 to 12 and 19 to 115, or a pharmaceutically acceptable salt thereof, wherein R⁷ is

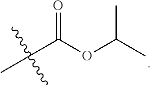 [SMILES: *C(=O)OC(C)C]

Embodiment 148. The compound of any one of embodiments 1 to 12 and 19 to 115, or a pharmaceutically acceptable salt thereof, wherein R⁷ is

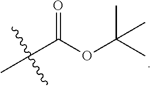 [SMILES: *C(=O)OC(C)(C)C]

Embodiment 149. The compound of any one of embodiments 1 to 12 and 19 to 115, or a pharmaceutically acceptable salt thereof, wherein R⁷ is

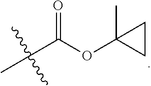 [SMILES: *C(=O)OC1(C)CC1]

Embodiment 150. The compound of any one of embodiments 1 to 12 and 19 to 115, or a pharmaceutically acceptable salt thereof, wherein R⁷ is

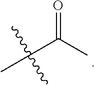 [SMILES: *C(C)=O]

Embodiment 151. The compound of any one of embodiments 1 to 12 and 19 to 115, or a pharmaceutically acceptable salt thereof, wherein R⁷ is

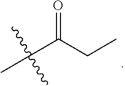 [SMILES: *C(=O)CC]

Embodiment 152. The compound of any one of embodiments 1 to 12 and 19 to 115, or a pharmaceutically acceptable salt thereof, wherein R⁷ is

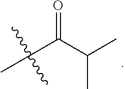 [SMILES: *C(=O)C(C)C]

Embodiment 153. The compound of any one of embodiments 1 to 12 and 19 to 115, or a pharmaceutically acceptable salt thereof, wherein R⁷ is

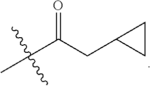 [SMILES: *C(=O)CC1CC1]

Embodiment 154. The compound of any one of embodiments 1 to 12 and 19 to 115, or a pharmaceutically acceptable salt thereof, wherein R⁷ is

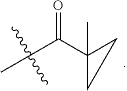 [SMILES: *C(=O)C1(C)CC1]

Embodiment 155. The compound of any one of embodiments 1 to 12 and 19 to 115, or a pharmaceutically acceptable salt thereof, wherein R⁷ is

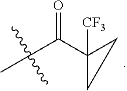 [SMILES: *C(=O)C1(C(F)(F)F)CC1]

Embodiment 156. The compound of any one of embodiments 1 to 12 and 19 to 115, or a pharmaceutically acceptable salt thereof, wherein R⁷ is

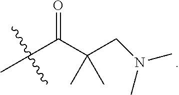 [SMILES: *C(=O)C(C)(C)CN(C)C]

Embodiment 157. The compound of any one of embodiments 1 to 12 and 19 to 115, or a pharmaceutically acceptable salt thereof, wherein R⁷ is

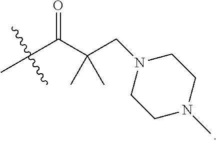 [SMILES: *C(=O)C(C)(C)CN1CCN(C)CC1]

Embodiment 158. The compound of any one of embodiments 1 to 12 and 19 to 115, or a pharmaceutically acceptable salt thereof, wherein R⁷ is

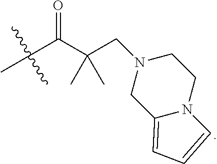 [SMILES: *C(=O)C(C)(C)CN1CCn2cccc2C1]

Embodiment 159. The compound of any one of embodiments 1 to 158, or a pharmaceutically acceptable salt thereof, wherein Ring B is selected from the group consisting of C₆-C₁₀ aryl (e.g., phenyl, naphthalenyl), 5-6 membered monocyclic heteroaryl, and 8-10 membered bicyclic heteroaryl, each optionally substituted at any available position.

Embodiment 160. The compound of any one of embodiments 1 to 158, or a pharmaceutically acceptable salt thereof, wherein Ring B is selected from the group consisting of C₆-C₁₀ aryl and 8-10 membered bicyclic heteroaryl wherein the aryl and heteroaryl are optionally substituted at any available position.

Embodiment 161. The compound of any one of embodiments 1 to 158, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, triazolo[4,3-a]pyridinyl, imidazo[1,2-a]pyridinyl, 1H-pyrazolo[4,3-b]pyridinyl),1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each optionally substituted.

Embodiment 162. The compound of any one of embodiments 1 to 158, or a pharmaceutically acceptable salt thereof, wherein Ring B is selected from the group consisting of thiophenyl, phenyl and benzo[d]thiazolyl, each optionally substituted.

Embodiment 163. The compound of any one of embodiments 1 to 158, or a pharmaceutically acceptable salt thereof, wherein Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each optionally substituted.

Embodiment 164. The compound of any one of embodiments 1 to 158, or a pharmaceutically acceptable salt thereof, wherein Ring B is selected from the group consisting of thiophen-2-yl, thiophen-3-yl, phenyl, benzo[d]thiazol-5-yl, each optionally substituted.

Embodiment 165. The compound of any one of embodiments 1 to 158, or a pharmaceutically acceptable salt thereof, wherein Ring B is C₆-C₁₀ aryl (e.g., naphthalenyl, phenyl), wherein the aryl is optionally substituted at any available position.

Embodiment 166. The compound of any one of embodiments 1 to 158, or a pharmaceutically acceptable salt thereof, wherein Ring B is naphthalenyl or phenyl, each optionally substituted at any available position.

Embodiment 167. The compound of any one of embodiments 1 to 158, or a pharmaceutically acceptable salt thereof, wherein Ring B is phenyl, optionally substituted at any available position.

Embodiment 168. The compound of any one of embodiments 1 to 158, or a pharmaceutically acceptable salt thereof, wherein Ring B is 5-6 membered monocyclic heteroaryl wherein the heteroaryl is optionally substituted at any available position.

Embodiment 169. The compound of any one of embodiments 1 to 158, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, pyridinyl, pyrimidinyl, pyrazinyl and pyridazinyl, each optionally substituted.

Embodiment 170. The compound of any one of embodiments 1 to 158, or a pharmaceutically acceptable salt thereof, wherein Ring B is thiophenyl, optionally substituted.

Embodiment 171. The compound of any one of embodiments 1 to 158, or a pharmaceutically acceptable salt thereof, wherein Ring B is selected from the group consisting of pyrazol-5-yl, thiophene-2-yl, thiophen-3-yl, oxazol-5-yl, thiazol-5-yl, pyridin-3-yl and pyridin-4-yl, each optionally substituted.

Embodiment 172. The compound of any one of embodiments 1 to 158, or a pharmaceutically acceptable salt thereof, wherein Ring B is selected from the group consisting of thiophen-2-yl and thiophen-3-yl, each optionally substituted.

Embodiment 173. The compound of any one of embodiments 1 to 158, or a pharmaceutically acceptable salt thereof, wherein Ring B is an 8-10 membered bicyclic heteroaryl, wherein the bicyclic heteroaryl is optionally substituted at any available position.

Embodiment 174. The compound of any one of embodiments 1 to 158, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl,

triazolo[4,3-a]pyridinyl, imidazo[1,2-a]pyridinyl, 1H-pyrazolo[4,3-b]pyridinyl),1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each optionally substituted.

Embodiment 175. The compound of any one of embodiments 1 to 158, or a pharmaceutically acceptable salt thereof, wherein Ring B is benzo[d]thiazolyl, optionally substituted.

Embodiment 176. The compound of any one of embodiments 1 to 158, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each optionally substituted.

Embodiment 177. The compound of any one of embodiments 1 to 158, or a pharmaceutically acceptable salt thereof, wherein Ring B is benzo[d]thiazol-5-yl, optionally substituted.

Embodiment 178. The compound of any one of embodiments 1 to 177 wherein each Ring B is substituted at any available position with 0, 1, 2 or 3 instances of R³, wherein:

each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3},—SR^{a3}, —S(═O)(═NR^{a3})R^{a3}, —NR^{a3}S(═O)₂R^{a3} and —S(═O)₂N(R^{a3})₂, wherein each alkyl, cycloalkyl, heterocyclyl, aryl, heteroaryl, cycloalkylalkyl, heterocyclylalkyl, arylalkyl and heteroarylalkyl of R³ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof);

each R^{a3} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

Embodiment 179. The compound of embodiment 178, or a pharmaceutically acceptable salt thereof, wherein Ring B is selected from the group consisting of C₆-C₁₀ aryl (e.g., phenyl, naphthalenyl), 5-6 membered monocyclic heteroaryl, and 8-10 membered bicyclic heteroaryl, each substituted at any available position with 0, 1, 2 or 3 instances of R³.

Embodiment 180. The compound of embodiment 178, or a pharmaceutically acceptable salt thereof, wherein Ring B is selected from the group consisting of C₆-C₁₀ aryl and 8-10 membered bicyclic heteroaryl wherein the aryl and heteroaryl are substituted at any available position with 0, 1, 2 or 3 instances of R³.

Embodiment 181. The compound of embodiment 178, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, phenyl, naphthalenyl, pyridinyl, pyrimidinyl, pyrazinyl, pyridazinyl, indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl),1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl), each substituted at any available position with 0, 1, 2 or 3 instances of R³.

Embodiment 182. The compound of embodiment 178, or a pharmaceutically acceptable salt thereof, wherein Ring B is selected from the group consisting of thiophenyl, phenyl and benzo[d]thiazolyl, each substituted at any available position with 0, 1, 2 or 3 instances of R.

Embodiment 183. The compound of embodiment 178, or a pharmaceutically acceptable salt thereof, wherein Ring B is selected from the group consisting of pyrazol-5-yl, thiophen-2-yl, thiophen-3-yl, oxazol-5-yl, thiazol-5-yl, pyridin-3-yl, pyridin-4-yl, phenyl, naphthalen-1-yl, naphthalen-2-yl, indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl,

triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl), each substituted at any available position with 0, 1, 2 or 3 instances of R.

Embodiment 184. The compound of embodiment 178, or a pharmaceutically acceptable salt thereof, wherein Ring B is selected from the group consisting of thiophen-2-yl, thiophen-3-yl, phenyl and benzo[d]thiazol-5-yl, each substituted at any available position with 0, 1, 2 or 3 instances of R.

Embodiment 185. The compound of embodiment 178, or a pharmaceutically acceptable salt thereof, wherein Ring B is C₆-C₁₀ aryl (e.g., naphthalenyl, phenyl), wherein the aryl is substituted at any available position with 0, 1, 2 or 3 instances of R³.

Embodiment 186. The compound of embodiment 178, or a pharmaceutically acceptable salt thereof, wherein Ring B is naphthalenyl or phenyl, each substituted at any available position with 0, 1, 2 or 3 instances of R.

Embodiment 187. The compound of embodiment 178, or a pharmaceutically acceptable salt thereof, wherein Ring B is phenyl, substituted at any available position with 0, 1, 2 or 3 instances of R³.

Embodiment 188. The compound of embodiment 178, or a pharmaceutically acceptable salt thereof, wherein Ring B is 5-6 membered monocyclic heteroaryl wherein the heteroaryl is substituted at any available position with 0, 1, 2 or 3 instances of R.

Embodiment 189. The compound of embodiment 178, or a pharmaceutically acceptable salt thereof, wherein Ring B is selected from the group consisting of thiophenyl, oxazolyl, isoxazolyl, thiazolyl, isothiazolyl, furanyl, pyrrolyl, pyrazolyl, imidazolyl, triazolyl, pyridinyl, pyrimidinyl, pyrazinyl and pyridazinyl each substituted at any available position with 0, 1, 2 or 3 instances of R.

Embodiment 190. The compound of embodiment 178, or a pharmaceutically acceptable salt thereof, wherein Ring B is thiophenyl, substituted at any available position with 0, 1, 2 or 3 instances of R.

Embodiment 191. The compound of embodiment 178, or a pharmaceutically acceptable salt thereof, wherein Ring B is selected from the group consisting of pyrazol-5-yl, thiophene-2-yl, thiophen-3-yl, oxazol-5-yl, thiazol-5-yl, pyridin-3-yl and pyridin-4-yl, substituted at any available position with 0, 1, 2 or 3 instances of R.

Embodiment 192. The compound of embodiment 178, or a pharmaceutically acceptable salt thereof, wherein Ring B is selected from the group consisting of thiophen-2-yl and thiophen-3-yl, each substituted at any available position with 0, 1, 2 or 3 instances of R.

Embodiment 193. The compound of embodiment 178, or a pharmaceutically acceptable salt thereof, wherein Ring B is an 8-10 membered bicyclic heteroaryl, wherein the bicyclic heteroaryl is substituted at any available position with 0, 1, 2 or 3 instances of R.

Embodiment 194. The compound of embodiment 178, or a pharmaceutically acceptable salt thereof, wherein Ring B is selected from the group consisting of indolyl, benzofuranyl, 1H-indazolyl, 2H-indazolyl, benzo[b]thiophenyl, quinolinyl, 1,5-naphthyridinyl, 1,2-dihydro-1,5-naphthyridinyl, 1,2,3,4-tetrahydro-1,8-naphthyridinyl, imidazo[1,2-a]pyridinyl, imidazo[1,5-a]pyridinyl, isoquinolinyl, benzo[d]imidazolyl, benzo[d]thiazolyl, benzo[d]isothiazolyl, benzo[d]oxazolyl, [1,2,4]triazolo[4,3-a]pyridinyl, imidazo[1,2-a]pyridinyl, 1H-pyrazolo[4,3-b]pyridinyl),1H-pyrazolo[3,4-b]pyridinyl, 1H-thieno[2,3-c]pyrazolyl, 1H-thieno[3,2-c]pyrazolyl, thiazolo[5,4-b]pyridinyl and 1,2,3,4-tetrahydro-1,8-naphthyridinyl, each substituted at any available position with 0, 1, 2 or 3 instances of R.

Embodiment 195. The compound of embodiment 178, or a pharmaceutically acceptable salt thereof, wherein Ring B is benzo[d]thiazolyl, substituted at any available position with 0, 1, 2 or 3 instances of R³.

Embodiment 196. The compound of embodiment 178, or a pharmaceutically acceptable salt thereof, wherein Ring B is selected from the group consisting of indol-4-yl, indol-5-yl, benzofuran-5-yl, benzofuran-6-yl, 1H indazol-5-yl, 1H indazol-4-yl, 2H-indazol-6-yl, 2H-indazol-5-yl, benzo[b]thiophen-3-yl, benzo[b]thiophen-5-yl, quinolin-6-yl, quinolin-7-yl, quinoline-3-yl, isoquinolin-6-yl, benzo[d]imidazo-5-yl, 1H-benzo[d]imidazol-4-yl, benzo[d]thiazol-5-yl, benzo[d]thiazol-6-yl, benzo[d]thiazol-4-yl, benzo[d]isothiazol-5-yl, benzo[d]oxazol-4-yl, benzo[d]oxazol-5-yl, [1,2,4]triazolo[4,3-a]pyridin-6-yl, imidazo[1,2-a]pyridin-6-yl, imidazo[1,2-a]pyridin-7-yl, imidazo[1,5-a]pyridin-6-yl, pyrazolo[4,3-b]pyridin-6-yl, 1H-pyrazolo[3,4-b]pyridin-5-yl, 1H-pyrazolo[3,4-b]pyridin-5-yl, 1H-pyrazolo[4,3-b]pyridin-5-yl, 1H-pyrazolo[4,3-b]pyridin-6-yl, 1H-thieno[2,3-c]pyrazol-5-yl, 1H-thieno[3,2-c]pyrazol-5-yl and thiazolo[5,4-b]pyridin-6-yl, each substituted at any available position with 0, 1, 2 or 3 instances of R³.

Embodiment 197. The compound of embodiment 178, or a pharmaceutically acceptable salt thereof, wherein Ring B is benzo[d]thiazol-5-yl, substituted at any available position with 0, 1, 2 or 3 instances of R³.

Embodiment 198. The compound of embodiment 178, or a pharmaceutically acceptable salt thereof, wherein Ring B is selected from the group consisting of:

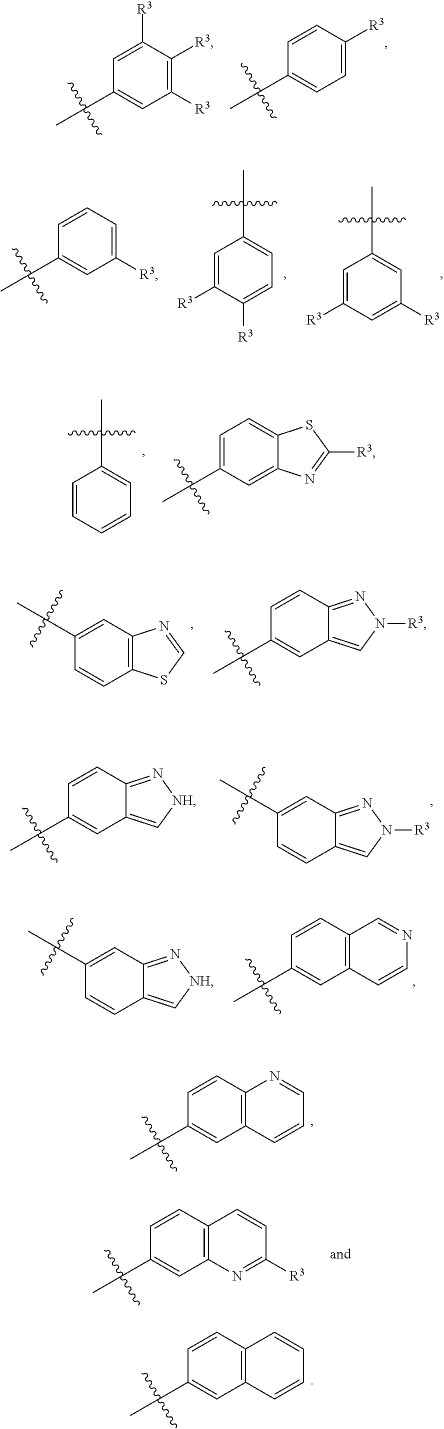

Embodiment 199. The compound of embodiment 178, or a pharmaceutically acceptable salt thereof, wherein Ring B is selected from the group consisting of:

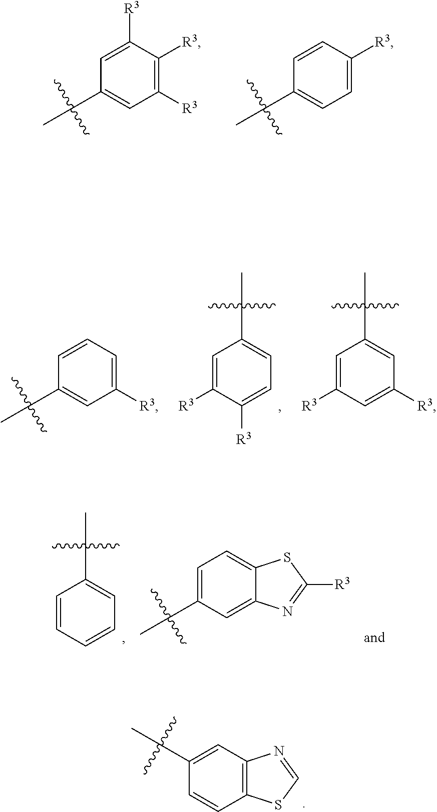

Embodiment 200. The compound of embodiment 178, or a pharmaceutically acceptable salt thereof, wherein Ring B is selected from the group consisting of:

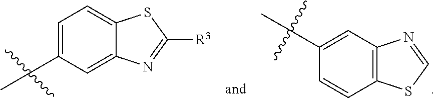

Embodiment 201. The compound of embodiment 178, or a pharmaceutically acceptable salt thereof, wherein Ring B is selected from the group consisting of:

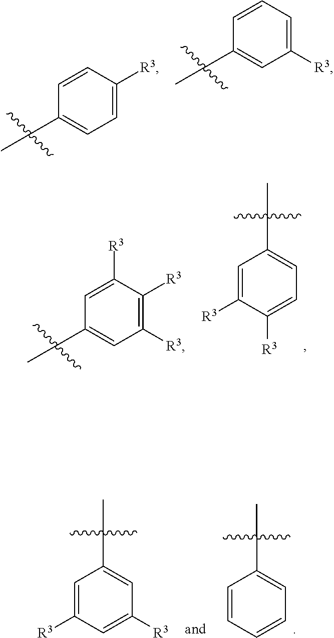

Embodiment 202. The compound of embodiment 178, or a pharmaceutically acceptable salt thereof, wherein Ring B is

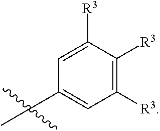

Embodiment 203. The compound of embodiment 178, or a pharmaceutically acceptable salt thereof, wherein Ring B is

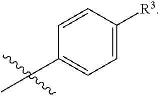

Embodiment 204. The compound of embodiment 178, or a pharmaceutically acceptable salt thereof, wherein Ring B is

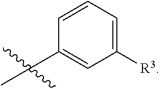

Embodiment 205. The compound of embodiment 178, or a pharmaceutically acceptable salt thereof, wherein Ring B is

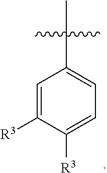

Embodiment 206. The compound of embodiment 178, or a pharmaceutically acceptable salt thereof, wherein Ring B is

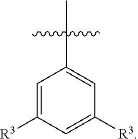

Embodiment 207. The compound of embodiment 178, or a pharmaceutically acceptable salt thereof, wherein Ring B is

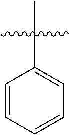 [SMILES: *c1ccccc1]

Embodiment 208. The compound of embodiment 178, or a pharmaceutically acceptable salt thereof, wherein Ring B is

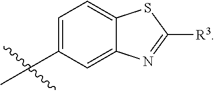

Embodiment 209. The compound of embodiment 178, or a pharmaceutically acceptable salt thereof, wherein Ring B is

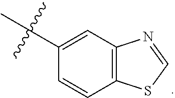 [SMILES: *c1ccc2scnc2c1]

Embodiment 210. The compound of any one of embodiments 1 to 209, or a pharmaceutically acceptable salt thereof, wherein each R³ is independently selected from the group consisting of D, ═O, halo (e.g., —F, —Cl, —Br), —CN, —C₁-C₆ alkyl (e.g., -Me, -Et, —Pr, —ⁱPr, -sec-Bu, -Bu), —C₁-C₆ heteroalkyl (e.g., —CH₂OH, —CH(OH)(CH₃),—C(OH)(CH₃)₂, —CH₂NH₂), —C₁-C₆ haloalkyl (e.g., —CHF₂, —CH₂CF₃, —CF₃), —C₃-C₉ cycloalkyl(e.g., cyclopropyl, cyclobutyl, cyclopentyl, cyclohexyl), 3-10 membered heterocyclyl (e.g., oxetanyl, pyrrolidinyl, piperidinyl, piperazinyl), 5-10 membered heteroaryl (e.g., pyrazolyl, thiazolyl, thiophenyl, pyridinyl), cycloalkylalkyl (e.g. —CH₂-cyclopropyl), heterocyclylalkyl (e.g., —CH₂-morpholinyl), heteroarylalkyl (e.g., —CH₂-triazolyl, —CH₂-imidazolyl, —CH₂-pyrazolyl), —OR^{a3} (e.g., —OH, —OCH₃, —O-tetrahydrofuranyl, —O-tetrahydropyran-4-yl, —OCF₃, —OCHF₂), —N(R^{a3})₂, (e.g., —NH₂, —NHR^{a3}, —NHCH₃, —N(CH₃)₂, —NHCH₂CF₃, —NH-oxetan-3-yl, —NH—(N-Me-2-oxo-pyrrolidin-3-yl), —NR^{a3}C(═O)R^{a3} (e.g., —NHC(═O)Me), —C(═O)N(R^{a3})₂, (e.g., —C(═O)NH₂, —C(═O)NHCH₃), —OC(═O)R^{a3} (e.g., —OC(═O)Me), —S(═O)R^{a3} (e.g., —SO₂Me), —NR^{a3}S(═O)₂R^{a3} (e.g., —NHSO₂Me) and —S(═O)₂N(R^{a3})₂ (e.g., —SO₂NH₂, —SO₂NHCH₃), wherein each alkyl, cycloalkyl, heterocyclyl, aryl, heteroaryl, cycloalkylalkyl, heterocyclylalkyl, arylalkyl and heteroarylalkyl is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof); and

each R^{a3} is independently selected from the group consisting of H, —C₁-C₆ alkyl, (e.g., -Me, -Et, —Pr, —ⁱPr, -sec-Bu, -^{t}Bu), —C₁-C₆ haloalkyl (e.g., —CF₃, —CHF₂, —CH₂CF₃), —C₁-C₆ heteroalkyl substituted with 0 or 1 instances of ═O (e.g., —CH₂CH₂N(CH₃)₂, —CH₂C(═O)N(CH₃)₂, —CH(CH₃)CH₂N(CH₃)₂, —CH(CH₃)C(═O)N(CH₃)₂), C₃-C₉ cycloalkyl and 3-10 membered heterocyclyl substituted with 0 or 1 instances of ═O, -Me or a combination thereof (e.g. tetrahydrofuran-3-yl, tetrahydropyran-4-yl, oxetan-3-yl, N-Me-2-oxo-pyrrolidin-3-yl).

Embodiment 211. The compound of any one of embodiments 1 to 210, or a pharmaceutically acceptable salt thereof, wherein each R^{a3} is independently selected from the group consisting of H, -Me, -Et, —Pr, —ⁱPr, -sec-Bu, -^{t}Bu, —CF₃, —CHF₂, —CH₂CF₃, —CH₂CH₂N(CH₃)₂, —CH₂C(═O)N(CH₃)₂, —CH(CH₃)CH₂N(CH₃)₂, —CH(CH₃)C(═O)N(CH₃)₂), tetrahydrofuran-3-yl, tetrahydropyran-4-yl, oxetan-4-yl and N-Me-2-oxo-pyrrolidin-3-yl.

Embodiment 212. The compound of any one of embodiments 1 to 210, or a pharmaceutically acceptable salt thereof, wherein each R^{a3} is independently, —CH₂CH₂N(CH₃)₂.

Embodiment 213. The compound of any one of embodiments 1 to 209, or a pharmaceutically acceptable salt thereof, wherein each R³ is independently selected from the group consisting of -D, ═O, —F, —Cl, —Br, —CN, -Me, -Et, —Pr, —ⁱPr, -sec-Bu, -—Bu, —CHF₂, —CH₂CF₃, —CF₃, —CH₂OH, —CH(OH)(CH₃),—C(OH)(CH₃)₂, —CH₂NH₂, cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiazol-5-yl,thiophen-2-yl, —CH₂-cyclopropyl, —CH₂-morpholin-4-yl, —CH₂₋₁,2,4-triazol-1-yl, —CH₂-imidazol-1-yl, —CH₂-pyrazol-1-yl, —OH, —OCH₃, —OCF₃, —OCHF₂, —O-tetrahydrofuran-3-yl, —O-tetrahydropyran-4-yl, —O—(N-Me-2-oxo-pyrrolidin-3-yl), —OCF₃, —OCHF₂, —NH₂, —NHCH₃, —NHCH₂CF₃, —NH-oxetan-3-yl, —NH—(N-Me-2-oxo-pyrrolidin-3-yl), —N(CH₃)₂, —NHC(═O)Me, —NHCH₂C(═O)N(CH₃)₂, —NHCH(CH₃)C(═O)N(CH₃)₂, —C(═O)NH₂, —C(═O)NHCH₃, —OC(═O)Me, —SO₂Me, —NHSO₂Me,- SO₂NH₂ and —SO₂NHCH₃, wherein each cyclopropyl, cyclobutyl, cyclopentyl, cyclohexyl, pyrrolidin-1-yl, piperidin-4-yl, piperazin-4-yl, pyridin-4-yl, pyrazol-1-yl, pyrazol-3-yl, pyrazol-4-yl, pyrazol-5-yl, thiazol-2-yl, thiophen-2-yl, —CH₂-cyclopropyl, —CH₂-morpholin-4-yl, —CH₂₋₁,2,4-triazol-1-yl —CH₂-imidazol-1-yl and —CH₂-pyrazol-1-yl, can be independently substituted with 0, 1, 2 or 3 instances of -Me, —OH, —C(═O)CH₃, —C(═O)NHCH₃, —NH₂, —NHC(═O)CH₃ or a combination thereof.

Embodiment 214. The compound of any one of embodiments 1 to 209, or a pharmaceutically acceptable salt thereof wherein each R³ is independently selected from the group consisting of -D, ═O, —F, —Cl, -Me, —ⁱPr, —CHF₂, —CF₃, cyclopropyl, piperidin-4-yl, piperazin-4-yl, pyrazol-1-yl, pyrazol-3-yl, pyrazol-4-yl, pyrazol-5-yl, —OH, —OCH₃, —OCF₃, —OCHF₂, wherein each cyclopropyl, piperazin-4-yl, pyrazol-1-yl, pyrazol-3-yl, pyrazol-4-yl and pyrazol-5-yl, can be independently substituted with 0, 1, 2 or 3 instances of -Me, —OH, —C(═O)CH₃, —C(═O)NHCH₃, —NH₂, —NHC(═O)CH₃ or a combination thereof.

Embodiment 215. The compound of any one of embodiments 1 to 209, or a pharmaceutically acceptable salt thereof wherein each R³ is independently selected from the group consisting of —F, —Cl, -Me, —CF₃, N-Methylpiperazin-4-yl, N-methylpiperidin-4-yl, and —OCH₂CH₂N(CH₃)₂.

Embodiment 216. The compound of any one of embodiments 1 to 209, or a pharmaceutically acceptable salt thereof wherein each R³ is independently selected from the group consisting of F and —Cl.

Embodiment 217. The compound of any one of embodiments 1 to 216, or a pharmaceutically acceptable salt thereof wherein each R¹ is independently selected from the group consisting of H, halo (e.g., —F, —Cl), —CN, —C₁-C₆ alkyl (e.g., -Me, -Et, —Pr, —ⁱPr, -sec-Bu, -Bu), 5-membered heteroaryl (e.g., pyrazolyl), —C₁-C₆ haloalkyl (e.g., —CF₃, —CHF₂, —CH₂CF₃), —C₁-C₆ hydroxyalkyl (e.g., —CH₂OH), —C₃-C₉ cycloalkyl (e.g., cyclopropyl, cyclobutyl, cyclopentyl, cyclohexyl), —OR^{a1} (e.g., —OH, —OCH₃, —OCHF₂), —N(R^{a1})₂ and —C(═O)N(R^{a1})₂ (e.g., —C(═O)NH₂, —C(═O)NHCH₃).

Embodiment 218. The compound of any one of embodiments 1 to 217 wherein each R^{a1} is independently selected from the group consisting of H and —C₁-C₆ alkyl (e.g., -Me, -Et, —Pr, —ⁱPr,-ⁿBu, -^{t}Bu, -sec-Bu, -iso-Bu).

Embodiment 219. The compound of any one of embodiments 1 to 216 wherein each R¹ is independently selected from the group consisting of H and methyl.

Embodiment 220. The compound of any one of embodiments 1 to 216 wherein each R¹ is H.

Embodiment 221. The compound of any one of embodiments 1 to 216 wherein each R¹ is methyl.

Embodiment 222. The compound of any one of embodiments 1 to 221, or a pharmaceutically acceptable salt thereof, wherein n is 0 or 1.

Embodiment 223. The compound of any one of embodiments 1 to 221, or a pharmaceutically acceptable salt thereof, wherein n is 0.

Embodiment 224. The compound of any one of embodiments 1 to 221, or a pharmaceutically acceptable salt thereof, wherein n is 1.

Embodiment 225. The compound of any one of embodiments 1 to 221, or a pharmaceutically acceptable salt thereof, wherein n is 2.

Embodiment 226. The compound of any one of embodiments 1 to 221, or a pharmaceutically acceptable salt thereof, wherein n is 3.

Embodiment 227. The compound of any one of embodiments 1 to 222 and 224 to 226, or a pharmaceutically acceptable salt thereof, wherein each R² is independently selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-6 membered heterocyclyl (e.g., oxetanyl, tetrahydrofuranyl), —OR^{a2}, —N(R^{a2})₂, —C(═O)R^{a2}, —C(═O)OR^{a2}, —NR^{a4}C(═O)R^{a2}, NR^{a2}C(═O)OR^{a2}, —C(═O)N(R^{a2})₂, —OC(═O)N(R^{a2})₂.

Embodiment 228. The compound of any one of embodiments 1 to 222 and 224 to 226, or a pharmaceutically acceptable salt thereof, wherein each R² is independently selected from the group consisting of halo, —CN, —C₁-C₆ alkyl, —C₁-C₆ haloalkyl, —C₁-C₆ haloalkoxy, —C₃-C₉ cycloalkyl (e.g., cyclopropyl), 3-6 membered heterocyclyl (e.g., oxetanyl, tetrahydrofuranyl), —OR^{a2}, —N(R^{a2})₂,—C(═O)R^{a2}, —C(═O)N(OR^{a2})(R^{a2}), and —C(═O)N(R^{a2})₂.

Embodiment 229. The compound of any one of embodiments 1 to 222 and 224 to 228, or a pharmaceutically acceptable salt thereof, wherein each R^{a2} is independently selected from the group consisting of H and —C₁-C₆ alkyl (e.g., -Me, -Et, —Pr, —ⁱPr, -^{tt}Bu, -^{t}Bu, -sec-Bu, -iso-Bu).

Embodiment 230. The compound of any one of embodiments 1 to 222 and 224 to 226, or a pharmaceutically acceptable salt thereof, wherein each R² is independently selected from the group consisting of halo (e.g., —Cl), —C₁-C₆ alkyl (e.g., -Me, -Et, —Pr, —ⁱPr, -^{tt}Bu, -^{t}Bu, -sec-Bu, -iso-Bu), —C₁-C₆ haloalkyl (e.g., —CF₃, —CHF₂), —C₃-C₉ cycloalkyl (e.g., cyclopropyl), —C₁-C₆ haloalkoxy, (e.g., —OCF₃, —OCHF₂), —OCH₃, —C(═O)H, —C(═O)NHOH, and —C(═O)NH₂.

Embodiment 231. The compound of any one of embodiments 1 to 222 and 224 to 226, or a pharmaceutically acceptable salt thereof, wherein each R² is independently selected from the group consisting of halo (e.g., —Cl), —C₁-C₆ alkyl (e.g., -Me, -Et, —Pr, —ⁱPr, -^{tt}Bu, -^{t}Bu, -sec-Bu, -iso-Bu) and —OCH₃.

Embodiment 232. The compound of any one of embodiments 1 to 222 and 224 to 226, or a pharmaceutically acceptable salt thereof, wherein R² is -Me.

Embodiment 233. The compound of any one of embodiments 1 to 232 wherein the compound is selected from the group consisting of the compounds of Table 1, or a pharmaceutically acceptable salt thereof.

Embodiment 234.A pharmaceutical composition comprising a compound of any one of embodiments 1 to 233, or a pharmaceutically acceptable salt thereof, and a pharmaceutically acceptable carrier.

Embodiment 235. The pharmaceutical composition of embodiment 234, further comprising a second therapeutic agent.

Embodiment 236.A method of treating an MTAP-deficient and/or an MTA-accumulating disease in a subject in need thereof by administering to the subject an effective amount (e.g., a therapeutically effective amount) of a compound of any one of embodiments 1 to 233, or a pharmaceutically acceptable salt thereof, or a pharmaceutically acceptable composition of embodiment 234.

Embodiment 237. The method of embodiment 236 wherein the compound, or a pharmaceutically acceptable salt thereof, or composition is administered in combination with a second therapeutic agent.

Embodiment 238.A method of treating an MTAP-deficient and/or an MTA-accumulating disease in a subject in need thereof by administering to the subject an effective amount (e.g., a therapeutically effective amount) of a pharmaceutically acceptable composition of embodiment 235.

Embodiment 239. The method of any one of embodiments 236 to 238 wherein the disease is a proliferating disease.

Embodiment 240. The method of embodiment 239 wherein the disease is an MTAP-deficient and/or MTA-accumulating cancer.

Embodiment 241. The method of embodiment 240 wherein the cancer is glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 242.A method of treating a cancer in a subject in need thereof comprising the steps of:
a) assessing the level of MTAP and/or MTA in a test sample obtained from said subject, wherein the MTA level can be assessed directly (e.g., by ELISA or LC-MS/MS) or indirectly (e.g., by SDMA-modified protein ELISA or IHC, or by RNA splicing);
b) comparing the test sample with a reference, wherein MTAP deficiency and/or MTA accumulation in said test sample compared to the reference indicates the cancer in said subject will respond to therapeutic treatment with a PRMT5 inhibitor; and
c) administering a therapeutically effective amount of a compound of any one of embodiments 1 to 233, or a pharmaceutically acceptable salt thereof, or composition of embodiment 233 or 235 to the subject identified in step b).

Embodiment 243. Use of a compound of any one of embodiments 1 to 233, or a pharmaceutically acceptable salt thereof, or of a pharmaceutically acceptable composition of embodiment 234 for treating an MTAP-deficient and/or an MTA-accumulating disease in a subject in need thereof.

Embodiment 244. The use of embodiment 243 wherein the compound, or a pharmaceutically acceptable salt thereof, or composition is configured to be administered in combination with a second therapeutic agent.

Embodiment 245. Use of a pharmaceutically acceptable composition of embodiment 235 for treating an MTAP-deficient and/or an MTA-accumulating disease in a subject in need thereof.

Embodiment 246. The use of any one of embodiments 243 to 245 wherein the disease is a proliferating disease.

Embodiment 247. The use of embodiment 246 wherein the disease is an MTAP-deficient and/or MTA-accumulating cancer.

Embodiment 248. The use of embodiment 247 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 249.A compound of any one of embodiments 1 to 233, or a pharmaceutically acceptable salt thereof, or a pharmaceutically acceptable composition of embodiment 234 for treating an MTAP-deficient and/or an MTA-accumulating disease in a subject in need thereof.

Embodiment 250. The compound or composition for use of embodiment 249 wherein the compound, or a pharmaceutically acceptable salt thereof, or composition is configured to be administered in combination with a second therapeutic agent.

Embodiment 251.A pharmaceutically acceptable composition of embodiment 235 for use in treating an MTAP-deficient and/or an MTA-accumulating disease in a subject in need thereof.

Embodiment 252. The compound or composition for use of any one of embodiments 249 to 251 wherein the disease is a proliferating disease.

Embodiment 253. The compound or composition for use of embodiment 252 wherein the disease is an MTAP-deficient and/or MTA-accumulating cancer.

Embodiment 254. The compound or composition for use of embodiment 253 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

Embodiment 255. Use of a compound of any one of embodiments 1 to 233, or a pharmaceutically acceptable salt thereof, or of a pharmaceutically acceptable composition of embodiment 234 in the manufacturing of a medicament for treating an MTAP-deficient and/or an MTA-accumulating disease in a subject in need thereof.

Embodiment 256. The use of embodiment 255 wherein the medicament is configured to be administered in combination with a second therapeutic agent.

Embodiment 257. Use of a pharmaceutically acceptable composition of embodiment 235 in the manufacturing of a medicament for treating an MTAP-deficient and/or an MTA-accumulating disease in a subject in need thereof.

Embodiment 258. The use of any one of embodiments 255 to 257 wherein the disease is a proliferating disease.

Embodiment 259. The use of embodiment 258 wherein the disease is an MTAP-deficient and/or MTA-accumulating cancer.

Embodiment 260. The use of embodiment 259 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. In some embodiments, exactly one member of the group is present in, employed in, or otherwise relevant to a given product or process. In some embodiments, more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or embodiments of the invention, is/are referred to as comprising particular elements and/or features, certain embodiments of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment that falls within the prior art may be explicitly excluded from any one or more of the claims. Because such embodiments are deemed to be known to one of ordinary skill in the art, they may be excluded even if the exclusion is not set forth explicitly herein. Any particular embodiment be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

What is claimed is:

1. A compound of Formula (I) or a pharmaceutically acceptable salt thereof, wherein: 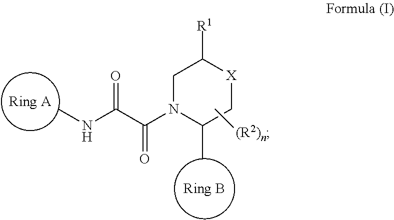
X is selected from the group consisting of O—and —NR⁷—;
Ring A is selected from the group consisting of an optionally substituted fused bicyclic 8-10 membered heteroaryl ring system containing at least one nitrogen atom, wherein the 8-10 membered refers to the total number of atoms in the fused system and optionally substituted pyridin-3-yl;
Ring B is selected from the group consisting of C₆-C₁₀ aryl and 5-10 membered heteroaryl, each optionally substituted at any available position;
each R¹ is independently absent or selected from the group consisting of H, -D, halo, —CN, —C₁-C₆ alkyl, —C₁-C₆ hydroxyalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a1}—N(R^{a1})₂, —C(═O)R^{a1}, —C(═O)OR^{a1}, —NR^{a1}C(═O)R^{a1}, —NR^{a1}C(═O)OR^{a1}, —C(═O)N(R^{a11})₂, —OC(═O)N(R^{a1})₂, —S(═O)R^{a1}, —S(═O)₂R^{a1}, —SR^{a1}, —S(═O)(═NR^{a1})R^{a1}, —NR^{a1}S(═O)₂R^{a1}and —S(═O)₂N(R^{a11})₂;
each R² is independently selected from the group consisting of -D, ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a2}, —N(R^{a2})₂, —C(═O)R^{a2}, —C(═O)OR^{a2}, —NR^{a2}C(═O)R^{a2}, —NR^{a2}C(═O)OR^{a2}, —C(═O)N(R^{a2})₂, —OC(═O)N(R^{a2})₂, —CH₂C(═O)N(R^{a2})₂, —S(═O)R^{a2}, —S(═O)₂R^{a2}, —SR^{a2}, —S(═O)(═NR^{a2})R^{a2}, —NR^{a2}S(═O)₂R^{a2} and —S(═O)₂N(R^{a2})₂ wherein two instances of R² together with the atom or atoms to which they are attached can be taken together to form a 3-10 membered cycloalkyl or heterocyclyl ring (e.g., a ring that together with the morpholine or piperazine ring of Structure I can form a bridged, fused or spiro bicyclic heterocyclic ring);
each R⁷ is independently selected from the group consisting of H, -D, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —C(═O)R^{a7}, —C(═O)OR^{a7}, —C(═O)N(R^{a7})₂, —S(═O)R^{a7}, —S(═O)₂R^{a7} and —S(=O)₂N(R^{a7})₂, wherein each alkyl, cycloalkyl, heterocyclyl, aryl, heteroaryl, arylalkyl and heteroarylalkyl is optionally substituted at any available position;
each R^{a1}, R^{a2} and R^{a7} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R^{b}, —C(═O)OR^{b}, —NR^{b}C(═O)R, —NR^{b}C(═O)OR^{b}, —C(═O)N(R^{b})₂, —OC(═O)N(R^{b})₂, —S(═O)R^{cc}, —S(═O)₂R^{b}, —SR^{b}, —S(═O)(═NR)R, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl); and
n is 0, 1, 2 or 3;

2. The compound of claim 1, or a pharmaceutically acceptable salt thereof, wherein R¹ is not absent or H and Ring B and R¹ are in a trans relative configuration.

3. The compound of claim 1, or a pharmaceutically acceptable salt thereof, wherein R¹ is not absent or H and Ring B and R¹ are in a cis relative configuration.

4. The compound of claim 1 or 2, or a pharmaceutically acceptable salt thereof, wherein the moiety represented as 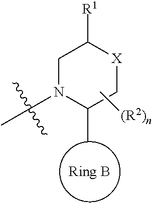
is selected from the group consisting of:

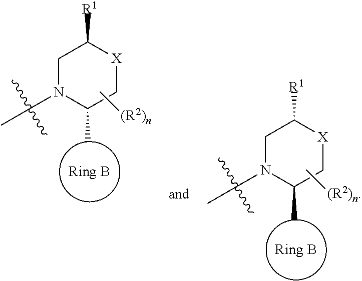

5. The compound of claim 4, or a pharmaceutically acceptable salt thereof, wherein the compound is of Formula (Ia) or Formula (Ib):

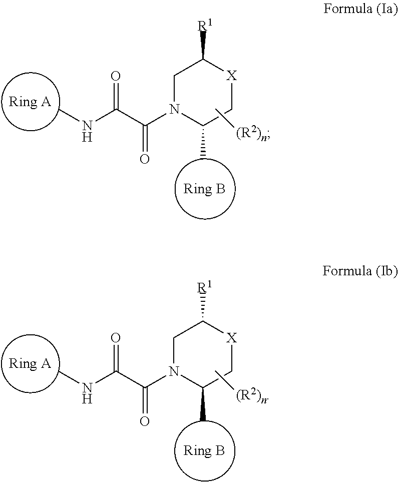

6. The compound of claim 4, or a pharmaceutically acceptable salt thereof, wherein the compound is of Formula (Ia).

7. The compound of claim 4, or a pharmaceutically acceptable salt thereof, wherein the compound is of Formula (Ib).

8. The compound of any one of claims 1-7 or a pharmaceutically acceptable salt thereof, wherein X is —O—.

9. The compound of claim 8 or a pharmaceutically acceptable salt thereof, wherein the compound is of formula (II):

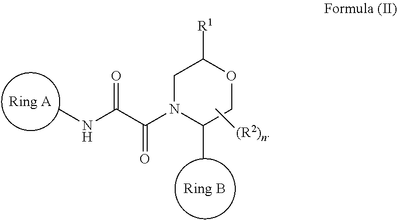

10. The compound of any one of claims 1-7 or a pharmaceutically acceptable salt thereof, wherein X is —NR⁷—.

11. The compound of claim 10 or a pharmaceutically acceptable salt thereof, wherein the compound is of formula (III):

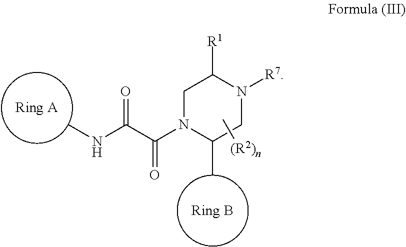

12. The compound of claim 11, or a pharmaceutically acceptable salt thereof, wherein the compound is of Formula (IIIa), Formula (IIIb), Formula (IIIc) or Formula (IIId):

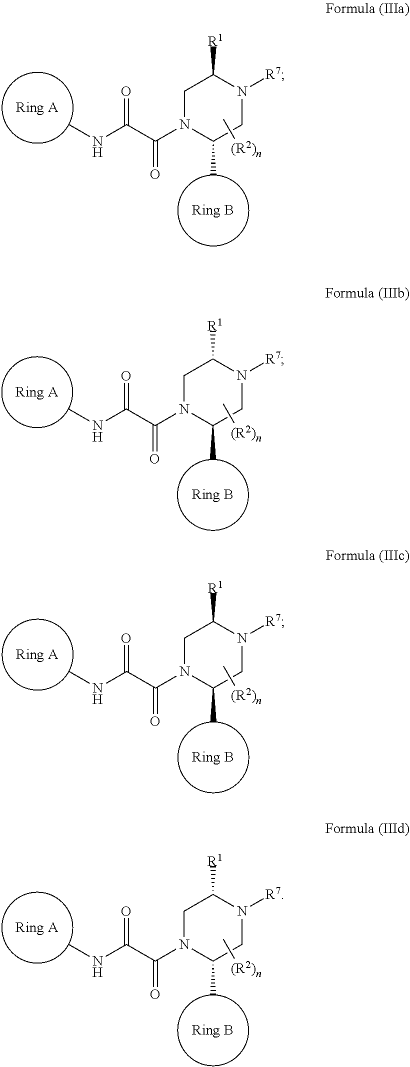

13. The compound of claim 12, or a pharmaceutically acceptable salt thereof, wherein the compound is of Formula (IIIa) or Formula (IIIb).

14. The compound of any one of claims 1 to 13, or a pharmaceutically acceptable salt thereof, wherein Ring A is selected from the group consisting of an optionally substituted fused bicyclic 8-10 membered heteroaryl ring system containing at least one nitrogen atom, wherein the 8-10 membered refers to the total number of atoms in the fused system and pyridin-3-yl, each substituted at any available positions with 0, 1, 2, 3 or 4 instances of R⁴, wherein:
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, NR^{a4}S(═O)₂R^{a4} and —S(═O)₂N(R^{a4})₂; and
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R, —C(=)OR^{b}, —NR^{b}C(═O)R, —NR^{b}C(═O)OR^{cc}, —C(═O)N(R^{b})₂, —OC(═O)N(R^{b})₂, —S(═O)R^{b}, —S(═O)₂R, —SR^{b}, —S(═O)(═NR)R^{cc}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -Bu, -sec-Bu, -iso-Bu)), and C₃-C₉ cycloalkyl (e.g., cyclopropyl, cyclobutyl, cyclopentyl, cyclohexyl).

15. The compound of any one of claims 1 to 14, or a pharmaceutically acceptable salt thereof, wherein Ring A is selected from the group consisting of: 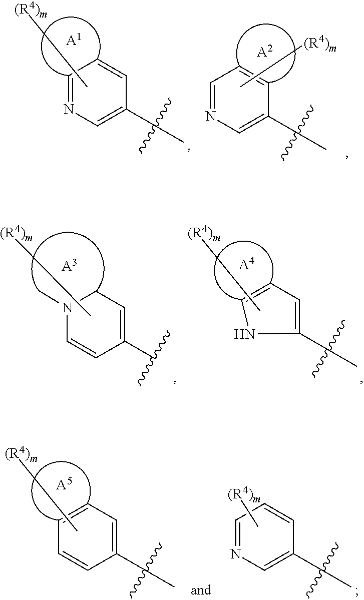
wherein
each of rings A¹, A² and A⁴ is independently 4-6 membered carbocyclyl, 4-6 membered heterocyclyl, 5-6 membered heteroaryl or phenyl;
each ring A³ is independently a 4-6 membered heterocyclyl or 5-6 membered heteroaryl, wherein the heterocyclyl and heteroaryl contain at least one nitrogen atom
each ring A⁵ is independently a 5-6 membered heteroaryl, wherein the heteroaryl contains at least one nitrogen atom;
each R⁴ is independently selected from the group consisting of halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, NR^{a4}S(═O)₂R^{a4} and —S(═O)₂N(R^{a4})₂;
each R^{a4} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{b}, —N(R^{b})₂, —C(═O)R^{b}, —C(═O)OR, —NR^{b}C(═O)R, —NR^{b}C(═O)OR, —C(═O)N(R^{b})₂, —OC(═O)N(R^{b})₂, —S(═O)R, —S(═O)₂R, —SR, —S(═O)(═NR^{b})R^{b}, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -Bu, -sec-Bu, -iso-Bu)), and C₃-C₉ cycloalkyl (e.g., cyclopropyl, cyclobutyl, cyclopentyl, cyclohexyl); and
m is 0, 1, 2, 3 or 4.

16. The compound of claim 15, or a pharmaceutically acceptable salt thereof, wherein Ring A is selected from the group consisting of:

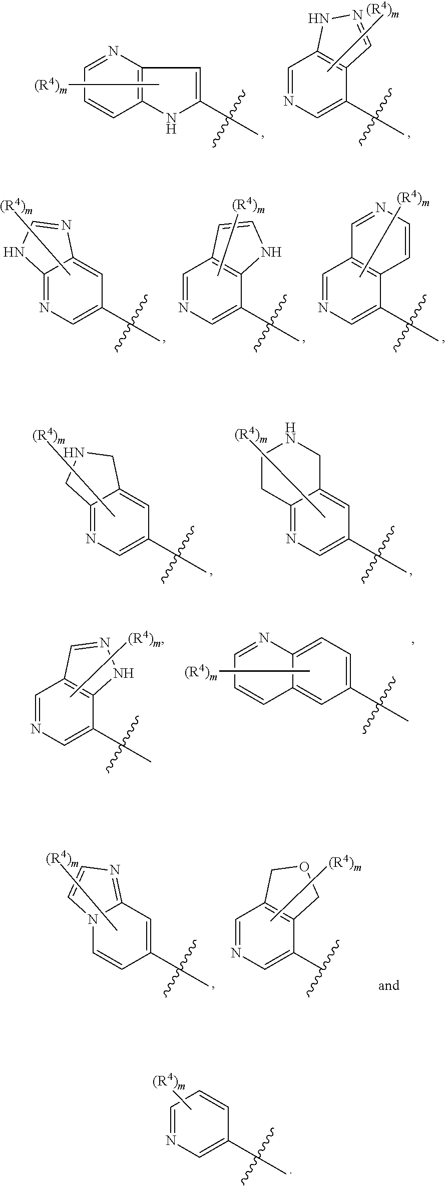

17. The compound of claim 15, or a pharmaceutically acceptable salt thereof, wherein Ring A is selected from the group consisting of:

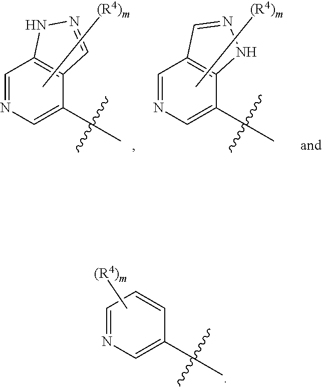

18. The compound of claim 15, or a pharmaceutically acceptable salt thereof, wherein Ring A is:

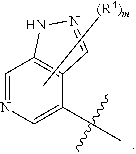

19. The compound of claim 15, or a pharmaceutically acceptable salt thereof, wherein Ring A is:

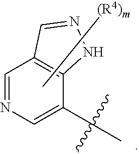

20. The compound of claim 15, or a pharmaceutically acceptable salt thereof, wherein Ring A is:

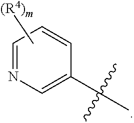

21. The compound of claim 16, or a pharmaceutically acceptable salt thereof, wherein Ring A is selected from the group consisting of: 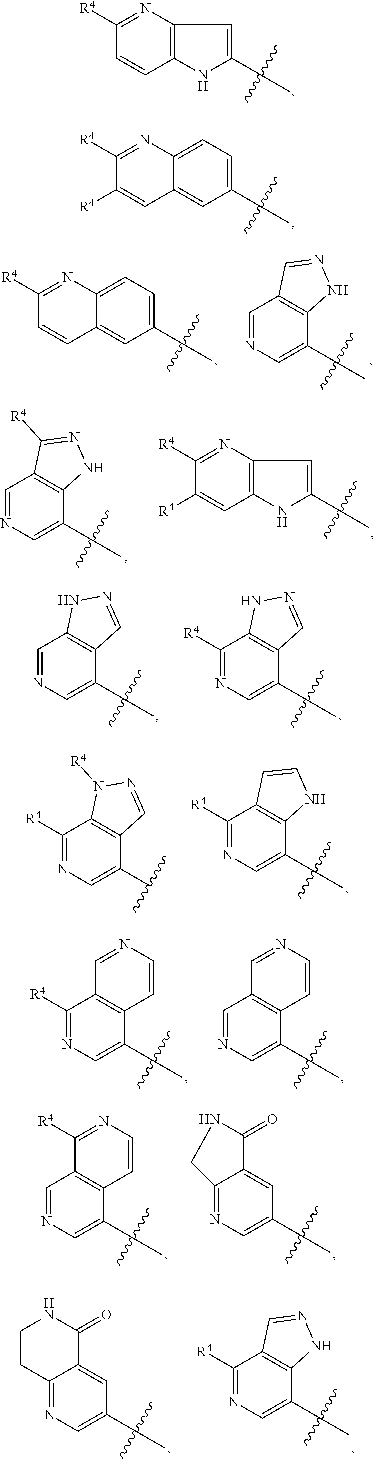 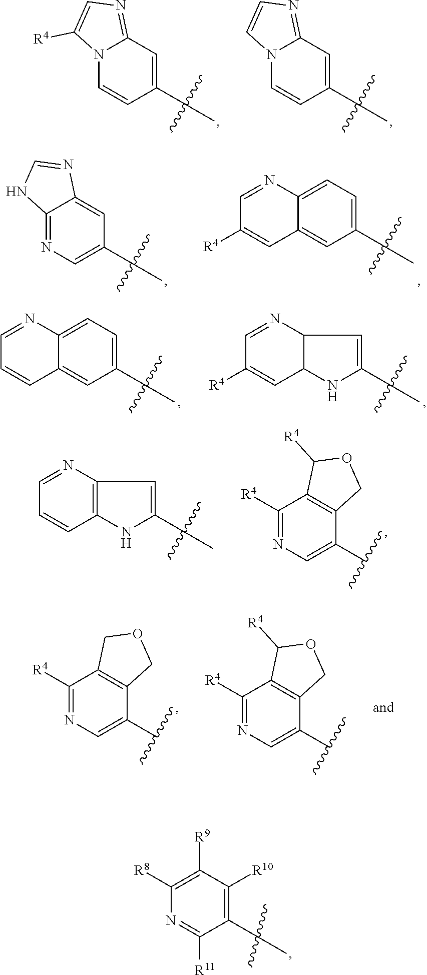
wherein
each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a4}, —N(R^{a4})₂, —C(═O)R^{a4}, —C(═O)OR^{a4}, —NR^{a4}C(═O)R^{a4}, —NR^{a4}C(═O)OR^{a4}, —C(═O)N(R^{a4})₂, —C(═O)N(OR^{a4})(R^{a4}), —OC(═O)N(R^{a4})₂, —S(═O)R^{a4}, —S(═O)₂R^{a4}, —SR^{a4}, —S(═O)(═NR^{a4})R^{a4}, —NR^{a4}S(═O)₂R^{a4} and —S(═O)₂N(R^{a4})₂;
each R⁸ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a8}, —N(R^{a8})₂, —C(═O)R^{a8}, —C(═O)OR^{a8}, —NR^{a8}C(═O)R^{a8}, —NR^{a8}C(═O)OR^{a8}, —C(═O)N(R^{a8})₂, —OC(═O)N(R^{a8})₂, —S(═O)R^{a8}, —S(═O)₂R^{a8}, —SR^{a8}, —S(═O)(═NR^{a8})R^{a8}, —NR^{a8}S(═O)₂R^{a8} and —S(═O)₂N(R^{a8})₂;
each R⁹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a9}, —N(R^{a9})₂, —C(═O)R^{a9}, —C(═O)OR^{a9}, —NR^{a9}C(═O)R^{a9}, —NR^{a9}C(═O)OR^{a9}, —C(═O)N(R^{a9})₂, —OC(═O)N(R^{a9})₂, —S(═O)R^{a9}, —S(═O)₂R^{a9}, —SR^{a9}, —S(═O)(═NR^{a9})R^{a9}, —NR^{a9}S(═O)₂R^{a9} and —S(=O)₂N(R^{a9})₂;
each R¹⁰ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a10}, —N(R^{a10})₂—C(═O)R^{a10}, —C(═O)OR^{a10}, —NR^{a1}OC(═O)R^{a10}, —NR^{a1}OC(═O)OR^{a10}, —C(═O)N(R^{a10})₂—OC(═O)N(R^{a10})₂, —S(═O)R^{a10}, —S(═O)₂R^{a10}, —SR^{a1}O, —S(═O)(═NR^{a1}O)R^{a1}O, —NR^{a10}S(═O)₂R^{a10} and —S(═O)₂N(R^{a10})₂;
each R¹¹ is independently selected from the group consisting of H, -D, halo, —CN, —C₁-C₆ alkyl, —C₁-C₆ heteroalkyl, —C₁-C₆ haloalkyl, —C₃-C₉ cycloalkyl, 3-10 membered heterocyclyl, heterocyclylalkyl, heteroarylalkyl, arylalkyl, cycloalkylalkyl, —OR^{a11}, —N(R^{a1})₂, —C(═O)R^{a11}, —C(═O)OR^{a11}, —NR^{a11}C(═O)R^{a11}, —NR^{a11}C(═O)OR^{a1}, —C(═O)N(Ra)₂, —OC(═O)N(Ran)₂, —S(═O)Ran, —S(═O)₂Ran, —SR^{a11}—S(═O)(═NR^{a11})Ran, —NR^{a11}S(═O)₂Ran and —S(═O)₂N(Ra)₂;
each R^{a4}, R^{a8}, R^{a9}, R^{a10} and R^{a1} is independently selected from the group consisting of H, —C₁-C₆ alkyl, —C₁-C₆ heteroalkyl, —C₁-C₆ haloalkyl, C₃-C₉ cycloalkyl, 3-10 membered heterocyclyl, cycloalkylalkyl, heterocyclylalkyl, C₆-C₁₀ aryl, 5-10 membered heteroaryl, arylalkyl and heteroarylalkyl wherein each alkyl, cycloalkyl, heterocyclyl, cycloalkylalkyl, heterocyclylalkyl, aryl, heteroaryl, arylalkyl and heteroarylalkyl is optionally substituted at any available position (e.g., substituted with 0, 1, 2 or 3 instances of R⁵, wherein each R⁵ is independently selected from the group consisting of ═O, halo, —CN, —C₁-C₆ alkyl, —C₁-C₆ heteroalkyl, —C₁-C₆ hydroxyalkyl,—C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR, —N(R^{b})₂, —C(═O)R, —C(═O)OR, —NR^{b}C(═O)R, —NR^{b}C(═O)OR, —C(═O)N(R^{b})₂, —OC(═O)N(R^{b})₂, —S(═O)R^{cc}, —S(═O)₂R^{b}, —SR^{b}, —S(═O)(═NR^{b})R, —NR^{b}S(═O)₂R^{b} and —S(═O)₂N(R^{b})₂, wherein each R^{b} is independently selected from the group consisting of H, —C₁-C₆ alkyl (e.g., -Me, -Et, —Pr, —ⁱPr, -^{tt}Bu, -^{t}Bu, -sec-Bu, -iso-Bu).and C₃-C₉ cycloalkyl (e.g., cyclopropyl, cyclobutyl, cyclopentyl, cyclohexyl).

22. The compound of claim 21, or a pharmaceutically acceptable salt thereof, wherein Ring A is selected from the group consisting of:

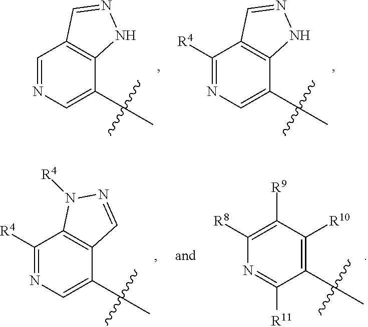

23. The compound of claim 21, or a pharmaceutically acceptable salt thereof, wherein Ring A is selected from the group consisting of:

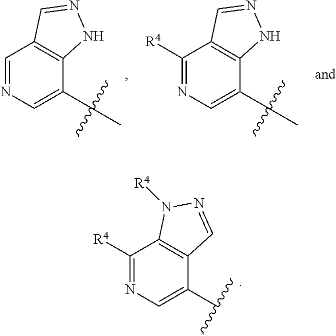

24. The compound of any one of claims 14 to 23, or a pharmaceutically acceptable salt thereof, wherein each R⁴ is independently selected from the group consisting of -D, halo, ═O, —CN, —C₁-C₆ alkyl, —C₁-C₆ heteroalkyl, —C₁-C₆ haloalkyl, —C₃-C₉ cycloalkyl, —OR^{a4}, —N(R^{a4})₂, —C(═O)R^{a4}, —C(=)OR^{a4}, —NR^{a4}C(═O)R^{a4}, —NR^{a4}C(═O)OR^{a4}, —C(═O)N(R^{a4})₂, —C(═O)N(OR^{a4})(R^{a4}) and —OC(═O)N(R^{a4})₂.

25. The compound of any one of claims 14 to 23, or a pharmaceutically acceptable salt thereof, wherein each R⁴ is independently selected from the group consisting of ═O, —C₁-C₆ alkyl and —N(R^{a4})₂.

26. The compound of any one of claims 14 to 25, or a pharmaceutically acceptable salt thereof, wherein each R^{a4} is H.

27. The compound of any one of claims 14 to 23, or a pharmaceutically acceptable salt thereof, wherein each R⁴ is independently —NH₂ or -Me.

28. The compound of claim 21 or 22, or a pharmaceutically acceptable salt thereof, wherein Ring A is

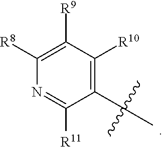

29. The compound of any one of claims 21, 22 and 28, or a pharmaceutically acceptable salt thereof, wherein R¹⁰ is selected from the group consisting of H, halo, —CN, —C₁-C₆ alkyl, —C₁-C₆ haloalkyl and —N(R^{a10})₂.

30. The compound of any one of claims 21, 22, and 28-29, or a pharmaceutically acceptable salt thereof, wherein R^{a10} is selected from the group consisting of H and —C₁-C₆ alkyl (e.g., -Me, -Et, —Pr, -iPr, -nBu, -tBu, -sec-Bu, -iso-Bu).

31. The compound of any one of claims 21, 22 and 28, or a pharmaceutically acceptable salt thereof, wherein R¹⁰ is selected from the group consisting of H and -Me.

32. The compound of any one of claims 21, 22 and 28, or a pharmaceutically acceptable salt thereof, wherein R¹⁰ is —H.

33. The compound of any one of claims 21, 22 and 28-32, or a pharmaceutically acceptable salt thereof, wherein R¹¹ is selected from the group consisting of H, halo, —CN, —C₁-C₆ alkyl, —C₁-C₆ haloalkyl and —N(Ra)₂.

34. The compound of claim 21, 22 and 28-33, or a pharmaceutically acceptable salt thereof, wherein each R^{a11} is independently selected from the group consisting of H and —C₁-C₆ alkyl (e.g., -Me, -Et, —Pr, -iPr, -nBu, -tBu, -sec-Bu, -iso-Bu).

35. The compound of any one of claims 21, 22 and 28-32, or a pharmaceutically acceptable salt thereof, wherein R¹¹ is H.

36. The compound of any one of claims 21, 22 and 28, or a pharmaceutically acceptable salt thereof, wherein ring A is

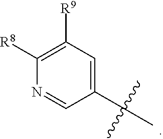

37. The compound of any one of claims 21, 22 and 28-36, or a pharmaceutically acceptable salt thereof, wherein R⁸ is selected from the group consisting of H, —C₁-C₆ alkyl, —C₁-C₆ haloalkyl, —OR^{as} and —N(R^{a8})₂.

38. The compound of any one of claims 21, 22 and 28-36, or a pharmaceutically acceptable salt thereof, wherein R⁸ is selected from the group consisting of —OR^{as} and —N(R^{a8})₂.

39. The compound of any one of claims 21, 22 and 28-38, or a pharmaceutically acceptable salt thereof, wherein each R^{as} is independently selected from the group consisting of H, —C₁-C₆ alkyl (e.g., -Me, -Et, —Pr, —ⁱPr, -nBu, -^{t}Bu, -sec-Bu, -iso-Bu) and —C₁-C₆ haloalkyl (e.g., —CHF₂, —CF₃).

40. The compound of any one of claims 21, 22 and 28-36, or a pharmaceutically acceptable salt thereof, wherein R⁸ is selected from the group consisting of H, -Me, —CHF₂, —OCH₃ and —NH₂.

41. The compound of any one of claims 21, 22 and 28-36, or a pharmaceutically acceptable salt thereof, wherein R⁸ is selected from the group consisting of NH₂ and —OCH₃.

42. The compound of any one of claims 21, 22 and 28-36, or a pharmaceutically acceptable salt thereof, wherein R⁸ is —OCH₃.

43. The compound of any one of claims 21, 22 and 28-36, or a pharmaceutically acceptable salt thereof, wherein R⁸ is —NH₂.

44. The compound of any one of claims 21, 22 and 28-43, or a pharmaceutically acceptable salt thereof, wherein R⁹ is selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-10 membered heterocyclyl, —OR^{a9}, —N(R^{a9})₂, —C(═O)R^{a9}, —C(═O)OR^{a9}, —NR^{a9}C(═O)R^{a9}, —NR^{a9}C(═O)OR^{a9}, —C(═O)N(R^{a9})₂, —OC(═O)N(R^{a9})₂.

45. The compound of any one of claims 21, 22 and 28-43, or a pharmaceutically acceptable salt thereof, wherein R⁹ is selected from the group consisting of —C₁-C₆ alkyl, 3-10 membered heterocyclyl (e.g., oxetanyl), —C₃-C₉ cycloalkyl (e.g., cyclopropyl) and —C(═O)N(R^{a9})₂.

46. The compound of any one of claims 21, 22 and 28-45, or a pharmaceutically acceptable salt thereof, wherein each R^{a9} is independently selected from the group consisting of H and —C₁-C₆ alkyl (e.g., -Me, -Et, —Pr, -iPr, -nBu, -tBu, -sec-Bu, -iso-Bu).

47. The compound of any one of claims 21, 22 and 28-43, or a pharmaceutically acceptable salt thereof, wherein R⁹ is selected from the group consisting of —C₁-C₆ alkyl (e.g., -Me, -Et, —Pr, -iPr, -nBu, -tBu, -sec-Bu, -iso-Bu), 3-10 membered heterocyclyl (e.g., oxetan-3-yl), —C₃-C₉ cycloalkyl (e.g., cyclopropyl) and —C(═O)NH₂.

48. The compound of any one of claims 21, 22 and 28-43, or a pharmaceutically acceptable salt thereof, wherein R⁹ is selected from the group consisting of -Me, -Et, oxetan-3-yl, cyclopropyl and —C(═O)NH₂.

49. The compound of any one of claims 21, 22 and 28-43, or a pharmaceutically acceptable salt thereof, wherein R⁹ is —C(═O)NH₂.

50. The compound of any one of claims 21, 22 and 28-43, or a pharmaceutically acceptable salt thereof, wherein R⁹ is selected from the group consisting of -Me, -Et, oxetan-3-yl and cyclopropyl.

51. The compound of any one of claims 21, 22 and 28-36, or a pharmaceutically acceptable salt thereof, wherein R⁸ is —OCH₃ and R⁹ is —C(═O)NH₂.

52. The compound of any one of claims 21, 22 and 28-36, or a pharmaceutically acceptable salt thereof, wherein R⁸ is —NH₂ and R⁹ is selected from the group consisting of -Me, -Et, oxetan-3-yl, cyclopropyl.

53. The compound of claim 21, or a pharmaceutically acceptable salt thereof, wherein Ring A is selected from the group consisting of:

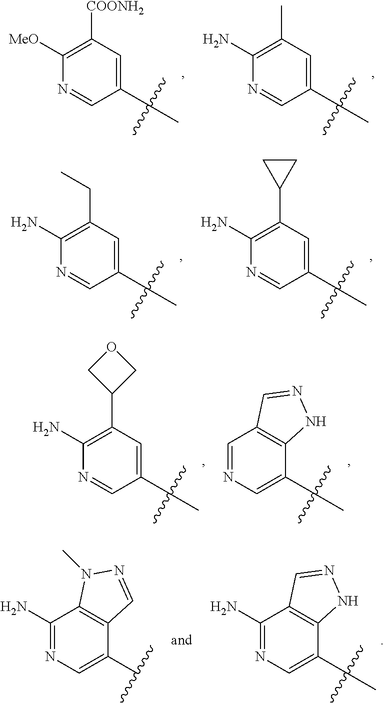

54. The compound of claim 21, or a pharmaceutically acceptable salt thereof, wherein Ring A is selected from the group consisting of:

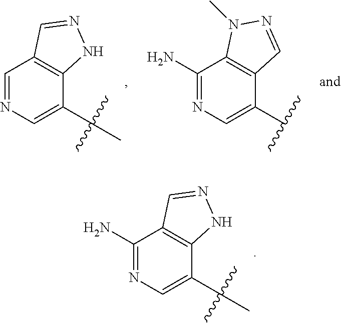 [SMILES: *c1cncc2cn[nH]c12 ; *c1cnc(N)c2c1cnn2C ; *c1cnc(N)c2cn[nH]c12]

55. The compound of claim 21, or a pharmaceutically acceptable salt thereof, wherein Ring A is selected from the group consisting of:

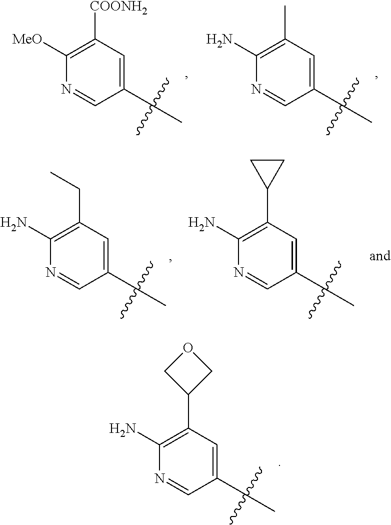

56. The compound of any one of claims 1 to 7 and 10 to 55, or a pharmaceutically acceptable salt thereof, wherein each R⁷ is independently selected from the group consisting of —C₁-C₆ alkyl (e.g., -Me, -Et, —Pr, —ⁱPr, -^{tt}Bu, -^{t}Bu, -sec-Bu, -iso-Bu, neopentyl), —C₃-C₉ cycloalkyl (e.g., cyclopropyl, cyclobutyl, cyclopentyl, cyclohexyl), —C(═O)R^{a7} and —C(═O)OR^{a7}, wherein the alkyl and cycloalkyl is substituted at any available position with 0, 1, 2 or 3 instances of —OH, —OCH₃, —CN, halo (e.g., —Cl, —F), —NH₂, —C₁-C₆ alkyl (e.g., -Me, -Et), —C₁-C₆ haloalkyl (e.g., —CF₃, —CHF₂).

57. The compound of any one of claims 1 to 7 and 10 to 55, or a pharmaceutically acceptable salt thereof, wherein each R⁷ is independently selected from the group consisting of -Me, -Et, —Pr, —ⁱPr, -^{tt}Bu, -^{t}Bu, -sec-Bu, -iso-Bu, neopentyl, cyclopropyl, cyclobutyl, —C(═O)R^{a7} and —C(═O)OR^{a7}, wherein the cyclopropyl and cyclobutyl is substituted at any available position with 0, 1 or 2 instances of -Me.

58. The compound of any one of claims 1 to 7 and 10 to 55, or a pharmaceutically acceptable salt thereof, wherein each R⁷ is —C(═O)R^{a7}.

59. The compound of any one of claims 1 to 7 and 10 to 55, or a pharmaceutically acceptable salt thereof, wherein each R⁷ is —C(═O)OR^{a7}.

60. The compound of any one of claims 1 to 7 and 10 to 59, or a pharmaceutically acceptable salt thereof, wherein each R^{a7} is independently selected from the group consisting of H, —C₁-C₆ alkyl (e.g., -Me, -Et, —Pr, —ⁱPr, -^{tt}Bu, -^{t}Bu, -sec-Bu, -iso-Bu), C₃-C₉ cycloalkyl (e.g., cyclopropyl, cyclobutyl, cyclopentyl, cyclohexyl, cycloheptyl, bicyclo [1.1.1]pentyl, spiro[2.2]pentyl, bicyclo[2.1.0]pentyl, bicyclo[3.1.0]hexyl), cycloalkylalkyl (e.g., —CH₂-cyclopropyl, —CH₂-cyclobutyl, —CH₂-cyclopentyl, —CH₂-cyclohexyl, —CH₂-cycloheptyl), wherein each alkyl, cycloalkyl, and cycloalkylalkyl is substituted at any available position with 0, 1, 2 or 3 instances of R⁵, wherein each R⁵ is independently selected from the group consisting of ═O, halo (e.g., —F, —Cl), —CN, —C₁-C₆ alkyl (e.g., -Me, -Et, iPr), —C₁-C₆ heteroalkyl, —C₁-C₆ hydroxyalkyl (e.g., —CH₂OH), —C₁-C₆ haloalkyl (e.g., —CF₃, —CH₂CF₃, —CF₂CH₃, —CHF₂, —CH₂F), 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), —OH and —OC₁-C₆ alkyl (e.g., —OCH₃).

61. The compound of any one of claims 1 to 7 and 10 to 59, or a pharmaceutically acceptable salt thereof, wherein each R^{a7} is independently —C₁-C₆ alkyl (e.g., -Me, -Et, —Pr, —ⁱPr, -^{tt}Bu, -^{t}Bu, -sec-Bu, -iso-Bu) substituted with 0, 1, 2 or 3 instances of R⁵, wherein each R is independently selected from the group consisting of halo (e.g., —F, —Cl), —CN, —C₁-C₆ haloalkyl (e.g., —CF₃, —CH₂CF₃, —CF₂CH₃, —CHF₂, —CH₂F) 3-10 membered heterocyclyl (e.g., N-Me-piperazinyl, 1,2,3,4-tetrahydropyrrolo[1,2-a]pyrazinyl), —N(R^{b})₂ (e.g., —N(CH₃)₂), and —OH.

62. The compound of any one of claims 1 to 7 and 10 to 61, or a pharmaceutically acceptable salt thereof, wherein each R⁵ is independently selected from the group consisting of -Me, —CF₃, —N(CH₃)₂, N-Me-piperazinyl and 1,2,3,4-tetrahydropyrrolo[1,2-a]pyrazin-2-yl.

63. The compound of any one of claims 1 to 7 and 10 to 59, or a pharmaceutically acceptable salt thereof, wherein each R^{a7} is independently selected from the group consisting of -Me, -Et, —ⁱPr, -Bu, -iso-Bu, cyclopropyl, —CH₂-cyclopropyl, each substituted at any available position with 0, 1, 2 or 3 instances of R⁵, wherein each R is independently selected from the group consisting of Me, —CF₃, —N(CH₃)₂, N-Me-piperazinyl and 1,2,3,4-tetrahydropyrrolo[1,2-a]pyrazin-2-yl.

64. The compound of any one of claims 1 to 7 and 10 to 55, or a pharmaceutically acceptable salt thereof, wherein each R⁷ is independently C₁-C₆ alkyl (e.g., -Me, -Et, —Pr, —ⁱPr, -^{tt}Bu, -^{t}Bu, -sec-Bu, -iso-Bu, neopentyl).

65. The compound of any one of claims 1 to 7 and 10 to 55, or a pharmaceutically acceptable salt thereof, wherein each R⁷ is independently selected from the group consisting of -Me, -Et, —ⁱPr, -iso-Bu, neopentyl.

66. The compound of any one of claims 1 to 7 and 10 to 55, or a pharmaceutically acceptable salt thereof, wherein each R⁷ is independently selected from the group consisting of:

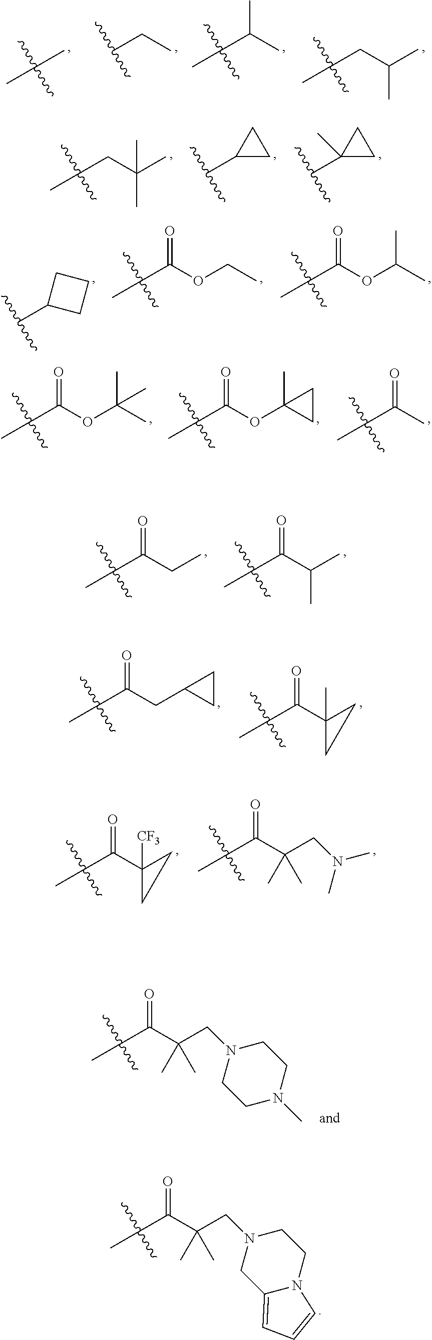

67. The compound of any one of claims 1 to 68, or a pharmaceutically acceptable salt thereof, wherein Ring B is selected from the group consisting of C₆-C₁₀ aryl and 8-10 membered bicyclic heteroaryl wherein the aryl and heteroaryl are optionally substituted at any available position.

68. The compound of any one of claims 1 to 68, or a pharmaceutically acceptable salt thereof, wherein Ring B is selected from the group consisting of thiophenyl, phenyl and benzo[d]thiazolyl, each optionally substituted.

69. The compound of any one of claims 1 to 68, or a pharmaceutically acceptable salt thereof, wherein Ring B is phenyl, optionally substituted at any available position.

70. The compound of any one of claims 1 to 68, or a pharmaceutically acceptable salt thereof, wherein Ring B is 5-6 membered monocyclic heteroaryl wherein the heteroaryl is optionally substituted at any available position.

71. The compound of any one of claims 1 to 68, or a pharmaceutically acceptable salt thereof, wherein Ring B is thiophenyl, optionally substituted.

72. The compound of any one of claims 1 to 68, or a pharmaceutically acceptable salt thereof, wherein Ring B is an 8-10 membered bicyclic heteroaryl, wherein the bicyclic heteroaryl is optionally substituted at any available position.

73. The compound of any one of claims 1 to 68, or a pharmaceutically acceptable salt thereof, wherein Ring B is benzo[d]thiazolyl, optionally substituted.

74. The compound of any one of claims 1 to 73 wherein each Ring B is substituted at any available position with 0, 1, 2 or 3 instances of R³, wherein:
each R³ is independently selected from the group consisting of -D, ═O, —CN, halo, —C₁-C₆ alkyl, —C₁-C₆ heteroalkyl, —C₁-C₆ haloalkyl, —C₃-C₉ cycloalkyl, 3-10 membered heterocyclyl, C₆-C₁₀ aryl, 5-10 membered heteroaryl, cycloalkylalkyl, heterocyclylalkyl, arylalkyl, heteroarylalkyl, —OR^{a3}, —N(R^{a3})₂, —C(═O)R^{a3}, —C(═O)OR^{a3}, —NR^{a3}C(═O)R^{a3}, —NR^{a3}C(═O)OR^{a3}, —C(═O)N(R^{a3})₂, —OC(═O)R^{a3}, —OC(═O)N(R^{a3})₂, —S(═O)R^{a3}, —S(═O)₂R^{a3}, —SR^{a3}, —S(═O)(═NR^{a3})R^{a3}, —NR^{a3}S(═O)₂R^{a3} and —S(═O)₂N(R^{a3})₂, wherein each alkyl, cycloalkyl, heterocyclyl, aryl, heteroaryl, cycloalkylalkyl, heterocyclylalkyl, arylalkyl and heteroarylalkyl of R³ is optionally substituted (e.g., substituted with 0, 1, 2 or 3 instances of -Me, —OH, —C(═O)CH₃, —C(═O)NHCH₃, —NH₂, —NHC(═O)CH₃ or a combination thereof);
each R^{a3} is independently H; —C₁-C₆ alkyl; —C₁-C₆ haloalkyl; —C₁-C₆ heteroalkyl substituted with 0 or 1 instance of ═O; C₃-C₉ cycloalkyl; or 3-10 membered heterocyclyl substituted with 0 or 1 instances of ═O, -Me or a combination thereof.

75. The compound of claim 74, or a pharmaceutically acceptable salt thereof, wherein Ring B is selected from the group consisting of:

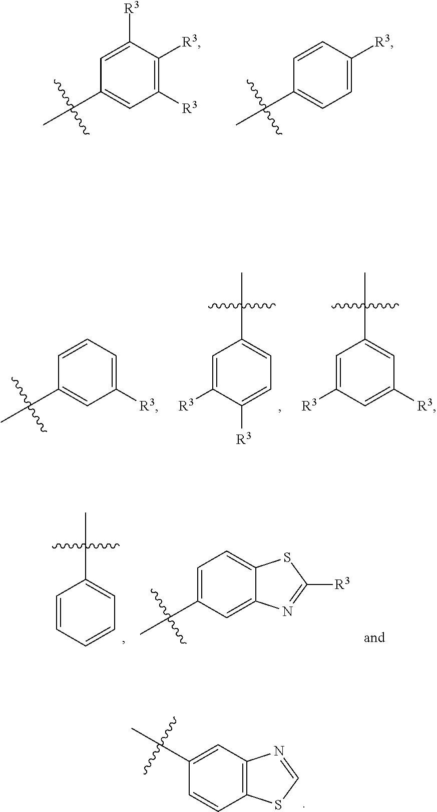

76. The compound of claim 74 or 75, or a pharmaceutically acceptable salt thereof wherein each R³ is independently selected from the group consisting of —F, —Cl, -Me, —CF₃, N-Methylpiperazin-4-yl, N-methylpiperidin-4-yl, and —OCH₂CH₂N(CH₃)₂.

77. The compound of claim 74 or 75, or a pharmaceutically acceptable salt thereof wherein each R³ is independently selected from the group consisting of —F and —Cl.

78. The compound of any one of claims 1 to 77 wherein each R¹ is independently selected from the group consisting of H and methyl.

79. The compound of any one of claims 1 to 77 wherein each R¹ is H.

80. The compound of any one of claims 1 to 77 wherein each R¹ is methyl.

81. The compound of any one of claims 1 to 80, or a pharmaceutically acceptable salt thereof, wherein n is 0 or 1.

82. The compound of any one of claims 1 to 81, or a pharmaceutically acceptable salt thereof, wherein each R² is independently selected from the group consisting of halo, —CN, —C₁-C₆ alkyl, —C₁-C₆ heteroalkyl, —C₁-C₆ haloalkyl, —C₃-C₉ cycloalkyl (e.g., cyclopropyl), 3-6 membered heterocyclyl (e.g., oxetanyl, tetrahydrofuranyl), —OR^{a2}, —N(R^{a2})₂, —C(═O)R^{a2}—C(═O)OR^{a2}, —NR^{a4}C(═O)R^{a2}, —NR^{a2}C(═O)OR^{a2}, —C(═O)N(R^{a2})₂, —OC(═O)N(R^{a2})₂.

83. The compound of any one of claims 1 to 81, or a pharmaceutically acceptable salt thereof, wherein each R^{a2} is independently selected from the group consisting of H and —C₁-C₆ alkyl (e.g., -Me, -Et, —Pr, —ⁱPr, -^{tt}Bu, -^{t}Bu, -sec-Bu, -iso-Bu).

84. The compound of any one of claims 1 to 81, or a pharmaceutically acceptable salt thereof, wherein each R² is independently selected from the group consisting of halo (e.g., —C₁), —C₁-C₆ alkyl (e.g., -Me, -Et, —Pr, —ⁱPr, -^{tt}Bu, -Bu, -sec-Bu, -iso-Bu) and —OCH₃.

85. The compound of any one of claims 1 to 81, or a pharmaceutically acceptable salt thereof, wherein R² is -Me.

86. The compound of any one of claims 1 to 85, wherein the compound is selected from the group consisting of: 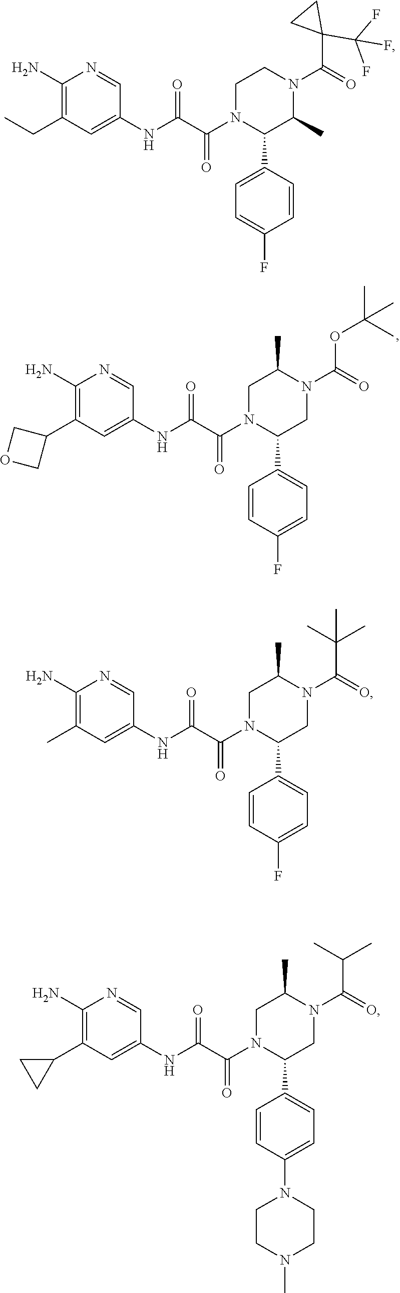 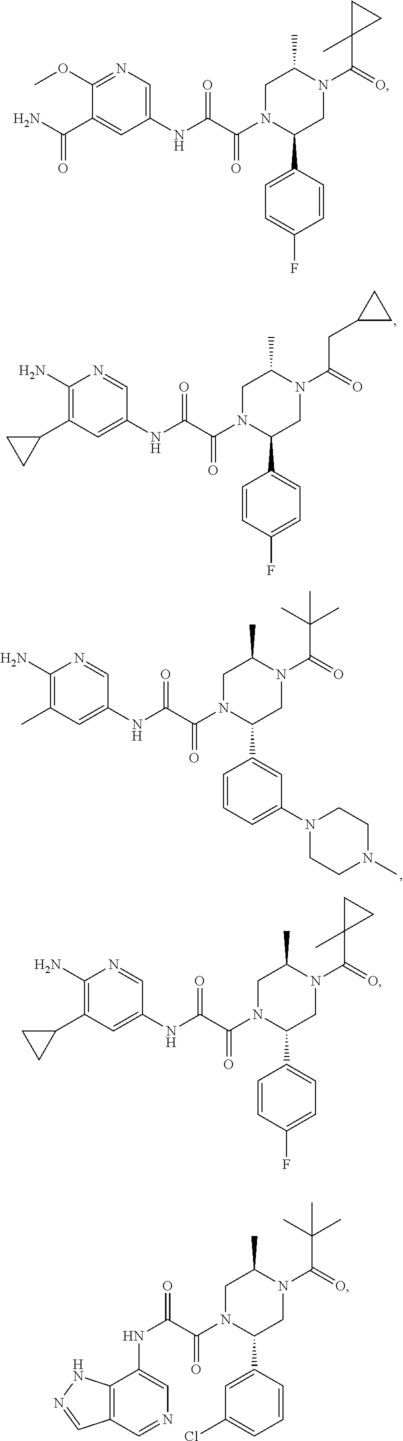 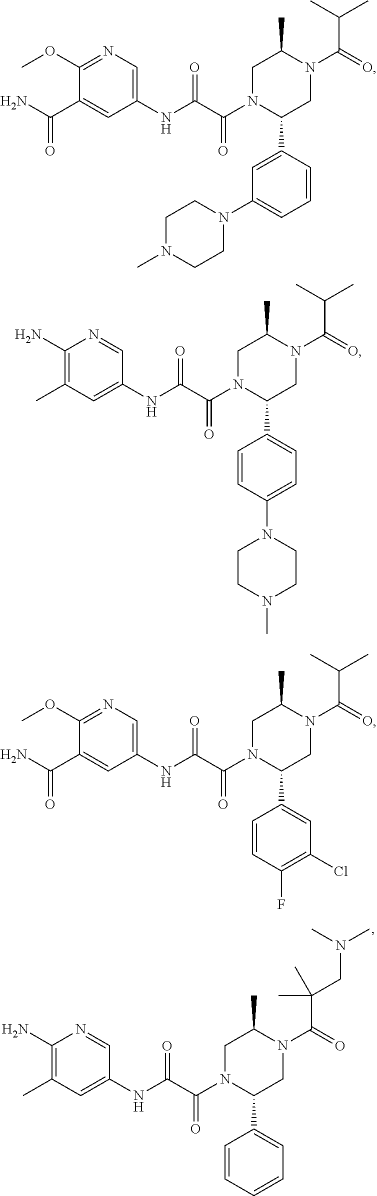 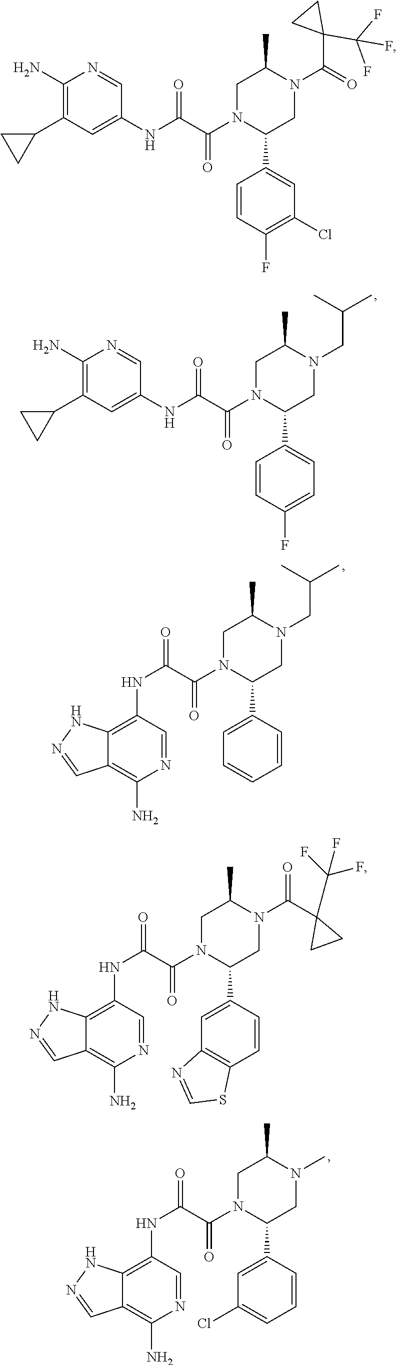 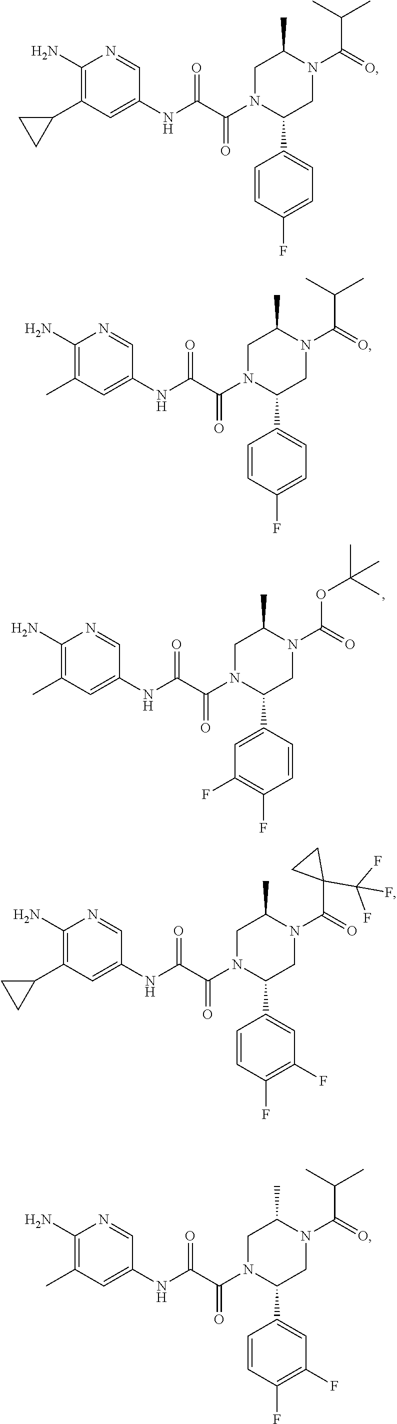 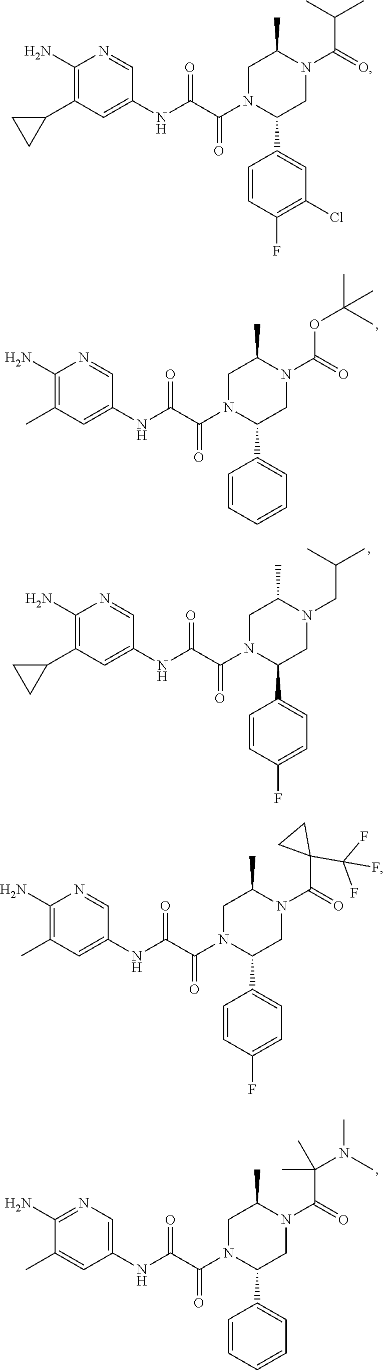 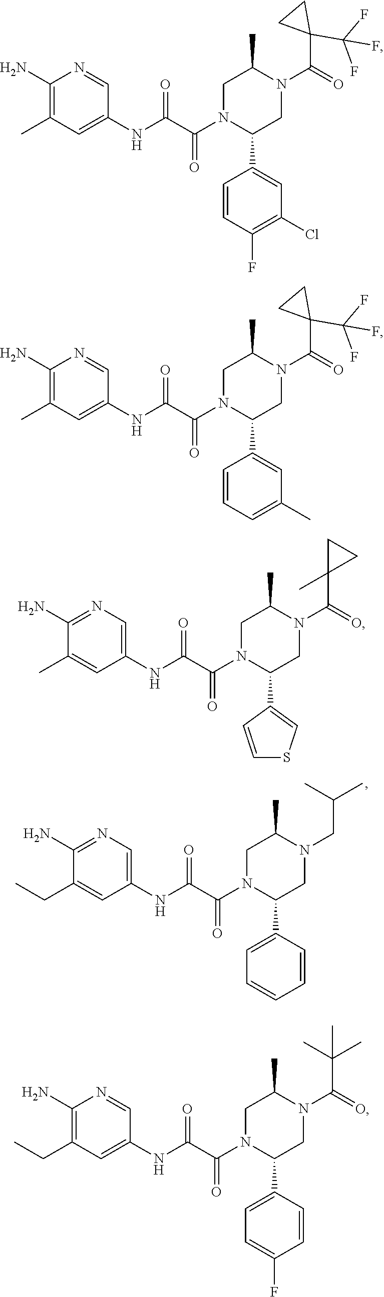 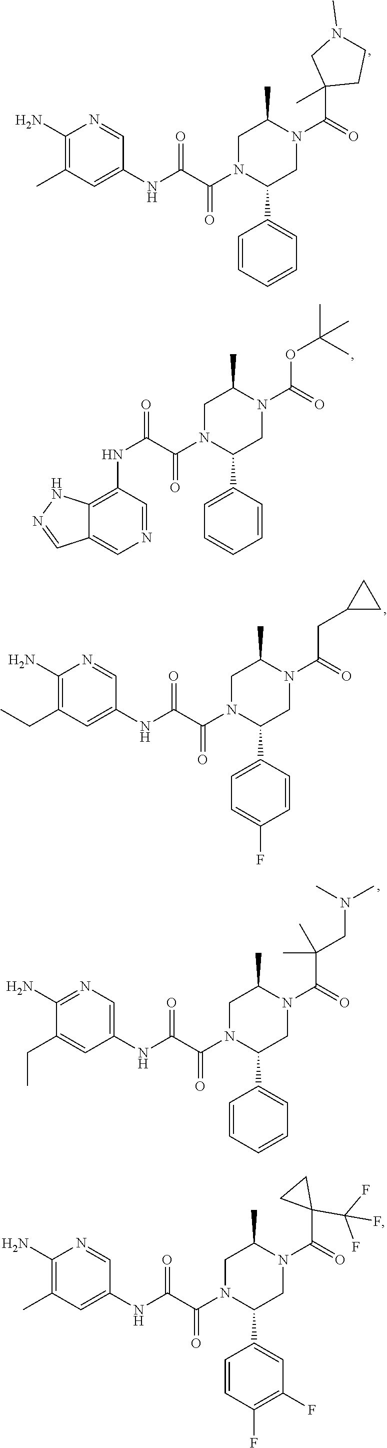 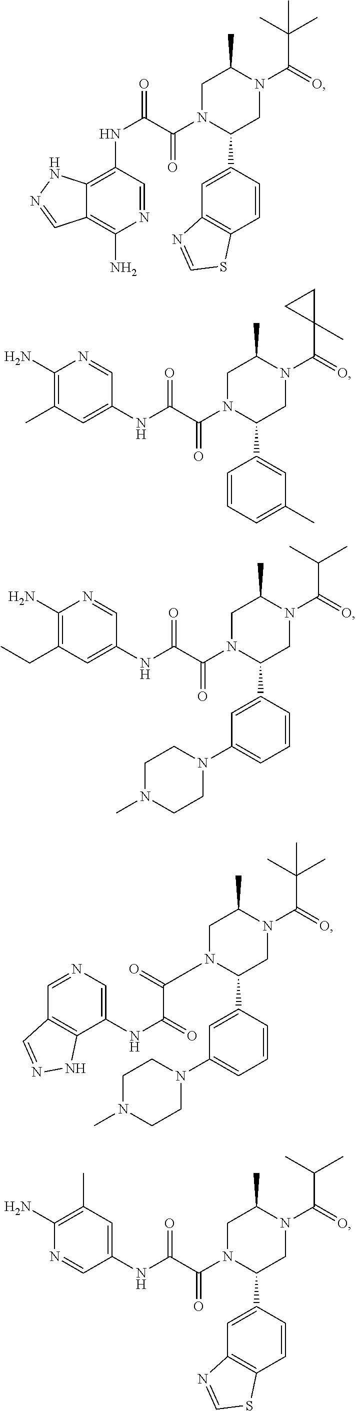 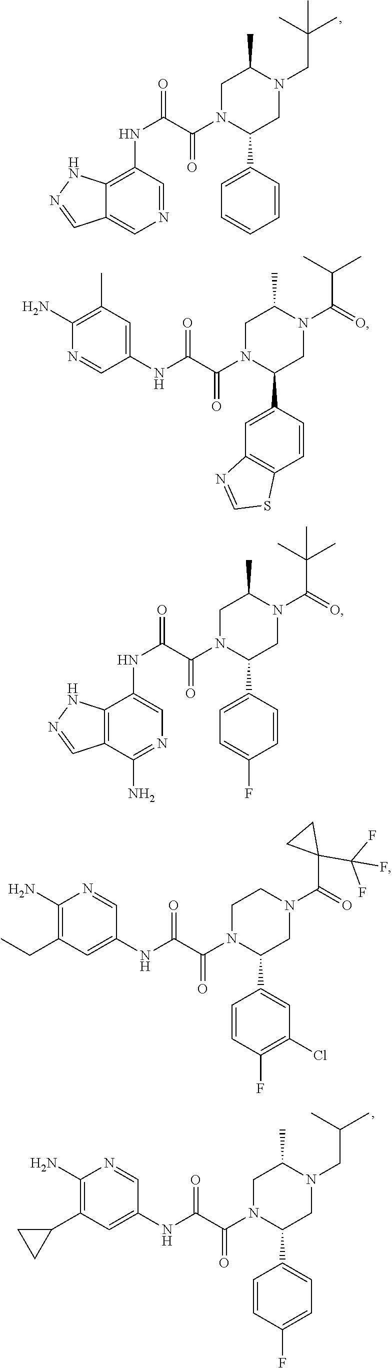 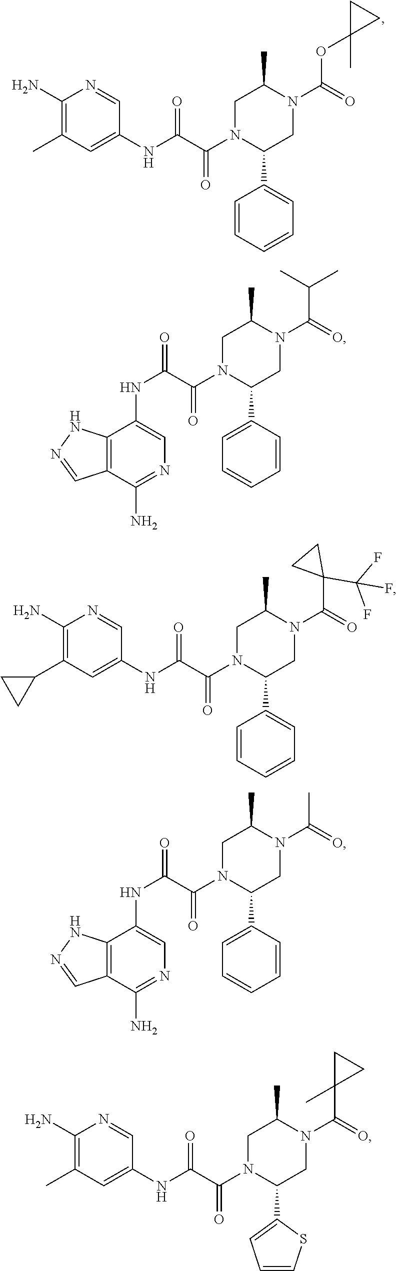 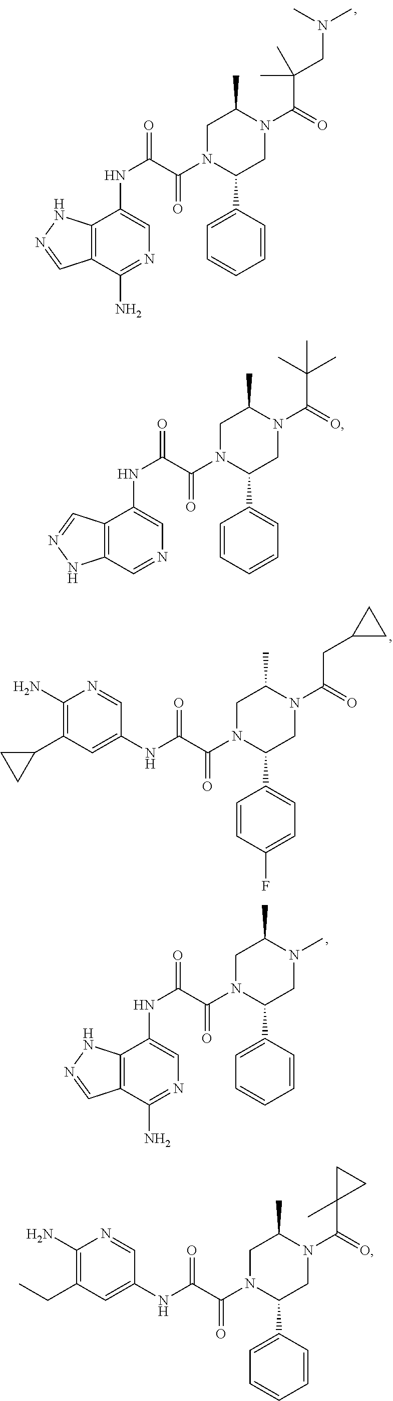 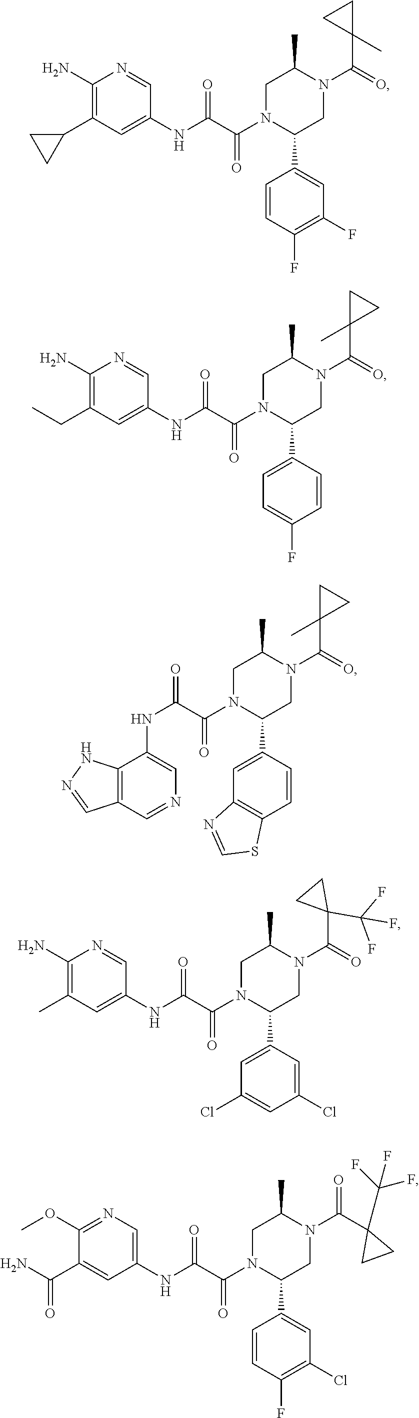 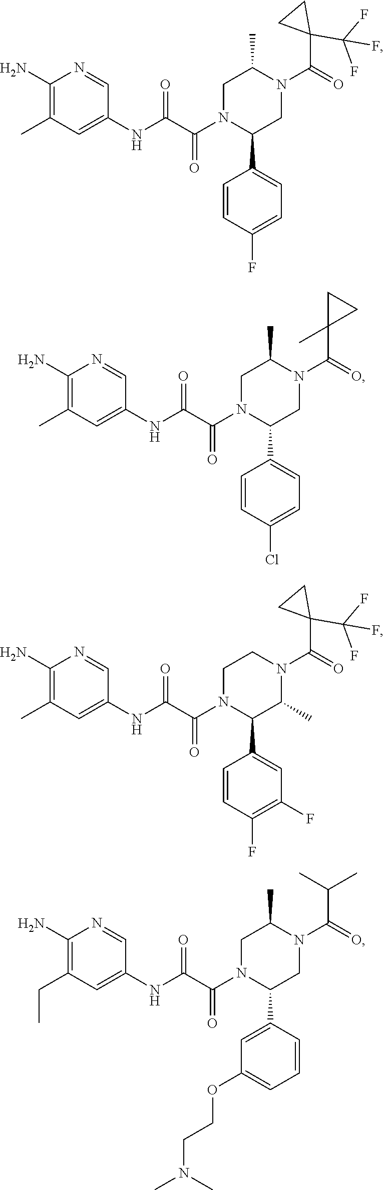 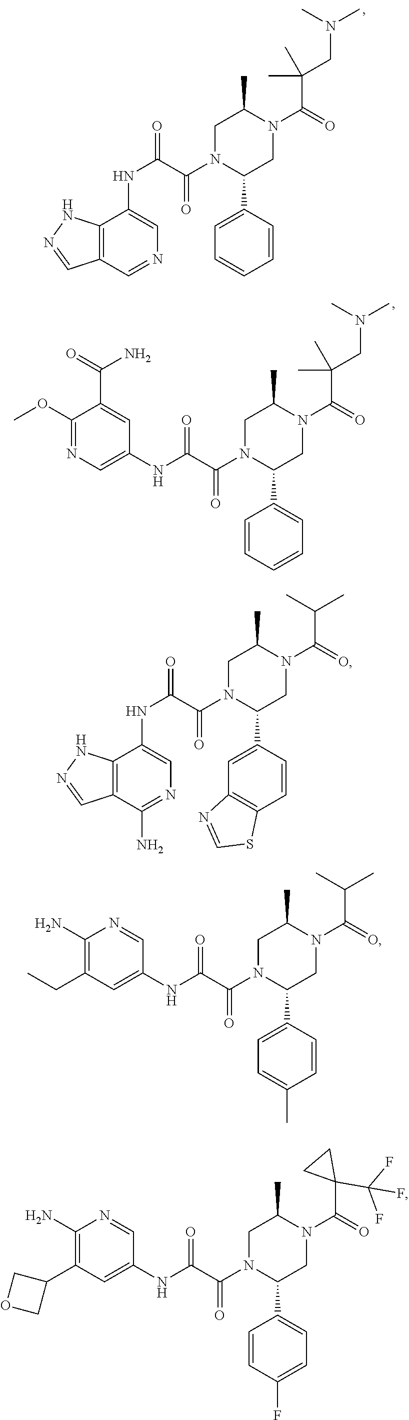 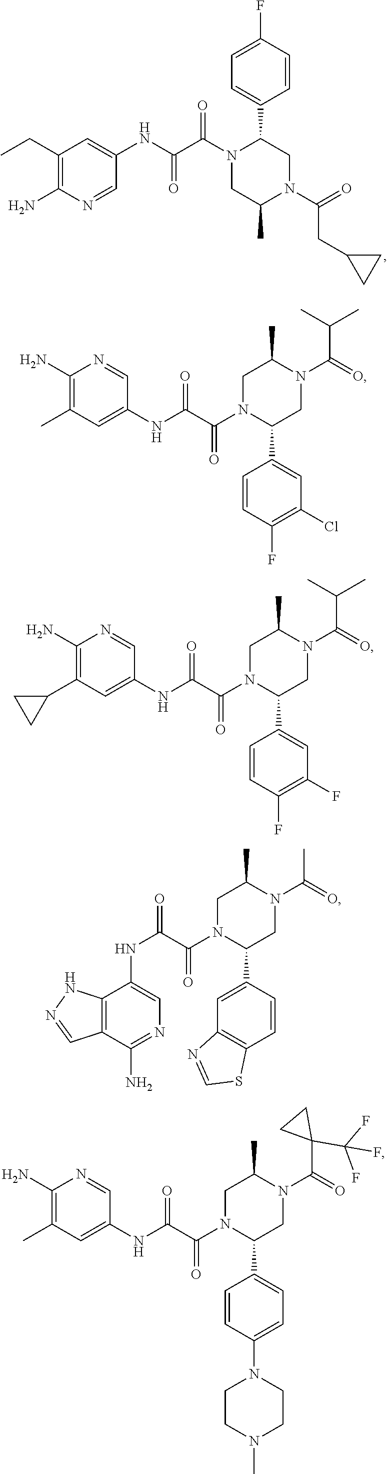 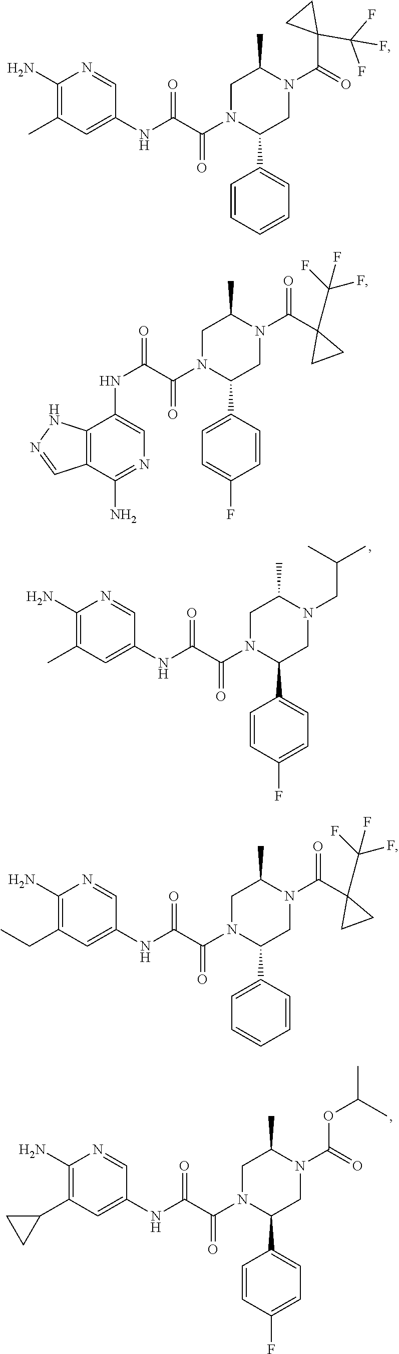 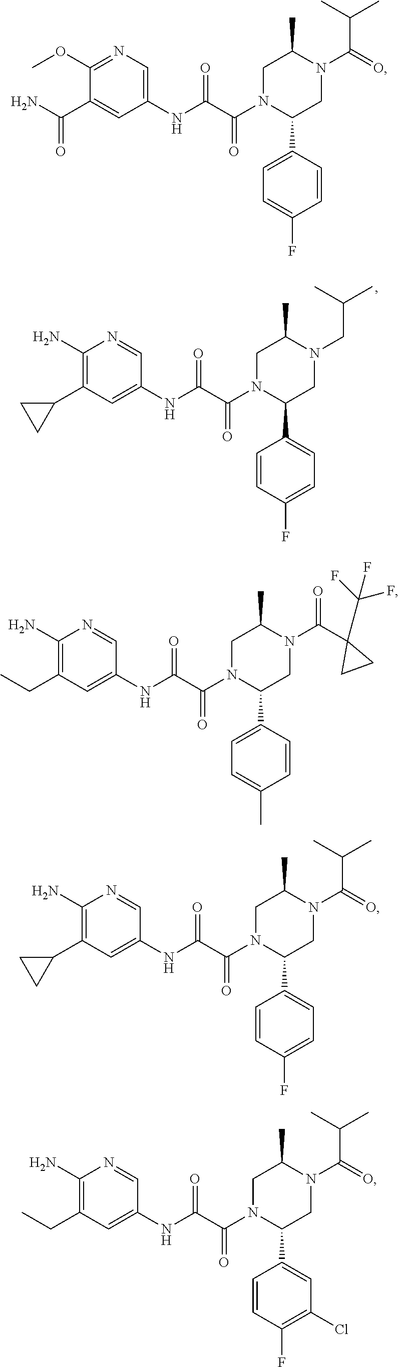 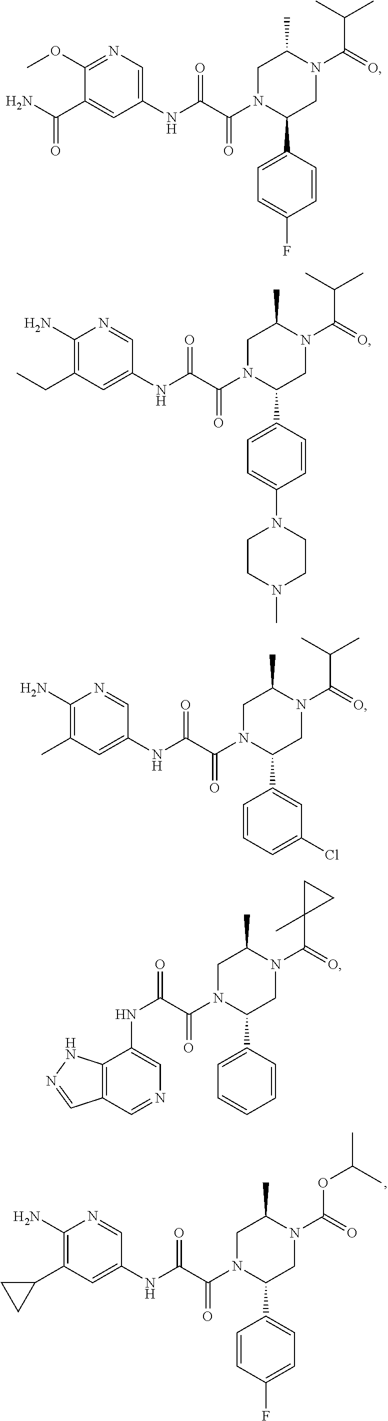 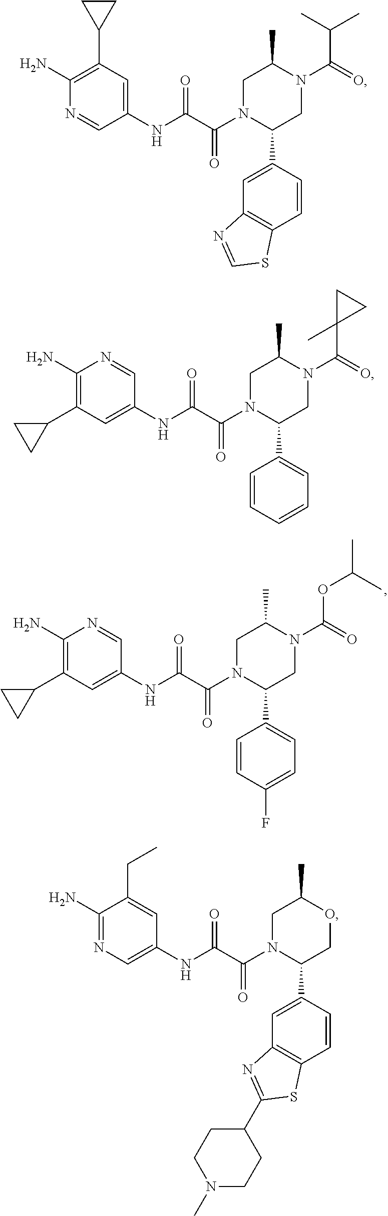 [SMILES: CC(C)C(=O)N1C[C@H](c2ccc3scnc3c2)N(C(=O)C(=O)Nc2cnc(N)c(C3CC3)c2)C[C@H]1C ; C[C@@H]1CN(C(=O)C(=O)Nc2cnc(N)c(C3CC3)c2)[C@@H](c2ccccc2)CN1C(=O)C1(C)CC1 ; CC(C)OC(=O)N1C[C@H](c2ccc(F)cc2)N(C(=O)C(=O)Nc2cnc(N)c(C3CC3)c2)C[C@@H]1C ; CCc1cc(NC(=O)C(=O)N2C[C@@H](C)OC[C@@H]2c2ccc3sc(C4CCN(C)CC4)nc3c2)cnc1N] 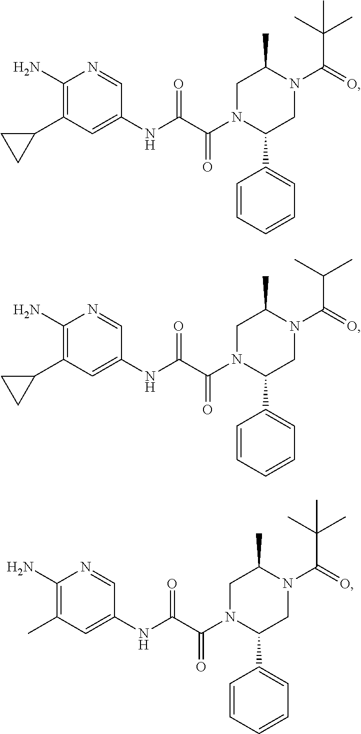 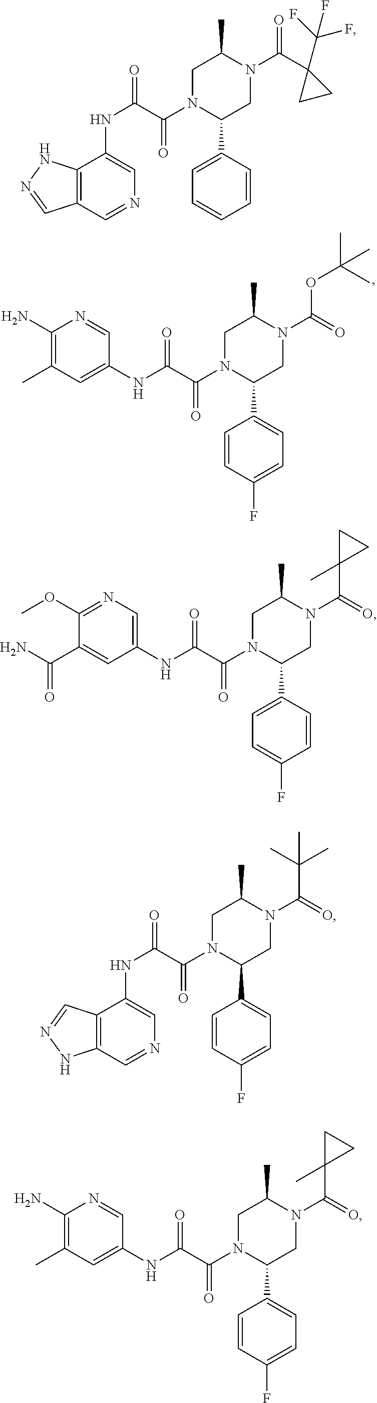 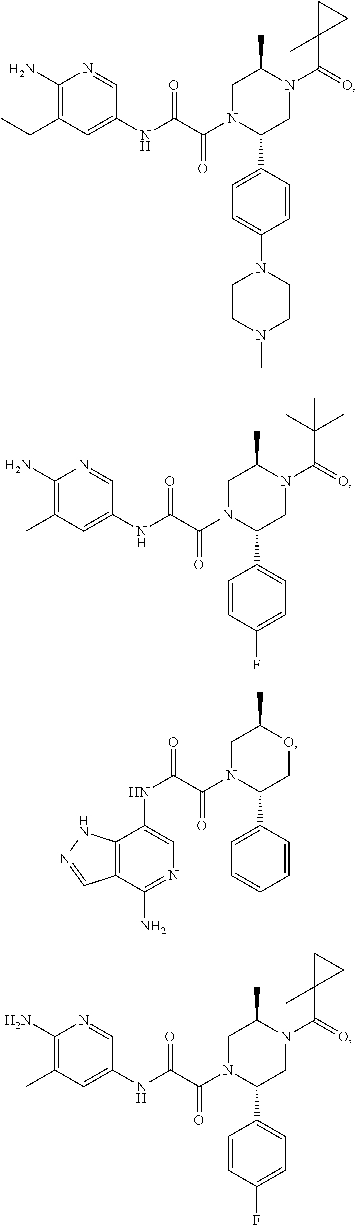 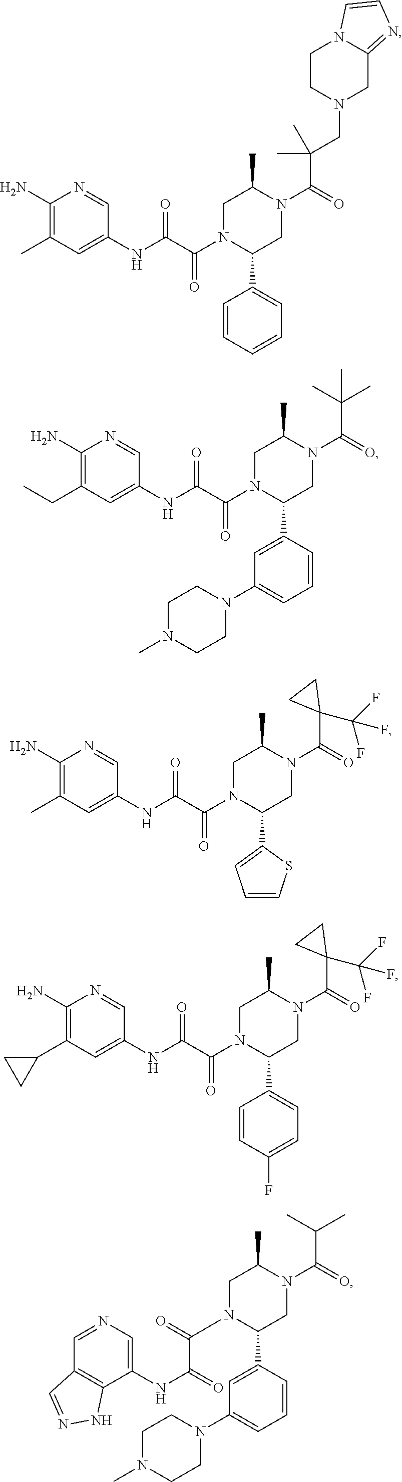 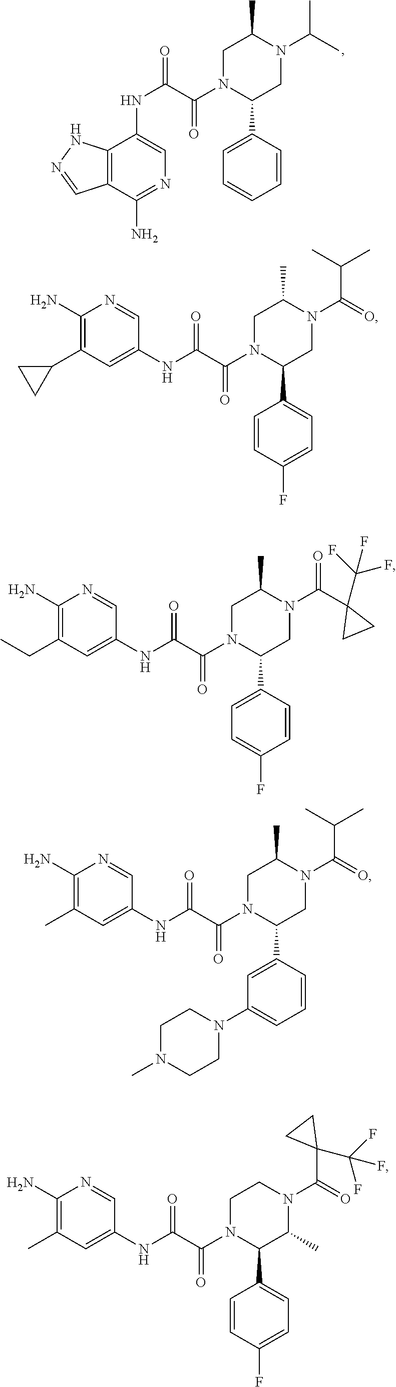 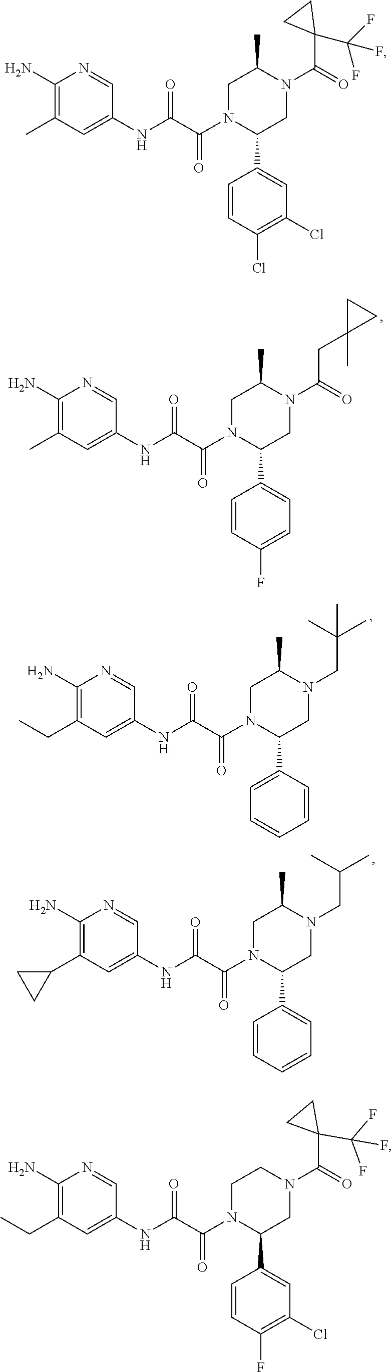 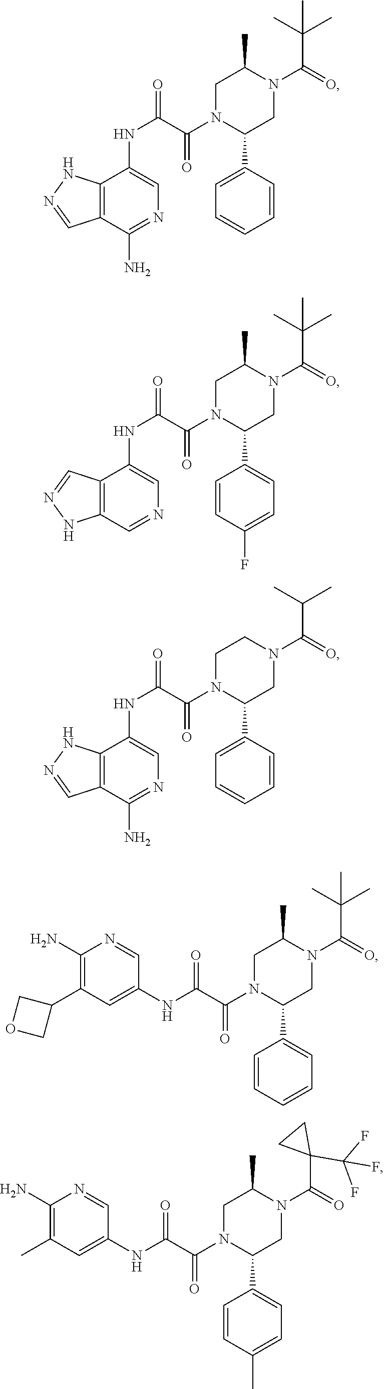 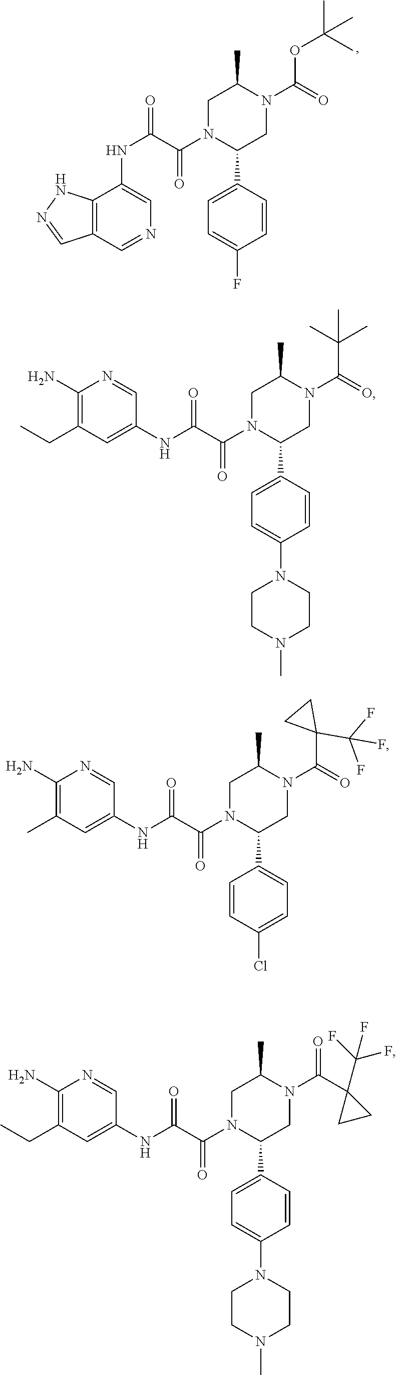 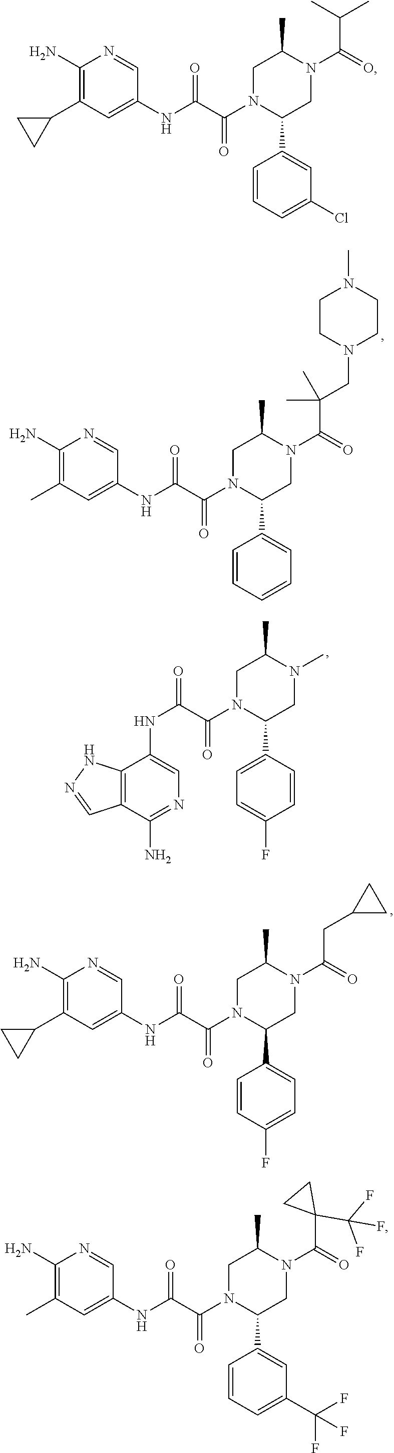 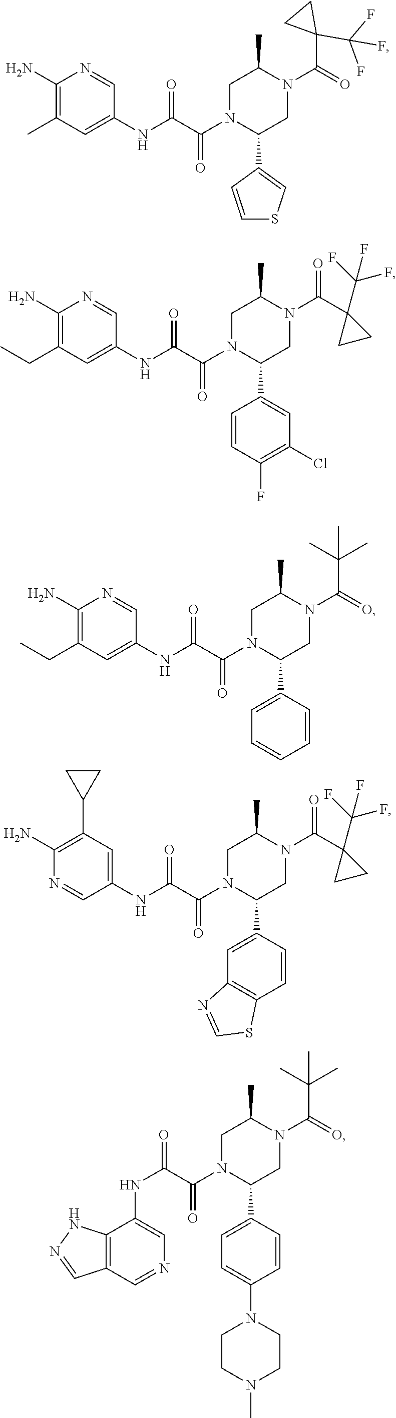 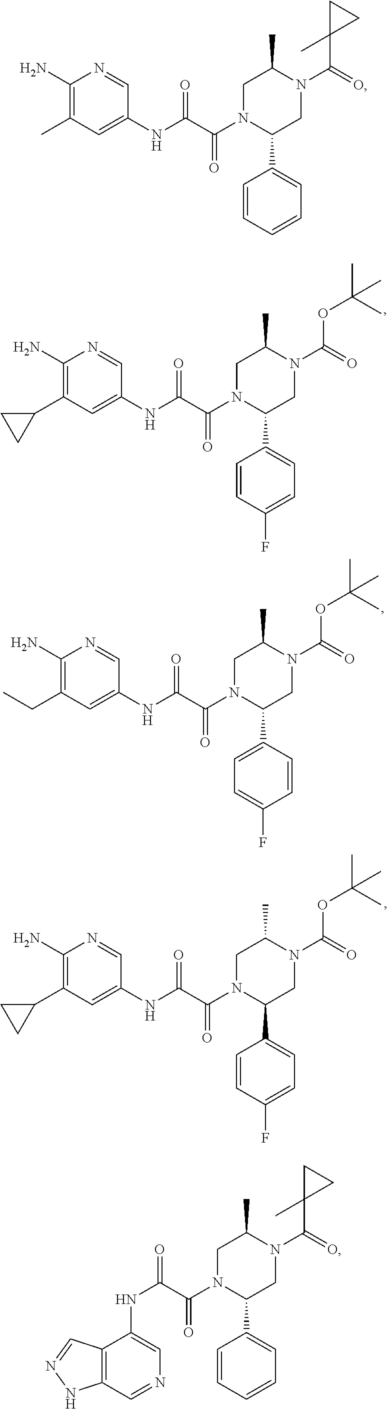 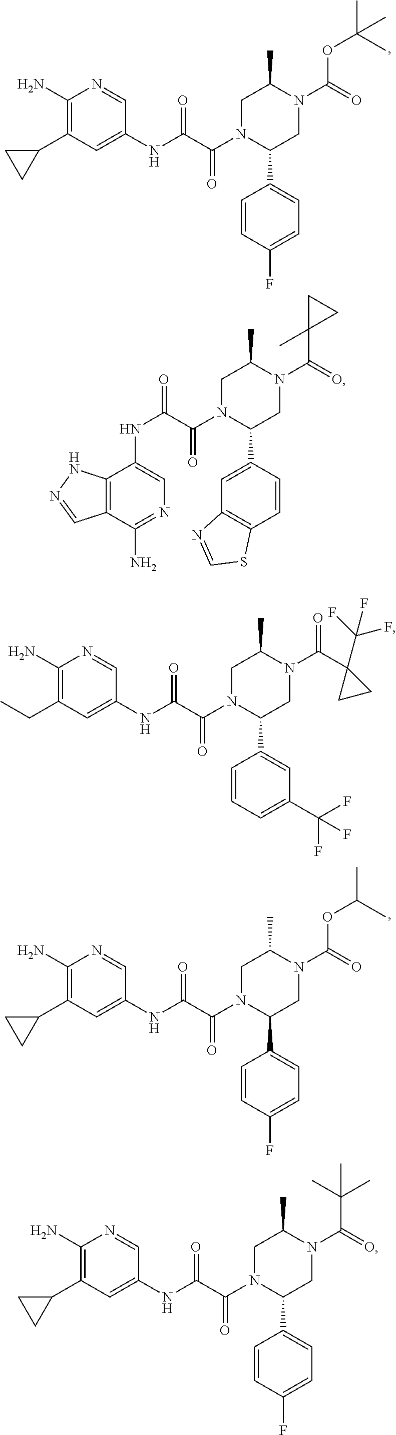 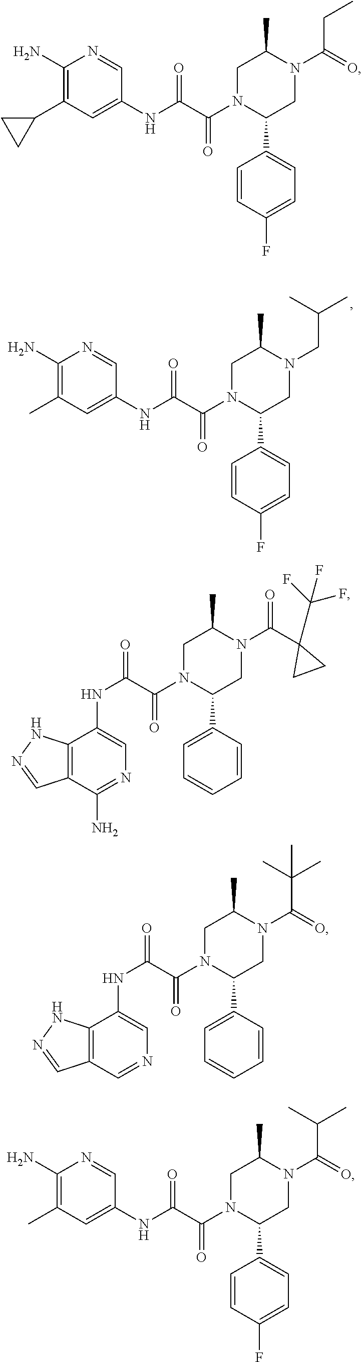 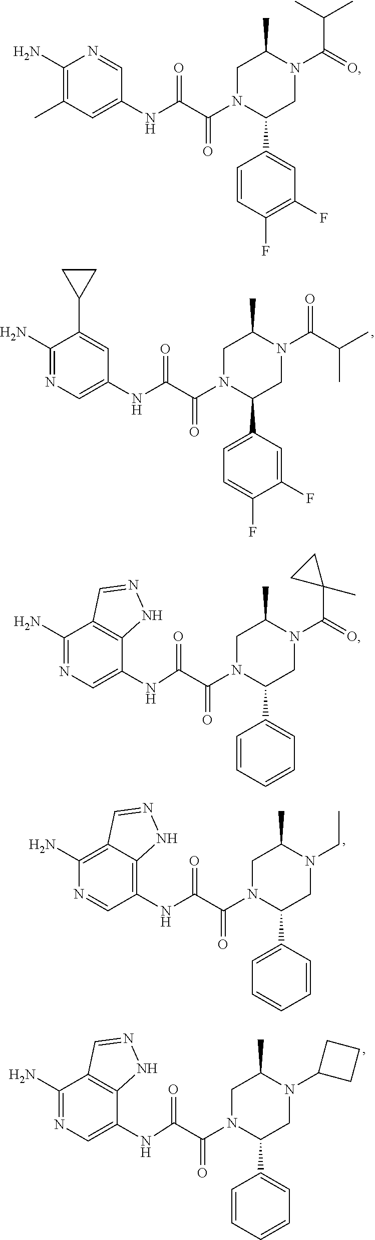 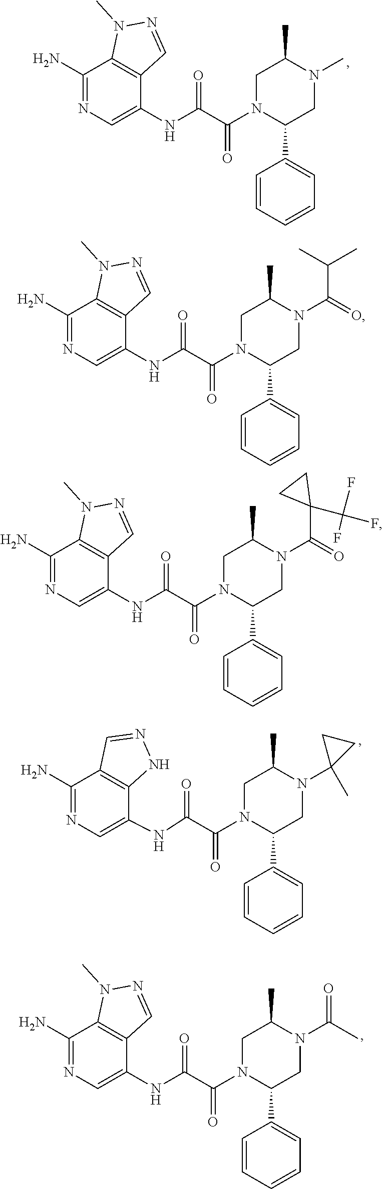 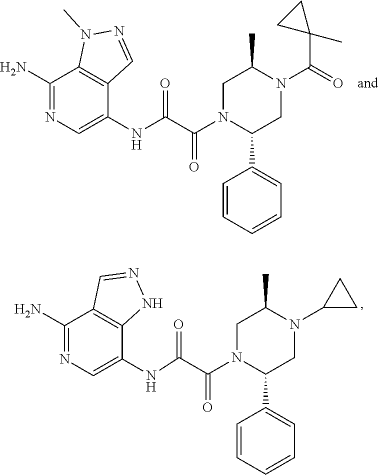
or a pharmaceutically acceptable salt thereof.

87. A pharmaceutical composition comprising a compound of any one of claims 11 to 86, or a pharmaceutically acceptable salt thereof, and a pharmaceutically acceptable carrier.

88. The pharmaceutical composition of claim 87, further comprising a second therapeutic agent.

89. A compound of any one of claims 11 to 86, or a pharmaceutically acceptable salt thereof, or a pharmaceutically acceptable composition of claim 87 for treating an MTAP-deficient and/or an MTA-accumulating disease in a subject in need thereof.

90. The compound or composition for use of claim 89 wherein the compound, or a pharmaceutically acceptable salt thereof, or composition is configured to be administered in combination with a second therapeutic agent.

91. A pharmaceutically acceptable composition of claim 88 for use in treating an MTAP-deficient and/or an MTA-accumulating disease in a subject in need thereof.

92. The compound or composition for use of any one of claims 89 to 91 wherein the disease is a proliferating disease.

93. The compound or composition for use of claim 92 wherein the disease is an MTAP-deficient and/or MTA-accumulating cancer.

94. The compound or composition for use of claim 93 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.

95. Use of a compound of any one of claims 1 to 86, or a pharmaceutically acceptable salt thereof, or of a pharmaceutically acceptable composition of claim 87 in the manufacturing of a medicament for treating an MTAP-deficient and/or an MTA-accumulating disease in a subject in need thereof.

96. The use of claim 95 wherein the medicament is configured to be administered in combination with a second therapeutic agent.

97. Use of a pharmaceutically acceptable composition of claim 88 in the manufacturing of a medicament for treating an MTAP-deficient and/or an MTA-accumulating disease in a subject in need thereof.

98. The use of any one of claims 95 to 97 wherein the disease is a proliferating disease.

99. The use of claim 98 wherein the disease is an MTAP-deficient and/or MTA-accumulating cancer.

100. The use of claim 99 wherein the cancer is glioma, glioblastoma, malignant peripheral nerve sheath tumors (MPNST), esophageal cancer (e.g., esophageal squamous cell carcinoma or esophageal adenocarcinoma), bladder cancer (e.g., bladder urothelial carcinoma), pancreatic cancer (e.g., pancreatic adenocarcinoma), mesothelioma, melanoma, non-small cell lung cancer (NSCLC; e.g., lung squamous or lung adenocarcinoma), astrocytoma, undifferentiated pleiomorphic sarcoma, diffuse large B-cell lymphoma (DLBCL), leukemia, head and neck cancer, stomach adenocarcinoma, myxofibrosarcoma, cholangiosarcoma, cancer of the brain, stomach, kidney, breast, endometrium, urinary tract, liver, soft tissue, pleura and large intestine or sarcoma.
